(19) United States
(12) Patent Application Publication
(10) Pub. No.: US 2005/0154042 A1
(43) Pub. Date: Jul. 14, 2005

(54) N-alkyl pyrroles as HMG-CoA reductase inhibitors

(76) Inventors: Larry D. Bratton, Whitmore Lake, MI (US); Xue-Min Cheng, Ann Arbor, MI (US); Chitase Lee, Ann Arbor, MI (US); Steven R. Miller, Ann Arbor, MI (US); Jeffrey A. Pfefferkorn, Dexter, MI (US); Toni-Jo Poel, Pinckney, MI (US); Roderick J. Sorenson, Ann Arbor, MI (US); Yuntao Song, Ann Arbor, MI (US); Kuai-Lin Sun, Canton, MI (US); Bharat K. Trivedi, Farmington Hills, MI (US); Paul C. Unangst, Ann Arbor, MI (US)

Correspondence Address: WARNER-LAMBERT COMPANY, 2800 PLYMOUTH RD, ANN ARBOR, MI, 48105, US

(21) Appl. No.: 11/002,821

(22) Filed: Dec. 2, 2004

Related U.S. Application Data
(60) Provisional application No. 60/527,347, filed on Dec. 5, 2003.
(60) Provisional application No. 60/601,943, filed on Aug. 16, 2004.
(60) Provisional application No. 60/621,101, filed on Oct. 25, 2004.

(51) Int. Cl. C07D 207/46; A61K 31/40

(52) U.S. Cl. 514/408; 548/571

(57) ABSTRACT

HMGCo-A reductase inhibitor compounds useful as hypocholesterolemic and hypolipidemic compounds are provided. Also provided are pharmaceutical compositions of the compounds. Methods of making and methods of using the compounds are also provided.

### FIELD OF THE INVENTION

The present invention relates to compounds and pharmaceutical compositions useful as hypocholesterolemic and hypolipidemic agents. More specifically, the present invention concerns certain potent inhibitors of the enzyme 3-hydroxy-3-methylglutaryl-coenzyme A reductase (“HMG CoA reductase”). The invention further relates to methods of using such compounds and compositions to treat subjects, including humans, suffering from hyperlipidemia, hypercholesterolemia, hypertriglyceridemia, atherosclerosis, Alzheimer's Disease, BPH, diabetes and osteoporosis.

### BACKGROUND OF THE INVENTION

High levels of blood cholesterol and blood lipids are conditions involved in the onset of atherosclerosis. The conversion of HMG-CoA to mevalonate is an early and rate-limiting step in the cholesterol biosynthetic pathway. This step is catalyzed by the enzyme HMG-CoA reductase. Statins inhibit HMG-CoA reductase from catalyzing this conversion. As such, statins are collectively potent lipid lowering agents. Thus, statins are the drugs of first choice for management of many lipid disorders. Representaative statins include atorvastatin, lovastatin, provastatin and simvastatin.

It is known that inhibitors of HMG-CoA reductase are effective in lowering the blood plasma level of low density lipoprotein cholesterol (LDL-C), in man. (cf. M. S. Brown and J. L. Goldstein, New England Journal of Medicine, 305, No. 9, 515-517 (1981). It has been established that lowering LDL-C levels affords protection from coronary heart disease (cf. Journal of the American Medical Association, 251, No. 3, 351-374 (1984). Further, it is known that certain derivatives of mevalonic acid (3,5-dihydroxy-3-methylpentanoic acid) and the corresponding ring-closed lactone form mevalonolactone, inhibit the biosynthesis of cholesterol (cf. F. M. Singer et al., Proc. Soc. Exper. Biol. Med., 102: 370 (1959) and F. H. Hulcher, Arch. Biochem. Biophys., 146: 422 (1971)). U.S. Pat. Nos. 3,983,140; 4,049,495 and 4,137,322 disclose the fermentative production of a natural product, now called compactin, having an inhibitory effect on cholesterol biosynthesis. Compactin has veen shown to have a complex structure which includes a mevalonolactone moiety (Brown et al., J. Chem. Soc. Perkin I (1976) 1165. U.S. Pat. No. 4,255,444 to Oka et al. discloses several synthetic derivatives of mevalonolactone having antilipidemic activity. U.S. Pat. Nos. 4,198,425 and 4,262,013 to Mitsue et al. disclose aralkyl derivatives of mevalonolactone which are useful in the treatment of hyperlipidemia.

Atorvastatin and pharmaceutically acceptable salts thereof are selective, competitive inhibitors of HMG-CoA reductase. As such, atorvastatin calcium is a potent lipid lowering compound and is thus useful as a hypolipidemic and/or hypocholesterolemic agent, as well as in the treatment of osteoporosis and Alzheimer's disease. A number of patents have issued disclosing atorvastatin. These include: U.S. Pat. Nos. 4,681,893; 5,273,995 and 5,969,156, which are incorporated herein by reference.

All statins interfere, to varying degrees, with the conversion of HMG-CoA to the cholesterol precursor mevalonate by HMG-CoA reductase. These drugs share many features, but also exhibit differences in pharmacologic attributes that may contribute to differences in clinical utility and effectiveness in modifying lipid risk factors for coronary heart disease. (Clin. Cardiol. Bol. 26 (Suppl. III), III-32-III-38 (2003). Some of the desirable pharmocologic features with statin therapy include potent reversible inhibition of HMGCoA reductase, the ability to produce large reductions in LDL-C and non-high-density lipoprotein cholesterol (non-HDL-C), the ability to increase HDL cholesterol (HDL-C), tissue selectivity optimal pharmacokinetics, availability of once a day dosing and a low potential for drug-drug interactions. Also desirable is the ability to lower circulating very-low-density-lipoprotein(VLDL) as well as the ability to lower triglyeride levels.

At the present time, the most potent statins display in vitro IC₅₀ values, using purified human HMG-CoA reductase catalytic domain preparations, of between about 5.4 and about 8.0 nM. Am J. Cardiol 2001;87(suppl): 28B-32B; Atheroscer Suppl. 2002; 2: 33-37. Generally, the most potent LDL-C-lowering statins are also the most potent non-HDL-C-lowering statins. Thus, maximum inhibitory activity is desirable. With respect to HDL-C, the known statins generally produce only modest increases in HDL-C. Therefor, the ability to effect greater increases in HDL-C would be advantageous as well.

With respect to tissue selectivity, differences among statins in relative lipophilicity or hydrophilicity may influence drug kinetics and tissue selectivity. Relatively hydrophilic drugs may exhibit reduced access to nonhepatic cells as a result of low passive diffusion and increased relative hepatic cell uptake through selective organic ion transport. In addition, the relative water solubility of a drug may reduce the need for extensive cytochrome P450 (CYP) enzyme metabolism. Many drugs, including the known statins, are metabolized by the CYP3A4 enzyme system. Arch Intern Med 2000; 160: 2273-2280; J Am Pharm Assoc 2000; 40: 637-644. Thus, relative hydrophilicity is desirable with statin therapy.

Two important pharmacokinetic variables for statins are bioavailability and elimination half-life. It would be advantageous to have a statin with limited systemic availability so as to minimize any potential risk of systemic adverse effects, while at the same time having enough systemic availability so that any pleiotropic effects can be observed in the vasculature with statin treatment. Theses pleiotropic effects include improving or restoring endothelial function, enhancing the stability of atherosclerotic plaques, reduction in blood plasma levels of certain markers of inflammation such as C-reactive protein, decreasing oxidative stress and reducing vascular inflammation. Arterioscler Thromb Vasc Biol 2001; 21: 1712-1719; Heart Dis 5(1): 2-7, 2003. Further, it would be advantageous to have a statin with a long enough elimination half-life to maximize effectiveness for lowering LDL-C.

Finally, it would be advantageous to have a statin that is either not metabolized or minimally metabolized by the CYP3A4 systems so as to minimize any potential risk of drug-drug interactions when statins are given in combination with other drugs.

Accordingly, it would be most beneficial to provide a statin having a combination of desirable properties including high potency in inhibiting HMG-CoA reductase, the ability to produce large reductions in LDL-C and non-high density lipoprotein cholesterol, the ability to increase HDL cholesterol, selectivity of effect or uptake in hepatic cells, optimal systemic bioavailability, prolonged elimination half-life, and absence or minimal metabolism via the CYP3A4 system.

### SUMMARY OF THE INVENTION

This invention provides a novel series of N-alkyl pyrroles as HMG-CoA reductase inhibitors. Compounds of the invention are potent inhibitors of cholesterol biosynthesis. Accordingly, the compounds find utility as therapeutic agents to treat hyperlipidemia, hypercholesterolemia, hypertriglyceridemia and atherosclerosis. More specifically, the present invention provides a compound having a Formula I, 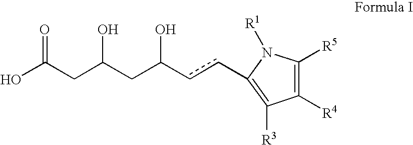
or a pharmaceutically acceptable salt, ester, amide, stereoisomer or prodrug thereof, or a pharmaceutically acceptable salt of the prodrug,
wherein R¹ is lower alkyl, optionally substituted with a halogen;
R³ is benzyl; naphthyl; C₃-C₈ cycloalkyl or C₅-C₈ cycloalkenyl, optionally substituted with one or more heteroatom(s); phenyl or phenyl substituted with one or more groups selected from fluorine, chlorine, bromine, hydroxyl or alkyl of from one to seven carbon atoms; pyridinyl or pyridinyl substituted with fluorine, chlorine, bromine, hydroxyl or alkyl of from one to seven carbon atoms;
R⁴ is H; aryl, aralkyl, heteroaryl or heteroaralkyl; optionally substituted with one or more groups selected from fluorine, chlorine, bromine, hydroxyl or alkyl of from one to seven carbon atoms;
C₁-C₈ alkyl or C₃-C₈ cycloalkyl; optionally substituted; aralkenyl; carbamoyl or substituted carbamoyl; carboxyl or substituted carboxyl;
R⁵ is H, I, phenyl, COOR′, R⁶R⁷NC(O)—, —(CH₂)ₙNR⁶R⁷, or SO₂NR⁶R⁷;
R⁶ and R⁷ are each independently H; aryl, aralkyl, heteroaryl or heteroaralkyl; optionally substituted with halo, alkyl of from one to seven carbon atoms,
(CH₂)ₙOR′, (CH₂)ₙCOOR′, (CH₂)ₙCONR′R″, (CH₂)ₙS(O)₂NR′R″,
(CH₂)ₙS(O)₂R⁸, or heteroaryl;
C₁-C₁₀ alkyl, C₃-C₈ cycloalkyl or C₅-C₈ cycloalkenyl, said alkyl, cycloalkyl or cycloalkenyl optionally containing one or more heteroatoms(s); unsubstituted or substituted with OH, CO₂R′ or CONR′R″; COOR′; C(O)R′; SO₂NHR⁸ or SO₂R⁸;
or N, R⁶ and R⁷ taken together form a 4-7 member ring, optionally containing up to 2 heteroatoms selected from O, N and S, said heteroatom(s) being optionally substituted; said ring optionally substituted with lower alkyl, OH, benzyl, phenyl,
CO₂R′ or CONR′R″;
R⁸ is aryl, aralkyl, alkyl, heteroaryl or heteroaralkyl; optionally substituted;
R′ and R″ are each independently H, C₁-C₁₂ alkyl, aryl, or aralkyl, or taken together form a 4-7 member ring;
n is 0-2; and wherein 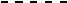
   is a bond or is absent.

The present invention provides inter alia the following compounds: (3R,5R)-7-[3-(4-fluoro-phenyl)-1-isopropyl-4-phenyl-5-phenylcarbamoyl-1H-pyrrol-2-yl)-3,5-dihydroxy-heptanoic acid;
(3R,5R)-7-[3-(4-Fluoro-phenyl)-1-isopropyl-4-phenyl-5-phenylcarbamoyl-1H-pyrrol-2-yl]-3,5-dihydroxy-hept-6-enoic acid;
(3R)-7-[3-(4-Fluoro-phenyl)-1-isopropyl-4-phenyl-5-phenylcarbamoyl-1H-pyrrol-2-yl]-3,5-dihydroxy-heptanoic acid;
(3R,5R)-7-[3-(4-Fluoro-phenyl)-1-isopropyl-4-phenyl-5-(4-sulfamoyl-phenylcarbamoyl)-1H-pyrrol-2-yl]-3,5-dihydroxy-heptanoic acid;
(3R,5S)-7-[3-(4-Fluoro-phenyl)-1-isopropyl-4-phenyl-5-(4-sulfamoyl-phenylcarbamoyl)-1H-pyrrol-2-yl]-3,5-dihydroxy-hept-6-enoic acid;
(3R,5R)-7-[3-(4-Fluoro-phenyl)-5-(4-fluoro-phenylcarbamoyl)-1-isopropyl-4-phenyl-1H-pyrrol-2-yl]-3,5-dihydroxy-heptanoic acid;
(3R,5S)-7-[3-(4-Fluoro-phenyl)-5-(4-fluoro-phenylcarbamoyl)-1-isopropyl-4-phenyl-5-phenylcarbamoyl-1H-pyrrol-2-yl]-3,5-dihydroxy-hept-6-enoic acid;
(3R)-7-[3-(4-Fluoro-phenyl)-5-(4-fluoro-phenylcarbamoyl)-1-isopropyl-4-phenyl-1H-pyrrol-2-yl]-3,5-dihydroxy-heptanoic acid;
(3R,5R)-7-[5-(4-Fluoro-benzylcarbamoyl)-3-(4-fluoro-phenyl)-1-isopropyl-4-phenyl-1H-pyrrol-2-yl]-3,5-dihydroxy-heptanoic acid;
(3R,5S)-7-[5-(4-Fluoro-benzylcarbamoyl)-3-(4-fluoro-phenyl)-1-isopropyl-4-phenyl-1H-pyrrol-2-yl]-3,5-dihydroxy-hept-6-enoic acid;
(3R,5R)-7-[3,4-Bis-(4-fluoro-phenyl)-1-isopropyl-5-(4-sulfamoyl-phenylcarbamoyl)-1H-pyrrol-2-yl]-3,5-dihydroxy-heptanoic acid;
(3R,5S)-7-[3,4-Bis-(4-fluoro-phenyl)-1-isopropyl-5-(4-sulfamoyl-phenycarbamoyl-1H-pyrrol-2-yl]-3,5-dihydroxy-hept-6-enoic acid;
(3R,5R)-7-[5-(4-Carbamoylmethyl-phenylcarbamoyl)-3,4-bis-(4-fluoro-phenyl)-1-isopropyl-)-1H-pyrrol-2-yl]-3,5-dihydroxy-heptanoic acid;
(3R,5S)-7-[5-(4-Carbamoylmethyl-phenylcarbamoyl)-3,4-bis-(4-fluoro-phenyl)-1-isopropyl-1H-pyrrol-2-yl]-3,5-dihydroxy-hept-6-enoic acid;
(3R,5R)-7-[3,4-bis(4-fluorophenyl)-1-isopropyl-5-phenylcarbamoyl-1H-pyrrol-2-yl]-3,5-dihydroxy-heptanoic acid;
(3R,5R)-7-[3,4-bis(4-fluorophenyl)-5-(2-fluoro-phenylcarbamoyl)-1-isopropyl-1H-pyrrol-2-yl]-3,5-dihydroxy-heptanoic acid;
(3R,5S)-7-[3,4-bis(4-fluorophenyl)-5-(4-fluorophenylcarbamoyl)-1-isopropyl-1H-pyrrol-2-yl]-3,5-dihydroxy-hept-6-enoic acid;
(3R,5R)-7-[5-(2,4-difluoro-phenylcarbamoyl)-3,4-bis(4-fluoro-phenyl)-1-isopropyl-1H-pyrrol-2-yl]-3,5-dihydroxy-heptanoic acid;
(3R,5R)-7-[3,4-bis-(4-fluorophenyl)-1-isopropyl-5-p-tolylcarbamoyl-1H-pyrrol-2-yl]-3,5-dihydroxy-heptanoic acid;
(3R,5R)-7-[3,4-bis(4-fluorophenyl)-1-isopropyl-5-m-tolylcarbamoyl-1H-pyrrol-2-yl]-3,5-dihydroxy-heptanoic acid;
(3R,5R)-7-[1-Ethyl-3-(4-fluoro-phenyl)-4-isopropyl-5-phenylcarbamoyl-1H-pyrrol-2-yl]-3,5-dihydroxy-heptanoic acid;
(3R,5R)-7-[1-ethyl-3-(4-fluoro-phenyl)-4-methyl-5-phenylcarbamoyl-1-Hpyrrol-2-yl]-3,5-dihydroxy-heptanoic acid;
(3R,5R)-7-[3,4-bis(4-fluorophenyl)-1-isopropyl-5-(piperidine-1-carbonyl)-1H-pyrrol-2-yl]-3,5-dihydroxy-heptanoic acid;
(3R,5R)-7-[3;4-Bis-(4-fluoro-phenyl)-1-isopropyl-5-(4-methanesulfonyl-benzylcarbamoyl)-1H-pyrrol-2-yl]-3,5-dihydroxy-heptanoic acid;
(3R,5R)-7-[3,4-Bis-(4-fluoro-phenyl)-1-isopropyl-5-(4-sulfamoyl-benzylcarbamoyl)-1H-pyrrol-2-yl]-3,5-dihydroxy-heptanoic acid;
trans-(3R,5S)-7-[3,4-Bis-(4-fluoro-phenyl)-1-isopropyl-5-(4-sulfamoyl-benzylcarbamoyl)-1H-pyrrol-2-yl]-3,5-dihydroxy-hept-6-enoic acid;
(3R,5R)-7-[5-(4-Dimethylcarbamoyl-benzylcarbamoyl)-3,4-bis-(4-fluoro-phenyl)-1-isopropyl-1H-pyrrol-2-yl]-3,5-dihydroxy-heptanoic acid;
(3R,5R)-7-[3,4-Bis-(4-fluoro-phenyl)-1-isopropyl-5-(4-sulfamoylmethyl-phenylcarbamoyl)-1H-pyrrol-2-yl]-3,5-dihydroxy-heptanoic acid;
trans-(3R,5S)-7-[3,4-Bis-(4-fluoro-phenyl)-1-isopropyl-5-(4-sulfamoylmethyl-phenylcarbamoyl)-1H-pyrrol-2-yl]-3,5-dihydroxy-hept-6-enoic acid;
(3R,5R)-7-[3,4-Bis-(4-fluoro-phenyl)-1-isopropyl-5-(4-methanesulfonylmethyl-phenylcarbamoyl)-1H-pyrrol-2-yl]-3,5-dihydroxy-heptanoic acid;
trans-(3R,5S)-7-[3,4-Bis-(4-fluoro-phenyl)-1-isopropyl-5-(4-methanesulfonylmethyl-phenylcarbamoyl)-1H-pyrrol-2-yl]-3,5-dihydroxy-hept-6-enoic acid;
(3R,5R)-7-{3-(4-Fluoro-phenyl)-1-isopropyl-4-phenyl-5-[(pyridin-2-ylmethyl)-carbamoyl]-1H-pyrrol-2-yl}-3,5-dihydroxy-heptanoic acid;
trans-(3R,5S)-7-{3-(4-Fluoro-phenyl)-1-isopropyl-4-phenyl-5-[(pyridin-2-ylmethyl)-carbamoyl]-1H-pyrrol-2-yl}-3,5S-dihydroxy-hept-6-enoic acid;
(3R,5R)-7-[5-(3-Dimethylcarbamoyl-phenylcarbamoyl)-3-(4-fluoro-phenyl)-1-isopropyl-4-phenyl-1H-pyrrol-2-yl]-3,5-dihydroxy-heptanoic acid;
trans-(3R,5S)-7-[5-(3-Dimethylcarbamoyl-phenylcarbamoyl)-3-(4-fluoro-phenyl)-1-isopropyl-4-phenyl-1H-pyrrol-2-yl]-3,5-dihydroxy-hept-6-enoic acid;
(3R,5R)-3-{[5-(6-Carboxy-3,5-dihydroxy-hexyl)-4-(4-fluoro-phenyl)-1-isopropyl-3-phenyl-1H-pyrrole-2-carbonyl]-amino}-benzoic acid methyl ester;
(3R,5R)-3-{[5-(6-Carboxy-3,5-dihydroxy-hexyl)-4-(4-fluoro-phenyl)-1-isopropyl-3-phenyl-1H-pyrrole-2-carbonyl]-amino}-benzoic acid;
trans-(3R,5S)-3-{[5-(6-Carboxy-3,5-dihydroxy-hex-1-enyl)-4-(4-fluoro-phenyl)-1-isopropyl-3-phenyl-1H-pyrrole-2-carbonyl]-amino}-benzoic acid methyl ester;
trans-(3R,5S)-3-{[5-(6-Carboxy-3,5-dihydroxy-hex-1-enyl)-4-(4-fluoro-phenyl)-1-isopropyl-3-phenyl-1H-pyrrole-2-carbonyl]-amino}-benzoic acid;
(3R,5R)-7-[3-(4-Fluoro-phenyl)-1-isopropyl-5-(5-methyl-isoxazol-3-ylcarbamoyl)-4-phenyl-1H-pyrrol-2-yl]-3,5-dihydroxy-heptanoic acid;
trans-(3R,5S)-7-[3-(4-Fluoro-phenyl)-1-isopropyl-5-(5-methyl-isoxazol-3-ylcarbamoyl)-4-phenyl-H-pyrrol-2-yl]-3,5-dihydroxy-hept-6-enoic acid;
(3R,5R)-7-[3-(4-Fluoro-phenyl)-1-isopropyl-5-(4-methyl-pyrimidin-2-ylcarbamoyl)-4-phenyl-1H-pyrrol-2-yl]-3,5-dihydroxy-heptanoic acid;
trans-(3R,5S)-7-[3-(4-Fluoro-phenyl)-1-isopropyl-5-(4-methyl-pyrimidin-2-ylcarbamoyl)-4-phenyl-1H-pyrrol-2-yl]-3,5-dihydroxy-hept-6-enoic acid;
(3R,5R)-7-[3-(4-Fluoro-phenyl)-1-isopropyl-5-(3-oxazol-2-yl-phenylcarbamoyl)-4-phenyl-1H-pyrrol-2-yl]-3,5-dihydroxy-heptanoic acid;
trans-(3R,5S)-7-[3-(4-Fluoro-phenyl)-1-isopropyl-5-(3-oxazol-2-yl-phenylcarbamoyl)-4-phenyl-H-pyrrol-2-yl]-3,5-dihydroxy-hept-6-enoic acid;
(3R,5R)-7-[3-(4-Fluoro-phenyl)-1-isopropyl-5-(4-oxazol-2-yl-phenylcarbamoyl)-4-phenyl-1H-pyrrol-2-yl]-3,5-dihydroxy-heptanoic acid;
trans-(3R,5S)-7-[3-(4-Fluoro-phenyl)-1-isopropyl-5-(4-oxazol-2-yl-phenylcarbamoyl)-4-phenyl-1H-pyrrol-2-yl]-3,5-dihydroxy-hept-6-enoic acid;
(3R,5R)-7-[3-(4-Fluoro-phenyl)-1-isopropyl-4-phenyl-5-(pyrimidin-2-ylcarbamoyl)-1H-pyrrol-2-yl]-3,5-dihydroxy-heptanoic acid;
trans-(3R,5S)-7-[3-(4-Fluoro-phenyl)-1-isopropyl-4-phenyl-5-(pyrimidin-2-ylcarbamoyl)-1H-pyrrol-2-yl]-3,5-dihydroxy-hept-6-enoic acid;
trans-(3R,5S)-7-[5-(4-Dimethylcarbamoyl-phenylcarbamoyl)-3,4-bis-(4-fluoro-phenyl)-1-isopropyl-1H-pyrrol-2-yl]-3,5-dihydroxy-hept-6-enoic acid;
(3R,5R)-7-[5-(4-Dimethylcarbamoyl-phenylcarbamoyl)-3,4-bis-(4-fluoro-phenyl)-1-isopropyl-1H-pyrrol-2-yl]-3,5-dihydroxy-heptanoic acid;
(3R)-7-[5-(4-Dimethylcarbamoyl-phenylcarbamoyl)-3,4-bis-(4-fluoro-phenyl)-1-isopropyl-1H-pyrrol-2-yl]-3-hydroxy-heptanoic acid;
trans-(3R,5S)-7-[5-(4-Dimethylsulfamoyl-benzylcarbamoyl)-3,4-bis-(4-fluoro-phenyl)-1-isopropyl-1H-pyrrol-2-yl]-3,5-dihydroxy-hept-6-enoic acid;
(3R,5R)-7-[5-(4-Dimethylsulfamoyl-benzylcarbamoyl)-3,4-bis-(4-fluoro-phenyl)-1-isopropyl-1H-pyrrol-2-yl]-3,5-dihydroxy-heptanoic acid;
trans-(3R,5S)-7-{3,4-Bis-(4-fluoro-phenyl)-1-isopropyl-5-[(pyridin-2-ylmethyl)-carbamoyl]-1H-pyrrol-2-yl}-3,5-dihydroxy-hept-6-enoic acid;
(3R,5R)-7-{3,4-Bis-(4-fluoro-phenyl)-1-isopropyl-5-[(pyridin-2-ylmethyl)-carbamoyl]-1H-pyrrol-2-yl}-3,5-dihydroxy-heptanoic acid;
trans-(3R,5S)-7-[5-(3-Diemethylsulfamoyl-benzylcarbamoyl)-3,4-bis-(4-fluoro-phenyl)-1-isopropyl-1H-pyrrol-2-yl]-3,5-dihydroxy-hept-6-enoic acid;
(3R,5R)-7-[5-(3-Diemethylsulfamoyl-benzylcarbamoyl)-3,4-bis-(4-fluoro-phenyl)-1-isopropyl-1H-pyrrol-2-yl]-3,5-dihydroxy-heptanoic acid;
trans-(3R,5S)-7-[5-(3-Dimethylcarbamoyl-phenylcarbamoyl)-3,4-bis-(4-fluoro-phenyl)-1-isopropyl-1H-pyrrol-2-yl]-3,5-dihydroxy-hept-6-enoic acid;
(3R,5R)-7-[5-(3-Dimethylcarbamoyl-phenylcarbamoyl)-3,4-bis-(4-fluoro-phenyl)-1-isopropyl-1H-pyrrol-2-yl]-3,5-dihydroxy-heptanoic acid;
trans-(3R,5S)-{[5-(6-Carboxy-3,5-dihydroxy-hex-1-enyl)-3,4-bis-(4-fluoro-phenyl)-1-isopropyl-1H-pyrrole-2-carbonyl]-amino}-benzoic acid methyl ester;
(3R,5R)-{[5-(6-Carboxy-3,5-dihydroxy-hexyl)-3,4-bis-(4-fluoro-phenyl)-1-isopropyl-1H-pyrrole-2-carbonyl]-amino}-benzoic acid methyl ester;
trans-(3R,5S)-7-[3,4-Bis-(4-fluoro-phenyl)-1-isopropyl-5-(4-methyl-pyrimidin-2-ylcarbamoyl)-1H-pyrrol-2-yl]-3,5-dihydroxy-hept-6-enoic acid;
(3R,5R)-7-[3,4-Bis-(4-fluoro-phenyl)-1-isopropyl-5-(4-methyl-pyrimidin-2-ylcarbamoyl)-1H-pyrrol-2-yl]-3,5-dihydroxy-heptanoic acid;
trans-(3R,5S)-7-[3,4-Bis-(4-fluoro-phenyl)-1-isopropyl-5-(4-oxazol-2-yl-phenylcarbamoyl)-1H-pyrrol-2-yl]-3,5-dihydroxy-hept-6-enoic acid;
(3R,5R)-7-[3,4-Bis-(4-fluoro-phenyl)-1-isopropyl-5-(4-oxazol-2-yl-phenylcarbamoyl)-1H-pyrrol-2-yl]-3,5-dihydroxy-heptanoic acid;
trans-(3R,5S)-7-[3,4-Bis-(4-fluoro-phenyl)-1-isopropyl-5-(3-oxazol-2-yl-phenylcarbamoyl)-1H-pyrrol-2-yl]-3,5-dihydroxy-hept-6-enoic acid;
(3R,5R)-7-[3.,4-Bis-(4-fluoro-phenyl)-1-isopropyl-5-(3-oxazol-2-yl-phenylcarbamoyl)-1H-pyrrol-2-yl]-3,5-dihydroxy-heptanoic acid;
trans-(3R,5S)-7-[3,4-Bis-(4-fluoro-phenyl)-1-isopropyl-5-(5-methyl-isoxazol-3-ylcarbamoyl)-1H-pyrrole-2-yl]-3,5-dihydroxy-hept-6-enoic acid;
(3R,5R)-7-[3,4-Bis-(4-fluoro-phenyl), 1-isopropyl-5-(5-methyl-isoxazol-3-ylcarbamoyl)-1H-pyrrole-2-yl]-3,5-dihydroxy-heptanoic acid;
(3R,5S)-7-[5-(4-Benzyloxy-phenylcarbamoyl)-3,4-bis-(4-fluoro-phenyl)-1-isopropyl-1H-pyrrol-2-yl]-3,5-dihydroxy-hept-6-enoic acid;
(3R,5S)-7-[3,4-Bis-(4-fluoro-phenyl)-1-isopropyl-5-(4-methoxycarbonyl-benzylcarbamoyl)-1H-pyrrol-2-yl]-3,5-dihydroxy-hept-6-enoic acid;
(3R,5R)-7-[3,4-Bis-(4-fluoro-phenyl)-5-(4-hydroxy-phenylcarbamoyl)-1-isopropyl-1H-pyrrol-2-yl]-3,5-dihydroxy-heptanoic acid;
(3R,5R)-7-[5-(4-Benzyloxy-phenylcarbamoyl)-3,4-bis-(4-fluoro-phenyl)-1-isopropyl-1H-pyrrol-2-yl]-3,5-dihydroxy-heptanoic acid;
(3R,5R)-7-[3,4-Bis-(4-fluoro-phenyl)-1-isopropyl-5-(4-methoxycarbonyl-benzylcarbamoyl)-1H-pyrrol-2-yl]-3,5-dihydroxy-heptanoic acid;
(3R,5S)-7-[5-(2-Benzyloxy-phenylcarbamoyl)-3,4-bis-(4-fluoro-phenyl)-1-isopropyl-1H-pyrrol-2-yl]-3,5-dihydroxy-hept-6-enoic acid;
(3R,5R)-7-[5-(4-Carboxy-benzylcarbamoyl)-3,4-bis-(4-fluoro-phenyl)-1-isopropyl-1H-pyrrol-2-yl]-3,5-dihydroxy-heptanoic acid;
(3R,5S)-7-[5-(3-Benzyloxy-phenylcarbamoyl)-3,4-bis-(4-fluoro-phenyl)-1-isopropyl-1H-pyrrol-2-yl]-3,5-dihydroxy-hept-6-enoic acid;
(3R,5S)-7-[3,4-Bis-(4-fluoro-phenyl)-1-isopropyl-5-(3-sulfamoyl-phenylcarbamoyl)-1H-pyrrol-2-yl]-3,5-dihydroxy-hept-6-enoic acid;
(3R,5R)-7-[3,4-Bis-(4-fluoro-phenyl)-5-(3-hydroxy-phenylcarbamoyl)-1-isopropyl-1H-pyrrol-2-yl]-3,5-dihydroxy-heptanoic acid;
(3R,5R)-7-[3,4-Bis-(4-fluoro-phenyl)-5-(2-hydroxy-phenylcarbamoyl)-1-isopropyl-1H-pyrrol-2-yl]-3,5-dihydroxy-heptanoic acid;
(3R,5S)-7-[3,4-Bis-(4-fluoro-phenyl)-1-isopropyl-5-(3-methoxy-phenylcarbamoyl)-1H-pyrrol-2-yl]-3,5-dihydroxy-hept-6-enoic acid;
(3R,5R)-7-[3,4-Bis-(4-fluoro-phenyl)-1-isopropyl-5-(3-sulfamoyl-phenylcarbamoyl)-1H-pyrrol-2-yl]-3,5-dihydroxy-heptanoic acid;
(3R,5R)-7-[3,4-Bis-(4-fluoro-phenyl)-1-isopropyl-5-(3-methoxy-phenylcarbamoyl)-1H-pyrrol-2-yl]-3,5-dihydroxy-heptanoic acid;
(3R,5S)-7-[5-(3-Chloro-phenylcarbamoyl)-3,4-bis-(4-fluoro-phenyl)-1-isopropyl-1H-pyrrol-2-yl]-3,5-dihydroxy-hept-6-enoic acid;
(3R,5S)-7-[5-(3-Ethyl-phenylcarbamoyl)-3,4-bis-(4-fluoro-phenyl)-1-isopropyl-1H-pyrrol-2-yl]-3,5-dihydroxy-hept-6-enoic acid;
(3R,5R)-7-[5-(3-Ethyl-phenylcarbamoyl)-3,4-bis-(4-fluoro-phenyl)-1-isopropyl-1H-pyrrol-2-yl]-3,5-dihydroxy-heptanoic acid;
(3R,5R)-7-[5-(3-Chloro-phenylcarbamoyl)-3,4-bis-(4-fluoro-phenyl)-1-isopropyl-1H-pyrrol-2-yl]-3,5-dihydroxy-heptanoic acid;
(3R,5R)-7-[3,4-bis(4-fluorophenyl)-5-(4-fluoro-phenylcarbamoyl)-1-isopropyl-1H-pyrrol-2-yl]-3,5-dihydroxy-heptanoic acid;
(3R,5R)-7-[3,4-bis(4-fluoro-phenyl)-5-(3-fluorophenylcarbamoyl)-1-isopropyl-1H-pyrrol-2-yl]-3,5-dihydroxy-heptanoic acid;
(3R,5R)-7-[5-(4-Carboxymethyl-phenylcarbamoyl)-3,4-bis(4-fluorophenyl)-1-isopropyl-1H-pyrrol-2-yl]-3,5-dihydroxy-heptanoic acid;
(3R,5R)-7-[5-(4-ethylpiperazine-1-carbonyl)-3,4-bis(4-fluorophenyl)-1-isopropyl-1H-pyrrol-2-yl]-3,5-dihydroxy-heptanoic acid;
(3R,5R)-7-[5-(4-carbamoyl-phenylcarbamoyl)-3,4-bis(4-fluorophenyl)-1-isopropyl-1H-pyrrol-2-yl]-3,5-dihydroxy-heptanoic acid;
(3R,5R)-7-[3,4-bis(4-fluorophenyl)-1-isopropyl-5-(4-methoxycarbonyl-phenylcarbamoyl)-1H-pyrrol-2-yl]-3,5-dihydroxy-heptanoic acid;
(3R,5R)-7-[3,4-bis-(4-fluorophenyl)-1-isopropyl-5-(4-sulfamoyl-phenylcarbamoyl)-1H-pyrrol-2-yl]-3,5-dihydroxy-heptanoic acid;
(3R,5R)-7-[5-(3,5-difluorophenylcarbamoyl)-3-(4-fluorophenyl)-1-isopropyl-4-phenyl-1H-pyrrol-2-yl]-3,5-dihydroxy-heptanoic acid;
(3R,5R)-7-[3-(4-fluoro-phenyl)-1-isopropyl-4-phenyl-5-(pyridin-2-ylcarbamoyl)-1H-pyrrol-2-yl]-3,5-dihydroxy-heptanoic acid;
   and pharmaceutically acceptable salts, esters and amides thereof.

Further provided is a compound having a formula, 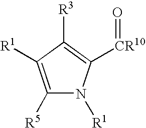
or a pharmaceutically acceptable salt, ester, amide, stereoisomer or prodrug thereof, or a pharmaceutically acceptable salt of the prodrug,
wherein R¹ is lower alkyl, optionally substituted with a halogen;
R³ is benzyl; naphthyl; C₃-C₈ cycloalkyl or C₅-C₈ cycloalkenyl, optionally substituted with one or more heteroatom(s); phenyl or phenyl substituted with one or more groups selected from fluorine, chlorine, bromine, hydroxyl or alkyl of from one to seven carbon atoms; pyridinyl or pyridinyl substituted with fluorine, chlorine, bromine, hydroxyl or alkyl of from one to seven carbon atoms;
R⁴ is H; aryl, aralkyl, heteroaryl or heteroaralkyl; optionally substituted with one or more groups selected from fluorine, chlorine, bromine, hydroxyl or alkyl of from one to seven carbon atoms;
C₁-C₈ alkyl or C₃-C₈ cycloalkyl; optionally substituted; aralkenyl; carbamoyl or substituted carbamoyl; carboxyl or substituted carboxyl;
R⁵ is H, I, phenyl, COOR′, R⁶R⁷NC(O)— or SO₂NR⁶R⁷;
R⁶ and R⁷ are each independently H; aryl, aralkyl, heteroaryl or heteroaralkyl; optionally substituted with halo, alkyl of from one to seven carbon atoms,
—(CH₂)ₙOR′, (CH₂)ₙCOOR′, (CH₂)ₙCONR′R″, (CH₂)ₙS(O)₂NR′R″,
(CH₂)ₙS(O)₂R, or heteroaryl;
C₁-C₁₀ alkyl, C₃-C₈ cycloalkyl or C₅-C₈ cycloalkenyl, said alkyl, cycloalkyl or cycloalkenyl optionally containing one or more heteroatoms(s); unsubstituted or substituted with OH, CO₂R′ or CONR′R″;
COOR′; C(O)R′; SO₂NHR⁸ or SO₂R⁸;
or N, R⁶ and R⁷ taken together form a 4-7 member ring, optionally containing up to 2 heteroatoms selected from O, N and S, said heteroatom(s) being optionally substituted; said ring optionally substituted with lower alkyl, OH, benzyl, phenyl,
CO₂R′ or CONR′R″;
R⁸ is aryl, aralkyl, alkyl, heteroaryl or heteroaralkyl; optionally substituted; R¹⁰ is H, OH, OC₁-C₆ alkyl; R and R are each independently H, C₁-C₁₂ alkyl, aryl, or alkyl or taken together form a 4-7 member ring;
n is 0-2; and
wherein 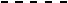
   is a bond or is absent.

Further provided is a compound having a formula, 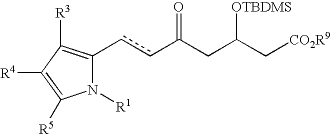
or a pharmaceutically acceptable salt, ester, amide, stereoisomer or prodrug thereof, or a pharmaceutically acceptable salt of the prodrug,
wherein R¹ is lower alkyl, optionally substituted with a halogen;
R³ is benzyl; naphthyl; C₃-C₈ cycloalkyl or C₅-C₈ cycloalkenyl, optionally substituted with one or more heteroatom(s); phenyl or phenyl substituted with one or more groups selected from fluorine, chlorine, bromine, hydroxyl or alkyl of from one to seven carbon atoms; pyridinyl or pyridinyl substituted with fluorine, chlorine, bromine, hydroxyl or alkyl of from one to seven carbon atoms;
R⁴ is H; aryl, aralkyl, heteroaryl or heteroaralkyl; optionally substituted with one or more groups selected from fluorine, chlorine, bromine, hydroxyl or alkyl of from one to seven carbon atoms;
C₁-C₈ alkyl or C₃-C₈ cycloalkyl; optionally substituted; aralkenyl; carbamoyl or substituted carbamoyl; carboxyl or substituted carboxyl;
R⁵ is H, I, phenyl, COOR′, R⁶R⁷NC(O)— or SO₂NR⁶R⁷;
R⁶ and R⁷ are each independently H; aryl, aralkyl, heteroaryl or heteroaralkyl; optionally substituted with halo, alkyl of from one to seven carbon atoms,
(CH₂)ₙOR′, (CH₂)ₙCOOR′, (CH₂)ₙCONR′R″, (CH₂)ₙS(O)₂NR′R″,
(CH₂)ₙS(O)₂R⁸, or heteroaryl;
C₁-C₁₀ alkyl, C₃-C₈ cycloalkyl or C₅-C₈ cycloalkenyl, said alkyl, cycloalkyl or cycloalkenyl optionally containing one or more heteroatoms(s); unsubstituted or substituted with OH, CO₂R′ or CONR′R″;
COOR′; C(O)R′; SO₂NHR⁸ or SO₂R⁸;
or N, R⁶ and R⁷ taken together form a 4-7 member ring, optionally containing up to 2 heteroatoms selected from O, N and S, said heteroatom(s) being optionally substituted; said ring optionally substituted with lower alkyl, OH, benzyl, phenyl,
CO₂R′ or CONR′R″;
R⁸ is aryl, aralkyl, alkyl, heteroaryl or heteroaralkyl; optionally substituted;
R⁹ is C₁-C₆ alkyl or H; and R″ are each independently H, C₁-C₁₂ alkyl, aryl, or alkyl or taken together form a 4-7 member ring;
TBDMS is tert-butyldimethyl-silye-;
n is 0-2; and
wherein 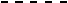
   is a bond or is absent.

Further provided is a compound having a formula, 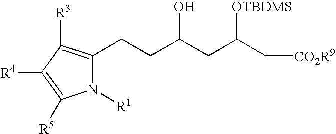
or a pharmaceutically acceptable salt, ester, amide, stereoisomer, racemic mixture or prodrug thereof, or a pharmaceutically acceptable salt of the prodrug, wherein
R¹ is lower alkyl, optionally substituted with a halogen;
R³ is benzyl; naphthyl; C₃-C₈ cycloalkyl or C₅-C₈ cycloalkenyl, optionally substituted with one or more heteroatom(s); phenyl or phenyl substituted with one or more groups selected from fluorine, chlorine, bromine, hydroxyl or alkyl of from one to seven carbon atoms; pyridinyl or pyridinyl substituted with fluorine, chlorine, bromine, hydroxyl or alkyl of from one to seven carbon atoms;
R⁴ is H; aryl, aralkyl, heteroaryl or heteroaralkyl; optionally substituted with one or more groups selected from fluorine, chlorine, bromine, hydroxyl or alkyl of from one to seven carbon atoms;
C₁-C₈ alkyl or C₃-C₈ cycloalkyl; optionally substituted; aralkenyl; carbamoyl or substituted carbamoyl; carboxyl or substituted carboxyl;
R⁵ is H, I, phenyl, COOR′, R⁶R⁷NC(O)— or SO₂NR⁶R⁷;
R⁶ and R⁷ are each independently H; aryl, aralkyl, heteroaryl or heteroaralkyl; optionally substituted with halo, alkyl of from one to seven carbon atoms,
(CH₂)ₙOR′, (CH₂)ₙCOOR′, (CH₂)ₙCONR′R″, (CH₂)ₙS(O)₂NR′R″,
(CH₂)ₙS(O)₂R⁸, or heteroaryl;
C₁-C₁₀ alkyl, C₃-C₈ cycloalkyl or C₅-C₈ cycloalkenyl, said alkyl, cycloalkyl or cycloalkenyl optionally containing one or more heteroatoms(s); unsubstituted or substituted with OH, CO₂R′ or CONR′R″;
COOR′; C(O)R′; SO₂NHR⁸ or SO₂R⁸;
or N, R⁶ and R⁷ taken together form a 4-7 member ring, optionally containing up to 2 heteroatoms selected from O, N and S, said heteroatom(s) being optionally substituted; said ring optionally substituted with lower alkyl, OH, benzyl, phenyl,
CO₂R′ or CONR′R″;
R⁸ is aryl, aralkyl, alkyl, heteroaryl or heteroaralkyl; optionally substituted;
R⁹ is C₁-C₆ alkyl or H; R′ and R″ are each independently H, C₁-C₁₂ alkyl, aryl,
or alkyl or taken together form a 4-7 member ring;
n is 0-2; and
wherein 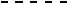
   is a bond or is absent.

Further provided is a compound having a formula, 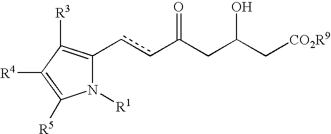
or a pharmaceutically acceptable salt, ester, amide, stereoisomer, racemic mixture or prodrug thereof, or a pharmaceutically acceptable salt of the prodrug, wherein
R¹ is lower alkyl, optionally substituted with a halogen;
R³ is benzyl; naphthyl; C₃-C₈ cycloalkyl or C₅-C₈ cycloalkenyl, optionally substituted with one or more heteroatom(s); phenyl or phenyl substituted with one or more groups selected from fluorine, chlorine, bromine, hydroxyl or alkyl of from one to seven carbon atoms; pyridinyl or pyridinyl substituted with fluorine, chlorine, bromine, hydroxyl or alkyl of from one to seven carbon atoms;
R⁴ is H; aryl, aralkyl, heteroaryl or heteroaralkyl; optionally substituted with one or more groups selected from fluorine, chlorine, bromine, hydroxyl or alkyl of from one to seven carbon atoms;
C₁-C₈ alkyl or C₃-C₈ cycloalkyl; optionally substituted; aralkenyl; carbamoyl or substituted carbamoyl; carboxyl or substituted carboxyl;
R⁵ is H, I, phenyl, COOR′, R⁶R⁷NC(O)— or SO₂NR⁶R⁷;
R⁶ and R⁷ are each independently H; aryl, aralkyl, heteroaryl or heteroaralkyl; optionally substituted with halo, alkyl of from one to seven carbon atoms,
(CH₂)ₙOR′, (CH₂)ₙCOOR′, (CH₂)ₙCONR′R″, (CH₂)ₙS(O)₂NR′R″,
(CH₂)ₙS(O)₂R⁸, or heteroaryl;
C₁-C₁₀-alkyl, C₃-C₈ cycloalkyl or C₅-C₈ cycloalkenyl, said alkyl, cycloalkyl or cycloalkenyl optionally containing one or more heteroatoms(s); unsubstituted or substituted with OH, CO₂R′ or CONR′R″;
COOR′; C(O)R′; SO₂NHR⁸ or SO₂R⁸;
or N, R⁶ and R⁷ taken together form a 4-7 member ring, optionally containing up to 2 heteroatoms selected from O, N and S, said heteroatom(s) being optionally substituted; said ring optionally substituted with lower alkyl, OH, benzyl, phenyl,
CO₂R′ or CONR′R″;
R⁸ is aryl, aralkyl, alkyl, heteroaryl or heteroaralkyl; optionally substituted;
R⁹ is C₁-C₆ alkyl or H; R′ and R″ are each independently H, C₁-C₁₂ alkyl, aryl, or alkyl or taken together form a 4-7 member ring;
n is 0-2; and
wherein 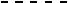
   is a bond or is absent.

Further provided is a compound having a formula 21, 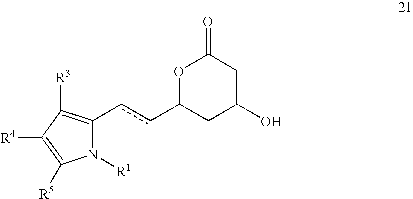
or a pharmaceutically acceptable salt, ester, amide, stereoisomer, racemic mixture or prodrug thereof, or a pharmaceutically acceptable salt of the prodrug, wherein
R¹ is lower alkyl, optionally substituted with a halogen;
R³ is benzyl; naphthyl; C₃-C₈ cycloalkyl or C₅-C₈ cycloalkenyl, optionally substituted with one or more heteroatom(s); phenyl or phenyl substituted with one or more groups selected from fluorine, chlorine, bromine, hydroxyl or alkyl of from one to seven carbon atoms; pyridinyl or pyridinyl substituted with fluorine, chlorine, bromine, hydroxyl or alkyl of from one to seven carbon atoms;
R⁴ is H; aryl, aralkyl, heteroaryl or heteroaralkyl; optionally substituted with one or more groups selected from fluorine, chlorine, bromine, hydroxyl or alkyl of from one to seven carbon atoms;
C₁-C₈ alkyl or C₃-C₈ cycloalkyl; optionally substituted; aralkenyl; carbamoyl or substituted carbamoyl; carboxyl or substituted carboxyl;
R⁵ is H, I, phenyl, COOR′, R⁶R⁷ NC(O)— or SO₂NR⁶R⁷;
R⁶ and R⁷ are each independently H; aryl, aralkyl, heteroaryl or heteroaralkyl; optionally substituted with halo, alkyl of from one to seven carbon atoms,
(CH₂)ₙOR′, (CH₂)ₙCOOR′, (CH₂)ₙCONR′R″, (CH₂)ₙS(O)₂NR′R″,
(CH₂)ₙS(O)₂R⁸, or heteroaryl;
C₁-C₁₀ alkyl, C₃-C₈ cycloalkyl or C₅-C₈ cycloalkenyl, said alkyl, cycloalkyl or cycloalkenyl optionally containing one or more heteroatoms(s); unsubstituted or substituted with OH, CO₂R′ or CONR′R″;
COOR′; C(O)R′; SO₂NHR⁸ or SO₂R⁸;
or N, R⁶ and R⁷ taken together form a 4-7 member ring, optionally containing up to 2 heteroatoms selected from O, N and S, said heteroatom(s) being optionally substituted; said ring optionally substituted with lower alkyl, OH, benzyl, phenyl,
CO₂R′ or CONR′R″;
R⁸ is aryl, aralkyl, alkyl, heteroaryl or heteroaralkyl; optionally substituted;
R′ and R″ are each independently H, C₁-C₁₂ alkyl, aryl, or alkyl or taken together form a 4-7 member ring;
n is 0-2; and wherein 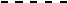
   is a bond or is absent.

Further provided is a compound having a formula, 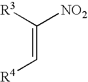
wherein R³ is benzyl; naphthyl; C₃-C₈ cycloalkyl or C₅-C₈ cycloalkenyl, optionally substituted with one or more heteroatom(s); phenyl or phenyl substituted with one or more groups selected from fluorine, chlorine, bromine, hydroxyl or alkyl of from one to seven carbon atoms; pyridinyl or pyridinyl substituted with fluorine, chlorine, bromine, hydroxyl or alkyl of from one to seven carbon atoms; and R⁴ is H; aryl, aralkyl, heteroaryl or heteroaralkyl; optionally substituted with one or more groups selected from fluorine, chlorine, bromine, hydroxyl or alkyl of from one to seven carbon atoms.

Still further provided is a compound selected from the group consisting of:
(4R,6R)-(6-{2-[3,4-Bis-(4-fluoro-phenyl)-1-isopropyl-1H-pyrrol-2-yl)-ethyl}-2,2-dimethyl-[1,3]dioxan-4-yl)-acetic acid;
6-{[5-(6-Carboxy-3,5-dihydroxy-hexyl)-4-(4-fluoro-phenyl)-1-isopropyl-3-phenyl-1H-pyrrole-2-carbonyl]-amino}-nicotinic acid;
7-[5-(Acetylamino-methyl)-3-(4-fluoro-phenyl)-1-isopropyl-4-phenyl-1H-pyrrol-2-yl]-3,5-dihydroxy-heptanoic acid; and pharmaceutically acceptable salts, esters and amides thereof.

### DETAILED DESCRIPTION OF THE INVENTION

The present invention provides a compound having a Formula I, 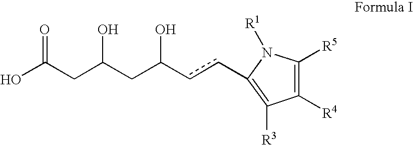
or a pharmaceutically acceptable salt, ester, amide, stereoisomer or prodrug thereof, or a pharmaceutically acceptable salt of the prodrug,
wherein R¹ is lower alkyl, optionally substituted with a halogen;
R³ is benzyl; naphthyl; C₃-C₈ cycloalkyl or C₅-C₈ cycloalkenyl, optionally substituted with one or more heteroatom(s); phenyl or phenyl substituted with one or more groups selected from fluorine, chlorine, bromine, hydroxyl or alkyl of from one to seven carbon atoms; pyridinyl or pyridinyl substituted with fluorine, chlorine, bromine, hydroxyl or alkyl of from one to seven carbon atoms;
R⁴ is H; aryl, aralkyl, heteroaryl or heteroaralkyl; optionally substituted with one or more groups selected from fluorine, chlorine, bromine, hydroxyl or alkyl of from one to seven carbon atoms;
C₁-C₈ alkyl or C₃-C₈ cycloalkyl; optionally substituted; aralkenyl; carbamoyl or substituted carbamoyl; carboxyl or substituted carboxyl;
R⁵ is H, I, phenyl, COOR′, R⁶R⁷NC(O)—, —(CH₂)ₙNR⁶R⁷, or SO₂NR⁶R⁷;
R⁶ and R⁷ are each independently H; aryl, aralkyl, heteroaryl or heteroaralkyl; optionally substituted with halo, alkyl of from one to seven carbon atoms,
(CH₂)ₙOR′, (CH₂)ₙCOOR′, (CH₂)ₙCONR′R″, (CH₂)ₙS(O)₂NR′R″,
(CH₂)ₙS(O)₂R⁸, or heteroaryl;
C₁-C₁₀ alkyl, C₃-C₈ cycloalkyl or C₅-C₈ cycloalkenyl, said alkyl, cycloalkyl or cycloalkenyl optionally containing one or more heteroatoms(s); unsubstituted or substituted with OH, CO₂R′ or CONR′R″; COOR′; C(O)R′; SO₂NHR⁸ or SO₂R⁸;
or N, R⁶ and R⁷ taken together form a 4-7 member ring, optionally containing up to 2 heteroatoms selected from O, N and S, said heteroatom(s) being optionally substituted; said ring optionally substituted with lower alkyl, OH, benzyl, phenyl,
CO₂R′ or CONR′R″;
R⁸ is aryl, aralkyl, alkyl, heteroaryl or heteroaralkyl; optionally substituted;
R′ and R″ are each independently H, C₁-C₁₂ alkyl, aryl, or aralkyl, or taken together form a 4-7 member ring;
n is 0-2; and wherein 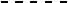
   is a bond or is absent.

Further provided is a stereoisomer of the above-described compound comprising a (3R,5R)-isomer. Further provided is a stereoisomer of the compound comprising a (3R,5S)-isomer. Further provided is a stereoisomer of the compound comprising a (3S,5S)-isomer. Further provided is a stereoisomer of the compound comprising a (3S,5R)-isomer.

Further provided is a compound or the pharmaceutically acceptable salt, ester, amide, stereoisomer or prodrug thereof, or the pharmaceutically acceptable salt of the prodrug, wherein R³ is phenyl or substituted phenyl, or pyridinyl or substituted pyridinyl.

Further provided is a compound or the pharmaceutically acceptable salt, ester, amide, stereoisomer or prodrug thereof, or the pharmaceutically acceptable salt of the prodrug, wherein R³ is phenyl substituted with fluorine, chlorine or bromine. Further provided is the compound wherein R³ is para-fluorophenyl.

Further provided is a compound or the pharmaceutically acceptable salt, ester, amide, stereoisomer or prodrug thereof, or the pharmaceutically acceptable salt of the prodrug, wherein R⁴ is phenyl, biphenyl or substituted phenyl; pyridinyl or substituted pyridinyl; C₁-C₈ alkyl optionally substituted; or naphthyl. Further provided is the compound wherein R⁴ is phenyl or para-fluorophenyl.

Further provided is a compound or the pharmaceutically acceptable salt, ester, amide, stereoisomer or prodrug thereof, or the pharmaceutically acceptable salt of the prodrug, wherein R⁴ is cyclohexyl-, clyclopentyl-, cyclobutyl-, cyclopropyl-, methyl-, ethyl-, isopropyl-, difluoromethyl, trifluoro-methyl or phenyl substituted with one or more halogen.

Further provided is a compound or the pharmaceutically acceptable salt, ester, amide, stereoisomer or prodrug thereof, or the pharmaceutically acceptable salt of the prodrug, wherein R¹ is C₁-C₄ alkyl. Further provided is the compound wherein R¹ is ethyl or propyl. Further provided is the compound wherein R¹ is isopropyl.

Further provided is a compound or the pharmaceutically acceptable salt, ester, amide, stereoisomer or prodrug thereof, or the pharmaceutically acceptable salt of the prodrug, wherein R⁵ is SO₂NR⁶R⁷, —(CH₂)ₙNR⁶R⁷, or R⁶R⁷NC(O)—; R⁴ is phenyl, para-fluorophenyl, isopropyl, cyclopropyl, methyl, ethyl, CHF₂ or CF₃; and R³ is phenyl or para-fluorophenyl.

Further provided is a compound wherein R⁶ and R⁷ are each independently H; methyl; phenyl or phenyl substituted with halo, alkyl of from one to seven carbon atoms, (CH₂)ₙOR′, (CH₂)ₙCOOR′, (CH₂)ₙCONR′R″, (CH₂)ₙS(O)₂NR′R″, (CH₂)ₙS(O)₂R⁸ or heteroaryl; or benzyl or benzyl substituted with halo, alkyl of from one to seven carbon atoms, (CH₂)ₙOR′, (CH₂)ₙCOOR′, (CH₂)ₙCONR′R″, (CH₂)ₙS(O)₂NR′R″, (CH₂)ₙS(O)₂R⁸, or heteroaryl. Further provided is the compound wherein R⁶ and R⁷ are each independently H, phenyl or substituted phenyl, benzyl or substituted benzyl, phenyl-ethyl, pyridinyl or substituted pyridinyl or C₁-C₄ alkyl.

Further provided is a compound or the pharmaceutically acceptable salt, ester, amide, stereoisomer or prodrug thereof, or the pharmaceutically acceptable salt of the prodrug, wherein R¹ is isopropyl, ethyl, trifluoromethyl, difluoromethyl or cyclopropyl. Further provided is a compound wherein R is isopropyl and R is para-fluorophenyl.

Further provided is a sodium salt or a calcium salt of a compound of the invention. Further provided is a methyl ester or ethyl ester of a compound of the invention.

Further provided is a compound or the pharmaceutically acceptable salt, ester, amide, stereoisomer or prodrug thereof, or the pharmaceutically acceptable salt of the prodrug, wherein R⁴ and R³ are each independently phenyl or substituted phenyl and R¹ is C₁-C₄ alkyl. Further provided is the compound wherein R⁵ is SO₂NR⁶R⁷, —(CH₂)ₙNR⁶R⁷, or R⁶R⁷NC(O)—.

Further provided is a compound or the pharmaceutically acceptable salt, ester, amide, stereoisomer or prodrug thereof, or the pharmaceutically acceptable salt of the prodrug, wherein R⁶ and R⁷ are each independently H, Me, phenyl or phenyl substituted with halo alkyl of from one to seven carbon atoms, (CH₂)ₙOR′,
(CH₂)ₙCOOR′, (CH₂)ₙCONR′R″, (CH₂)ₙS(O)₂NR′R″, (CH₂)ₙS(O)₂R⁸, or heteroaryl; or
benzyl or benzyl substituted with halo, alkyl of from one to seven carbon atoms,
(CH₂)ₙOR′, (CH₂)ₙCOOR′, (CH₂)ₙCONR′R″, (CH₂)ₙS(O)₂NR′R″,
(CH₂)ₙS(O)₂R⁸, or heteroaryl.

Further provided is a compound or the pharmaceutically acceptable salt, ester, amide, stereoisomer or prodrug thereof, or the pharmaceutically acceptable salt of the prodrug, wherein at least one of R⁶ or R⁷ is SO₂NHR⁸ or SO₂R⁸ and R⁸ is phenyl or substituted phenyl.

Further provided is a compound wherein N, R⁶ and R⁷ taken together form a 4-7 member ring, optionally containing up to 2 heteroatoms selected from O, N, and S, said up, to 2 heteroatoms being optionally substituted; said ring optionally substituted with lower alkyl, OH, benzyl, phenyl, CO₂R′ or CONR′R″; and R′ and R″ are each independently H, C₁₋₁₂ allkyl, aryl, or taken together form a 4-7 member ring. Further provided is the compound wherein N, R⁶ and R⁷ taken together form a 4-7 member ring, said ring optionally substituted with lower alkyl, phenyl or benzyl.

Further provided is a compound or the pharmaceutically acceptable salt, ester, amide, stereoisomer or prodrug thereof, or the pharmaceutically acceptable salt of the prodrug, wherein R⁴ is carbamoyl substituted with phenyl, said phenyl being optionally substituted with CONR′R″.

Further provided is a compound or the pharmaceutically acceptable salt, ester, amide or prodrug thereof, or the pharmaceutically acceptable salt of the prodrug wherein R¹ is C₂-C₃ alkyl; R³ and R⁴ are each independently phenyl or para-fluorophenyl; and R⁵ is H, I, phenyl, COOR′, R⁶R⁷NC(O)—, —(CH₂)ₙNR⁶R⁷ or SO₂NR⁶R⁷.

Further provided is a pharmaceutical composition comprising a compound of the invention or the pharmaceutically acceptable salt, ester, amide or prodrug thereof, or the pharmaceutically acceptable salt of the prodrug; or a mixture thereof; and a pharmaceutically acceptable carrier, diluent or vehicle.

Further provided is a method of inhibiting cholesterol biosynthesis in a mammal requiring inhibition comprising administering to the mammal a therapeutically effective amount of a compound of the invention or the pharmaceutically acceptable salt, ester, amide or prodrug thereof, or the pharmaceutically acceptable salt of the prodrug. Further provided is a method of lowering LDL cholesterol in a mammal. Further provided is a method of raising HDL cholesterol in a mammal.

Further provided is a method of treating, preventing or controlling hyperlipidemia in a mammal comprising administering to the mammal in need thereof a therapeutically effective amount of a compound of the invention or the pharmaceutically acceptable salt, ester, amide or prodrug thereof, or the pharmaceutically acceptable salt of the prodrug. Further provided is a method of treating, preventing or controlling hypercholesterolemia in a mammal. Further provided is a method of treating, preventing or controlling hypertriglyceridemia in a mammal.

Further provided is a method of treating, preventing or controlling atherosclerosis in a mammal comprising administering to the mammal in need thereof a therapeutically effective amount of a compound of the invention or the pharmaceutically acceptable salt, ester, amide or prodrug thereof, or the pharmaceutically acceptable salt of the prodrug. Further provided is a method of treating, preventing or controlling Alzheimer's disease, benign prostatic hyperplasia (“BPH”), diabetes or osteoporosis in a mammal.

The present invention provides inter alia the following compounds:
(3R,5R)-7-[3-(4-fluoro-phenyl)-1-isopropyl-4-phenyl-5-phenylcarbamoyl-1H-pyrrol-2-yl]-3,5-dihydroxy-heptanoic acid;
(3R,5S)-7-[3-(4-Fluoro-phenyl)-1-isopropyl-4-phenyl-5-phenylcarbamoyl-1H-pyrrol-2-yl]-3,5-dihydroxy-hept-6-enoic acid;
(3R)-7-[3-(4-Fluoro-phenyl)-1-isopropyl-4-phenyl-5-phenylcarbamoyl-1H-pyrrol-2-yl]-3,5-dihydroxy-heptanoic acid;
(3R,5R)-7-[3-(4-Fluoro-phenyl)-1-isopropyl-4-phenyl-5-(4-sulfamoyl-phenylcarbamoyl)-1H-pyrrol-2-yl]-3,5-dihydroxy-heptanoic acid;
(3R,5S)-7-[3-(4-Fluoro-phenyl)-1-isopropyl-4-phenyl-5-(4-sulfamoyl-phenylcarbamoyl)-1H-pyrrol-2-yl]-3,5-dihydroxy-hept-6-enoic acid;
(3R,5R)-7-[3-(4-Fluoro-phenyl)-5-(4-fluoro-phenylcarbamoyl)-1-isopropyl-4-phenyl-1H-pyrrol-2-yl]-3,5-dihydroxy-heptanoic acid;
(3R,5S)-7-[3-(4-Fluoro-phenyl)-5-(4-fluoro-phenylcarbamoyl)-1-isopropyl-4-phenyl-5-phenylcarbamoyl-1H-pyrrol-2-yl]-3,5-dihydroxy-hept-6-enoic acid;
(3R)-7-[3-(4-Fluoro-phenyl)-5-(4-fluoro-phenylcarbamoyl)-1-isopropyl-4-phenyl-1H-pyrrol-2-yl]-3,5-dihydroxy-heptanoic acid;
(3R,5R)-7-[5-(4-Fluoro-benzylcarbamoyl)-3-(4-fluoro-phenyl)-1-isopropyl-4-phenyl-1H-pyrrol-2-yl]-3,5-dihydroxy-heptanoic acid;
(3R,5S)-7-[5-(4-Fluoro-benzylcarbamoyl)-3-(4-fluoro-phenyl)-1-isopropyl-4-phenyl-1H-pyrrol-2-yl]-3,5-dihydroxy-hept-6-enoic acid;
(3R,5R)-7-[3,4-Bis-(4-fluoro-phenyl)-1-isopropyl-5-(4-sulfamoyl-phenylcarbamoyl)-1H-pyrrol-2-yl]-3,5-dihydroxy-heptanoic acid;
(3R,5S)-7-[3,4-Bis-(4-fluoro-phenyl)-1-isopropyl-5-(4-sulfamoyl-phenycarbamoyl-1H-pyrrol-2-yl]-3,5-dihydroxy-hept-6-enoic acid;
(3R,5R)-7-[5-(4-Carbamoylmethyl-phenylcarbamoyl)-3,4-bis-(4-fluoro-phenyl)-1-isopropyl-)-1H-pyrrol-2-yl]-3,5-dihydroxy-heptanoic acid;
(3R,5S)-7-[5-(4-Carbamoylmethyl-phenylcarbamoyl)-3,4-bis-(4-fluoro-phenyl)-1-isopropyl-1H-pyrrol-2-yl]-3,5-dihydroxy-hept-6-enoic acid;
(3R,5R)-7-[3,4-bis(4-fluorophenyl)-1-isopropyl-5-phenylcarbamoyl-1H-pyrrol-2-yl]-3,5-dihydroxy-heptanoic acid;
(3R,5R)-7-[3,4-bis(4-fluorophenyl)-5-(2-fluoro-phenylcarbamoyl)-1-isopropyl-1H-pyrrol-2-yl]-3,5-dihydroxy-heptanoic acid;
(3R,5S)-7-[3,4-bis(4-fluorophenyl)-5-(4-fluorophenylcarbamoyl)-1-isopropyl-1H-pyrrol-2-yl]-3,5-dihydroxy-hept-6-enoic acid;
(3R,5R)-7-[5-(2,4-difluoro-phenylcarbamoyl)-3,4-bis(4-fluoro-phenyl)-1-isopropyl-1H-pyrrol-2-yl]-3,5-dihydroxy-heptanoic acid;
(3R,5R)-7-[3,4-bis-(4-fluorophenyl)-1-isopropyl-5-p-tolylcarbamoyl-1H-pyrrol-2-yl]-3,5-dihydroxy-heptanoic acid;
(3R,5R)-7-[3,4-bis(4-fluorophenyl)-1-isopropyl-5-m-tolylcarbamoyl-1H-pyrrol-2-yl]-3,5-dihydroxy-heptanoic acid;
(3R,5R)-7-[1-Ethyl-3-(4-fluoro-phenyl)-4-isopropyl-5-phenylcarbamoyl-1H-pyrrol-2-yl]-3,5-dihydroxy-heptanoic acid;
(3R,5R)-7-[1-ethyl-3-(4-fluoro-phenyl)-4-methyl-5-phenylcarbamoyl-1-Hpyrrol-2-yl]-3,5-dihydroxy-heptanoic acid;
(3R,5R)-7-[3,4-bis(4-fluorophenyl)-1-isopropyl-5-(piperidine-1-carbonyl)-1H-pyrrol-2-yl]-3,5-dihydroxy-heptanoic acid;
(3R,5R)-7-[3,4-Bis-(4-fluoro-phenyl)-1-isopropyl-5-(4-methanesulfonyl-benzylcarbamoyl)-1H-pyrrol-2-yl]-3,5-dihydroxy-heptanoic acid;
(3R,5R)-7-[3,4-Bis-(4-fluoro-phenyl)-1-isopropyl-5-(4-sulfamoyl-benzylcarbamoyl)-1H-pyrrol-2-yl]-3,5-dihydroxy-heptanoic acid;
trans-(3R,5S)-7-[3,4-Bis-(4-fluoro-phenyl)-1-isopropyl-5-(4-sulfamoyl-benzylcarbamoyl)-1H-pyrrol-2-yl]-3,5-dihydroxy-hept-6-enoic acid;
(3R,5R)-7-[5-(4-Dimethylcarbamoyl-benzylcarbamoyl)-3,4-bis-(4-fluoro-phenyl)-1-isopropyl-1H-pyrrol-2-yl]-3,5-dihydroxy-heptanoic acid;
(3R,5R)-7-[3,4-Bis-(4-fluoro-phenyl)-1-isopropyl-5-(4-sulfamoylmethyl-phenylcarbamoyl)-1H-pyrrol-2-yl]-3,5-dihydroxy-heptanoic acid;
trans-(3R,5S)-7-[3,4-Bis-(4-fluoro-phenyl)-1-isopropyl-5-(4-sulfamoylmethyl-phenylcarbamoyl)-1H-pyrrol-2-yl]-3,5-dihydroxy-hept-6-enoic acid;
(3R,5R)-7-[3,4-Bis-(4-fluoro-phenyl)-1-isopropyl-5-(4-methanesulfonylmethyl-phenylcarbamoyl)-1H-pyrrol-2-yl]-3,5-dihydroxy-heptanoic acid;
trans-(3R,5S)-7-[3,4-Bis-(4-fluoro-phenyl)-1-isopropyl-5-(4-methanesulfonylmethyl-phenylcarbamoyl)-1H-pyrrol-2-yl]-3,5-dihydroxy-hept-6-enoic acid;
(3R,5R)-7-{3-(4-Fluoro-phenyl)-1-isopropyl-4-phenyl-5-[(pyridin-2-ylmethyl)-carbamoyl]-1H-pyrrol-2-yl}-3,5-dihydroxy-heptanoic acid;
trans-(3R,5S)-7-{3-(4-Fluoro-phenyl)-1-isopropyl-4-phenyl-5-[(pyridin-2-ylmethyl)-carbamoyl]-1H-pyrrol-2-yl}-3,5-dihydroxy-hept-6-enoic acid;
(3R,5R)-7-[5-(3-Dimethylcarbamoyl-phenylcarbamoyl)-3-(4-fluoro-phenyl)-1-isopropyl-4-phenyl-1H-pyrrol-2-yl]-3,5-dihydroxy-heptanoic acid;
trans-(3R,5S)-7-[5-(3-Dimethylcarbamoyl-phenylcarbamoyl)-3-(4-fluoro-phenyl)-1-isopropyl-4-phenyl-1H-pyrrol-2-yl]-3,5-dihydroxy-hept-6-enoic acid;
(3R,5R)-3-{[5-(6-Carboxy-3,5-dihydroxy-hexyl)-4-(4-fluoro-phenyl)-1-isopropyl-3-phenyl-1H-pyrrole-2-carbonyl]-amino}-benzoic acid methyl ester;
(3R,5R)-3-{[5-(6-Carboxy-3,5-dihydroxy-hexyl)-4-(4-fluoro-phenyl)-1-isopropyl-3-phenyl-1H-pyrrole-2-carbonyl]-amino 1-benzoic acid;
trans-(3R,5S)-3-{[5-(6-Carboxy-3,5-dihydroxy-hex-1-enyl)-4-(4-fluoro-phenyl)-1-isopropyl-3-phenyl-1H-pyrrole-2-carbonyl]-amino}-benzoic acid methyl ester;
trans-(3R,5S)-3-{[5-(6-Carboxy-3,5-dihydroxy-hex-1-enyl)-4-(4-fluoro-phenyl)-1-isopropyl-3-phenyl-1H-pyrrole-2-carbonyl]-amino}-benzoic acid;
(3R,5R)-7-[37(4-Fluoro-phenyl)-1-isopropyl-5-(5-methyl-isoxazol-3-ylcarbamoyl)-4-phenyl-1H-pyrrol-2-yl]-3,5-dihydroxy-heptanoic acid;
trans-(3R,5S)-7-[3-(4-Fluoro-phenyl)-1-isopropyl-5-(5-methyl-isoxazol-3-ylcarbamoyl)-4-phenyl-1H-pyrrol-2-yl]-3,5-dihydroxy-hept-6-enoic acid;
(3R,5R)-7-[3-(4-Fluoro-phenyl)-1-isopropyl-5-(4-methyl-pyrimidin-2-ylcarbamoyl)-4-phenyl-1H-pyrrol-2-yl]-3,5-dihydroxy-heptanoic acid;
trans-(3R,5S)-7-[3-(4-Fluoro-phenyl)-1-isopropyl-5-(4-methyl-pyrimidin-2-ylcarbamoyl)-4-phenyl-1H-pyrrol-2-yl]-3,5-dihydroxy-hept-6-enoic acid;
(3R,5R)-7-[3-(4-Fluoro-phenyl)-1-isopropyl-5-(3-oxazol-2-yl-phenylcarbamoyl)-4-phenyl-1H-pyrrol-2-yl]-3,5-dihydroxy-heptanoic acid;
trans-(3R,5S)-7-[3-(4-Fluoro-phenyl)-1-isopropyl-5-(3-oxazol-2-yl-phenylcarbamoyl)-4-phenyl-1H-pyrrol-2-yl]-3,5-dihydroxy-hept-6-enoic acid;
(3R,5R)-7-[3-(4-Fluoro-phenyl)-1-isopropyl-5-(4-oxazol-2-yl-phenylcarbamoyl)-4-phenyl-1H-pyrrol-2-yl]-3,5-dihydroxy-heptanoic acid;
trans-(3R,5S)-7-[3-(4-Fluoro-phenyl)-1-isopropyl-5-(4-oxazol-2-yl-phenylcarbamoyl)-4-phenyl-1H-pyrrol-2-yl]-3,5-dihydroxy-hept-6-enoic acid;
(3R,5R)-7-[3-(4-Fluoro-phenyl)-1-isopropyl-4-phenyl-5-(pyrimidin-2-ylcarbamoyl)-1H-pyrrol-2-yl]-3,5-dihydroxy-heptanoic acid;
trans-(3R,5S)-7-[3-(4-Fluoro-phenyl)-1-isopropyl-4-phenyl-5-(pyrimidin-2-ylcarbamoyl)-1H-pyrrol-2-yl]-3,5-dihydroxy-hept-6-enoic acid;
trans-(3R,5S)-7-[5-(4-Dimethylcarbamoyl-phenylcarbamoyl)-3,4-bis-(4-fluoro-phenyl)-1-isopropyl-1H-pyrrol-2-yl]-3,5-dihydroxy-hept-6-enoic acid;
(3R,5R)-7-[5-(4-Dimethylcarbamoyl-phenylcarbamoyl)-3,4-bis-(4-fluoro-phenyl)-1-isopropyl-1H-pyrrol-2-yl]-3,5-dihydroxy-heptanoic acid;
(3R)-7-[5-(4-Dimethylcarbamoyl-phenylcarbamoyl)-3,4-bis-(4-fluoro-phenyl)-1-isopropyl-1H-pyrrol-2-yl]-3-hydroxy-heptanoic acid;
trans-(3R,5S)-7-[5-(4-Dimethylsulfamoyl-benzylcarbamoyl)-3,4-bis-(4-fluoro-phenyl)-1-isopropyl-1H-pyrrol-2-yl]-3,5-dihydroxy-hept-6-enoic acid;
(3R,5R)-7-[5-(4-Dimethylsulfamoyl-benzylcarbamoyl)-3,4-bis-(4-fluoro-phenyl)-1-isopropyl-1H-pyrrol-2-yl]-3,5-dihydroxy-heptanoic acid;
trans-(3R,5S)-7-{3,4-Bis-(4-fluoro-phenyl)-1-isopropyl-5-[(pyridin-2-ylmethyl)-carbamoyl]-1H-pyrrol-2-yl}-3,5-dihydroxy-hept-6-enoic acid;
(3R,5R)-7-{3,4-Bis-(4-fluoro-phenyl)-1-isopropyl-5-[(pyridin-2-ylmethyl)-carbamoyl]-1H-pyrrol-2-yl}-3,5-dihydroxy-heptanoic acid;
trans-(3R,5S)-7-[5-(3-Diemethylsulfamoyl-benzylcarbamoyl)-3,4-bis-(4-fluoro-phenyl)-1-isopropyl-1H-pyrrol-2-yl]-3,5-dihydroxy-hept-6-enoic acid;
(3R,5R)-7-[5-(3-Diemethylsulfamoyl-benzylcarbamoyl)-3,4-bis-(4-fluoro-phenyl)-1-isopropyl-1H-pyrrol-2-yl]-3,5-dihydroxy-heptanoic acid;
trans-(3R,5S)-7-[5-(3-Dimethylcarbamoyl-phenylcarbamoyl)-3,4-bis-(4-fluoro-phenyl)-1-isopropyl-1H-pyrrol-2-yl]-3,5-dihydroxy-hept-6-enoic acid;
(3R,5R)-7-[5-(3-Dimethylcarbamoyl-phenylcarbamoyl)-3,4-bis-(4-fluoro-phenyl)-1-isopropyl-1H-pyrrol-2-yl]-3,5-dihydroxy-heptanoic acid;
trans-(3R,5S)-{[5-(6-Carboxy-3,5-dihydroxy-hex-1-enyl)-3,4-bis-(4-fluoro-phenyl)-1-isopropyl-1H-pyrrole-2-carbonyl]-amino}-benzoic acid methyl ester;
(3R,5R)-{[5-(6-Carboxy-3,5-dihydroxy-hexyl)-3,4-bis-(4-fluoro-phenyl)-1-isopropyl-1H-pyrrole-2-carbonyl]-amino}-benzoic acid methyl ester;
trans-(3R,5S)-7-[3,4-Bis-(4-fluoro-phenyl)-1-isopropyl-5-(4-methyl-pyrimidin-2-ylcarbamoyl)-1H-pyrrol-2-yl]-3,5-dihydroxy-hept-6-enoic acid;
(3R,5R)-7-[3,4-Bis-(4-fluoro-phenyl)-1-isopropyl-5-(4-methyl-pyrimidin-2-ylcarbamoyl)-1H-pyrrol-2-yl]-3,5-dihydroxy-heptanoic acid;
trans-(3R,5S)-7-[3,4-Bis-(4-fluoro-phenyl)-1-isopropyl-5-(4-oxazol-2-yl-phenylcarbamoyl)-1H-pyrrol-2-yl]-3,5-dihydroxy-hept-6-enoic acid;
(3R,5R)-7-[3,4-Bis-(4-fluoro-phenyl)-1-isopropyl-5-(4-oxazol-2-yl-phenylcarbamoyl)-1H-pyrrol-2-yl]-3,5-dihydroxy-heptanoic acid;
trans-(3R,5S)-7-[3,4-Bis-(4-fluoro-phenyl)-1-isopropyl-5-(3-oxazol-2-yl-phenylcarbamoyl)-1H-pyrrol-2-yl]-3,5-dihydroxy-hept-6-enoic acid;
(3R,5R)-7-[3,4-Bis-(4-fluoro-phenyl)-1-isopropyl-5-(3-oxazol-2-yl-phenylcarbamoyl)-1H-pyrrol-2-yl]-3,5-dihydroxy-heptanoic acid;
trans-(3R,5S)-7-[3,4-Bis-(4-fluoro-phenyl)-1-isopropyl-5-(5-methyl-isoxazol-3-ylcarbamoyl)-1H-pyrrole-2-yl]-3,5-dihydroxy-hept-6-enoic acid;
(3R,5R)-7-[3,4-Bis-(4-fluoro-phenyl)-1-isopropyl-5-(5-methyl-isoxazol-3-ylcarbamoyl)-1H-pyrrole-2-yl]-3,5-dihydroxy-heptanoic acid;
(3R,5S)-7-[5-(4-Benzyloxy-phenylcarbamoyl)-3,4-bis-(4-fluoro-phenyl)-1-isopropyl-1H-pyrrol-2-yl]-3,5-dihydroxy-hept-6-enoic acid;
(3R,5S)-7-[3,4-Bis-(4-fluoro-phenyl)-1-isopropyl-5-(4-methoxycarbonyl-benzylcarbamoyl)-1H-pyrrol-2-yl]-3,5-dihydroxy-hept-6-enoic acid;
(3R,5R)-7-[3,4-Bis-(4-fluoro-phenyl)-5-(4-hydroxy-phenylcarbamoyl)-1-isopropyl-1H-pyrrol-2-yl]-3,5-dihydroxy-heptanoic acid;
(3R,5R)-7-[5-(4-Benzyloxy-phenylcarbamoyl)-3,4-bis-(4-fluoro-phenyl)-1-isopropyl-1H-pyrrol-2-yl]-3,5-dihydroxy-heptanoic acid;
(3R,5R)-7-[3,4-Bis-(4-fluoro-phenyl)-1-isopropyl-5-(4-methoxycarbonyl-benzylcarbamoyl)-1H-pyrrol-2-yl]-3,5-dihydroxy-heptanoic acid;
(3R,5S)-7-[5-(2-Benzyloxy-phenylcarbamoyl)-3,4-bis-(4-fluoro-phenyl)-1-isopropyl-1H-pyrrol-2-yl]-3,5-dihydroxy-hept-6-enoic acid;
(3R,5R)-7-[5-(4-Carboxy-benzylcarbamoyl)-3,4-bis-(4-fluoro-phenyl)-1-isopropyl-1H-pyrrol-2-yl]-3,5-dihydroxy-heptanoic acid;
(3R,5S)-7-[5-(3-Benzyloxy-phenylcarbamoyl)-3,4-bis-(4-fluoro-phenyl)-1-isopropyl-1H-pyrrol-2-yl]-3,5-dihydroxy-hept-6-enoic acid;
(3R,5S)-7-[3,4-Bis-(4-fluoro-phenyl)-1-isopropyl-5-(3-sulfamoyl-phenylcarbamoyl)-1H-pyrrol-2-yl]-3,5-dihydroxy-hept-6-enoic acid;
(3R,5R)-7-[3,4-Bis-(4-fluoro-phenyl)-5-(3-hydroxy-phenylcarbamoyl)-1-isopropyl-1H-pyrrol-2-yl]-3,5-dihydroxy-heptanoic acid;
(3R,5R)-7-[3,4-Bis-(4-fluoro-phenyl)-5-(2-hydroxy-phenylcarbamoyl)-1-isopropyl-1H-pyrrol-2-yl]-3,5-dihydroxy-heptanoic acid;
(3R,5S)-7-[3,4-Bis-(4-fluoro-phenyl)-1-isopropyl-5-(3-methoxy-phenylcarbamoyl)-1H-pyrrol-2-yl]-3,5-dihydroxy-hept-6-enoic acid;
(3R,5R)-7-[3,4-Bis-(4-fluoro-phenyl)-1-isopropyl-5-(3-sulfamoyl-phenylcarbamoyl)-1H-pyrrol-2-yl]-3,5-dihydroxy-heptanoic acid;
(3R,5R)-7-[3,4-Bis-(4-fluoro-phenyl)-1-isopropyl-5-(3-methoxy-phenylcarbamoyl)-1H-pyrrol-2-yl]-3,5-dihydroxy-heptanoic acid;
(3R,5S)-7-[5-(3-Chloro-phenylcarbamoyl)-3,4-bis-(4-fluoro-phenyl)-1-isopropyl-1H-pyrrol-2-yl]-3,5-dihydroxy-hept-6-enoic acid;
(3R,5S)-7-[5-(3-Ethyl-phenylcarbamoyl)-3,4-bis-(4-fluoro-phenyl)-1-isopropyl-1H-pyrrol-2-yl]-3,5-dihydroxy-hept-6-enoic acid;
(3R,5R)-7-[5-(3-Ethyl-phenylcarbamoyl)-3,4-bis-(4-fluoro-phenyl)-1-isopropyl-1H-pyrrol-2-yl]-3,5-dihydroxy-heptanoic acid;
(3R,5R)-7-[5-(3-Chloro-phenylcarbamoyl)-3,4-bis-(4-fluoro-phenyl)-1-isopropyl-1H-pyrrol-2-yl]-3,5-dihydroxy-heptanoic acid;
(3R,5R)-7-[3,4-bis(4-fluorophenyl)-5-(4-fluoro-phenylcarbamoyl)-1-isopropyl-1H-pyrrol-2-yl]-3,5-dihydroxy-heptanoic acid;
(3R,5R)-7-[3,4-bis(4-fluoro-phenyl)-5-(3-fluorophenylcarbamoyl)-1-isopropyl-1H-pyrrol-2-yl]-3,5-dihydroxy-heptanoic acid;
(3R,5R)-7-[5-(4-Carboxymethyl-phenylcarbamoyl)-3,4-bis(4-fluorophenyl)-1-isopropyl-1H-pyrrol-2-yl]-3,5-dihydroxy-heptanoic acid;
(3R,5R)-7-[5-(4-ethylpiperazine-1-carbonyl)-3,4-bis(4-fluorophenyl)-1-isopropyl-1H-pyrrol-2-yl]-3,5-dihydroxy-heptanoic acid;
(3R,5R)-7-[5-(4-carbamoyl-phenylcarbamoyl)-3,4-bis(4-fluorophenyl)-1-isopropyl-1H-pyrrol-2-yl]-3,5-dihydroxy-heptanoic acid;
(3R,5R)-7-[3,4-bis(4-fluorophenyl)-1-isopropyl-5-(47-methoxycarbonyl-phenylcarbamoyl)-1H-pyrrol-2-yl]-3,5-dihydroxy-heptanoic acid;
(3R,5R)-7-[3,4-bis-(4-fluorophenyl)-1-isopropyl-5-(4-sulfamoyl-phenylcarbamoyl)-1H-pyrrol-2-yl]-3,5-dihydroxy-heptanoic acid;
(3R,5R)-7-[5-(3,5-difluorophenylcarbamoyl)-3-(4-fluorophenyl)-1-isopropyl-4-phenyl-1H-pyrrol-2-yl]-3,5-dihydroxy-heptanoic acid;
(3R,5R)-7-[3-(4-fluoro-phenyl)-1-isopropyl-4-phenyl-5-(pyridin-2-ylcarbamoyl)-1H-pyrrol-2-yl]-3,5-dihydroxy-heptanoic acid;
   and pharmaceutically acceptable salts, esters and amides thereof.

The present invention provides a racemic mixture comprising a compound of the invention.

Further provided is a compound having a formula, 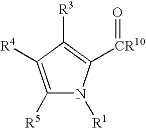
or a pharmaceutically acceptable salt, ester, amide, stereoisomer or prodrug thereof, or a pharmaceutically acceptable salt of the prodrug,
wherein R¹ is lower alkyl, optionally substituted with a halogen;
R³ is benzyl; naphthyl; C₃-C₈ cycloalkyl or C₅-C₈ cycloalkenyl, optionally substituted with one or more heteroatom(s); phenyl or phenyl substituted with one or more groups selected from fluorine, chlorine, bromine, hydroxyl or alkyl of from one to seven carbon atoms; pyridinyl or pyridinyl substituted with fluorine, chlorine, bromine, hydroxyl or alkyl of from one to seven carbon atoms;
R⁴ is H; aryl, aralkyl, heteroaryl or heteroaralkyl; optionally substituted with one or more groups selected from fluorine, chlorine, bromine, hydroxyl or alkyl of from one to seven carbon atoms;
C₁-C₈ alkyl or C₃-C₈ cycloalkyl; optionally substituted; aralkenyl; carbamoyl or substituted carbamoyl; carboxyl or substituted carboxyl;
R⁵ is H, I, phenyl, COOR′, R⁶R⁷NC(O)— or SO₂NR⁶R⁷;
R⁶ and R⁷ are each independently H; aryl, aralkyl, heteroaryl or heteroaralkyl; optionally substituted with halo, alkyl of from one to seven carbon atoms,
(CH₂)ₙOR′, (CH₂)ₙCOOR′, (CH₂)ₙCONR′R″, (CH₂)ₙS(O)₂NR′R″,
(CH₂)ₙS(O)₂R⁸, or heteroaryl;
C₁-C₁₀ alkyl, C₃-C₈ cycloalkyl or C₅-C₈ cycloalkenyl, said alkyl, cycloalkyl or cycloalkenyl optionally containing one or more heteroatoms(s); unsubstituted or substituted with OH, CO₂R′ or CONR′R″;
COOR′; C(O)R′; SO₂NHR⁸ or SO₂R⁸;
or N, R⁶ and R⁷ taken together form a 4-7 member ring, optionally containing up to 2 heteroatoms selected from O, N and S, said heteroatom(s) being optionally substituted; said ring optionally substituted with lower alkyl, OH, benzyl, phenyl, CO₂R′ or CONR′R″;
R⁸ is aryl, aralkyl, alkyl, heteroaryl or heteroaralkyl; optionally substituted; R¹⁰ is H, OH, OC₁-C₆ alkyl; R′ and R″ are each independently H, C₁-C₁₂ alkyl, aryl, or alkyl or taken together form a 4-7 member ring;
n is 0-2; and
wherein 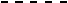
   is a bond or is absent.

Further provided is a compound having a formula, 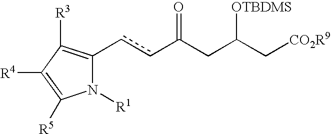
or a pharmaceutically acceptable salt, ester, amide, stereoisomer or prodrug thereof, or a pharmaceutically acceptable salt of the prodrug,
wherein R¹ is lower alkyl, optionally substituted with a halogen;
R³ is benzyl; naphthyl; C₃-C₈ cycloalkyl or C₅-C₈ cycloalkenyl, optionally substituted with one or more heteroatom(s); phenyl or phenyl substituted with one or more groups selected from fluorine, chlorine, bromine, hydroxyl or alkyl of from one to seven carbon atoms; pyridinyl or pyridinyl substituted with fluorine, chlorine, bromine, hydroxyl or alkyl of from one to seven carbon atoms;
R⁴ is H; aryl, aralkyl, heteroaryl or heteroaralkyl; optionally substituted with one or more groups selected from fluorine, chlorine, bromine, hydroxyl or alkyl of from one to seven carbon atoms;
C₁-C₈ alkyl or C₃-C₈ cycloalkyl; optionally substituted; aralkenyl; carbamoyl or substituted carbamoyl; carboxyl or substituted carboxyl;
R⁵ is H, I, phenyl, COOR′, R⁶R⁷NC(O)— or SO₂NR⁶R⁷;
R⁶ and R⁷ are each independently H; aryl, aralkyl, heteroaryl or heteroaralkyl; optionally substituted with halo, alkyl of from one to seven carbon atoms,
(CH₂)ₙOR′, (CH₂)ₙCOOR′, (CH₂)ₙCONR′R″, (CH₂)ₙS(O)₂NR′R″,
(CH₂)ₙS(O)₂R⁸, or heteroaryl;
C₁-C₁₀ alkyl, C₃-C₈ cycloalkyl or C₅-C₈ cycloalkenyl, said alkyl, cycloalkyl or cycloalkenyl optionally containing one or more heteroatoms(s); unsubstituted or substituted with OH, CO₂R′ or CONR′R″;
COOR′; C(O)R′; SO₂NHR⁸ or SO₂R⁸;
or N, R⁶ and R⁷ taken together form a 4-7 member ring, optionally containing up to 2 heteroatoms selected from O, N and S, said heteroatom(s) being optionally substituted; said ring optionally substituted with lower alkyl, OH, benzyl, phenyl,
CO₂R′ or CONR′R″;
R⁸ is aryl, aralkyl, alkyl, heteroaryl or heteroaralkyl; optionally substituted;
R⁹ is C₁-C₆ alkyl or H; and R″ are each independently H, C₁-C₁₂ alkyl, aryl, or alkyl or taken together form a 4-7 member ring; TBDMS is tert-butyldimethyl-silye-;
n is 0-2; and
wherein 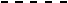
   is a bond or is absent.

Further provided is a compound having a formula, 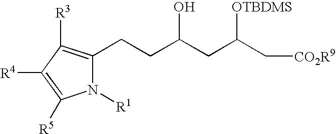
or a pharmaceutically acceptable salt, ester, amide, stereoisomer, racemic mixture or prodrug thereof, or a pharmaceutically acceptable salt of the prodrug, wherein
R¹ is lower alkyl, optionally substituted with a halogen;
R³ is benzyl; naphthyl; C₃-C₈ cycloalkyl or C₅-C₈ cycloalkenyl, optionally substituted with one or more heteroatom(s); phenyl or phenyl substituted with one or more groups selected from fluorine, chlorine, bromine, hydroxyl or alkyl of from one to seven carbon atoms; pyridinyl or pyridinyl substituted with fluorine, chlorine, bromine, hydroxyl or alkyl of from one to seven carbon atoms;
R⁴ is H; aryl, aralkyl, heteroaryl or heteroaralkyl; optionally substituted with one or more groups selected from fluorine, chlorine, bromine, hydroxyl or alkyl of from one to seven carbon atoms;
C₁-C₈ alkyl or C₃-C₈ cycloalkyl; optionally substituted; aralkenyl; carbamoyl or substituted carbamoyl; carboxyl or substituted carboxyl;
R⁵ is H, I, phenyl, COOR′, R⁶R⁷NC(O)— or SO₂NR⁶R⁷;
R⁶ and R⁷ are each independently H; aryl, aralkyl, heteroaryl or heteroaralkyl; optionally substituted with halo, alkyl of from one to seven carbon atoms,
(CH₂)ₙOR′, (CH₂)ₙCOOR′, (CH₂)ₙCONR′R″, (CH₂)ₙS(O)₂NR′R″,
(CH₂)ₙS(O)₂R⁸, or heteroaryl;
C₁-C₁₀ alkyl, C₃-C₈ cycloalkyl or C₅-C₈ cycloalkenyl, said alkyl, cycloalkyl or cycloalkenyl optionally containing one or more heteroatoms(s); unsubstituted or substituted with OH, CO₂R′or CONR′R″;
COOR′; C(O)R′; SO₂NHR⁸ or SO₂R⁸;
or N, R⁶ and R⁷ taken together form a 4-7 member ring, optionally containing up to 2 heteroatoms selected from O, N and S, said heteroatom(s) being optionally substituted; said ring optionally substituted with lower alkyl, OH, benzyl, phenyl,
CO₂R′ or CONR′R″;
R⁸ is aryl, aralkyl, alkyl, heteroaryl or heteroaralkyl; optionally substituted;
R⁹ is C₁-C₆ alkyl or H; R′ and R″ are each independently H, C₁-C₁₂ alkyl, aryl,
or alkyl or taken together form a 4-7 member ring;
n is 0-2; and
wherein 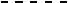
   is a bond or is absent.

Further provided is the compound having a Formula 19, 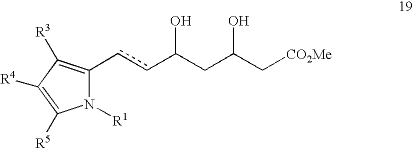
or a pharmaceutically acceptable salt, ester, amide, stereoisomer, racemic mixture or prodrug thereof, or a pharmaceutically acceptable salt of the prodrug.

Further provided is a compound having a formula, 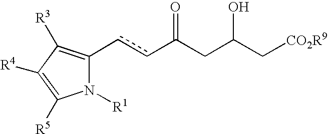
or a pharmaceutically acceptable salt, ester, amide, stereoisomer, racemic mixture or prodrug thereof, or a pharmaceutically acceptable salt of the prodrug, wherein
R¹ is lower alkyl, optionally substituted with a halogen;
R³ is benzyl; naphthyl; C₃-C₈ cycloalkyl or C₅-C₈ cycloalkenyl, optionally substituted with one or more heteroatom(s); phenyl or phenyl substituted with one or more groups selected from fluorine, chlorine, bromine, hydroxyl or alkyl of from one to seven carbon atoms; pyridinyl or pyridinyl substituted with fluorine, chlorine, bromine, hydroxyl or alkyl of from one to seven carbon atoms;
R⁴ is H; aryl, aralkyl, heteroaryl or heteroaralkyl; optionally substituted with one or more groups selected from fluorine, chlorine, bromine, hydroxyl or alkyl of from one to seven carbon atoms;
C₁-C₈ alkyl or C₃-C₈ cycloalkyl; optionally substituted; aralkenyl; carbamoyl or substituted carbamoyl; carboxyl or substituted carboxyl;
R⁵ is H, I, phenyl, COOR′, R⁶R⁷NC(O)— or SO₂NR⁶R⁷;
R⁶ and R⁷ are each independently H; aryl, aralkyl, heteroaryl or heteroaralkyl; optionally substituted with halo, alkyl of from one to seven carbon atoms,
(CH₂)ₙOR′, (CH₂)ₙCOOR′, (CH₂)ₙCONR′R″, (CH₂)ₙS(O)₂NR′R″,
(CH₂)ₙS(O)₂R⁸, or heteroaryl;
C₁-C₁₀ alkyl, C₃-C₈ cycloalkyl or C₅-C₈ cycloalkenyl, said alkyl, cycloalkyl or cycloalkenyl optionally containing one or more heteroatoms(s); unsubstituted or substituted with OH, CO₂R′ or CONR′R″;
COOR′; C(O)R′; SO₂NHR⁸ or SO₂R⁸;
or N, R and R⁷ taken together form a 4-7 member ring, optionally containing up to 2 heteroatoms selected from O, N and S, said heteroatom(s) being optionally substituted; said ring optionally substituted with lower alkyl, OH, benzyl, phenyl,
CO₂R′ or CONR′R″;
R⁸ is aryl, aralkyl, alkyl, heteroaryl or heteroaralkyl; optionally substituted;
R⁹ is C₁-C₆ alkyl or H; R′ and R″ are each independently H, C₁-C₁₂ alkyl, aryl, or alkyl or taken together form a 4-7 member ring;
n is 0-2; and wherein 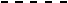
   is a bond or is absent.

Further provided is a compound having a formula 21, 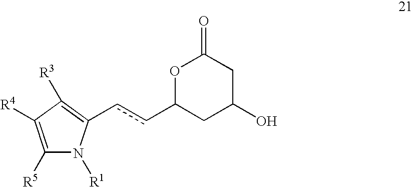
or a pharmaceutically acceptable salt, ester, amide, stereoisomer, racemic mixture or prodrug thereof, or a pharmaceutically acceptable salt of the prodrug, wherein
R¹ is lower alkyl, optionally substituted with a halogen;
R³ is benzyl; naphthyl; C₃-C₈ cycloalkyl or C₅-C₈ cycloalkenyl, optionally substituted with one or more heteroatom(s); phenyl or phenyl substituted with one or more groups selected from fluorine, chlorine, bromine, hydroxyl or alkyl of from one to seven carbon atoms; pyridinyl or pyridinyl substituted with fluorine, chlorine, bromine, hydroxyl or alkyl of from one to seven carbon atoms;
R⁴ is H; aryl, aralkyl, heteroaryl or heteroaralkyl; optionally substituted with one or more groups selected from fluorine, chlorine, bromine, hydroxyl or alkyl of from one to seven carbon atoms;
C₁-C₈ alkyl or C₃-C₈ cycloalkyl; optionally substituted; aralkenyl; carbamoyl or substituted carbamoyl; carboxyl or substituted carboxyl;
R⁵ is H, I, phenyl, COOR′, R⁶R⁷NC(O)— or SO₂NR⁶R⁷;
R⁶ and R⁷ are each independently H; aryl, aralkyl, heteroaryl or heteroaralkyl; optionally substituted with halo, alkyl of from one to seven carbon atoms,
(CH₂)ₙOR′, (CH₂)ₙCOOR′, (CH₂)ₙCONR′R″, (CH₂)ₙS(O)₂NR′R″,
(CH₂)ₙS(O)₂R⁸, or heteroaryl;
C₁-C₁₀ alkyl, C₃-C₈ cycloalkyl or C₅-C₈ cycloalkenyl, said alkyl, cycloalkyl or cycloalkenyl optionally containing one or more heteroatoms(s); unsubstituted or substituted with OH, CO₂R′ or CONR′R″;
COOR′; C(O)R′; SO₂NHR⁸ or SO₂R⁸,
or N, R⁶ and R⁷ taken together form a 4-7 member ring, optionally containing up to 2 heteroatoms selected from O, N and S, said heteroatom(s) being optionally substituted; said ring optionally substituted with lower alkyl, OH, benzyl, phenyl,
CO₂R′ or CONR′R″;
R⁸ is aryl, aralkyl, alkyl, heteroaryl or heteroaralkyl; optionally substituted;
R′ and R″ are each independently H, C₁-C₁₂ alkyl, aryl, or alkyl or taken together form a 4-7 member ring;
n is 0-2; and wherein 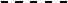
   is a bond or is absent.

Further provided is a process for making a compound of claim 43 having a formula, 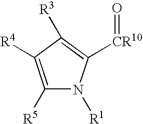
wherein R¹, R³, R⁴, R⁵ and R⁹ are as defined in claim 43 comprising the following steps:
1). Reacting a compound having a formula 5, 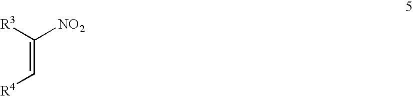
wherein R³ and R⁴ are as defined in claim 39, in a solvent, with ethyl isocyanoacetate to form a compound 6, 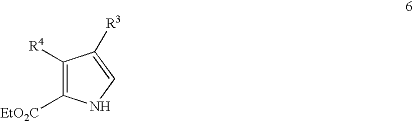
wherein R³ and R⁴ are as defined above and Et is ethyl;
2.) Alkylating the compound 6 to form a compound 7, 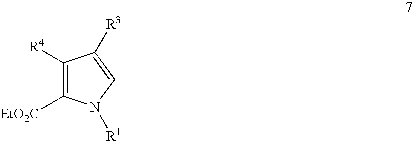
wherein R¹, R³, R⁴ and Et are as defined above; and
3.) Formylating the compound 7 to form the compound.

Further provided is a compound having a formula, 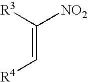
wherein R³ is benzyl; naphthyl; C₃-C₈ cycloalkyl or C₅-C₈ cycloalkenyl, optionally substituted with one or more heteroatom(s); phenyl or phenyl substituted with one or more groups selected from fluorine, chlorine, bromine, hydroxyl or alkyl of from one to seven carbon atoms; pyridinyl or pyridinyl substituted with fluorine, chlorine, bromine, hydroxyl or alkyl of from one to seven carbon atoms; and R⁴ is H; aryl, aralkyl, heteroaryl or heteroaralkyl; optionally substituted with one or more groups selected from fluorine, chlorine, bromine, hydroxyl or alkyl of from one to seven carbon atoms.

Still further provided is a compound selected from the group consisting of:
(4R,6R)-(6-{2-[3,4-Bis-(4-fluoro-phenyl)-1-isopropyl-1H-pyrrol-2-yl]-ethyl}-2,2-dimethyl-[1,3]dioxan-4-yl)-acetic acid;
6-{[5-(6-Carboxy-3,5-dihydroxy-hexyl)-4-(4-fluoro-phenyl)-1-isopropyl-3-phenyl-1H-pyrrole-2-carbonyl]-amino}-nicotinic acid;
7-[5-(Acetylamino-methyl)-3-(4-fluoro-phenyl)-1-isopropyl-4-phenyl-1H-pyrrol-2-yl]-3,5-dihydroxy-heptanoic acid; and pharmaceutically acceptable salts, esters and amides thereof.

Further provided is a compound having superior efficacy as an HMG-CoA reductase inhibitor as well as a high selectivity profile (cholesterol inhibition in hepatic vs. L6 muscle cells).

The following definitions are used, unless otherwise described. Halo is fluoro, chloro, bromo or iodo. Alkyl, alkoxy, alkenyl, alkynyl, etc. denote both straight and branched groups.

The term “alkyl” as used herein refers to a straight or branched hydrocarbon of from 1 to 11 carbon atoms and includes, for example, methyl, ethyl, n-propyl, isopropyl, n-butyl, sec-butyl, isobutyl, tert-butyl, n-pentyl, n-hexyl, and the like. The alkyl group can also be substituted with one or more of the substituents selected from lower alkoxy, lower thioalkoxy, —O(CH₂)₀₋₂CF₃, halogen, nitro, cyano, ═O, ═S, —OH, —SH, —CF₃, —CO₂H, —CO₂C₁-C₆ alkyl, —NH₂, —NHC₁-C₆ alkyl, —CONR′R″, or —N(C₁-C₆alkyl)₂ where R′ and R″ are independently alkyl, akenyl, alkynyl, aryl, or joined together to form a 4 to 7 member ring. Useful alkyl groups have from 1 to 6 carbon atoms (C₁-C₆ alkyl).

The term “lower alkyl” as used herein refers to a subset of alkyl which means a straight or branched hydrocarbon radical having from 1 to 6 carbon atoms and includes, for example, methyl, ethyl, n-propyl, isopropyl; n-butyl, sec-butyl, isobutyl, tert-butyl, n-pentyl, n-hexyl, and the like. Optionally, lower alkyl is referred to as “C₁-C₆ alkyl.”

The term “haloalkyl” as used herein refers to a lower alkyl radical, as defined above, bearing at least one halogen substituent, for example, chloromethyl, fluoroethyl, trifluoromethyl, or 1,1,1-trifluoroethyl and the like. Haloalkyl can also include perfluoroalkyl wherein all hydrogens of a loweralkyl group are replaced with fluorine atoms.

The term “alkenyl” means a straight or branched unsaturated hydrocarbon radical from 2 to 12 carbon atoms and includes, for example, ethenyl, 1-propenyl, 2-propenyl, 1-butenyl, 2-butenyl, 1-pentenyl, 2-pentenyl, 3-methyl-3-butenyl, 1-hexenyl, 2-hexenyl, 3-hexenyl, 3-heptenyl, 1-octenyl, 1-nonenyl, 1-decenyl, 1-undecenyl, 1-dodecenyl, and the like.

The term “alkynyl” means a straight or branched hydrocarbon radical of 2 to 12 carbon atoms having at least one triple bond and includes, for example, 3-propynyl, 1-butynyl, 3-butynyl, 1-pentynyl, 3-pentynyl, 3-methyl-3-butynyl, 1-hexynyl, 3-hexynyl, 3-hexynyl, 3-heptynyl, 1-octynyl, 1-nonynyl, 1-decynyl, 1-undecynyl, 1-dodecynyl, and the like.

The term “alkylene” as used herein refers to a divalent group derived from a straight or branched chain saturated hydrocarbon having from 1 to 10 carbon atoms by the removal of two hydrogen atoms, for example methylene, 1,2-ethylene, 1,1-ethylene, 1,3-propylene, 2,2-dimethylpropylene, and the like. The alkylene groups of this invention can be optionally substituted with one or more of the substituents selected from lower alkyl, lower alkoxy, lower thioalkoxy, —O(CH₂)₀₋₂CF₃, halogen, nitro, cyano, ═O, ═S, —OH, —SH, —CF₃, —CO₂H, —CO₂C₁-C₆ alkyl, —NH₂, —NHC₁-C₆ alkyl, —CONR′R″, or —N(C₁-C₆alkyl)₂ where R′ and R″ are independently alkyl, akenyl, alkynyl, aryl, or joined together to form a 4 to 7 member ring. Useful alkylene groups have from 1 to 6 carbon atoms (C₁-C₆ alkylene).

The term “heteroatom” as used herein represents oxygen, nitrogen, or sulfur (O, N, or S) as well as sulfoxyl or sulfonyl (SO or SO₂) unless otherwise indicated.

The term “hydrocarbon chain” as used herein refers to a straight hydrocarbon of from 2 to 6 carbon atoms. The hydrocarbon chain is optionally substituted with one or more substituents selected from lower alkyl, lower alkoxy, lower thioalkoxy, —O(CH₂)₀₋₂CF₃, halogen, nitro, cyano, ═O, ═S, —OH, —SH, —CF₃, —CO₂H, —CO₂C₁-C₆ alkyl, —NH₂, —NHC₁-C₆ alkyl, —CONR′R″, or —N(C₁-C₆alkyl)₂ where R′ and R″ are independently alkyl, alkenyl, alkynyl, aryl, or joined together to form a 4 to 7 member ring.

The term “hydrocarbon-heteroatom chain” as used herein refers to a hydrocarbon chain wherein one or more carbon atoms are replaced with a heteroatom. The hydrocarbon-heteroatom chain is optionally substituted with one or more substituents selected from lower alkyl, lower alkoxy, lower thioalkoxy, —O(CH₂)₀₋₂CF₃, halogen, nitro, cyano, ═O, ═S, —OH, —SH, —CF₃, —CO₂H, —CO₂C₁-C₆ alkyl, —NH₂, —NH(C₁-C₆ alkyl), —CONR′R″, or —N(C₁-C₆alkyl)₂ where R′ and R″ are independently alkyl, alkenyl, alkynyl, aryl, or joined together to form a 4 to 7 member ring.

The term “heteroalkylene” as used herein, refers to an alkylene radical as defined above that includes one or more heteroatoms such as oxygen, sulfur, or nitrogen (with valence completed by hydrogen or oxygen) in the carbon chain or terminating the carbon chain.

The terms “lower alkoxy” and “lower thioalkoxy” as used herein refers to O-alkyl or S-alkyl of from 1 to 6 carbon atoms as defined above for “lower alkyl.”

The term “aryl” as used herein refers to an aromatic ring which is unsubstituted or optionally substituted by 1 to 4 substituents selected from lower alkyl, lower alkoxy, lower thioalkoxy, —O(CH₂)PCF₃, halogen, nitro, cyano —OH, —SH, —CF₃, —CO₂H, —CO₂C₁-C₆ alkyl, —NH₂, —NHC₁-C₆ alkyl, —SO₂alkyl, —SO₂NH₂, —CONR′R″, or —N(C₁-C₆alkyl)₂ where R′ and R″ are independently alkyl, alkenyl, alkynyl, aryl, or joined together to form a 4 to 7 member ring. Examples include, but are not limited to phenyl, biphenyl, naphthyl, 2-chlorophenyl, 3-chlorophenyl, 4-chlorophenyl, 2-methylphenyl, 3-methylphenyl, 4-methylphenyl, 2-methoxyphenyl, 3-methoxyphenyl, 4-methoxyphenyl, 2-chloro-3-methylphenyl, 2-chloro-4-methylphenyl, 2-chloro-5-methylphenyl, 3-chloro-2-methylphenyl, 3-chloro-4-methylphenyl, 4-chloro-2-methylphenyl, 4-chloro-3-methylphenyl, 5-chloro-2-methylphenyl, 2,3-dichlorophenyl, 2,5-dichlorophenyl, 3,4-dichlorophenyl, 2,3-dimethylphenyl, 3,4-dimethylphenyl, or the like. Further, the term “aryl” means a cyclic or polycyclic aromatic ring having from 5 to 12 carbon atoms, and being unsubstituted or substituted with up to 4 of the substituent groups recited above for alkyl, alkenyl, and alkynyl.

The term aralkyl as used herein means aryl, as defined above, attached to an alkyl group.

The term “heteroaryl” means an aromatic ring containing one or more heteroatom. The heteroaryl is optionally substituted with one or more groups enumerated for aryl. Examples of heteroaryl include, but are not limited to thienyl, furanyl, pyrrolyl, pyridyl, pyrimidyl, imidazoyl, pyrazinyl, oxazolyl, thiazolyl, benzothienyl, benzofuranyl, indolyl, quinolinyl, isoquinolinyl, and quinazolinyl, and the like. Further, the term “heteroaryl” means an aromatic mono-, bi-, or polycyclic ring incorporating one or more (i.e. 1-4) heteroatoms selected from N, O, and S, which mono-, bi-, or polycyclic ring is optionally substituted with —OH, —O(alkyl), SH, S(alkyl), amine, halogen, acid, ester, amide, amidine, alkyl ketone, aldehyde, nitrile, fluoroalkyl, nitro, sulphone, sulfoxide or C₁₋₆ alkyl. Examples further include 1-, 2-, 4-, or 5-imidazolyl, 1-, 3-, 4-, or 5-pyrazolyl, 2-, 4-, or 5-thiazolyl, 3-, 4-, or 5-isothiazolyl, 2-, 4-, or 5-oxazolyl, 3-, 4-, or 5-isoxazolyl, 1,3-, or 5-triazolyl, 1-, 2-, or 3-tetrazolyl, 2-pyrazinyl, 2-, 4-, or 5-pyrimidinyl. Examples of suitable bicyclic heteroaryl compounds include, but are not limited to indolizinyl, isoindolyl, benzofuranyl, benzothienyl, benzoxazolyl, benzimidazolyl, quinolinyl, isoquinolinyl, quinazolinyl, 1-, 2-, 3-, 4-, 5-, 6-, or 7-indolyl, 1-, 2-, 3-, 5-, 6-, 7-, or 8-indolizinyl, 1-, 2-, 3-, 4-, 5-, 6-, or 7-isoindolyl, 2-, 3-, 4-, 5-, 6-, or 7-benzothienyl, 2-, 4-, 5-, 6-, or 7-benzoxazolyl, 1-, 2-, 4-, 5-, 6-, or 7-benzimidazolyl, 2-, 3-, 4-, 5-, 6-, 7-, or 8-quinolinyl, and 1-, 3-, 4-, 5-, 6-, 7-, or 8-isoquinolinyl.

The term heteroaralkyl, as used herein, means heteroaryl, as defined above, attached to an alkyl group.

The term “heterocycle” means a saturated mono- or polycyclic (i.e. bicyclic) ring incorporating one or more (i.e. 1-4) heteroatoms selected from N, O, and S. It is understood that a heterocycle is optionally substituted with —OH, —O(alkyl), SH, S(alkyl), amine, halogen, acid, ester, amide, amidine, alkyl ketone, aldehyde, nitrile, fluoroalkyl, nitro, sulphone, sulfoxide or C1-6 alkyl. Examples of suitable monocyclic heterocycles include, but are not limited to piperidinyl, pyrrolidinyl, piperazinyl, azetidinyl, aziridinyl, morpholinyl, thietanyl, oxetaryl.

The term “cycloalkyl” means a saturated hydrocarbon ring. Further, the term “cycloalkyl” means a hydrocarbon ring containing from 3 to 12 carbon atoms, for example, cyclopropyl, cyclobutyl, cyclopentyl, cyclohexyl, cycloheptyl, cycloctyl, decalinyl, norpinanyl, and adamantyl. The cycloalkyl ring may be unsubstituted or substituted by 1 to 3 substituents selected from alkyl, alkoxy, thioalkoxy, hydroxy, thiol, nitro, halogen, amino, alkyl and dialkylamino, formyl, carboxyl, CN, —NH—CO—R——CO—NHR—, —CO₂R—, —COR—, aryl, or heteroaryl, wherein alkyl, aryl, and heteroaryl are as defined herein. Examples of substituted cycloalkyl groups include fluorocyclopropyl, 2-iodocyclobutyl, 2,3-dimethylcyclopentyl, 2,2-dimethoxycyclohexyl, and 3-phenylcyclopentyl.

The term “cycloalkenyl” means a cycloalkyl group having one or more carbon-carbon double bond. Example includes cyclobutene, cyclopentene, cyclohexene, cycloheptene, cyclobutadiene, cyclopentadiene, and the like.

The term “isomer” means “stereoisomer” and “geometric isomer” as defined below.

The term “stereoisomer” means compounds that possess one or more chiral centers and each center may exist in the R or S configuration. Stereoisomers includes all diastereomeric, enantiomeric and epimeric forms as well as racemates and mixtures thereof.

The term “geometric isomer” means compounds that may exist in cis, trans syn, anti, entgegen (E), and, zusammen (Z) forms as well as mixtures thereof.

The symbol “=” means a double bond.

The symbol “∩” means a bond to a group wherein a 4 to 8 membered ring is formed. Typically this symbol will appear in pairs.

When a bond to a substituent is shown to cross the bond connecting 2 atoms in a ring, then such substituent may be bonded to any atom in the ring, provided the atom will accept the substituent without violating its valency. When there appears to be several atoms of the substituent that may bond to the ring atom, then it is the first atom of the listed substituent that is attached to the ring.

When a bond from a substituent is shown to cross the bond connecting 2 atoms in a ring of the substituent, then such substituent may be bonded from any atom in the ring which is available.

When a bond is represented by a line such as 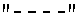
this is meant to represent that the bond may be absent or present provided that the resultant compound is stable and of satisfactory valency. If an asymetric carbon is created by such a bond, a particular stereochemistry is not to be implied.

As used herein, the following terms have the meanings given: RT means room temperature. MP means melting point. MS means mass spectroscopy. TLC means thin layer chromatography. [S]at. means saturated. [C]onc. means concentrated. TBIA means tert-Butylisopropylidene amine. DCM means dichloromethane, which is used interchangeably with methylene chloride. NBS means N-Bromosuccinimide. “h” means hour. “v/v” means volume ratio or “volume per volume”. R_{f} means retention factor. Tf₂O means “triflic anhydride” or C(F)₃S(O)₂OS(O)₂C(F)₃ or (CF₃SO₂)₂O. Ac₂O means acetic anhydride. “[T]rifluorotol.” means trifluorotoluene. “DMF” means dimethylformamide. “DCE” means dichloroethane. “Bu” means butyl. “Me” means methyl. “Et” means ethyl. “DBU” means 1,8-Diazabicyclo-[5.4.0]undec-7-ene. “TBDMS” means tert-Butyldimethylsilyl. “DMSO” means dimethyl sulfoxide.

The term “patient” means all mammals including humans. Examples of patients include humans, cows, dogs, cats, goats, sheep, pigs, and rabbits.

A “therapeutically effective amount” is an amount of a compound of the present invention that when administered to a patient ameliorates a symptom of hyperlipidemia, hypercholesterolemia, hypertriglyceridemia or atheroscelerois.

The term “a pharmaceutically acceptable salt, ester, amide, or prodrug” as used herein refers to those carboxylate salts, amino acid addition salts, esters, amides, and prodrugs of the compounds of the present invention which are, within the scope of sound medical judgment, suitable for use in contact with the tissues of patients without undue toxicity, irritation, allergic response, and the like, commensurate with a reasonable benefit/risk ratio, and effective for their intended use, as well as the zwitterionic forms, where possible, of the compounds of the invention. The term “a pharmaceutically acceptable salt” refers to the relatively non-toxic, inorganic and organic acid or base addition salts of compounds of the present invention. These salts can be prepared in situ during the final isolation and purification of the compounds or by separately reacting the purified compound in its free form with a suitable organic or inorganic acid or base and isolating the salt thus formed. Representative salts include the hydrobromide, hydrochloride, sulfate, bisulfate, nitrate, acetate, oxalate, valerate, oleate, palmitate, stearate, laurate, borate, benzoate, lactate, phosphate, tosylate, citrate, maleate, fumarate, succinate, tartrate, naphthylate mesylate, glucoheptonate, lactobionate, and laurylsulphonate salts, and the like. Pharmaceutically acceptable salts also include cations based on the alkali and alkaline earth metals, such as sodium, lithium, potassium, calcium, magnesium, and the like, as well as non-toxic ammonium, quaternary ammonium, and amine cations including, but not limited to ammonium, tetramethylammonium, tetraethylammonium, methylamine, dimethylamine, trimethylamine, triethylamine, ethylamine, and the like. (See, for example, Berge S. M., et al., “Pharmaceutical Salts,” J. Pharm. Sci., 1977; 66: 1-19, which is incorporated herein by reference.) The free base form may be regenerated by contacting the salt form with a base. While the free base may differ from the salt form in terms of physical properties, such as solubility, the salts are equivalent to their respective free bases for the purposes of the present invention.

Examples of pharmaceutically acceptable, non-toxic esters of the compounds of this invention include C₁-C₆ alkyl esters wherein the alkyl group is a straight or branched chain. Acceptable esters also include C₅-C₇ cycloalkyl esters as well as arylalkyl esters such as, but not limited to benzyl. C₁-C₄ alkyl esters are preferred. Esters of the compounds of the present invention may be prepared according to conventional methods.

Examples of pharmaceutically acceptable, non-toxic amides of the compounds of this invention include amides derived from ammonia, primary C₁-C₆ alkyl amines and secondary C₁-C₆ dialkyl amines wherein the alkyl groups are straight or branched chain. In the case of secondary amines, the amine may also be in the form of a 5- or 6-membered heterocycle containing one nitrogen atom. Amides derived from ammonia, C₁-C₃ alkyl primary amines and C₁-C₂ dialkyl secondary amines are preferred. Amides of the compounds of the invention may be prepared according to conventional methods.

“Prodrugs” are intended to include any covalently bonded carrier which releases the active parent drug according to Formula I in vivo. Further, the term “prodrug” refers to compounds that are transformed in vivo to yield the parent compound of the above formulae, for example, by hydrolysis in blood. A thorough discussion is provided in T. Higuchi and V. Stella, “Pro-drugs as Novel Delivery Systems,” Vol. 14 of the A.C.S. Symposium Series, and in Bioreversible Carriers in Drug Design, ed. Edward B. Roche, American Pharmaceutical Association and Pergamon Press, 1987, both of which are hereby incorporated by reference. Examples of prodrugs include acetates, formates, benzoate derivatives of alcohols, and amines present in compounds of Formula I.

In some situations, compounds may exist as tautomers. All tautomers are included within Formula I and are provided by this invention.

Certain compounds of the present invention can exist in unsolvated form as well as solvated form including hydrated form. In general, the solvated form including hydrated form is equivalent to unsolvated form and is intended to be encompassed within the scope of the present invention.

Certain of the compounds of the present invention possess one or more chiral centers and each center may exist in the R or S configuration. The present invention includes all diastereomeric, enantiomeric, and epimeric forms as well as the appropriate mixtures thereof. Stereoisomers may be obtained, if desired, by methods known in the art as, for example, the separation of stereoisomers by chiral chromatographic columns and by chiral synthesis. Additionally, the compounds of the present invention may exist as geometric isomers. The present invention includes all cis, trans, syn, anti, entgegen (E), and zusammen (Z) isomers as well as the appropriate mixtures thereof.

The compounds of the present invention are suitable to be administered to a patient for the treatment, control, or prevention of, hypercholesteremia, hyperlipidemia, atherosclerosis and hypertriglyceridemia. The terms “treatment”, “treating”, “controlling”, “preventing” and the like, refers to reversing, alleviating, or inhibiting the progress of the disease or condition to which such term applies, or one or more symptoms of such disease or condition. As used herein, these terms also encompass, depending on the condition of the patient, preventing the onset of a disease or condition or of symptoms associated with a disease or condition, including reducing the severity of a disease or condition or symptoms associated therewith prior to affliction with said disease or condition. Such prevention or reduction prior to affliction refers to administration of the compound of the invention to a subject that is not at the time of administration afflicted with the disease or condition. “Preventing” also encompasses preventing the recurrence of a disease or condition or of symptoms associated therewith. Accordingly, the compounds of the present invention can be administered to a patient alone or as part of a composition that contains other components such as excipients, diluents, and carriers, all of which are well-known in the art. The compositions can be administered to humans and animals either orally, rectally, parenterally (intravenously, intramuscularly, or subcutaneously), intracisternally, intravaginally, intraperitoneally, intravesically, locally (powders, ointments, or drops), or as a buccal or nasal spray.

Compositions suitable for parenteral injection may comprise physiologically acceptable sterile aqueous or nonaqueous solutions, dispersions, suspensions or emulsions, and sterile powders for reconstitution into sterile injectable solutions or dispersions. Examples of suitable aqueous and nonaqueous carriers, diluents, solvents or vehicles include water, ethanol, polyols (propyleneglycol, polyethyleneglycol, glycerol, and the like), suitable mixtures thereof, vegetable oils (such as olive oil), and injectable organic esters such as ethyl oleate. Proper fluidity can be maintained, for example, by the use of a coating such as lecithin, by the maintenance of the required particle size in the case of dispersions and by the use of surfactants.

These compositions may also contain adjuvants such as preserving, wetting, emulsifying, and dispensing agents. Prevention of the action of microorganisms can be ensured by various antibacterial and antifungal agents, for example, parabens, chlorobutanol, phenol, sorbic acid, and the like. It may also be desirable to include isotonic agents, for example sugars, sodium chloride, and the like. Prolonged absorption of the injectable pharmaceutical form can be brought about by the use of agents delaying absorption, for example, aluminum monostearate and gelatin.

Solid dosage forms for oral administration include capsules, tablets, pills, powders, and granules. In such solid dosage forms, the active compound is admixed with at least one inert customary excipient (or carrier) such as sodium citrate or dicalcium phosphate or (a) fillers or extenders, as for example, starches, lactose, sucrose, glucose, mannitol, and silicic acid; (b) binders, as for example, carboxymethylcellulose, alignates, gelatin, polyvinylpyrrolidone, sucrose, and acacia; (c) humectants, as for example, glycerol; (d) disintegrating agents, as for example, agar-agar, calcium carbonate, potato or tapioca starch, alginic acid, certain complex silicates, and sodium carbonate; (e) solution retarders, as for example paraffin; (f) absorption accelerators, as for example, quaternary ammonium compounds; (g) wetting agents, as for example, cetyl alcohol and glycerol monostearate; (h) adsorbents, as for example, kaolin and bentonite; and (i) lubricants, as for example, talc, calcium stearate, magnesium stearate, solid polyethylene glycols, sodium lauryl sulfate, or mixtures thereof. In the case of capsules, tablets, and pills, the dosage forms may also comprise buffering agents.

Solid compositions of a similar type may also be employed as fillers in soft and hard-filled gelatin capsules using such excipients as lactose or milk sugar as well as high molecular weight polyethyleneglycols, and the like.

Solid dosage forms such as tablets, dragees, capsules, pills, and granules can be prepared with coatings and shells, such as enteric coatings and others well-known in the art. They may contain opacifying agents, and can also be of such composition that they release the active compound or compounds in a certain part of the intestinal tract in a delayed manner. Examples of embedding compositions which can be used are polymeric substances and waxes. The active compounds can also be in micro-encapsulated form, if appropriate, with one or more of the above-mentioned excipients.

Liquid dosage forms for oral administration include pharmaceutically acceptable emulsions, solutions, suspensions, syrups, and elixirs. In addition to the active compounds, the liquid dosage forms may contain inert diluents commonly used in the art, such as water or other solvents, solubilizing agents and emulsifiers, as for example, ethyl alcohol, isopropyl alcohol, ethyl carbonate, ethyl acetate, benzyl alcohol, benzyl benzoate, propyleneglycol, 1,3-butyleneglycol, dimethylformamide, oils, in particular, cottonseed oil, groundnut oil, corn germ oil, olive oil, castor oil and sesame oil, glycerol, tetrahydrofurfuryl alcohol, polyethyleneglycols and fatty acid esters of sorbitan or mixtures of these substances, and the like.

Besides such inert diluents, the composition can also include adjuvants, such as wetting agents, emulsifying and suspending agents, sweetening, flavoring, and perfuming agents.

Suspensions, in addition to the active compounds, may contain suspending agents, as for example, ethoxylated isostearyl alcohols, polyoxyethylene sorbitol and sorbitan esters, microcrystalline cellulose, aluminum metahydroxide, bentonite, agar-agar and tragacanth, or mixtures of these substances, and the like.

Compositions for rectal administrations are preferably suppositories which can be prepared by mixing the compounds of the present invention with suitable non-irritating excipients or carriers such as cocoa butter, polyethyleneglycol, or a suppository wax, which are solid at ordinary temperatures but liquid at body temperature and therefore, melt in the rectum or vaginal cavity and release the active component.

Dosage forms for topical administration of a compound of this invention include ointments, powders, sprays, and inhalants. The active component is admixed under sterile conditions with a physiologically acceptable carrier and any preservatives, buffers, or propellants as may be required. Ophthalmic formulations, eye ointments, powders, and solutions are also contemplated as being within the scope of this invention.

The compounds of the present invention can be administered to a patient at dosage levels in the range of about 0.1 to about 2,000 mg per day. For a normal human adult having a body weight of about 70 kilograms, a dosage in the range of about 0.01 to about 100 mg per kilogram of body weight per day is preferable. The specific dosage used, however, can vary. For example, the dosage can depend on a numbers of factors including the requirements of the patient, the severity of the condition being treated, and the pharmacological activity of the compound being used. The determination of optimum dosages for a particular patient is well-known to those skilled in the art.

Combination Aspect of the Invention

The compounds of this invention may be used, either alone or in combination with the other pharmaceutical agents described herein, in the treatment of the following diseases/conditions: dyslipidemia, hypercholesterolemia, hypertriglyceridemia, atherosclerosis, peripheral vascular disease, cardiovascular disorders, angina, ischemia, cardiac ischemia, stroke, myocardial infarction, reperfusion injury, angioplastic restenosis, hypertension, diabetes and vascular complications of diabetes, obesity, unstable angina pectoris, Alzheimer's Disease, BPH, osteoporosis, cerebrovascular disease, coronary artery disease, ventricular dysfunction, cardiac arrhythmia, pulmonary vascular disease, renal-vascular disease, renal disease, vascular hemostatic disease, autoimmune disorders, pulmonary disease, anti-oxidant disease, sexual dysfunction, cognitive dysfunction, cancer, organ transplant rejection, psoriasis, endometriosis, and macular degeneration.

The compounds of this invention may also be used in conjunction with other pharmaceutical agents (e.g., HDL-cholesterol raising agents, triglyceride lowering agents) for the treatment of the disease/conditions described herein. A combination aspect of this invention includes a pharmaceutical composition comprising a compound of this invention or its pharmaceutically acceptable salt and at least one other compound. For example, the compounds of this invention may be used in combination with cholesterol absorption inhibitors, MTP/Apo B secretion inhibitors, or other cholesterol modulating agents such as fibrates, niacin, ion-exchange resins, antioxidants, ACAT inhibitors, PPAR-activators, CETP inhibitors or bile acid sequestrants. In combination therapy treatment, both the compounds of this invention and the other drug therapies are administered to mammals by conventional methods. The following discussion more specifically describes the various combination aspects of this invention.

Any cholesterol absorption inhibitor can be used in a combination aspect of this invention. The term cholesterol absorption inhibition refers to the ability of a compound to prevent cholesterol contained within the lumen of the intestine from entering into the intestinal cells and/or passing from within the intestinal cells into the blood stream. Such cholesterol absorption inhibition activity is readily determined by those skilled in the art according to standard assays (e.g., J. Lipid Res. (1993) 34: 377-395). Cholesterol absorption inhibitors are known to those skilled in the art and are described, for example, in PCT WO 94/00480. An example of a recently approved cholesterol absorption inhibitor is ZETIA™.

Any cholesterol ester transfer protein (“CETP”) inhibitor may be used in a combination aspect of this invention. The term CETP inhibitor refers to compounds that inhibit the transfer of cholesteryl ester and triglyceride between lipoprotein particles, including high density lipoproteins (HDL), low density lipoproteins (LDL), very low density lipoproteins (VLDL), and chylomicrons. The net result of CETP activity is a lowering of HDL cholesterol and an increase in LDL cholesterol, such net effect therefore being pro-atherogenic. Thus, the effect of a CETP inhibitor on lipoprotein profile is believed to be anti-atherogenic. Such inhibition is readily determined by those skilled in the art by determining the amount of agent required to alter plasma lipid levels, for example HDL cholesterol levels, LDL cholesterol levels, VLDL cholesterol levels or triglycerides, in the plasma of certain mammals, (e.g., Crook et al. Arteriosclerosis 10, 625, 1990; U.S. Pat. No. 6,140,343). A variety of these compounds are described and referenced below, however other CETP inhibitors will be known to those skilled in the art. For example, U.S. Pat. Nos. 6,197,786, 6,723,752 and 6,723,753 (the disclosures of each of which is incorporated herein by reference) disclose cholesteryl ester transfer protein inhibitors, pharmaceutical compositions containing such inhibitors and the use of such inhibitors to elevate certain plasma lipid levels, including high density lipoprotein-cholesterol and to lower certain other plasma lipid levels, such as LDL-cholesterol and triglycerides and accordingly to treat diseases which are exacerbated by low levels of HDL cholesterol and/or high levels of LDL-cholesterol and triglycerides, such as atherosclerosis and cardiovascualar diseases in some mammals, including humans. Examples of useful CETP inhibitors include the following compounds: [2R, 4S]4-[(3,5-bis-trifluoromethyl-benzyl)-methoxycarbonyl-amino]-2-ethyl-6-trifluoromethyl-3,4-dihydroxycarbonyl-amino]-2-ethyl-6-trifluoromethyl-3,4-dihydro-2H-quinoline-1-carboxylic acid ethyl ester, which is also known as Torcetrapib™, and 3-{[3-(4-Chloro-3-ethyl-phenoxy)-phenyl]-[3-(1,1,2,2-tetrafluoro-ethoxy)-benzyl]-amino}-1,1,1-trifluoro-propan-2-ol. Many of the CETP inhibitors of this invention are poorly soluble and a dosage form that increases solubility facilitates the administration of such compounds. One such dosage form is a dosage form comprising (1) a solid amorphous dispersion comprising a cholesteryl ester transfer protein (CETP) inhibitor and an acidic concentration-enhancing polymer; and (2) an acid-sensitive HMG-CoA reductase inhibitor. This dosage form is more fully described in U.S. Ser. No. 10/739,567 and entitled “Dosage Forms Comprising a CETP Inhibitor and an HMG-CoA Reductase Inhibitor”, the specification of which is incorporated herein by reference.

Any compound that activates or otherwise interacts with a human peroxisome proliferator activated receptor (“PPAR”) may be used in a combination aspect of this invention.

Three mammalian peroxisome proliferator-activated receptors have been isolated and termed PPAR-alpha, PPAR-gamma, and PPAR-beta (also known as NUC1 or PPAR-delta). These PPARs regulate expression of target genes by binding to DNA sequence elements, termed PPAR response elements. These elements have been identified in the enhancers of a number of genes encoding proteins that regulate lipid metabolism suggesting that PPARs play a pivotal role in the adipogenic signaling cascade and lipid homeostasis. PPAR-gamma receptors are associated with regulation of insulin sensitivity and blood glucose levels. PPAR-α activators are associated with lowering plasma triglycerides and LDL cholesterol. PPAR-β activators have been reported to both increase HDL-C levels and to decrease LDL-C levels. Thus, activation of PPAR-P alone, or in combination with the simultaneous activation of PPAR-αand/or PPAR-gamma may be desirable in formulating a treatment for dyslipidemia in which HDL is increased and LDL lowered. PPAR-activation is readily determined by those skilled in the art by the standard assays (e.g. U.S. 2003/0225158 and U.S. 2004/0157885). A variety of these compounds are described and referenced below, however other PPAR-activator compounds will be known to those skilled in the art. The following patents and published patent applications, the disclosure of each of which is incorporated herein by reference, provides a sampling. U.S. 2003/0225158 discloses compounds that alter PPAR activity and methods of using them as therapeutic agents for treating or preventing dyslipidemia, hypercholesterolemia, obesity, hyperglycemia, atherosclerosis and hypertriglyceridemia. U.S. Pat. No. 6,710,063 discloses selective activators of PPAR delta. U.S. 2003/0171377 discloses certain PPAR-activator compounds that are useful as anti-diabetic agents. U.S. 2004/0157885 relates to PPAR agonists, in particular, certain PPARα agonists, pharmaceutical compositions containing such agonists and the use of such agonists to treat atherosclerosis, hypercholesterolemia, hypertriglyceridemia, diabetes, obesity, osteoporosis and Syndrome X or metabolic syndrome.

Examples of useful PPAR-activator compounds include the following compounds: [5-Methoxy-2-methly-4-(4′-trifluoromethly-biphenyl-4ylmethylsulfanyl)-phenoxy]-acetic acid; [5-Methoxy-2-methyl-4-(3′-trifloromethly-biphenyl-4-ylmethylsulfanyl)-phenoxy]-acetic acid;
[4-(4′Fluoro-biphenyl-4-ylmethylsulfanyl)-5-methoxy-2methyl-phenoxy]-acetic acid;
{5-Methoxy-2methyl-4-[4-(4-trifluoromethyl-benzyloxy)-benzylsulfanyl]-phenoxy}-acetic acid; {{5-Methoxy-2-methyl-4-[4-(5-trifluoromethyl-pryidin-2-yl)-benzylsulfanyl]-phenoxy}-acetic acid;
(4-{4-[2-(3-Fluoro-phenyl)-vinyl]-benzylsulfanyl-5-methoxy-2-methyl-phenoxy)-acetic acid; [5-Methoxy-2-methyl-4-(3-methyl-4′-trifluoromethyl-biphenyl-4-ylmethylsulfanyl)-phenoxy]-acetic acid; [5-Methoxy-2-methyl-4-(4′-trifluoromethyl-biphenyl-3-ylmethylsulfanyl)-phenoxy]-acetic acid;
{5-Methoxy-2-methyl-4-[2-(4-trifluoromethyl-benzyloxy)-benzylsulfanyl]-phenoxy acetic acid; 3-{5-[2-(-5-Methyl-2 phenyl-oxazol-4-yl-ethoxy]-indol-1-yl}-propionic acid; 3-{4[2-(5-methyl-2-phenyl-1,3-oxazol-4-yl)ethoxy-1H-indazol-1yl}propanoic acid; 2-Methyl-2-{3-[({2-(5-methyl-2-phenyl-1,3-oxazol-4-yl)ethoxy]carbonyl} amino)methyl]phenoxy}propionic acid; 1-{3′-[2-5-Methyl-2-phenyl-1,3-oxazol-4-y]-1,1′-biphenyl-3-yl} oxy)cyclobutanecarboxylic acid;
3-[3-(1-Carboxy-1-methyl-ethoxy)-phenyl]-piperidine-1-carboxylic acid 3-trifluoromethyl-benzyl ester;
2-{2-methyl-4-[({4-methyl-2-[4-(trifluoromethyl)phenyl]-1,3-thiazol-5-yl} methyl) sulfanyl]phenoxy}acetic acid;
2-{2-methyl-4-[({4-methyl-2-[4-(trifluoromethyl)phenyl]-1,3-oxazol-5-yl} methyl)sulfanyl]phenoxy} acetic acid;
methyl 2-{4-[({4-methyl-2-[4-(trifluoromethyl)phenyl]-1,3-thiazol-5-yl} methyl)sulfanyl]phenoxy} acetate;
2-{4-[({4-methyl-2-[4-(trifluoromethyl)phenyl]-1,3-thiazol-5-yl} methyl)sulfanyl]phenoxy} acetic acid;
(E)-3-[2-methyl-4-({4-methyl-2-[4-(trifluoromethyl)phenyl]-1,3-thiazol-5-yl) methoxy)phenyl]-2-propenoic acid;
2-{3-chloro-4-[({4-methyl-2-[4-(trifluoromethyl)phenyl]-1,3-thiazol-5-yl} methyl) sulfanyl]phenyl} acetic acid;
2-{2-methyl-4-[({4-methyl-2-[3-fluoro-4-(trifluoromethyl)phenyl]-1,3-thiazol-5-yl}methyl)sulfanyl]phenoxy}acetic acid; and pharmaceutically acceptable salts thereof.

Any MTP/Apo B secretion (microsomal triglyceride transfer protein and/or apolipoprotein B secretion) inhibitor can be used in the combination aspect of the present invention. The term MTP/Apo B secretion inhibitor refers to compounds, which inhibit the secretion of triglycerides, cholesteryl ester and phospholipids. Such inhibition is readily determined by those skilled in the art according to standard assays (e.g., Wetterau, J. R. 1992; Science 258: 999). A variety of these compounds are known to those skilled in the art, including imputapride (Bayer) and additional compounds such as those disclosed in WO 96/40640 and WO 98/23593.

Any squalene synthetase inhibitor can be used as the second compound of the present invention. The term squalene synthetase inhibitor refers to compounds, which inhibit the condensation of 2 molecules of farnesylpyrophosphate to form squalene, catalyzed by the enzyme squalene synthetase. Such inhibition is readily determined by those skilled in the art according to standard assays (e.g., Meth. Enzymol. 1969; 15: 393-454 and Meth. Enzymol. 1985; 110: 359-373 and references contained therein). A variety of these compounds are known to those skilled in the art, for example, U.S. Pat. No. 5,026,554 discloses fermentation products of the microorganism MF5465 (ATCC 74011) including zaragozic acid. A summary of other squalene synthetase inhibitors has been compiled (see, e.g., Curr. Op. Ther. Patents (1993) 861-4).

Any squalene epoxidase inhibitor can be used in the combination aspect of the present invention. The term squalene epoxidase inhibitor refers to compounds that inhibit the bioconversion of squalene and molecular oxygen into squalene-2,3-epoxide, catalyzed by the enzyme squalene epoxidase. Such inhibition is readily determined by those skilled in the art according to standard assays (e.g., Biochim. Biophys. Acta 1984; 794: 466-471). A variety of these compounds are known to those skilled in the art, for example, U.S. Pat. Nos. 5,011,859 and 5,064,864 disclose certain fluoro analogs of squalene. EP publication 395,768 A discloses certain substituted allylamine derivatives. PCT publication WO 9312069 A discloses certain amino alcohol derivatives. U.S. Pat. No. 5,051,534 discloses certain cyclopropyloxy-squalene derivatives.

Any squalene cyclase inhibitor can be used in the combination aspect of the present invention. The term squalene cyclase inhibitor refers to compounds that inhibit the bioconversion of squalene-2,3-epoxide to lanosterol, catalyzed by the enzyme squalene cyclase. Such inhibition is readily determined by those skilled in the art according to standard assays (e.g., FEBS Lett. 1989; 244: 347-350). Squalene cyclase inhibitors are known to those skilled in the art. For example, PCT publication WO9410150 and French patent publication 2697250 disclose squalene cyclase inhibitors.

Any combined squalene epoxidase/squalene cyclase inhibitor can be used in the combination aspect of the present invention. The term combined squalene epoxidase/squalene cyclase inhibitor refers to compounds that inhibit the bioconversion of squalene to lanosterol via a squalene-2,3-epoxide intermediate. In some assays, it is not possible to distinguish between squalene epoxidase inhibitors and squalene cyclase inhibitors. However, these assays are recognized by those skilled in the art. Thus, inhibition by combined squalene epoxidase/squalene cyclase inhibitors is readily determined by those skilled in art according to the aforementioned standard assays for squalene cyclase or squalene epoxidase inhibitors. A variety of squalene epoxidase/squalene cyclase inhibitors are known to those skilled in the art. U.S. Pat. Nos. 5,084,461 and 5,278,171 disclose certain azadecalin derivatives. EP publication 468,434 discloses certain piperidyl ether and thio-ether derivatives such as 2-(1-piperidyl)pentyl isopentyl sulfoxide and 2-(1-piperidyl)ethyl ethyl sulfide. PCT publication WO 9401404 discloses certain acyl-piperidines such as 1-(1-oxopentyl-5-phenylthio)-4-(2-hydroxy-1-methyl)-ethyl)piperidine. U.S. Pat. No. 5,102,915 discloses certain cyclopropyloxy-squalene derivatives.

Any ACAT inhibitor can serve in the combination therapy aspect of the present invention. The term ACAT inhibitor refers to compounds that inhibit the intracellular esterification of dietary cholesterol by the enzyme acyl CoA: cholesterol acyltransferase. Such inhibition may be determined readily by one of skill in the art according to standard assays, such as the method of Heider et al. described in Journal of Lipid Research., 24: 1127 (1983). A variety of these compounds are known to those skilled in the art, for example, U.S. Pat. No. 5,510,379 discloses certain carboxysulfonates, while WO 96/26948 and WO 96/10559 both disclose urea derivatives having ACAT inhibitory activity. Examples of ACAT inhibitors include compounds such as Avasimibe (Pfizer), CS-505 (Sankyo) and Eflucimibe (Eli Lilly and Pierre Fabre).

A lipase inhibitor can serve in the combination therapy aspect of the present invention. A lipase inhibitor is a compound that inhibits the metabolic cleavage of dietary triglycerides into free fatty acids and monoglycerides. Under normal physiological conditions, lipolysis occurs via a two-step process that involves acylation of an activated serine moiety of the lipase enzyme. This leads to the production of a fatty acid-lipase hemiacetal intermediate, which is then cleaved to release a diglyceride. Following further deacylation, the lipase-fatty acid intermediate is cleaved, resulting in free lipase, a monoglyceride and a fatty acid. The resultant free fatty acids and monoglycerides are incorporated into bile acid-phospholipid micelles, which are subsequently absorbed at the level of the brush border of the small intestine. The micelles eventually enter the peripheral circulation as chylomicrons. Such lipase inhibition activity is readily determined by those skilled in the art according to standard assays (e.g., Methods Enzymol. 286: 190-231).

Pancreatic lipase mediates the metabolic cleavage of fatty acids from triglycerides at the 1- and 3-carbon positions. The primary site of the metabolism of ingested fats is in the duodenum and proximal jejunum by pancreatic lipase, which is usually secreted in vast excess of the amounts necessary for the breakdown of fats in the upper small intestine. Because pancreatic lipase is the primary enzyme required for the absorption of dietary triglycerides, inhibitors have utility in the treatment of obesity and the other related conditions. Such pancreatic lipase inhibition activity is readily determined by those skilled in the art according to standard assays (e.g., Methods Enzymol. 286: 190-231).

Gastric lipase is an immunologically distinct lipase that is responsible for approximately 10 to 40% of the digestion of dietary fats. Gastric lipase is secreted in response to mechanical stimulation, ingestion of food, the presence of a fatty meal or by sympathetic agents. Gastric lipolysis of ingested fats is of physiological importance in the provision of fatty acids needed to trigger pancreatic lipase activity in the intestine and is also of importance for fat absorption in a variety of physiological and pathological conditions associated with pancreatic insufficiency. See, for example, C. K. Abrams, et al., Gastroenterology, 92,125 (1987). Such gastric lipase inhibition activity is readily determined by those skilled in the art according to standard assays (e.g., Methods Enzymol. 286: 190-231).

A variety of gastric and/or pancreatic lipase inhibitors are known to one of ordinary skill in the art. Preferred lipase inhibitors are those inhibitors that are selected from the group consisting of lipstatin, tetrahydrolipstatin (orlistat), valilactone, esterastin, ebelactone A, and ebelactone B. The compound tetrahydrolipstatin is especially preferred. The lipase inhibitor, N-3-trifluoromethylphenyl-N′-3-chloro-4′-trifluoromethylphenylurea, and the various urea derivatives related thereto, are disclosed in U.S. Pat. No. 4,405,644. The lipase inhibitor, esteracin, is disclosed in U.S. Pat. Nos. 4,189,438 and 4,242,453. The lipase inhibitor, cyclo-O,O′-[(1,6-hexanediyl)-bis-(iminoc-arbonyl)]dioxime, and the various bis(iminocarbonyl) dioximes related thereto may be prepared as described in Petersen et al., Liebig's Annalen, 562, 205-229 (1949).

A variety of pancreatic lipase inhibitors are described herein below. The pancreatic lipase inhibitors lipstatin, (2S,3S,5S,7Z,10Z)-5-[(S)-2-formamido-4-methyl-valeryloxy]-2-hexyl-3-hydro-xy-7,10-hexadecanoic acid lactone, and tetrahydrolipstatin (orlistat), (2S,3S,5S)-5-[(S)-2-formamido-4-methyl-valeryloxy]-2-hexyl-3-hydroxy-hexa-decanoic 1,3 acid lactone, and the variously substituted N-formylleucine derivatives and stereoisomers thereof, are disclosed in U.S. Pat. No. 4,598,089. For example, tetrahydrolipstatin is prepared as described in, e.g., U.S. Pat. Nos. 5,274,143; 5,420,305; 5,540,917; and 5,643,874. The pancreatic lipase inhibitor, FL-386, 1-[4-(2-methylpropyl)cyclohexyl]-2-[-(phenylsulfonyl)oxy]-ethanone, and the variously substituted sulfonate derivatives related thereto, are disclosed in U.S. Pat. No. 4,452,813. The pancreatic lipase inhibitor, WAY-121898, 4-phenoxyphenyl-4-methylpiperidin-1-yl-carboxylate, and the various carbamate esters and pharmaceutically acceptable salts related thereto, are disclosed in U.S. Pat. Nos. 5,512,565; 5,391,571 and 5,602,151. The pancreatic lipase inhibitor, valilactone, and a process for the preparation thereof by the microbial cultivation of Actinomycetes strain MG147-CF2, are disclosed in Kitahara, et al., J. Antibiotics, 40 (11), 1647-1650 (1987). The pancreatic lipase inhibitors, ebelactone A and ebelactone B, and a process for the preparation thereof by the microbial cultivation of Actinomycetes strain MG7-G1, are disclosed in Umezawa, et al., J. Antibiotics, 33, 1594-1596 (1980). The use of ebelactones A and B in the suppression of monoglyceride formation is disclosed in Japanese Kokai 08-143457, published Jun. 4, 1996.

Other compounds that are marketed for hyperlipidemia, including hypercholesterolemia and which are intended to help prevent or treat atherosclerosis include bile acid sequestrants, such as Welchol®, Colestid®, LoCholest®, Questran® and fibric acid derivatives, such as Atromid®, Lopid® and Tricor®.

Compunds of the present invention can be used with anti-diabetic compounds. Diabetes can be treated by administering to a patient having diabetes (especially Type II), insulin resistance, impaired glucose tolerance, or the like, or any of the diabetic complications such as neuropathy, nephropathy, retinopathy or cataracts, a therapeutically effective amount of a Formula I compound in combination with other agents (e.g., insulin) that can be used to treat diabetes. This includes the classes of anti-diabetic agents (and specific agents) described herein.

Any glycogen phosphorylase inhibitor can be used in combination with a Formula I compound of the present invention. The term glycogen phosphorylase inhibitor refers to compounds that inhibit the bioconversion of glycogen to glucose-1-phosphate which is catalyzed by the enzyme glycogen phosphorylase. Such glycogen phosphorylase inhibition activity is readily determined by those skilled in the art according to standard assays (e.g., J. Med. Chem. 41 (1998) 2934-2938). A variety of glycogen phosphorylase inhibitors are known to those skilled in the art including those described in WO 96/39384 and WO 96/39385.

Any aldose reductase inhibitor can be used in combination with a Formula I compound of the present invention. The term aldose reductase inhibitor refers to compounds that inhibit the bioconversion of glucose to sorbitol, which is catalyzed by the enzyme aldose reductase. Aldose reductase inhibition is readily determined by those skilled in the art according to standard assays (e.g., J. Malone, Diabetes, 29: 861-864 (1980). “Red Cell Sorbitol, an Indicator of Diabetic Control”). A variety of aldose reductase inhibitors are known to those skilled in the art.

Any sorbitol dehydrogenase inhibitor can be used in combination with a Formula I compound of the present invention. The term sorbitol dehydrogenase inhibitor refers to compounds that inhibit the bioconversion of sorbitol to fructose which is catalyzed by the enzyme sorbitol dehydrogenase. Such sorbitol dehydrogenase inhibitor activity is readily determined by those skilled in the art according to standard assays (e.g., Analyt. Biochem (2000) 280: 329-331). A variety of sorbitol dehydrogenase inhibitors are known, for example, U.S. Pat. Nos. 5,728,704 and 5,866,578 disclose compounds and a method for treating or preventing diabetic complications by inhibiting the enzyme sorbitol dehydrogenase.

Any glucosidase inhibitor can be used in combination with a Formula I compound of the present invention. A glucosidase inhibitor inhibits the enzymatic hydrolysis of complex carbohydrates by glycoside hydrolases, for example amylase or maltase, into bioavailable simple sugars, for example, glucose. The rapid metabolic action of glucosidases, particularly following the intake of high levels of carbohydrates, results in a state of alimentary hyperglycemia which, in adipose or diabetic subjects, leads to enhanced secretion of insulin, increased fat synthesis and a reduction in fat degradation. Following such hyperglycemias, hypoglycemia frequently occurs, due to the augmented levels of insulin present. Additionally, it is known chyme remaining in the stomach promotes the production of gastric juice, which initiates or favors the development of gastritis or duodenal ulcers. Accordingly, glucosidase inhibitors are known to have utility in accelerating the passage of carbohydrates through the stomach and inhibiting the absorption of glucose from the intestine. Furthermore, the conversion of carbohydrates into lipids of the fatty tissue and the subsequent incorporation of alimentary fat into fatty tissue deposits is accordingly reduced or delayed, with the concomitant benefit of reducing or preventing the deleterious abnormalities resulting therefrom. Such glucosidase inhibition activity is readily determined by those skilled in the art according to standard assays (e.g., Biochemistry (1969) 8: 4214).

A generally preferred glucosidase inhibitor includes an amylase inhibitor. An amylase inhibitor is a glucosidase inhibitor that inhibits the enzymatic degradation of starch or glycogen into maltose. Such amylase inhibition activity is readily determined by those skilled in the art according to standard assays (e.g., Methods Enzymol. (1955) 1: 149). The inhibition of such enzymatic degradation is beneficial in reducing amounts of bioavailable sugars, including glucose and maltose, and the concomitant deleterious conditions resulting therefrom.

A variety of glucosidase inhibitors are known to one of ordinary skill in the art and examples are provided below. Preferred glucosidase inhibitors are those inhibitors that are selected from the group consisting of acarbose, adiposine, voglibose, miglitol, emiglitate, camiglibose, tendamistate, trestatin, pradimicin-Q and salbostatin. The glucosidase inhibitor, acarbose, and the various amino sugar derivatives related thereto are disclosed in U.S. Pat. Nos. 4,062,950 and 4,174,439 respectively. The glucosidase inhibitor, adiposine, is disclosed in U.S. Pat. No. 4,254,256. The glucosidase inhibitor, voglibose, 3,4-dideoxy-4-[[2-hydroxy-1-(hydroxymethyl)ethyl]amino]-2-C-(hydroxymethy-1)-D-epi-inositol, and the various N-substituted pseudo-aminosugars related thereto, are disclosed in U.S. Pat. No. 4,701,559. The glucosidase inhibitor, miglitol, (2R,3R,4R,5S)-1-(2-hydroxyethyl)-2-(hydr-oxymethyl)-3,4,5-piperidinetriol, and the various 3,4,5-trihydroxypiperidines related thereto, are disclosed in U.S. Pat. No. 4,639,436. The glucosidase inhibitor, emiglitate, ethyl p-[2-[(2R,3R,4R,5S)-3,4,5-trihydroxy-2-(hydroxymethyl)piperidino]ethoxy]-benzoate, the various derivatives related thereto and pharmaceutically acceptable acid addition salts thereof, are disclosed in U.S. Pat. No. 5,192,772. The glucosidase inhibitor, MDL-25637, 2,6-dideoxy-7-O-.beta.-D-glucopyrano-syl-2,6-imino-D-glycero-L-gluco-heptitol, the various homodisaccharides related thereto and the pharmaceutically acceptable acid addition salts thereof, are disclosed in U.S. Pat. No. 4,634,765. The glucosidase inhibitor, camiglibose, methyl 6-deoxy-6-[(2R,3R,4R,5S)-3,4,5-trihydroxy-2-(hydroxymethyl)piperidino]-.alpha.-D-glucopyranoside sesquihydrate, the deoxy-nojirimycin derivatives related thereto, the various pharmaceutically acceptable salts thereof and synthetic methods for the preparation thereof, are disclosed in U.S. Pat. Nos. 5,157,116 and 5,504,078. The glycosidase inhibitor, salbostatin and the various pseudosaccharides related thereto, are disclosed in U.S. Pat. No. 5,091,524.

A variety of amylase inhibitors are known to one of ordinary skill in the art. The amylase inhibitor, tendamistat and the various cyclic peptides related thereto, are disclosed in U.S. Pat. No. 4,451,455. The amylase inhibitor AI-3688 and the various cyclic polypeptides related thereto are disclosed in U.S. Pat. No. 4,623,714. The amylase inhibitor, trestatin, consisting of a mixture of trestatin A, trestatin B and trestatin C and the various trehalose-containing aminosugars related thereto are disclosed in U.S. Pat. No. 4,273,765.

Additional anti-diabetic compounds, which can be used in combination with a Formula I compound of the present invention, includes, for example, the following: biguanides (e.g., metformin), insulin secretagogues (e.g., sulfonylureas and glinides), glitazones, non-glitazone PPAR.gamma. agonists, PPAR.beta. agonists, inhibitors of DPP-IV, inhibitors of PDE5, inhibitors of GSK-3, glucagon antagonists, inhibitors of f-1,6-BPase (Metabasis/Sankyo), GLP-1/analogs (AC 2993, also known as exendin-4), insulin and insulin mimetics (Merck natural products). Other examples would include PKC-.beta. inhibitors and AGE breakers.

Compounds of the present invention can be used in combination with anti-obesity agents. Any anti-obesity agent can be used in such combinations and examples are provided herein. Such anti-obesity activity is readily determined by those skilled in the art according to standard assays known in the art. Suitable anti-obesity agents include phenylpropanolamine, ephedrine, pseudoephedrine, phentermine, .beta.sub.3 adrenergic receptor agonists, apolipoprotein-B secretion/microsomal triglyceride transfer protein (apo-B/MTP) inhibitors, MCR-4 agonists, cholecystokinin-A (CCK-A) agonists, monoamine reuptake inhibitors (e.g., sibutramine), sympathomimetic agents, serotoninergic agents, cannabinoid receptor antagonists (e.g., rimonabant (SR-141,716A)), dopamine agonists (e.g., bromocriptine), melanocyte-stimulating hormone receptor analogs, 5HT2c agonists, melanin concentrating hormone antagonists, leptin (the OB protein), leptin analogs, leptin receptor agonists, galanin antagonists, lipase inhibitors (e.g., tetrahydrolipstatin, i.e. orlistat), bombesin agonists, anorectic agents (e.g., a bombesin agonist), Neuropeptide-Y antagonists, thyroxine, thyromimetic agents, dehydroepiandrosterones or analogs thereof, glucocorticoid receptor agonists or antagonists, orexin receptor antagonists, urocortin binding protein antagonists, glucagon-like peptide-1 receptor agonists, ciliary neurotrophic factors (e.g., Axokine.TM.), human agouti-related proteins (AGRP), ghrelin receptor antagonists, histamine 3 receptor antagonists or inverse agonists, neuromedin U receptor agonists, and the like.

Any thyromimetic can be used in combination with compounds of the present invention. Such thyromimetic activity is readily determined by those skilled in the art according to standard assays (e.g., Atherosclerosis (1996) 126: 53-63). A variety of thyromimetic agents are known to those skilled in the art, for example those disclosed in U.S. Pat. Nos. 4,766,121; 4,826,876; 4,910,305; 5,061,798; 5,284,971; 5,401,772; 5,654,468; and 5,569,674. Other antiobesity agents include sibutramine which can be prepared as described in U.S. Pat. No. 4,929,629 and bromocriptine which can be prepared as described in U.S. Pat. Nos. 3,752,814 and 3,752,888.

Osteoporosis is a systemic skeletal disease, characterized by low bone mass and deterioration of bone tissue, with a consequent increase in bone fragility and susceptibility to fracture. In the U.S., the condition affects more than 25 million people and causes more than 1.3 million fractures each year, including 500,000 spine, 250,000 hip and 240,000 wrist fractures annually. Hip fractures are the most serious consequence of osteoporosis, with 5-20% of patients dying within one year, and over 50% of survivors being incapacitated. The elderly are at greatest risk of osteoporosis, and the problem is therefore predicted to increase significantly with the aging of the population. Worldwide fracture incidence is forecasted to increase three-fold over the next 60 years, and one study has estimated that there will be 4.5 million hip fractures worldwide in 2050. Women are at greater risk of osteoporosis than men. Women experience a sharp acceleration of bone loss during the five years following menopause. Other factors that increase the risk include smoking, alcohol abuse, a sedentary lifestyle and low calcium intake.

Those skilled in the art will recognize that anti-resorptive agents (for example progestins, polyphosphonates, bisphosphonate(s), estrogen agonists/antagonists, estrogen, estrogen/progestin combinations, Premarin.RTM., estrone, estriol or 17.alpha.- or 17.beta.-ethynyl estradiol) may be used in conjunction with the compounds of Formula I of the present invention. Exemplary progestins are available from commercial sources and include: algestone acetophenide, altrenogest, amadinone acetate, anagestone acetate, chlormadinone acetate, cingestol, clogestone acetate, clomegestone acetate, delmadinone acetate, desogestrel, dimethisterone, dydrogesterone, ethynerone, ethynodiol diacetate, etonogestrel, flurogestone acetate, gestaclone, gestodene, gestonorone caproate, gestrinone, haloprogesterone, hydroxyprogesterone caproate, levonorgestrel, lynestrenol, medrogestone, medroxyprogesterone acetate, melengestrol acetate, methynodiol diacetate, norethindrone, norethindrone acetate, norethynodrel, norgestimate, norgestomet, norgestrel, oxogestone phenpropionate, progesterone, quingestanol acetate, quingestrone, and tigestol. Preferred progestins are medroxyprogestrone, norethindrone and norethynodrel.

Exemplary bone resorption inhibiting polyphosphonates include polyphosphonates of the type disclosed in U.S. Pat. No. 3,683,080, the disclosure of which is incorporated herein by reference. Preferred polyphosphonates are geminal diphosphonates (also referred to as bis-phosphonates). Tiludronate disodium is an especially preferred polyphosphonate. Ibandronic acid is an especially preferred polyphosphonate. Alendronate and resindronate are especially preferred polyphosphonates. Zoledronic acid is an especially preferred polyphosphonate. Other preferred polyphosphonates are 6-amino-1-hydroxy-hexylidene-bisphosphonic acid and 1-hydroxy-3(methylpentylamino)-propylidene-bisphosphonic acid. The polyphosphonates may be administered in the form of the acid, or of a soluble alkali metal salt or alkaline earth metal salt. Hydrolyzable esters of the polyphosphonates are likewise included. Specific examples include ethane-1-hydroxy 1,1-diphosphonic acid, methane diphosphonic acid, pentane-1-hydroxy-1,1-diphosphonic acid, methane dichloro diphosphonic acid, methane hydroxy diphosphonic acid, ethane-1-amino-1,1-diphosphonic acid, ethane-2-amino-1,1-diphosphonic acid, propane-3-amino-1-hydroxy-1,1-diphosphonic acid, propane-N,N-dimethyl-3-amino-1-hydroxy-1,1-diphosphonic acid, propane-3,3-dimethyl-3-amino-1-hydroxy-1,1-diphosphonic acid, phenyl amino methane diphosphonic acid, N,N-dimethylamino methane diphosphonic acid, N(2-hydroxyethyl) amino methane diphosphonic acid, butane-4-amino-1-hydroxy-1,1-diphosphonic acid, pentane-5-amino-1-hydroxy-1,1-diphosphonic acid, hexane-6-amino-1-hydroxy-1,1-diphosphonic acid and pharmaceutically acceptable esters and salts thereof.

In particular, the compounds of this invention may be combined with a mammalian estrogen agonist/antagonist. Any estrogen agonist/antagonist may be used as the second compound of this invention. The term estrogen agonist/antagonist refers to compounds which bind with the estrogen receptor, inhibit bone turnover and/or prevent bone loss. In particular, estrogen agonists are herein defined as chemical compounds capable of binding to the estrogen receptor sites in mammalian tissue, and mimicking the actions of estrogen in one or more tissue. Estrogen antagonists are herein defined as chemical compounds capable of binding to the estrogen receptor sites in mammalian tissue, and blocking the actions of estrogen in one or more tissues. Such activities are readily determined by those skilled in the art of standard assays including estrogen receptor binding assays, standard bone histomorphometric and densitometer methods, and Eriksen E. F. et al., Bone Histomorphometry, Raven Press, New York, 1994, pages 1-74; Grier S. J. et. al., The Use of Dual-Energy X-Ray Absorptiometry In Animals, Inv. Radiol., 1996, 31(1): 50-62; Wahner H. W. and Fogelman I., The Evaluation of Osteoporosis: Dual Energy X-Ray Absorptiometry in Clinical Practice., Martin Dunitz Ltd., London 1994, pages 1-296). A variety of these compounds are described and referenced below. Another preferred estrogen agonist/antagonist is 3-(4-(1,2-diphenyl-but-1-enyl)-phenyl)-acrylic acid, which is disclosed in Willson et al., Endocrinology, 1997, 138, 3901-3911. Another preferred estrogen agonist/antagonist is tamoxifen: (ethanamine, 2-(-4-(1,2-diphenyl-1-butenyl)phenoxy)-N,N-dimethyl, (Z)-2-, 2-hydroxy-1,2,3-propanetricarboxylate (1:1)) and related compounds which are disclosed in U.S. Pat. No. 4,536,516, the disclosure of which is incorporated herein by reference. Another related compound is 4-hydroxy tamoxifen, which is disclosed in U.S. Pat. No. 4,623,660, the disclosure of which is incorporated herein by reference.

A preferred estrogen agonist/antagonist is raloxifene: (methanone, (6-hydroxy-2-(4-hydroxyphenyl)benzo[b]thien-3-yl)(4-(2-(1-piperidinyl)eth-oxy)phenyl)-hydrochloride) which is disclosed in U.S. Pat. No. 4,418,068, the disclosure of which is incorporated herein by reference.

Another preferred estrogen agonist/antagonist is toremifene: (ethanamine, 2-(4-(4-chloro-1,2-diphenyl-1-butenyl)phenoxy)-N,N-dimethyl-, (Z)-, 2-hydroxy-1,2,3-propanetricarboxylate (1:1) which is disclosed in U.S. Pat. No. 4,996,225, the disclosure of which is incorporated herein by reference. Another preferred estrogen agonist/antagonist is centchroman: 1-(2-((4-(-methoxy-2,2, dimethyl-3-phenyl-chroman-4-yl)-phenoxy)-ethyl)-p-pyrrolidine, which is disclosed in U.S. Pat. No. 3,822,287, the disclosure of which is incorporated herein by reference. Also preferred is levormeloxifene. Another preferred estrogen agonist/antagonist is idoxifene: (E)-1-(2-(4-(1-(4-iodo-phenyl)-2-phenyl-but-1-enyl)-phenoxy)-ethyl)-pyrro-lidinone, which is disclosed in U.S. Pat. No. 4,839,155, the disclosure of which is incorporated herein by reference.

Another preferred estrogen agonist/antagonist is 2-(4-methoxy-phenyl)-3-[4-(2-piperidin-1-yl-ethoxy)-phenoxy]-benzo[b]thio-phen-6-ol which is disclosed in U.S. Pat. No. 5,488,058, the disclosure of which is incorporated herein by reference.

Another preferred estrogen agonist/antagonist is 6-(4-hydroxy-phenyl)-5-(4-(2-piperidin-1-yl-ethoxy)-benzyl)-naphthalen-2-ol, which is disclosed in U.S. Pat. No. 5,484,795, the disclosure of which is incorporated herein by reference.

Another preferred estrogen agonist/antagonist is (4-(2-(2-aza-bicyclo [2.2.1]hept-2-yl)-ethoxy)-phenyl)-(6-hydroxy-2-(4-hyd-roxy-phenyl)-benzo[b]thiophen-3-yl)-methanone which is disclosed, along with methods of preparation, in PCT publication no. WO 95/10513 assigned to Pfizer Inc., the disclosure of which is incorporated herein by reference.

Other preferred estrogen agonist/antagonists include the compounds, TSE-424 (Wyeth-Ayerst Laboratories) and arazoxifene.

Other preferred estrogen agonist/antagonists include compounds as described in commonly assigned U.S. Pat. No. 5,552,412, the disclosure of which is incorporated herein by reference. Especially preferred compounds described therein are:
cis-6-(4-fluoro-phenyl)-5-(4-(2-piperidin-1-yl-ethoxy)-phenyl)-5,6,-7,8-tetrahydro-naphthalene-2-ol;
(−)-cis-6-phenyl-5-(4-(2-pyrrolidin-1-yl-ethoxy)-phenyl)-5,6,7,8-te-trahydro-naphthalene-2-ol (also known as lasofoxifene);
cis-6-phenyl-5-(4-(2-pyrrolidin-1-yl-ethoxy)-phenyl)-5,6,7,8-tetrah-ydro-naphthalene-2-ol;
cis-1-(6′-pyrrolodinoethoxy-3′-pyridyl)-2-phenyl-6-hydroxy-1,2,3,4-tetrahydronaphthalene;
1-(4′-pyrrolidinoethoxyphenyl)-2-(4″-fluorophenyl)-6-hydroxy-1,2,3,-4-tetrahydroisoquinoline;
is-6-(4-hydroxyphenyl)-5-(4-(2-piperidin-1-yl-ethoxy)-phenyl)-5,6,-7,8-tetrahydro-naphthalene-2-ol; and
1-(4′-pyrrolidinolethoxyphenyl)-2-phenyl-6-hydroxy-1,2,3,4-tetrahyd-roisoquinoline.

Other estrogen agonist/antagonists are described in U.S. Pat. No. 4,133,814 (the disclosure of which is incorporated herein by reference). U.S. Pat. No. 4,133,814 discloses derivatives of 2-phenyl-3-aroyl-benzoth-iophene and 2-phenyl-3-aroylbenzothiophene-1-oxide.

Other anti-osteoporosis agents, which can be used in combination with a Formula I compound of the present invention, include, for example, the following: parathyroid hormone (PTH) (a bone anabolic agent); parathyroid hormone (PTH) secretagogues (see, e.g., U.S. Pat. No. 6,132,774), particularly calcium receptor antagonists; calcitonin; and vitamin D and vitamin D analogs.

Any compound that is an antihypertensive agent may be used in a combination aspect of this invention. Such compounds include amlodipine and related dihydropyridine compounds, calcium channel blockers, angiotensin converting enzyme inhibitors (“ACE-Inhibitors”), angiotensin-II receptor antagonists, beta-adrenergic receptor blockers and alpha-adrenergic receptor blockers. Such antihypertensive activity is determined by those skilled in the art according to standard tests (e.g. blood pressure measurements).

Amlodipine and related dihydropyridine compounds are disclosed in U.S. Pat. No. 4,572,909, which is incorporated herein by reference, as potent anti-ischemic and antihypertensive agents. U.S. Pat. No. 4,879,303, which is incorporated herein by reference, discloses amlodipine benzenesulfonate salt (also termed amlodipine besylate). Amlodipine and amlodipine besylate are potent and long lasting calcium channel blockers. As such, amlodipine, amlodipine besylate and other pharmaceutically acceptable acid addition salts of amlodipine have utility as antihypertensive agents and as antiischemic agents. Amlodipine and its pharmaceutically acceptable acid addition salts are also disclosed in U.S. Pat. No. 5,155,120 as having utility in the treatment of congestive heart failure. Amlodipine besylate is currently sold as Norvasc®.

Calcium channel blockers which are within the scope of this invention include, but are not limited to: bepridil, which may be prepared as disclosed in U.S. Pat. No. 3,962,238 or U.S. Reissue No. 30,577; clentiazem, which may be prepared as disclosed in U.S. Pat. No. 4,567,175; diltiazem, which may be prepared as disclosed in U.S. Pat. No. 3,562, fendiline, which may be prepared as disclosed in U.S. Pat. No. 3,262,977; gallopamil, which may be prepared as disclosed in U.S. Pat. No. 3,261,859; mibefradil, which may be prepared as disclosed in U.S. Pat. No. 4,808,605; prenylamine, which may be prepared as disclosed in U.S. Pat. No. 3,152,173; semotiadil, which may be prepared as disclosed in U.S. Pat. No. 4,786,635; terodiline, which may be prepared as disclosed in U.S. Pat. No. 3,371,014; verapamil, which may be prepared as disclosed in U.S. Pat. No. 3,261,859; aranipine, which may be prepared as disclosed in U.S. Pat. No. 4,572,909; barnidipine, which may be prepared as disclosed in U.S. Pat. No. 4,220,649; benidipine, which may be prepared as disclosed in European Patent Application Publication No. 106,275; cilnidipine, which may be prepared as disclosed in U.S. Pat. No. 4,672,068; efonidipine, which may be prepared as disclosed in U.S. Pat. No. 4,885,284; elgodipine, which may be prepared as disclosed in U.S. Pat. No. 4,952,592; felodipine, which may be prepared as disclosed in U.S. Pat. No. 4,264,611; isradipine, which may be prepared as disclosed in U.S. Pat. No. 4,466,972; lacidipine, which may be prepared as disclosed in U.S. Pat. No. 4,801,599; lercanidipine, which may be prepared as disclosed in U.S. Pat. No. 4,705,797; manidipine, which may be prepared as disclosed in U.S. Pat. No. 4,892,875; nicardipine, which may be prepared as disclosed in U.S. Pat. No. 3,985,758; nifedipine, which may be prepared as disclosed in U.S. Pat. No. 3,485,847; nilvadipine, which may be prepared as disclosed in U.S. Pat. No. 4,338,322; nimodipine, which may be prepared as disclosed in U.S. Pat. No. 3,799,934; nisoldipine, which may be prepared as disclosed in U.S. Pat. No. 4,154,839; nitrendipine, which may be prepared as disclosed in U.S. Pat. No. 3,799,934; cinnarizine, which may be prepared as disclosed in U.S. Pat. No. 2,882,271; flunarizine, which may be prepared as disclosed in U.S. Pat. No. 3,773,939; lidoflazine, which may be prepared as disclosed in U.S. Pat. No. 3,267,104; lomerizine, which may be prepared as disclosed in U.S. Pat. No. 4,663,325; bencyclane, which may be prepared as disclosed in Hungarian Patent No. 151,865; etafenone, which may be prepared as disclosed in German Patent No. 1,265,758; and perhexiline, which may be prepared as disclosed in British Patent No. 1,025,578. The disclosures of all such U.S. Patents are incorporated herein by reference.

Angiotensin Converting Enzyme Inhibitors (ACE-Inhibitors) which are within the scope of this invention include, but are not limited to: alacepril, which may be prepared as disclosed in U.S. Pat. No. 4,248,883; benazepril, which may be prepared as disclosed in U.S. Pat. No. 4,410,520; captopril, which may be prepared as disclosed in U.S. Pat. Nos. 4,046,889 and 4,105,776; ceronapril, which may be prepared as disclosed in U.S. Pat. No. 4,452,790; delapril, which may be prepared as disclosed in U.S. Pat. No. 4,385,051; enalapril, which may be prepared as disclosed in U.S. Pat. No. 4,374,829; fosinopril, which may be prepared as disclosed in U.S. Pat. No. 4,337,201; imadapril, which may be prepared as disclosed in U.S. Pat. No. 4,508,727; lisinopril, which may be prepared as disclosed in U.S. Pat. No. 4,555,502; moveltopril, which may be prepared as disclosed in Belgian Patent No. 893,553; perindopril, which may be prepared as disclosed in U.S. Pat. No. 4,508,729; quinapril, which may be prepared as disclosed in U.S. Pat. No. 4,344,949; ramipril, which may be prepared as disclosed in U.S. Pat. No. 4,587,258; spirapril, which may be prepared as disclosed in U.S. Pat. No. 4,470,972; temocapril, which may be prepared as disclosed-in U.S. Pat. No. 4,699,905; and trandolapril, which may be prepared as disclosed in U.S. Pat. No. 4,933,361. The disclosures of all such U.S. patents are incorporated herein by reference.

Angiotensin-II receptor antagonists (A-II antagonists) which are within the scope of this invention include, but are not limited to: candesartan, which may be prepared as disclosed in U.S. Pat. No. 5,196,444; eprosartan, which may be prepared as disclosed in U.S. Pat. No. 5,185,351; irbesartan, which may be prepared as disclosed in U.S. Pat. No. 5,270,317; losartan, which may be prepared as disclosed in U.S. Pat. No. 5,138,069; and valsartan, which may be prepared as disclosed in U.S. Pat. No. 5,399,578. The disclosures of all such U.S. patents are incorporated herein by reference.

Beta-adrenergic receptor blockers (beta- or .beta.-blockers) which are within the scope of this invention include, but are not limited to: acebutolol, which may be prepared as disclosed in U.S. Pat. No. 3,857,952; alprenolol, which may be prepared as disclosed in Netherlands Patent Application No. 6,605,692; amosulalol, which may be prepared as disclosed in U.S. Pat. No. 4,217,305; arotinolol, which may be prepared as disclosed in U.S. Pat. No. 3,932,400; atenolol, which may be prepared as disclosed in U.S. Pat. No. 3,663,607 or 3,836,671; befunolol, which may be prepared as disclosed in U.S. Pat. No. 3,853,923; betaxolol, which may be prepared as disclosed in U.S. Pat. No. 4,252,984; The disclosures of all such U.S. patents are incorporated herein by reference.

Alpha-adrenergic receptor blockers (alpha- or alpha.-blockers) which are within the scope of this invention include, but are not limited to: amosulalol, which may be prepared as disclosed in U.S. Pat. No. 4,217,307; arotinolol, which may be prepared as disclosed in U.S. Pat. No. 3,932,400; dapiprazole, which may be prepared as disclosed in U.S. Pat. No. 4,252,721; doxazosin, which may be prepared as disclosed in U.S. Pat. No. 4,188,390; fenspiride, which may be prepared as disclosed in U.S. Pat. No. 3,399,192; indoramin, which may be prepared as disclosed in U.S. Pat. No. 3,527,761; labetolol, which may be prepared as disclosed above; naftopidil, which may be prepared as disclosed in U.S. Pat. No. 3,997,666; nicergoline, which may be prepared as disclosed in U.S. Pat. No. 3,228,943; prazosin, which may be prepared as disclosed in U.S. Pat. No. 3,511,836; tamsulosin, which may be prepared as disclosed in U.S. Pat. No. 4,703,063; tolazoline, which may be prepared as disclosed in U.S. Pat. No. 2,161,938; trimazosin, which may be prepared as disclosed in U.S. Pat. No. 3,669,968; and yohimbine, which may be isolated from natural sources according to methods well known to those skilled in the art. The disclosures of all such U.S. patents are incorporated herein by reference.

Any compound that is known to be useful in the treatment of Alzheimer's Disease may be used in a combination aspect of this invention. Such compounds include acetylcholine esterase inhibitors. Examples of known acetylcholine esterase inhibitors include donepezil (Aricept®), tacrine (Cognex®), rivastigmine (Exelon®) and galantamine (Reminyl). Aricept® is disclosed in the following U.S. patents, all of which are fully incorporated herein by reference: U.S. Pat. Nos. 4,895,841, 5,985,864, 6,140,321, 6,245,911 and 6,372,760. Exelon® is disclosed in U.S. Pat. Nos. 4,948,807 and 5,602,176 which are fully incorporated herein by reference.

Cognex® is disclosed in U.S. Pat. Nos. 4,631,286 and 4,816,456 (fully incorporated herein by reference). Remynil® is disclosed in U.S. Pat. Nos. 4,663,318 and 6,099,863 which are fully incorporated herein by reference.

### Preparation of Compounds of the Invention

The present invention contains compounds that can be synthesized in a number of ways familiar to one skilled in organic synthesis. The compounds outlined herein can be synthesized according to the methods described below, along with methods typically utilized by a synthetic chemist, and combinations or variations of those methods, which are generally known to one skilled in the art of synthetic chemistry. The synthetic route of compounds in the present invention is not limited to the methods outlined below. It is assumed that one skilled in the art will be able to use the schemes outlined below to synthesize compounds claimed in this invention. Individual compounds may require manipulation of the conditions in order to accommodate various functional groups. A variety of protecting groups generally known to one skilled in the art may be required. Purification, if necessary, can be accomplished on a silica gel column eluted with the appropriate organic solvent system. Also, reverse phase HPLC or recrystallization may be employed.

Scheme 1 shows the preparation of compounds of the invention wherein R¹ is isopropyl and R⁵ is phenyl-carbamoyl.

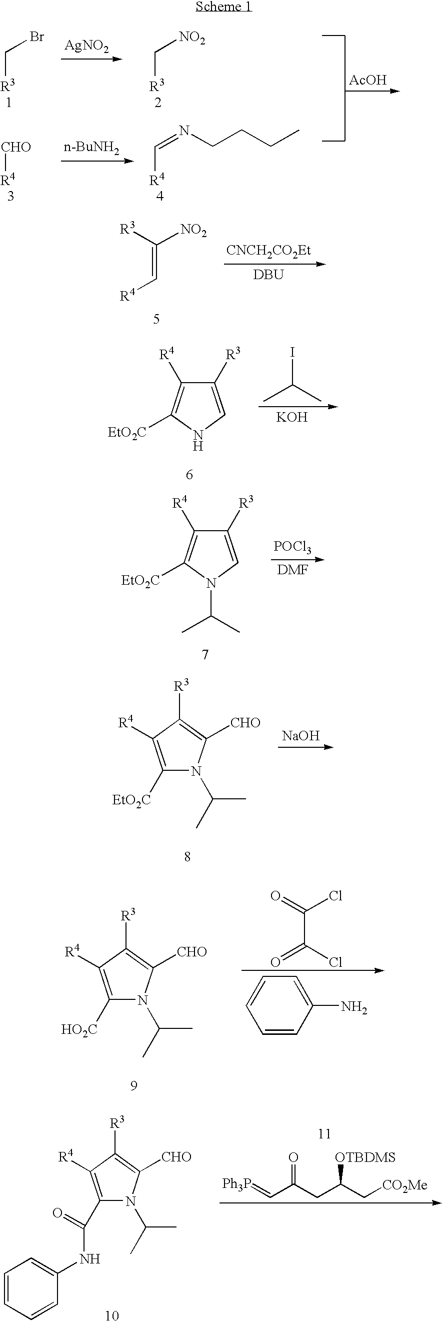

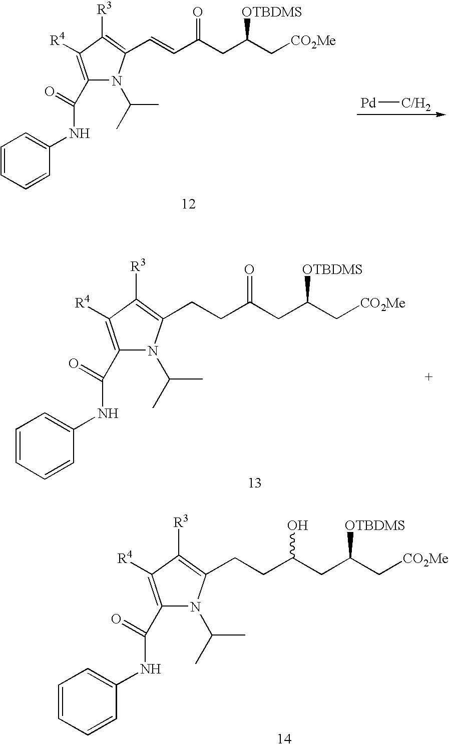

Scheme 1A shows a further example wherein R³ is para-fluorophenyl and R⁴ is phenyl.

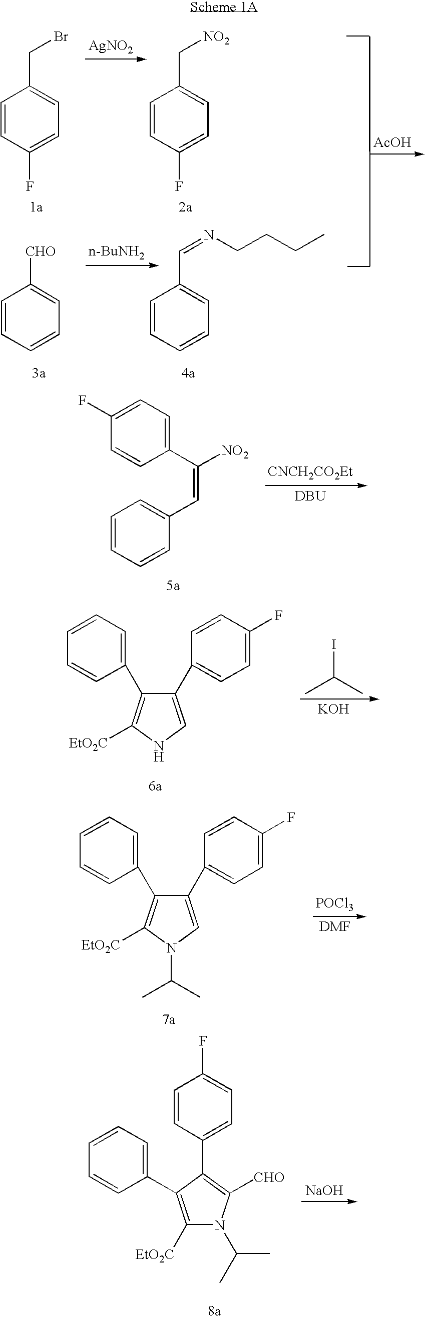

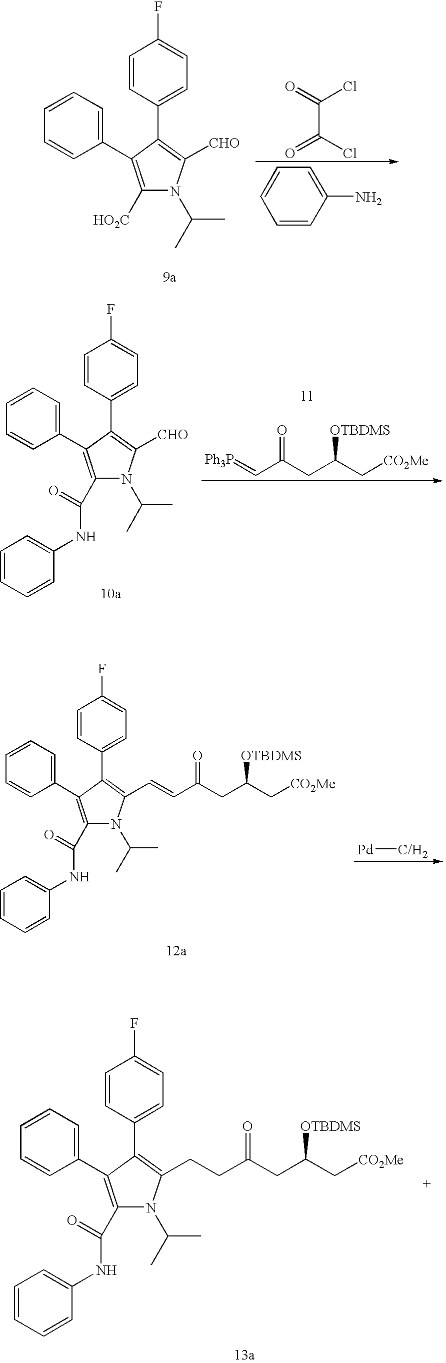

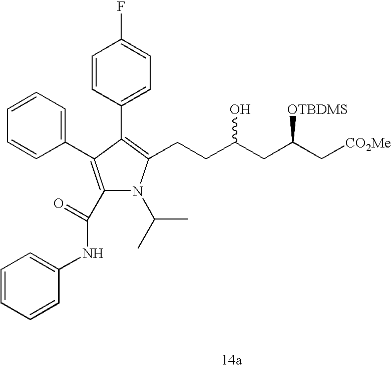

As shown in scheme 1A, compound 1a reacts with silver nitrite to give compound 2a following a procedure published by Kornblum et al (J. Am. Chem. Soc., 1955, 77, 6269). Nitrostilbene analog 5a can be made from the reaction of compound 2a with compound 4a as described by Dale Robertson (J. Org. Chem., 1960, 25, 47). Condensation reaction of compound 5a with ethyl isocyanoacetate gives compound 6a, which is alkylated to afford compound 7a. Formylation of compound 7a gives compound 8a. The aldehyde 10a can be obtained from compound 8a via standard hydrolysis and amide formation reactions. The Wittig reaction of compound 10a with the ylid 11 gives compound 12a, which can be converted to compound 13a via hydrogenation reaction. A diastereomeric mixture 14a is also isolated as aminor product from this reaction.

Scheme 2 shows the preparation of compounds of the invention wherein 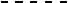
is absent, R¹ is isopropyl and R⁵ is phenylcarbamoyl.

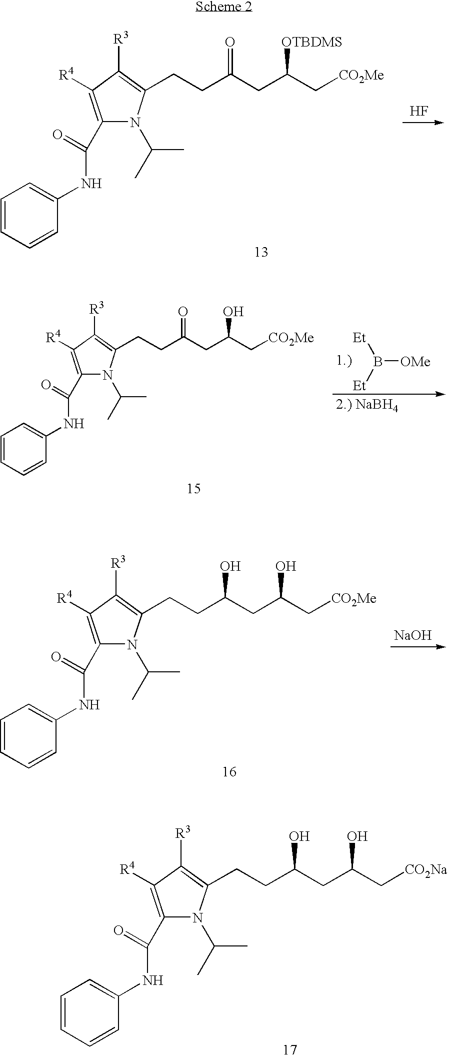

Scheme 2 shows the conversion of compound 13 to compound 17. Deprotection of compound 13 gives compound 15. Stereoselective reduction of compound 15 gives the diol 16. “Steroselective reduction” means treating the starting material with diethyl-methoxy-borane, then reducing with NaBH₄. Upon hydrolysis, compound 17 may be obtained. Alternatively, one could work up the reaction under acidic conditions to isolate the corresponding free acid. The transformations from compound 10 to compound 17 are carried out in a similar fashion as described in the patent EP 0521471B1 fully incorporated herein by reference. Alternatively, compound 16 can be obtained from compound 12 by a series of transformations shown in scheme 3.

Scheme 2A shows a further example wherein R³ is para-fluorophenyl and R⁴ is phenyl.

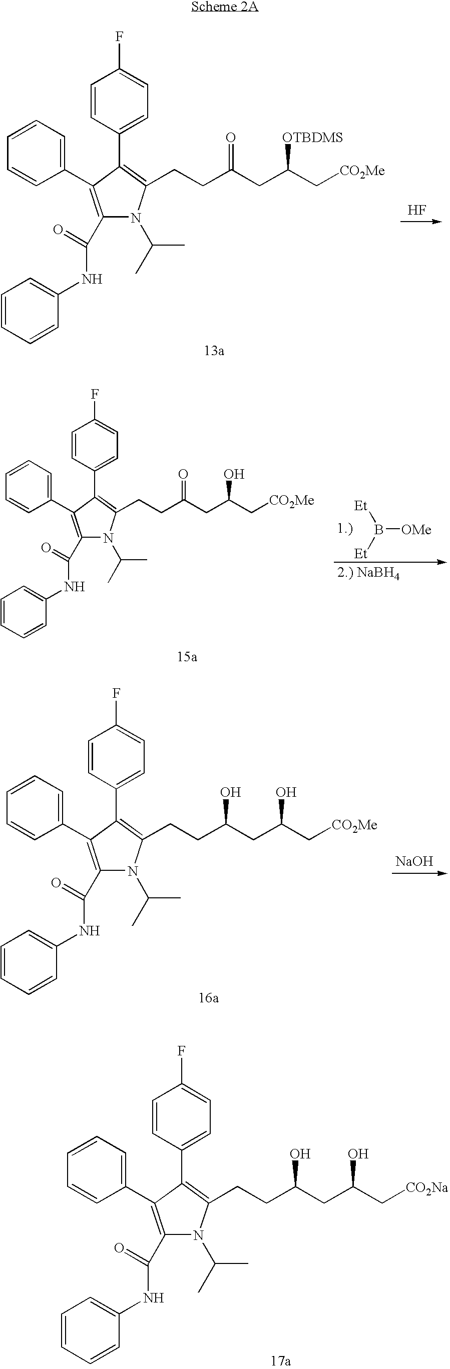

Scheme 3 shows the preparation of compounds of the invention wherein 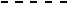
is a bond, R¹ is isopropyl and R⁵ is phenyl-carbamoyl.

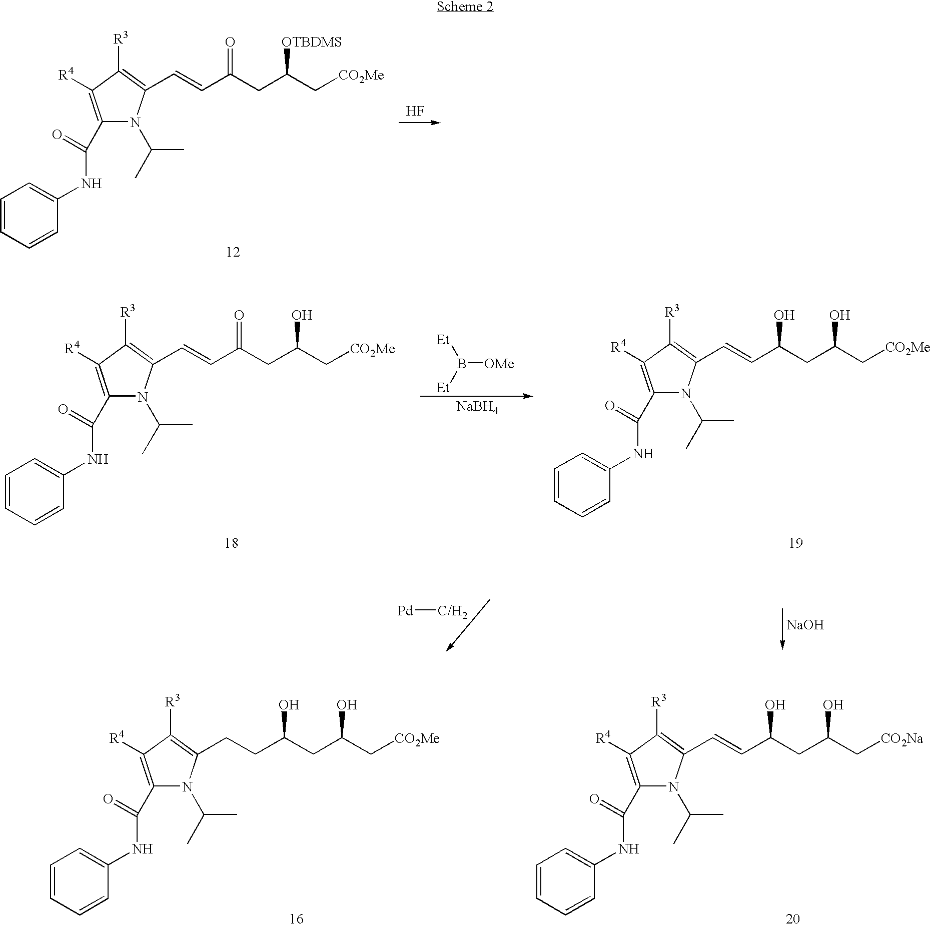

Scheme 3A shows a further example wherein R³ is para-fluorophenyl and R⁴ is phenyl.

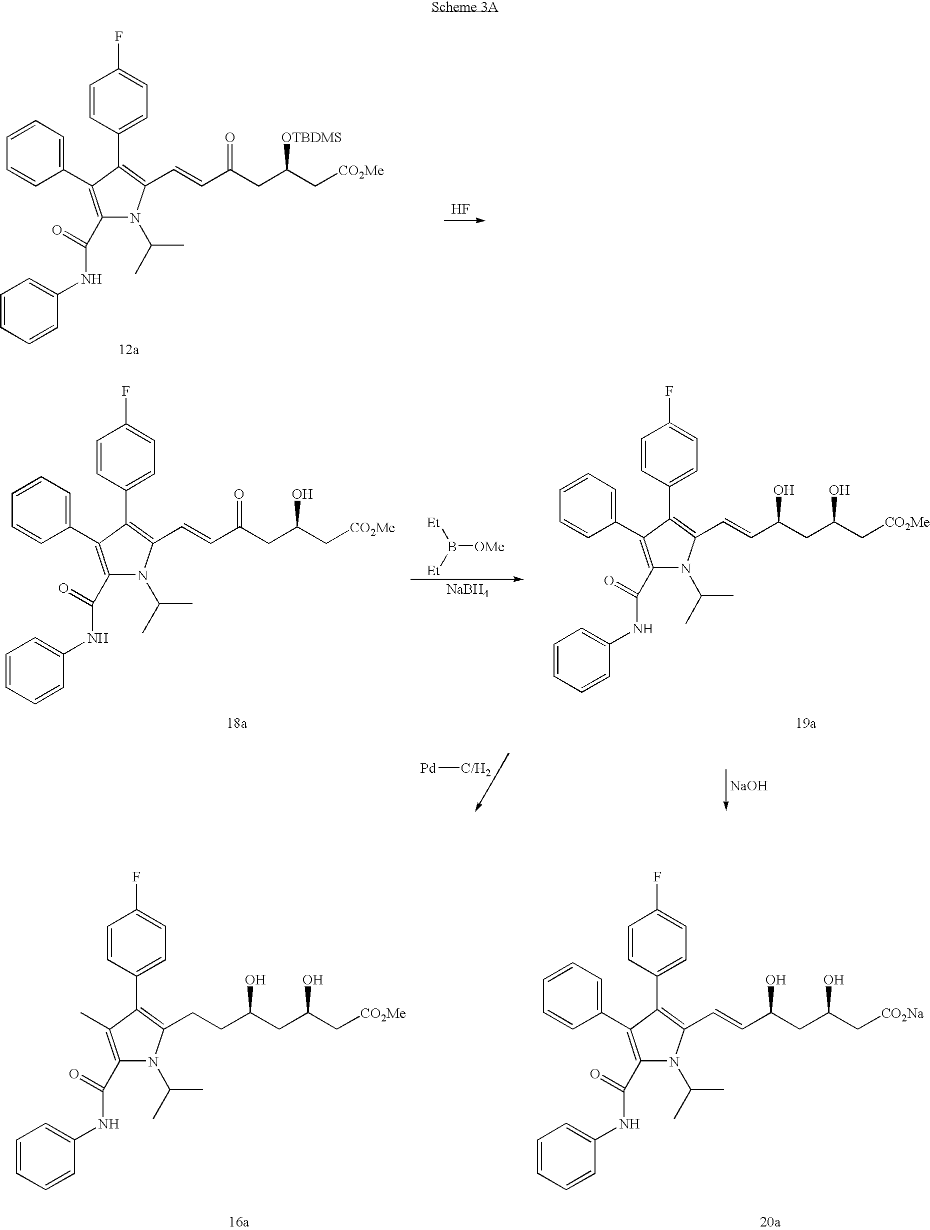

Compound 12a can be deprotected first to give compound 18a; stereoselective reduction of compound 18a gives compound 19a; hydrogenation of compound 19a affords compound 16a. Hydrolysis of compound 19a gives compound 20a.

Scheme 4 shows the preparation of compound 22, a mixture of stereoisomers wherein 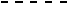
is absent, R¹ is isopropyl and R⁵ is phenyl-carbamoyl.

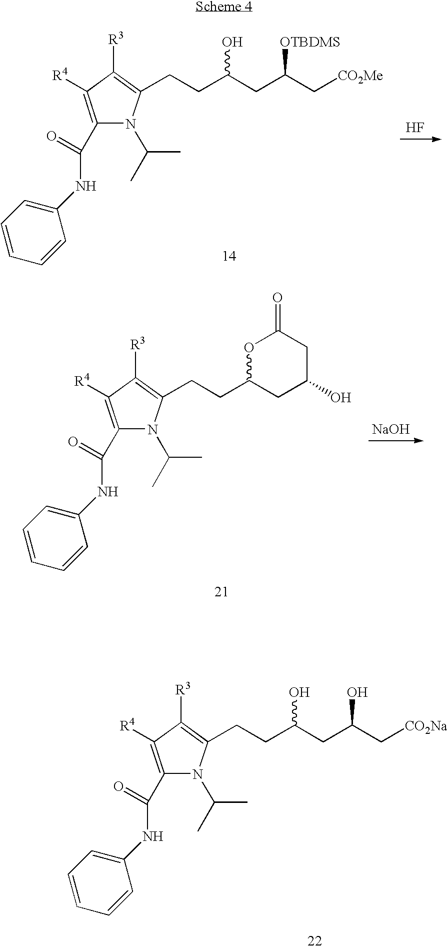

Scheme 4A shows a further example wherein R³ is para-fluorophenyl and R⁴ is phenyl.

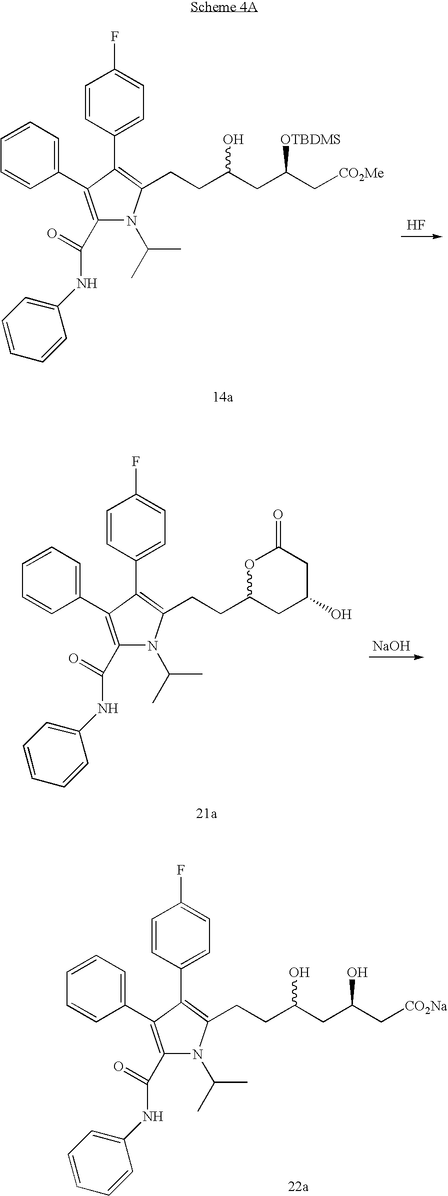

As shown in scheme 4A, the diastereomeric mixture 14a is deprotected to give a diastereomeric mixture 21a that is converted to a diastereomeric mixture 22a via hydrolysis reaction.

Scheme 5, which is exemplified in Example 21, shows an alternate route to the nitro alkene intermediate compound, useful for making compounds of the invention where R⁴ is, for example, isopropyl-.

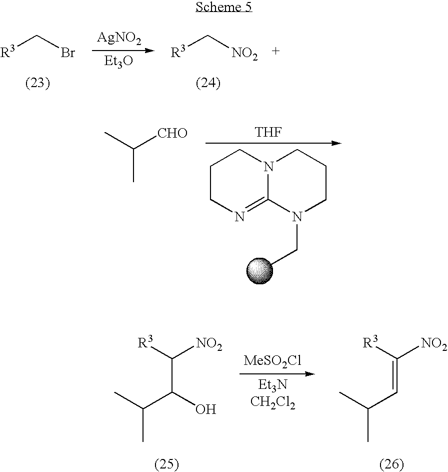

Scheme 6, which is exemplified in Example 22, shows a route to an aldehyde intermediate useful in the preparation of compounds-of the invention where R⁴ is, for example, methyl. In Scheme 6, R³ is for example 4-fluorophenyl.

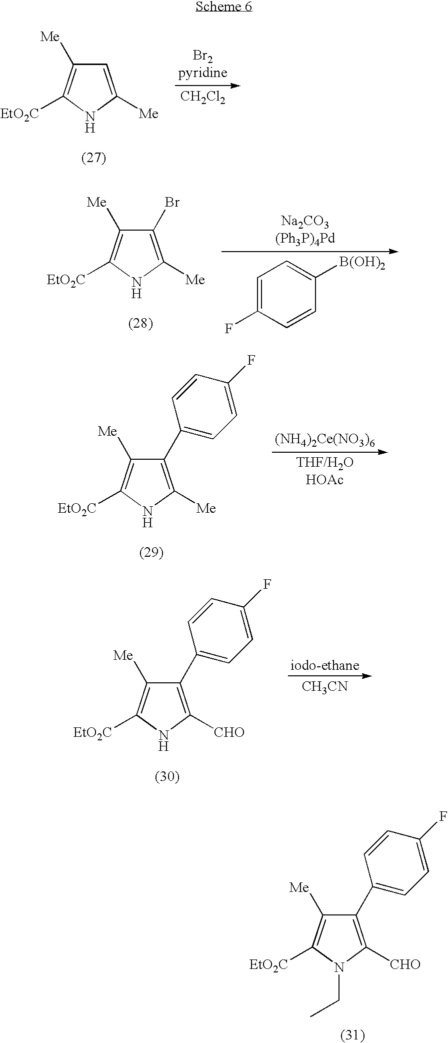

As shown in Scheme 6, bromination of the commercially available 3,5-dimethyl-1H-pyrrole-2-carboxylic acid ethyl ester yields the 4-bromo-3,5-dimethyl-1H-pyrrole-2-carboxylic acid ethyl ester. Displacement of the bromine with phenylboronic acid (Suzuki reaction), introduces a phenyl substituent into the pyrrole ring. Oxidation of the 5-methyl substituent with ceric ammonium nitrate introduces the aldehyde functionality. Additional intermediates may be obtained by alkylation of the pyrrole nitrogen with iodoethane, followed by saponification of the ethyl ester.

Scheme 7 shows the preparation of compounds of the invention wherein 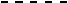
is absent and R⁵ is R⁶R⁷NC(O).

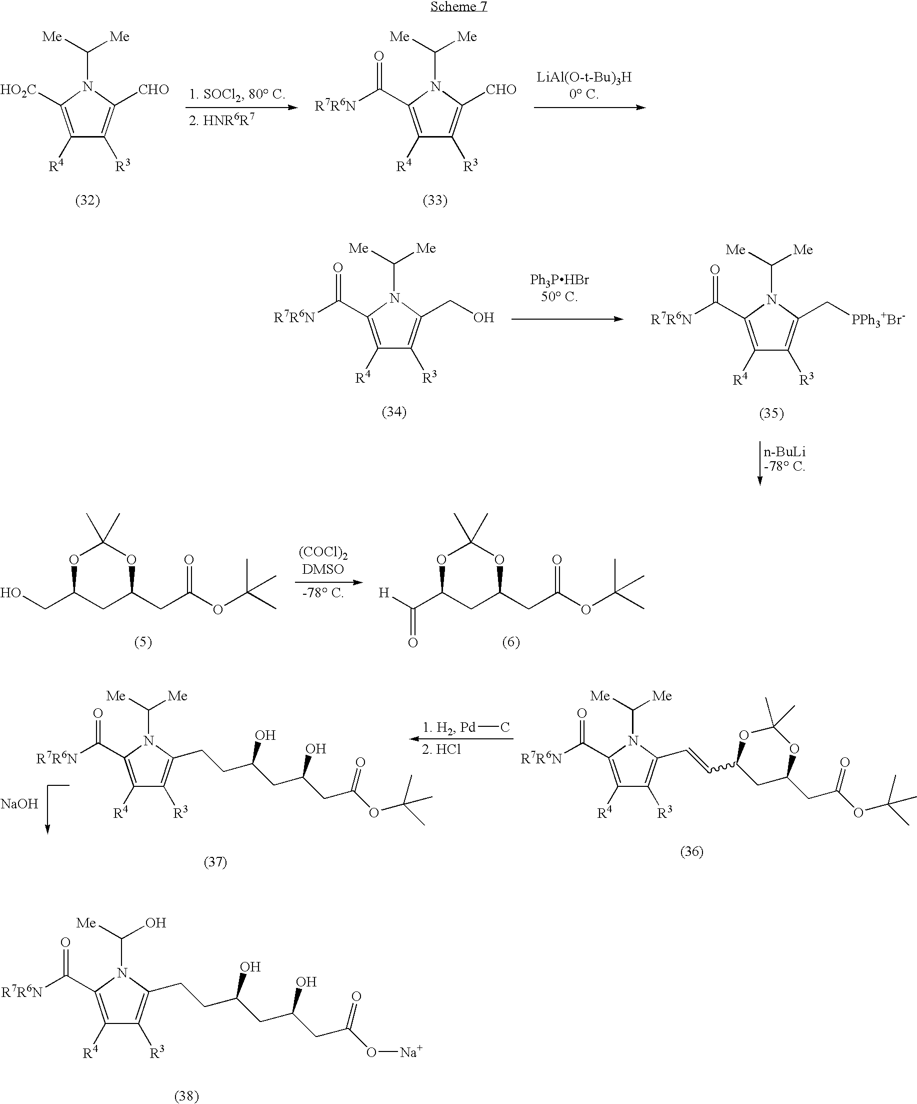

Scheme 7a shows a further example wherein R³ and R⁴ are each para-fluorophenyl-, and N, R⁶ and R⁷ taken together form a ring containing oxygen.

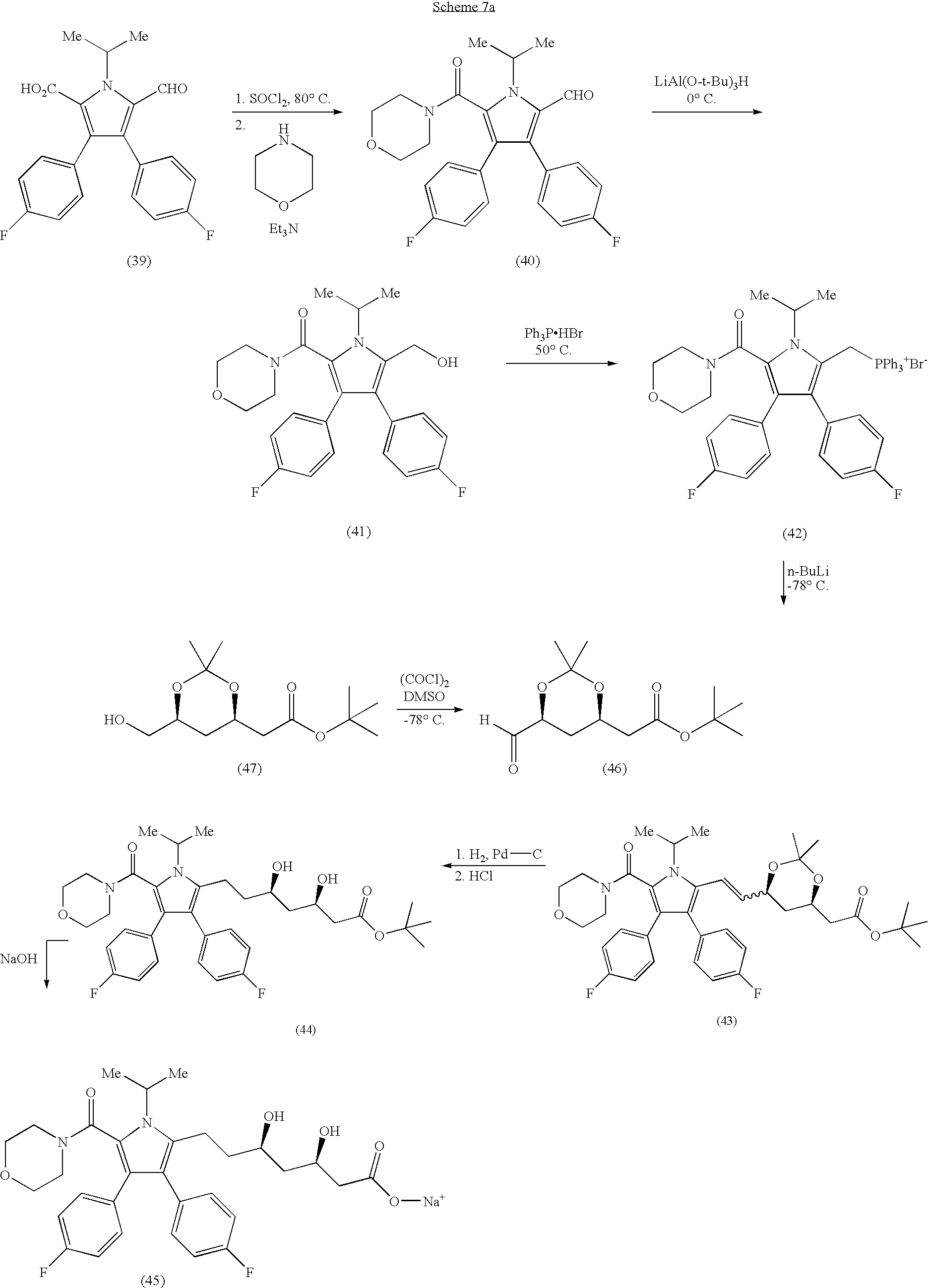

As shown in Scheme 7a, carboxylic acid (39) is converted to the amide (40) through the intermediacy of an acid chloride. The aldehyde of intermediate (40) is treated with lithium tri-t-butoxyaluminum hydride to afford the corresponding alcohol (41). Alcohol (41) is subsequently treated with triphenylphosphonium hydrobromide to afford Wittig intermediate (42). Aldehyde (46), prepared from alcohol (47) via Swern oxidation, is then coupled with Wittig reagent (42) in the presence of butyl lithium to provide olefin (43). Olefin (43) is hydrogenated over palladium on carbon catalyst and the acetonide protecting group is removed by treatment with HCl to provide diol (44). Finally, ester (44) is treated with aqueous NaOH to provide the corresponding carboxylic acid.

Scheme 8 exemplifies a further preparation of a Wittig intermediate which is exemplified in Example 27.

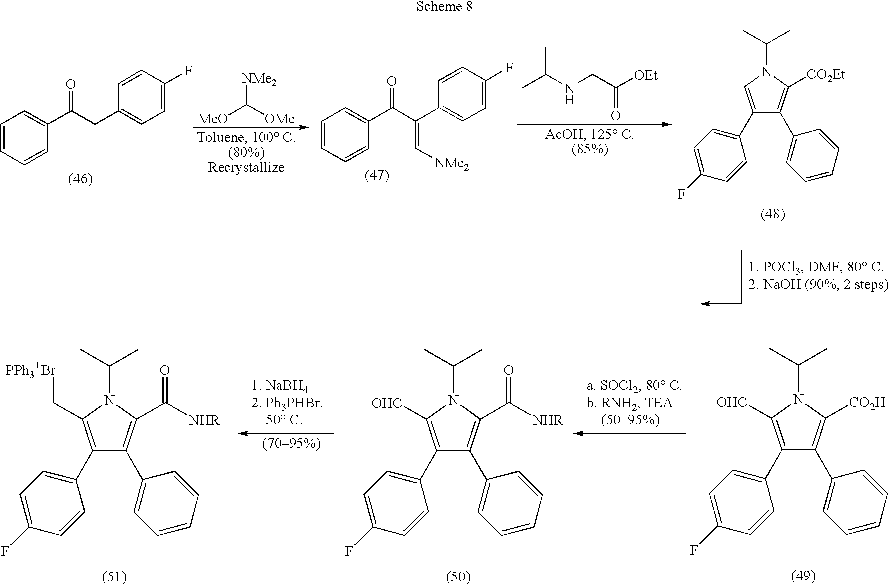

As shown in Scheme 8,2-(4-fluorophenyl)-1-phenylethanone (46) is treated with dimethylformamide dimethyl acetal at 100° C. to afford vinylogous amide (47). Treating vinylogous amide (47) with ethyl N-isopropylglycinate in AcOH at 125° C. provides pyrrole product (48). The pyrrole (48) is then treated with phosphorous oxychloride and dimethyl formamide to affect a formylation reaction. Subsequently, the ester is hydrolyzed to the corresponding carboxylic acid (49). The carboxylic acid is then converted to amide (50) via the intermediacy of an acid chloride. Finally, intermediate (50) is treated with sodium borohydride to afford an intermediate alcohol which is treated with triphenylphosphine hydrobromide to prepare phosphonium salt (51) which can be further elaborated as described in Scheme 7.

Scheme 9 shows a method of preparation of compound 57.

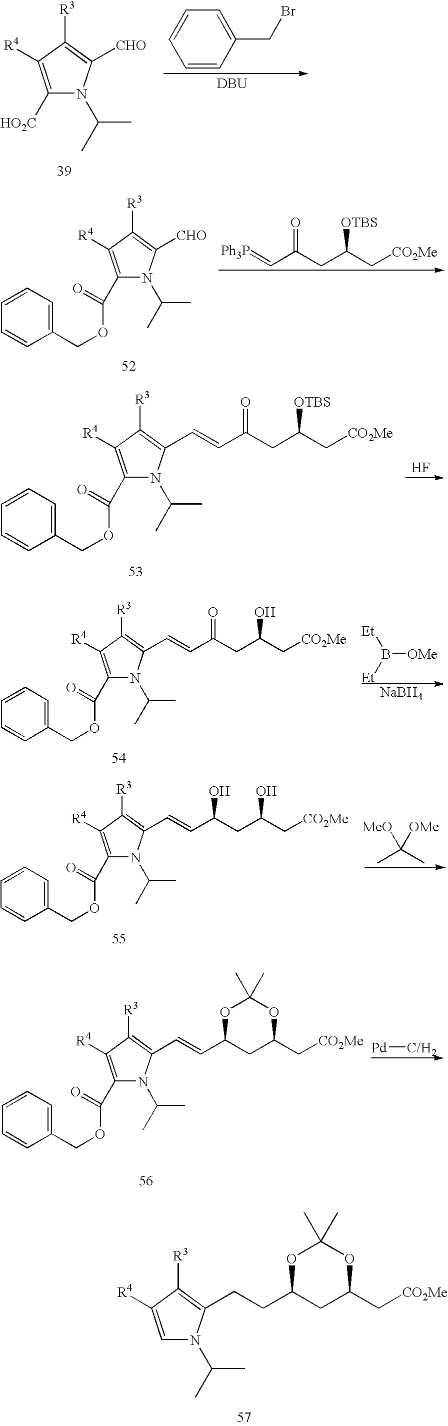

Scheme 9a shows an example wherein R³ and R⁴ are each para-fluorophenyl.

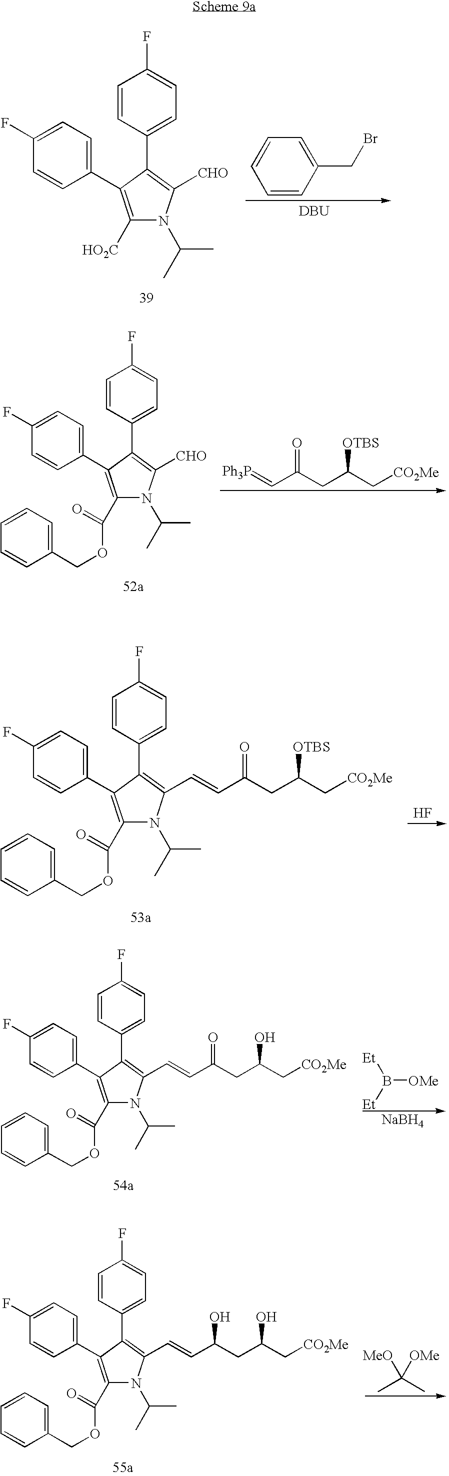

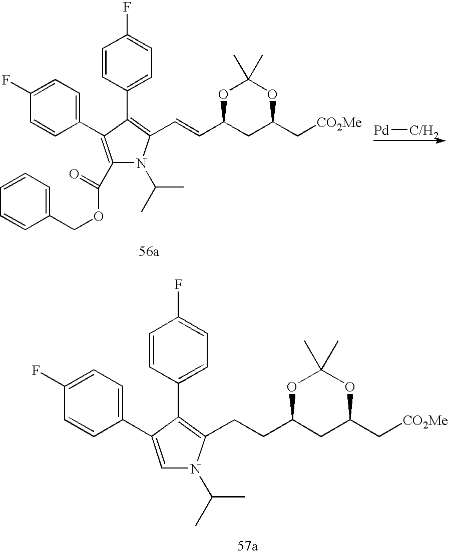

As shown in Scheme 9a, the acid-aldehyde 39 was reacted with benzylbromide in presence of DBU to give ester 52, which was coupled with the Wittig reagent shown to give compound 53. Deprotection of compound 53 with an aqueous HF solution gave keto-alcohol 54 in an excellent yield. Stereoselective reduction of keto-alcohol 54 afforded diol 55, which was protected as acetonide in compound 56. Hydrogenation and hydrogenolysis of compound 56 also resulted in decarboxylation of the carboxylic acid group to give compound 57.

Scheme 10, which is exemplified in Example 25, shows a method of preparation of compounds of the invention wherein R⁵ is R⁶R⁷NC(O)—, one of R⁶ and R⁷ is H and the other one of R⁶ and R⁷ is a substituted heteroaryl.

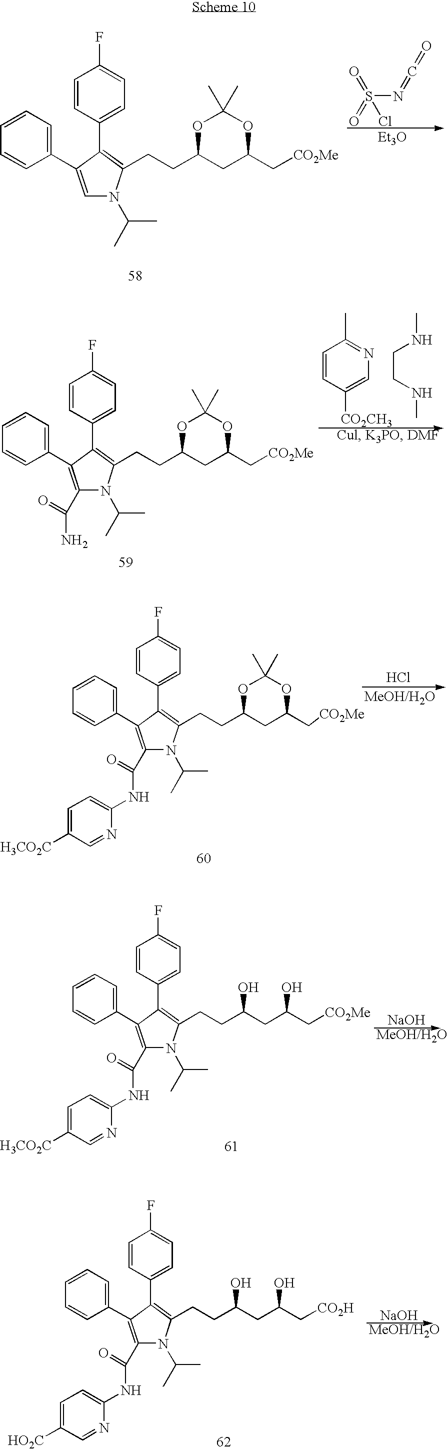

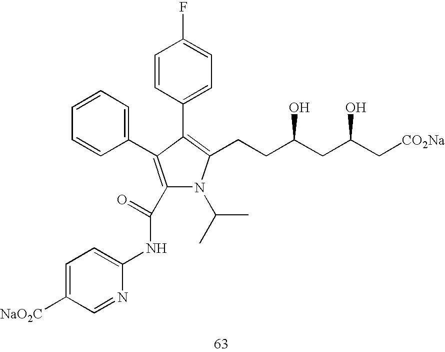

As shown in Scheme 10, the reaction of compound 58 with chlorosulfonylisocyanate in Et₂O gave amide 59. N-arylation of amide 59 with 6-iodo-nicotinic acid methyl ester under the catalytic condition described by Buchwald et al (J. Am. Chem. Soc. 2001, 123, 7727-7729) produced compound 60. Deprotection of the acetonide group and subsequent base hydrolysis afforded di-acid 62, which was converted to di-sodium salt 63 under standard conditions.

Scheme 11 shows a method of preparation of compounds of the invention wherein R⁵ is —(CH₂)ₙNR⁶R⁷, n is 1, one of R⁶ and R⁷ is H and the other one of R⁶ and R⁷ is COR′.

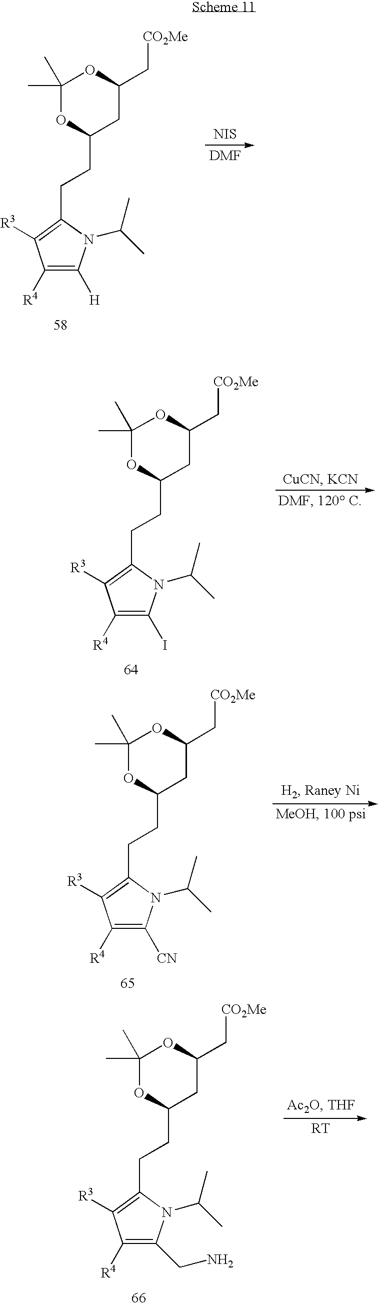

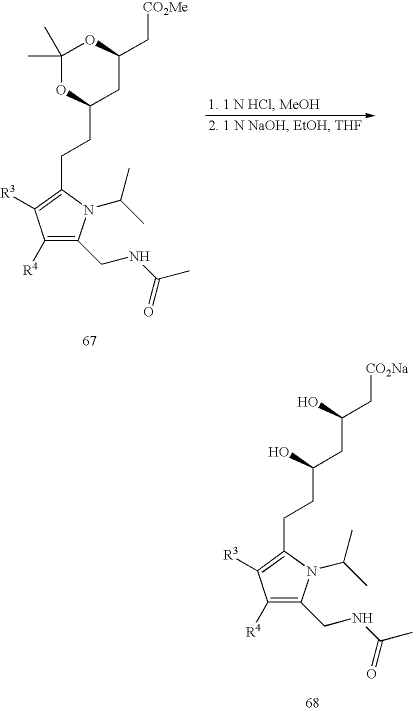

Scheme 11a shows a further example, which is exemplified in Example 26.

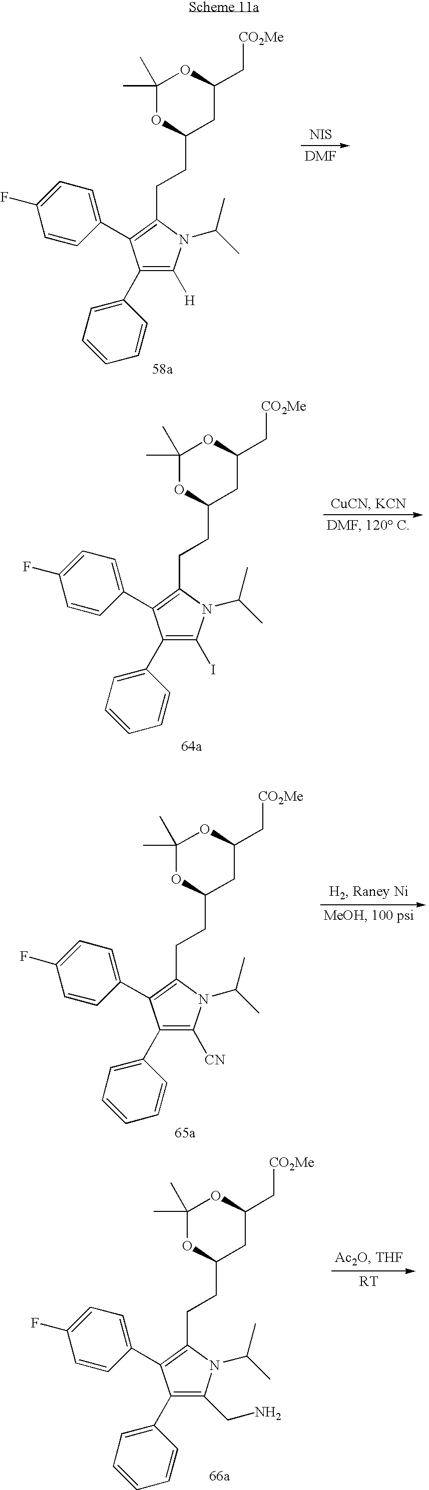

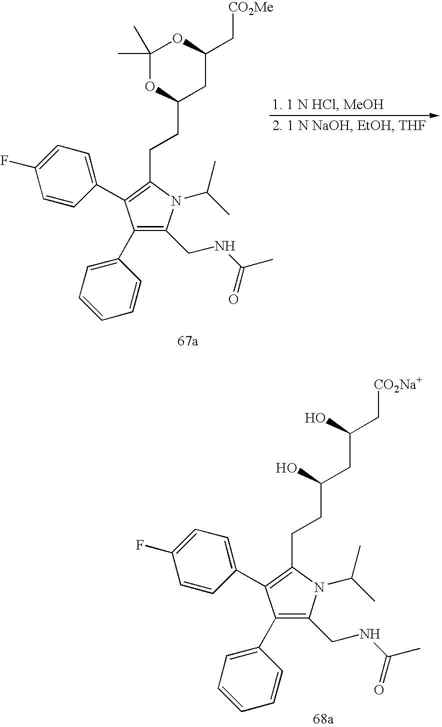

As shown in scheme 11, compound 58 was treated with NIS in DMF to afford the 2-iodopyrrol analog 64. This compound was in turn treated with CuCN and KCN in heated DMF to afford the cyano compound 65. Hydrogenation of 65 under 100 psi catalyzed by Raney nickel provided the primary amine 66. Compound 66 can be treated with any acyl chloride and/or acid anhydride such as acetic anhydride to afford product 67. Sequential deprotections by treating compound 67 with 1N HCl followed by 1 N NaOH provided the target compound 68.

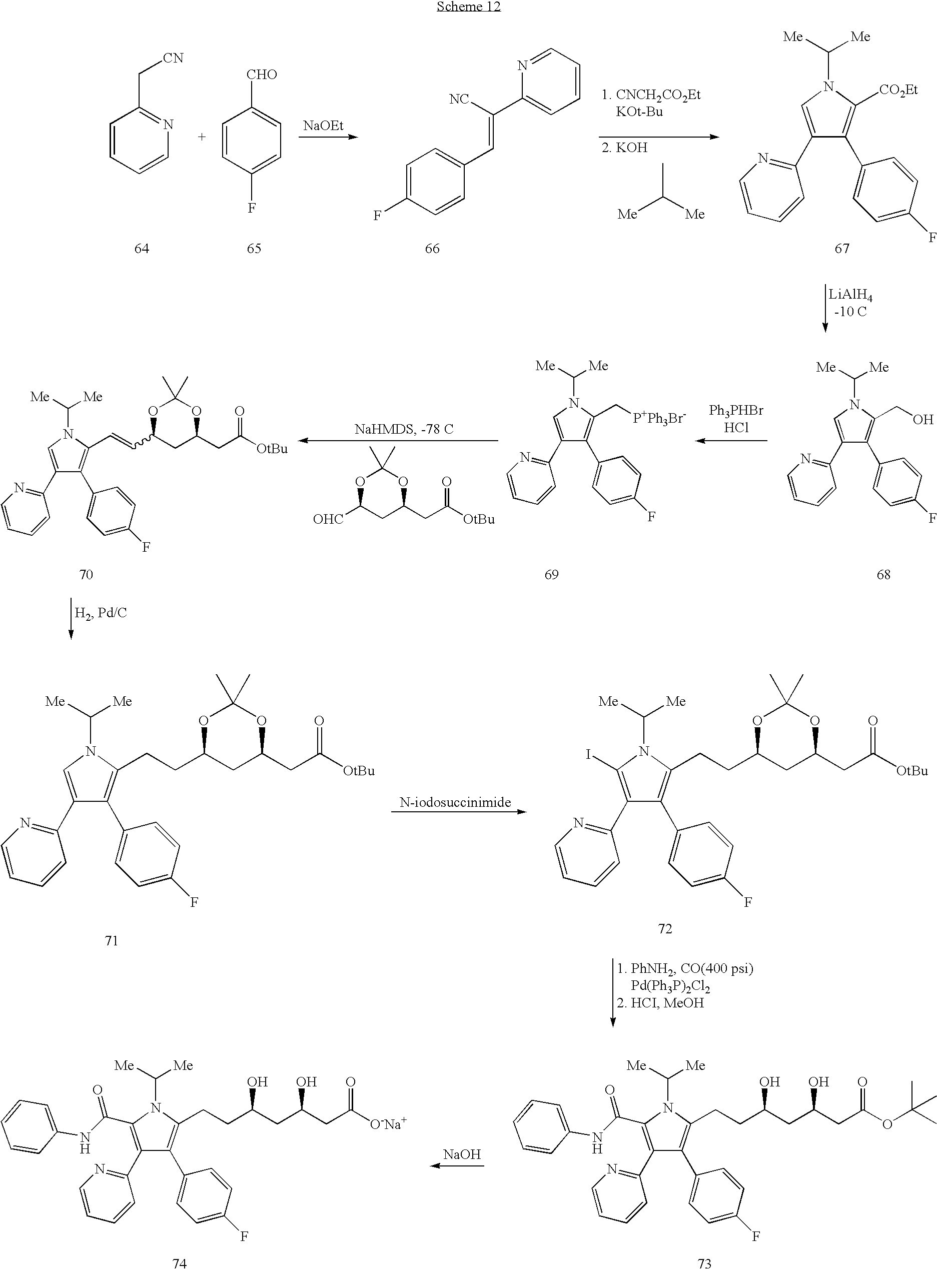

Scheme 12, which is exemplied in Example 61, illustrates the synthesis of compounds with a heterocyclic ring in the R₄ position. As shown, 4-fluorobenzaldehyde (65) was condensed with pyridine-2-yl-acetonitrile (64) in the presence of base to afford stillbene derivative (66). Intermediate (66) was converted to pyrrole (67) via cycloaddition with ethyl isocyanoacetate followed by alkylation with 2-iodopropane. The ester of intermediate (67) was then reduced to alcohol (68) which was converted to phosphonium salt (69) upon treatment with triphenylphosphine hydrobromide and HCl. Wittig olefination of phosphonium salt (69) afforded olefin (70) which was subjected to hydrogenation to give intermediate (71). Intermediate (71) was then treated with N-iodosuccinimide to give compound (72) which was subjected to a palladium catalyzed carbonylative coupling reaction with aniline to afford, after HCl treatment, compound (73). Finally, the ester of compound (73) was hydrolyzed by treatment with NaOH to give compound (74) which was isolated as a carboxylate salt.

Scheme 13 illustrates an alternate method of preparation of an aldehyde intermediate. 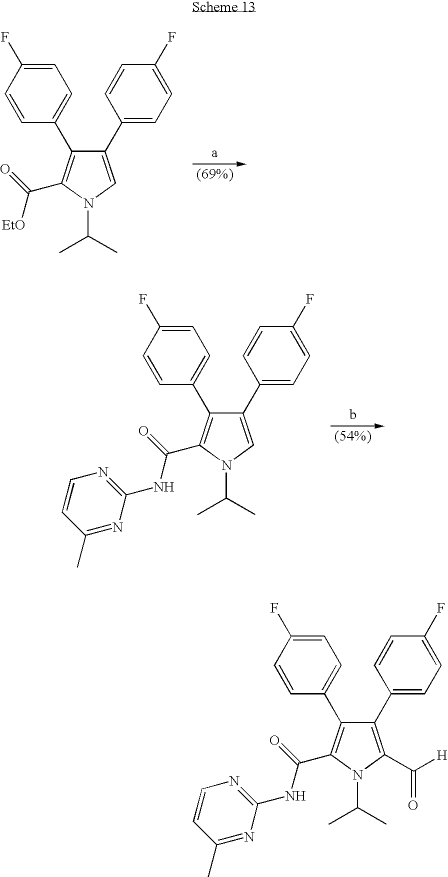
a.) 2-amino-4-methylpyrimidine, NaH, THF; b.) α,α-dichloromethyl methyl ether, TiCl₄, DCM.

### EXAMPLES

The following non-limiting Examples show how to carry out the present invention. The synthetic route of compounds of the present invention is not limited to the methods outlined below. It is assumed that one skilled in the art will be able to use the schemes outlined below to synthesize compounds claimed in this invention.

### Example 1

### (3R,5R)-7-[3-(4-fluoro-phenyl)-1-isopropyl-4-phenyl-5-phenylcarbamoyl-1H-pyrrol-2-yl]-3,5-dihydroxy-heptanoic Acid Sodium Salt

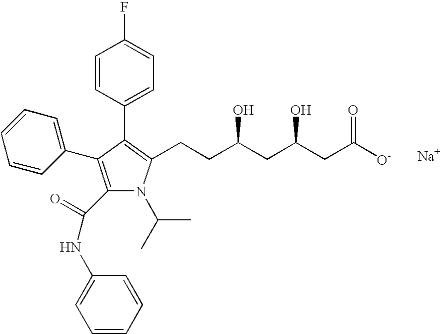 Step A

### 1-Fluoro-4-nitromethyl-benzene

To a suspension of silver nitrite (13.4 g, 87.3 mmol) in diethyl ether (150 mL), with stirring, was added 4-fluoro-benzylbromide (15 g, 79.4 mmol) dropwise in an ice-bath under a nitrogen atmosphere. After addition was complete, the mixture was allowed to warm to room temperature and stirred overnight. After TLC showed that the reaction was complete, the mixture was filtered. The filtrate was concentrated in vacuo to give a residue, the residue was purified by chromatography (0%-6% ethyl acetate in hexanes) to give 5.5 g (36%) of the desired product as a colorless syrup: MS(APCI⁻): m/z 154.0 (M-H); Anal. Calcd for C₇H₆F₁N₁O₂: C, 54.20; H, 3.90; N, 9.03. Found: C, 54.19; H, 3.87; N, 8.97.

Step B

### Benzylidene-butyl-amine

To a mixture of benzaldehyde (10.16 mL, 100 mmol) in benzene (100 mL) was added butylamine (9.86 mL, 100 mmol), dropwise, maintaining the reaction temperature below 30° C. After addition was complete, the mixture was heated at reflux for 1 h using a Dean-Stark condenser to collect ca. 1.8 mL water. The resulting mixture was concentrated in vacuo to give 16.1 g (100%) of the desired product as a colorless oil: MS(APCI⁺): m/z 162.1 (MH⁺); Anal. Calcd for C₁₁H₁₅N₁0.2H₂O0.2C₆H₆: C, 81.19; H, 9.27; N, 7.76. Found: C, 80.86; H, 9.21; N, 7.53.

Step C

### 1-Fluoro-4-(1-nitro-2-phenyl-vinyl)-benzene

To a solution of 1-fluoro-4-nitromethyl-benzene prepared from step A (5.14 g, 33.6 mmol) in acetic acid (8.4 mL) was added benzylidene-butyl-amine prepared from step B (5.4 g, 33.6 mmol). The mixture was stirred at room temperature overnight and a yellow crystalline solid formed. The solid was filtered, washed with water twice and dried in vacuo to give 5.1 g (63%) of the desired product as a yellow solid: mp 84-86° C.; MS(APCI⁻): m/z 243.0 (M−H); Anal. Calcd for C₁₄H₁₀F₁N₁O₂: C, 69.13; H, 4.14; N, 5.76. Found: C, 68.75; H, 4.03; N, 5.66.

Step D

### 4-(4-Fluoro-phenyl)-3-phenyl-1H-pyrrole-2-carboxylic Acid Ethyl Ester

To a mixture of 1-fluoro-4-(1-nitro-2-phenyl-vinyl)-benzene prepared from step C (4.9 g, 20.2 mmol) and ethyl isocyanoacetate (3.3 mL, 30.3 mmol) in THF (60 mL) was added DBU (4.52 mL, 30.3 mmol) slowly over 10 minutes under a nitrogen atmosphere. The resulting mixture was stirred at room temperature overnight and partitioned between ethyl acetate and water. The organic phase was separated and washed with water and brine, dried over Na₂SO₄ and filtered. The filtrate was concentrated in vacuo to give a residue, which was purified by chromatography (2%-12% ethyl acetate in hexanes) to give 2.3 g (37%) of the desired product as an off-white solid: mp 145-146° C.; MS(APCI⁻): m/z 308.1 (M−H); Anal. Calcd for C₁₉H₁₆F₁N₁O₂: C, 73.77; H, 5.21; N, 4.53. Found: C, 73.77; H, 5.11; N, 4.47.

Step E

### 4-(4-Fluoro-phenyl)-1-isopropyl-3-phenyl-1H-pyrrole-2-carboxylic Acid Ethyl Ester

To a mixture of pre-crushed potassium hydroxide (2 g, 35.6 mmol) in DMSO (17 mL) was added 4-(4-fluoro-phenyl)-3-phenyl-1H-pyrrole-2-carboxylic acid ethyl ester prepared from step D (2.2 g, 7.12 mmol). The mixture was stirred at room temperature under a nitrogen atmosphere for 45 min and then isopropyl iodide (2.1 mL, 21.4 mmol) was added dropwise. After addition was complete, the resulting mixture was stirred at room temperature for 45 min and partitioned between diethyl ether and water. The organic phase was separated and washed with water (three times) and brine, dried over Na₂SO₄ and filtered. The filtrate was concentrated in vacuo to give a residue, which was purified by chromatography (2%-10% ethyl acetate in hexanes) to give 2.13 g (85%) of the desired product as a white solid: mp 104-105° C.; MS(APCI⁺): m/z 352.1 (MH⁺); Anal. Calcd for C₂₂H₂₂F₁N₁O₂: C, 75.19; H, 6.31; N, 3.99. Found: C, 75.17; H, 6.40; N, 3.89.

Step F

### 4-(4-Fluoro-phenyl)-5-formyl-1-isopropyl-3-phenyl-1H-pyrrole-2-carboxylic Acid Ethyl Ester

To POCl₃ (0.67 mL, 7.18 mmol) was added anhydrous DMF (0.56 mL, 7.18 mmol) at −78° C. under a nitrogen atmosphere. After the mixture was stirred for 0.5 h, dichloroethane (2 mL), was added dropwise over 5 minutes followed by a solution of 4-(4-fluoro-phenyl)-1-isopropyl-3-phenyl-1H-pyrrole-2-carboxylic acid ethyl ester prepared from step E (2.1 g, 5.98 mmol) in dichloroethane (2 mL) dropwise over 10 minutes. At the end of the addition the cooling bath was removed and the reaction was heated at reflux for 1 h. The mixture was cooled, to room temperature, and then cooled in an ice bath. Saturated sodium acetate solution (5 mL) was added slowly, and the ice bath was removed. The solution was again brought to reflux for 1 h and then partitioned between ethyl acetate and water. The organic phase was separated and washed with water and brine, dried over Na₂SO₄ and filtered. The filtrate was concentrated in vacuo to give a residue, which was purified by chromatography (2%-10% ethyl acetate in hexanes) to give 1.5 g (66%) of the desired product as a white solid: mp 88-90° C.; MS(APCI⁺): m/z 380.2 (MH⁺); Anal. Calcd for C₃₃H₂₂F₁N₁O₃: C, 72.81; H, 5.84; N, 3.69. Found: C, 72.80; H, 5.76; N, 3.65.

Step G

### 4-(4-Fluoro-phenyl)-5-formyl-1-isopropyl-3-phenyl-1H-pyrrole-2-carboxylic Acid

To a solution of 4-(4-fluoro-phenyl)-5-formyl-1-isopropyl-3-phenyl-1H-pyrrole-2-carboxylic acid ethyl ester prepared from step F (1.45 g, 3.83 mmol) in methanol (20 mL) was added a solution of sodium hydroxide (0.61 g, 15.3 mmol) in water (3 mL). The mixture was stirred at 60° C. for 2 h. TLC showed that the reaction was complete. The mixture was then cooled, and partitioned between ethyl acetate and 1N HCl solution. The organic phase was separated and washed with water and brine, dried over Na₂SO₄ and filtered. The filtrate was concentrated in vacuo to give 1.34 g (100%) of the desired product as a white solid: mp 219-220° C.; MS(APCI⁻): m/z 350.1 (M−H); Anal. Calcd for C₂₁H₁₈F₁N₁O₃: C, 71.78; H, 5.16; N, 3.99. Found: C, 71.54; H, 5.24; N, 3.81.

Step H

### 4-(4-Fluoro-phenyl)-5-formyl-1-isopropyl-3-phenyl-1H-pyrrole-2-carboxylic Acid Phenylamide

To a mixture of 4-(4-fluoro-phenyl)-5-formyl-1-isopropyl-3-phenyl-1H-pyrrole-2-carboxylic acid prepared from step G (1.33 g, 3.79 mmol) in dry THF (20 mL) in an ice bath under a nitrogen atmosphere was added one drop of DMF followed by oxalyl chloride (0.4 mL, 4.55 mmol). The mixture was stirred for 1 h the then stirred at room temperature for 2 h. TLC showed that the reaction was complete. The mixture was cooled in an ice bath and aniline (0.35 mL, 3.79 mmol) was added followed by triethylamine (1.06 mL, 7.58 mmol). The mixture was stirred at room temperature overnight and partitioned between ethyl acetate and water. The organic phase was separated and washed with 1N HCl, NaHCO₃ and brine, dried over Na₂SO₄ and filtered. The filtrate was concentrated in vacuo to give a residue, which was purified by chromatography (2%-20% ethyl acetate in hexanes) to give 0.75 g (46%) of the desired product as an off-white solid: mp 222-224° C.; MS(APCI⁺): m/z 427.1 (MH⁺); Anal. Calcd for C₂₇H₂₃F₁N₂O₂0.25EtOAc: C, 74.41; H, 5.59; N, 6.19. Found: C, 74.13; H, 5.29; N, 6.51.

Step I

### (3R)-3-(tert-Butyl-dimethyl-silanyloxy)-7-[3-(4-fluoro-phenyl)-1-isopropyl-4-phenyl-5-phenylcarbamoyl-1H-pyrrol-2-yl]-5-oxo-hept-6-enoic Acid Methyl Ester

To a mixture of 4-(4-fluoro-phenyl)-5-formyl-1-isopropyl-3-phenyl-1H-pyrrole-2-carboxylic acid phenylamide prepared from step H (726 mg, 1.70 mmol) in toluene (20 mL) at room temperature under a nitrogen atmosphere was added wittig reagent [3-(tert-butyl-dimethyl-silanyloxy)-5-oxo-6-(triphenyl-phosphanylidene)-hexanoic acid methyl ester] (1.38 g, 2.58 mmol). The mixture was heated at reflux for 40 h and then concentrated in vacuo to give a residue, which was purified by chromatography (2%-15% ethyl acetate in hexanes) to give 0.75 g (65%) of the desired product as a yellow foam: mp 62-64° C.; MS(APCI⁺): m/z 683.2 (MH⁺); Anal. Calcd for C₄₀H₄₇F₁N₂O₅Si₁: C, 70.35; H, 6.94; N, 4.10. Found: C, 70.32; H, 7.07; N, 4.00.

Step J

### (3R)-3-(tert-Butyl-dimethyl-silanyloxy)-7-[3-(4-fluoro-phenyl)-1-isopropyl-4-phenyl-5-phenylcarbamoyl-1H-pyrrol-2-yl]-5-oxo-heptanoic Acid Methyl Ester and (3R)-3-(tert-butyl-dimethyl-silanyloxy)-7-[3-(4-fluoro-phenyl)-1-isopropyl-4-phenyl-5-phenylcarbamoyl-1H-pyrrol-2-yl]-5-hydroxyl-heptanoic Acid Methyl Ester

To a solution of (3R)-3-(tert-butyl-dimethyl-silanyloxy)-7-[3-(4-fluoro-phenyl)-1-isopropyl-4-phenyl-5-phenylcarbamoyl-1H-pyrrol-2-yl]-5-oxo-hept-6-enoic acid methyl ester prepared from step 1 (610 mg, 0.9 mmol) in THF (30 mL) was added 10% palladium on activated carbon (100 mg). The mixture was stirred at room temperature under a hydrogen atmosphere for 3 h. TLC showed that the reaction was complete. The mixture was filtered through celite. The filtrate was concentrated in vacuo to give a residue, which was purified by chromatography (2%-15% ethyl acetate in hexanes) to give a first fraction of 0.32 g (52%) of light yellow foam: mp 53-55° C.; MS(APCI⁺): m/z 685.2 (MH⁺); Anal. Calcd for C₄₀H₄₉F₁N₂O₅Si₁: C, 70.15; H, 7.21; N, 4.09. Found: C, 70.27; H, 7.46; N, 4.03; and a second fraction of 0.24 g (39%) of light yellow foam: mp 63-65° C.; MS(APCI⁺): m/z 687.2 (MH⁺); Anal. Calcd for C₄₀H₅₁F₁N₂O₅Si₁: C, 69.94; H, 7.48; N, 4.08. Found: C, 69.98; H, 7.76; N, 3.99.

Step K

### (3R)-7-[3-(4-Fluoro-phenyl)-1-isopropyl-4-phenyl-5-phenylcarbamoyl-1H-pyrrol-2-yl]-3-hydroxy-5-oxo-heptanoic Acid Methyl Ester

To a solution of (3R)-3-(tert-butyl-dimethyl-silanyloxy)-7-[3-(4-fluoro-phenyl)-1-isopropyl-4-phenyl-5-phenylcarbamoyl-1H-pyrrol-2-yl]-5-oxo-heptanoic acid methyl ester prepared from step J (300 mg, 0.44 mmol) in acetonitrile (1.6 mL) was added dropwise a hydrogen fluoride solution (1:19 48% HF:acetonitrile, 6.5 mL) in an ice bath under a nitrogen atmosphere. The mixture was stirred at room temperature for 1 h. TLC showed that the reaction was complete. The mixture was partitioned between ethyl acetate and water. The organic phase was separated and washed with NaHCO₃ and brine, dried over Na₂SO₄ and filtered. The filtrate was concentrated in vacuo to give 0.25 g (100%) of the desired product as an off-white foam: mp 75-77° C.; MS(APCI⁺): m/z 571.2 (MH⁺); Anal. Calcd for C₃₄H₃₅F₁N₂O₅: C, 71.56; H, 6.18; N, 4.91. Found: C, 71.48; H, 6.37; N, 4.72.

Step L

### (3R,5R)-7-[3-(4-Fluoro-phenyl)-1-isopropyl-4-phenyl-5-phenylcarbamoyl-1H-pyrrol-2-yl]-3,5-dihydroxy-heptanoic Acid Methyl Ester

To a mixture of (3R)-7-[3-(4-fluoro-phenyl)-1-isopropyl-4-phenyl-5-phenylcarbamoyl-1H-pyrrol-2-yl]-3-hydroxy-5-oxo-heptanoic acid methyl ester prepared from step K (246 mg, 0.43 mmol) in THF (5.6 mL) and methanol (1.4 mL),was added dropwise a solution of 1M diethyl-methoxy-borane in THF (0.43 mL) at −78° C. under a nitrogen atmosphere. The mixture was stirred for 0.5 h and then sodium borohydride (21.2 mg, 0.56 mmol) was added in portions. After stirring for 2 h, 2 drops of acetic acid was added. The mixture was partitioned between ethyl acetate and water. The organic phase was separated and washed with NaHCO₃ and brine, dried over Na₂SO₄ and filtered. The filtrate was concentrated in vacuo to give a residue, which was dissolved in warm methanol and concentrated in vacuo again to give a residue, which was purified by chromatography (10%-50% ethyl acetate in hexanes) to give 206 mg (84%) of the desired product as a white foam: mp 154-157° C.; MS(APCI⁺): m/z 573.2 (MH⁺); Anal. Calcd for C₃₄H₃₇F₁N₂O₅0.3EtOAc: C, 70.57; H, 6.63; N, 4.68. Found: C, 70.43; H, 6.37; N, 4.66.

Step M

### (3R,5R)-7-[3-(4-fluoro-phenyl)-1-isopropyl-4-phenyl-5-phenylcarbamoyl-1H-pyrrol-2-yl]-3,5-dihydroxy-heptanoic Acid Sodium Salt

To a mixture of (3R,5R)-7-[3-(4-fluoro-phenyl)-1-isopropyl-4-phenyl-5-phenylcarbamoyl-1H-pyrrol-2-yl]-3,5-dihydroxy-heptanoic acid methyl ester prepared from step L (190 mg, 0.33 mmol), in a solution of absolute ethanol (2.2 mL) and water (1 mL), was added 1N aqueous sodium hydroxide solution (0.33 mL) at room temperature. The mixture was stirred for 1 h and then concentrated in vacuo to give a residue, which was dissolved in a solution of 20% methanol in methylene chloride and filtered. The filtrate was concentrated in vacuo to give a solid. The solid was triturated with diethyl ether and filtered and dried in vacuo to give 190 mg (99%) of the desired product as a white solid: mp 239-241° C.; MS(APCI⁺): m/z 559.2 (MH⁺); Anal. Calcd for C₃₃H₃₄F₁N₂O₅Na₁1.0H₂O0.25EtOH: C, 65.94; H, 6.19; N, 4.59. Found: C, 65.82; H, 5.94; N, 4.53.

### Example 2

### (3R,5S)-7-[3-(4-Fluoro-phenyl)-1-isopropyl-4-phenyl-5-phenylcarbamoyl-1H-pyrrol-2-yl]-3,5-dihydroxy-hept-6-enoic Acid Sodium Salt

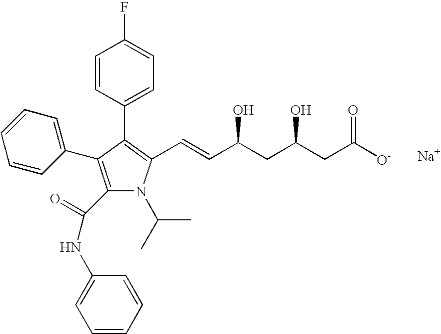 Step A

### (3R)-7-[3-(4-Fluoro-phenyl)-1-isopropyl-4-phenyl-5-phenylcarbamoyl-1H-pyrrol-2-yl]-3-hydroxy-5-oxo-hept-6-enoic Acid Methyl Ester

To a solution of (3R)-3-(tert-butyl-dimethyl-silanyloxy)-7-[3-(4-fluoro-phenyl)-1-isopropyl-4-phenyl-5-phenylcarbamoyl-1H-pyrrol-2-yl]-5-oxo-hept-6-enoic acid methyl ester prepared from Example 1, step I (120 mg, 0.176 mmol) in acetonitrile (0.64 mL) cooled in an ice bath was added dropwise a hydrogen fluoride solution (1:19 48% HF:acetonitrile, 2.6 mL) under a nitrogen atmosphere. The mixture was stirred at room temperature for 1 h. TLC showed that the reaction was complete. The mixture was partitioned between ethyl acetate and water. The organic phase was separated and washed with NaHCO₃ and brine, dried over Na₂SO₄ and filtered. The filtrate was concentrated in vacuo to give 100 mg (100%) of the desired product as a light yellow foam: mp 72-74° C.; MS(APCI⁺): m/z 569.2 (MH⁺); Anal. Calcd for C₃₄H₃₃F₁N₂O₅: C, 71.82; H, 5.85; N, 4.93. Found: C, 71.17; H, 5.76; N, 4.61.

Step B

### (3R,5S)-7-[3-(4-Fluoro-phenyl)-1-isopropyl-4 phenyl-5-phenylcarbamoyl-1H-pyrrol-2-yl]-3,5-dihydroxy-hept-6-enoic Acid Methyl Ester

To a mixture of (3R)-7-[3-(4-fluoro-phenyl)-1-isopropyl-4-phenyl-5-phenylcarbamoyl-1H-pyrrol-2-yl]-3-hydroxy-5-oxo-hept-6-enoic acid methyl ester prepared from step A (100 mg, 0.176 mmol) in THF (2.3 mL) and methanol (0.6 mL) was added dropwise a solution of 1M diethyl-methoxy-borane in THF (0.19 mL) at −78° C. under a nitrogen atmosphere. The mixture was stirred for 0.5 h and then sodium borohydride (8.6 mg, 0.23 mmol) was added in portions. After stirring for 2 h, one drop of acetic acid was added. The mixture was partitioned between ethyl acetate and water. The organic phase was separated and washed with NaHCO₃ and brine, dried over Na₂SO₄ and filtered. The filtrate was concentrated in vacuo to give a residue, which was dissolved in warm methanol and concentrated in vacuo again to give a residue, which was purified by chromatography (10%-50% ethyl acetate in hexanes) to give 45 mg (45%) of the desired product as a light yellow solid: mp 154-155° C.; MS(APCI⁺): m/z 571.2 (MH⁺); Anal. Calcd for C₃₄H₃₅F₁N₂O₅: C, 71.56; H, 6.18; N, 4.91. Found: C, 71.59; H, 6.18; N, 4.84.

Step C

### (3R,5R)-7-[3-(4-fluoro-phenyl)-1-isopropyl-4-phenyl-5-phenylcarbamoyl-1H-pyrrol-2-yl]-3,5-dihydroxy-hept-6-enoic Acid Sodium Salt

To a mixture of (3R,5R)-7-[3-(4-fluoro-phenyl)-1-isopropyl-4-phenyl-5-phenylcarbamoyl-1H-pyrrol-2-yl]-3,5-dihydroxy-hept-6-enoic acid methyl ester prepared from step B (22 mg, 0.039 mmol) in a solution of absolute ethanol (0.5 mL) and water (0.5 mL) was added 1N aqueous sodium hydroxide solution (0.039 mL) at room temperature. The mixture was stirred for 1 h and then concentrated in vacuo to give a residue, which was dissolved in a solution of 10% methanol in methylene chloride and filtered. The filtrate was concentrated in vacuo to give a solid. The solid was triturated with diethyl ether and filtered and dried in vacuo to give 22 mg (99%) of the desired product as an off-white solid: mp 239-241° C.; MS(APCI⁻): m/z 556.2 (M−H); Anal. Calcd for C₃₃H₃₂F₁N₂O₅Na₁.25H₂O: C, 65.94; H, 5.78; N, 4.66. Found: C, 66.05; H, 5.40; N, 4.58.

### Example 3

### (3R)-7-[3-(4-Fluoro-phenyl)-1-isopropyl-4-phenyl-5-phenylcarbamoyl-1H-pyrrol-2-yl]-3,5-dihydroxy-heptanoic Acid Sodium Salt

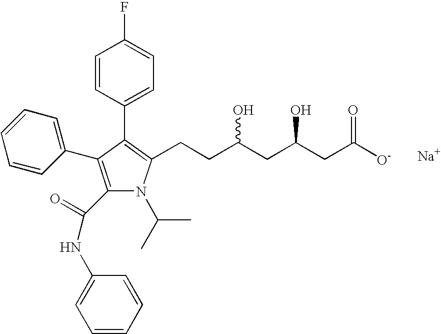 Step A

### (4R)-4-(4-Fluoro-phenyl)-5-[2-(4-hydroxy-6-oxo-tetrahydro-pyran-2-yl)-ethyl]-1-isopropyl-3-phenyl-1H-pyrrole-2-carboxylic Acid Phenylamide

To a solution of (3R)-3-(tert-butyl-dimethyl-silanyloxy)-7-[3-(4-fluoro-phenyl)-1-isopropyl-4-phenyl-5-phenylcarbamoyl-1H-pyrrol-2-yl]-5-hydroxyl-heptanoic acid methyl ester prepared from Example 1, step J (220 mg, 0.32 mmol) in acetonitrile (1.1 mL) was added dropwise a hydrogen fluoride solution (1:19 48% HF:acetonitrile, 4.6 mL) in an ice bath under a nitrogen atmosphere. The mixture was stirred at room temperature for 1 h. TLC showed that the reaction was complete. The mixture was partitioned between ethyl acetate and water. The organic phase was separated and washed with NaHCO₃ and brine, dried over Na₂SO₄ and filtered. The filtrate was concentrated in vacuo to give a residue, which was purified by chromatography (20%-60% ethyl acetate in hexanes) to give 120 mg (69%) of the desired product as a white solid: mp 128-129° C.; MS(APCI⁺): m/z 541.2 (MH⁺); Anal. Calcd for C₃₃H₃₃F₁N₂O₄0.5H₂O0.5EtOAc: C, 70.81; H, 6.45; N, 4.72. Found: C, 70.93; H, 6.15; N, 4.76.

Step B

### (3R)-7-[3-(4-Fluoro-phenyl)-1-isopropyl-4-phenyl-5-phenylcarbamoyl-1H-pyrrol-2-yl]-3,5-dihydroxy-heptanoic Acid Sodium Salt

To a mixture of (4R)-4-(4-fluoro-phenyl)-5-[2-(4-hydroxy-6-oxo-tetrahydro-pyran-2-yl)-ethyl]-1-isopropyl-3-phenyl-1H-pyrrole-2-carboxylic acid phenylamide prepared from step A (100 mg, 0.185 mmol) in a solution of absolute ethanol (0.5 mL) and water (0.5 mL) was added 1N aqueous sodium hydroxide solution (0.185 mL) at room temperature. The mixture was stirred for 1 h and then concentrated in vacuo to give a residue, which was dissolved in a solution of 10% methanol in methylene chloride and filtered. The filtrate was concentrated in vacuo to give a solid. The solid was triturated with diethyl ether and filtered and dried in vacuo to give 100 mg (99%) of the desired product as a white solid: mp 240-242° C.; MS(APCI⁺): m/z 559.2 (MH⁺); Anal. Calcd for C₃₃H₃₄F₁N₂ O₅Na₁1.5H₂O: C, 65.23; H, 6.14; N, 4.61. Found: C, 65.30; H, 5.77; N, 4.45.

### Example 4

### (3R,5R)-7-[3-(4-Fluoro-phenyl)-1-isopropyl-4-phenyl-5-(4-sulfamoyl-phenylcarbamoyl)-1H-pyrrol-2-yl]-3,5-dihydroxy-heptanoic Acid Sodium Salt

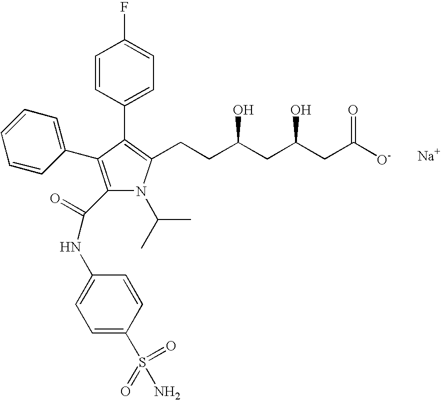

Example 4 was made by a method analogous to Example 1. mp 265-267° C.; MS(APCI⁻): m/z 638.3 (M−H); Anal. Calcd for C₃₃H₃₅F₁N₃O₇S₁Na₁.2.5H₂O: C, 56.24; H, 5.72; N, 5.96. Found: C, 55.92; H, 5.52; N, 5.70.

### Example 5

### (3R,5S)-7-[3-(4-Fluoro-phenyl)-1-isopropyl-4-phenyl-5-(4-sulfamoyl-phenylcarbamoyl)-1H-pyrrol-2-yl]-3,5-dihydroxy-hept-6-enoic Acid Sodium Salt

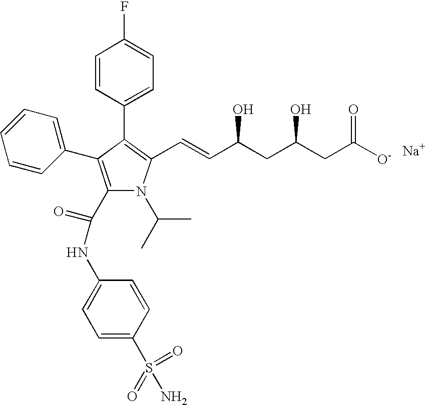

To a mixture of (3R,5R)-7-[3-(4-fluoro-phenyl)-1-isopropyl-4-phenyl-5-(4-sulfamoyl-phenylcarbamoyl)-1H-pyrrol-2-yl]-3,5-dihydroxy-hept-6-enoic acid methyl ester prepared from Example 4, Step D (89.3 mg, 0.137 mmol) in a solution of absolute ethanol (0.5 mL) and water (0.5 mL) was added 1N aqueous sodium hydroxide solution (0.137 mL) at room temperature. The mixture was stirred for 1 h and then concentrated in vacuo to give a residue, which was dissolved in a solution of 30% methanol in methylene chloride and filtered. The filtrate was concentrated in vacuo to give a solid. The solid was triturated with diethyl ether and filtered and dried in vacuo to give 90 mg (100%) of the desired product as a light yellow solid: mp 255-256° C.; MS(APCI⁻): m/z 634.2 (M−H); Anal. Calcd for C₃₃H₃₃F₁N₃O₇S₁Na₁.1.0H₂O: C, 58.66; H, 5.22; N, 6.22. Found: C, 58.54; H, 5.28; N, 6.10.

### Example 6

### (3R,5R)-7-[3-(4-Fluoro-phenyl)-5-(4-fluoro-phenylcarbamoyl)-1-isopropyl-4-phenyl-1H-pyrrol-2-yl]-3,5-dihydroxy-heptanoic Acid Sodium Salt

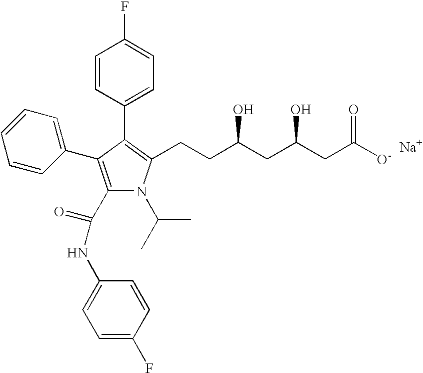

Example 6 was made by a method analogous to Example 1. MS(APCI⁻): m/z 575.3 (M−H); Anal. Calcd for C₃₃H₃₃F₂N₂O₅Na₁.0.5H₂O.0.35CH₂Cl₂: C, 62.85; H, 5.49; N, 4.40. Found: C, 62.54; H, 5.09; N, 4.28.

### Example 7

### (3R,5S)-7-[3-(4-Fluoro-phenyl)-5-(4-fluoro-phenylcarbamoyl)-1-isopropyl-4-phenyl-5-phenylcarbamoyl-1H-pyrrol-2-yl]-3,5-dihydroxy-hept-6-enoic Acid Sodium Salt

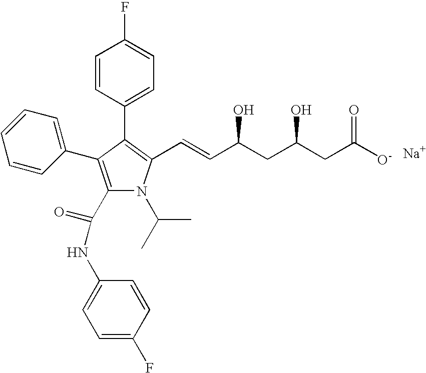

Example 7 was made by a method analogous to Example 2. MS(APCI⁻): m/z 575.3 (M−H); Anal. Calcd for C₃₃H₃₁F₂N₂O₅Na₁.2.0H₂O: C, 62.65; H, 5.58; N, 4.43. Found: C, 62.79; H, 5.20; N, 4.30.

### Example 8

### (3R)-7-[3-(4-Fluoro-phenyl)-5-(4-fluoro-phenylcarbamoyl)-1-isopropyl-4-phenyl-1H-pyrrol-2-yl]-3,5-dihydroxy-heptanoic Acid Sodium Salt

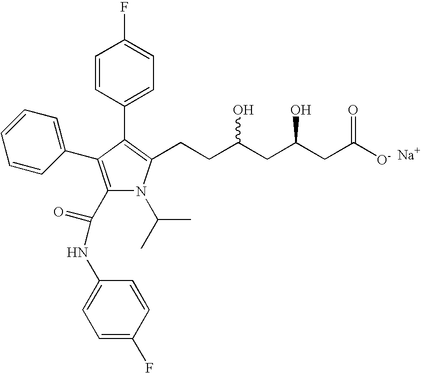 Step A

### (4R)-4-(4-Fluoro-phenyl)-5-[2-(4-hydroxy-6-oxo-tetrahydro-pyran-2-yl)-ethyl]-1-isopropyl-3-phenyl-1H-pyrrole-2-carboxylic Acid (4-fluoro-phenyl)-amide

To a solution of (3R)-3-(tert-butyl-dimethyl-silanyloxy)-7-[3-(4-fluoro-phenyl)-5-(4-fluoro-phenylcarbamoyl)-1-isopropyl-4-phenyl-1H-pyrrol-2-yl]-5-hydroxyl-heptanoic acid methyl ester prepared from Example 6, Step C (63 mg, 0.089 mmol) in acetonitrile (0.5 mL) was added dropwise a hydrogen fluoride solution (1:19 48% HF:acetonitrile, 2 mL) in an ice bath under a nitrogen atmosphere. The mixture was stirred at room temperature for 1 h. TLC showed that the reaction was complete. The mixture was partitioned between ethyl acetate and water. The organic phase was separated and washed with NaHCO₃ and brine, dried over Na₂SO₄ and filtered. The filtrate was concentrated in vacuo to give a residue, which was purified by chromatography (20%-50% ethyl acetate in hexanes) to give 29 mg (58%) of the desired product as a white foam: mp 98-99° C.; MS(APCI⁺): m/z 559.2 (MH⁺); Anal. Calcd for C₃₃H₃₂F₂N₂O₄.0.25EtOAc: C, 70.33; H, 5.90; N, 4.82. Found: C, 70.21; H, 6.26; N, 4.63.

Step B

### (3R)-7-[3-(4-Fluoro-phenyl)-5-(4-fluoro-phenylcarbamoyl)-1-isopropyl-4-phenyl-1H-pyrrol-2-yl]-3,5-dihydroxy-heptanoic Acid Sodium Salt

To a mixture of (4R)-4-(4-fluoro-phenyl)-5-[2-(4-hydroxy-6-oxo-tetrahydro-pyran-2-yl)-ethyl]-1-isopropyl-3-phenyl-1H-pyrrole-2-carboxylic acid (4-fluoro-phenyl)-amide prepared from step A (22.8 mg, 0.041 mmol) in a solution of absolute ethanol (0.5 mL) and water (0.5 mL) was added 1N aqueous sodium hydroxide solution (0.041 mL) at room temperature. The mixture was stirred for 1 h and then concentrated in vacuo to give a residue, which was dissolved in a solution of 20% methanol in methylene chloride and filtered. The filtrate was concentrated in vacuo to give a solid. The solid was triturated with diethyl ether and filtered and dried in vacuo to give 24 mg (98%) of the desired product as a white solid: mp 240-242° C.; MS(APCI⁻): m/z 575.3 (M−H); Anal. Calcd for C₃₃H₃₃F₂N₂O₅Na₁.3.65H₂O.0.75CH₂Cl₂: C, 55.68; H, 5.79; N, 3.85. Found: C, 55.32; H, 5.40; N, 3.46.

### Example 9

### (3R,5R)-7-[5-(4-Fluoro-benzylcarbamoyl)-3-(4-fluoro-phenyl)-1-isopropyl-4-phenyl-1H-pyrrol-2-yl]-3,5-dihydroxy-heptanoic Acid Sodium Salt

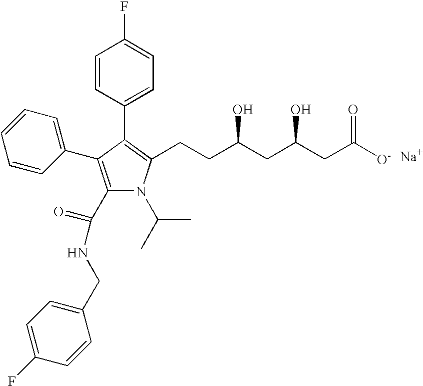 Step A

### 4-(4-Fluoro-phenyl)-5-formyl-1-isopropyl-3-phenyl-1H-pyrrole-2-carboxylic Acid 4-fluoro-benzylamide

A mixture of 4-(4-fluoro-phenyl)-5-formyl-1-isopropyl-3-phenyl-1H-pyrrole-2-carboxylic acid prepared from Example 1, Step G (0.7 g, 2.0 mmol) in thionyl chloride (5 mL) was heated at reflux for 1 h. The resulting mixture was concentrated in vacuo to give a residue, which was dried in vacuo for 1 h. The crude acid chloride was dissolved in THF (10 mL) under a nitrogen atmosphere. The mixture was cooled in an ice bath and 4-fluoro-benzylamine (0.30 mL, 2.59 mmol) was added followed by triethylamine (0.56 mL, 3.98 mmol). The mixture was stirred at room temperature overnight and partitioned between ethyl acetate and water. The organic phase was separated and washed with 1N HCl, NaHCO₃ and brine, dried over Na₂SO₄ and filtered. The filtrate was concentrated in vacuo to give a residue, which was purified by chromatography (2%-18% ethyl acetate in hexanes) to give 0.71 g (77%) of the desired product as a light yellow solid: mp 171-172° C.; MS(APCI⁻): m/z 457.2 (M−H); Anal. Calcd for C₂₈H₂₄F₂N₂O₂: C, 73.35; H, 5.28; N, 6.11. Found: C, 73.16; H, 5.27; N, 6.00.

Step B

### (3R)-3-(tert-Butyl-dimethyl-silanyloxy)-7-[5-(4-fluoro-benzylcarbamoyl)-3-(4-fluoro-phenyl)-1-isopropyl-4-phenyl-1H-pyrrol-2-yl]-5-oxo-hept-6-enoic Acid Methyl Ester

To a mixture of 4-(4-fluoro-phenyl)-5-formyl-1-isopropyl-3-phenyl-1H-pyrrole-2-carboxylic acid 4-fluoro-benzylamide prepared from step A (0.50 g, 1.09 mmol) in toluene (20 mL) at room temperature under a nitrogen atmosphere was added wittig reagent [3-(tert-butyl-dimethyl-silanyloxy)-5-oxo-6-(triphenyl-phosphanylidene)-hexanoic acid methyl ester] (0.87 g, 1.64 mmol). The mixture was heated at reflux for 42 h and then concentrated in vacuo to give a residue, which was purified by chromatography (2%-20% ethyl acetate in hexanes) to give 0.5 g (64%) of the desired product as a light yellow foam: mp 62-63° C.; MS(APCI⁺): m/z 715.3 (MH⁺); Anal. Calcd for C₄₁H₄₈F₂N₂O₅Si₁: C, 68.88; H, 6.77; N, 3.92. Found: C, 68.76; H, 6.80; N, 3.78.

Step C

### (3R)-7-[5-(4-Fluoro-benzylcarbamoyl)-3-(4-fluoro-phenyl)-1-isopropyl-4-phenyl-1H-pyrrol-2-yl]-3-hydroxy-5-oxo-hept-6-enoic Acid Methyl Ester

To a solution of (3R)-3-(tert-butyl-dimethyl-silanyloxy)-7-[5-(4-fluoro-benzylcarbamoyl)-3-(4-fluoro-phenyl)-1-isopropyl-4-phenyl-1H-pyrrol-2-yl]-5-oxo-hept-6-enoic acid methyl ester prepared from step B (550 mg, 0.77 mmol) in acetonitrile (1 mL) was added dropwise a hydrogen fluoride solution (1:19 48% HF:acetonitrile, 4 mL) in an ice bath under a nitrogen atmosphere. The mixture was stirred at room temperature for 1 h. TLC showed that the reaction was complete. The mixture was partitioned between ethyl acetate and water. The organic phase was separated and washed with NaHCO₃ and brine, dried over Na₂SO₄ and filtered. The filtrate was concentrated in vacuo to give 462 mg (100%) of the desired product as a light yellow foam: mp 62-63° C.; MS(APCI⁺): m/z 601.2 (MH⁺); Anal. Calcd for C₃₅H₃₄F₂N₂O₅.0.50H₂O: C, 68.95; H, 5.79; N, 4.59. Found: C, 68.88; H, 5.42; N, 4.44.

Step D

### (3R,5S)-7-[5-(4-Fluoro-benzylcarbamoyl)-3-(4-fluoro-phenyl)-1-isopropyl-4-phenyl-1H-pyrrol-2-yl]-3,5-dihydroxy-hept-6-enoic Acid Methyl Ester

To a mixture of (3R)-7-[5-(4-Fluoro-benzylcarbamoyl)-3-(4-fluoro-phenyl)-1-isopropyl-4-phenyl-1H-pyrrol-2-yl]-3-hydroxy-5-oxo-hept-6-enoic acid methyl ester prepared from step C (459 mg, 0.76 mmol) in THF (8 mL) and methanol (2 mL) was added dropwise a solution of 1M diethyl-methoxy-borane in THF (0.76 mL) at −78° C. under a nitrogen atmosphere. The mixture was stirred for 0.5 h and then sodium borohydride (29 mg, 0.76 mmol) was added in portions. After stirring for 2 h, 2 drops of acetic acid was added. The mixture was partitioned between ethyl acetate and water. The organic phase was separated and washed with NaHCO₃ and brine, dried over Na₂SO₄ and filtered. The filtrate was concentrated in vacuo to give a residue, which was dissolved in warm methanol and concentrated in vacuo again to give a residue, which was purified by chromatography (20%-75% ethyl acetate in hexanes) to give 370 mg (80%) of the desired product as an off-white solid: mp 68-69° C.; MS(APCI⁺): m/z 603.2, (MH⁺); Anal. Calcd for C₃₅H₃₆F₂N₂O₅.0.2EtOAc: C, 69.08; H, 6.16; N, 4.55. Found: C, 68.82; H, 6.03; N, 4.52.

Step E

### (3R,5R)-7-[5-(4-Fluoro-benzylcarbamoyl)-3-(4-fluoro-phenyl)-1-isopropyl-4-phenyl-1H-pyrrol-2-yl]-3,5-dihydroxy-heptanoic Acid Methyl Ester

To a solution of (3R,5R)-7-[5-(4-fluoro-benzylcarbamoyl)-3-(4-fluoro-phenyl)-1-isopropyl-4-phenyl-1H-pyrrol-2-yl]-3,5-dihydroxy-hept-6-enoic acid methyl ester prepared from step D (234 mg, 0.39 mmol) in ethanol (20 mL) was added 10% palladium on activated carbon (40 mg). The mixture was stirred at room temperature under a hydrogen atmosphere for 3 h. TLC showed that the reaction was complete. The mixture was filtered through celite and washed with ethyl acetate. The filtrate was concentrated in vacuo to give 234 mg (100%) white solid: mp 49-50° C.; MS(APCI⁺): m/z 605.3 (MH⁺); Anal. Calcd for C₃₅H₃₈F₂N₂O₇.0.3EtOAc: C, 68.89; H, 6.45; N, 4.44. Found: C, 68.53; H, 6.29; N, 4.54.

Step F

### (3R,5R)-7-[5-(4-Fluoro-benzylcarbamoyl)-3-(4-fluoro-phenyl)-1-isopropyl-4-phenyl-1H-pyrrol-2-yl]-3,5-dihydroxy-heptanoic Acid Sodium Salt

To a mixture of (3R,5R)-7-[5-(4-fluoro-benzylcarbamoyl)-3-(4-fluoro-phenyl)-1-isopropyl-4-phenyl-1H-pyrrol-2-yl]-3,5-dihydroxy-heptanoic acid methyl ester prepared from step E (223 mg, 0.37 mmol) in a solution of absolute ethanol (2 mL) and water (1 mL) was added 1N aqueous sodium hydroxide solution (0.37 mL) at room temperature. The mixture was stirred for 1 h and then concentrated in vacuo to give a residue, which was dissolved in a solution of 20% methanol in methylene chloride and filtered. The filtrate was concentrated in vacuo to give a solid. The solid was triturated with diethyl ether and filtered and dried in vacuo to give 220 mg (97%) of the desired product as a white solid: mp 218-220° C.; MS(APCI⁻): m/z 589.3 (M−H); Anal. Calcd for C₃₄H₃₅F₂N₂O₅Na₁.1.5H₂O: C, 63.84; H, 5.99; N, 4.38. Found: C, 63.74; H, 5.86; N, 4.10.

### Example 10

### (3R,5S)-7-[5-(4-Fluoro-benzylcarbamoyl)-3-(4-fluoro-phenyl)-1-isopropyl-4-phenyl-1H-pyrrol-2-yl]-3,5-dihydroxy-hept-6-enoic Acid Sodium Salt

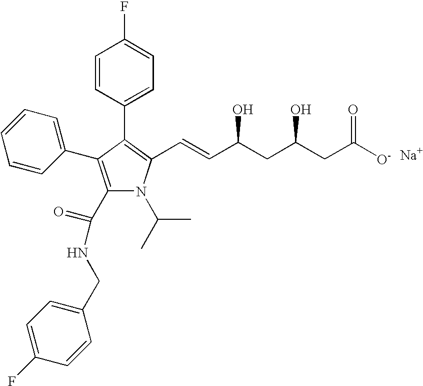

To a mixture of (3R,5R)-7-[5-(4-fluoro-benzylcarbamoyl)-3-(4-fluoro-phenyl)-1-isopropyl-4-phenyl-1H-pyrrol-2-yl]-3,5-dihydroxy-hept-6-enoic acid methyl ester prepared from Example 9, Step D (61.9 mg, 0.103 mmol) in a solution of absolute ethanol (1 mL) and water (0.5 mL) was added 1N aqueous sodium hydroxide solution (0.103 mL) at room temperature. The mixture was stirred for 1 h and then concentrated in vacuo to give a residue, which was dissolved in a solution of 20% methanol in methylene chloride and filtered. The filtrate was concentrated in vacuo to give a solid. The solid was triturated with diethyl ether and filtered and dried in vacuo to give 62 mg (99%) of the desired product as a light yellow solid: mp 221-223° C.; MS(APCI⁻): m/z 588.3 (M−H); Anal. Calcd for C₃₄H₃₃F₂N₂O₅Na₁.1.5H₂O: C, 64.04; H, 5.69; N, 4.38. Found: C, 63.67; H, 5.50; N, 4.24.

Following a similar method as described in Examples 9 and 10, the following final products were made as shown in Tables I and II.

TABLE I
Saturated Final Products.

| # | Structure | Name | MS | HPLC |
|---|---|---|---|---|
| I-1 | 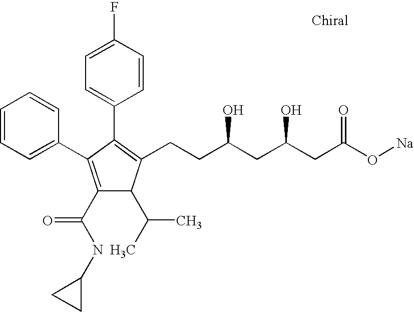 | 7-[5- Cyclopropylcarbamoyl -3-(4-fluoro-phenyl)-1- isopropyl-4-phenyl- 1H- pyrrol-2-yl]-3,5- dihydroxy-heptanoic acid, sodium salt | 523 | HPLC-96% t_{R} = 11.70 mins. |
| I-2 | 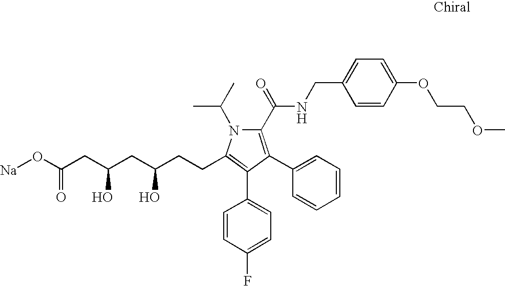 | 7-{3-(4-Fluoro- phenyl)-1-isopropyl-5- [4-(2-methoxy- ethoxy)-benzylcarba moyl]-4-phenyl-1H- pyrrol-2-yl}-3,5- dihydroxy-heptanoic acid, sodium salt | 647 | HPLC-99% t_{R} = 12.60 mins. |
| I-3 | 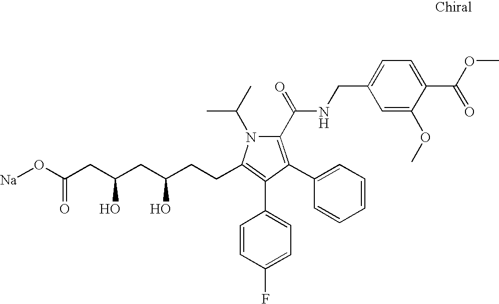 | 4-({[5-(6-Carboxy-3,5- dihydroxy-hexyl)-4-(4- fluoro-phenyl)-1- isopropyl -3-phenyl-1H-pyrrole- 2-carbonyl]-amino}- methyl)-2-methoxy- benzoic acid methyl ester, sodium salt. | 661 | HPLC-94% t_{R} = 14.71 mins. |
| I-4 | 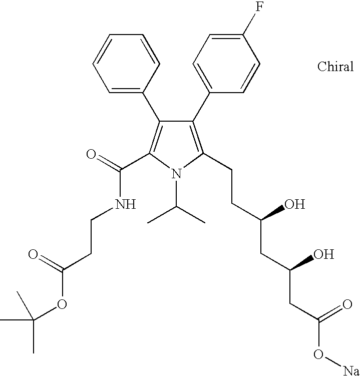 | 7-[5-(2- tertButoxycarbonyl- ethylcarbamoyl)-3-(4- fluoro-phenyl)-1-isop ropyl-4-phenyl-1H- pyrrol-2-yl]-3,5- dihydroxy-heptanoic acid, sodium salt | 611 | HPLC-97% t_{R} = 2.48 mins. |
| I-5 | 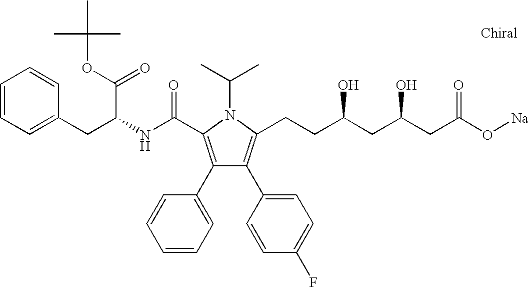 | 7-[5-(1- tertButoxycarbonyl-2- phenyl- ethylcarbamoyl)-3-(4- fluoro-phenyl)-1- isopropyl-4-phenyl- 1H-pyrrol-2-yl]-3,5- dihydroxy-heptanoic acid, sodium salt | 687 | HPLC-99% t_{R} = 18.28 mins. |
| I-6 | 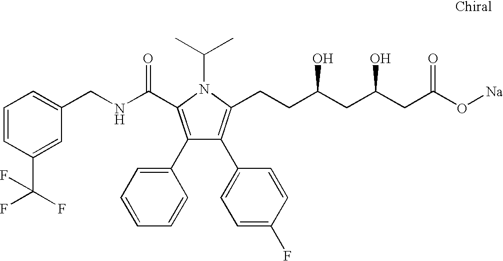 | 7-[3-(4-Fluoro- phenyl)-1-isopropyl-4- phenyl-5-(3- trifluoromethyl- benzylcarbamoyl)-1H- pyrrol-2-yl]-3,5- dihydroxy-heptanoic acid, sodium salt | 641 | HPLC-99% t_{R} = 17.14 mins. |
| I-7 | 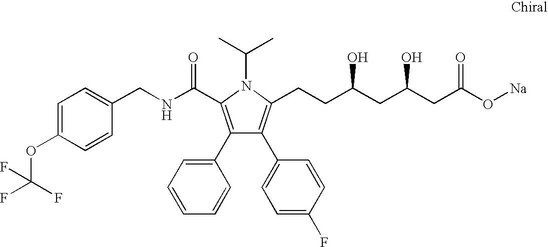 | 7-[3-(4-Fluoro- phenyl)-1-isopropyl-4- phenyl-5-(4- trifluoromethoxy- benzylcarbamoyl)-1H- pyrrol-2-yl]-3,5- dihydroxy-heptanoic acid, sodium salt | 657 | HPLC-98% t_{R} = 17.25 mins. |
| I-8 | 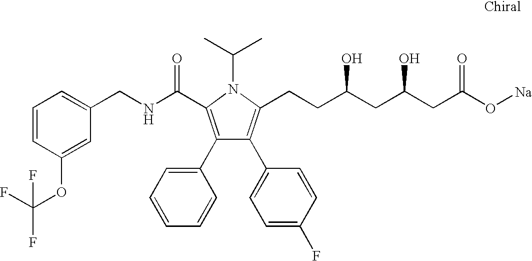 | 7-[3-(4-Fluoro- phenyl)-1-isopropyl-4- phenyl-5-(3- trifluoromethoxy- pyrrol-2-yl]-3,5- dihydroxy-heptanoic acid, sodium salt | 657 | HPLC-95% t_{R} = 17.24 mins. |
| I-9 | 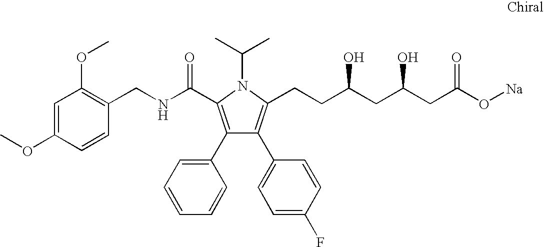 | 7-[5-(2,4-Dimethoxy- benzylcarbamoyl)-3- (4-fluoro-phenyl)-1- isopropyl-4-phenyl- 1H-pyrrol-2-yl]-3,5- dihydroxy-heptanoic acid, sodium salt | 633 | HPLC-98% t_{R} = 15.83 mins. |
| I-10 | 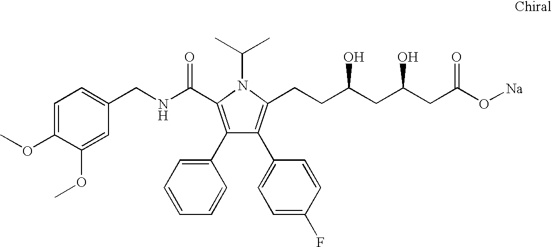 | 7-[5-(3,4-Dimethoxy- benzylcarbamoyl)-3- (4-fluoro-phenyl)-1- isopropyl-4-phenyl- 1H-pyrrol-2-yl]-3,5- dihydroxy-heptanoic acid | 633 | HPLC-97% t_{R} = 14.55 mins. |
| I-11 | 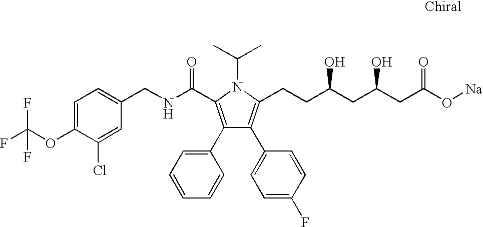 | 7-[5-(3-Chloro-4- trifluoromethoxy benzylcarbamoyl)-3- (4-fluoro-phenyl)-1- isopropyl-4-phenyl- 1H-pyrrol-2-yl]-3,5- dihydroxy-heptanoic acid, sodium salt | 691 | HPLC-72% t_{R} = 15.00 mins. |
| I-12 | 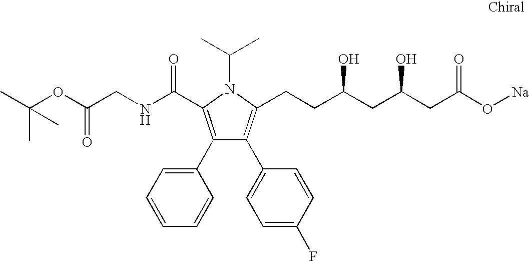 | 7-[5-(tert- Butoxycarbonylmethyl -carbamoyl)-3-(4- isopropyl-4-phenyl- 1H-pyrrol-2-yl]-3,5- dihydroxy-heptanoic acid, sodium salt | 597 | HPLC-92% t_{R} = 13.05 mins. |
| I-13 | 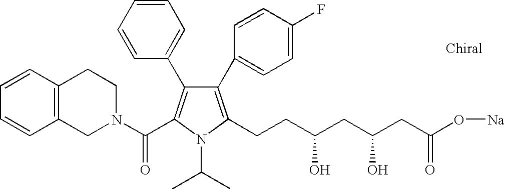 | 7-[5-(3,4-Dihydro-1H- isoquinoline-2- carbonyl)-3-(4-fluoro- phenyl)-1-isopropyl-4- phenyl-1H-pyrrol-2- yl]-3,5-dihydroxy- heptanoic acid, sodium salt | 621 | HPLC t_{R} = 16.64 min (98% pure) |
| I-14 | 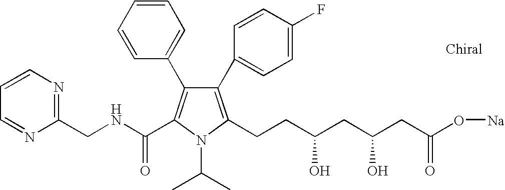 | 7-{3-(4-Fluoro- phenyl)-1-isopropyl-4- phenyl-5-[(pyrimidin- 2-ylmethyl)- carbamoyl]-1H-pyrrol- 2-yl }-3,5-dihydroxy- heptanoic acid, sodium salt | 575 | HPLC t_{R} = 10.76 min (88% pure) |
| I-15 | 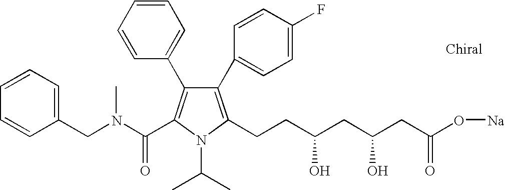 | 7-[5-(Benzyl-methyl- carbamoyl)-3-(4- fluoro-phenyl)-1- isopropyl-4-phenyl- 1H-pyrrol-2-yl]-3,5- dihydroxy-heptanoic acid, sodium salt | 587 | HPLC t_{R} = 16.47 min (97% pure) |
| I-16 | 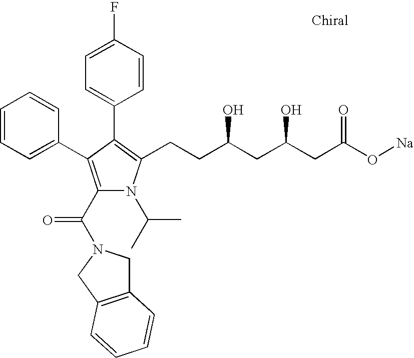 | 7-[5-(1,3-Dihydro- isoindole-2-carbonyl)- 3-(4-fluoro-phenyl)-1- isopropyl-4-phenyl- 1H-pyrrol-2-yl]-3,5- dihydroxy-heptanoic acid, sodium salt | 585 | HPLC t_{R} = 15.85 min (96% pure) |
| I-17 | 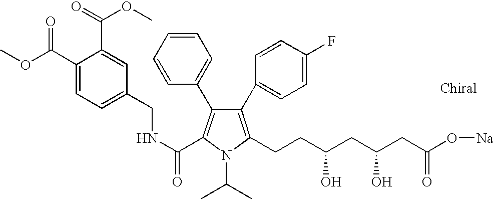 | 4-({[5-(6-Carboxy-3,5- dihydroxy-hexyl)-4-(4- fluoro-phenyl)-1- isopropyl-3-phenyl- 1H-pyrrole-2- carbonyl]-amino}- methyl)-phthalic acid dimethyl ester, sodium salt | 689 | HPLC t_{R} = 14.98 min (90% pure) |
| I-18 | 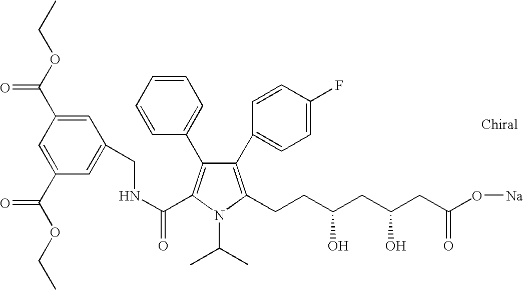 | 5-({[5-(6-Carboxy-3,5- dihydroxy-hexyl)-4-(4- fluoro-phenyl)-1- isopropyl-3-phenyl- 1H-pyrrole-2- carbonyl]-amino}- methyl)-isophthalic acid diethyl ester, sodium salt | 717 | HPLC t_{R} = 17.01 min (92% pure) |
| I-19 | 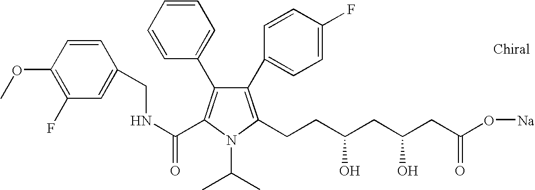 | 7-[5-(3-Fluoro-4- methoxy- benzylcarbamoyl)-3- (4-fluoro-phenyl)-1- isopropyl-4-phenyl- 1H-pyrrol-2-yl]-3,5- dihydroxy-heptanoic acid, sodium salt | 621 | HPLC t_{R} = 15.62 min (94% pure) |
| I-20 | 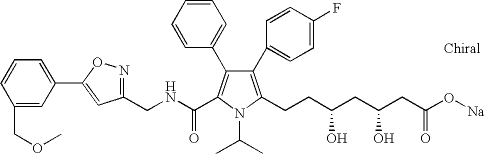 | 7-(3-(4-Fluoro- phenyl)-1-isopropyl-5- {[5-(3- methoxymethyl- phenyl)-isoxazol-3- ylmethyl]-carbamoyl}- 4-phenyl-1H-pyrrol-2- yl)-3,5-dihydroxy- heptanoic acid, sodium salt | 684 | HPLC t_{R} = 10.81 min (88% pure) |
| I-21 | 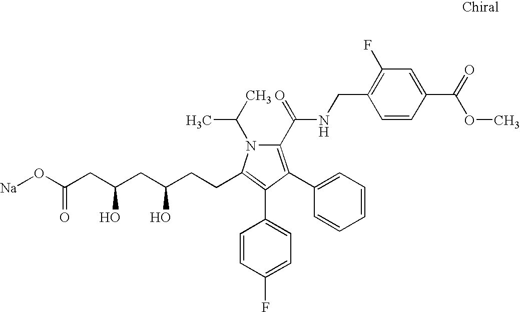 | 4-({[5-(6-Carboxy-3,5- dihydroxy-hexyl)-4-(4- fluoro-phenyl)-1- isopropyl -3-phenyl-1H-pyrrole- 2-carbonyl]-amino}- methyl)-3-fluoro- benzoic acid methyl ester, sodium salt | 649 | HPLC-96% t_{R} = 15.87 mins. |
| I-22 | 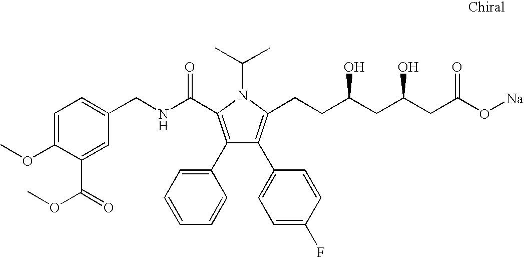 | 5-({[5-(6-Carboxy-3,5- dihydroxy-hexyl)-4-(4- fluoro-phenyl)-1- isopropyl-3-phenyl- 1H-pyrrole-2- carbonyl]-amino}- methyl)-2-methoxy- benzoic acid methyl ester, sodium salt | 661 | HPLC-95% t_{R} = 12.12 mins. |
| I-23 | 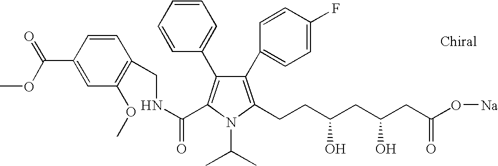 | 4-({[5-(6-Carboxy-3,5- dihydroxy-hexyl)-4-(4- fluoro-phenyl)-1- isopropyl-3-phenyl- 1H-pyrrole-2- carbonyl]-amino}- methyl)-3-methoxy- benzoic acid methyl ester, sodium salt | 660 | HPLC t_{R} = 15.74 min (90% pure) |
| I-24 | 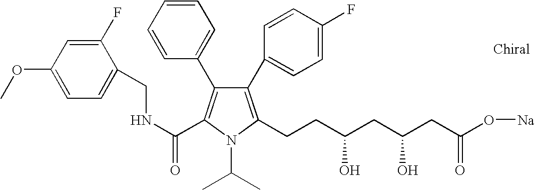 | 7-[5-(2-Fluoro-4- methoxy- benzylcarbamoyl)-3- (4-fluoro-phenyl)-1- isopropyl-4-phenyl- 1H-pyrrol-2-yl]-3,5- dihydroxy-heptanoic acid, sodium salt | 621 | HPLC t_{R} = 15.92 min (93% pure) |
| I-25 | 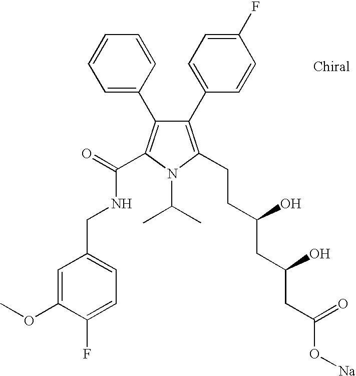 | 7-[5-(4-Fluoro-3- methoxy- benzylcarbamoyl)-3- (4-fluoro-phenyl)-1- isopropyl-4-phenyl-1H- pyrrol-2-yl]-3,5- dihydroxy-heptanoic acid, sodium salt | 621 | HPLC-98% t_{R} = 15.81 mins. |

TABLE II
Unsaturated Final Products.

| # | Structure | Name | MS | HPLC |
|---|---|---|---|---|
| II-1 | 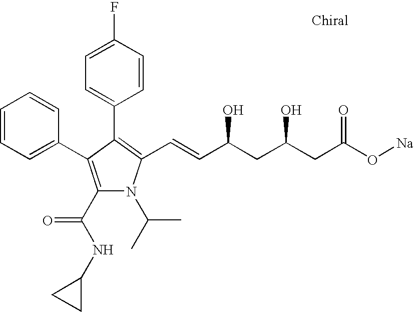 | 7-[5- Cyclopropylcarbamoyl- 3-(4-fluoro-phenyl)-1- isopropyl-4-phenyl-1H- pyrrol-2-yl]-3,5- dihydroxy-hept-6-enoic acid, sodium salt | 521 | HPLC-95% t_{R} = 11.40 mins. |
| II-2 | 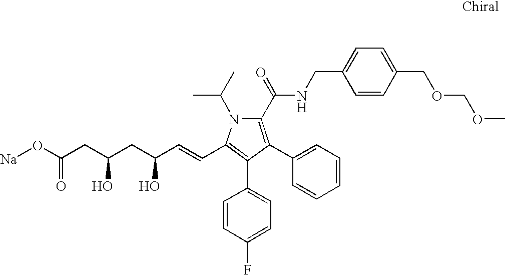 | 7-{3-(4-Fluoro-phenyl)- 1-isopropyl-5-[4-(2- methoxy-ethoxy)- benzylcarbamoyl]-4- phenyl- H-pyrrol-2-yl}- 3,5- dihydroxy-hept-6-enoic acid, sodium salt | 645 | HPLC-98% t_{R} = 12.27 mins. |
| II-3 | 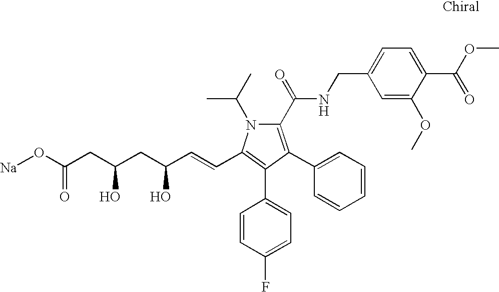 | 4-({[5-(6-Carboxy-3,5- (dihydroxy-hex-1-enyl)- 4-(4-fluoro-phenyl)-1- isopropyl-3-phenyl-1H- pyrrole-2-carbonyl]- amino}-methyl)-2- methoxy-benzoic acid methyl ester, sodium salt | 658 | HPLC-99% t_{R} = 13.00 mins. |
| II-4 | 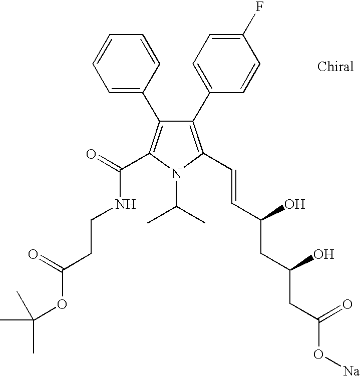 | 7-[5(2-tert- Butoxycarbonyl- ethylcarbamoyl)-3-(4- fluoro-phenyl)-1- isopropyl-4-phenyl-1H- pyrrol-2-yl]-3,5- dihydroxy-hept-6-enoic acid, sodium salt | 609 | HPLC-98% t_{R} = 12.85 mins. |
| II-5 | 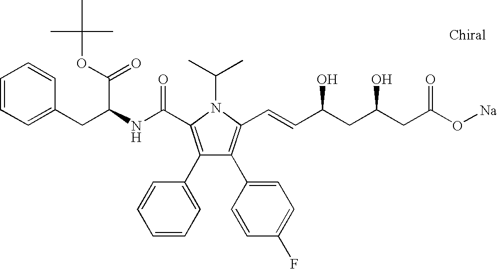 | 7-[5-(1-tert- Butoxycarbonyl-2- phenyl-ethylcarbamoyl)- 3-(4-fluoro-phenyl)-1- isopropyl-4-phenyl-1H- pyrrol-2-yl]-3,5- dihydroxy-hept-6-enoic acid, sodium salt | 685 | HPLC-96% t_{R} = 17.88 mins. |
| II-6 | 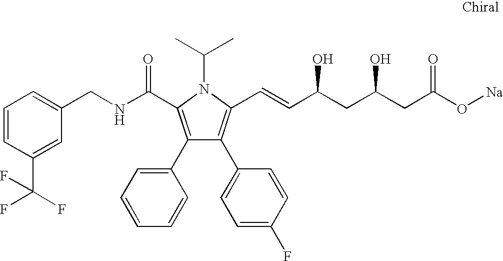 | 7-[3-(4-Fluoro-phenyl)- 1-isopropyl-4-phenyl-5- (3-trifluoromethyl- benzyicarbamoyl)-1H- dihydroxy-hept-6-enoic acid, sodium salt | 661 | HPLC-94% t_{R} = 16.64 mins. |
| II-7 | 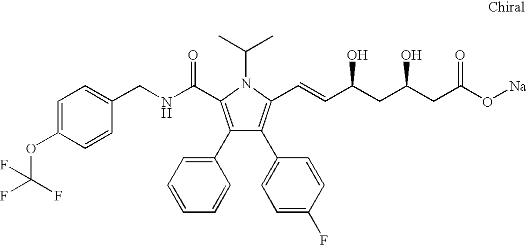 | 7-[3-(4-Fluoro-phenyl)- 1-isopropyl-4-phenyl-5- (4-trifluoromethoxy- pyrrol-2-yl]-3,5- dihydroxy-hept-6-enoic acid, sodium salt | 655 | HPLC-96% t_{R} = 16.83 mins. |
| II-8 | 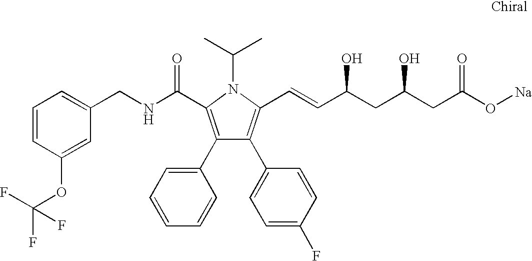 | 7-[3-(4-Fluoro-phenyl)- 1-isopropyl-4-phenyl-5- (3-trifluoromethoxy- benzylcarbamoyl)-1H- pyrrol-2-yl]-3,5- dihydroxy-hept-6-enoic acid, sodium salt | 655 | HPLC-91% t_{R} = 16.88 mins. |
| II-9 | 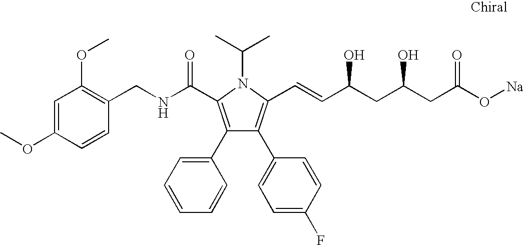 | 7-[5-(2,4-Dimethoxy- benzylcarbamoyl)-3-(4- fluoro-phenyl)-1- isopropyl-4-phenyl-1H- pyrrol-2-yl]-3,5- dihydroxy-hept-6-enoic acid, sodium salt | 631 | HPLC-92% t_{R} = 15.46 mins. |
| II-10 | 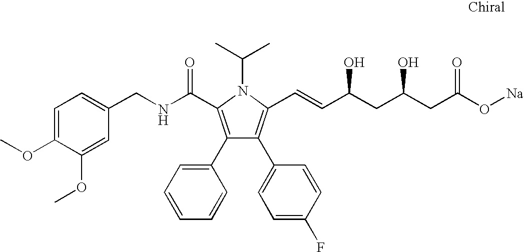 | 7-[5-(3,4-Dimethoxy- benzylcarbamoyl)-3-(4- fluoro-phenyl)-1- isopropyl-4-phenyl-1H- pyrrol-2-yl]-3,5- dihydroxy-hept-6-enoic acid, sodium salt | 631 | HPLC-92% t_{R} = 14.14 mins. |
| II-11 | 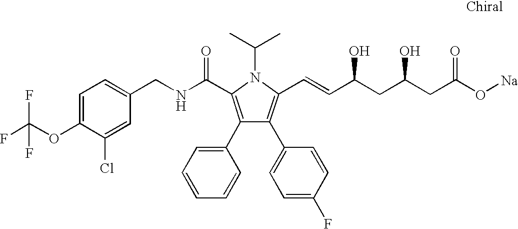 | 7-[5-(3-Chloro-4- trifluoromethoxy- benzylcarbamoyl)-3-(4- fluoro-phenyl)-1- isopropyl-4-phenyl-1H- pyrrol-2-yl]-3,5- dihydroxy-hept-6-enoic acid, sodium salt | 690 | HPLC-92% t_{R} = 17.59 mins. |
| II-12 | 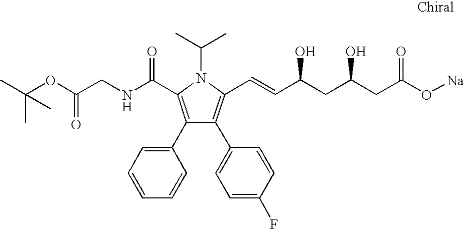 | 7-[5-(tert- Butoxycarbonylmethyl carbamoyl)-3-(4-fluoro- phenyl)-1-isopropyl-4- phenyl-1H-pyrrol-2-yl]- 3,5-dihydroxy-hept-6- enoic acid, sodium salt | 595 | HPLC-87% t_{R} = 12.82 mins. |
| II-13 | 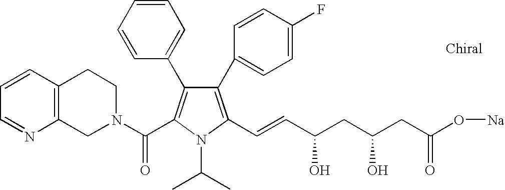 | 7-[5-(3,4-Dihydro-1H- isoquinoline-2- carbonyl)-3-(4-fluoro- phenyl)-1-isopropyl-4- phenyl-1H-pyrrol-2-yl]- 3,5-dihydroxy-hept-6- enoic acid, sodium salt | 619 | HPLC t_{R} = 16.16 min (92% pure) |
| II-14 | 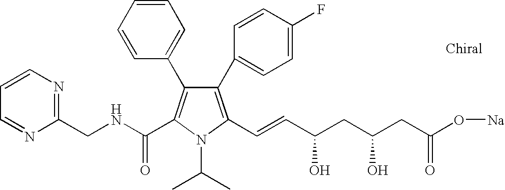 | 7-{3-(4-Fluoro-phenyl)- 1-isopropyl-4-phenyl-5- [(pyrimidin-2-ylmethyl)- carbamoyl]-1H-pyrrol-2- yl}-3,5-dihydroxy-hept- 6-enoic acid, sodium salt | 573 | HPLC t_{R} = 12.19 min (93% pure) |
| II-15 | 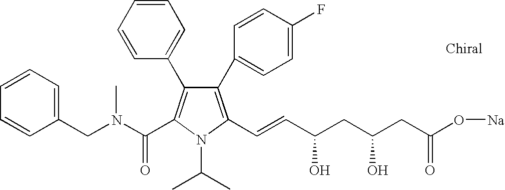 | 7-[5-(Benzyl-methyl- carbamoyl)-3-(4-fluoro- phenyl)-1-isopropyl-4- phenyl-1H-pyrrol-2-yl]- 3,5-dihydroxy-hept-6- enoic acid, sodium salt | 585 | HPLC t_{R} = 15.3 min (94% pure) |
| II-16 | 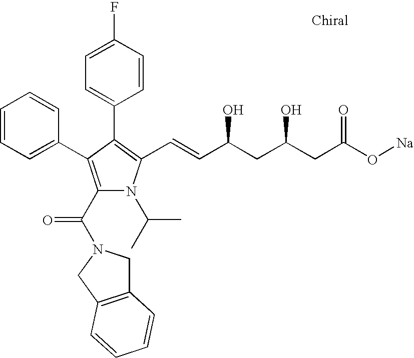 | 7-[5-(1,3-Dihydro- isoindole-2-carbonyl)-3- (4-fluoro-phenyl)-1- isopropyl-4-phenyl-1H- pyrrol-2-yl]-3,5- dihydroxy-hept-6-enoic acid, sodium salt | 583 | HPLC t_{R} = 15.49 min (93% pure) |
| II-17 | 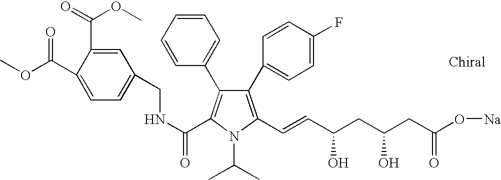 | 4-({[5-(6-Carboxy-3,5- dihydroxy-hex-1-enyl)- 4-(4-fluoro-phenyl)-1- isopropyl-3-phenyl-1H- pyrrole-2-carbonyl]- amino}-methyl)-phthalic acid dimethyl ester, sodium salt | 686 | HPLC t_{R} = 14.58 min (90% pure) |
| II-18 | 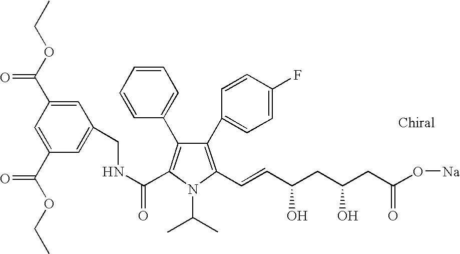 | 5-({[5-(6-Carboxy-3,5- dihydroxy-hex-1-enyl)- 4-(4-fluoro-phenyl)-1- isopropyl-3-phenyl-1H- pyrrole-2-carbonyl]- amino 1-methyl)- isophthalic acid diethyl ester, sodium salt | 715 | HPLC t_{R} = 16.65 min (92% pure) |
| II-19 | 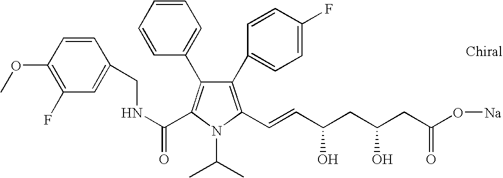 | 7-[5-(3-Fluoro-4- methoxy benzylcarbamoyl)-3-(4- fluoro-phenyl)-1- isopropyl-4-phenyl-1H pyrrol-2-yl]-3,5- dihydroxy-hept-6-enoic acid, sodium salt | 619 | HPLC t_{R} = 15.24 min (97% pure) |
| II-20 | 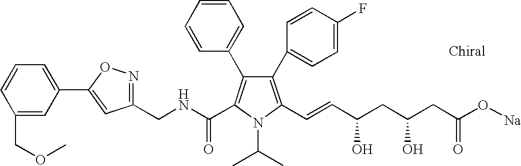 | 7-(3-(4-Fluoro-phenyl)- 1-isopropyl-5-{[5-(3- methoxymethyl-phenyl) isoxazol-3-ylmethyl]- carbamoyl}-4-phenyl- 1H-pyrrol-2-yl)-3,5- dihydroxy-hept-6-enoic acid, sodium salt | 681 | HPLC t_{R} = 15.68 min (91% pure) |
| II-21 | 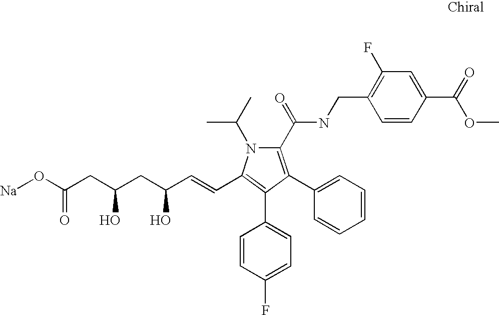 | 4-({[5-(6-Carboxy-3,5- dihydroxy-hex-1-enyl)- 4-(4-fluoro-phenyl)-1- isopropyl-3-phenyl-1H- pyrrole-2-carbonyl]- amino}-methyl)-3- fluoro-benzoic acid methyl ester, sodium salt | 645 | HPLC-95% t_{R} = 15.52 mins. |
| II-22 | 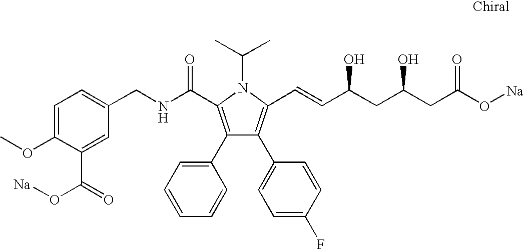 | 5-({[5-(6-Carboxy-3,5- dihydroxy-hex-1-enyl)- 4-(4-fluoro-phenyl)-1- isopropyl-3-phenyl-1H- pyrrole-2-carbonyl]- amino}-methyl)-2- methoxy-benzoic acid, disodium salt | 644 | HPLC-92% t_{R} = 11.05 mins. |
| II-23 | 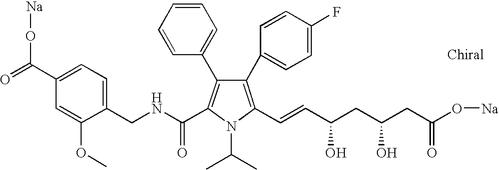 | 4-({[5-(6-Carboxy-3,5- dihydroxy-hex-1-enyl)- 4-(4-fluoro-phenyl)-1- isopropyl-3-phenyl-1H- pyrrole-2-carbonyl]- amino}-methyl)-3- methoxy-benzoic acid, disodium salt | 645 | HPLC t_{R} = 13.75 min (90% pure) |
| II- 26U | 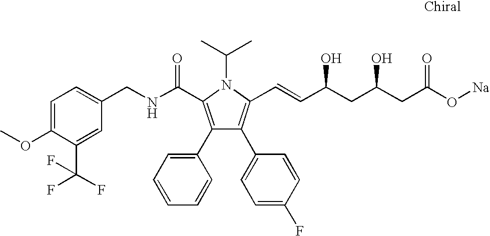 | 7-[3-(4-Fluoro-phenyl)- 1-isopropyl-5-(4- methoxy-3- trifluoromethyl- phenyl-1H-pyrrol-2-yl]- 3,5-dihydroxy-hept-6- enoic acid, sodium salt | 669 | HPLC-92% t_{R} = 16.39 mins |
| II-27 | 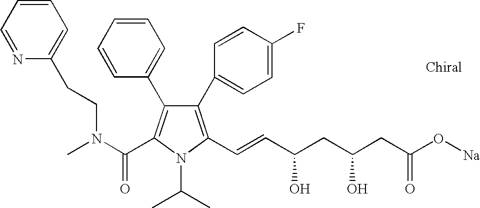 | 7-{3-(4-Fluoro-phenyl)- 1-isopropyl-5-[methyl- (2-pyridin-2-yl-ethyl)- carbamoyl]-4-phenyl- 1H-pyrrol-2-yl}-3,5- dihydroxy-hept-6-enoic acid, methoxy-benzoic acid, sodium salt | 600 | HPLC t_{R} = 10.12 min (90% pure) |
| II-28 | 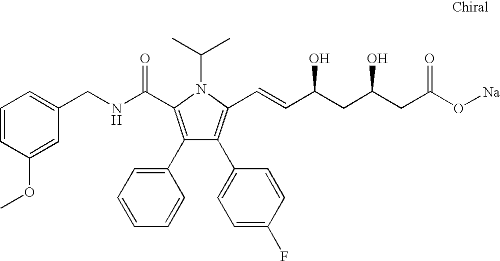 | 7-[3-(4-Fluoro-phenyl)- 1-isopropyl-5-(3- methoxy- phenyl-1H-pyrrol-2-yl]- 3,5-dihydroxy-hept-6- enoic acid, sodium salt | 601 | HPLC-93% t_{R} = 15.31 mins |

Note to Table I and II:
a. The HPLC condition, 90:10 to 10:90, 0.1% TFA water: 0.1% TFA acetonitrile, linear gradient over 20 min at 1.6 mL/min (λ = 254 nm).
b. MS-m/z (M + 1)

III. List of Intermediates for Tables I & II

| | | |
|---|---|---|
| Ex. | 1A | 1B |
| | 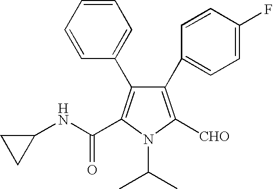 | 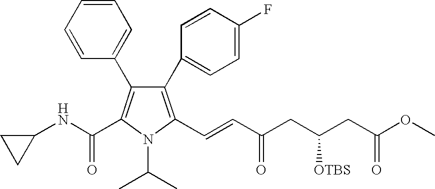 |
| | 4-(4-Fluoro- | 3-(tert-Butyl-dimethyl- |
| | phenyl)-5-formyl- | silanyloxy)-7-[5- |
| | 1-isopropyl-3- | cyclopropylcarbamoyl-3-(4- |
| | phenyl-1H- | fluoro-phenyl)-1-isopropyl- |
| | pyrrole-2- | 4-phenyl-1-pyrrol-2-yl]-5- |
| | carboxylic acid | oxo-hept-6-enoic acid |
| | cyclopropylamide | methyl ester |
| MS = | 391 | 647 |
| Ex. | 1C | 1D |
| | 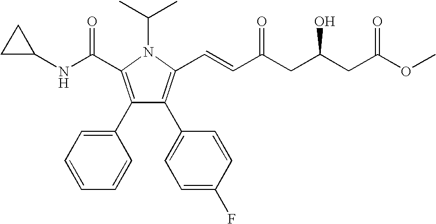 | 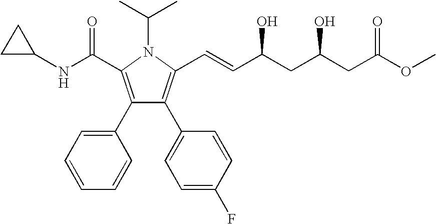 |
| | 7-[5- | 7-[5-Cyclopropylcarbamoyl- |
| | Cyclopropylcarbamoyl-3- | 3-(4-fluoro-phenyl)-1- |
| | (4-fluoro-phenyl)-1- | isopropyl-4-phenyl-1H- |
| | isopropyl-4-phenyl-1H- | pyrrol-2-yl]-3,5-dihydroxy- |
| | pyrrol-2-yl]-3-hydroxy-5- | hept-6-enoic acid methyl |
| | oxo-hept-6-enoic acid | ester |
| | methyl ester | |
| MS = | 533 | 535 |
| Ex. | 1E | 2A |
| | 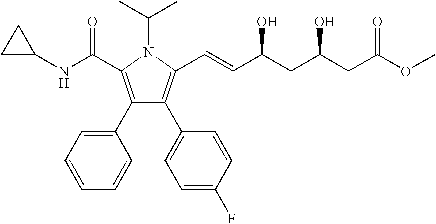 | 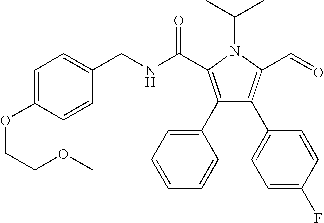 |
| | 7-[5-Cyclopropylcarbamoyl-3- | 4-(4-Fluoro- |
| | (4-fluoro-phenyl)-1-isopropyl- | phenyl)-5-formyl- |
| | 4-phenyl-1H-pyrrol-2-yl]-3,5- | 1-isopropyl-3- |
| | dihydroxy-heptanoic acid | phenyl-1H- |
| | methyl ester | pyrrole-2- |
| | | carboxylic acid 4- |
| | | (2-methoxy- |
| | | ethoxy)- |
| | | benz lamide |
| MS = | 537 | 515 |
| Ex. | 2B | 2C |
| | 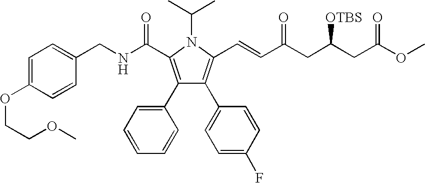 | 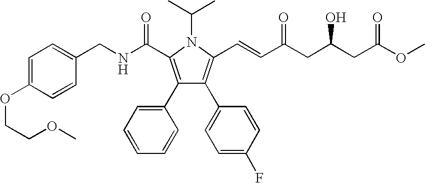 |
| | 3-(tert-Butyl-dimethyl- | 7-{3-(4-Fluoro-phenyl)-1- |
| | silanyloxy)-7-{3-(4-fluoro- | isopropyl-5-[4-(2-methoxy- |
| | phenyl)-1-isopropyl-5-[4-(2- | ethoxy)-benzylcarbamoyl]- |
| | methoxy-ethoxy)- | 4-phenyl-1H-pyrrol-2-yl}- |
| | benzylcarbamoyl]-4-phenyl- | 3-hydroxy-5-oxo-hept-6- |
| | 1H-pyrrol-2-yl}-5-oxo-hept- | enoic acid methyl ester |
| | 6-enoic acid methyl ester | |
| MS = | 771 | 657 |
| Ex. | 2D | 2E |
| | 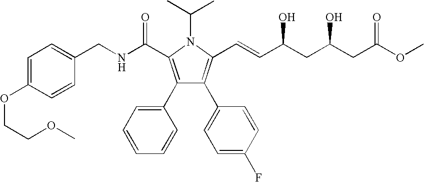 | 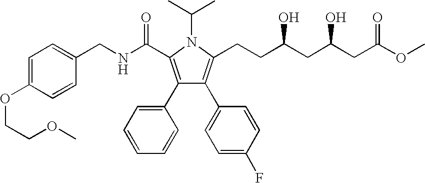 |
| | 7-{3-(4-Fluoro-phenyl)-1- | 7-{3-(4-Fluoro-phenyl)-1- |
| | isopropyl-5-[4-(2-methoxy- | isopropyl-5-[4-(2-methoxy- |
| | ethoxy)-benzylcarbamoyl]-4- | ethoxy)-benzylcarbamoyl]-4- |
| | phenyl-1H-pyrrol-2-yl}-3,5- | phenyl-1H-pyrrol-2-yl}-3,5- |
| | dihydroxy-hept-6-enoic acid | dihydroxy-heptanoic acid |
| | methyl ester | methyl ester |
| MS = | 660 | 661 |
| Ex. | 3A | 3B |
| | 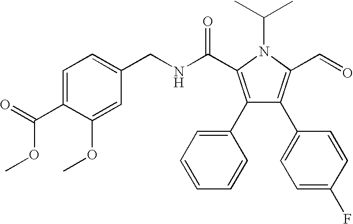 | 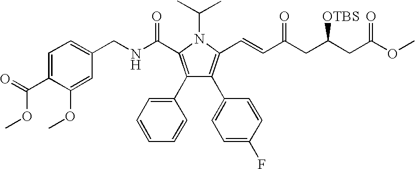 |
| | 4-(}[4-(4-Fluoro- | 4-({[5-[5-(tert-Butyl- |
| | phenyl)-5-formyl- | dimethyl-silanyloxy)-6- |
| | 1-isopropyl-3- | methoxy carbonyl-3-oxo- |
| | phenyl-1H- | hex-1-enyl]-4-(4-fluoro- |
| | pyrrole-2-carbon | phenyl)-1-isopropyl-3- |
| | yl]-amino)- | phenyl-1H-pyrrole-2-carbon |
| | methyl)-2- | yl]-amino}-methyl)-2- |
| | methoxy-benzoic | methoxy-benzoic acid |
| | acid methyl ester | methyl ester |
| MS = | 529 | 785 |
| Ex. | 3C | 3D |
| | 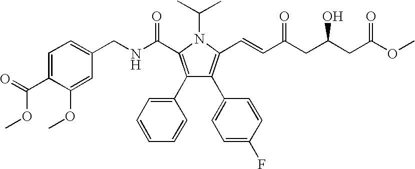 | 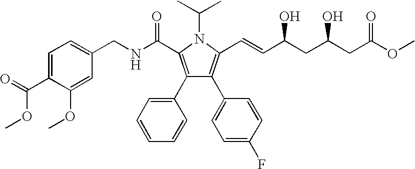 |
| | 4-({[4-(4-Fluoro-phenyl)-5- | 4-({[5-(3,5-Dihydroxy-6- |
| | (5-hydroxy-6- | methoxycarbonyl-hex-1- |
| | methoxycarbonyl-3-oxo- | enyl)-4-(4-fluoro-phenyl)-1- |
| | hex-1-enyl)-1-isopropyl-3- | isopropyl-3-phenyl-1H- |
| | phenyl-1H-pyrrole-2- | pyrrole-2-carbonyl]-amino}- |
| | carbonyl]-amino}-methyl)- | methyl)-2-methoxy-benzoic |
| | 2-methoxy-benzoic acid | acid methyl ester |
| | methyl ester | |
| MS = | 671 | 673 |
| Ex. | 3E | 4A |
| | 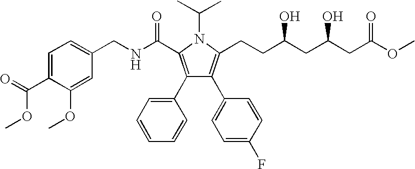 | 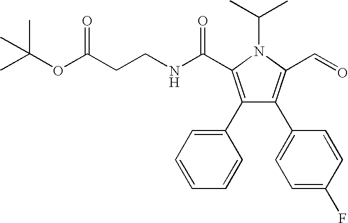 |
| | 4-({[5-(3,5-Dihydroxy-6- | 3-([4-(4-Fluoro- |
| | methoxycarbonyl-hexyl)-4-(4- | phenyl)-5-formyl- |
| | fluoro-phenyl)-1-isopropyl-3- | 1-isopropyl-3- |
| | phenyl-1H-pyrrole-2- | phenyl-1H- |
| | carbonyl]-amino}-methyl)-2- | pyrrole-2- |
| | methoxy-benzoic acid | carbonyl]-amino}- |
| | ester | propionic acid |
| | | tert-butyl ester |
| MS = | 675 | 479 |
| Ex. | 4B | 4C |
| | 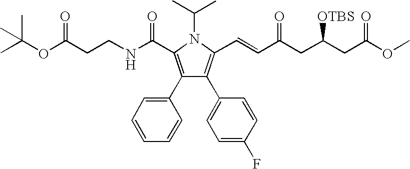 | 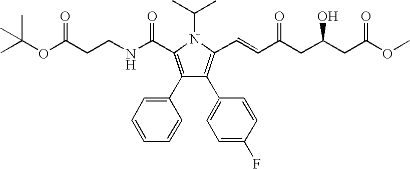 |
| | 7-[5-(2-tert- | 7-[5-(2-tert- |
| | Butoxycarbonyl- | Butoxycarbonyl- |
| | ethylcarbamoyl)-3-(4-fluoro- | ethylcarbamoyl)-3-(4- |
| | phenyl)-1-isopropyl-4- | fluoro-phenyl)-1-isopropyl- |
| | phenyl-1H-pyrrol-2-yl]-3- | 4-phenyl-1H-pyrrol-2-yl]- |
| | (tert-butyl-dimethyl- | 3-hydroxy-5-oxo-hept-6- |
| | silanyloxy)-5-oxo-hept-6- | enoic acid methyl ester |
| | enoic acid methyl ester | |
| MS = | 735 | 621 |
| Ex. | 4D | 4E |
| | 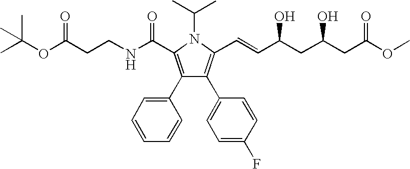 | 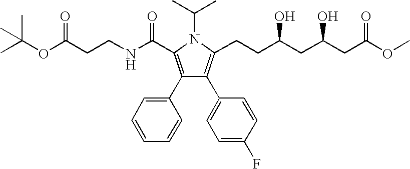 |
| | 7-[5-(2-tert-Butoxycarbonyl- | 7-[5-(2-tert-Butoxycarbonyl- |
| | ethylcarbamoyl)-3-(4-fluoro- | ethylcarbamoyl)-3-(4-fluoro- |
| | phenyl)-1-isopropyl-4- | phenyl)-1-isopropyl-4-phenyl- |
| | phenyl-1H-pyrrol-2-yl]-3,5- | 1H-pyrrol-2-yl]-3,5- |
| | dihydroxy-hept-6-enoic acid | dihydroxy-heptanoic acid |
| | methyl ester | methyl ester |
| MS = | 623 | 625 |
| Ex. | 5A | 5B |
| | 2-{[4-(4-Fluoro- | 7-[5-(1-tert-Butoxycarbonyl- |
| | phenyl)-5-formyl- | 2-phenyl-ethylcarbamoyl)-3- |
| | 1-isopropyl-3- | (4-fluoro-phenyl)-1- |
| | phenyl-1H- | isopropyl-4-phenyl-1H- |
| | pyrrole-2- | pyrrol-2-yl]-3-(tert-butyl- |
| | carbonyl]-amino}- | dimethyl-silanyloxy)-5-oxo- |
| | 3-phenyl- | hept-6-enoic acid methyl |
| | propionic | ester |
| | acid tert-butyl | |
| | ester | |
| MS = | 555 | 811 |
| Ex. | 5C | 5D |
| | 7-[5-(1-tert- | 7-[5-(1-tert-Butoxycarbonyl- |
| | Butoxycarbonyl-2-phenyl- | 2-phenyl-ethylcarbamoyl)-3- |
| | ethylcarbamoyl)-3-(4- | (4-fluoro-phenyl)-1- |
| | fluoro-phenyl)-1-isopropyl- | isopropyl-4-phenyl-1-phenyl-1H- |
| | 4-phenyl-1-pyrrol-2-yl]- | pyrrol-2-yl]-3,5-dihydroxy- |
| | 3-hydroxy-5-oxo-hept-6- | hept-6-enoic acid methyl |
| | enoic acid methyl ester | ester |
| MS = | 697 | 699 |
| Ex. | 5E | |
| | 7-[5-(1-tert-Butoxycarbonyl-2- | |
| | phenyl-ethylcarbamoyl)-3-(4- | |
| | fluoro-phenyl)-1-isopropyl-4- | |
| | phenyl-1H-pyrrol-2-yl]-3,5- | |
| | dibydroxy-heptanoic acid | |
| | methyl ester | |
| MS = | 701 | |
| Ex. | 6A | 6B |
| | 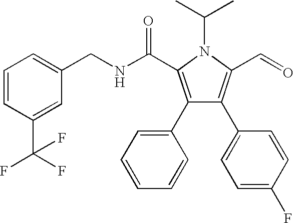 | 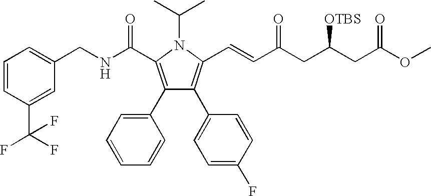 |
| | 4-(4-Fluoro- | 3-(tert-Butyl-dimethyl- |
| | phenyl)-5-formyl- | silanyloxy)-7-[3-(4-fluoro- |
| | 1-isopropyl-3- | phenyl)-1-isopropyl-4- |
| | phenyl-1H- | phenyl-5-(3-trifluoromethyl- |
| | pyrrole-2- | benzylcarbamoyl)-1H- |
| | carboxylic acid 3- | pyrrol-2-yl]-5-oxo-hept-6- |
| | trifluoromethyl- | enoic acid methyl ester |
| | benzylamide | |
| MS | m/z 509 | m/z 765 |
| | (M + 1) | (M + 1) |
| Ex. | 6C | 6D |
| | 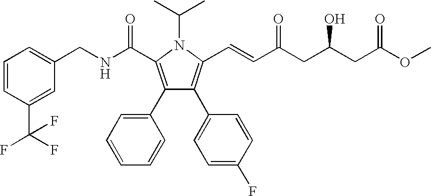 | 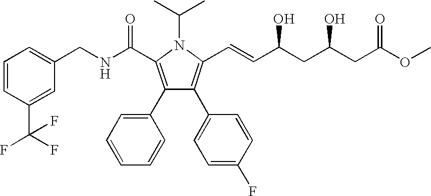 |
| | 7-[3-(4-Fluoro-phenyl)-1- | 7-[3-(4-Fluoro-phenyl)-1- |
| | isopropyl-4-phenyl-5-(3- | isopropyl-4-phenyl-5-(3- |
| | trifluoromethyl- | trifluoromethyl- |
| | benzylcarbamoyl)-1H- | benzylcarbamoyl)-1H-pyrrol- |
| | pyrrol-2-yl]-3-hydroxy-5- | 2-yl]-3,5-dihydroxy-hept-6- |
| | oxo-hept-6-enoic acid | enoic acid methyl ester |
| | methyl ester | |
| MS | m/z 651 | m/z 653 |
| | (M + 1) | (M + 1) |
| Ex. | 6E | 7A |
| | 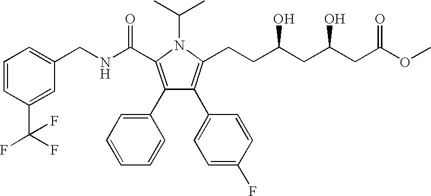 | 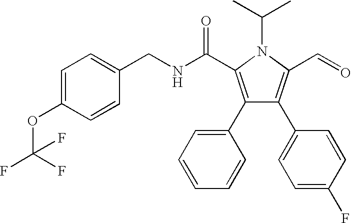 |
| | 7-[3-(4-Fluoro-phenyl)-1- | 4-(4-Fluoro- |
| | isopropyl-4-phenyl-5-(3- | phenyl)-5-formyl- |
| | trifluoromethyl- | 1-isopropyl-3- |
| | benzylcarbamoyl)-1H-pyrrol- | phenyl-1H- |
| | 2-yl]-3,5-dihydroxy-heptanoic | pyrrole-2- |
| | acid methyl ester | carboxylic acid 4- |
| | | trifluoromethoxy- |
| | | benzylamide |
| MS | m/z 655 | m/z 525 |
| | (M + 1) | (M + 1) |
| Ex. | 7B | 7C |
| | 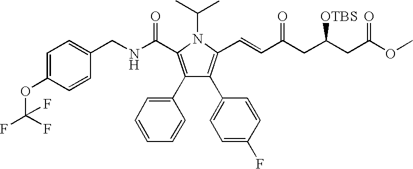 | 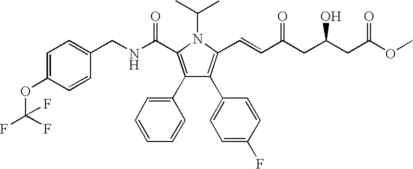 |
| | 3-(tert-Butyl-dimethyl- | 7-[3-(4-Fluoro-phenyl)-1- |
| | silanyloxy)-7-[3-(4-fluoro- | isopropyl-4-phenyl-5-(4- |
| | phenyl )-1-isopropyl-4- | tritluoromethoxy- |
| | phenyl-5-(4- | benzylcarbamoyl)-1H- |
| | trifluoromethoxy- | pyrrol-2-yl]-3-hydroxy-5- |
| | benzylcarbamoyl)-1H- | oxo-hept-6-enoic acid |
| | pyrrol-2-yl]-5-oxo-hept-6- | methyl ester |
| | enoic acid methyl ester | |
| MS | m/z 781 | m/z 667 |
| | (M + 1) | (M + 1) |
| Ex. | 7D | 7E |
| | 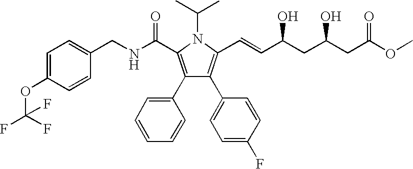 | 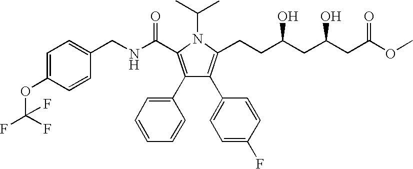 |
| | 7-[3-(4-Fluoro-phenyl)-1- | 7-[3-(4-Fluoro-phenyl)-1- |
| | isopropyl-4-phenyl-5-(4- | isopropyl-4-phenyl-5-(4- |
| | trifluoromethoxy- | trifluoromethoxy- |
| | benzylcarbamoyl)-1H-pyrrol- | benzylcarbamoyl)-1H-pyrrol- |
| | 2-yl]-3,5-dihydroxy-hept-6- | 2-yl]-3,5-dihydroxy-heptanoic |
| | enoic acid methyl ester | acid methyl ester |
| MS | m/z 669 | m/z 671 |
| | (M + 1) | (M + 1) |
| Ex. | 8A | 8B |
| | 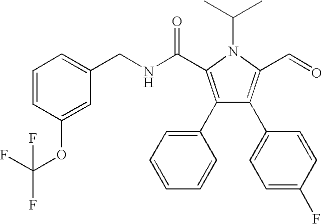 | 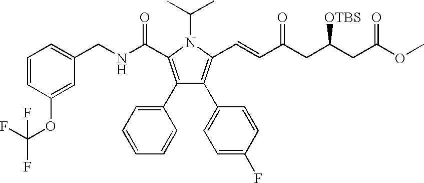 |
| | 4-(4-Fluoro- | 3-(tert-Butyl-dimethyl- |
| | phenyl)-5-formyl- | silanyloxy)-7-[3-(4-fluoro- |
| | 1-isopropyl-3- | phenyl)-1-isopropyl-4- |
| | phenyl-1H- | phenyl-5-(3- |
| | pyrrole-2- | trifluoromethoxy- |
| | carboxylic acid 3- | benzylcarbamoyl)-1H- |
| | trifluoromethoxy- | pyrrol-2-yl]-5-oxo-hept-6- |
| | benzylamide | enoic acid methyl ester |
| MS | m/z 525 | m/z 781 |
| | (M + 1) | (M + 1) |
| Ex. | 8C | 8D |
| | 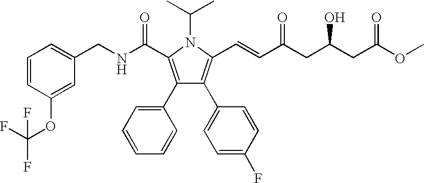 | 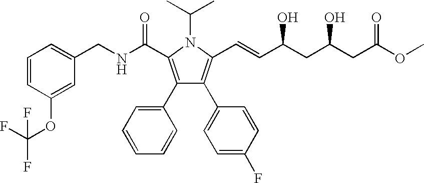 |
| | 7-[3-(4-Fluoro-phenyl)-1- | 7-[3-(4-Fluoro-phenyl)-1- |
| | isopropyl-4-phenyl-5-(3- | isopropyl-4-phenyl-5-(3- |
| | trifluoromethoxy- | trifluoromethoxy- |
| | benzylcarbamoyl)-1H- | benzylcarbamoyl)-1H-pyrrol- |
| | pyrrol-2-yl]-3-hydroxy-5- | 2-yl]-3,5-dihydroxy-hept-6- |
| | oxo-hept-6-enoic acid | enoic acid methyl ester |
| | methyl ester | |
| MS | m/z 667 | m/z 669 |
| | (M + 1) | (M + 1) |
| Ex. | 8E | 9A |
| | 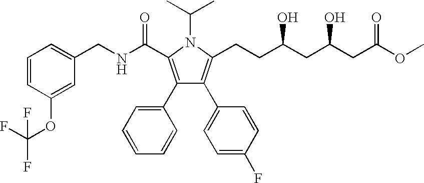 | 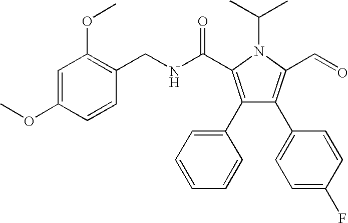 |
| | 7-113-(4-Fluoro-phenyl)-1- | 4-(4-Fluoro- |
| | isopropyl-4-phenyl-5-(3- | phenyl)-5-formyl- |
| | trifluoromethoxy- | 1-isopropyl-3- |
| | benzylcarbamoyl)-1H-pyrrol- | phenyl-1H- |
| | 2-yl]-3,5-dihydroxy-heptanoic | pyrrole-2- |
| | acid methyl ester | carboxylic acid |
| | | 2,4-dimethoxy- |
| | | benzylamide |
| MS | m/z 671 | m/z 501 |
| | (M + 1) | (M + 1) |
| Ex. | 9B | 9C |
| | 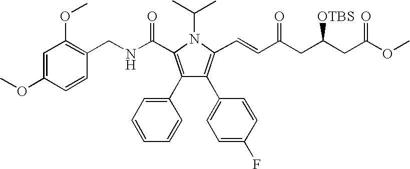 | 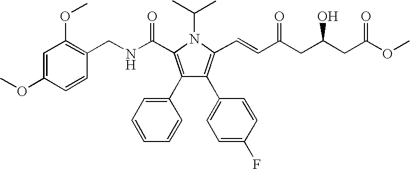 |
| | 3-(tert-Butyl-dimethyl- | 7-[5-(2,4-Dimethoxy- |
| | silanyloxy)-7-45-(2,4- | benzylcarbamoyl)-3-(4- |
| | dimethoxy- | fluoro-phenyl)-1-isopropyl- |
| | benzylcarbamoyl)-3-(4- | 4-phenyl-1-pyrrol-2-yl]- |
| | fluoro-phenyl)-1-isopropyl- | 3-hydroxy-5-oxo-hept-6- |
| | 4-phenyl-1H-pyrrol-2-yl]-5- | enoic acid methyl ester |
| | oxo-hept-6-enoic acid | |
| | methyl ester | |
| MS | m/z 757 | m/z 643 |
| | (M + 1) | (M + 1) |
| Ex. | 9D | 9E |
| | 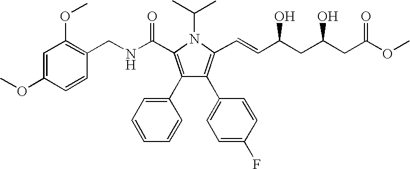 | 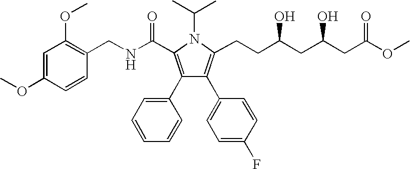 |
| | 7-[5-(2,4-Dimethoxy- | 7-[5-(2,4-Dimethoxy- |
| | benzylcarbamoyl)-3-(4- | benzylcarbamoyl)-3-(4-fluoro- |
| | fluoro-phenyl)-1-isopropyl-4- | phenyl)-1-isopropyl-4-phenyl- |
| | phenyl-1H-pyrrol-2-yl]-3,5- | 1H-pyrrol-2-yl]-3,5- |
| | dihydroxy-hept-6-enoic acid | dihydroxy-heptanoic acid |
| | methyl ester | methyl ester |
| MS | m/z 645 | m/z 647 |
| | (M + 1) | (M + 1) |
| Ex. | 10A | 10B |
| | 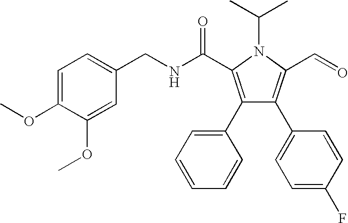 | 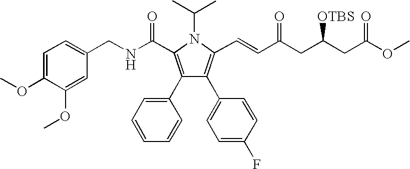 |
| | 4-(4-Fluoro- | 3-(tert-Butyl-dimethyl- |
| | phenyl)-5-formyl- | silanyloxy)-7-[5-(3,4- |
| | 1-isopropyl-3- | dimethoxy- |
| | phenyl-1H- | benzylcarbamoyl)-3-(4- |
| | pyrrole-2- | fluoro-phenyl)-1-isopropyl- |
| | carboxylic acid | 4-phenyl-1H-pyrrol-2-yl]-5- |
| | 3,4-dimethoxy- | oxo-hept-6-enoic acid |
| | benzylamide | methyl ester |
| MS | m/z 501 | m/z 747 |
| | (M + 1) | (M + 1) |
| Ex. | 10C | 10D |
| | 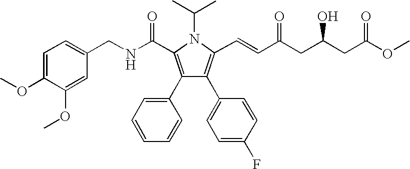 | 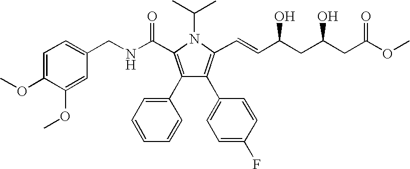 |
| | 7-[5-(3,4-Dimethoxy- | 7-[5-(3,4-Dimethoxy- |
| | benzylcarbamoyl)-3-(4- | benzylcarbamoyl)-3-(4- |
| | fluoro-phenyl)-1-isopropyl- | fluoro-phenyl)-1-isopropyl-4- |
| | 4-phenyl-1-pyrrol-2-yl]- | phenyl-1H-pyrrol-2-yl]-3,5- |
| | 3-hydroxy-5-oxo-hept-6- | dihydroxy-hept-6-enoic acid |
| | enoic acid methyl ester | methyl ester |
| MS | m/z 643 | m/z 645 |
| | (M + 1) | (M + 1) |
| Ex. | 10E | 11A |
| | 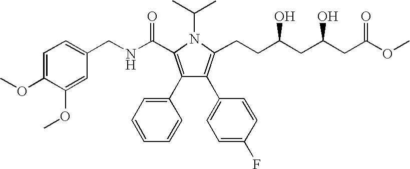 | 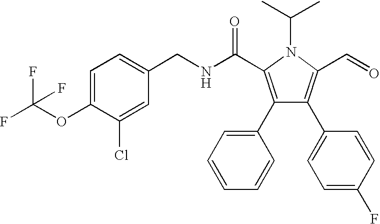 |
| | 7-[5-(3,4-Dimethoxy- | 4-(4-Fluoro- |
| | benzylcarbamoyl)-3-(4-fluoro- | phenyl)-5-formyl- |
| | phenyl)-1-isopropyl-4-phenyl- | 1-isopropyl-3- |
| | 1H-pyrrol-2-yl]-3,5- | phenyl-1- |
| | dihydroxy-heptanoic acid | pyrrole-2- |
| | methyl ester | carboxylic acid 3- |
| | | chloro-4- |
| | | trifluoromethoxy- |
| | | benzylamide |
| MS | m/z 647 | m/z 559 |
| | (M + 1) | (M + 1) |
| Ex. | | 11C |
| | 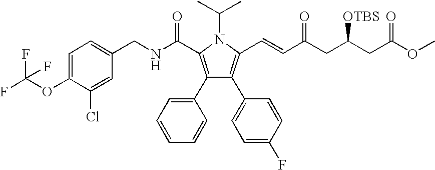 | 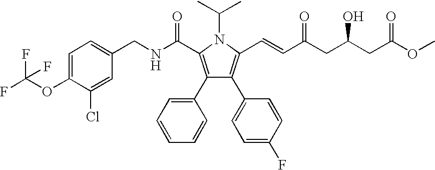 |
| | 3-(tert-Butyl-dimethyl- | 7-[5-(3-Chloro-4- |
| | silanyloxy)-7-[5-(3-chloro- | trifluoromethoxy- |
| | 4-trifluoromethoxy- | benzylcarbamoyl)-3-(4- |
| | benzylcarbamoyl)-3-(4- | fluoro-phenyl)-1-isopropyl- |
| | fluoro-phenyl)-1-isopropyl- | 4-phenyl-1-pyrrol-2-yl]- |
| | 4-phenyl-1-pyrrol-2-yl]-5- | 3-hydroxy-5-oxo-hept-6- |
| | oxo-hept-6-enoic acid | enoic acid methyl ester |
| | methyl ester | |
| MS | m/z 815 | m/z 701 |
| | (M + 1) | (M + 1) |
| Ex. | 11D | 11E |
| | 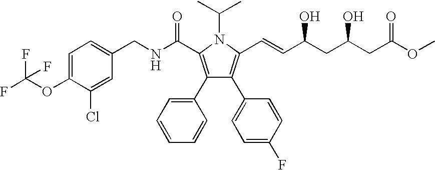 | 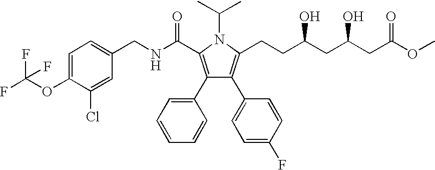 |
| | 7-[5-(3-Chloro-4- | 7-[5-(3-Chloro-4- |
| | trifluoromethoxy- | trifluoromethoxy- |
| | benzylcarbamoyl)-3-(4- | benzylcarbamoyl)-3-(4-fluoro- |
| | fluoro-phenyl)-1-isopropyl-4- | phenyl)-1-isopropyl-4-phenyl- |
| | phenyl-1-pyrrol-2-yl]-3,5- | 1H-pyrrol-2-yl]-3,5- |
| | dihydroxy-hept-6-enoic acid | dihydroxy-heptanoic acid |
| | methyl ester | methyl ester |
| MS | m/z 703 | m/z 705 |
| | (M + 1) | (M + 1) |
| Ex. | 12A | 12B |
| | 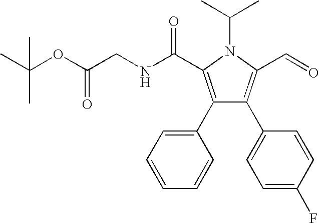 | 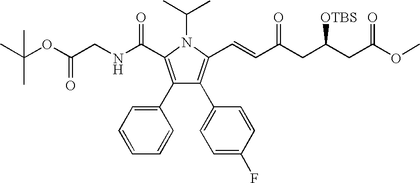 |
| | [4-(4-Fluoro- | 7-[5-(tert- |
| | phenyl)-5-formyl- | Butoxycarbonyl methyl- |
| | 1-isopropyl-3- | carbamoyl)-3-(4-fluoro- |
| | phenyl-1H- | phenyl)-1-isopropyl-4- |
| | pyrrole-2- | phenyl-1-pyrrol-2-yl]-3- |
| | carbonyl]-amino)- | (tert-butyl-dimethyl- |
| | acetic acid tert- | silanyloxy)-5-oxo-hept-6- |
| | butyl ester | enoic acid methyl ester |
| MS | m/z 465 | m/z 721 |
| | (M + 1) | (M + 1) |
| Ex. | 12C | 12D |
| | 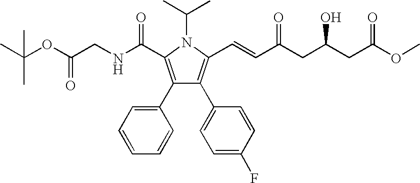 | 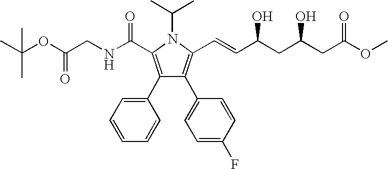 |
| | 7-[5-(tert- | 7-[5-(tert- |
| | Butoxycarbonylmethyl- | Butoxycarbonyl methyl- |
| | carbamoyl)-3-(4-fluoro- | carbamoyl)-3-(4-fluoro- |
| | phenyl)-1-isopropyl-4- | phenyl)-1-isopropyl-4- |
| | phenyl-1-pyrrol-2-yl]-3- | phenyl-1H-pyrrol-2-yl]-3,5- |
| | hydroxy-5-oxo-hept-6- | dihydroxy-hept-6-enoic acid |
| | enoic acid methyl ester | methyl ester |
| MS | m/z 607 | m/z 609 |
| | (M + 1) | (M + 1) |
| Ex. | 12E | 13A |
| | 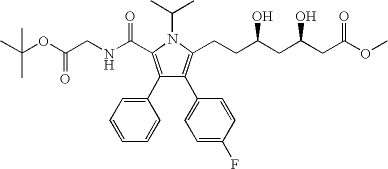 | 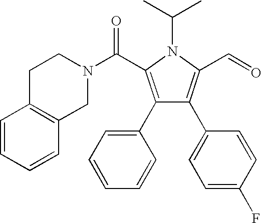 |
| | 7-[5-(tert- | 5-(3,4-Dihydro- |
| | Butoxycarbonylmethyl- | 1H-isoquinoline- |
| | carbamoyl)-3-(4-fluoro- | 2-carbonyl)-3-(4- |
| | phenyl)-1-isopropyl-4-phenyl- | fluoro-phenyl)-1- |
| | 1H-pyrrol-2-yl]-3,5- | isopropyl-4- |
| | dihydroxy-heptanoic acid | phenyl-1H- |
| | methyl ester | pyrrole-2- |
| | | carbaldehyde |
| MS | m/z 611 | 467 |
| | (M + 1) | |
| Ex. | 13B | 13C |
| | 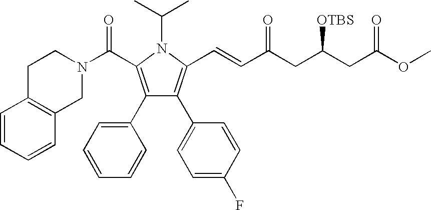 | 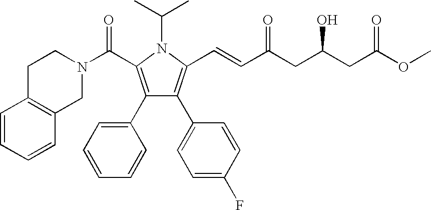 |
| | 3-(tert-Butyl-dimethyl- | 7-[5-(3,4-Dihydro-1H- |
| | silanyloxy)-7-[5-(3,4- | isoquinoline-2-carbonyl)-3- |
| | dihydro-1-isoquinoline-2- | (4-fluoro-phenyl)-1- |
| | carbonyl)-3-(4-fluoro- | isopropyl-4-phenyl-1H- |
| | phenyl)-1-isopropyl-4- | pyrrol-2-yl]-3-hydroxy-5- |
| | phenyl-1-pyrrol-2-yl]-5- | oxo-hept-6-enoic acid |
| | oxo-hept-6-enoic acid | methyl ester |
| | methyl ester | |
| MS = | 723 | 609 |
| Ex. | 13D | 13E |
| | 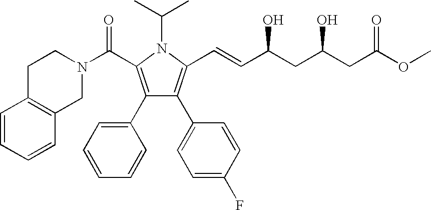 | 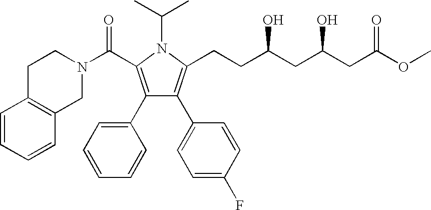 |
| | 7-[5-(3,4-Dihydro-1H- | 7-[5-(3,4-Dihydro-1H- |
| | isoquinoline-2-carbonyl)-3- | isoquinoline-2-carbonyl)-3-(4- |
| | (4-fluoro-phenyl)-1- | fluoro-phenyl)-1-isopropyl-4- |
| | isopropyl-4-phenyl-1- | phenyl-1-pyrrol-2-yl]-3,5- |
| | pyrrol-2-yl]-3,5-dihydroxy- | dihydroxy-heptanoic acid |
| | hept-6-enoic acid methyl | methy1 ester |
| | ester | |
| MS = | 611 | 613 |
| Ex. | 14A | 14B |
| | 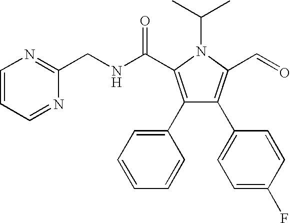 | 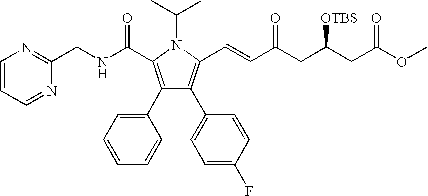 |
| | 4-(4-Fluoro- | 3-(tert-Butyl-dimethyl- |
| | phenyl)-5-formyl- | silanyloxy)-7-(3-(4-fluoro- |
| | 1-isopropyl-3- | phenyl)-1-isopropyl-4- |
| | phenyl-1H- | phenyl-5-[(pyrimidin-2- |
| | pyrrole-2- | ylmethyl)-carbamoyl]-1H- |
| | carboxylic acid | pyrrol-2-yl}-5-oxo-hept-6- |
| | (pyrimidin-2- | enoic acid methyl ester |
| | ylmethyl)-amide | |
| MS = | 443 | 698 |
| Ex. | 14C | 14D |
| | 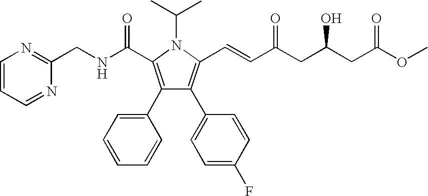 | 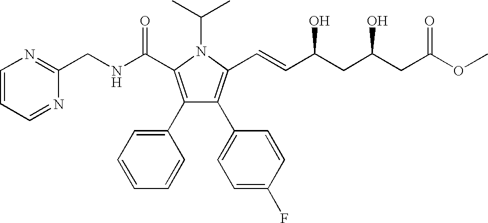 |
| | 7-{3-(4-Fluoro-phenyl)-1- | 7-{3-(4-Fluoro-phenyl)-1- |
| | isopropyl-4-phenyl-5- | isopropyl-4-phenyl-5- |
| | [(pyrimidin-2-ylmethyl)- | [(pyrimidin-2-ylmethyl)- |
| | carbamoyl]-1H-pyrrol-2- | carbamoyl]-1H-pyrrol-2-yl}- |
| | yl}-3-hydroxy-5-oxo-hept- | 3,5-dihydroxy-hept-6-enoic |
| | 6-enoic acid methyl ester | acid methyl ester |
| MS = | 585 | 587 |
| Ex. | 14E | 15A |
| | 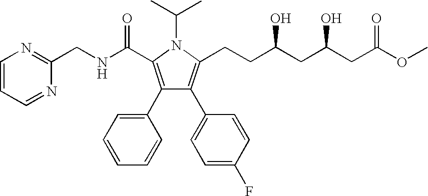 | 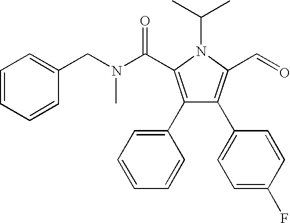 |
| | 7-{3-(4-Fluoro-phenyl)-1- | 4-(4-Fluoro- |
| | isopropyl-4-phenyl-5- | phenyl)-5-formyl- |
| | [(pyrimidin-2-ylmethyl)- | 1-isopropyl-3- |
| | carbamoyl]-1H-pyrrol-2-yl}- | phenyl-1H- |
| | 3,5-dihydroxy-heptanoic acid | pyrrole-2- |
| | methyl ester | carboxylic acid |
| | | benzyl-methyl- |
| | | amide |
| MS = | 589 | 455 |
| Ex. | 15B | 15C |
| | 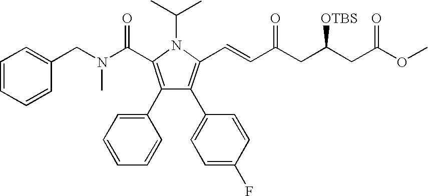 | 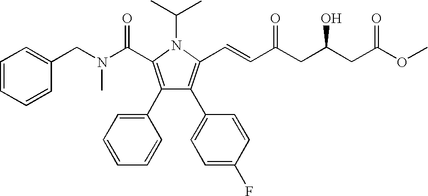 |
| | 7-[5-(Benzyl-methyl- | 7-[5-(Benzyl-methyl- |
| | carbamoyl)-3-(4-fluoro- | carbamoyl)-3-(4-fluoro- |
| | phenyl)-1-isopropyl-4- | phenyl)-1-isopropyl-4- |
| | phenyl-1-pyrrol-2-yl]-3- | phenyl-1-pyrrol-2-yl]-3- |
| | (tert-butyl-dimethyl- | hydroxy-5-oxo-hept-6- |
| | silanyloxy)-5-oxo-hept-6- | enoic acid methyl ester |
| | enoic acid methyl ester | |
| MS = | 711 | 597 |
| Ex. | 15D | 15E |
| | 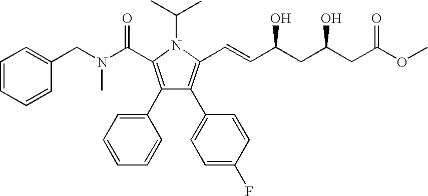 | 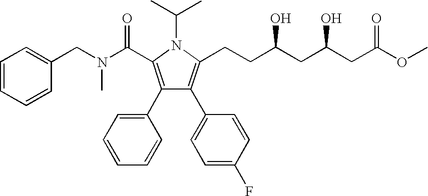 |
| | 7-[5-(Benzyl-methyl- | 7-[5-(Benzyl-methyl- |
| | carbamoyl)-3-(4-fluoro- | carbamoyl)-3-(4-fluoro- |
| | phenyl)-1-isopropyl-4- | phenyl)-1-isopropyl-4-phenyl- |
| | phenyl-1-pyrrol-2-yl]-3,5- | 1H-pyrrol-2-yl]-3,5- |
| | dihydroxy-hept-6-enoic acid | dihydroxy-heptanoic acid |
| | methyl ester | methyl ester |
| MS = | 599 | 601 |
| Ex. | 16A | 16B |
| | 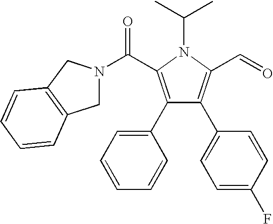 | 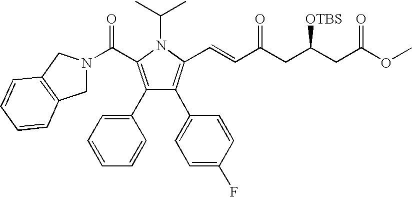 |
| | 5-(1,3-Dihydro- | 3-(tert-Butyl-dimethyl- |
| | isoindole-2- | silanyloxy)-7-[5-(1,3- |
| | carbonyl)-3-(4- | dihydro-isoindole-2- |
| | fluoro-phenyl)-1- | carbonyl)-3-(4-fluoro- |
| | isopropyl-4- | phenyl)-1-isopropyl-4- |
| | phenyl-1H- | phenyl-1H-pyrrol-2-yl]-5- |
| | pyrrole-2- | oxo-hept-6-enoic acid |
| | carbaldehyde | methyl ester |
| MS = | 453 | 709 |
| Ex. | 16C | 16D |
| | 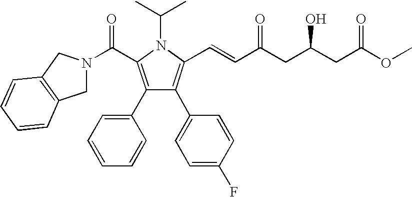 | 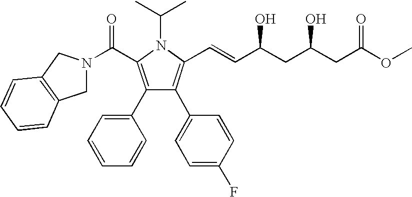 |
| | 7-[5-(1,3-Dihydro- | 7-[5-(1,3-Dihydro-isoindole- |
| | isoindole-2-carbonyl)-3-(4- | 2-carbonyl)-3-(4-fluoro- |
| | fluoro-phenyl)-1-isopropyl- | phenyl)-1-isopropyl-4- |
| | 4-phenyl-1H-pyrrol-2-yl]- | phenyl-1H-pyrrol-2-yl]-3,5- |
| | 3-hydroxy-5-oxo-hept-6- | dihydroxy-hept-6-enoic acid |
| | enoic acid methyl ester | methyl ester |
| MS = | 595 | 597 |
| Ex. | 16E | 17A |
| | 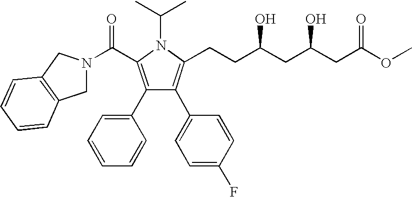 | 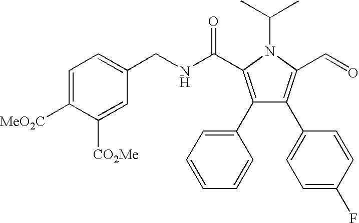 |
| | 7-[5-(1,3-Dihydro-isoindole-2- | [4-(4-Fluoro- |
| | carbonyl)-3-(4-fluoro-phenyl)- | phenyl)-5-formyl- |
| | 1-isopropyl-4-phenyl-1H- | 1-isopropyl-3- |
| | pyrrol-2-yl]-3,5-dihydroxy- | phenyl-1H- |
| | heptanoic acid methyl ester | pyrrole-2- |
| | | carbonyl]-amino}- |
| | | methyl)-2-methyl- |
| | | benzoic acid |
| | | methyl ester; |
| | | compound with |
| | | methanedione |
| MS = | 599 | 557 |
| Ex. | 17B | 17C |
| | 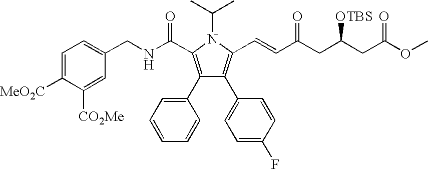 | 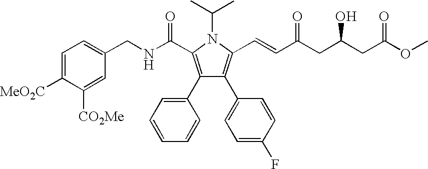 |
| | 5-({[5-[5-(tert-Butyl- | 5-({[4-(4-Fluoro-phenyl)-5- |
| | dimethyl-silanyloxy)-6- | (5-hydroxy-6- |
| | methoxycarbonyl-3-oxo- | methoxycarbonyl-3-oxo- |
| | hex-1-enyl]-4-(4-fluoro- | hex-1-enyl)-1-isopropyl-3- |
| | phenyl)-1-isopropyl-3- | phenyl-1-pyrrole-2- |
| | phenyl-1H-pyrrole-2- | carbonyl]-amino}-methyl)- |
| | carbonyl]-amino}-methyl)- | 2-methyl-benzoic acid |
| | 2-methyl-benzoic acid | methyl ester; compound |
| | methyl ester; compound with | with methanedione |
| | methanedione | |
| MS = | 813 | 699 |
| Ex. | 17D | 17E |
| | 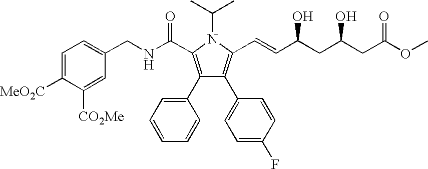 | 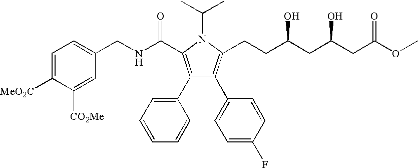 |
| | 5-({[5-(3,5-Dihydroxy-6- | 5-({[5-(3,5-Dihydroxy-6- |
| | methoxycarbonyl-hex-1- | methoxycarbonyl-hexyl)-4-(4- |
| | enyl)-4-(4-fluoro-phenyl)-1- | fluoro-phenyl)-1-isopropyl-3- |
| | isopropyl-3-phenyl-1H- | phenyl-1H-pyrrole-2- |
| | pyrrole-2-carbonyl]-amino}- | carbonyl]-amino}-methyl)-2- |
| | methyl)-2-methyl-benzoic | methyl-benzoic acid methyl |
| | acid methyl ester; compound | ester; compound with |
| | with methanedione | methanedione |
| MS = | 701 | 703 |
| Ex. | 18A | 18B |
| | 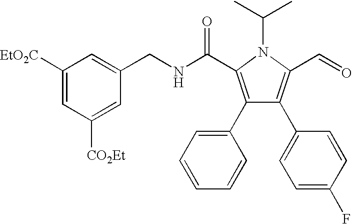 | 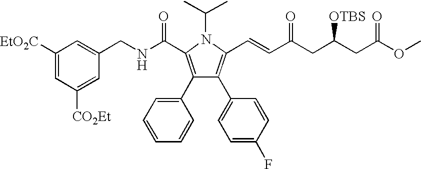 |
| | 5-({[4-(4-Fluoro- | 5-({[5-[5-(tert-Butyl- |
| | phenyl)-5-formyl- | dimethyl-silanyloxy)-6- |
| | 1-isopropyl-3- | methoxycarbonyl-3-oxo- |
| | phenyl-1H- | hex-1-enyl]-4-(4-fluoro- |
| | pyrrole-2- | phenyl)-1-isopropyl-3- |
| | carbonyl]-amino}- | phenyl-1H-pyrrole-2- |
| | methyl)- | carbonyl]-amino}-methyl)- |
| | isophthalic acid | isophthalic acid diethyl ester |
| | diethyl ester | |
| MS = | 585 | 841 |
| Ex. | 18C | 18D |
| | 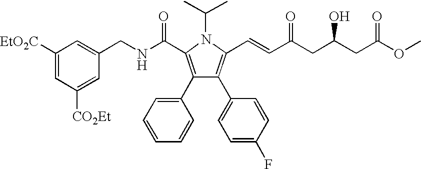 | 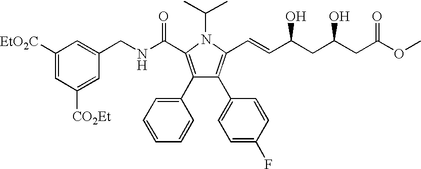 |
| | 5-({[4-(4-Fluoro-phenyl)-5- | 5-({[5-(3,5-Dihydroxy-6- |
| | (5-hydroxy-6- | methoxycarbonyl-hex-1- |
| | methoxycarbonyl-3-oxo- | enyl)-4-(4-fluoro-phenyl)-1- |
| | hex-1-enyl)-1-isopropyl-3- | isopropyl-3-phenyl-1H- |
| | phenyl-1H-pyrrole-2- | pyrrole-2-carbonyl]-amino}- |
| | carbonyl]-amino}-methyl)- | methyl)-isophthalic acid |
| | isophthalic acid diethyl | diethyl ester |
| | ester | |
| MS = | 727 | 729 |
| Ex. | 18E | 19A |
| | 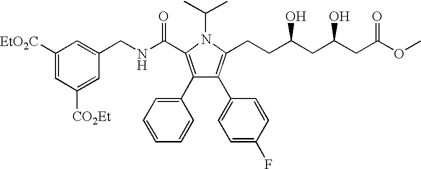 | 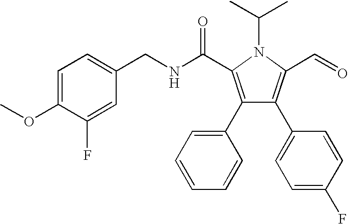 |
| | 5-({[5-(3,5-Dihydroxy-6- | 4-(4-Fluoro- |
| | methoxycarbonyl-hexyl)-4-(4- | phenyl)-5-formyl- |
| | fluoro-phenyl)-1-isopropyl-3- | 1-isopropyl-3- |
| | phenyl-1H-pyrrole-2- | phenyl-1H- |
| | carbonyl]-amino}-methyl)- | pyrrole-2- |
| | isophthalic acid diethyl ester | carboxylic acid 3- |
| | | fluoro-4-methoxy- |
| | | benzylamide |
| MS = | 731 | 489 |
| Ex. | 19B | 19C |
| | 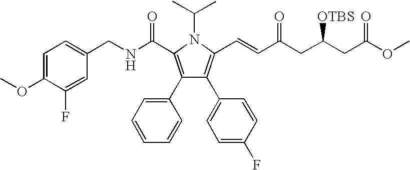 | 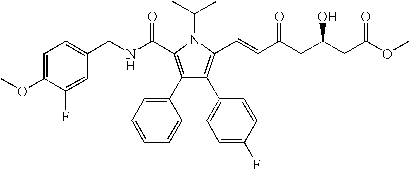 |
| | 3-(tert-Butyl-dimethyl- | 7-[5-(3-Fluoro-4-methoxy- |
| | silanyloxy)-7-[5-(3-fluoro-4- | benzylcarbamoyl)-3-(4- |
| | methoxy-benzylcarbamoyl)- | fluoro-phenyl)-1-isopropyl- |
| | 3-(4-fluoro-phenyl)-1- | 4-phenyl-1-pyrrol-2-yl]- |
| | isopropyl-4-phenyl-1- | 3-hydroxy-5-oxo-hept-6- |
| | pyrrol-2-yl]-5-oxo-hept-6- | enoic acid methyl ester |
| | enoic acid methyl ester | |
| MS = | 745 | 631 |
| Ex. | 19D | 19E |
| | 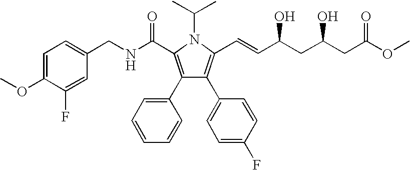 | 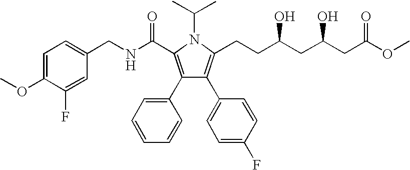 |
| | 7-[5-(3-Fluoro-4-methoxy- | 7-[-(3-Fluoro-4-methoxy- |
| | benzylcarbamoyl)-3-(4- | benzylcarbamoyl)-3-(4-fluoro- |
| | fluoro-phenyl)-1-isopropyl-4- | phenyl)-1-isopropyl-4-phenyl- |
| | phenyl-1-pyrrol-2-yl]-3,5- | 1H-pyrrol-2-yl]-3,5- |
| | dihydroxy-hept-6-enoic acid | dihydroxy-heptanoic acid |
| | methyl ester | methyl ester |
| MS = | 633 | 635 |
| Ex. | 20A | 20B |
| | 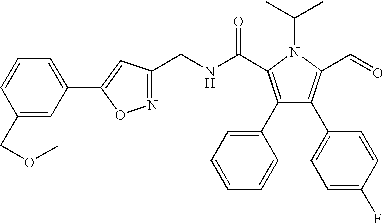 | 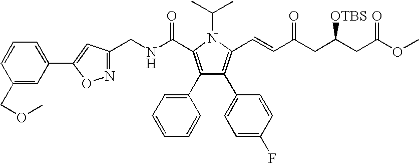 |
| | 4-(4-Fluoro- | 3-(tert-Butyl-dimethyl- |
| | phenyl)-5-formyl- | silanyloxy)-7-(3-(4-fluoro- |
| | 1-isopropyl-3- | phenyl)-1-isopropyl-5-{[5- |
| | pyrrole-2- | isoxazol-3-ylmethyl]- |
| | carboxylic acid [5- | carbamoyl}-4-phenyl-1H- |
| | (3- | pyrrol-2-yl)-5-oxo-hept-6- |
| | methoxymethyl- | enoic acid methyl ester |
| | phenyl)-isoxazol | |
| | 3-ylmethyl]-amide | |
| MS = | 552 | 809 |
| Ex. | 20C | 20D |
| | 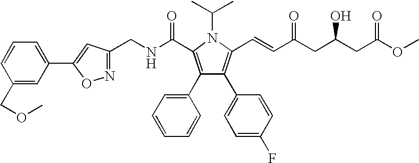 | 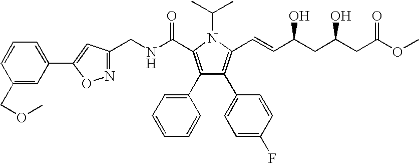 |
| | 7-(3-(4-Fluoro-phenyl)-1- | 7-(3-(4-Fluoro-phenyl)-1- |
| | isopropyl-5-{[5-(3- | isopropyl-5-{[5-(3- |
| | methoxymethyl-phenyl)- | methoxymethyl-phenyl)- |
| | isoxazol-3-ylmethyl]- | isoxazol-3-ylmethyl]- |
| | carbamoyl}-4-phenyl-1H- | carbamoyl}-4-phenyl-1H- |
| | pyrrol-2-yl)-3-hydroxy-5- | pyrrol-2-yl)-3,5-dihydroxy- |
| | oxo-hept-6-enoic acid | hept-6-enoic acid methyl |
| | methyl ester | ester |
| MS = | 694 | 696 |
| Ex. | 20E | 21A |
| | 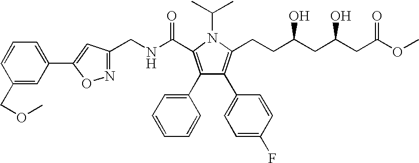 | 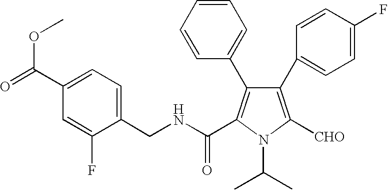 |
| | 7-(3-(4-Fluoro-phenyl)-1- | 3-Fluoro-4-(([4- |
| | isopropyl-5-{[5-(3- | (4-fluoro-phenyl)- |
| | methoxymethyl-phenyl)- | 5-formyl-1- |
| | isoxazol-3-ylmethyl]- | isopropyl-3- |
| | carbamoyl}-4-phenyl-1- | phenyl-1H- |
| | pyrrol-2-yl)-3,5-dihydroxy- | pyrrole-2- |
| | heptanoic acid methyl ester | carbonyl]-amino}- |
| | | methyl)-benzoic |
| | | acid methyl ester |
| MS = | 698 | 517 |
| Ex. | 21B | 21C |
| | 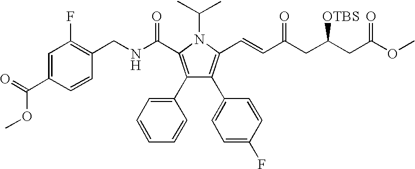 | 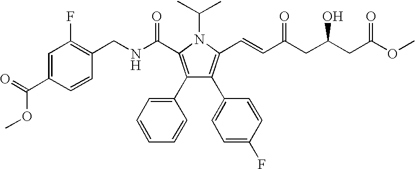 |
| | 4-({[5-[5-(tert-Butyl- | 3-Fluoro-4-({[4-(4-fluoro- |
| | dimethyl-silanyloxy)-6- | phenyl)-5-(5-hydroxy-6- |
| | methoxy carbonyl-3-oxo- | methoxycarbonyl-3-oxo- |
| | hex-1-enyl]-4-(4-fluoro- | hex-1-enyl)-1-isopropyl-3- |
| | phenyl)-1-isopropyl-3- | phenyl-1H-pyrrole-2- |
| | phenyl-1H-pyrrole-2- | carbonyl]-amino}-methyl)- |
| | carbonyl]-amino}-methyl)- | benzoic acid methyl ester |
| | 3-fluoro-benzoic acid methyl | |
| | ester | |
| MS = | 773 | 659 |
| Ex. | 21D | 21E |
| | 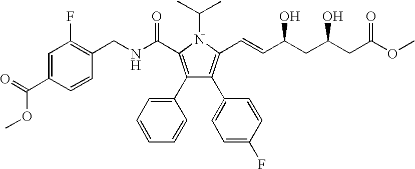 | 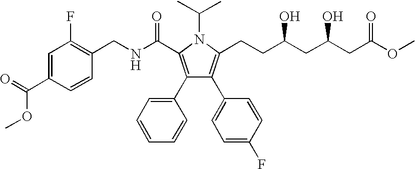 |
| | 4-({[5-(3,5-Dihydroxy-6- | 4-({[5-(3,5-Dihydroxy-6- |
| | methoxycarbonyl-hex-1- | methoxycarbonyl-hexyl)-4-(4- |
| | enyl)-4-(4-fluoro-phenyl)-1- | fluoro-phenyl)-1-isopropyl-3- |
| | isopropyl-3-phenyl-1H- | phenyl-1H-pyrrole-2- |
| | pyrrole-2-carbonyl]-amino}- | carbonyl]-amino}-methyl)-3- |
| | methyl)-3-fluoro-benzoic acid | fluoro-benzoic acid methyl |
| | methyl ester ester | |
| MS = | 661 | 663 |
| Ex. | 22A | 22B |
| | 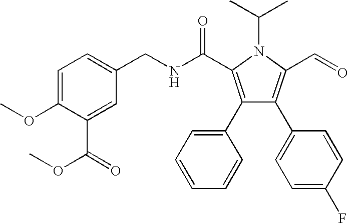 | 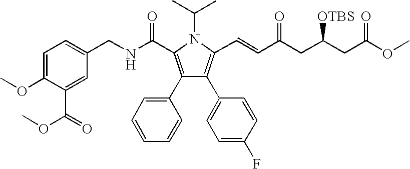 |
| | 5-({[4-(4-Fluoro- | 5-({[5-[5-(tert-Butyl- |
| | phenyl)-5-formyl- | dimethyl-silanyloxy)-6- |
| | 1-isopropyl-3- | methoxycarbonyl-3-oxo- |
| | phenyl-1H- | hex-1-enyl]-4-(4-fluoro- |
| | pyrrole-2- | phenyl)-1-isopropyl-3- |
| | carbonyl]-amino}- | phenyl-1H-pyrrole-2- |
| | methyl)-2- | carbonyl]-amino}-methyl)- |
| | methoxy-benzoic | 2-methoxy-benzoic acid |
| | acid methyl ester | methyl ester |
| MS | m/z 529 | m/z 785 |
| | (M + 1) | (M + 1) |
| Ex. | 22C | 22D |
| | 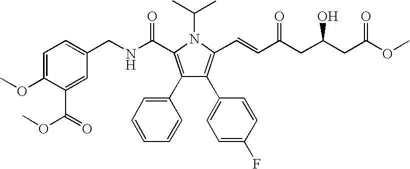 | 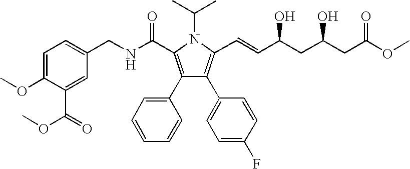 |
| | 5-({[4-(4-Fluoro-phenyl)-5- | 5-(}[5-(3,5-Dihydroxy-6- |
| | (5-hydroxy-6- | methoxycarbonyl-hex-1- |
| | methoxycarbonyl-3-oxo- | enyl)-4-(4-fluoro-phenyl)-1- |
| | hex-1-enyl)-1-isopropyl-3- | isopropyl-3-phenyl-1H- |
| | phenyl-1H-pyrrole-2- | pyrrole-2-carbonyl]-amino}- |
| | carbonyl]-amino}-methyl)- | methyl)-2-methoxy-benzoic |
| | 2-methoxy-benzoic acid | acid methyl ester |
| | methyl ester | |
| MS | m/z 671 | m/z 673 |
| | (M + 1) | (M + 1) |
| Ex. | 22E | 23A |
| | 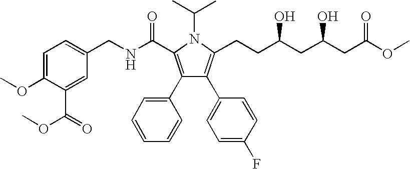 | 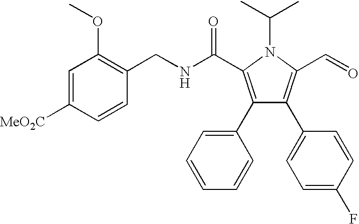 |
| | 5-({[5-(3,5-Dihydroxy-6- | 4-({[4-(4-Fluoro- |
| | methoxycarbonyl-hexyl)-4-(4- | phenyl)-5-formyl- |
| | fluoro-phenyl)-1-isopropyl-3- | 1-isopropyl-3- |
| | phenyl-1H-pyrrole-2- | phenyl-1H- |
| | carbonyl]-amino}-methyl)-2- | pyrrole-2- |
| | methoxy-benzoic acid methyl | carbonyl]-amino}- |
| | ester | methyl)-3- |
| | | methoxy-benzoic |
| | | acid methyl ester |
| MS | m/z 675 | MS = 529 |
| | (M + 1) | |
| Ex. | 23B | 23C |
| | 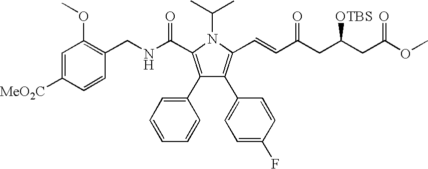 | 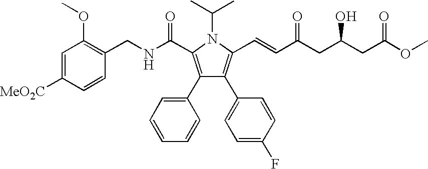 |
| | 4-({[5-[5-(tert-Butyl- | 4-({[4-(4-Fluoro-phenyl)-5- |
| | dimethyl-silanyloxy)-6- | (5-hydroxy-6- |
| | methoxycarbonyl-3-oxo- | methoxycarbony[3-oxo- |
| | hex-1-enyl]-4-(4-fluoro- | hex-1-enyl)-1-isopropyl-3- |
| | phenyl)-1-isopropyl-3- | phenyl-1H-pyrrole-2- |
| | phenyl-1H-pyrrole-2- | carbonyl]-amino}-methyl)- |
| | carbonyl]-amino}-methyl)- | 3-methoxy-benzoic acid |
| | 3-methoxy-benzoic acid | methyl ester |
| | methyl ester | |
| MS = | 785 | 671 |
| Ex. | 23D | 23E |
| | 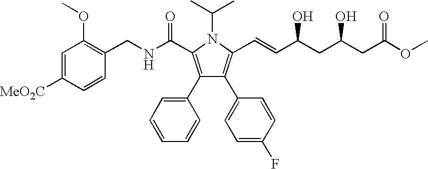 | 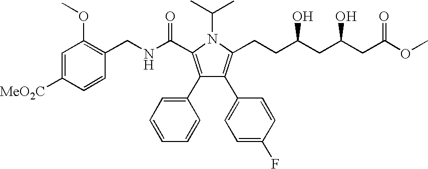 |
| | 4-(}[5-(3,5-Dihydroxy-6- | 4-({[5-(3,5-Dihydroxy-6- |
| | methoxycarbonyl-hex-1- | methoxycarbonyl-hexyl)-4-(4- |
| | enyl)-4-(4-fluoro-phenyl)-1- | fluoro-phenyl)-1-isopropyl-3- |
| | isopropyl-3-phenyl-1H- | phenyl-1H-pyrrole-2- |
| | pyrrole-2-carbonyl]-amino}- | carbonyl]-amino}-methyl)-3- |
| | methyl)-3-methoxy-benzoic | methoxy-benzoic acid methyl |
| | acid methyl ester | ester |
| MS = | 673 | 675 |
| Ex. | 24A | 24B |
| | 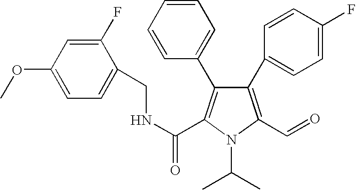 | 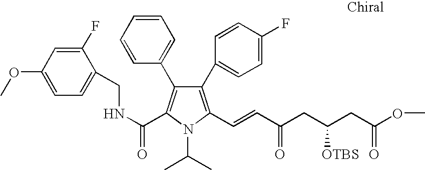 |
| | 4-(4-Fluoro- | 3-(tert-Butyl-dimethyl- |
| | phenyl)-5-formyl- | silanyloxy)-7-[5-(2-fluoro-4- |
| | 1-isopropyl-3- | methoxy-benzylcarbamoyl)- |
| | phenyl-1H- | 3-(4-fluoro-phenyl)-1- |
| | pyrrole-2- | isopropyl-4-phenyl-1H- |
| | carboxylic acid 2- | pyrrol-2-yl]-5-oxo-hept-6- |
| | fluoro-4-methoxy- | enoic acid methyl ester |
| | benzylamide | |
| MS = | 489 | 745 |
| Ex. | 24C | 24D |
| | 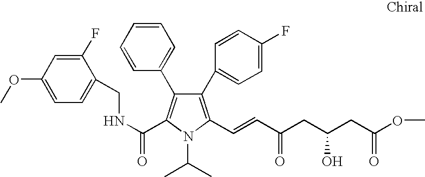 | 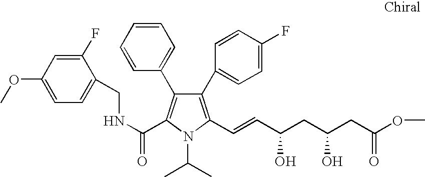 |
| | 7-[5-(2-Fluoro-4-methoxy- | 7-[5-(2-Fluoro-4-methoxy- |
| | benzylcarbamoyl)-3-(4- | benzylcarbamoyl)-3-(4- |
| | fluoro-phenyl)-1-isopropyl- | fluoro-phenyl)-1-isopropyl-4- |
| | 4-phenyl-1H-pyrrol-2-yl]- | phenyl-1-pyrrol-2-yl]-3,5- |
| | 3-hydroxy-5-oxo-hept-6- | dihydroxy-hept-6-enoic acid |
| | enoic acid methyl ester | methyl ester |
| MS = | 631 | 633 |
| Ex. | 24E | 25A |
| | 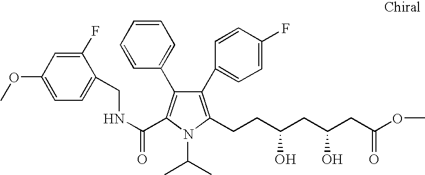 | 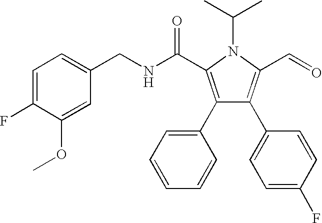 |
| | 7-115-(2-Fluoro-4-methoxy- | 4-(4-Fluoro- |
| | benzylcarbamoyl)-3-(4-fluoro- | phenyl)-5-formyl- |
| | phenyl)-1-isopropyl-4-phenyl- | 1-isopropyl-3- |
| | 1H-pyrrol-2-yl]-3,5- | phenyl-1H- |
| | dihydroxy-heptanoic acid | pyrrole-2- |
| | methyl ester | carboxylic acid 4- |
| | | fluoro-3-methoxy- |
| | | benzylamide |
| MS = | 635 | 489 |
| Ex. | 25B | 25C |
| | 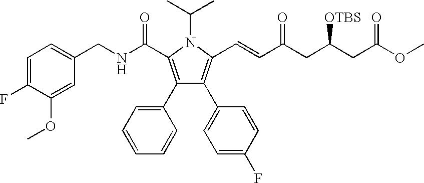 | 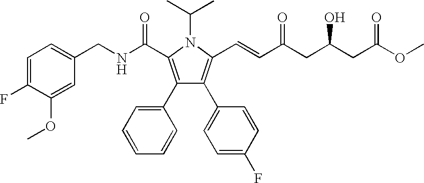 |
| | 3-(tert-Butyl-dimethyl- | 7-[5-(4-Fluoro-3-methoxy- |
| | silanyloxy)-7-[5-(4-fluoro-3- | benzylcarbamoyl)-3-(4- |
| | methoxy-benzylcarbamoyl)- | fluoro-phenyl)-1-isopropyl- |
| | 3-(4-fluoro-phenyl)-1- | 4-phenyl-1-pyrrol-2-yl]- |
| | isopropyl-4-phenyl-1- | 3-hydroxy-5-oxo-hept-6- |
| | pyrrol-2-yl]-5-oxo-hept-6- | enoic acid methyl ester |
| | enoic acid methyl ester | |
| MS = | 745 | 631 |
| Ex. | 25D | 25E |
| | 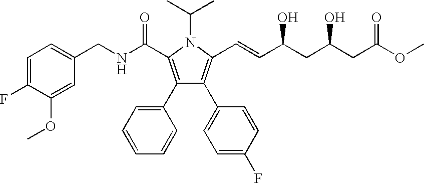 | 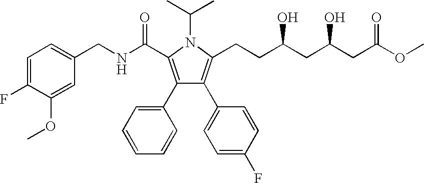 |
| | 7-[5-(4-Fluoro-3-methoxy- | 7-[5-(4-Fluoro-3-methoxy- |
| | benzylcarbamoyl)-3-(4- | benzylcarbamoyl)-3-(4-fluoro- |
| | fluoro-phenyl)-1-isopropyl-4- | phenyl)-1-isopropyl-4-phenyl- |
| | phenyl-1H-pyrrol-2-yl]-3,5- | 1H-pyrrol-2-yl]-3,5- |
| | dihydroxy-hept-6-enoic acid | dihydroxy-heptanoic acid |
| | methyl ester | methyl ester |
| MS = | 633 | 635 |
| Ex. | 26A | 26B |
| | 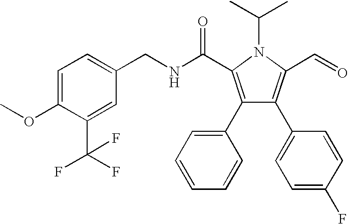 | 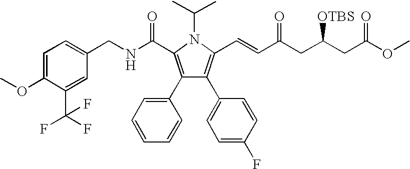 |
| | 4-(4-Fluoro- | 3-(tert-Butyl-dimethyl- |
| | phenyl)-5-formyl- | silanyloxy)-7-[3-(4-fluoro- |
| | 1-isopropyl-3- | phenyl)-1-isopropyl-5-(4- |
| | phenyl-1- | methoxy-3-trifluoromethyl- |
| | pyrrole-2- | benzylcarbamoyl)-4-phenyl- |
| | carboxylic acid 4- | 1H-pyrrol-2-yl]-5-oxo-hept- |
| | methoxy-3- | 6-enoic acid methyl ester |
| | trifluoromethyl- | |
| | benzylamide | |
| MS | m/z 539 | m/z 795 |
| | (M + 1) | (M + 1) |
| Ex. | 26C | 26D |
| | 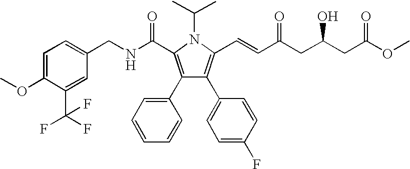 | 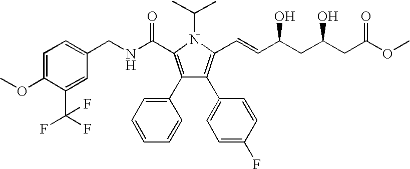 |
| | 7-[3-(4-Fluoro-phenyl)-1- | 7-[3-(4-Fluoro-phenyl)-1- |
| | isopropyl-5-(4-methoxy-3- | isopropyl-5-(4-methoxy-3- |
| | trifluoromethyl- | trifluoromethyl- |
| | benzylcarbamoyl)-4- | benzylcarbamoyl)-4-phenyl- |
| | phenyl-1-pyrrol-2-yl]-3- | 1H-pyrrol-2-yl]-3,5- |
| | hydroxy-5-oxo-hept-6- | dihydroxy-hept-6-enoic acid |
| | enoic acid methyl ester | methyl ester |
| MS | m/z 681 | m/z 683 |
| | (M + 1) | (M + 1) |
| Ex. | 26E | 27A |
| | 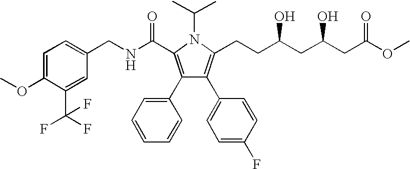 | 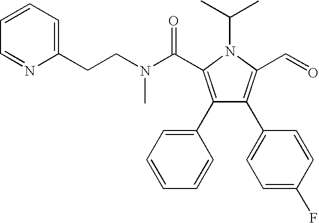 |
| | 7-[3-(4-Fluoro-phenyl)-1- | 4-(4-Fluoro- |
| | isopropyl-5-(4-methoxy-3- | phenyl)-5-formyl- |
| | trifluoromethyl- | 1-sopropyl-3- |
| | benzylcarbamoyl)-4-phenyl- | phenyl-1H- |
| | 1H-pyrrol-2-yl]-3,5- | pyrrole-2- |
| | dihydroxy-heptanoic acid | carboxylic acid |
| | methyl ester | methyl-(2-pyridin- |
| | | 2-yl-ethyl)-amide |
| MS | m/z 685 | MS = 470 |
| | (M + 1) | (M + 1) |
| Ex. | 27B | 27C |
| | 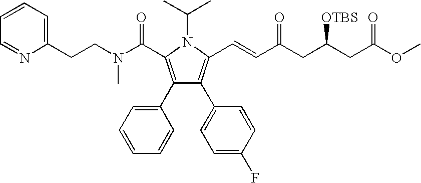 | 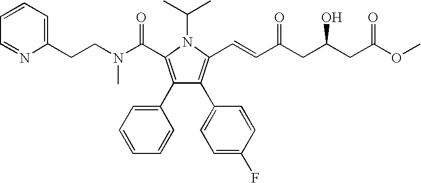 |
| | 3-(tert-Butyl-dimethyl- | 7-{3-(4-Fluoro-phenyl)-1- |
| | silanyloxy)-7-{3-(4-fluoro- | isopropyl-5-[methyl-(2- |
| | phenyl)-1-isopropyl-5- | pyridin-2-yl-ethyl)- |
| | [methyl-(2-pyridin-2-yl- | carbamoyl]-4-phenyl-1H- |
| | ethyl)-carbamoyl]-4-phenyl- | pyrrol-2-yl}-3-hydroxy-5- |
| | 1H-pyrrol-2-yl}-5-oxo-hept- | oxo-hept-6-enoic acid |
| | 6-enoic acid methyl ester | methyl ester |
| MS = | 726 | 612 |
| Ex. | 27D | 27E |
| | 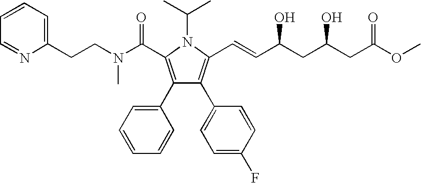 | NA |
| | 7-{3-(4-Fluoro-phenyl)-1- | |
| | isopropyl-5-[methyl-(2- | |
| | pyridin-2-yl-ethyl)- | |
| | carbamoyl]-4-phenyl-1- | |
| | pyrrol-2-yl}-3,5-dihydroxy- | |
| | hept-6-enoic acid methyl | |
| | ester | |
| MS = | 614 | |
| Ex. | 27A | 28B |
| | 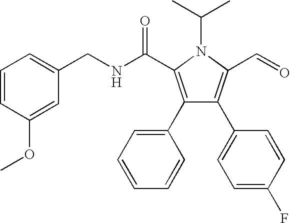 | 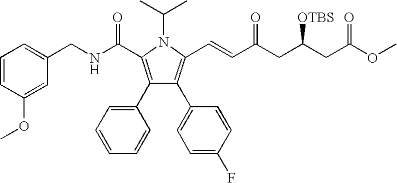 |
| | 4-(4-Fluoro- | 3-(tert-Butyl-dimethyl- |
| | phenyl)-5-formyl- | silanyloxy)-7-[3-(4-fluoro- |
| | 1-isopropyl-3- | phenyl)-1-isopropyl-5-(3- |
| | phenyl-1H- | methoxy-benzylcarbamoyl)- |
| | pyrrole-2- | 4-phenyl-1-pyrrol-2-yl]-5- |
| | carboxylic acid 3- | oxo-hept-6-enoic acid |
| | methoxy- | methyl ester |
| | benzylamide | |
| MS | m/z 471 | m/z 727 |
| | (M + 1) | (M + 1) |
| Ex. | 28C | 28D |
| | 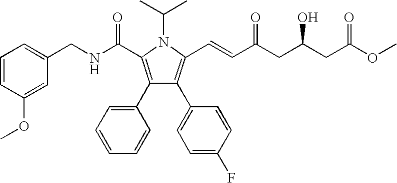 | 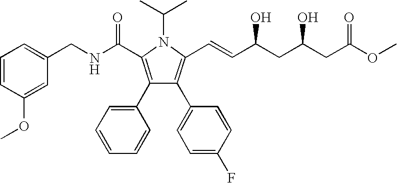 |
| | 7-[3-(4-Fluoro-phenyl)-1- | 7-113-(4-Fluoro-phenyl)-1- |
| | isopropyl-5-(3-methoxy- | isopropyl-5-(3-methoxy- |
| | benzylcarbamoyl)-4- | benzylcarbamoyl)-4-phenyl- |
| | phenyl-1-pyrrol-2-yl]-3- | 1H-pyrrol-2-yl]-3,5- |
| | hydroxy-5-oxo-hept-6- | dihydroxy-hept-6-enoic acid |
| | enoic acid methyl ester | methyl ester |
| MS | m/z 613 | m/z 615 |
| | (M + 1) | (M + 1) |

Note to Table III: MS-m/z (M +1)

### Example 11

### (3R,5R)-7-[3,4-Bis-(4-fluoro-phenyl)-1-isopropyl-5-(4-sulfamoyl-phenylcarbamoyl)-1H-pyrrol-2-yl]-3,5-dihydroxy-heptanoic Acid Sodium Salt

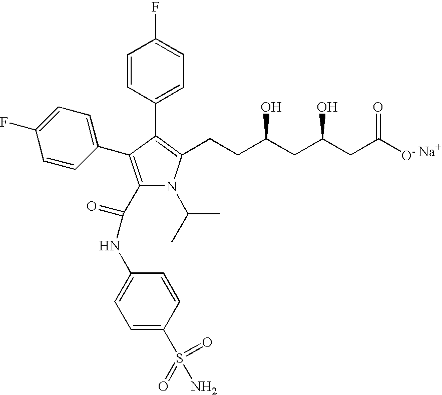 Step A

### 1-Fluoro-4-[1-nitro-2-(4-fluoro-phenyl)-vinyl]-benzene

To a solution of 1-fluoro-4-nitromethyl-benzene (7.92 g, 51.1 mmol) in acetic acid (13 mL) was added 4-fluorobenzylidene-butyl-amine (9.15 g, 51.16 mmol). The mixture was allowed to stir at room temperature overnight and the yellow crystalline solid was formed. The solid was filtered and washed with water twice and dried in vacuo to give 10.9 g (82%) of the desired product as a yellow solid: mp 107-109° C.; MS(APCI⁻): m/z 261.0 (M−H); Anal. Calcd for C₁₄H₉F₂N₁O₂: C, 64.37; H, 3.47; N, 5.36. Found: C, 64.20; H, 3.29; N, 5.39.

Step B

### 3,4-Bis-(4-fluoro-phenyl)-1H-pyrrole-2-carboxylic Acid Ethyl Ester

To a mixture of 1-fluoro-4-[1-nitro-2-(4-fluoro-phenyl)-vinyl]-benzene prepared from step A (5.4 g, 21 mmol) and ethyl isocyanoacetate (3.4 mL, 31 mmol) in THF (60 mL) was added DBU (4.6 mL, 31 mmol) slowly over 10 minutes under a nitrogen atmosphere. The resulting mixture was stirred at room temperature overnight and partitioned between ethyl acetate and water. The organic phase was separated and washed with water and brine, dried over Na₂SO₄ and filtered. The filtrate was concentrated in vacuo to give a residue, which was purified by chromatography (2%-12% ethyl acetate in hexanes) to give 2.6 g (38%) of the desired product as an off-white solid: mp 157-159° C.; MS(APCI⁻): m/z 326.1 (M−H); Anal. Calcd for C₁₉H₁₅F₂N₁O₂: C, 69.72; H, 4.62; N, 4.28. Found: C, 69.47; H, 4.25; N, 4.28.

Step C

### 3,4-Bis-(4-fluoro-phenyl)-1-isopropyl-1H-pyrrole-2-carboxylic Acid Ethyl Ester

To a mixture of pre-crashed potassium hydroxide (4.5 g, 81 mmol) in DMSO (34 mL) was added 3,4-bis-(4-fluoro-phenyl)-1H-pyrrole-2-carboxylic acid ethyl ester prepared from step B (5.3 g, 16 mmol). The mixture was stirred at room temperature under a nitrogen atmosphere for 45 min and then isopropyl iodide (4.86 mL, 48.6 mmol) was added dropwise. After addition was complete, the resulting mixture was stirred at room temperature for 45 min and partitioned between diethyl ether and water. The organic phase was separated and washed with water three times and brine, dried over Na₂SO₄ and filtered. The filtrate was concentrated in vacuo to give a residue, which was purified by chromatography (2%-10% ethyl acetate in hexanes) to give 3.72 g (62%) of the desired product as a white solid: mp 104-105° C.; MS(APCI⁺): m/z 370.1 (MH⁺); Anal. Calcd for C₂₂H₂₁F₂N₁O₂: C, 71.53; H, 5.73; N, 3.79. Found: C, 71.60; H, 5.87; N, 3.69.

Step D

### 3,4-Bis-(4-fluoro-phenyl)-5-formyl-1-isopropyl-1H-pyrrole-2-carboxylic Acid Ethyl Ester

To POCl₃ (1.22 mL, 13.1 mmol) was added anhydrous DMF (1.01 mL, 13.1 mmol) at −78° C. under a nitrogen atmosphere. After the mixture was stirred for 0.5 h, dichloroethane (6 mL) was added dropwise over 5 minutes followed by a solution of 3,4-bis-(4-fluoro-phenyl)-1-isopropyl-1H-pyrrole-2-carboxylic acid ethyl ester prepared from step C (3.72 g, 10.1 mmol) in dichloroethane (6 mL) dropwise over 10 minutes. At the end of the addition the cooling bath was removed and the reaction was heated at reflux for 1 h. The mixture was cooled, and then cooled in an ice bath. Saturated sodium acetate solution (5 mL) was added slowly, and the ice bath was removed. The solution was again brought to reflux for 1 h and then partitioned between ethyl acetate and water. The organic phase was separated and washed with water and brine, dried over Na₂SO₄ and filtered. The filtrate was concentrated in vacuo to give a residue, which was purified by chromatography (2%-10% ethyl acetate in hexanes) to give 1.8 g (45%) of the desired product as a white solid: mp 92-94° C.; MS(APCI⁻): m/z 397.1 (M−H); Anal. Calcd for C₃₃H₂₁F₂N₁O₃: C, 69.51; H, 5.33; N, 3.52. Found: C, 69.31; H, 5.07; N, 3.43.

Step E

### 3,4-Bis-(4-fluoro-phenyl)-5-formyl-1-isopropyl-1H-pyrrole-2-carboxylic Acid

To a solution of 3,4-bis-(4-fluoro-phenyl)-5-formyl-1-isopropyl-1H-pyrrole-2-carboxylic acid ethyl ester prepared from step D (1.8 g, 4.5 mmol) in methanol (25 mL) was added a solution of sodium hydroxide (0.73 g, 18.1 mmol) in water (3 mL). The mixture was stirred at 60° C. for 2 h. TLC showed that the reaction was complete. The mixture was cooled, and partitioned between ethyl acetate and 1N HCl solution. The organic phase was separated and washed with water and brine, dried over Na₂SO₄ and filtered. The filtrate was concentrated in vacuo to give 1.7 g (100%) of the desired product as a white solid: mp 228-230° C.; MS(APCI⁻): m/z 368.1 (M−H); Anal. Calcd for C₂₁H₁₇F₂N₁O₃: C, 68.29; H, 4.64; N, 3.79. Found: C, 67.91; H, 4.38; N, 3.67.

Step F

### 3,4-Bis-(4-fluoro-phenyl)-5-formyl-1-isopropyl-1H-pyrrole-2-carboxylic Acid (4-sulfamoyl-phenyl)-amide

A mixture of 3,4-bis-(4-fluoro-phenyl)-5-formyl-1-isopropyl-1H-pyrrole-2-carboxylic acid prepared from step E (0.8 g, 2.2 mmol) in thionyl chloride (5 mL) was heated at reflux for 1 h. The resulting mixture was concentrated in vacuo to give a residue, which was dried in vacuo for 1 h. The crude acid chloride was dissolved in THF (10 mL) under a nitrogen atmosphere. The mixture was cooled in an ice bath and 4-sulfamoyl-aniline (0.75 g, 4.33 mmol) was added followed by triethylamine (0.78 mL, 5.6 mmol). The mixture was stirred at room temperature overnight and partitioned between ethyl acetate and water. The organic phase was separated and washed with 1N HCl, NaHCO₃ and brine, dried over Na₂SO₄ and filtered. The filtrate was concentrated in vacuo to give a residue, which was purified by chromatography (5%-40% ethyl acetate in hexanes) to give 0.65 g (57%) of the desired product as a light yellow solid: mp 194-195° C.; MS(APCI⁻): m/z 522.2 (M−H); Anal. Calcd for C₂₇H₂₃F₂N₃O₄S₁.0.3EtOAc: C, 61.27; H, 4.60; N, 7.50. Found: C, 61.58; H. 4.65; N, 7.64.

Step G

### (3R)-7-[3,4-Bis-(4-fluoro-phenyl)-1-isopropyl-5-(4-sulfamoyl-phenylcarbamoyl)-1H-pyrrol-2-yl]-3-(tert-butyl-dimethyl-silanyloxy)-5-oxo-hept-6-enoic Acid Methyl Ester

To a mixture of 3,4-bis-(4-fluoro-phenyl)-5-formyl-1-isopropyl-1H-pyrrole-2-carboxylic acid (4-sulfamoyl-phenyl)-amide prepared from step F (0.53 g, 1.01 mmol) in toluene (20 mL) at room temperature under a nitrogen atmosphere was added wittig reagent [3-(tert-butyl-dimethyl-silanyloxy)-5-oxo-6-(triphenyl-phosphanylidene)-hexanoic acid methyl ester) (0.81 g, 1.5 mmol). The mixture was heated at reflux for 40 h and then concentrated in vacuo to give a residue, which was purified by chromatography (10%-50% ethyl acetate in hexanes) to give 0.52 g (66%) of the desired product as an yellow foam: mp 108-110° C.; MS(APCI⁺): m/z 780.4 (MH⁺); Anal. Calcd for C₄₀H₄₇F₂N₃O₇S₁Si₁: C, 61.60; H, 6.07; N, 5.39. Found: C, 61.42; H, 6.01; N, 5.46.

Step H

### (3R)-7-[3,4-Bis-(4-fluoro-phenyl)-1-isopropyl-5-(4-sulfamoyl-phenylcarbamoyl)-1H-pyrrol-2-yl]-3-hydroxy-5-oxo-hept-6-enoic Acid Methyl Ester

To a solution of (3R)-7-[3,4-bis-(4-fluoro-phenyl)-1-isopropyl-5-(4-sulfamoyl-phenylcarbamoyl)-1H-pyrrol-2-yl]-3-(tert-butyl-dimethyl-silanyloxy)-5-oxo-hept-6-enoic acid methyl ester prepared from step G (510 mg, 0.654 mmol) in acetonitrile (1 mL) was added dropwise a hydrogen fluoride solution (1:19 48% HF:acetonitrile, 4 mL) in an ice bath under a nitrogen atmosphere. The mixture was stirred at room temperature for 1 h. TLC showed that the reaction was complete. The mixture was partitioned between ethyl acetate and water. The organic phase was separated and washed with NaHCO₃ and brine, dried over Na₂SO₄ and filtered. The filtrate was concentrated in vacuo to give 430 mg (99%) of the desired product as a light yellow foam: mp 109-110° C.; MS(APCI⁺): m/z 666.3 (MH⁺); Anal. Calcd for C₃₄H₃₃F₂N₃O₇O₁.0.2CH₂Cl₂: C, 59.99; H, 4.90; N, 6.02. Found: C, 60.17; H, 4.93; N, 6.16.

Step I

### (3R,5S)-7-[3,4-Bis-(4-fluoro-phenyl)-1-isopropyl-5-(4-sulfamoyl-phenylcarbamoyl)-1H-pyrrol-2-yl]-3,5-dihydroxy-hept-6-enoic Acid Methyl Ester

To a mixture of (3R)-7-[3,4-bis-(4-fluoro-phenyl)-1-isopropyl-5-(4-sulfamoyl-phenylcarbamoyl)-1H-pyrrol-2-yl]-3-hydroxy-5-oxo-hept-6-enoic acid methyl ester prepared from step H (464 mg, 0.70 mmol) in THF (8 mL) and methanol (2 mL) was added dropwise a solution of 1M diethyl-methoxy-borane in THF (0.70 mL) at −78° C. under a nitrogen atmosphere. The mixture was stirred for 0.5 h and then sodium borohydride (26.4 mg, 0.70 mmol) was added in portions. After stirring for 2 h, 2 drops of acetic acid was added. The mixture was partitioned between ethyl acetate and water. The organic phase was separated and washed with NaHCO₃ and brine, dried over Na₂SO₄ and filtered. The filtrate was concentrated in vacuo to give a residue, which was dissolved in warm methanol and concentrated in vacuo again to give a residue, which was purified by chromatography (20%-75% ethyl acetate in hexanes) to give 270 mg (58%) of the desired product as a white foam: mp 208-210° C.; MS(APCI⁻): m/z 666.3 (M−H); Anal. Calcd for C₃₄H₃₅F₂N₃O₇S₁: C, 61.16; H, 5.28; N, 6.29. Found: C, 61.05; H, 5.16; N, 6.13.

Step J

### (3R,5R)-7-[3,4-Bis-(4-fluoro-phenyl)-1-isopropyl-5-(4-sulfamoyl-phenylcarbamoyl)-1H-pyrrol-2-yl]-3,5-dihydroxy-heptanoic Acid Methyl Ester

To a solution of (3R,5R)-7-[3,4-bis-(4-fluoro-phenyl)-1-isopropyl-5-(4-sulfamoyl-phenylcarbamoyl)-1H-pyrrol-2-yl]-3,5-dihydroxy-hept-6-enoic acid methyl ester prepared from step I (170 mg, 0.25 mmol) in THF (10 mL) and ethanol (10 mL) was added 10% palladium on activated carbon (50 mg). The mixture was stirred at room temperature under a hydrogen atmosphere for 3 h. TLC showed that the reaction was complete. The mixture was filtered through celite. The filtrate was concentrated in vacuo to give a residue, which was purified by chromatography (20-75% ethyl acetate in hexanes) to give 170 mg (100%) white solid: mp 106-108° C.; MS(APCI⁻): m/z 668.3 (M−H); Anal. Calcd for C₃₄H₃₇F₂N₃O₇S₁.0.3EtOAc: C, 60.73; H, 5.70; N, 6.04. Found: C, 60.42; H, 5.69; N, 5.68.

Step K

### (3R,5R)-7-[3,4-Bis-(4-fluoro-phenyl)-1-isopropyl-5-(4-sulfamoyl-phenylcarbamoyl)-1H-pyrrol-2-yl]-3,5-dihydroxy-heptanoic Acid Sodium Salt

To a mixture of (3R,5R)-7-[3,4-bis-(4-fluoro-phenyl)-1-isopropyl-5-(4-sulfamoyl-phenylcarbamoyl)-1H-pyrrol-2-yl]-3,5-dihydroxy-heptanoic acid methyl ester prepared from step J (177 mg, 0.264 mmol) in a solution of absolute ethanol (4 mL) and water (1 mL) was added 1N aqueous sodium hydroxide solution (0.264 mL) at room temperature. The mixture was stirred for 1 h and then concentrated in vacuo to give a residue, which was dissolved in a solution of 20% methanol in methylene chloride and filtered. The filtrate was concentrated in vacuo to give a solid. The solid was triturated with diethyl ether and filtered and dried in vacuo to give 179 mg (100%) of the desired product as a white solid: mp 261-263° C.; MS(APCI⁻): m/z 654.3 (M−H); Anal. Calcd for C₃₃H₃₄F₂N₃O₇S₁Na₁.2.0H₂O: C, 55.53; H, 5.37; N, 5.89. Found: C, 55.74; H, 5.48; N, 5.67.

### Example 12

### (3R,5S)-7-[3,4-Bis-(4-fluoro-phenyl)-1-isopropyl-5-(4-sulfamoyl-phenycarbamoyl-1H-pyrrol-2-yl]-3,5-dihydroxy-hept-6-enoic Acid Sodium Salt

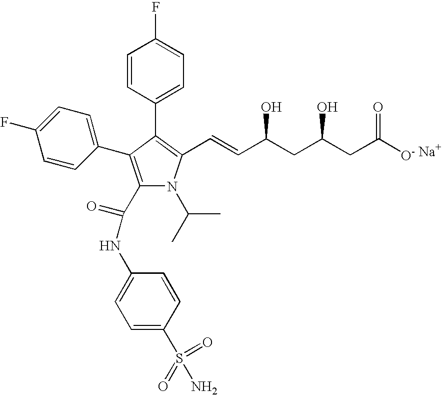

To a mixture of (3R,5R)-7-[3,4-bis-(4-fluoro-phenyl)-1-isopropyl-5-(4-sulfamoyl-phenylcarbamoyl)-1H-pyrrol-2-yl]-3,5-dihydroxy-hept-6-enoic acid methyl ester prepared from Example 12, Step I (50.2 mg, 0.075 mmol) in a solution of absolute ethanol (0.5 mL) and water (0.5 mL) was added 1N aqueous sodium hydroxide solution (0.075 mL) at room temperature. The mixture was stirred for 1 h and then concentrated in vacuo to give a residue, which was dissolved in a solution of 10% methanol in methylene chloride and filtered. The filtrate was concentrated in vacuo to give a solid. The solid was triturated with diethyl ether and filtered and dried in vacuo to give 50 mg (98%) of the desired product as an off-white solid: mp 267-269° C.; MS(APCI⁻): m/z 653.3 (M−H); Anal. Calcd for C₃₃H₃₂F₂N₃O₇S₁Na₁.3.5H₂O: C, 53.65; H, 5.32; N, 5.69. Found: C, 53.83; H, 5.05; N, 5.46.

### Example 13

### (3R,5R)-7-[5-(4-Carbamoylmethyl-phenylcarbamoyl)-3,4-bis-(4-fluoro-phenyl)-1-isopropyl-)-1H-pyrrol-2-yl]-3,5-dihydroxy-heptanoic Acid Sodium Salt

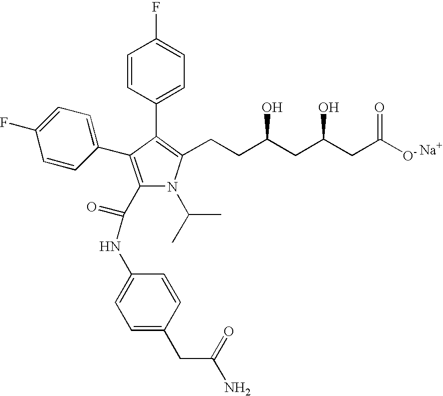

Example 13 was made by a method analogous to Example 11. mp 248-250° C.; MS(APCI⁻): m/z 632.3 (M−H); Anal. Calcd for C₃₅H₃₆F₂N₃O₆Na₁.2.5H₂O.0.05CH₂Cl₂: C, 59.72; H, 5.88; N, 5.96. Found: C, 59.83; H, 5.49; N, 5.60.

### Example 14

### (3R,5S)-7-[5-(4-Carbamoylmethyl-phenylcarbamoyl)-3,4-bis-(4-fluoro-phenyl)-1-isopropyl-1H-pyrrol-2-yl]-3,5-dihydroxy-hept-6-enoic Acid Sodium Salt

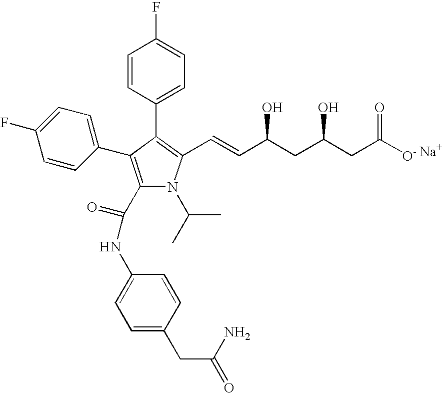

To a mixture of (3R,5R)-7-[5-(4-carbamoylmethyl-phenylcarbamoyl)-3,4-bis-(4-fluoro-phenyl)-1-isopropyl-1H-pyrrol-2-yl]-3,5-dihydroxy-hept-6-enoic acid methyl ester prepared from Example 13, Step D (30 mg, 0.046 mmol) in a solution of absolute ethanol (1 mL) and water (0.5 mL) was added 1N aqueous sodium hydroxide solution (0.046 mL) at room temperature. The mixture was stirred for 1 h and then concentrated in vacuo to give a residue, which was dissolved in a solution of 10% methanol in methylene chloride and filtered. The filtrate was concentrated in vacuo to give a solid. The solid was triturated with diethyl ether and filtered and dried in vacuo to give 30 mg (99%) of the desired product as a light yellow solid: mp 220-222° C.; MS(APCI⁻): m/z 631.3 (M−H); Anal. Calcd for C₃₅H₃₄F₂N₃O₆Na₁.2.5H₂O.0.15CH₂Cl₂: C, 59.34; H, 5.57; N, 5.91. Found: C, 59.41; H, 5.18; N, 5.74.

### Example 15

### (3R,5R)-7-[3,4-bis(4-fluorophenyl)-1-isopropyl-5-phenylcarbamoyl-1H-pyrrol-2-yl]-3,5-dihydroxy-heptanoic Acid Sodium Salt

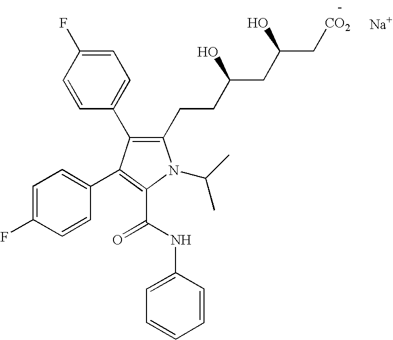

Example 15 was made by a method analogous to Example 11. MS(APCI⁺): m/z 577.3 (M+1); Anal. Calcd for C₃₃H₃₃F₂N₂O₅Na₁.1.06 CH₂Cl₂: C, 59.40; H, 5.14; N, 4.07. Found: C, 59.01; H, 5.39; N, 3.98.

### Example 16

### (3R,5R)-7-[3,4-bis(4-fluorophenyl)-5-(2-fluoro-phenylcarbamoyl)-1-isopropyl-1H-pyrrol-2-yl]-3,5-dihydroxy-heptanoic Acid Sodium Salt

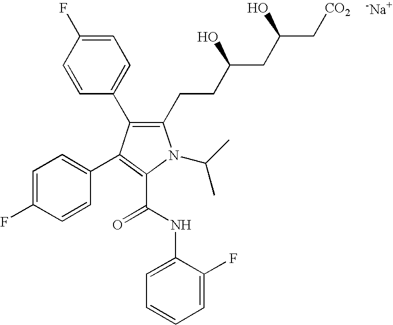

Example 16 was made by a method analogous to Example 11. MS(APCI⁺): m/z 595.2 (M+1); Anal. Calcd for C₃₃H₃₂F₃N₂O₅Na₁.0.73 CH₂Cl₂: C, 59.42; H, 4.95,; N, 4.10. Found: C, 59.05; H, 4.75; N., 4.04.

### Example 17

### (3R,5S)-7-[3,4-bis(4-fluorophenyl)-5-(4-fluorophenylcarbamoyl)-1-isopropyl-1H-pyrrol-2-yl]-3,5-dihydroxy-hept-6-enoic Acid Sodium Salt

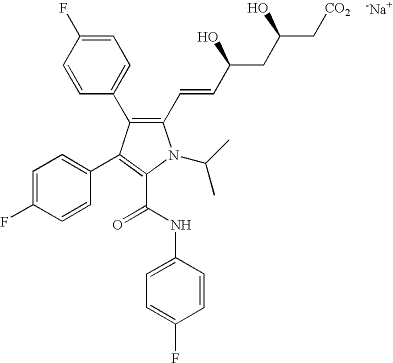

Example 17 was made by a method analogous to Example 12. MS(APCI⁺): m/z 593.2 (M+1); Anal. Calcd for C₃₃H₃₂F₃N₂O₅Na₁.3.73 NaOH: C, 51.70; H, 4.70; N, 4.65. Found: C, 51.33; H, 4.58; N, 3.38.

### Example 18

### (3R,5R)-7-[5-(2,4-difluoro-phenylcarbamoyl)-3,4-bis(4-fluoro-phenyl)-1-isopropyl-1H-pyrrol-2-yl]-3,5-dihydroxy-heptanoic Acid Sodium Salt

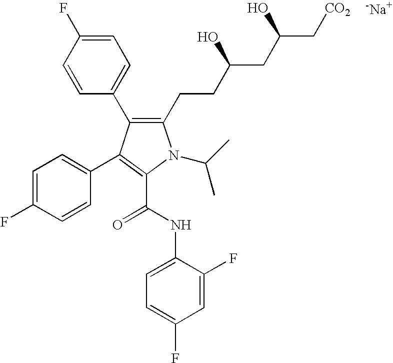

Example 18 was made by a method analogous to Example 12. MS(APCI⁺): m/z 613.1. (M+1); Anal. Calcd for C₃₃H₃₁F₄N₂O₅Na.1.00H₂O.0.35 CH₂Cl₂:C, 58.70; H, 4.98; N 4.11. Found: C, 58.32; H, 4.60; N, 3.72.

### Example 19

### (3R,5R)-7-[3,4-bis-(4-fluorophenyl)-1-isopropyl-5-p-tolylcarbamoyl-1H-pyrrol-2-yl]-3,5-dihydroxy-heptanoic Acid Sodium Salt

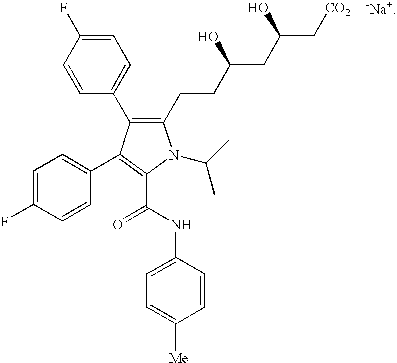

Example 19 was made by a method analogous to Example 11. MS(APCI⁺): m/z 591.2. (M+1); Anal. Calcd for C₃₄H₃₅F₂N₂O₅Na.0.35 CH₂Cl₂: C, 63.32; H, 5.54; N, 4.28. Found: C, 62.95; H, 5.90; N, 4.22.

### Example 20

### (3R,5R)-7-[3,4-bis(4-fluorophenyl)-1-isopropyl-5-m-tolylcarbamoyl-1H-pyrrol-2-yl]-3,5-dihydroxy-heptanoic Acid Sodium Salt

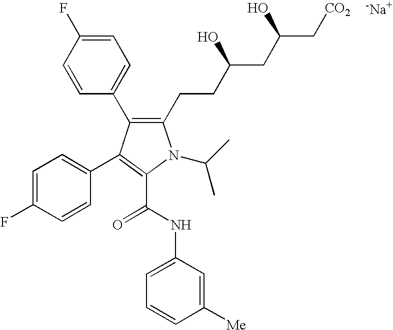

Example 20 was made by a method analogous to Example 11. MS(APCI⁺): m/z 591.2. Anal. Calcd for C₃₄H₃₅F₂N₂O₅Na.0.91CH₂Cl₂: C, 60.77; H, 5.38; N, 4.06. Found: C, 60.43; H, 5.50; N, 3.86.

### Example 21

### (3R,5R)-7-[1-Ethyl-3-(4-fluoro-phenyl)-4-isopropyl-5-phenylcarbamoyl-1H-pyrrol-2-yl]-3,5-dihydroxy-heptanoic Acid Sodium Salt

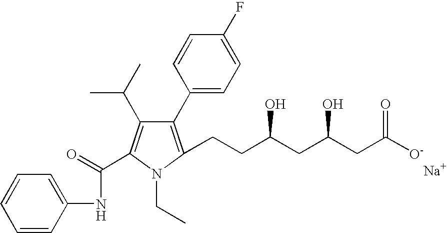 Step A

### 1-(4-Fluoro-phenyl)-3-methyl-1-nitro-butan-2-ol

A solution of 1-fluoro-4-nitromethyl-benzene (5.1 g, 33.0 mmol) and 2-methyl-propionaldehyde (3.0 ml, 2.4 g, 33.0 mmol) in 25.0 ml of tetrahydrofuran was treated with 1.3 g (˜3.4 mmol of base) of polymer-bound 1,5,7-triazabicyclo(4.4.0)dec-5-ene. The new mixture was stirred at room temperature for 18 h. The mixture was filtered, and the insoluble material was washed on the funnel with ethyl acetate. The combined filtrates were evaporated, and the residue was purified by chromatography (15% ethyl acetate in hexane) to give 4.0 g (54%) of the desired product as a clear oil, which slowly crystallized to a waxy solid: MS(APCI⁻): m/z 226 (M−H); Anal. Calcd for C₁₁H₁₄F₁N₁O₃: C, 58.14; H, 6.21; N, 6.16. Found: C, 58.06; H, 6.15; N, 6.01.

Step B

### 1-Fluoro-4-(3-methyl-1-nitro-but-1-enyl)-benzene

An ice cooled solution of 1-(4-fluoro-phenyl)-3-methyl-1-nitro-butan-2-ol (3.9 g, 17.0 mmol) prepared in step A in 50 ml of dichloromethane was treated dropwise via syringe with methanesulfonyl chloride (1.3 ml, 1.92 g, 16.8 mmol), followed by triethylamine (9.4 ml, 6.8 g, 67.4 mmol). The new mixture was stirred with continued ice cooling for 4 h. The bulk of the solvent was evaporated, and the residue was partitioned between ethyl acetate (200 ml) and brine (150 ml). The layers were separated, and the aqueous layer was extracted with fresh ethyl acetate (2×100 ml). The combined organic layers were washed with brine (2×200 ml), dried (Na₂SO₄) and evaporated. The residue was purified by chromatography (3% ethyl acetate in hexane) to give 1.0 g (29%) of the desired product as an oil: MS(APCI⁻): m/z 208 (M−H); 99% pure by HPLC.

Step C

### 4-(4-Fluoro-phenyl)-3-isopropyl-1H-pyrrole-2-carboxylic Acid Ethyl Ester

An ice cooled solution of 1-fluoro-4-(3-methyl-1-nitro-but-1-enyl)-benzene (2.69 g, 23.8 mmol) prepared in step B and ethyl isocyanoacetate (4.8 ml, 5.0 g, 23.7 mmol) in 30 ml of tetrahydrofuran and 30 ml of 2-propanol was treated dropwise via syringe with 1,1,3,3-tetramethylguanidine (3.1 ml, 2.85 g, 24.7 mmol). The mixture was stirred as the ice bath slowly melted for 16 h. The reaction mixture was condensed 75% on the rotary evaporator, and the residue was added to 300 g of ice and water. The new mixture was acidified with 4.0 N hydrochloric acid. The gummy, tan precipitate that formed was extracted with ethyl acetate (4×100 ml). The combined organic layers were washed with brine (2×200 ml), dried (Na₂SO₄) and evaporated. The residue was purified by chromatography (15% ethyl acetate in hexane) to give 4.6 g (73%) of the desired product as a yellow oil, which quickly crystallized to a solid: mp 94-97° C.; MS(APCI⁻): m/z 274 (M−H); Anal. Calcd for C₁₆H₁₈F₁N₁O₂: C, 69.80; H, 6.59; N, 5.09. Found: C, 69.52; H, 6.59; N, 5.10.

Step D

### 1-Ethyl-4-(4-fluoro-phenyl)-3-isopropyl-1H-pyrrole-2-carboxylic Acid Ethyl Ester

A mixture of 4-(4-fluoro-phenyl)-3-isopropyl-1H-pyrrole-2-carboxylic acid ethyl ester (5.1 g, 19.0 mmol) prepared in step C and iodoethane (5.0 ml, 9.8 g, 62.5 mmol) in 125 ml of acetonitrile was treated with cesium carbonate (9.1 g, 28.0 mmol). The mixture was stirred at room temperature for 68 h. The reaction mixture was filtered, and the insoluble material was washed several times on the funnel with fresh acetonitrile. The bulk of the solvent was evaporated, and the residue was partitioned between ethyl acetate (200 ml) and brine (150 ml). The layers were separated, and the aqueous layer was extracted with fresh ethyl acetate (2×100 ml). The combined organic layers were washed with brine (2×150 ml), dried (Na₂SO₄) and evaporated. The residue was purified by chromatography (7.5% ethyl acetate in hexane) to give 4.6 g (82%) of the desired product as a yellow solid: mp 74-76° C.; MS(APCI⁺): m/z 304 (MH⁺); Anal. Calcd for C₁₈H₂₂F₁N₁O₂: C, 71.26; H, 7.31; N, 4.62. Found: C, 71.31; H, 7.43; N, 4.65.

Step E

### 1-Ethyl-4-(4-fluoro-phenyl)-5-formyl-3-isopropyl-1H-pyrrole-2-carboxylic Acid Ethyl Ester

N,N-dimethylformamide (17.2 ml, 16.2 g, 222 mmol) was cooled in ice and treated dropwise via syringe with phosphorus oxychloride (6.9 ml, 11.4 g, 74.0 mmol). The mixture was stirred for 1 h with ice cooling, and a solution of 1-ethyl-4-(4-fluoro-phenyl)-3-isopropyl-1H-pyrrole-2-carboxylic acid ethyl ester (4.5 g, 15.0 mmol) prepared in step D in 60 ml of 1,2-dichloroethane was added dropwise. The cooling bath was removed, and the mixture was heated at reflux for 5 h. The reaction mixture was added to 250 ml of 5% aqueous sodium bicarbonate solution plus ice. To the mixture was added 150 ml of dichloromethane, and the new mixture was stirred at room temperature for 16 h. The pH of the reaction mixture was still strongly acidic. The mixture was cooled in ice, and solid sodium bicarbonate was added in portions until foaming had ceased and the pH was 7-8. The liquid was decanted from some insoluble material (inorganic) and added to a separatory funnel. The layers were separated, and the aqueous layer was extracted with fresh dichloromethane (3×150 ml). The residual solid (above) was washed on a filter funnel with several portions of fresh dichloromethane and the washes were added to the larger dichloromethane extracts. The combined organic layers were washed with brine (2×300 ml). The org. layer was dried (Na₂SO₄) and evaporated. The residue was purified by chromatography (5% ethyl acetate in hexane) to give 4.7 g (97%) of the desired product as an orange oil: MS(APCI⁺): m/z 332 (MH⁺); Anal. Calcd for C₁₉H₂₂F₁N₁O₃: C, 68.86; H, 6.69; N, 4.23. Found: C, 68.72; H, 6.51; N, 4.19.

Step F

### 1-Ethyl-4-(4-fluoro-phenyl)-5-formyl-3-isopropyl-1H-pyrrole-2-carboxylic Acid

A solution of 1-ethyl-4-(4-fluoro-phenyl)-5-formyl-3-isopropyl-1H-pyrrole-2-carboxylic acid ethyl ester (4.6 g, 13.9 mmol) prepared in step E in 75 ml of tetrahydrofuran was treated with lithium hydroxide monohydrate (2.0 g, 47.7 mmol), followed by 25 ml of water, and the mixture was stirred at reflux for 3 h. Thin layer chromatography indicated only partial saponification. An additional 2.0 g (47.7 mmol) of lithium hydroxide monohydrate and 25 ml of water were added, and the mixture was again heated at reflux for a total of 44 h. Approximately 50% of the reaction solvent was evaporated, and the residue was added to 400 g of ice and water. The solution was cooled in an ice bath and acidified with 4.0 N hydrochloric acid. The yellow precipitated product was extracted with ethyl acetate (4×150 ml). The combined organic layers were washed with brine (2×300 ml), dried (Na₂SO₄), and evaporated to give 4.0 g (95%) of the desired product as a yellow solid. A sample recrystallized from hexane/ethyl acetate had mp 182° C.-dec.; MS(APCI⁻): m/z 302 (M−H); Anal. Calcd for C₁₇H₁₈F₁N₁O₃: C, 67.31; H, 5.98; N, 4.62. Found: C, 67.31; H, 5.99; N, 4.51.

Step G

### 1-Ethyl-4-(4-fluoro-phenyl)-5-formyl-3-isopropyl-1H-pyrrole-2-carboxylic Acid Phenylamide

A solution of 1-ethyl-4-(4-fluoro-phenyl)-5-formyl-3-isopropyl-1H-pyrrole-2-carboxylic acid (3.46 g, 11.4 mmol) prepared in step F in 100 ml of dichloromethane was cooled in an ice bath and treated with 5 drops of N,N-dimethylformamide. A solution of oxalyl chloride (1.6 ml, 2.33 g, 18.3 mmol) in 20 ml of dichloromethane was added dropwise. The mixture was stirred as the ice bath slowly melted for 18 h. The mixture was evaporated to give the acid chloride product 1-ethyl-4-(4-fluoro-phenyl)-5-formyl-3-isopropyl-1H-pyrrole-2-carbonyl chloride as a dark red solid. The crude acid chloride was dissolved in dichloromethane (100 ml) and added dropwise to an ice cooled solution of aniline (1.2 ml, 1.23 g, 13.2 mmol) and N,N-diisopropylethylamine (2.4 ml, 1.78 g, 13.8 mmol) in dichloromethane (75 ml). The mixture was stirred as the ice bath slowly melted for 24 hr. The reaction mixture was added to 350 ml of brine. An additional 150 ml of dichloromethane was added, and the layers were separated. The aqueous layer was extracted with fresh dichloromethane (2×150 ml). The combined organic layers were washed with 2.0 N hydrochloric acid (3×300 ml), 5% aqueous sodium bicarbonate solution (3×300 ml), and brine (1×300 ml). The organic layer was dried (Na₂SO₄), and evaporated. The residue was purified by chromatography (20% ethyl acetate in hexane) to give 1.9 g (44%) of the desired product as a tan solid: MS(APCI⁺): m/z 379 (MH⁺); 98% pure by HPLC.

Step H

### (3R)-3-(tert-Butyl-dimethyl-silanyloxy)-7-[1-ethyl-3-(4-fluoro-phenyl)-4-isopropyl-5-phenylcarbamoyl-1H-pyrrol-2-yl]-5-oxo-hept-6-enoic Acid Methyl Ester

A solution of 1-ethyl-4-(4-fluoro-phenyl)-5-formyl-3-isopropyl-1H-pyrrole-2-carboxylic acid phenylamide (0.74 g, 1.96 mmol) prepared in step G and the Wittig reagent [3-(tert-butyl-dimethyl-silanyloxy)-5-oxo-6-(triphenyl-phosphanylidene)-hexanoic acid methyl ester] (2.1 g, 3.9 mmol) in 30 ml of toluene was stirred at reflux for 70 hr. The solvent was evaporated, and the residue was purified by chromatography (20% ethyl acetate in hexane) to give 0.64 g (52%) of the desired product as an orange solid: mp 143-1450C; MS(APCI⁺): m/z 635 (MH⁺); Anal. Calcd for C₃₆H₄₇F₁N₂O₅Si₁: C, 68.11; H, 7.46; N, 4.41. Found: C, 68.03; H, 7.46; N, 4.23.

Step I

### (3R)-7-[1-Ethyl-3-(4-fluoro-phenyl)-4-isopropyl-5-phenylcarbamoyl-1H-pyrrol-2-yl]-3-hydroxy-5-oxo-hept-6-enoic Acid Methyl Ester

A solution of (3R)-3-(tert-butyl-dimethyl-silanyloxy)-7-[1-ethyl-3-(4-fluoro-phenyl)-4-isopropyl-5-phenylcarbamoyl-1H-pyrrol-2-yl]-5-oxo-hept-6-enoic acid methyl ester (2.21 g, 3.48 mmol) prepared in step H in 35 ml of acetonitrile was cooled in ice and treated dropwise via syringe with a solution of 48% aqueous hydrofluoric acid in 10 ml of acetonitrile. The ice bath was removed, and the mixture was stirred as it warmed to room temperature for 2 h. The reaction mixture was added to 200 ml of ice cold 5% aqueous sodium bicarbonate solution. The mixture was extracted with ethyl acetate (4×75 ml). The combined organic layers were washed with 5% aqueous sodium bicarbonate (2×200 ml) and brine (1×200 ml). The organic layer was dried (Na₂SO₄) and evaporated, and the residue was purified by chromatography (40-75% ethyl acetate in hexane) to give 1.37 g (76%) of the desired product as a yellow foam: MS(APCI⁺): m/z 521 (MH⁺); 95% pure by HPLC.

Step J

### (3R,5S)-7-[1-Ethyl-3-(4-fluoro-phenyl)-4-isopropyl-5-phenylcarbamoyl-1H-pyrrol-2-yl]-3,5-dihydroxy-hept-6-enoic Acid Methyl Ester

A solution of (3R)-7-[1-ethyl-3-(4-fluoro-phenyl)-4-isopropyl-5-phenylcarbamoyl-1H-pyrrol-2-yl]-3-hydroxy-5-oxo-hept-6-enoic acid methyl ester (1.34 g, 2.57 mmol) prepared in step I in 20 ml of tetrahydrofuran plus 5 ml of methanol was cooled in a dry ice/acetone bath and treated dropwise via syringe with 2.7 ml (2.7 mmol) a solution of 1.0 M diethylmethoxyborane in tetrahydrofuran. The mixture was stirred for 45 min, and solid sodium borohydride (0.10 g, 2.64 mmol) was added in one portion. The new mixture was stirred for an additional 3 h with dry ice cooling, then allowed to warm to 0° C. The mixture was treated with 1.5 ml (˜26 mmol) of glacial acetic acid and allowed to warm to room temperature. The total reaction mixture was diluted with 200 ml of ethyl acetate. The solution was washed with brine (1×100 ml), 5% aqueous sodium bicarbonate solution (3×100 ml) and brine (1×100 ml) again. The organic layer was dried (Na₂SO₄) and evaporated to a yellow solid residue. The residue was stirred for 18 h in 100 ml of methanol, and the methanol solution was evaporated. The residue was purified by chromatography (50-75% ethyl acetate in hexane) to give 1.05 g (78%) of the desired product as an off-white foam: MS(APCI⁺): m/z 523 (MH⁺); Anal. Calcd for C₃₀H₃₅F₁N₂O₅: C, 68.95; H, 6.75; N, 5.36. Found: C, 68.76; H, 6.66; N, 5.15.

Step K

### (3R,5R)-7-[1-Ethyl-3-(4-fluoro-phenyl)-4-isopropyl-5-phenylcarbamoyl-1H-pyrrol-2—yl]-3,5-dihydroxy-heptanoic Acid Methyl Ester

A solution of (3R,5R)-7-[1-ethyl-3-(4-fluoro-phenyl)-4-isopropyl-5-phenylcarbamoyl-1H-pyrrol-2-yl]-3,5-dihydroxy-hept-6-enoic acid methyl ester (0.74 g, 1.42 mmol) prepared in step J in 16 ml of methanol was hydrogenated over 0.15 g 10% Pd/C catalyst for 16 h at room temperature. The catalyst was removed by filtration, and the residue was purified by chromatography (50-75% ethyl acetate in hexane) to give 0.53 g (71%) of the desired product as a white foam: MS(APCI⁺): m/z 525 (MH⁺); Anal. Calcd for C₃₀H₃₇F₁N₂O₅: C, 68.68; H, 7.11; N, 5.34. Found: C, 68.41; H, 7.33; N, 5.23.

Step L

A solution of (3R,5R)-7-[1-ethyl-3-(4-fluoro-phenyl)-4-isopropyl-5-phenylcarbamoyl-1H-pyrrol-2-yl]-3,5-dihydroxy-heptanoic acid methyl ester (0.48 g, 0.92 mmol) in 12 ml of absolute ethanol was treated with 6 ml of water followed by 1.0 M sodium hydroxide solution (0.91 ml, 0.91 mmol). The mixture was stirred at room temperature for 2 h. The total reaction mixture was evaporated to a semi-solid residue. The residue was suspended in acetone and evaporated again three times. The new residue was dissolved in a solution of 20% methanol in dichloromethane until no further solid went into solution (˜50 ml). The mixture was filtered, and the filtrate was evaporated. The final residue was stirred in 50 ml of ethyl ether for two days. The solid was filtered and washed on the funnel several times with fresh ether to give 0.45 g (92%) of the desired product as a white solid: MS(APCI⁺): m/z 511 (MH⁺ for the parent acid); 100% pure by HPLC.

### Example 22

### (3R,5R)-7-[1-ethyl-3-(4-fluoro-phenyl)-4-methyl-5-phenylcarbamoyl-1-Hpyrrol-2-yl]-3,5-dihydroxy-heptanoic Acid Sodium Salt

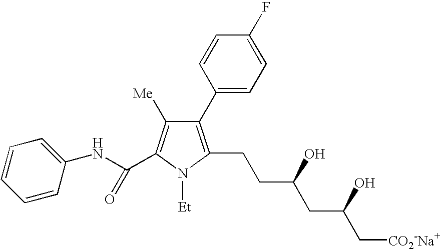 Step A

### 4-Bromo-3,5-dimethyl-1H-pyrrole-2-carboxylic Acid Ethyl Ester

A solution of 3,5-dimethyl-1H-pyrrole-2-carboxylic acid ethyl ester (22.6 g, 135 mmol) and pyridine (23.0 ml, 22.5 g, 284 mmol) in 350 ml of dichloromethane was cooled in an ice bath and treated dropwise with a solution of bromine (7.6 ml, 23.7 g, 148 mmol) in 100 ml of dichloromethane. The mixture was stirred in ice for 15 min after addition of the bromine was completed. The reaction mixture was added to 1000 ml of ice cold 2.0 N aqueous sodium thiosulfate solution in a separatory funnel. The layers were separated, and the aqueous layer was extracted with fresh dichloromethane (3×250 ml). The combined organic layers were washed with ice cold 2.0 N hydrochloric acid solution (3×500 ml), followed by 5% aqueous sodium bicarbonate solution (2×500 ml), and brine (1×500 ml). The combined organic layers were evaporated, and the residue was recrystallized from hexane to give 26.8 g (81%) of the desired product as white crystals: mp 140° C.-dec.; MS(APCI⁺): m/z 247 (MH⁺); Anal. Calcd for C₉H₁₂Br₁N₁O₂: C, 43.92; H, 4.91; N, 5.69. Found: C, 43.95; H, 4.83; N, 5.60.

Step B

### 4-(4-Fluoro-phenyl)-3,5-dimethyl-1H-pyrrole-2-carboxylic Acid Ethyl Ester

A solution of 4-bromo-3,5-dimethyl-1H-pyrrole-2-carboxylic acid ethyl ester (26.7 g, 108 mmol) prepared in step A and 4-fluorophenylboronic acid (22.0 g, 157 mmol) in 300 ml of N,N-dimethylformamide was treated with a solution of sodium carbonate (29.5 g, 278 mmol) dissolved in a minimum (˜80 ml) of water. The catalyst tetrakis(triphenylphosphine)palladium(0) (4.2 g, 3.6 mmol) was added, and the new mixture was stirred at reflux for 19 h. The reaction mixture was diluted with 1000 ml of ethyl acetate and filtered through a bed of Celite filter aid. The filtrate was washed with 5% aqueous sodium carbonate solution (3×1000 ml) and brine (3×1000 ml). The organic layer was dried (Na₂SO₄) and evaporated, and the residue was recrystallized from aqueous acetonitrile to give 20.2 g (71%) of the desired product as tan crystals: mp 174-175° C.; MS(APCI⁺): m/z 262 (MH⁺); Anal. Calcd for C₁₅H₁₆F₁N₁O₂: C, 68.95; H, 6.17; N, 5.36. Found: C, 68.79; H, 6.13; N, 5.30.

Step C

### 4-(4-Fluoro-phenyl)-5-formyl-3-dimethyl-1H-pyrrole-2-carboxylic Acid Ethyl Ester

A solution of 4-(4-fluoro-phenyl)-3,5-dimethyl-1H-pyrrole-2-carboxylic acid ethyl ester (10.1 g, 38.7 mmol) prepared in step B in 200 ml of tetrahydrofuran and 45 ml of acetic acid was treated with 100 ml of water. The two phase mixture was then treated with additional tetrahydrofuran (˜100 ml) until it again became one phase. Ceric ammonium nitrate (85.0 g, 155 mmol) was added in portions over a few minutes, and the mixture was stirred at room temperature for 2 h. The reaction mixture was poured into 1.0 kg of ice and water. The new mixture was extracted with dichloromethane (4×300 ml). The combined organic layers were washed with brine (2×500 ml), 5% aqueous sodium bicarbonate solution (4×500 ml), and brine (1×500 ml) again. The organic layer was dried (Na₂SO₄) and evaporated. The residue was purified by chromatography (20-30% ethyl acetate in hexane) to give 7.8 g (73%) of the desired product as a yellow solid: mp 144-146° C.; MS(APCI⁺): m/z 276 (MH⁺); Anal. Calcd for C₁₅H₁₄F₁N₁O₃: C, 65.45; H, 5.13; N, 5.09. Found: C, 65.28; H, 4.94; N, 4.92.

Step D

### 1-Ethyl-4-(4-fluoro-phenyl)-5-formyl-3-dimethyl-1H-pyrrole-2-carboxylic Acid Ethyl Ester

A suspension of 4-(4-fluoro-phenyl)-5-formyl-3-dimethyl-1H-pyrrole-2-carboxylic acid ethyl ester (6.6 g, 24.0 mmol) prepared in step C and iodo-ethane (7.5 ml, 14.6 g, 93.8 mmol) in 250 ml of acetonitrile was treated with cesium carbonate (11.8 g, 36.2 mmol). The mixture was stirred at room temperature for 21 h. The reaction mixture was filtered, and the insoluble material was washed several times on the funnel with fresh acetonitrile. The bulk of the solvent was evaporated, and the residue was partitioned between ethyl acetate (250 ml) and brine (200 ml). The layers were separated, and the aqueous layer was extracted with fresh ethyl acetate (2×100 ml). The combined organic layers were washed with brine (2×250 ml), dried (Na₂SO₄) and evaporated. The residue was purified by chromatography (10% ethyl acetate in hexane) to give 6.3g (87%) of the desired product as a syrup which rapidly crystallized to a solid: mp 69-71° C.; MS(APCI⁺): m/z 304 (MH⁺); Anal. Calcd for C₁₇H₁₈F₁N₁O₃: C, 67.31; H, 5.98; N, 4.62. Found: C, 67.30; H, 5.97; N, 4.55.

Step E

### 1-Ethyl-4-(4-fluoro-phenyl)-5-formyl-3-dimethyl-1H-pyrrole-2-carboxylic Acid

A solution of 1-ethyl-4-(4-fluoro-phenyl)-5-formyl-3-dimethyl-1H-pyrrole-2-carboxylic acid ethyl ester (6.6 g, 22.0 mmol) prepared in step D in 125 ml of tetrahydrofuran was treated with lithium hydroxide monohydrate (5.0 g, 119 mmol), followed by 50 ml of water, and the mixture was stirred at reflux for 22 h. Approximately 50% of the reaction solvent was evaporated, and the residue was added to 600 g of ice and water. The solution was cooled in an ice bath and acidified with 4.0 N hydrochloric acid. The yellow precipitated product was extracted with ethyl acetate (4×250 ml). The combined organic layers were washed with brine (2×500 ml), dried (Na₂SO₄), and evaporated to give 5.9 g (99%) of the desired product as an orange solid. A sample recrystallized from hexane/ethyl acetate had mp 213-2150C; MS(APCI⁻): m/z 274 (M−H); Anal. Calcd for C₁₅H₁₄F₁N₁O₃: C, 65.45; H, 5.13; N, 5.09. Found: C, 65.34; H, 5.11; N, 5.00.

The remaining steps are similar to steps G-L of Example 21.

### Example 23

### (3R,5R)-7-[3,4-bis(4-fluorophenyl)-1-isopropyl-5-(piperidine-1-carbonyl)-1H-pyrrol-2-yl]-3,5-dihydroxy-heptanoic Acid Sodium Salt

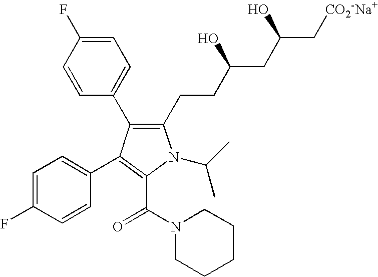 Step A

### 3,4-Bis(4-fluorophenyl)-1-isopropyl-5-(piperidine-1-carbonyl)-1H-pyrrole-2-carbaldehyde

3,4-Bis(4-fluorophenyl)-5-formyl-1-isopropyl-1H-pyrrole-2-carboxylic Acid was placed in Thionyl Chloride (5 mL) under nitrogen atmosphere and refluxed 1 h. The resulting mixture was concentrated under vacuum and the resulting solid dissolved in Ethyl Acetate (10 mL) and added dropwise to mixture of Piperidine (0.54 mL) and Sodium Carbonate (0,29 g) in Ethyl Acetate (40 mL) and water (7.0 mL) chilled in an ice-bath under nitrogen atmosphere. The reaction mixture was stirred 1 h at C, warmed to room temperature, and stirred 15h. The reaction mixture was poured into 2N HCL (100 mL) and extracted with Ethyl Acetate. The combined extracts were washed with water and brine and the organic phase dried over MgSO₄ and concentrated in vacuo. The residue was recyrstallized from EtAOc/Hexane to 0.77 g (65%) of a white solid: MS(APCI⁺): m/z 437.2 (M+H); Anal. Calcd for C₂₆H₂₆F₂N₂O₂: C, 71.54; H, 6.00; N, 6.42. Found: C, 71.28; H, 5.87; N, 6.26.

Step B

### (3R)-7-[3,4-Bis(4-fluorophenyl)-1-isopropyl-5-(piperidine-1-carbonyl)-1H-pyrrol-2-yl]-3-(tert-butyl-dimethyl-silanyloxy)-5-oxo-hept-6-enoic Acid Methyl Ester

To a mixture of (3R)-3,4-Bis(4-fluorophenyl)-1-isopropyl-5-(piperidine-1-carbonyl)-1H-pyrrole-2-carbaldehyde (0.78 g, 1.5 mmol) in Toluene (30 mL) at room temperature under a nitrogen atmosphere was added wittig reagent [3-(tert-butyl-dimethyl-silanyloxy)-5-oxo-6-(triphenyl-phosphanylidene)-hexanoic acid methyl ester] (1.2 g, 2.2 mmol). The mixture was heated at reflux for 64 h and then concentrated in vacuo to give a residue, which was purified by chromatography (5 to 50% EtOAc in Hexane) to give 0.68 g of a mixture of starting material and desired product. Used as is.

Step C

### (3R)-7-[3,4-Bis-(4-fluoro-phenyl)-1-isopropyl-5-(piperidine-1-carbonyl)-1H-pyrrol-2-yl]-3-hydroxy-5-oxo-hept-6-enoic Acid Methyl Ester

To a solution of the mixture (3R)-7-[3,4-Bis(4-fluorophenyl)-1-isopropyl-5-(piperidine-1-carbonyl)-1H-pyrrol-2-yl]-3-(tert-butyl-dimethyl-silanyloxy)-5-oxo-hept-6-enoic acid methyl ester and 3,4-Bis(4-fluorophenyl)-1-isopropyl-5-(piperidine-1-carbonyl)-1H-pyrrole-2-carbaldehyde (0.68 g) prepared from step B in acetonitrile (10 mL) was added dropwise a hydrogen fluoride solution (1:10 48% HF:acetonitrile, 3.0 mL) in an ice bath under a nitrogen atmosphere. The mixture was stirred at room temperature for 3h. TLC showed that the reaction was complete. The mixture was diluted with saturated aqueous NaHCO₃, partitioned between ethyl acetate and water. The organic phase was separated and washed with brine, dried over Na₂SO₄ and filtered. The filtrate was concentrated in vacuo and the residue purified by flash chromatography (5%-75% EtOAc/Hexane) to give 0.46g (81%) for both steps B and C; MS(APCI⁺): m/z 579.2 (M+H); NMR (CDCl₃) δ 0.87-0.88 (1H, m), 1.15-1.19 (1H, m), 1.22-1.40 (3H, m), 1.42-1.60 (3H, m)1.68 (6H, dd, J=31.7, J=6.8 Hz), 2.43-2.47(1H, m), 2.54 (1H, d, J=5.9 Hz), 2.78-3.82 (1H, m), 3.00-3.04 (1H, m), 3.38-3.42 (1H, m) 3.44-3.46 (1H, m), 3.58-3.63 (1H, m), 3.62 (3H, d, J=2.7), 4.38-4.42 (1H, m), 5.88 (2H, d, J=15.9 Hz), 6.94-7.08 (8H, m), 7.64 (1H, d, J=16 Hz).

Step D

### (3R,5S)-7-[3,4-Bis-(4-fluoro-phenyl)-1-isopropyl-5-(piperidine-1-carbonyl)-1H-pyrrol-2-yl]-3,5-dihydroxy-hept-6-enoic Acid Methyl Ester

To a mixture of (3R)-7-[3,4-Bis-(4-fluoro-phenyl)-1-isopropyl-5-(piperidine-1-carbonyl)-1H-pyrrol-2-yl]-3-hydroxy-5-oxo-hept-6-enoic acid methyl ester (0.44 g, 0.76 mmol), prepared from step D, in THF (10 mL) was added dropwise a solution of 1.1M Diethyl-methoxy-borane in THF (1.0 mL) at −78° C. under a nitrogen atmosphere. The mixture was stirred for 0.5 h and then Sodium Borohydride (38 mg, 1.0 mmol) was added in portions. After stirring for 2h, a few drops of acetic acid were added and the mixture was partitioned between ethyl acetate and water. The organic phase was separated and washed with NaHCO₃ and brine, dried over Na₂SO₄ and filtered. The filtrate was concentrated in vacuo to give a residue, which was dissolved in warm methanol and concentrated in vacuo again to give a residue, which was purified by flash chromatography (20%-100% ethyl acetate in hexanes) to give 0.22 g (50%) of the desired product as a white foam; MS(APCI⁺): m/z 580.2 (M+H); Anal. Calcd for C₃₃H₃₈F₂N₂O₅0.25EtOAc 0.20CH₂Cl₂: C, 66.29; H, 6.57; N, 4.52. Found: C, 66.55; H, 6.55; N, 4.13.

Step E

### (3R,5R)-7-[3,4-Bis-(4-fluoro-phenyl)-1-isopropyl-5-(piperidine-1-carbonyl)-1H-pyrrol-2-yl]-3,5-dihydroxy-heptanoic Acid Methyl Ester

To a solution of (3R,5R)-7-[3,4-Bis-(4-fluoro-phenyl)-1-isopropyl-5-(piperidine-1-carbonyl)-1H-pyrrol-2-yl]-3,5-dihydroxy-hept-6-enoic acid methyl ester (0.20 g, 0.34 mmol) in THF (10 mL) was added 10% Palladium on activated carbon (61 mg). This mixture was stirred at room temperature under hydrogen atmosphere for 3 h then filtered through celite. The filtrate was concentrated in vacuo to give a residue which was purified by flash chromatography (10%-100% EtOAc/Hexane) to give 156 mg (78%) of a white solid: MS(APCI⁻): m/z 582.2 (M+H); Anal. Calcd for C₃₃H₄₀F₂N₂O₅.0.12EtOAc: C, 67.78; H, 6.96; N, 4.72. Found: C, 67.39; H, 6.85; N, 4.63.

Step F

To a mixture of (3R,5R)-7-[3,4-Bis-(4-fluoro-phenyl)-1-isopropyl-5-(piperidine-1-carbonyl)-1H-pyrrol-2-yl]-3,5-dihydroxy-heptanoic acid methyl ester, prepared from step E, (63 mg, 0.10 mmol) in a solution of absolute ethanol (5.0 mL) was added 0.10 N aqueous sodium hydroxide solution (1.1 mL) at room temperature. The mixture was stirred for 1 h and then concentrated in vacuo to give a residue, which was dissolved in MeOH (2 mL) and Toluene (5 mL) then concentrated in vacuo to give a solid. This procedure was repeated and residue was triturated with dichloromethane, filtered, and dried in vacuo to give 59 mg (94%) of the desired product as a white solid: MS(APCI⁺): m/z 569.2. Anal. Calcd for C₃₂H₃₇F₂N₂O₅Na.2.28H₂O: C, 60.84; H, 6.63; N, 4.43. Found: C, 60.45; H, 6.25; N, 4.24.

### Example 24

### (4R,6R)-(6-{2-[3,4-Bis-(4-fluoro-phenyl)-1-isopropyl-1H-pyrrol-2-yl]-ethyl}-2,2-dimethyl-[1,3]dioxan-4-yl)-acetic Acid Methyl Ester

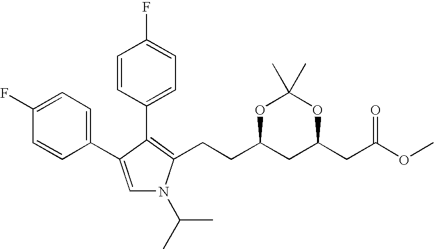 Step A

### 3,4-Bis-(4-fluoro-phenyl)-5-formyl-1-isopropyl-1H-pyrrole-2-carboxylic Acid Benzyl Ester

To a solution of 3,4-bis-(4-fluoro-phenyl)-5-formyl-1-isopropyl-1H-pyrrole-2-carboxylic acid prepared from example xx (0.75 g, 2.03 mmol) in THF (5 mL) was added DBU (0.364 mL, 2.47 mmol) followed by benzyl bromide (0.29 mL, 2.47 mmol) dropwise at room temperature under a nitrogen atmosphere. The mixture was stirred at room temperature overnight and partitioned between ethyl acetate and water. The organic phase was separated and washed with NaHCO₃ and brine, dried over Na₂SO₄ and filtered. The filtrate was concentrated in vacuo to give a residue, which was purified by chromatography (10% ethyl acetate in hexanes) to give 885 mg (95%) of the desired product as a white solid: mp 94-95° C.; MS(APCI⁺): m/z 460.2 (MH⁺); Anal. Calcd for C₂₈H₂₃F₂N₁O₃: C, 73.19; H, 5.05; N, 3.05. Found: C, 73.15; H, 4.95; N, 2.95.

Step B

### (5R)-5-[5-(tert-Butyl-dimethyl-silanyloxy)-6-methoxycarbonyl-3-oxo-hex-1-enyl]-3,4-bis-(4-fluoro-phenyl)-1-isopropyl-1H-pyrrole-2-carboxylic Acid Benzyl Ester

To a mixture of 3,4-bis-(4-fluoro-phenyl)-5-formyl-1-isopropyl-1H-pyrrole-2-carboxylic acid benzyl ester prepared from step A (0.98 g, 2.14 mmol) in toluene (10 mL) at room temperature under a nitrogen atmosphere was added wittig reagent [3-(tert-butyl-dimethyl-silanyloxy)-5-oxo-6-(triphenyl-phosphanylidene)-hexanoic acid methyl ester] (1.7 g, 3.21 mmol). The mixture was heated at reflux for 24 h and then concentrated in vacuo to give a residue, which was purified by chromatography (12% ethyl acetate in hexanes) to give 1.3 g (85%) of the desired product as a yellow syrup: MS(APCI⁺): m/z 716.3 (MH⁺); Anal. Calcd for C₄₁H₄₇F₂N₁O₆Si₁: C, 68.79; H, 6.62; N, 1.96. Found: C, 69.14; H, 6.47; N, 1.87.

Step C

### (5R)-3,4-Bis-(4-fluoro-phenyl)-5-(5-hydroxy-6-methoxycarbonyl-3-oxo-hex-1-enyl)-1-isopropyl-1H-pyrrole-2-carboxylic Acid Benzyl Ester

To a solution of (5R)-5-[5-(tert-butyl-dimethyl-silanyloxy)-6-methoxycarbonyl-3-oxo-hex-1-enyl]-3,4-bis-(4-fluoro-phenyl)-1-isopropyl-1H-pyrrole-2-carboxylic acid benzyl ester prepared from step B (1.25 g, 1.75 mmol) in acetonitrile (2 mL) was added dropwise a hydrogen fluoride solution (1:19 48% HF:acetonitrile, 8 mL) in an ice bath under a nitrogen atmosphere. The mixture was stirred at room temperature for 1 h. TLC showed that the reaction was complete. The mixture was partitioned between ethyl acetate and water. The organic phase was separated and washed with NaHCO₃ and brine, dried over Na₂SO₄ and filtered. The filtrate was concentrated in vacuo to give 1.04 g (100%) of the desired product as a light yellow foam: MS(APCI⁺): m/z 602.2 (MH⁺); Anal. Calcd for C₃₅H₃₃F₂N₁O₆.0.15EtOAc: C, 69.54; H, 5.61; N, 2.28. Found: C, 69.46; H, 5.42; N, 2.21.

Step D

### (3R,5R)-5-(3,5-Dihydroxy-6-methoxycarbonyl-hex-1-enyl)-3,4-bis-(4-fluoro-phenyl)-1-isopropyl-1H-pyrrole-2-carboxylic Acid Benzyl Ester

To a mixture of (5R)-3,4-bis-(4-fluoro-phenyl)-5-(5-hydroxy-6-methoxycarbonyl-3-oxo-hex-1-enyl)-1-isopropyl-1H-pyrrole-2-carboxylic acid benzyl ester prepared from step C (1.04 g, 1.73 mmol) in THF (8 mL) and methanol (2 mL) was added dropwise a solution of 1M diethyl-methoxy-borane in THF (1.73 mL) at −78° C. under a nitrogen atmosphere. The mixture was stirred for 0.5 h and then sodium borohydride (65 mg, 1.73 mmol) was added in portions. After stirring for 2 h, 3 drops of acetic acid were added. The mixture was partitioned between ethyl acetate and water. The organic phase was separated and washed with NaHCO₃ and brine, dried over Na₂SO₄ and filtered. The filtrate was concentrated in vacuo to give a residue, which was dissolved in warm methanol and concentrated in vacuo again to give a residue, which was purified by chromatography (20%-40% ethyl acetate in hexanes) to give 930 mg (89%) of the desired product as an off-white solid: mp 105-107° C.; MS(APCI⁺): m/z 604.3 (MH⁺); Anal. Calcd for C₃₅H₃₅F₂N₁O₆: C, 69.64; H, 5.84; N, 2.32. Found: C, 69.53; H, 5.89; N, 2.19.

Step E

### (4R,6R)-3,4-Bis-(4-fluoro-phenyl)-1-isopropyl-5-[2-(6-methoxycarbonylmethyl-2,2-dimethyl-[1,3]dioxan-4-yl)-vinyl]-1H-pyrrole-2-carboxylic Acid Benzyl Ester

To a solution of (3R,5R)-5-(3,5-dihydroxy-6-methoxycarbonyl-hex-1-enyl)-3,4-bis-(4-fluoro-phenyl)-1-isopropyl-1H-pyrrole-2-carboxylic acid benzyl ester prepared from step D (136 mg, 0.23 mmol) in acetone (5 mL) was added dimethoxypropane (0.04 mL, 0.34 mmol) followed by p-tolenesulphonic acid (5 mg). The mixture was stirred at room temperature for 1 h. TLC showed that the reaction was complete. The mixture was partitioned between ethyl acetate and water. The organic phase was separated and washed with NaHCO₃ and brine, dried over Na₂SO₄ and filtered. The filtrate was concentrated in vacuo to give a residue, which was purified by chromatography (5%-15% ethyl acetate in hexanes) to give 93 mg (64%) of the desired product as a white solid: mp 125-127° C.; MS(APCI⁺): m/z 644.3 (MH⁺); Anal. Calcd for C₃₈H₃₉F₂N₁O₆: C, 70.90; H, 6.11; N, 2.18. Found: C, 70.67; H, 6.03; N, 2.13.

Step F

### (4R,6R)-(6-{2-[3,4-Bis-(4-fluoro-phenyl)-1-isopropyl-1H-pyrrol-2-yl]-ethyl}-2,2-dimethyl-[1,3]dioxan-4-yl)-acetic Acid Methyl Ester

To a solution of (4R,6R)-3,4-bis-(4-fluoro-phenyl)-1-isopropyl-5-[2-(6-methoxycarbonylmethyl-2,2-dimethyl-[1,3]dioxan-4-yl)-vinyl]-1H-pyrrole-2-carboxylic acid benzyl ester prepared from step E (650 mg, 1.01 mmol) in THF (5 mL) and ethanol (15 mL) was added 10% palladium on activated carbon (100 mg). The mixture was stirred at room temperature under a hydrogen atmosphere for 3 h. TLC showed that the reaction was complete. The mixture was filtered through celite. The filtrate was concentrated in vacuo to give a residue, which was purified by chromatography (10%-50% ethyl acetate in hexanes) to give 501 mg (97%) of the desired product as a white solid: mp 55-57° C.; MS(APCI⁺): m/z 512.2 (MH⁺); Anal. Calcd for C₃₀H₃₅F₂N₁O₄: C, 70.43; H, 6.90; N, 2.74. Found: C, 70.12; H, 7.04; N, 2.67.

### Example 25

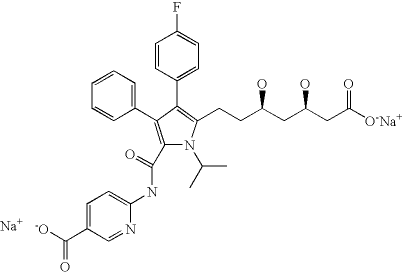

### 6-{[5-(6-Carboxy-3,5-dihydroxy-hexyl)-4-(4-fluoro-phenyl)-1-isopropyl-3-phenyl-1H-pyrrole-2-carbonyl]-amino}-nicotinic Acid di-sodium Salt

Step A

### 6-Iodo-nicotinic Acid

This compound was made according to the procedure published in Journal of Organic Chemistry, 1986, 51, 953-954.

Step B

### 6-Iodo-nicotinic Acid Methyl Ester

To a mixture of 6-Iodo-nicotinic acid (6.8 g, 27.4 mmol), toluene (40 mL) and MeOH (40 mL) cooled at 0° C., was added TMS diazomethane dropwise. The reaction mixture was then stirred at ambient temperature for additional 2 hours. The reaction mixture was concentrated in vacuo, the yellow residue was recrystallized from toluene to give the desired product as yellow crystals (5.6 g), MP 136-138° C., MS (APCI⁺): m/z 263.8 (M+H).

Step C

### (6-{2-[3-(4-Fluoro-phenyl)-1-isopropyl-4-phenyl-1H-pyrrol-2-yl]-ethyl}-2,2-dimethyl-[1,3]dioxan-4-yl)-acetic Acid Methyl Ester

This compound was made in a similar matter as shown for Example 24.

Step D

### (6-{2-[5-Carbamoyl-3-(4-fluoro-phenyl)-1-isopropyl-4-phenyl-1H-pyrrol-2-yl]-ethyl}-2,2-dimethyl-[1,3]dioxan-4-yl)-acetic Acid Methyl Ester

To a solution of (6-{2-[3-(4-fluoro-phenyl)-1-isopropyl-4-phenyl-1H-pyrrol-2-yl]-ethyl}-2,2-dimethyl-[1,3]dioxan-4-yl)-acetic acid methyl ester (3.06g, 6.2 mmol) in Et₂O (50 mL) was added chlorosulfonyl isocyanate (1.08 mL, 12.4 mmol) dropwise. The reaction mixture was stirred at ambient temperature for 40 minutes, saturated aqueous solution of NaHCO₃ (75 mL) was then added, the reaction mixture was stirred for another 5 minutes, white precipitate formed, the mixture was diluted with EtOAc, and the two phases were partitioned, organic phase was washed again with saturated aqueous solution of NaHCO₃, then mixed with MgSO₄ and stirred for 5 minutes. The solution was concentrated to give a white foam. The crude product was further purified by chromatography (1-60% EtOAc in hexanes) to give the desired product (2.22 g) as a white foam: MP 68-740C, MS (APCI⁺): m/z 537.2 (M+H).

Step E

### 6-({4-(4-Fluoro-phenyl)-1-isopropyl-5-[2-(6-methoxycarbonylmethyl-2,2-dimethyl-[1,3] dioxan-4-yl)-ethyl]-3-phenyl-1H-pyrrole-2-carbonyl}-amino)-nicotinic Acid Methyl Ester

(6-{2-[5-Carbamoyl-3-(4-fluoro-phenyl)-1-isopropyl-4-phenyl-1H-pyrrol-2-yl]-ethyl]-2,2-dimethyl-[1,3]dioxan-4-yl)-acetic acid methyl ester (2.2 g, 4.1 mmol), 6-iodo-nicotinic acid methyl ester (1.3 g, 4.9 mmol), N,N′-dimethylethylenediamine (0.089 mL, 0.82 mmol), copper iodide (0.078 g, 0.41 mmol), and potassium phosphate tribasic (1.8 g, 8.2 mmol) were mixed in a flask and 2.7 mL of dry DMF was added. The resulting mixture was stirred under nitrogen at 75° C. for 7 hours. The reaction mixture was then cooled to ambient temperature and diluted with EtOAc. The mixture was then washed with water (2×50 mL), dried over Na₂SO₄, and concentrated in vacuo. The residue was purified by chromatography (1-70% EtOAc in hexanes) to give the desired product (1.5 g) as a white foam: MP 77-84° C., MS (APCI⁺): m/z 672.2 (M+H).

Step F

### 6-{[5-(3,5-Dihydroxy-6-methoxycarbonyl-hexyl)-4-(4-fluoro-phenyl)-1-isopropyl-3-phenyl-1H-pyrrole-2-carbonyl]-amino}-nicotinic Acid Methyl Ester

To a solution of 6-({4-(4-fluoro-phenyl)-1-isopropyl-5-[2-(6-methoxycarbonylmethyl-2,2-dimethyl-[1,3]dioxan-4-yl)-ethyl]-3-phenyl-1H-pyrrole-2-carbonyl}-amino)-nicotinic acid methyl ester (1.5 g, 2.2 mmol) in MeOH (20 mL) was added 1 N HCl (2.2 mL), the resulting mixture was stirred for 18 hours. The reaction mixture was diluted with 150 mL of EtOAc, and them washed with water (2×60 mL) and brine (2×60 mL), dried over Na₂SO₄. The mixture was filtered and concentrated in vacuo. The residue was purified by chromatography (1-80% EtOAc in hexanes) to give the desired product (0.9355 g) as a white foam: MS (APCI⁺): m/z 632.2 (M+H), MP 71-75° C.

Step G

### 6-{[5-(6-Carboxy-3,5-dihydroxy-hexyl)-4-(4-fluoro-phenyl)-1-isopropyl-3-phenyl-1H-pyrrole-2-carbonyl]-amino}-nicotinic Acid

To a solution of 6-{[5-(3,5-dihydroxy-6-methoxycarbonyl-hexyl)-4-(4-fluoro-phenyl)-1-isopropyl-3-phenyl-1H-pyrrole-2-carbonyl]-amino}-nicotinic acid methyl ester (0.92 g, 1.5 mmol) in MeOH (30 mL) was added 1 N NaOH (7.3 mL), the resulting mixture was stirred at 60° C. for 1.0 hrs. After cooling to ambient temperature, 1N HCl aqueous solution (7.3 mL) was added to the reaction mixture; the reaction mixture was stripped to dryness. EtOH was added to dissolve the di-acid and the precipitate (NaCl) was removed by filtration. The filtrate was concentrated in vacuo to give the desired product as a white solid: MS (APCI⁺): m/z 604.2 (M+H).

Step H

### 6-{[5-(6-Carboxy-3,5-dihydroxy-hexyl)-4-(4-fluoro-phenyl)-1-isopropyl-3-phenyl-1H-pyrrole-2-carbonyl]-amino}-nicotinic Acid di-sodium Salt

To a solution of 6-{[5-(6-carboxy-3,5-dihydroxy-hexyl)-4-(4-fluoro-phenyl)-1-isopropyl-3-phenyl-1H-pyrrole-2-carbonyl]-amino}-nicotinic acid (1.1 g, 1.8 mmol) in MeOH (30 mL) was added 1 N NaOH aqueous solution (3.6 mL), the resulting mixture was stirred at ambient temperature for 1.0 hrs. The reaction mixture was stripped to dryness. The residue was dissolved in small amount of MeOH and mixed with toluene, the mixture was then concentrated in vacuo, this treatment was repeated three times to remove water. The residue was triturated with Et₂O to give the desired product (1.1 g) as a white solid: MS (APCI⁺): m/z 604.2 (M+H for the parent); MP >250° C.

### Example 26

### 7-[5-(Acetylamino-methyl)-3-(4-fluoro-phenyl)-1-isopropyl-4-phenyl-1H-pyrrol-2-yl]-3,5-dihydroxy-heptanoic Acid

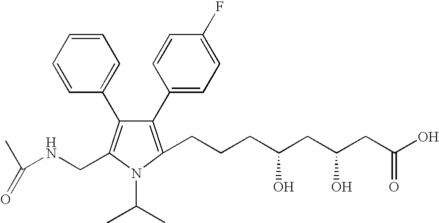

### Step A (6-{2-[3-(4-Fluoro-phenyl)-5-iodo-1-isopropyl-4-phenyl-1H-pyrrol-2-yl]-ethyl}-2,2-dimethyl-[1,3]dioxan-4-yl)-acetic Acid Methyl Ester

N-iodosuccinimide (1.35g) was added to (6-{2-[3-(4-Fluoro-phenyl)-1-isopropyl-4-phenyl-1H-pyrrol-2-yl]-ethyl)-2,2-dimethyl-[1,3]dioxan-4-yl)-acetic acid methyl ester (2.82g) in 15 mL of DMF, stirred at RT for 2 hours. After removal of the solvent, the residue was chromatographed on silica gel with AcOEt/hexanes as an eluent to afford (2.4g, 68%) as yellow form, MS m/z 620 (M+1), 400 MHz ¹H NMR (CDCl₃) δ 6.8-7.19 (m, 9H), 4.32 (m, 1H), 4.1 (br, 1H), 3.65 (br, 1H), 3.64 (s, 3H), 2.65 (m, 1H), 2.57 (m, 1H), 2.38 (abq, 2H), 1.62 (m, 1H), 1.43 (d, 6H), 1.38 (m, 1H), 1.31 (d, 6H).

### Step B (6-{2-[5-Cyano-3-(4-fluoro-phenyl)-1-isopropyl-4-phenyl-1H-pyrrol-2-yl]-ethyl)-2,2-dimethyl-[1,3]dioxan-4-yl)-acetic Acid Methyl Ester

A solution of 6-{2-[3-(4-Fluoro-phenyl)-5-iodo-1-isopropyl-4-phenyl-1H-pyrrol-2-yl]-ethyl)-2,2-dimethyl-[1,3]dioxan-4-yl)-acetic acid methyl ester (2.4 g), KCN (0.38 g) and Cu(CN) (0.45 g) in DMF (15 mL) was heated to 120° C. for 3 hours. After removal of the solvent, the residue was mixed with 100 mL of DCM and filtered the precipitated. After concentrated the DCM solution, the oil was chromatographed on silica gel with AcOEt/hexanes as an eluent to afford the 6-{2-[5-Cyano-3-(4-fluoro-phenyl)-1-isopropyl-4-phenyl-1H-pyrrol-2-yl]-ethyl)-2,2-dimethyl-[1,3]dioxan-4-yl)-acetic acid methyl ester (1.3g, 65%), MS m/z 519 (M+1), 400 MHz ¹H NMR (CDCl₃) δ 6.9-7.2 (m, 9H), 4.57 (m, 1H), 4.08 (m, 1H), 3.65 (br, 1H), 3.64 (s, 3H), 2.74 (br, 1H), 2.67 (m, 1H), 2.39 (abq, 2H), 1.69 (m, 6H), 1.62 (m, 1H), 1.42 (m, 1H), 1.31 (d, 6H).

### Step C (6-{2-[5-Aminomethyl-3-(4-fluoro-phenyl)-1-isopropyl-4-phenyl-1H-pyrrol-2-yl]-ethyl}-2,2-dimethyl-[1,3]dioxan-4-yl)-acetic Acid Methyl Ester

1 gram of (6-{2-[5-Cyano-3-(4-fluoro-phenyl)-1-isopropyl-4-phenyl-1H-pyrrol-2-yl]-ethyl}-2,2-dimethyl-[1,3]dioxan-4-yl)-acetic acid methyl ester was hydrogenated in MeOH over Raney Nickel (2.5g) at 100 psi pressure and RT for 60 h. The Ra—Ni was filtered and and the solvent removed, to afford an off-white solid 6-{2-[5-Aminomethyl-3-(4-fluoro-phenyl)-1-isopropyl-4-phenyl-1H-pyrrol-2-yl]-ethyl)-2,2-dimethyl-[1,3]dioxan-4-yl)-acetic acid methyl ester (1 g, 99%), MS m/z 506 (M−Me+1), 400 MHz ¹H NMR (CDCl₃) δ 6.81-7.2 (m, 11H), 4.64 (m, 1H), 4.16 (m, 1H), 3.85 (s, 1H), 3.67 (br, 1H), 3.64 (s, 3H), 3.01 (br, 1H), 2.8 (br, 1H), 2.62 (br, 1H), 2.38 (abq, 2H), 1.68 (br, 1H), 1.59 (d, 6H), 1.5 (m, 1H), 1.3 (d, 6H).

Step D (6-{2-[5-(Acetylamino-methyl)-3-(4-fluoro-phenyl)-1-isopropyl-4-phenyl-1H-pyrrol-2-yl]-ethyl}-2,2-dimethy-1-[1,3]dioxan-4-yl)-acetic Acid Methyl Ester

A solution of 6-{2-[5-Aminomethyl-3-(4-fluoro-phenyl)-1-isopropyl-4-phenyl-1H-pyrrol-2-yl]-ethyl}-2,2-dimethyl-[1,3]dioxan-4-yl)-acetic acid methyl ester (0.2g) and acetic anhydride (0.1 g) in THF (5 mL) was stirred at RT for 30 min. The resulting crude (6-{2-[5-(Acetylamino-methyl)-3-(4-fluoro-phenyl)-1-isopropyl-4-phenyl-1H-pyrrol-2-yl]-ethyl}-2,2-dimethy-1-[1,3]dioxan-4-yl)-acetic acid methyl ester was chromatographed on silica gel with AcOEt/hexanes as an eluent to afford pure (6-{2-[5-(Acetylamino-methyl)-3-(4-fluoro-phenyl)-1-isopropyl-4-phenyl-1H-pyrrol-2-yl]-ethyl}-2,2-dimethy-1-[1,3]dioxan-4-yl)-acetic acid methyl ester (0.16g, 70%), MS m/z 565 (M+1), 400 MHz ¹H NMR (CDCl₃) δ 6.82-7.2 (m, 9H), 5.4 (br, NH), 4.51 (m, 1H), 4.47 (d, 2H), 4.15 (m, 1H), 3.76 (br, 1H), 3.64 (s, 3H), 2.8 (br, 1H), 2.66 (br, 1H), 2.38 (abq, 2H), 1.88 (s, 3H), 1.66 (br, 2H), 1.53 (d, 6H), 1.3 (d, 6H).

### Step E 7-[5-(Acetylamino-methyl)-3-(4-fluoro-phenyl)-1-isopropyl-4-phenyl-1H-pyrrol-2-yl]-3,5-dihydroxy-heptanoic Acid Methyl Ester

A solution of (6-{2-[5-(Acetylamino-methyl)-3-(4-fluoro-phenyl)-1-isopropyl-4-phenyl-1H-pyrrol-2-yl]-ethyl}-2,2-dimethy-1-[1,3]dioxan-4-yl)-acetic acid methyl ester (0.16g) and 0.4 m]L of 1N HCl in MeOH (5 mL) was stirred at RT for 30 min. After removal of the solvent under vacuo, afforded a gummy 7-[5-(Acetylamino-methyl)-3-(4-fluoro-phenyl)-1-isopropyl-4-phenyl-1H-pyrrol-2-yl]-3,5-dihydroxy-heptanoic acid methyl ester (0.13g, 100%), MS m/z 525 (M+1), HPLC t_{R}=14.12 min (92% pure) (90:10 to 10:90, 0.1% TFA water: 0.1% TFA acetonitrile, linear gradient over 20 min at 1.6 mL/min (λ=254 nm).

### Step F. Preparation of 7-[5-(Acetylamino-methyl)-3-(4-fluoro-phenyl)-1-isopropyl-4-phenyl-1H-pyrrol-2-yl]-3,5-dihydroxy-heptanoic Acid

7-[5-(Acetylamino-methyl)-3-(4-fluoro-phenyl)-1-isopropyl-4-phenyl-1H-pyrrol-2-yl]-3,5-dihydroxy-heptanoic acid methyl ester (0.12g) was dissolved in EtOH (5 mL) and THF (2 mL), and to this was added NaOH (1N, 0.22 mL). The reaction mixture was stirred at RT for 16 h, and the solvent was removed. The gummy residue was mixed with 10 mL of ether, stirred at RT for 16 h, and the solvent was removed. The gummy residue was mixed with 10 mL of ether, stirred at RT for 16h, and the precipitate was filtered to give an off-white solid, 7-[5-(Acetylamino-methyl)-3-(4-fluoro-phenyl)-1-isopropyl-4-phenyl-1H-pyrrol-2-yl]-3,5-dihydroxy-heptanoic acid (0.12 g, 99%), MS m/z 511 (M+1), HPLC t_{R}=12.84 min (96% pure) (90:10 to 10:90, 0.1% TFA water: 0.1% TFA acetonitrile, linear gradient over 20 min at 1.6 mL/min (λ=254 nm).

Following a similar method as described in Example 26, the following final products were made. Shown in Table IV.

TABLE IV
Final Products.

| IV-1 | 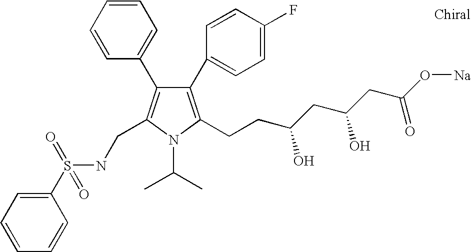 | 7-[5- (Benzenesulfonylamino- methyl)-3-(4-fluoro- phenyl)-1-isopropyl-4- phenyl-1H-pyrrol-2- yl]-3,5-dihydroxy- heptanoic acid, sodium salt | MS 607 | HPLC t_{R} =13.29 min (90% pure) |
|---|---|---|---|---|
| IV-2 | 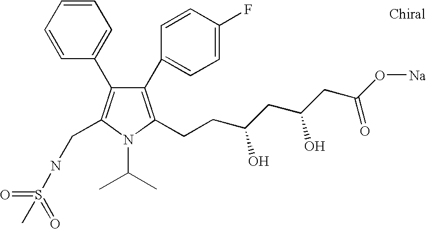 | 7-[3-(4-Fluoro-phenyl)- 1-isopropyl-5- (methanesulfonylamino- methyl)-4-phenyl-1H- pyrrol-2-yl]-3,5- dihydroxy-heptanoic acid, sodium salt | 547 | HPLC t_{R} = 10.96 min (90% pure) |
| IV-3 | 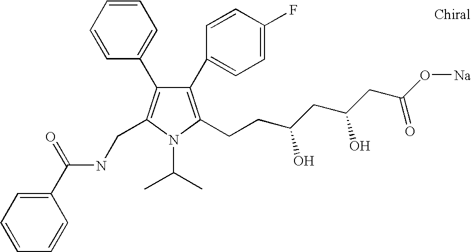 | 7-[5-(Benzoylamino- methyl)-3-(4-fluoro- phenyl)-1-isopropyl-4- phenyl-1H-pyrrol-2- yl]-3,5-dihydroxy- heptanoic acid, sodium salt | 573 | HPLC t_{R} = 15.4 min (88% pure) |

Note to Table IV: MS - m/z (M = 1)

### Example 27

### 4-(4-fluoro-phenyl)-5-formyl-1-isopropyl-3-phenyl-1H-pyrrole-2-carboxylic Acid

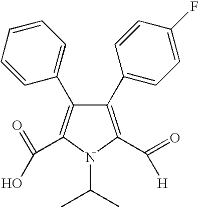 Step A

### 2-(4-Fluoro-phenyl)-1-phenyl-ethanone

To a solution of benzene (182 mL) at 0° C. was added AlCl₃ (46.4 g, 348 mmol). A portion of 4-fluorophenyl acetyl chloride (50.0 g, 290 mmol) was then added drop-wise over 30 min. Once the addition was complete, the reaction was allowed to warm to 25° C. and then heated to 50° C. for 8 hr. Subsequently, the reaction mixture was cooled to 25° C. and poured onto ice (400 g). To the resulting suspension in ice was added 1.0 N HCl (50 mL). The organic layer was separated and washed with 10% HCl, saturated NaHCO₃ and brine. The organic layer was then dried and concentrated to afford a solid that was washed twice with hexane (200 mL) and then dried under vacuum to afford 2-(4-fluoro-phenyl)-1-phenyl-ethanone (59.90 g, 97%):MS(APCI⁺): m/z 215.0 (M+H); H-NMR (CDCl₃) δ7.82 (d, 2H), 7.54-7.41 (m, 3H), 7.22-7.17 (m, 2H), 7.00-6.96 (m, 2H), 4.23 (s, 3H).

Step B

### 3-Dimethylamino-2-(4-fluoro-phenyl)-1-phenyl-propenone

To a solution of 2-(4-fluoro-phenyl)-1-phenyl-ethanone (56.90 g, 266 mmol) in toluene (400 mL) was added N,N-dimethlformamide dimethyl acetal (141 mL, 1.06 mol) and the reaction was heated to reflux for 16 hr. After cooling to 25° C., the solvent was removed under reduced pressure to afford an orange solid that was recrystallized from toluene (175 mL). The solid was isolated by filtration and washed with hexane (60 mL) to afford 3-dimethylamino-2-(4-fluoro-phenyl)-1-phenyl-propenone (57.1 g, 80%): H-NMR (CDCl₃) δ7.33-7.28 (m, 5H), 7.15 (s, 1H), 7.14-7.01 (m, 4H), 3.31 (s, 6H).

Step C

### Isopropylamino-acetic Acid Ethyl Ester

To a solution of ethyl bromoacetate (50.0 mL, 451 mmol) in toluene (400 mL) at 0° C. was added isopropylamine (115.2 mL, 1.35 mol). The reaction mixture was then heated to 95° C. for 5 hr and subsequently cooled to 25° C. The white precipitate which developed during the reaction was removed by filtration and the resulting filtrate was concentrated to a yellow oil which was subjected to vacuum distillation to provide isopropylamino-acetic acid ethyl ester (35.5 g, 54%) as a colorless liquid: H-NMR (CDCl₃) δ4.14 (q, 2H), 3.35 (s, 2H), 2.74 (sept, 1H), 1.22 (t, 3H), 1.01 (d, 6H).

Step D

### 4-(4-Fluoro-phenyl)-1-isopropyl-3-phenyl-1H-pyrrole-2-carboxylic Acid Ethyl Ester

To a mixture of isopropylamino-acetic acid ethyl ester and 3-dimethylamino-2-(4-fluoro-phenyl)-1-phenyl-propenone was added glacial AcOH (40 mL) and the reaction was heated to 125° C. for 2.5 hrs. The reaction mixture was then cooled to 25° C. and ether (100 mL) and water (100 mL) were added. The organic layer was separated and washed with saturated NaHCO₃ prior to drying over Na₂SO₄. The organic layer was then concentrated to afford a brown solid which was recrystallized from hexanes to afford 4.38 g of light brown needles; subsequently, the filtrate was concentrated and purified by silica gel chromatography (5% Et₂O/Hexane) to give an additional 0.90 g of product thus affording a combined (5.28 g, 81%) of 4-(4-fluoro-phenyl)-1-isopropyl-3-phenyl-1H-pyrrole-2-carboxylic acid ethyl ester: MS(APCI⁺): m/z 352.1 (M+H); H-NMR (CDCl₃) δ7.24-7.10(m, 6H), 7.01-6.97 (m, 2H), 6.81 (t, 2H), 5.40 (sept, 1H), 3.97 (q, 2H), 1.50 (d, 6H), 0.86 (t, 3H).

Steps E and F

### 4-(4-fluoro-phenyl)-5-formyl-1-isopropyl-3-phenyl-1H-pyrrole-2-carboxylic Acid

Using the method described previously (Example 1, Steps F and G) 4-(4-fluoro-phenyl)-1-isopropyl-3-phenyl-1H-pyrrole-2-carboxylic acid ethyl ester was converted to 4-(4-fluoro-phenyl)-5-formyl-1-isopropyl-3-phenyl-1H-pyrrole-2-carboxylic acid.

### Example 28

### (3R,5R)-7-[3-(4-fluoro-phenyl)-1-isopropyl-5-(4-methoxy-benzylcarbamoyl)-4-phenyl-1H-pyrrol-2-yl]-3,5-dihydroxy-heptanoic Acid Sodium Salt

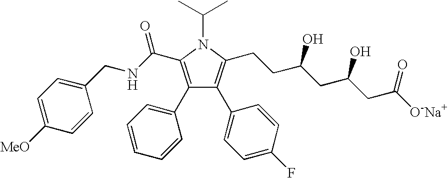 Step A

### 4-(4-Fluoro-phenyl)-5-formyl-1-isopropyl-3-phenyl-1H-pyrrole-2-carboxylic Acid 4-methoxy-benzylamide

To 4-(4-fluoro-phenyl)-5-formyl-1-isopropyl-3-phenyl-1H-pyrrole-2-carboxylic acid [from Example 1] (15.0 g, 42.7 mmol) was added thionyl chloride (100 mL) and the reaction mixture was heated to 75° C. for 2 hr after which time it was cooled to 25° C. and excess thionyl chloride was removed under reduced pressure. Subsequently, dichloromethane (250 mL) was added to the crude acid chloride and the solution was cooled to 0° C. 4-Methoxybenzyl amine (6.44 g, 47.0 mmol) and triethylamine (8.93 mL, 64.0 mmol) were then added and the reaction mixture was stirred at 0° C. for an additional 2 hrs. Saturated NaHCO₃ was added and organic layer separated, dried (Na₂SO₄) and concentrated. The product was purified by silica gel chromatography (10-20% EtOAc/hexane) to afford 4-(4-fluoro-phenyl)-5-formyl-1-isopropyl-3-phenyl-1H-pyrrole-2-carboxylic acid 4-methoxy-benzylamide (11.14 g, 55%): MS(APCI⁺): m/z 471.3 (M+H); H-NMR (CDCl₃) δ9.44 (s, 1H), 7.17-7.14 (m, 3H), 7.06-6.91 (m, 6H), 6.71 (d, 2H), 6.64 (d, 2-H), 5.58 (bs, 1H), 5.42 (m, 1H), 4.24 (d, 2H), 3.72 (s, 3H), 1.61 (d, 6H).

Step B

### 4-(4-Fluoro-phenyl)-5-hydroxymethyl-1-isopropyl-3-phenyl-1H-pyrrole-2-carboxylic Acid 4-methoxy-benzylamide

To a solution of 4-(4-fluoro-phenyl)-5-formyl-1-isopropyl-3-phenyl-1H-pyrrole-2-carboxylic acid 4-methoxy-benzylamide (11.1 g, 23.7 mmol) in THF (250 mL) at 0° C. was added 1.0 M lithium tri-tert-butoxyaluminohydride (28.4 mL, 28.4 mmol). The reaction was stirred for 30 min at 0° C. at which point TLC analysis indicated the reaction was complete and the solvent was removed under reduced pressure. To the reaction residue was added ethyl acetate (500 mL) and saturated NaHCO₃ (150 mL), and the organic layer was separated, dried (Na₂SO₄) and concentrated. The resulting oil was purified by silica gel chromatography (35% EtOAc/Hexane) to afford 4-(4-fluoro-phenyl)-5-hydroxymethyl-1-isopropyl-3-phenyl-1H-pyrrole-2-carboxylic acid 4-methoxy-benzylamide (4.64 g, 41%): H-NMR (CDCl₃) δ7.13-7.11 (m, 3H), 7.01-6.97 (m, 4H), 6.85-6.83 (m, 2H), 6.74-6.71 (m, 2H), 6.66-6.64 (m, 2H), 5.43 (bs, 1H), 4.99-4.96 (m, 1H), 4.58-4.57 (d, 2H), 4.20-4.18 (d, 2H), 3.72 (s, 3H), 1.67 (d, 6H).

Step C

### [3-(4-Fluoro-phenyl)-1-isopropyl-5-(4-methoxy-benzylcarbamoyl)-4-phenyl-1H-pyrrol-2-ylmethyl]-triphenyl-phosphonium Bromide

To a solution of 4-(4-fluoro-phenyl)-5-hydroxymethyl-1-isopropyl-3-phenyl-1H-pyrrole-2-carboxylic acid 4-methoxy-benzylamide (4.64 g, 9.82 mmol) in DCM (100 mL) was added triphenylphosphine hydrobromide (3.37 g, 9.82 mmol). The reaction was heated to 50° C. for 2.5 hr after which time all starting material was consumed as determined by TLC. The reaction solvent was removed under reduced pressure and the resulting yellow solid was dried under high vacuum for 12 hr to provide [3-(4-fluoro-phenyl)-1-isopropyl-5-(4-methoxy-benzylcarbamoyl)-4-phenyl-1H-pyrrol-2-ylmethyl]-triphenyl-phosphonium; bromide (7.82 g, 100%) in sufficient purity for use in the next step.

Step D

### (6-Formyl-2,2-dimethyl-[1,3]dioxan-4-yl)-acetic Acid Tert-Butyl Ester

To a solution of (6-hydroxymethyl-2,2-dimethyl-[1,3]dioxan-4-yl)-acetic acid tert-butyl ester (30.0 g, 115 mmol) at 0° C. in DCM:MeCN (10:1, 225 mL) was added 4 Å molecular sieves (55 g), 4-methylmorpholine N-oxide (20.3 g, 172.9 mmol) and tetrapropylammonium perruthenate (0.41 g, 1.15 mmol). The reaction was warmed from 0° C. to 25° C. over 0.5 hr and then stirred at that temperature for 5 hrs. Once complete, as determined by TLC, the reaction mixture was filtered through celite and the filtrate was concentrated to a brown oil that was purified by silica gel chromatography (20-70% EtOAc/Hexane) to provide (6-formyl-2,2-dimethyl-[1,3]dioxan-4-yl)-acetic acid tert-butyl ester (25.5 g, 86%): H-NMR (CDCl₃) □9.54 (s, 1H), 4.30-4.26 (m, 2H), 2.45-2.39 (m, 1H), 2.33-2.27 (m, 1H), 1.81-1.77 (m, 1H), 1.46-1.41 (m, 16H), 1.28-1.20 (m, 1H).

Step E

### (6-{2-[3-(4-Fluoro-phenyl)-1-isopropyl-5-(4-methoxy-benzylcarbamoyl)-4-phenyl-1H-pyrrol-2-yl]-vinyl}-2,2-dimethyl-[1,3]dioxan-4-yl)-acetic Acid Tert-Butyl Ester

To a solution of [3-(4-fluoro-phenyl)-1-isopropyl-5-(4-methoxy-benzylcarbamoyl)-4-phenyl-1H-pyrrol-2-ylmethyl]-triphenyl-phosphonium bromide (7.82g, 9.80 mmol) in THF (200 mL) at −78° C. was added 1.0 M NaHMDS (13.7 mL, 13.7 mmol). An orange color was noted as the base was added. The reaction mixture was stirred at −78° C. for 5 min after which time a solution of (6-formyl-2,2-dimethyl-[1,3]dioxan-4-yl)-acetic acid tert-butyl ester (2.79 g, 10.8 mmol) in THF (10 mL) was slowly added. After the addition, the reaction mixture was stirred at −78° C. for 30 min then allowed to warm to 25° C. over 1.5 hr. The reaction was quenched by drop-wise addition of saturated NH₄Cl. Ethyl acetate (250 mL) was then added and organic layer was separated, washed with water, dried (Na₂SO₄), concentrated. The crude product was purified by silica gel chromatography (15-20% EtOAc/Hexane) to afford (6-{2-[3-(4-Fluoro-phenyl)-1-isopropyl-5-(4-methoxy-benzylcarbamoyl)-4-phenyl-1H-pyrrol-2-yl]-vinyl}-2,2-dimethyl-[1,3]dioxan-4-yl)-acetic acid tert-butyl ester (5.11 g, 75%) as a mixture of cis/trans olefin isomers.

Step F

### (3R,5R)-7-[3-(4-Fluoro-phenyl)-1-isopropyl-5-(4-methoxy-benzylcarbamoyl)-4-phenyl-1H-pyrrol-2-yl]-3,5-dihydroxy-heptanoic Acid Tert-Butyl Ester

To a solution of (6-{2-[3-(4-fluoro-phenyl)-1-isopropyl-5-(4-methoxy-benzylcarbamoyl)-4-phenyl-1H-pyrrol-2-yl]-vinyl}-2,2-dimethyl-[1,3]dioxan-4-yl)-acetic acid tert-butyl-ester (5.11 g, 7.33 mmol) in MeOH (200 mL) was added 10% Pd—C (500 mg). The reaction vessel was evacuated and filled with hydrogen gas (50 psi) for 3 hours. The reaction mixture was then filtered through a pad of celite and to the filtrate was added 1N HCl (10 mL) and the solution was stirred for 3 hrs at 25° C. Subsequently, the reaction solvent was removed under reduced pressure and ethyl acetate (200 mL) and saturated NaHCO₃ (100 mL) were added. The organic layer was separated, washed with brine, dried (Na₂SO₄) and concentrated. The crude product was purified by silica gel chromatography (30-70% EtOAc/Hexane) to provide (3R,5R)-7-[3-(4-fluoro-phenyl)-1-isopropyl-5-(4-methoxy-benzylcarbamoyl)-4-phenyl-1H-pyrrol-2-yl]-3,5-dihydroxy-heptanoic acid tert-butyl ester (3.74 g, 77%): H-NMR (CDCl₃) δ7.10-7.09 (m, 3H), 6.98-6.91 (m, 4H), 6.86-6.82 (m, 2H), 6.73-6.72 (m, 2H), 6.65-6.63 (m, 2H), 5.39-5.42 (m, 1H), 4.72-4.79 (m, 1H), 4.18-4.16 (d, 2H), 4.03-4.07 (m, 1H), 3.71 (s, 3H), 2.81-2.69 (m, 2H), 2.27-2.26 (m, 2H), 1.65-1.62 (m, 6H), 1.61-1.22 (4H), 1.41 (s, 9H).

Step G

### (3R,5R)-7-[3-(4-fluoro-phenyl)-1-isopropyl-5-(4-methoxy-benzylcarbamoyl)-4-phenyl-1H-pyrrol-2-yl]-3,5-dihydroxy-heptanoic Acid Sodium Salt

To a solution of 7-[3-(4-fluoro-phenyl)-1-isopropyl-5-(4-methoxy-benzylcarbamoyl)-4-phenyl-1H-pyrrol-2-yl]-3,5-dihydroxy-heptanoic acid tert-butyl ester (3.39 g, 5.15 mmol) in MeOH (100 mL) was added 1.03 N NaOH (5.11 mL, 5.25 mmol) and the reaction was stirred at 25° C. for 48 hr after which time the reaction was solvent was removed under reduced pressure. The resulting solid was then azeotroped with toluene (3×100 mL) and triturated with diethyl ether to provide a light yellow solid that was dried under vacuum at 60° C. to afford (3R,5R)-7-[3-(4-fluoro-phenyl)-1-isopropyl-5-(4-methoxy-benzylcarbamoyl)-4-phenyl-1H-pyrrol-2-yl]-3,5-dihydroxy-heptanoic acid sodium salt (2.99 g, 93%): MS(APCI⁺): m/z 603.6 (M+H); H-NMR (DMSO-d₆) δ 8.27 (t, 1H), 7.40 (s, 1H), 7.06-6.89 (m, 8H), 6.82-6.80 (d, 2H), 6.67-6.65 (d, 2H), 4.74 (bs, 1H), 4.49-4.46 (m, 1H), 4.07-4.06 (d, 2H), 3.68-3.64 (m, 1H), 3.64 (s, 3H), 2.65-2.63 (m, 1H), 2.42-2.38 (m, 1H), 1.98-1.94 (m, 1H), 1.78-1.72 (m, 1H), 1.58-1.18 (m, 4H), 1.43 (d, 6H).

Examples 29-53 were prepared following a similar procedure as described in Example 28. Shown are various replacements for the 4-methoxy-benzyl substituent, or, where NR⁶R⁷ forms a ring, replacements for methoxy-benzyl carbamoyl. Specific experimental details for Examples 30, 40 and 44 follow thereafter.

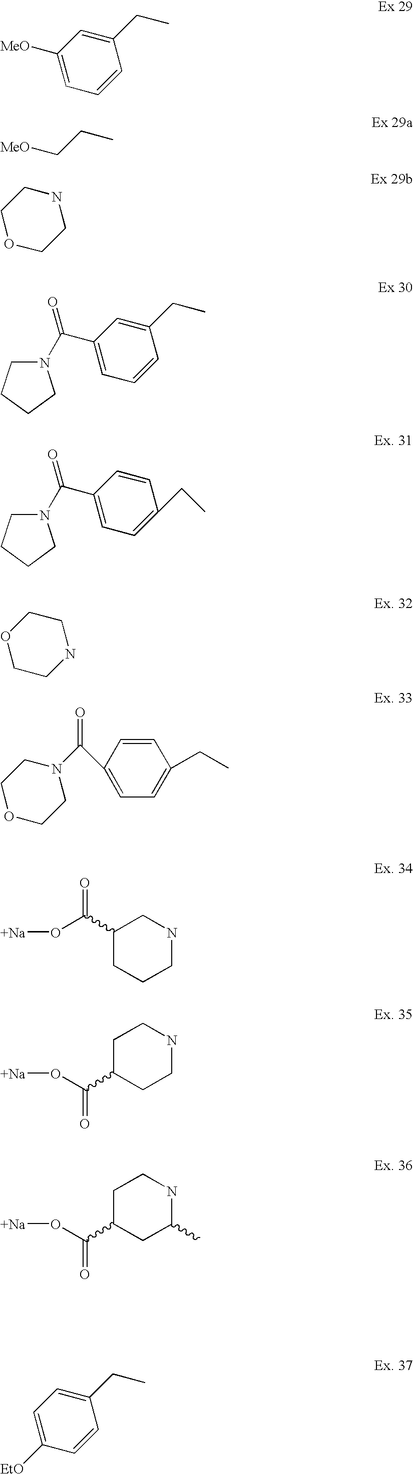

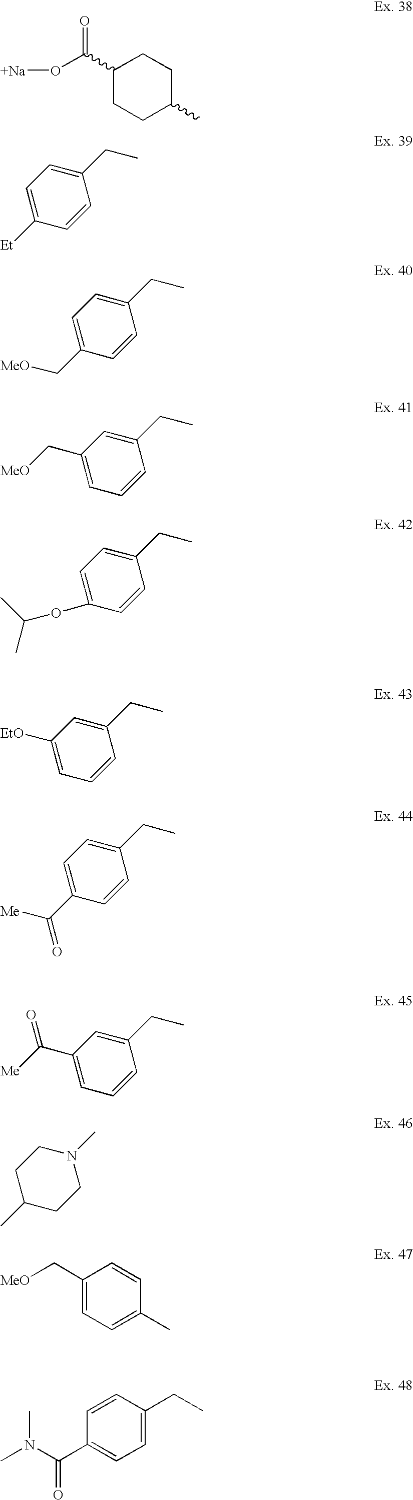

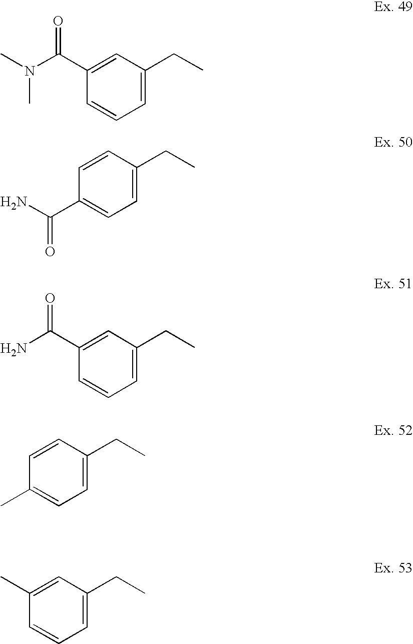

### Example 30

### (3R,5R)-7-{3-(4-fluoro-phenyl)-1-isopropyl-4-phenyl-5-[3-(pyrrolidine-1-carbonyl)-benzylcarbamoyl]-1H-pyrrol-2-yl}-3,5-dihydroxy-heptanoic Acid Sodium Salt

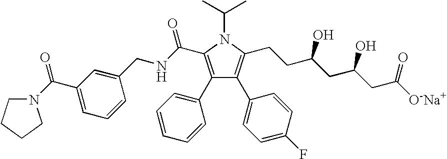 Step A

### (3-Chloromethyl-phenyl)-pyrrolidin-1-yl-methanone

To a solution of 3-(chloromethyl) benzoyl chloride (3.00 g, 15.9 mmol) in DCM (100 mL) at 0° C. was added pyrrolidine (1.39 mL, 16.7 mmol) followed by triethylamine (2.65 mL, 19.0 mmol). The reaction was stirred at 0° C. for 30 min and then allowed to warm to 25° C. and stirred for an additional 2 hrs. The reaction was quenched by addition of saturated NaHCO₃ and organic layer was separated, dried (Na₂SO₄) and concentrated. Crude product was purified by silica gel chromatography (100% EtOAc) to provide (3-chloromethyl-phenyl)-pyrrolidin-1-yl-methanone (2.28 g, 64%): H-NMR (CDCl₃) δ7.52 (s, 1H), 7.48-7.33 (m, 3H), 4.56 (s, 2H), 3.63-3.59 (m, 2H), 3.41-3.37 (m, 2H), 1.96-1.83 (m, 4H).

Step B

### (3-Aminomethyl-phenyl)-pyrrolidin-1-yl-methanone Hydrochloride Salt

A solution of (3-chloromethyl-phenyl)-pyrrolidin-1-yl-methanone (2.28-g, 10.2 mmol) in EtOH (100 mL) was cooled to 0° C. and a stream of ammonia gas was bubbled through the reaction mixture for 15 min. The reaction vessel was then sealed and allowed to warm to 25° C. and stirred at that temperature for 48 hrs. Subsequently, the reaction solvent was removed under reduced pressure to provide (3-aminomethyl-phenyl)-pyrrolidin-1-yl-methanone hydrochloride salt (2.39 g, 97%) as a white solid of sufficient purity for use without further purification: MS(APCI⁺): m/z 224.6 (M+H);

Step C

### 7-{3-(4-fluoro-phenyl)-1-isopropyl-4-phenyl-5-[3-(pyrrolidine-1-carbonyl)-benzylcarbamoyl]-1H-pyrrol-2-yl}-3,5-dihydroxy-heptanoic Acid Sodium Salt

Using the method of Example 28 (Steps A-G), (3-aminomethyl-phenyl)-pyrrolidin-1-yl-methanone hydrochloride salt was converted to (3R,5R)-7-{3-(4-fluoro-phenyl)-1-isopropyl-4-phenyl-5-[3-(pyrrolidine-1-carbonyl)-benzylcarbamoyl]-1H-pyrrol-2-yl)-3,5-dihydroxy-heptanoic acid sodium salt: MS(APCI⁺): m/z 670.2 (M+H); H-NMR (DMSO-d₆) □8.41 (bs, 1H), 7.59-7.49 (m, 3H), 7.24-6.89 (m, 10H), 4.50-4.46 (m, 1H), 4.17 (s, 2H), 3.68-3.66 (m, 1H), 3.54-3.52 (m, 1H), 3.29-3.11 (m, 7H), 2.64-2.62 (m, 1H), 1.99-1.94 (m, 1H), 1.93-1.82 (m, 4H), 1.52-1.17 (m, 10H).

### Example 40

### (3R,5R)-7-[3-(4-fluoro-phenyl)-1-isopropyl-5-(4-methoxymethyl-benzylcarbamoyl)-4-phenyl-1H-pyrrol-2-yl]-3,5-dihydroxy-heptanoic Acid Sodium Salt

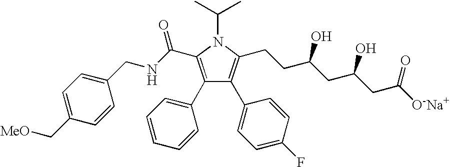 Step A

### 4-Methoxymethyl-benzonitrile

To a solution of 4-bromomethyl-benzonitrile (10.0 g, 51.0 mmol) in THF (50 mL) at 0° C. was slowly added NaOMe (14.0 mL of 25% solution in MeOH, 61.2 mmol). Precipitation was noted after addition of NaOMe. The reaction was warmed to 25° C. and stirred for 1 hour. Saturated NH₄Cl was added and the reaction mixture was extracted with DCM. The organic extracts were dried over MgSO₄ and concentrated to a solid which was dried under vacuum for 18 hr to give 4-methoxymethyl-benzonitrile (6.35 g, 85%) which did not require further purification: MS(APCI⁺): m/z 147.9 (M+H); H-NMR (CDCl₃) δ 7.60 (d, 2H), 7.41 (d, 2H), 4.47 (s, 2H), 3.38 (s, 3H).

Step B

### 4-Methoxymethyl-benzyl Amine

To a solution of 4-methoxymethyl-benzonitrile (6.35 g, 43.1 mmol) in MeOH/NH₃ (100 mL) was added Raney-nickel (500 mg). The reaction vessel was evacuated and pressurized with hydrogen gas (50 psi) for 16 hrs. The reaction was then filtered through a pad of celite and the filtrate was concentrated to provide 4-methoxymethyl-benzylamine (6.20 g, 41.0 mmol) which did not require further purification: MS(APCI⁺): m/z 151.9 (M+H); H-NMR (CDCl₃) δ 7.25-7.16 (m, 4H), 4.31 (s, 2H), 3.65-3.59 (m, 2H), 3.20 (s, 3H), 1.65 (bs, 2H).

Step C

### (3R,5R)-7-[3-(4-fluoro-phenyl)-1-isopropyl-5-(4-methoxymethyl-benzylcarbamoyl)-4-phenyl-1H-pyrrol-2-yl]-3,5-dihydroxy-heptanoic Acid Sodium Salt

4-Methoxymethyl-benzylamine (from Step B) was converted to (3R,5R)-7-[3-(4-fluoro-phenyl)-1-isopropyl-5-(4-methoxymethyl-benzylcarbamoyl)-4-phenyl-1H-pyrrol-2-yl]-3,5-dihydroxy-heptanoic acid sodium salt using the method described for Example 28 MS(APCI⁺): m/z 617.3 (M+H); H-NMR (DMSO-d₆) δ 8.35 (bs, 1H), 7.06-6.92 (m, 11H), 6.83 (d, 2H), 4.48-4.45 (m, 1H), 4.28 (s, 2H), 4.14 (s, 2H), 3.70-3.66 (m, 1H), 3.54-3.52 (m, 1H), 3.36-3.11 (m, 3H), 3.19 (s, 3H), 2.63-2.59 (m, 1H), 2.45-2.39 (m, 1H), 1.98-1.94 (m, 1H), 1.79-1.73 (m, 1H), 1.53-1.18 (m, 10H).

### Example 44

### (3R,5R)-7-[5-(4-acetyl-benzylcarbamoyl)-3-(4-fluoro-phenyl)-1-isopropyl-4-phenyl-1H-pyrrol-2-yl]-3,5-dihydroxy-heptanoic Acid Sodium Salt

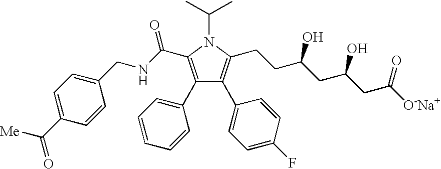 Step A

### 4-(2-Methyl-[1,3]dioxolan-2-yl)-benzonitrile

To a solution of 4-acetyl-benzonitrile (10.0 g, 68.9 mmol) in benzene (125 mL) was added ethylene glycol (5.76 mL, 103 mmol) and BF₃.Et₂O (1.0 g, 7.05 mmol). The reaction was heated to reflux with a Dean-Stark apparatus in place for 16 hrs. After cooling to 25° C., the reaction mixture was transferred to a separatory funnel and washed with saturated NaHCO₃. The organic layer was then dried (Na₂SO₄) and concentrated to an oil which was purified by silica gel chromatography (10-20% EtOAc/Hex) to give 4-(2-methyl-[1,3]dioxolan-2-yl)-benzonitrile (10.7 g, 82%): H-NMR (CDCl₃) δ 7.61-7.54 (m, 4H), 4.06-3.97 (m, 2H), 3.75-3.66 (m, 2H), 1.58 (s, 3H).

Step B

### 4-(2-Methyl-[1,3]dioxolan-2-yl)-benzyl Amine

To a solution of 4-(2-methyl-[1,3]dioxolan-2-yl)-benzonitrile (5.00 g, 26.4 mmol) in MeOH/NH₃ (100 mL) was added Raney-nickel (500 mg). The reaction vessel was pressurized with hydrogen gas for 5 hrs after which time the reaction mixture was filtered through celite, and the filtrate was concentrated to afford 4-(2-Methyl-[1,3]dioxolan-2-yl)-benzylamine (4.91, 96%) in sufficient purity for use in the next reaction: H-NMR (CDCl₃) δ 7.40 (d, 2H), 7.23 (d, 2H), 4.00-3.94 (m, 2H), 3.77-3.71 (m, 4H), 3.40 (s, 3H), 1.60 (bs, 2H).

Step C

### (3R,5R)-7-[5-(4-acetyl-benzylcarbamoyl)-3-(4-fluoro-phenyl)-1-isopropyl-4-phenyl-1H-pyrrol-2-yl]-3,5-dihydroxy-heptanoic Acid Sodium Salt

4-(2-Methyl-[1,3]dioxolan-2-yl)-benzylamine (from Step B) was converted to 7-[5-(3-acetyl-benzylcarbamoyl)-3-(4-fluoro-phenyl)-1-isopropyl-4-phenyl-1H-pyrrol-2-yl]-3,5-dihydroxy-heptanoic acid sodium salt according to the method described for Example 28 MS(APCI⁺): m/z 615.3 (M+H); H-NMR (DMSO-d₆) δ 8.44 (t, 1H), 7.68-7.65 (m, 3H), 7.09-6.92 (m, 10H), 4.71-4.79 (m, 1H), 4.51-4.47 (m, 1H), 4.20 (d, 2H), 3.62-3.68 (m, 1H), 3.57-3.51 (m, 1H), 2.69-2.59 (m, 1H), 2.48 (s, 3H), 2.47-2.41 (m, 1H), 1.94-1.90 (m, 1H), 1.75-1.69 (m, 1H), 1.45-1.18 (m, 10H).

### Example 54

### (3R,5R)-7-[3-(4-Fluoro-phenyl)-5-(4-hydroxymethyl-benzylcarbamoyl)-1-isopropyl-4-phenyl-1H-pyrrol-2-yl]-3,5-dihydroxy-heptanoic Acid Sodium Salt

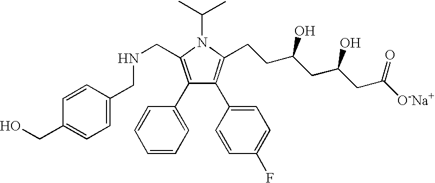 Step A

### (4-Aminomethyl-phenyl)-methanol Hydrochloride Salt

4-Hydroxymethyl-benzonitrile (2.0 g, 15 mol) was reduced using Raney nickel (0.5 g) and hydrogen (50 psi) in MeOH:NH₃ (100 ml) for 20 hours. The reaction mixture was filtered through a pad of celite. The filtrate was concentrated under reduced pressure to afford (4-aminomethyl-phenyl)-methanol hydrochloride salt (2.01 g, 98%) as a white solid of sufficient purity for use without further purification: MS(APCI⁺): m/z 138.3 (M+H); H-NMR (DMSO-d₆) δ 7.20 (s, 4H), 5.04 (s, 2H), 4.42 (s, 2H), 3.83 (s, 1H), 2.45 (s, 2H).

Step B

### 4-(4-Fluoro-phenyl)-5-formyl-1-isopropyl-3-phenyl-1H-pyrrole-2-carboxylic Acid 4-hydroxymethyl-benzylamide

Thionyl chloride (10 ml) was added to 4-(4-fluoro-phenyl)-5-formyl-1-isopropyl-3-phenyl-1H-pyrrole-2-carboxylic acid (3.0 g, 8.54 mmol). A catalytic amount of DMF was then added to the reaction mixture over 1 minute. The reaction mixture was refluxed for 1.5 hr and was then cooled to 25° C. The organic solvent was concentrated under reduced pressure and cooled to −10° C. (4-aminomethyl-phenyl)-methanol (1.78 g) in EtOAc (10 ml) was then added to acid chloride, followed by (1:4) mixture of H₂O and EtOAc (50 ml), and solid sodium carbonate at −10° C. The reaction mixture was stirred for 2 hr at 0° C. and was allowed to warm up to 25° C. for 12 hr. The organic mixture was diluted with the mixture of EtOAc and H₂O (5:1, 120 ml) and the separated organic solvent was washed with 1N HCl, saturated NaHCO₃ and brine, dried over anhydrous magnesium sulfate and was concentrated under reduced pressure. The crude product was purified by silica gel chromatography (50% ethyl acetate in hexane) to afford desired 4-(4-fluoro-phenyl)-5-formyl-1-isopropyl-3-phenyl-1H-pyrrole-2-carboxylic acid 4-hydroxymethyl-benzylamide (3.91 g, 97%): MS(APCI⁺): m/z 471.1 (M+H); H-NMR (CDCl₃) δ 9.45 (s, 1H), 7.21-7.12 (m, 5H), 7.07-6.98 (m, 4H), 6.92 (t, 2H), 6.78 (d, 2H), 5.63 (t, 1H), 5.49-5.41 (m, 1H), 4.61 (s, 2H), 4.31 (s, 2H), 1.62-(d, 6H).

Step C

### 2,2-Dimethyl-propionic Acid 4-({[4-(4-fluoro-phenyl)-5-formyl-1-isopropyl-3-phenyl-1H-pyrrole-2-carbonyl]-amino}-methyl)-benzyl Ester

To a solution of 4-(4-fluoro-phenyl)-5-formyl-1-isopropyl-3-phenyl-1H-pyrrole-2-carboxylic acid 4-hydroxymethyl-benzylamide (3.91 g, 8.31 mmol) in DCM (100 ml) was added triethylamine (12 ml, 83.1 mmol) at 0° C. Pivoyl chloride (3.1 ml, 24.93 mmol) was added dropwise followed by 4-(dimethylamino)pyridine (51 mg, 0.42 mmol) at 0° C. The reaction mixture was stirred at 25° C. for 4 hrs. The reaction was quenched with water, diluted with ether, and the layers were separated. The organic layer was washed with 10% HCl, saturated NaHCO₃, brine, dried over anhydrous sodium sulfate, filtered, and concentrated under reduced pressure. The crude product was purified by silica gel chromatography (10-50% EtOAc/Hexane) to afford desired 2,2-dimethyl-propionic acid 4-({[4-(4-fluoro-phenyl)-5-formyl-1-isopropyl-3-phenyl-1H-pyrrole-2-carbonyl]-amino}-methyl)-benzyl ester (4.20g, 92%):MS(APCI⁺): m/z 555.1 (M+H); H-NMR (DMSO-D₆) δ 9.45 (s, 1H), 7.19-7.09 (m, 5H), 7.07-7.02 (m, 2H), 7.00-6.98 (m, 2H), 6.94-6.88 (m, 2H), 6.79 (d, 2H), 5.64 (t, 1H), 5.50-5.40 (m, 1H), 5.00 (s, 2H), 4.31 (d, 2H), 1.62 (d, 6H), 1.18 (s, 9H).

Step D

### 2,2-Dimethyl-propionic Acid 4-({[4-(4-fluoro-phenyl)-5-hydroxymethyl-1-isopropyl-3-phenyl-1H-pyrrole-2-carbonyl]-amino)-methyl)-benzyl Ester

To a solution of 2,2-dimethyl-propionic acid 4-({[4-(4-fluoro-phenyl)-5-formyl-1-isopropyl-3-phenyl-1H-pyrrole-2-carbonyl]-amino}-methyl)-benzyl ester (4.0 g, 7.2 mmol) in THF:MeOH (1:1, 200 ml) at −101C was added sodium borohydride (300 mg, 7.9 mmol). The reaction was stirred at that temperature for 1 hr. The organic solvent was then partially removed and dichlomethane (200 ml) was added. The organic layer was separated and washed with aqueous saturated sodium bicarbonate and brine, dried over anhydrous sodium sulfate, filtered and concentrated under reduced pressure. The crude product was purified by silica chromatography (20-70% ethyl acetate in hexane) to afford desired 2,2-dimethyl-propionic acid 4-({[4-(4-fluoro-phenyl)-5-hydroxymethyl-1-isopropyl-3-phenyl-1H-pyrrole-2-carbonyl]-amino}-methyl)-benzyl ester (3.77 g, 94%): MS(APCI⁺): m/z 557.3 (M+H); H-NMR (CDCl₃) δ 7.14-7.07 (m, 5H), 7.01-6.95 (m, 4H), 6.89-6.79 (m, 4H), 5.55-5.47 (m, 1H), 5.03-4.96 (m, 3H), 4.58 (d, 2H), 4.26 (d, 2H), 1.67 (d, 6H), 1.17 (s, 9H).

Step E

### [5-[4-(2,2-Dimethyl-propionyloxymethyl)-benzylcarbamoyl]-3-(4-fluoro-phenyl)-1-isopropyl-4-phenyl-1H-pyrrol-2-ylmethyl]-triphenyl-phosphonium Bromide

To a solution of 2,2-dimethyl-propionic acid 4-({[4-(4-fluoro-phenyl)-5-hydroxymethyl-1-isopropyl-3-phenyl-1H-pyrrole-2-carbonyl]-amino)-methyl)-benzyl ester (3.77 g, 6.77 mmol) in dichloromethane (200 ml) was added triphenylphosphine hydrobromide (2.32 g, 6.77 mmol). The reaction mixture was heated to 50° C. for 1.5 hrs after which time no starting material was detected by TLC analysis. The reaction solvent was removed under reduced pressure and dried with azeotropic evaporation three times and under high vacuum for 12 hrs to provide desired [5-[4-(2,2-dimethyl-propionyloxymethyl)-benzylcarbamoyl]-3-(4-fluoro-phenyl)-1-isopropyl-4-phenyl-1H-pyrrol-2-ylmethyl]-triphenyl-phosphonium bromide (5.97 g, 100%) in sufficient purity for use in the next step.

Step F

### 2,2-Dimethyl-propionic Acid 4-({[5-[2-(6-tert-butoxycarbonylmethyl-2,2-dimethyl-[1,3]dioxan-4-yl)-vinyl]-4-(4-fluoro-phenyl)-1-isopropyl-3-phenyl-1H-pyrrole-2-carbonyl]-amino)-methyl)-benzyl Ester

To a solution of [5-[4-(2,2-dimethyl-propionyloxymethyl)-benzylcarbamoyl]-3-(4-fluoro-phenyl)-1-isopropyl-4-phenyl-1H-pyrrol-2-ylmethyl]-triphenyl-phosphonium bromide (5.97 g, 6.77 mmol) in THF (100 ml) and DMSO (5 ml) at −78° C. was added dropwise NaHMDS (1.0 M in THF, 7.45 ml). Reaction was stirred at −78° C. for 5 min after which time a solution of (6-formyl-2,2-dimethyl-[1,3]dioxan-4-yl)-acetic acid tert-butyl ester (From Example 28, step D; 1.92 g, 7.45 mmol) in THF (15 ml), was added dropwise. The reaction mixture was stirred at −78° C. for 30 min then allowed to warm to 25° C. over 1.5 hr. The reaction was quenched by dropwise addition of aqueous saturated ammonium chloride. EtOAc (200 ml) was then added and the separated organic layer was washed with water, dried over anhydrous sodium sulfate, concentrated under reduced pressure. The crude oil was purified by silica gel chromatography (20-80% ethyl acetate:hexane) to afford 2,2-dimethyl-propionic acid 4-({[5-[2-(6-tert-butoxycarbonylmethyl-2,2-dimethyl-[1,3]dioxan-4-yl)-vinyl]-4-(4-fluoro-phenyl)-1-isopropyl-3-phenyl-1H-pyrrole-2-carbonyl]-amino)-methyl)-benzyl ester (4.95g, 94%) as a mixture of cis/trans olefin isomers:MS(APCI⁺): m/z 781.3 (M+H).

Step G

### (6-{2-[3-(4-Fluoro-phenyl)-5-(4-hydroxymethyl-benzylcarbamoyl)-1-isopropyl-4-phenyl-1H-pyrrol-2-yl]-vinyl)-2,2-dimethyl-[1,3]dioxan-4-yl)-acetic Acid Tert-Butyl Ester

To a 2,2-dimethyl-propionic acid 4-({[5-[2-(6-tert-butoxycarbonylmethyl-2,2-dimethyl-[1,3] dioxan-4-yl)-vinyl]-4-(4-fluoro-phenyl)-1-isopropyl-3-phenyl-1H-pyrrole-2-carbonyl]-amino)-methyl)-benzyl ester (3.11 g, 3.98 mmol) in MeOH (50 ml), aqueous NaOH (1.03 N, 6.20 ml, 6.37 mmol) was added with stirring at room temperature for 24 hrs. The reaction mixture was poured into DCM and saturated NaHCO₃, separated, dried over anhydrous sodium sulfate, concentrated under reduced pressure. The crude product was purified by silica gel chromatography (20-50% ethyl acetate/hexane) to afford desired (6-{2-[3-(4-fluoro-phenyl)-5-(4-hydroxymethyl-benzylcarbamoyl)-1-isopropyl-4-phenyl-1H-pyrrol-2-yl]-vinyl}-2,2-dimethyl-[1,3]dioxan-4-yl)-acetic acid tert-butyl ester (2.01 g, 72%) as a mixture of cis/trans olefin isomers: MS(APCI⁺): m/z 697.2 (M+H).

Step H

### (6-{2-[3-(4-Fluoro-phenyl)-5-(4-hydroxymethyl-benzylcarbamoyl)-1-isopropyl-4-phenyl-1H-pyrrol-2-yl]-ethyl)-2,2-dimethyl-[1,3]dioxan-4-yl)-acetic Acid Tert-Butyl Ester

(6-{2-[3-(4-Fluoro-phenyl)-5-(4-hydroxymethyl-benzylcarbamoyl)-1-isopropyl-4-phenyl-1H-pyrrol-2-yl]-vinyl}-2,2-dimethyl-[1,3]dioxan-4-yl)-acetic acid tert-butyl ester (1.20 g, 1.72 mmol) was dissolved in ethanol (16 ml) and reduced with 10% palladium on carbon (0.25 g) in the presence of n-butyl amine (700 mg, 5%) under hydrogen for 6 hr. The reaction mixture was filtered through a pad of celite and concentrated under reduced pressure. The crude product was purified by silica gel chromatography (35-55% EtOAc/Hexane) to afford (6-{2-[3-(4-fluoro-phenyl)-5-(4-hydroxymethyl-benzylcarbamoyl)-1-isopropyl-4-phenyl-1H-pyrrol-2-yl]-ethyl}-2,2-dimethyl-[1,3]dioxan-4-yl)-acetic acid tert-butyl ester (1.05 g, 82%): MS(APCI⁺): m/z 699.6 (M+H).

Step I

### 7-[3-(4-Fluoro-phenyl)-5-(4-hydroxymethyl-benzylcarbamoyl)-1-isopropyl-4-phenyl-1H-pyrrol-2-yl]-3,5-dihydroxy-heptanoic Acid Tert-Butyl Ester

To a solution of (6-{2-[3-(4-fluoro-phenyl)-5-(4-hydroxymethyl-benzylcarbamoyl)-1-isopropyl-4-phenyl-1H-pyrrol-2-yl]-ethyl}-2,2-dimethyl-[1,3]dioxan-4-yl)-acetic acid tert-butyl ester (100 mg, 0.14 mmol) in the mixture of MeOH and H₂O mixture (9:1, 6.0 ml), aqueous 1N HCl (143 ul, 0,14 mmol) was added. The reaction mixture was stirred at room temperature for 12 hr. The reaction mixture was diluted with DCM (50 ml) and saturated NaHCO₃ (20 ml) and organic phase was separated. The organic solvent was washed with brine, dried over anhydrous sodium sulfate, filtered, concentrated under reduced pressure. The crude product was purified by silica gel chromatography (50-70% EtOAc/Hexane) to afford desired 7-[3-(4-fluoro-phenyl)-5-(4-hydroxymethyl-benzylcarbamoyl)-1-isopropyl-4-phenyl-1H-pyrrol-2-yl]-3,5-dihydroxy-heptanoic acid tert-butyl ester (54 mg, 57%): MS(APCI⁺): m/z 659.2 (M+H).

Step J

### (3R,5R)-7-[3-(4-Fluoro-phenyl)-5-(4-hydroxymethyl-benzylcarbamoyl)-1-isopropyl-4-phenyl-1H-pyrrol-2-yl]-3,5-dihydroxy-heptanoic Acid Sodium Salt

To a solution of 7-[3-(4-fluoro-phenyl)-5-(4-hydroxymethyl-benzylcarbamoyl)-1-isopropyl-4-phenyl-1-H-pyrrol-2-yl]-3,5-dihydroxy-heptanoic acid tert-butyl ester (54 mg, 0.82 mmol) in methanol (5 ml), aqueous NaOH (1.03 N, 84 ul, 0.86 mmol) was added. The reaction mixture was stirred over 48 hr. After hydrolysis completed, the reaction mixture was concentrated under reduced pressure. The resulting solid was then azeotroped toluene (3×10 ml) and triturated with diethyl ether to provide a light yellow solid that was under vacuum at 60° C. to afford 7-[3-(4-fluoro-phenyl)-5-(4-hydroxymethyl-benzylcarbamoyl)-1-isopropyl-4-phenyl-1H-pyrrol-2-yl]-3,5-dihydroxy-heptanoic acid sodium salt (35 mg, 65%).

MS(APCI⁺): m/z 603.3 (M+H).

### Example 55

### (3R,5R)-7-[5-(4-Dimethylaminomethyl-benzylcarbamoyl)-3-(4-fluoro-phenyl)-1-isopropyl-4-phenyl-1H-pyrrol-2-yl)-3,5-dihydroxy-heptanoic Acid Sodium Salt

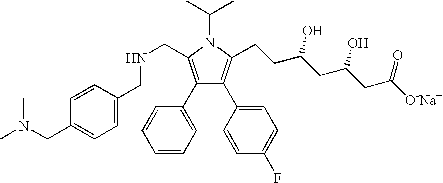 Step A

### (6-{2-[3-(4-Fluoro-phenyl)-1-isopropyl-5-(4-methanesulfonyloxymethyl-benzylcarbamoyl)-4-phenyl-1H-pyrrol-2-yl]-ethyl}-2,2-dimethyl-[1,3]dioxan-4-yl)-acetic Acid Tert-Butyl Ester

To a stirred solution of (6-{2-[3-(4-fluoro-phenyl)-5-(4-hydroxymethyl-1-benzylcarbamoyl)-1-isopropyl-4-phenyl-1H-pyrrol-2-yl]-ethyl}-2,2-dimethyl-[1,3]dioxan-4-yl)-acetic acid tert-butyl ester (300 mg, 0.43 mmol; from Example 54 and Et₃N (120 ul, 0.86 mmol) in 50 ml of DCM was added mesyl chloride (37 ul, 0.47 mmol) at 0° C. The solution was stirred for 1 hr at 25° C. during which time the starting material disappeared. The reaction mixture was washed with aqueous NaHCO₃ (saturated) and brine and dried over Na₂SO₄, filtered and concentrated. The crude product (0.33 mg, 99%) was used without further purification.

Step B

### 6-{2-[5-(4-Dimethylaminomethyl-benzylcarbamoyl)-3-(4-fluoro-phenyl)-1-isopropyl-4-phenyl-1H-pyrrol-2-yl]-ethyl}-2,2-dimethyl-[1,3]dioxan-4-yl)-acetic Acid Tert-Butyl Ester

To the solution of (6-{2-[3-(4-fluoro-phenyl)-1-isopropyl-5-(4-methanesulfonyloxymethyl-benzylcarbamoyl)-4-phenyl-1H-pyrrol-2-yl]-ethyl}-2,2-dimethyl-[1,3]dioxan-4-yl)-acetic acid tert-butyl ester (330 mg, 0.42 mmol) in DCM (50 ml), TEA (4.2 mmol, 7.2 ml) and dimethylamine (2.0 M in THF, 2.1 ml, 4.2 mmol) were added. The reaction mixture was stirred for over night at 25° C. and diluted with DCM and quenched by addition of aqueous NaHCO₃ (saturated). The separated organic layer was dried over Na₂SO₄, filtered, and concentrated. The product was purified by silica gel chromatography (0-10% MeOH/DCM) to afford 6-{2-[5-(4-dimethylaminomethyl-benzylcarbamoyl)-3-(4-fluoro-phenyl)-1-isopropyl-4-phenyl-1H-pyrrol-2-yl]-ethyl}-2,2-dimethyl-[1,3]dioxan-4-yl)-acetic acid tert-butyl ester (250 mg, 0.34 mmol, 81%):MS(APCI⁺): m/z 726.2 (M+H).

Step C

### 7-[5-(4-Dimethylaminomethyl-benzylcarbamoyl)-3-(4-fluoro-phenyl)-1-isopropyl-4-phenyl-1H-pyrrol-2-yl]-3,5-dihydroxy-heptanoic Acid Tert-Butyl Ester

To solution of 6-{2-[5-(4-dimethylaminomethyl-benzylcarbamoyl)-3-(4-fluoro-phenyl)-1-isopropyl-4-phenyl-1H-pyrrol-2-yl]-ethyl}-2,2-dimethyl-[1,3]dioxan-4-yl)-acetic acid tert-butyl ester (250 mg, 0.34 mmol) in MeOH (20 ml), 1.0 N HCl (2.0 ml) was added at 25° C. and the reaction was stirred for 4 hrs. The MeOH was partially removed and aqueous NaHCO₃ (saturated) and EtOAc (80 ml) were added. The organic phase was washed with brine, dried over Na₂SO₄, filtered and concentrated. The crude product was purified by silica gel chromatography (0-10% MeOH/DCM) to afford 7-[5-(4-dimethylaminomethyl-benzylcarbamoyl)-3-(4-fluoro-phenyl)-1-isopropyl-4-phenyl-1H-pyrrol-2-yl]-3,5-dihydroxy-heptanoic acid tert-butyl ester (164 mg, 70%):MS(APCI⁺): m/z 686.2 (M+H).

Step D

### (3R,5R)-7-[5-(4-Dimethylaminomethyl-benzylcarbamoyl)-3-(4-fluoro-phenyl)-1-isopropyl-4-phenyl-1H-pyrrol-2-yl]-3,5-dihydroxy-heptanoic Acid Sodium Salt

To a solution of 7-[5-(4-dimethylaminomethyl-benzylcarbamoyl)-3-(4-fluoro-phenyl)-1-isopropyl-4-phenyl-1H-pyrrol-2-yl]-3,5-dihydroxy-heptanoic acid tert-butyl ester (164 mg, 0.25 mmol) in MeOH (20 ml) at 25° C. was added aqueous NaOH solution (1.028 N, 0.26 ml). The reaction mixture stirred for 48 hrs after which time the reaction solvent was removed under reduced pressure. The resulting solid was azeotroped with toluene (3×25 ml), triturated with diethyl ether and dried under vacuum at 60° C. for overnight to afford desired 7-[5-(4-dimethylaminomethyl-benzylcarbamoyl)-3-(4-fluoro-phenyl)-1-isopropyl-4-phenyl-1H-pyrrol-2-yl]-3,5-dihydroxy-heptanoic acid sodium salt as light yellow solid (120 mg, 73%): MS(APCI⁺): m/z 630.3 (M+H); H-NMR (DMSO-d₆) δ 8.32 (t, 1H), 7.05-6.82 (m, 13H), 5.69 (s, 1H), 4.78 (s, 1H), 4.55-4.41 (m, 1H), 4.12 (d, 2H), 3.71-3.63 (m, 1H), 3.55-3.45(m, 1H), 3.24 (s, 1H), 3.11 (s, 1H), 2.68-2.57(m, 1H), 2.48-2.42 (m, 1H), 2.04 (s, 6H), 1.94 (dd, 1H), 1.74 (dd, 1H), 1.60-1.24 (m, 2H), 1.45 (d, 6H), 1.23-1.16 (m, 1H), 0.85-0.75 (m, 1H).

### Example 56

### (3R,5R)-7-[5-(3-Aminomethyl-benzylcarbamoyl)-3-(4-fluoro-phenyl)-1-isopropyl-4-phenyl-1H-pyrrol-2-yl]-3,5-dihydroxy-heptanoic Acid Sodium Salt

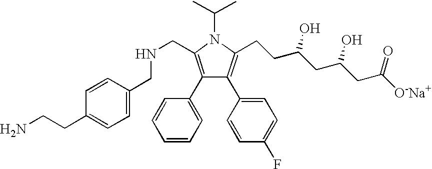 Step A

### (6-{2-[5-(3-Azidomethyl-benzylcarbamoyl)-3-(4-fluoro-phenyl)-1-isopropyl-4-phenyl-1H-pyrrol-2-yl]-ethyl}-2,2-dimethyl-[1,3]dioxan-4-yl)-acetic Acid Tert-Butyl Ester

To a solution of (6-{2-[3-(4-fluoro-phenyl)-1-isopropyl-5-(3-methanesulfonyloxymethyl-benzylcarbamoyl)-4-phenyl-1H-pyrrol-2-yl]-ethyl)-2,2-dimethyl-[1,3]dioxan-4-yl)-acetic acid tert-butyl ester (540 mg, 0.70 mmol) in DMF (15 ml) was added NaN₃ (0.452 g, 7.0 mmol) at room temperature. The reaction mixture was stirred at 50° C. for overnight after which time reaction mixture was concentrated under vacuum. The residue obtained was dissolved in EtOAc (150 ml), the organic solution was washed with H₂O and brine, dried over Na₂SO₄, filtered, and concentrated under vacuum. The crude product was purified by silica gel chromatography (5-30% EtOAc/Hexane) to afford (6-{2-[5-(3-azidomethyl-benzylcarbamoyl)-3-(4-fluoro-phenyl)-1-isopropyl-4-phenyl-1H-pyrrol-2-yl]-ethyl}-2,2-dimethyl-[1,3]dioxan-4-yl)-acetic acid tert-butyl ester (0. 50g, 98%): MS(APCI⁺): m/z 724.3 (M+H).

Step B

### 7-[5-(3-Azidomethyl-benzylcarbamoyl)-1-ethyl-3-(4-fluoro-phenyl)-4-phenyl-1H-pyrrol-2-yl]-3,5-dihydroxy-heptanoic Acid Isopropyl Ester

To a solution of (6-{2-[5-(3-azidomethyl-benzylcarbamoyl)-3-(4-fluoro-phenyl)-1-isopropyl-4-phenyl-1H-pyrrol-2-yl]-ethyl}-2,2-dimethyl-[1,3]dioxan-4-yl)-acetic acid tert-butyl ester (0.50 g, 0.69 mmol) in MeOH at 25° C. was added 1.0 N HCl (5 ml). The resulting mixture was stirred for 2 hr at 25° C. MeOH was then partially removed and the remaining solution was neutralized with aqueous NaHCO₃ (saturated) and diluted with EtOAc (100 ml). The organic phase was washed with brine, dried over Na₂SO₄, filtered and concentrated. The crude product was purified by silica gel chromatography (20-50% EtOAc/Hexane) to provide 7-[5-(3-azidomethyl-benzylcarbamoyl)-1-ethyl-3-(4-fluoro-phenyl)-4-phenyl-1H-pyrrol-2-yl]-3,5-dihydroxy-heptanoic acid isopropyl ester (0.38 g, 80%): MS(APCI⁺): m/z 684.4 (M+H);

Step C

### 7-[5-(3-Aminomethyl-benzylcarbamoyl)-3-(4-fluoro-phenyl)-1-isopropyl-4-phenyl-1H-pyrrol-2-yl]-3,5-dihydroxy-heptanoic Acid Tert-Butyl Ester

To a solution of 7-[5-(3-azidomethyl-benzylcarbamoyl)-1-ethyl-3-(4-fluoro-phenyl)-4-phenyl-1H-pyrrol-2-yl]-3,5-dihydroxy-heptanoic acid isopropyl ester (365 mg, 0.54 mmol) in MeOH (50 ml) was added Lindlar's catalyst (100 mg). Reaction vessel was evacuated and charged with H₂ (4295 psi/mole). The reaction mixture was stirred for 16 hr after which catalyst was filtered off. Filtrate was concentrated. The crude product was purified by silica gel chromatography (8% MeOH in DCM, 1% NH₄OH) to afford desired 7-[5-(3-aminomethyl-benzylcarbamoyl)-3-(4-fluoro-phenyl)-1-isopropyl-4-phenyl-1H-pyrrol-2-yl]-3,5-dihydroxy-heptanoic acid tert-butyl ester (320 mg, 91%): MS(APCI⁺): m/z 658.4 (M+H).

Step D

### (3R,5R)-7-[5-(3-Aminomethyl-benzylcarbamoyl)-3-(4-fluoro-phenyl)-1-isopropyl-4-phenyl-1 H-pyrrol-2-yl]-3,5-dihydroxy-heptanoic Acid Sodium Salt

To a solution of 7-[5-(3-aminomethyl-benzylcarbamoyl)-3-(4-fluoro-phenyl)-1-isopropyl-4-phenyl-1H-pyrrol-2-yl]-3,5-dihydroxy-heptanoic acid tert-butyl ester (320 mg, 0.49 mmol) in MeOH (15 ml) was added aqueous NaOH solution (511 ul, 0.52 mmol; 1.028 N). The reaction mixture stirred for 48 hr after which time the reaction solvent was removed under reduced pressure. The resulting solid was azeotroped with toluene (3×25 ml), triturated with diethyl ether and dried under vacuum at 60° C. for overnight to afford desired 7-[5-(3-Aminomethyl-benzylcarbamoyl)-3-(4-fluoro-phenyl)-1-isopropyl-4-phenyl-1H-pyrrol-2-yl]-3,5-dihydroxy-heptanoic acid sodium salt (298 mg, 98%): MS(APCI⁺): m/z 602.4 (M+H); H-NMR (DMSO-d₆) δ 8.32 (t, 1H), 7.05-6.82 (m, 13H), 4.74 (s, 1H), 4.49 (s, 1H), 4.12 (s, 2H), 3.97 (s, 1H), 3.67 (s, 1H), 3.54 (s, 2H), 3.22 (s, 2H), 2.75-2.55 (m, 1H), 2.65-2.38 (m, 1H), 1.99-1.85(m, 1H), 1.78-1.63 (m, 1H), 1.59-1.08 (m, 10H).

### Example 57

### (3R,5R)-7-[3-(4-Fluoro-phenyl)-5-(3-hydroxymethyl-benzylcarbamoyl)-1-isopropyl-4-phenyl-1H-pyrrol-2-yl]-3,5-dihydroxy-heptanoic Acid Sodium Salt

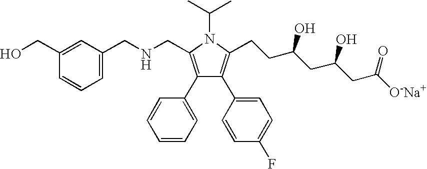

Prepared using the method described in Example 54:MS(APCI⁺): m/z 603.3 (M+H); H-NMR (DMSO-d₆) δ 8.33 (t, 1H), 7.20-6.79 (m, 13H), 5.08 (s, 1H), 4.75 (s, 1H), 4.57-4.42 (m, 1H), 4.36 (s, 2H), 4.12 (d, 2H), 3.73-3.60 (m, 1H), 3.55-3.40 (m, 1H), 2.72-2.57 (m, 1H), 2.54-2.39 (m, 1H), 2.24 (s, 1H), 1.95 (dd, 1H), 1.74(dd, 1H), 1.61-1.26(m, 2H), 1.44(d, 6H), 1.24-1.18(m, 1H),0.88-0.78 (m, 1H).

### Example 58

### (3R,5R)-7-[5-(3-Dimethylaminomethyl-benzylcarbamoyl)-3-(4-fluoro-phenyl)-1-isopropyl-4-phenyl-1H-pyrrol-2-yl]-3,5-dihydroxy-heptanoic Acid

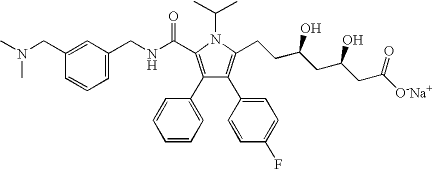

Prepared using the method described in Example 55: MS(APCI⁺): nz/z 630.3 (M+H); H-NMR (DMSO-d₆) δ 8.36 (t, 1H), 7.58 (s, 1H), 7.05-6.82 (m, 13H), 4.75 (s, 1H), 4.50-4.46 (m, 1H), 4.12 (d, 2H), 3.71-3.63 (m, 1H), 3.55-3.45(m, 1H), 3.27 (s, 1H), 3.21 (s, 1H), 2.68-2.57(m, 1H), 2.48-2.42 (m, 1H), 2.04 (s, 6H), 1.93 (dd, 1H), 1.72 (dd, 1H), 1.60-1.24 (m, 2H), 1.43 (d, 6H), 1.23-1.16 (m, 1H), 1.05 (s, 1H).

### Example 59

### (3R,5R)-7-[5-(4-Aminomethyl-benzylcarbamoyl)-3-(4-fluoro-phenyl)-1-isopropyl-4-phenyl-1H-pyrrol-2-yl]-3,5-dihydroxy-heptanoic Acid Sodium Salt

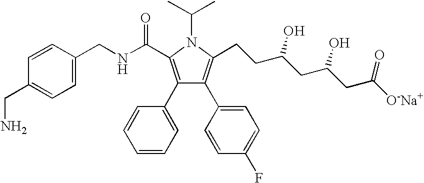

Prepared using the method described in Example 56: MS(APCI⁺): m/z 602.3 (M+H); H-NMR (DMSO-d₆) δ 8.27 (t, 1H), 7.05-6.82 (m, 13H), 4.74 (s, 1H), 4.48 (s, 1H), 4.11 (s, 2H), 3.97 (s, 1H), 3.67 (s, 1H), 3.54 (s, 2H), 3.22 (s, 2H), 2.75-2.55 (m, 1H), 2.65-2.38 (m, 1H), 1.99-1.85(m, 1H), 1.78-1.63 (m, 1H), 1.59-1.08 (m, 10H).

### Example 60

### (3R,5R)-7-[5-(4-Azidomethyl-benzylcarbamoyl)-3-(4-fluoro-phenyl)-1-isopropyl-4-phenyl-1H-pyrrol-2-yl]-3,5-dihydroxy-heptanoic Acid Sodium Salt

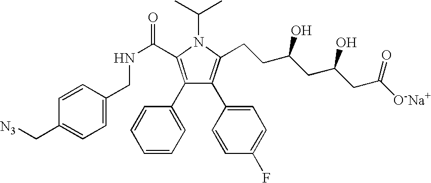

Prepared using the method described in Example 56: MS(APCI⁺): m/z 628.3 (M+H); H-NMR (DMSO-d₆) δ 8.37 (t, 1H), 7.38-6.82 (m, 13H), 4.75 (s, 1H), 4.57-4.41(m, 1H), 4.31 (s, 2H), 4.15 (d, 2H), 3.68 (s, 1H), 3.54 (s, 1H), 3.18-3.11 (m, 1H), 2.70-2.55 (m, 1H), 2.44-2.38 (m, 1H), 2.01-1.88 (m, 1H), 1.81-1.63 (m, 1H), 1.60-0.75 (m, 4H), 1.44 (d, 6H).

### Example 61

### (3R,5R)-7-[3-(4-fluoro-phenyl)-1-isopropyl-5-phenylcarbamoyl-4-pyridin-2-yl-1H-pyrrol-2-yl]-3,5-dihydroxy-heptanoic Acid

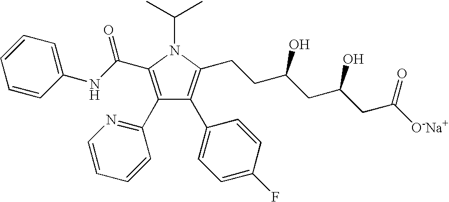 Step A

### 3-(4-Fluoro-phenyl)-2-pyridin-2-yl-acrylonitrile

To a solution of 4-fluorobenzaldehyde (52.5 g, 423 mmol) in EtOH (200 mL) at 25° C. was added pyridin-2-yl-acetonitrile (50.0 g, 423 mmol) and NaOEt (151 g of 21% solution, 466 mmol). The reaction was stirred at 25° C. for 0.5 hr during which time a light brown precipitate developed. The solid was isolated by filtration and washed with EtOH (75 mL). The product was then dried under vacuum to afford 3-(4-fluoro-phenyl)-2-pyridin-2-yl-acrylonitrile (87 g, 92%) which was used without further purification: MS(APCI⁺): m/z 225.3 (M+H); H-NMR (CDCl₃) δ 8.62 (d, 1H), 8.40 (s, 1H), 8.06-8.01 (m, 2H), 7.92-7.88 (m, 1H), 7.80-7.78 (d, 1H), 7.41-7.33 (m, 3H).

Step B

### 3-(4-Fluoro-phenyl)-4-pyridin-2-yl-1H-pyrrole-2-carboxylic Acid Ethyl Ester

A solution of 3-(4-fluoro-phenyl)-2-pyridin-2-yl-acrylonitrile (25.0 g, 112 mmol) and ethyl isocyanoacetate (12.3 mL, 112 mmol) in THF (300 mL) was slowly added to a solution of KOtBu (223 mL of 1.0 M solution, 223 mmol) in THF (100 mL) at 0° C. The resulting reaction mixture was stirred at 0° C. for 1.5 hr after which time TLC indicated that the reaction was complete. The reaction was transferred to a separatory funnel and ethyl acetate (500 mL) and water (200 mL) were added. The organic layer was separated and washed with brine and dried over Na₂SO₄. Upon concentration of the organic layer, the crude product solidified to give 3-(4-fluoro-phenyl)-4-pyridin-2-yl-1H-pyrrole-2-carboxylic acid ethyl ester (30.5 g, 88%) as a brown solid which was utilized without further purification: MS(APCI⁺): m/z 311.1 (M+H);

Step C

### 3-(4-Fluoro-phenyl)-1-isopropyl-4-pyridin-2-yl-1H-pyrrole-2-carboxylic Acid Ethyl Ester

To a solution of 3-(4-fluoro-phenyl)-4-pyridin-2-yl-1H-pyrrole-2-carboxylic acid ethyl ester (30.5 g, 98.3 mmol) in DMSO (100 mL) at 25° C. was added powdered KOH (24.8 g, 442 mmol) and the reaction mixture was stirred at 25° C. for 0.5 hr. Subsequently, 2-iodopropane (26.5 mL, 265 mmol) was added dropwise to the suspension and the reaction was stirred for an additional 0.5 hr at 25° C. Ether (300 mL) and water (100 mL) were then added and the organic layer was separated, dried (Na2SO4) and concentrated to a crude oil which was purified by silica gel chromatography (10-40% EtOAc/Hexane) to give 3-(4-fluoro-phenyl)-1-isopropyl-4-pyridin-2-yl-1H-pyrrole-2-carboxylic acid ethyl ester (23.2 g, 74%): MS(APCI⁺): m/z 353.3 (M+H); H-NMR (DMSO-d₆) δ 8.42 (d, 1H), 7.79 (s, 1H), 7.47-7.43 (m, 1H), 7.20-7.04 (m, 5H), 6.66-6.63 (m, 1H), 5.28-5.25 (m, 1H), 3.91 (q, 2H), 1.46 (d, 6H), 0.81 (t, 3H).

Step D

### [3-(4-Fluoro-phenyl)-1-isopropyl-4-pyridin-2-yl-1H-pyrrol-2-yl]-methanol

To a solution of 3-(4-fluoro-phenyl)-1-isopropyl-4-pyridin-2-yl-1H-pyrrole-2-carboxylic acid ethyl ester (6.50 g, 18.4 mmol) in THF (120 mL) at −10° C. was slowly added lithium aluminum hydride (46.1 mL of 1.0 M in Et₂O, 46.1 mmol). The reaction was stirred at −10° C. for 1 hr after which time it was carefully quenched by slow addition of saturated NH₄Cl. Once the quench was complete, water was slowly added and the reaction mixture was extracted with ethyl acetate.

The organic layer was dried over Na2SO4 and concentrated. The product was purified by silica gel chromatography (50-75% EtOAc/Hexane) to afford [3-(4-fluoro-phenyl)-1-isopropyl-4-pyridin-2-yl-1H-pyrrol-2-yl]-methanol (5.54 g, 97%) as a white solid: MS(APCI⁺): m/z 311.1 (M+H); H-NMR (CDCl₃) [18.47 (d, 1H), 7.37 (bs, 1H), 7.30 (t, 1H), 7.22-7.17 (m, 3H), 7.04-6.92 (m, 3H), 6.69 (d, 1H), 4.62-4.57 (m, 1H), 4.49-4.48 (m, 2H), 1.51 (d, 6H).

Step E

### [3-(4-Fluoro-phenyl)-1-isopropyl-4-pyridin-2-yl-1H-pyrrol-2-ylmethyl]-triphenyl-phosphonium Bromide

To a solution of [3-(4-fluoro-phenyl)-1-isopropyl-4-pyridin-2-yl-1H-pyrrol-2-yl]-methanol (3.15 g, 10.1 mmol) in DCM (150 mL) was added triphenylphosphine hydrobromide (3.48 g, 10.2 mmol) and HCl (5.1 mL of 2.0 M solution in Et₂O, 10.1 mmol). The reaction was stirred at 25° C. for 1 hr after which time all starting material was consumed as determined by TLC. The organic layer was then washed with saturated NaHCO₃ and dried over Na2SO4. The organic layer was then concentrated to afford Reaction mixture was then evaporated under reduced pressure and dried under high vacuum for 12 hr to afford [3-(4-fluoro-phenyl)-1-isopropyl-4-pyridin-2-yl-1H-pyrrol-2-ylmethyl]-triphenyl-phosphonium bromide (6.36 g, 99%) as a yellow solid of sufficient purity for use in the next step.

Step F

### (6-{2-[3-(4-Fluoro-phenyl)-1-isopropyl-4-pyridin-2-yl-1H-pyrrol-2-yl]-vinyl}-2,2-dimethyl-[1,3]dioxan-4-yl)-acetic Acid Tert-Butyl Ester

To a solution of [3-(4-fluoro-phenyl)-1-isopropyl-4-pyridin-2-yl-1H-pyrrol-2-ylmethyl]-triphenyl-phosphonium bromide (6.00 g, 8.93 mmol) in THF:DMSO (500 mL, 25:1) at −78° C. was added NaHMDS (9.12 mL of a 1.0 M solution in THF, 9.12 mmol). An orange color was noted as the base was added to the reaction mixture. The reaction was stirred at −78° C. for 5 min after which time a solution of (6-Formyl-2,2-dimethyl-[1,3]dioxan-4-yl)-acetic acid tert-butyl ester (2.16 g, 8.35 mmol) in THF (20 mL) was added. The reaction mixture was stirred at −78° C. for 0.5 hr and then allowed to warm to 25° C. over 1.5 hr. The reaction was quenched by addition of saturated NH₄Cl. Ethyl acetate was then added and organic layer was washed with water, dried (Na₂SO₄), concentrated. The resulting oil was purified by silica gel chromatography (20-25% EtOAc/Hexane) to provide (6-{2-[3-(4-fluoro-phenyl)-1-isopropyl-4-pyridin-2-yl-1H-pyrrol-2-yl]-vinyl}-2,2-dimethyl-[1,3]dioxan-4-yl)-acetic acid tert-butyl ester (2.69 g, 66%) as a mixture of cis/trans isomers: MS(APCI⁺): m/z 535.3 (M+H);

Step G

### (6-{2-[3-(4-Fluoro-phenyl)-1-isopropyl-4-pyridin-2-yl-1H-pyrrol-2-yl]-ethyl}-2,2-dimethyl-[1,3]dioxan-4-yl)-acetic Acid Tert-Butyl Ester

To a solution of (6-{2-[3-(4-fluoro-phenyl)-1-isopropyl-4-pyridin-2-yl-1H-pyrrol-2-yl]-vinyl)-2,2-dimethyl-[1,3]dioxan-4-yl)-acetic acid tert-butyl ester (3.11 g, 5.82 mmol) in MeOH (100 mL) was added 10% Pd/C (300 mg). The reaction vessel was then evacuated and treated with hydrogen (50 psi) for 12 hr at 25° C. The reaction mixture was then filtered through a pad of celite and the filtrate was concentrated. The resulting oil was purified by silica gel chromatography (30-50% EtOAc/Hex ane) to provide (6-{2-[3-(4-fluoro-phenyl)-1-isopropyl-4-pyridin-2-yl-1H-pyrrol-2-yl]-ethyl}-2,2-dimethyl-[1,3]dioxan-4-yl)-acetic acid tert-butyl ester (1.65 g, 53%): MS(APCI⁺): m/z 537.7 (M+H).

Step H

### (6-{2-[3-(4-Fluoro-phenyl)-5-iodo-1-isopropyl-4-pyridin-2-yl-1H-pyrrol-2-yl]-ethyl}-2,2-dimethyl-[1,3]dioxan-4-yl)-acetic Acid Tert-Butyl Ester

To a solution of (6-{2-[3-(4-fluoro-phenyl)-1-isopropyl-4-pyridin-2-yl-1H-pyrrol-2-yl]-ethyl}-2,2-dimethyl-[1,3]dioxan-4-yl)-acetic acid tert-butyl ester (0.80 g, 1.49 mmol) in DMF (8 mL) at 25° C. was added N-iodosuccinimide (0.309 g, 1.79 mmol). The reaction was stirred at 25° C. for 1.5 hours after which time DCM (50 mL) and saturated NaHCO₃ (50 mL) were then added and the organic layer was separated, washed with brine and dried over Na₂SO₄. The organic layer was concentrated and the product was purified by silica gel chromatography (10% EtOAc/Hexane) to give (6-{2-[3-(4-fluoro-phenyl)-5-iodo-1-isopropyl-4-pyridin-2-yl-1H-pyrrol-2-yl]-ethyl}-2,2-dimethyl-[1,3]dioxan-4-yl)-acetic acid tert-butyl ester (0.912 g, 92%): MS(APCI⁺): m/z 663.1 (M+H).

Step I

### (3R,5R)-7-[3-(4-Fluoro-phenyl)-1-isopropyl-5-phenylcarbamoyl-4-pyridin-2-yl-1H-pyrrol-2-yl]-3,5-dihydroxy-heptanoic Acid Tert-Butyl Ester

A high pressure reactor was charged with (6-{2-[3-(4-fluoro-phenyl)-5-iodo-1-isopropyl-4-pyridin-2-yl-1H-pyrrol-2-yl]-ethyl}-2,2-dimethyl-[1,3]dioxan-4-yl)-acetic acid tert-butyl ester (0.735 g, 1.11 mmol), Pd(PPh₃)₂Cl₂ (0.200 g), aniline (516 mg, 5.55 mmol) and toluene (35 ml). The reactor was pressurized with CO (400 psi) and heated to 100° C. for 15 hr. After cooling to 25° C., the reaction solvent was removed under reduced pressure and the resulting residue was purified by silica gel chromatography to give 7-[3-(4-Fluoro-phenyl)-1-isopropyl-5-phenylcarbamoyl-4-pyridin-2-yl-1H-pyrrol-2-yl]-3,5-dihydroxy-heptanoic acid tert-butyl ester (0.044 g, 6%). Note that acetonide protecting group was cleaved under these reaction conditions: MS(APCI⁺): m/z 616.2 (M+H).

Step J

### (3R,5R)-7-[3-(4-fluoro-phenyl)-1-isopropyl-5-phenylcarbamoyl-4-pyridin-2-yl-1H-pyrrol-2-yl]-3,5-dihydroxy-heptanoic Acid Sodium Salt

To a solution of 7-[3-(4-Fluoro-phenyl)-1-isopropyl-5-phenylcarbamoyl-4-pyridin-2-yl-1H-pyrrol-2-yl]-3,5-dihydroxy-heptanoic acid tert-butyl ester (0.044 g, 0.0715 mmol) in MeOH (3 mL) was added 1.02 N NaOH (0.0730 mL) and the reaction was stirred at 25° C. for 72 hr. The reaction mixture was then concentrated and azeotroped with toluene (25 mL×3). The product was dried under vacuum at 60° C. to give (3R,5R)-7-[3-(4-fluoro-phenyl)-1-isopropyl-5-phenylcarbamoyl-4-pyridin-2-yl-1H-pyrrol-2-yl]-3,5-dihydroxy-heptanoic acid sodium salt (0.039 g, 94%) as a light yellow solid: MS(APCI⁺): m/z 560.2 (M+H); H-NMR (DMSO-d₆) δ 10.3 (s, 1H), 8.35-8.34 (m, 1H), 7.68 (s, 1H), 7.44-7.39 (m, 3H), 7.24-7.14 (m, 2H), 7.06-6.93 (m, 5H), 6.79 (d, 1H), 4.77 (bs, 1H), 4.67-4.61 (m, 1H), 3.66-3.60 (m, 1H), 3.58-3.53 (m, 1H), 2.70-2.61 (m, 1H), 2.41-2.47 (m, 1H), 1.97-1.90 (m, 1H), 1.74-1.68 (m, 1H), 1.55-1.18 (m, 10H).

### Example 62

### (3R,5R)-7-[5-carbamoyl-3-(4-fluoro-phenyl)-1-isopropyl-4-pyridin-2-yl-1H-pyrrol-2-yl]-3,5-dihydroxy-heptanoic Acid Sodium Salt

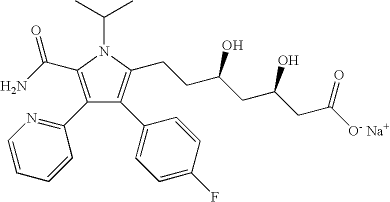 Step A

### (6-{2-[5-Carbamoyl-3-(4-fluoro-phenyl)-1-isopropyl-4-pyridin-2-yl-1H-pyrrol-2-yl]-ethyl}-2,2-dimethyl-[1,3]dioxan-4-yl)-acetic Acid Tert-Butyl Ester

A high pressure reactor was charged with (6-{2-[3-(4-fluoro-phenyl)-5-iodo-1-isopropyl-4-pyridin-2-yl-1H-pyrrol-2-yl]-ethyl}-2,2-dimethyl-[1,3]dioxan-4-yl)-acetic acid tert-butyl ester (0.465 g, 0.702 mmol), Pd(PPh₃)₂Cl₂ (0.064 g) and toluene (25 ml). The reactor was pressurized with ammonia (85 (psi) and CO (400 psi) and then heated to 100° C. for 15 hr. After cooling to 25° C., the reaction solvent was removed under reduced pressure and the resulting residue was purified by silica gel chromatography to give (6-{2-[5-carbamoyl-3-(4-fluoro-phenyl)-1-isopropyl-4-pyridin-2-yl-1H-pyrrol-2-yl]-ethyl}-2,2-dimethyl-[1,3]dioxan-4-yl)-acetic acid tert-butyl ester (0.103 g, 25%): MS(APCI⁺): m/z 580.3 (M+H);

Step B

### (3R,5R)-7-[5-Carbamoyl-3-(4-fluoro-phenyl)-1-isopropyl-4-pyridin-2-yl-1H-pyrrol-2-yl]-3,5-dihydroxy-heptanoic Acid Tert-Butyl Ester

To a solution of (6-{2-[5-carbamoyl-3-(4-fluoro-phenyl)-1-isopropyl-4-pyridin-2-yl-1H-pyrrol-2-yl]-ethyl}-2,2-dimethyl-[1,3]dioxan-4-yl)-acetic acid tert-butyl ester (0.103 g, 0.178 mmol) in MeOH (5 mL) at 25° C. was added 1 N HCl (0.533 mL, 0.533 mmol). The reaction was stirred at 25° C. for 2 hr after which time the solvent was removed by evaporation and ethyl acetate (20 mL) was added. The organic layer was washed with saturated NaHCO₃, water and brine prior to drying over Na₂SO4. After concentration, the product was purified by silica gel chromatography (50-100% EtOAc/Hexane) to give (6-{2-[5-carbamoyl-3-(4-fluoro-phenyl)-1-isopropyl-4-pyridin-2-yl-1H-pyrrol-2-yl]-ethyl}-2,2-dimethyl-[1,3]dioxan-4-yl)-acetic acid tert-butyl ester (0.057 g, 59%) MS(APCI⁺): m/z 540.3 (M+H);

Step C

### (3R,5R)-7-[5-carbamoyl-3-(4-fluoro-phenyl)-1-isopropyl-4-pyridin-2-yl-1H-pyrrol-2-yl]-3,5-dihydroxy-heptanoic Acid Sodium Salt

To a solution of (6-{2-[5-carbamoyl-3-(4-fluoro-phenyl)-1-isopropyl-4-pyridin-2-yl-1H-pyrrol-2-yl]-ethyl)-2,2-dimethyl-[1,3]dioxan-4-yl)-acetic acid tert-butyl ester (0.057 g, 0.106 mmol) in MeOH (5 mL) was added 1.02 N NaOH (0.108 mL, 0.111 mmol) and the reaction was stirred at 25° C. for 72 hr. The reaction mixture was then concentrated and azeotroped with-toluene (25 mL×3). The product was dried under vacuum at 60° C. to give (3R,5R)-7-[5-carbamoyl-3-(4-fluoro-phenyl)-1-isopropyl-4-pyridin-2-yl-1H-pyrrol-2-yl]-3,5-dihydroxy-heptanoic acid sodium salt (0.051 g, 96%): MS(APCI⁺): m/z 484.2 (M+H); H-NMR (DMSO-d₆) δ 8.41-8.39 (m, 1H), 7.59-7.44 (m, 3H), 7.11-6.83 (m, 6H), −4.74 (bs, 1H), 4.65-4.62 (m, 1H), 3.66-3.62 (m, 1H), 3.51-3.47 (m, 1H), 2.66-2.62 (m, 1H), 2.49-2.43 (m, 1H), 1.95-1.91 (m, 1H), 1.75-1.69 (m, 1H), 1.53-1.14(m, 10H).

### Example 63

### 3R,5R)-7-[5-cyano-3-(4-fluoro-phenyl)-1-isopropyl-4-pyridin-2-yl-1H-pyrrol-2-yl]-3,5-dihydroxy-heptanoic Acid Sodium Salt

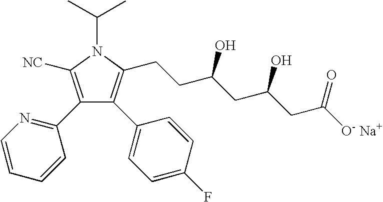 Step A

### (6-{2-[5-Cyano-3-(4-fluoro-phenyl)-1-isopropyl-4-pyridin-2-yl-1H-pyrrol-2-yl]-ethyl)-2,2-dimethyl-[1,3]dioxan-4-yl)-acetic Acid Tert-Butyl Ester

To a solution of (6-{2-[3-(4-fluoro-phenyl)-5-iodo-1-isopropyl-4-pyridin-2-yl-1H-pyrrol-2-yl]-ethyl}-2,2-dimethyl-[1,3]dioxan-4-yl)-acetic acid tert-butyl ester in DMF (10 mL) was added CuCN (0.153 g, 1.71 mmol) and KCN (0.111 g, 1.71 mmol). The reaction was heated to 120° C. for 2 hrs. After cooling to 25° C., the reaction solvent was removed under reduced pressure and the resulting residue was purified by silica gel chromatography (20-50% EtOAc/Hexane) to give (6-{2-[5-cyano-3-(4-fluoro-phenyl)-1-isopropyl-4-pyridin-2-yl-1H-pyrrol-2-yl]-ethyl}-2,2-dimethyl-[1,3]dioxan-4-yl)-acetic acid tert-butyl ester (0.512 g, 64%): MS(APCI⁺): m/z 562.3 (M+H).

Step B

### (3R,5R)-7-[5-Cyano-3-(4-fluoro-phenyl)-1-isopropyl-4-pyridin-2-yl-1H-pyrrol-2-yl]-3,5-dihydroxy-heptanoic Acid Tert-Butyl Ester

To a solution of (6-{2-[5-cyano-3-(4-fluoro-phenyl)-1-isopropyl-4-pyridin-2-yl-1H-pyrrol-2-yl]-ethyl-2,2-dimethyl-[1,3]dioxan-4-yl)-acetic acid tert-butyl ester (0.182 g, 0.324 mmol) in MeOH (10 mL) at 25° C. was added 1 N HCl (1.62 mL, 1.62 mmol). The reaction was stirred at 25° C. for 2 hr after which time the solvent was removed by evaporation and ethyl acetate (20 mL) was added. The organic layer was washed with saturated NaHCO₃, water and brine prior to drying over Na₂SO4. After concentration, the product was purified by silica gel chromatography (50% EtOAc/Hexane) to give (3R,5R)-7-[5-Cyano-3-(4-fluoro-phenyl)-1-isopropyl-4-pyridin-2-yl-1H-pyrrol-2-yl]-3,5-dihydroxy-heptanoic acid tert-butyl ester (0.082 g, 49%): MS(APCI⁺): m/z 522.2 (M+H);

Step C

### (3R,5R)-7-[5-cyano-3-(4-fluoro-phenyl)-1-isopropyl-4-pyridin-2-yl-1H-pyrrol-2-yl]-3,5-dihydroxy-heptanoic Acid Sodium Salt

To a solution of 7-[5-Cyano-3-(4-fluoro-phenyl)-1-isopropyl-4-pyridin-2-yl-1H-pyrrol-2-yl]-3,5-dihydroxy-heptanoic acid tert-butyl ester (0.081 g, 0.155 mmol) in MeOH (10 mL) was added 1.03 N NaOH (0.159 mL, 0.163 mmol) and the reaction was stirred at 25° C. for 48 hr. The reaction mixture was then concentrated and azeotroped with toluene (25 mL×3). The product was dried under vacuum at 60° C. to give (3R,5R)-7-[5-cyano-3-(4-fluoro-phenyl)-1-isopropyl-4-pyridin-2-yl-1H-pyrrol-2-yl]-3,5-dihydroxy-heptanoic acid sodium salt (0.069 g, 91%): MS(APCI⁺): m/z 466.3 (M+H); H-NMR (DMSO-d₆) δ 8.49-8.48 (m, 1H), 7.58-7.49 (m, 2H), 7.19-7.15 (m, 1H), 7.12-7.07 (m, 3H), 6.85-6.83 (m, 1H), 4.79 (bs, 1H), 4.69-4.65 (m, 1H), 3.67-3.65 (m, 1H), 3.27-3.21 (m, 1H), 2.65-2.61 (m, 1H), 2.47-2.42 (m, 1H), 1.95-1.91 (m, 1H), 1.75-1.70 (m, 1H), 1.58-1.16 (m, 10H).

### Example 64

### (3R,5R)-7-[3-(4-fluoro-phenyl)-1-isopropyl-4-pyridin-2-yl-1H-pyrrol-2-yl]-3,5-dihydroxy-heptanoic Acid Sodium Salt

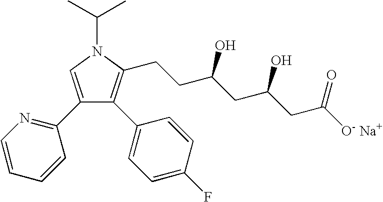 Step A

### (3R,5R)-7-[3-(4-Fluoro-phenyl)-1-isopropyl-4-pyridin-2-yl-1H-pyrrol-2-yl]-3,5-dihydroxy-heptanoic Acid Tert-Butyl Ester

To a solution of (6-{2-[3-(4-fluoro-phenyl)-1-isopropyl-4-pyridin-2-yl-1H-pyrrol-2-yl]-ethyl}-2,2-dimethyl-[1,3]dioxan-4-yl)-acetic acid tert-butyl ester [Example #35#, Step G] (0.205 g, 0.382 mmol) in MeOH (10 mL) at 25° C. was added 1 N HCl (1.91 mL, 1.91 mmol). The reaction was stirred at 25° C. for 2 hr after which time the solvent was removed by evaporation and ethyl acetate (20 mL) was added. The organic layer was washed with saturated NaHCO₃, water and brine prior to drying over Na₂SO4. After concentration, the product was purified by silica gel chromatography (50-60% EtOAc/Hexane) to give (3R,5R)-7-[3-(4-Fluoro-phenyl)-1-isopropyl-4-pyridin-2-yl-1H-pyrrol-2-yl]-3,5-dihydroxy-heptanoic acid tert-butyl ester (0.155 g, 82%): MS(APCI⁺): m/z 497.2 (M+H).

Step B

### (3R,5R)-7-[3-(4-fluoro-phenyl)-1-isopropyl-4-pyridin-2-yl-1H-pyrrol-2-yl]-3,5-dihydroxy-heptanoic Acid Sodium Salt

To a solution of 7-[3-(4-fluoro-phenyl)-1-isopropyl-4-pyridin-2-yl-1H-pyrrol-2-yl]-3,5-dihydroxy-heptanoic acid tert-butyl ester (0.135 g, 0.272 mmol) in MeOH (5 mL) was added 1.03 N NaOH (0.278 mL, 0.285 mmol) and the reaction was stirred at 25° C. for 24 hr. The reaction mixture was then concentrated and azeotroped with toluene (25 mL×3). The product was dried under vacuum at 60° C. to give (3R,5R)-7-[3-(4-fluoro-phenyl)-1-isopropyl-4-pyridin-2-yl-1H-pyrrol-2-yl]-3,5-dihydroxy-heptanoic acid sodium salt (0.105 g, 84%): MS(APCI⁺): m/z 441.2 (M+H); H-NMR (DMSO-d₆) δ 8.33-8.31 (m, 1H), 7.39-7.35 (m, 1H), 7.29 (s, 1H), 7.21-7.08 (m, 4H), 6.94-6.91 (m, 1H), 6.66-6.64 (d, 1H), 4.43-4.31 (m, 2H),

### Example 65

### (3R,5R)-7-[5-Benzylcarbamoyl-3-(4-fluoro-phenyl)-1-isopropyl-4-pyridin-2-yl-1H-pyrrol-2-yl]-3,5-dihydroxy-heptanoic Acid

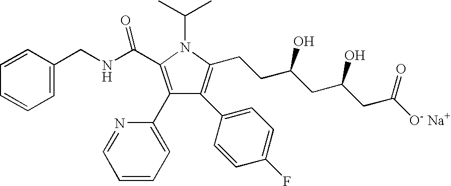

Prepared using the method described in Example 61. MS(APCI⁺): m/z 574.2 (M+H); H-NMR (DMSO-d₆) δ 8;71 (t, 1H), 8.24 (d, 1H), 7.55-6.77(m, 13H), 4.74 (s, 1H), 4.61-4.55 (m, 1H), 4.18 (d, 2H), 3.75-3.59 (m, 1H), 3.57-3.43 (m, 1H), 2.77-2.58 (m, 1H), 2.55-2.38 (m, 1H), 1.95 (dd, 1H), 1.73 (dd, 1H) 1.58-1.03 (m, 4), 1.48 (d, 6H).

### Example 66

### 7-[5-Ethylcarbamoyl-3-(4-fluoro-phenyl)-1-isopropyl-4-pyridin-2-yl-1H-pyrrol-2-yl]-3,5-dihydroxy-heptanoic Acid

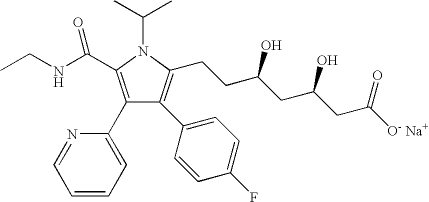

Prepared using the method described in Example 61. MS(APCI⁺): m/z 512.4 (M+H).

### Example 67

### (3R,5R)-7-[5-(3-dimethylcarbamoyl-phenylcarbamoyl)-3,4-bis-(4-fluoro-phenyl)-1-isopropyl-1H-pyrrol-yl]-3,5-dihydroxy-heptanoate Sodium Salt

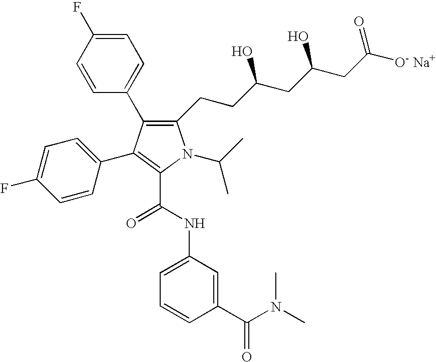 Step A

### 3,4-Bis-(4-fluoro-phenyl)-5-formyl-1-isopropyl-1H-pyrrole-2-carboxylic Acid (3-dimethylcarbamoyl-phenyl)-amide

To a mixture of 3,4-bis-(4-fluoro-phenyl)-5-formyl-1-isopropyl-1H-pyrrole-2-carboxylic acid prepared in Step G of Example 1 (3.0 g, 8.1 mmoles) in anhydrous dichloromethane (90 mL) was added 2 drops of anhydrous DMF, followed by oxalyl chloride (0.85 mL, 9.7 mmoles). The reaction mixture was stirred at room temperature for 18 hrs and then evaporated and dried to provide 3.15 g (100% crude) of a dark green tacky solid as the acid chloride. The solid was dissolved in anhydrous dichloromethane (50 mL) and then added dropwise to a cold (0° C.) mixture of 3-amino-N,N-dimethyl-benzamide (H. Wenker, JACS, 60: 1080 1938) (1.6 g, 9.7 mmoles) and diisopropylethylamine (1.8 mL, 11 mmoles) in anhydrous dichloromethane (50 mL). The reaction mixture was stirred at −5 to 0° C. for 2 hrs and then at room temperature for 18 hrs. The reaction mixture was diluted with a mixture of 300 mL of dichloromethane and 50 mL of water. The aqueous layer was separated and then the organic layer was washed with 1 N HCl (3×50 mL), 5% sodium bicarbonate (2×50 mL), and with brine (50 mL). The organic layer was separated, dried (sodium sulfate), filtered, and then evaporated to give a residue, which was purified by flash chromatographed (silica gel, 60% ethyl acetate in hexane) to provide 3.03 g (72%) of the desired product as a tan solid: mp 133-135° C.; MS(APCI⁺) m/z 516.

Step B

### Cis/trans-(3R)-3-(tert-butyl-dimethyl-silanyloxy)-7-[5-(3-dimethylcarbamoyl-phenylcarbamoyl)-3,4-bis-(4-fluoro-phenyl)-1-isopropyl-1H-pyrrol-yl]-5-oxo-hept-6-enoic Acid Methyl Ester

The title compound was prepared from 3,4-Bis-(4-fluoro-phenyl)-5-formyl-1-isopropyl-1H-pyrrole-2-carboxylic acid (3-dimethylcarbamoyl-phenyl)-amide by the method described in Step I of Example 1: mp 135-137° C.; MS(APCI⁺) m/z 772.

Step C

### Cis/trans-(3R)-7-[5-(3-dimethylcarbamoyl-phenylcarbamoyl)-3,4-bis-(4-fluoro-phenyl)-1-isopropyl-1H-pyrrol-yl]-3-hydroxy-5-oxo-hept-6-enoic Acid Methyl Ester

Method A

The title compound was prepared from cis/trans-(3R)-3-(tert-butyl-dimethyl-silanyloxy)-7-[5-(3-dimethylcarbamoyl-phenylcarbamoyl)-3,4-bis-(4-fluoro-phenyl)-1-isopropyl-1H-pyrrol-yl]-5-oxo-hept-6-enoic acid methyl ester by the method described in Step K of Example 2: mp 99-101° C.; MS(APCI⁺) m/z 658.

Method B

The title compound was prepared from cis/trans-(3R)-3-(tert-butyl-dimethyl-silanyloxy)-7-[5-(3-dimethylcarbamoyl-phenylcarbamoyl)-3,4-bis-(4-fluoro-phenyl)-1-isopropyl-1H-pyrrol-yl]-5-oxo-hept-6-enoic acid methyl ester by the method described in Step K of Example 1, substituting tetrahydrofuran for acetonitrile and 70% HF:pyridine for 48% HF:acetonitrile.

Step D

### trans-(3R,5S)-7-[5-(3-di Methylcarbamoyl-phenylcarbamoyl)-3,4-bis-(4-fluoro-phenyl)-1-isopropyl-1H-pyrrol-yl]-3,5-dihydroxy-hept-6-enoic Acid Methyl Ester and cis-(3R,5S)-7-[5-(3-dimethylcarbamoyl-phenylcarbamoyl)-3,4-bis-(4-fluoro-phenyl)-1-isopropyl-1H-pyrrol-yl]-3,5-dihydroxy-hept-6-enoic Acid Methyl Ester

The title compounds were prepared from cis/trans-(3R)-7-[5-(3-dimethylcarbamoyl-phenylcarbamoyl)-3,4-bis-(4-fluoro-phenyl)-1-isopropyl-1H-pyrrol-yl]-3-hydroxy-5-oxo-hept-6-enoic acid methyl ester by the method described in Step B of Example 2. cis isomer: mp 97-101° C.; MS(APCI⁺) m/z 642. trans isomer: 90-93° C.; MS(APCI⁺) m/z 642.

Step E

### (3R,5R)-7-[5-(3-dimethylcarbamoyl-phenylcarbamoyl)-3,4-bis-(4-fluoro-phenyl)-1-isopropyl-1H-pyrrol-yl]-3,5-dihydroxy-heptanoic Acid Methyl Ester

The title compound was prepared from cis/trans-(3R,5S)-7-[5-(3-dimethylcarbamoyl-phenylcarbamoyl)-3,4-bis-(4-fluoro-phenyl)-1-isopropyl-1H-pyrrol-yl]-3,5-dihydroxy-hept-6-enoic acid methyl ester by the method described in Step E of Example 4, substituting methanol for ethanol:tetrahydrofuran under hydrogen atmosphere at 50 psi: mp 95-98° C.; MS(APCI⁺) m/z 662.

Step F

### (3R,5R)-7-[5-(3-dimethylcarbamoyl-phenylcarbamoyl)-3,4-bis-(4-fluoro-phenyl)-1-isopropyl-1H-pyrrol-yl]-3,5-dihydroxy-heptanoate Sodium Salt

The title compound was prepared from (3R,5R)-7-[5-(3-dimethylcarbamoyl-phenylcarbamoyl)-3,4-bis-(4-fluoro-phenyl)-1-isopropyl-1H-pyrrol-yl]-3,5-dihydroxy-heptanoic acid methyl ester by the method described in Step M of Example 1: H¹ NMR (400 MHz DMSO-d₆) δ 10.09, 7.57, 7.40, 7.23, 7.08-6.84, 4.78, 4.58, 3.67, 3.55, 2.90, 2.80, 2.73-2.37, 1.94, 1.74, 1.62-1.29, 1.25-1.16; MS(APCI⁺) m/z 646.

### Example 68

### trans-(3R,5S)-7-[5-(3-dimethylcarbamoyl-phenylcarbamoyl)-3,4-bis-(4-fluoro-phenyl)-1-isopropyl-1H-pyrrol-yl]-3,5-dihydroxy-hept-6-enoate Sodium Salt

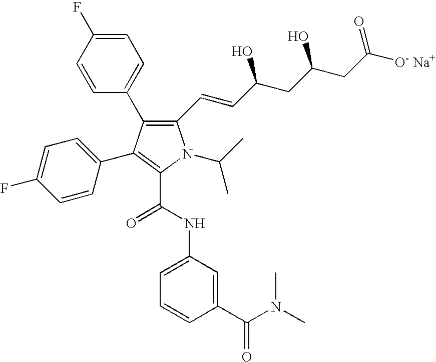

The title compound was prepared from trans-(3R,5S)-7-[5-(3-dimethylcarbamoyl-phenylcarbamoyl)-3,4-bis-(4-fluoro-phenyl)-1-isopropyl-1H-pyrrol-yl]-3,5-dihydroxy-hept-6-enoic acid methyl ester by the procedure described in Step C of Example 2: m.p: 210-214° C.; H¹NMR (400 MHz DMSO-d₆) δ 10.14, 7.41, 7.25, 7.05-6.85, 6.43, 5.37, 4.99, 4.66, 4.08, 3.49, 2.85, 1.91, 1.70, 1.55-1.45, 1.41-1.28, 1.11-0.95; MS(APCI⁺) m/z 628.

### Example 69

### (3R,5R)-7-{3,4-bis-(4-fluoro-phenyl)-1-isopropyl-5-[(pyridin-2-ylmethyl)-carbamoyl]-1H-pyrrol-2-yl}-3,5-dihydroxy-heptanoate Sodium Salt

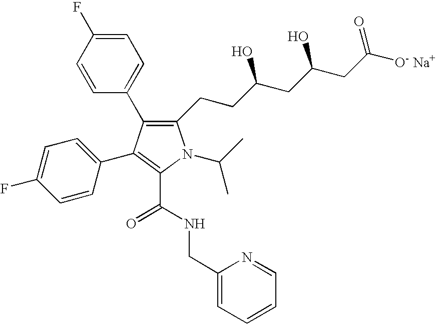 Step A

### 3,4-Bis-(4-fluoro-phenyl)-5-formyl-1-isopropyl-1H-pyrrole-2-carboxylic Acid (pyridine-2-ylmethyl)-amide

To a mixture of 3,4-bis-(4-fluoro-phenyl)-5-formyl-1-isopropyl-1H-pyrrole-2-carboxylic acid prepared in Step G of Example 1 (3.0 g, 8.1 mmoles,) in anhydrous dichloromethane (90 mL) was added 2 drops of anhydrous DMF, followed by oxalyl chloride (0.85 mL, 9.7 mmoles). The reaction mixture was stirred at room temperature for 18 hrs and then evaporated and dried to provide 3.15 g (100% crude) of a dark green tacky solid. The solid was dissolved in ethyl acetate (8 mL) and then added dropwise to a cold (−5° C.) mixture of 2-(aminomethyl)-pyridine (0.89 g, 8.3 mmoles) and sodium carbonate (1.3 g, 12 mmoles) in 4:1 ethyl acetate:water (40 mL). The reaction mixture was stirred at −5 to 0° C. for 21.5 hrs and then at room temperature for 18 hrs. The reaction mixture was filtered to collect a white solid, which was rinsed with ethyl acetate and then dried to provide 2.30 g of desired product. The filtrate above was diluted with ethyl acetate (300 mL) and then washed with saturated ammonium chloride (3×50 mL), saturated sodium bicarbonate (2×50 mL), and with brine (50 mL). The organic layer was separated, dried (sodium sulfate), filtered, and then the filtrate was evaporated to afford a solid, which was purified by trituration in 50% ethyl acetate in hexane (100 mL). The mixture was filtered, and then dried to give 1.46 g of additional desired product for a combined weight of 3.76 g (99%): H¹NMR (400 MHz DMSO-d₆) δ 9.31, 9.18, 7.51, 7.21-6.92, 6.71, 5.12, 4.35, 1.48; MS(APCI⁺) m/z 460.

Step B

### (3R,5R)-7-{3,4-bis-(4-fluoro-phenyl)-1-isopropyl-5-[(pyridin-2-yl Methyl)-carbamoyl]-1H-pyrrol-2-yl}-3,5-dihydroxy-heptanoate Sodium Salt

The title compound was prepared from 3,4-Bis-(4-fluoro-phenyl)-5-formyl-1-isopropyl-1H-pyrrole-2-carboxylic acid (pyridine-2-ylmethyl)-amide by the methods described in Steps B, C (Method B), and D-F of Example 67: m.p: 200-205° C.; H¹ NMR (400 MHz DMSO-d₆) δ 8.42, 8.34, 7.13, 7.05-6.83, 6.65, 4.76, 4.53, 4.25, 3.66, 3.53, 2.71-2.57, 2.51-2.36, 1.93, 1.78-1.68, 1.60-1.13; MS(APCI⁺) m/z 590.

### Example 70

### trans-(3R,5S)-7-{3,4-bis-(4-fluoro-phenyl)-1-isopropyl-5-[(pyridin-2-ylmethyl)-carbamoyl]-1H-pyrrol-2-yl)-3,5-dihydroxy-hept-6-enoate Sodium Salt

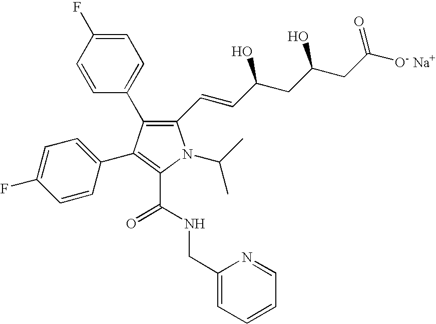

The title compound was prepared from trans-(3R,5S)-7-{3,4-bis-(4-fluoro-phenyl)-1-isopropyl-5-[(pyridin-2-ylmethyl)-carbamoyl]-1H-pyrrol-2-yl}-3,5-dihydroxy-hept-6-enoic acid methyl ester by the procedure described in Step C of Example 2: H¹ NMR (400 MHz DMSO-d₆) δ 8.49, 8.35, 7.48, 7.41, 7.14, 7.03-6.83, 6.66, 6.41, 5.31, 4.96, 4.63, 4.29, 4.06, 3.49, 1.95-1.64, 1.46, 1.39-1.00; MS(APCI⁺) m/z 590.

### Example 71

### (3R,5R)-7-[5-(3-dimethylsulfamoyl-benzylcarbamoyl)-3,4-bis-(4-fluoro-phenyl)-1-isopropyl-1H-pyrrol-2-yl]-3,5-dihydroxy-heptanoate Sodium Salt

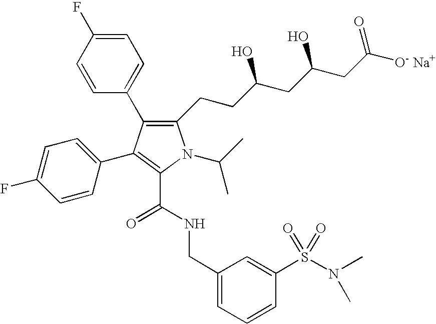

The title compound was prepared from 3,4-bis-(4-fluoro-phenyl)-5-formyl-1-isopropyl-1H-pyrrole-2-carboxylic acid (prepared in Step G of Example 1) by the methods described in Step A of Example 69 and Steps B, C (Method B), and D-F of Example 67, substituting 3-aminomethyl-N,N-dimethyl-benzenesulfonamide hydrochloride (L. F. McBurney et. al, JACS, 62: 2099 1940) for 3-amino-N,N-dimethyl-benzamide in Step A: m.p: 172-175° C.; H¹ NMR (400 MHz DMSO-d₆) δ 8.55, 7.70, 7.56, 7.44, 7.27, 7.09, 7.27, 7.09-6.81, 4.78, 4.51, 4.26, 3.68, 3.55, 2.73-2.51, 1.99-1.91, 1.79-1.69, 1.62-1.17; MS(APCI⁻) m/z 696.

### Example 72

### trans-(3R,5S)-7-[5-(3-dimethylsulfamoyl-benzylcarbamoyl)-3,4-bis-(4-fluoro-phenyl)-1-isopropyl-1H-pyrrol-2-yl]-3,5-dihydroxy-hept-6-enoate Sodium Salt

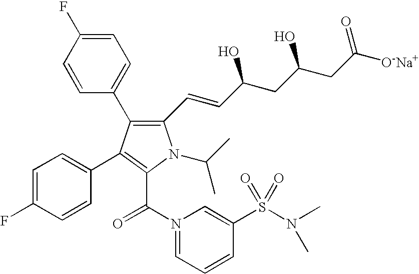

The title compound was prepared from trans-(3R,5S)-7-[5-(3-dimethylsulfamoyl-benzylcarbamoyl)-3,4-bis-(4-fluoro-phenyl)-1-isopropyl-1H-pyrrol-2-yl]-3,5-dihydroxy-hept-6-enoic acid methyl ester by the procedure described in Step C of Example 2: H¹ NMR (400 MHz DMSO-d₆) δ 8.59, 7.47-7.59, 7.42, 7.22, 7.01-6.80, 6.38, 5.30, 4.96, 4.55, 4.27, 4.05, 3.48, 2.51, 1.93-1.84, 1.73-1.63,1,48-1.26, 1.09-0.97; MS(APCI⁻) m/z 695.

### Example 73

### (3R,5R)-7-[3,4-bis-(4-fluoro-phenyl)-1-isopropyl-5-(3-methoxycarbonyl-phenylcarbamoyl)-1H-pyrrol-2-yl]-3,5-dihydroxy-heptanoate Sodium Salt

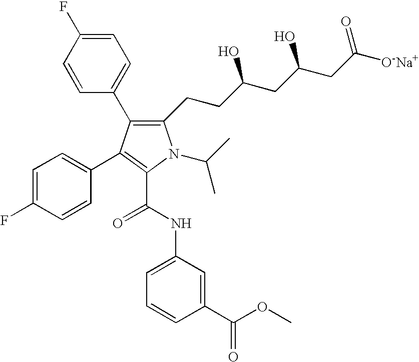

The title compound was prepared from 3,4-bis-(4-fluoro-phenyl)-5-formyl-1-isopropyl-1H-pyrrole-2-carboxylic acid (prepared in Step G of Example 1) by the methods described in Steps A-B, C (Method B), and D-F of Example 67, substituting methyl 3-aminobenzoate for 3-amino-N,N-dimethyl-benzamide in Step A and methanol for ethanol:water in Step F: H¹ NMR (400 MHz DMSO-d₆) δ 10.22, 7.67, 7.59-7.46, 7.32, 7.09-6.82, 4.79, 4.58, 3.78, 3.67, 3.55, 2.74-2.37, 1.93, 1.78-1.67, 1.63-1.15; MS(APCI⁻) m/z 635.

### Example 74

### trans-(3R,5S)-7-[3,4-bis-4-fluoro-phenyl)-1-isopropyl-5-(3-methoxycarbonyl-phenylcarbamoyl)-1H-pyrrol-2-yl}-3,5-dihydroxy-hept-6-enoate Sodium Salt

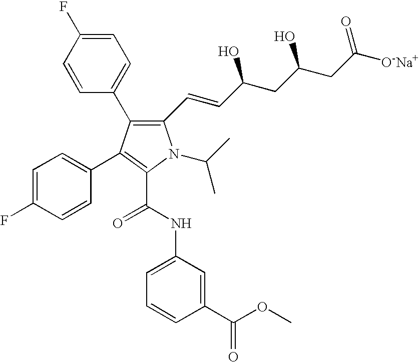

The title compound was prepared from trans-(3R,5S)-7-[3,4-bis-4-fluoro-phenyl)-1-isopropyl-5-(3-methoxycarbonyl-phenylcarbamoyl)-1H-pyrrol-2-yl)-3,5-dihydroxy-hept-6-enoic acid methyl ester by the procedure described in Step C of Example 2: H¹ NMR (400 MHz DMSO-d₆) δ 10.24, 8.17, 7.60-7.41, 7.34, 7.06-6.86, 6.43, 5.37, 4.99, 4.66, 4.08, 3.79, 3.50, 1.91, 1.75-1.64, 1.49, 1.40-1.30, 1.11-1.02; MS(APCI⁻) m/z 615.

### Example 75

### (3R,5R)-7-[3,4-bis-(4-fluoro-phenyl)-1-isopropyl-5-(3-oxycarbonyl-phenylcarbamoyl)-1H-pyrrol-2-yl]-3,5-dihydroxy-heptanoate Disodium Salt

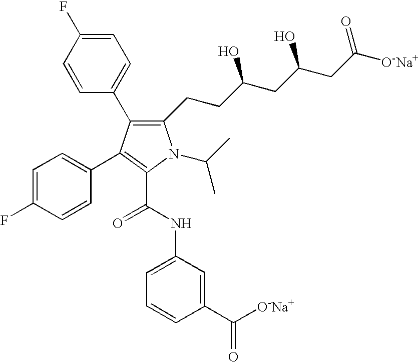

To a solution of (3R,5R)-7-[3,4-bis-(4-fluoro-phenyl)-1-isopropyl-5-(3-methoxycarbonyl-phenylcarbamoyl)-1H-pyrrol-2-yl]-3,5-dihydroxy-heptanoic acid methyl ester [prepared by the methods described in Steps A-B, C (Method B), and D-E of Example 67, substituting methyl 3-aminobenzoate for 3-amino-N,N-dimethyl-benzamide in Step A] (0.33 g, 0.51 mmoles) methanol (6 mL) was added 1.028 N aqueous sodium hydroxide (1.8 mL, 1.6 mmoles). The reaction mixture was stirred at reflux for 3 hrs and then evaporated to give a yellow oil, which was suspended in 100 mL of 90% dichloromethane in methanol and then filtered. The filtrate was evaporated to provide a solid, which was suspended in 50 mL of water and then acidified with 1 N HCl to pH=2 to form a precipitate. The mixture was filtered to provide a solid and then the material was dissolved in methanol (6 mL) and treated with 1.028 N aqueous sodium hydroxide (0.73 mL, 0.75 mmoles). The reaction mixture was stirred at room temperature for 18 hrs and then evaporated to give a solid, which was purified by trituration in anhydrous diethyl ether (30 mL) at to give 296 mg (88%) of the desired product as a white solid.

H¹ NMR (400 MHz DMSO-d₆) δ 9.90, 7.62-7.42, 7.28, 7.10-6.82, 4.76, 4.56, 3.68, 3.55, 2.74-2.37, 1.95, 1.81-1.69, 1.63-1.15; MS(APCI⁻) m/z 621.

### Example 76

### (3R,5R)-7-{3,4-bis-(4-fluoro-phenyl)-1-isopropyl-5-[(pyridin-3-ylmethyl)-carbamoyl]-1H-pyrrol-2-yl)-3,5-dihydroxy-hept-6-enoate Sodium Salt

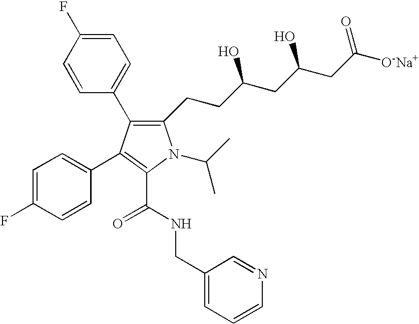

The title compound was prepared from 3,4-bis-(4-fluoro-phenyl)-5-formyl-1-isopropyl-1H-pyrrole-2-carboxylic acid (prepared in Step G of Example 1) by the methods described in Steps A-B, C (Method B), and D-F of Example 67, substituting 3-(aminomethyl)-pyridine for 3-amino-N,N-dimethyl-benzamide in Step A and methanol for ethanol:water in Step F: H¹ NMR (400 MHz DMSO-d₆) δ 8.47, 8.34, 8.32, 7.65, 7.23, 7.13, 7.05-6.75, 4.76,4.48, 4.16, 3.66, 3.53, 2.69-2.33, 1.92, 1.77-1.65, 1.61-1.13; MS(APCI⁻) m/z 592.

### Example 77

### trans-(3R,5S)-7-{3,4-bis-(4-fluoro-phenyl)-1-isopropyl-5-[(pyridin-3-ylmethyl)-carbamoyl]-1H-pyrrol-2-yl}-3,5-dihydroxy-hept-6-enoate Sodium Salt

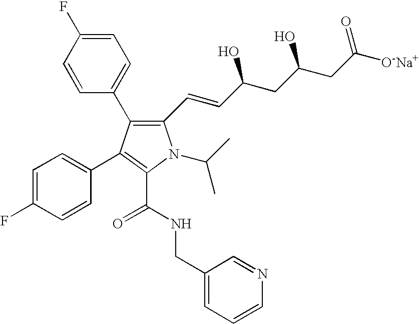

The title compound was prepared from trans-(3R,5S)-7-{3,4-bis-(4-fluoro-phenyl)-1-isopropyl-5-[(pyridin-3-ylmethyl)-carbamoyl]-1H-pyrrol-2-yl}-3,5-dihydroxy-hept-6-enoic acid methyl ester by the procedure described in Step C of Example 2, substituting methanol for ethanol:water: H¹ NMR (400 MHz DMSO-d₆) δ 8.53, 8.34, 8.23, 7.44, 7.25-7.11, 7.08-6.78, 6.38, 5.31, 4.96, 4.56, 4.19, 4.05, 3.48, 1.90, 1.75-1.63, 1.49-1.27, 1.11-0.98; MS(APCI⁻) m/z 590.

### Example 78

### (3R,5R)-7-{3-(4-fluoro-phenyl)-1-isopropyl-5-[(5-methyl-isoxazole-3-ylmethyl)-carbamoyl]-4-phenyl-1H-pyrrol-2-yl)3,5-dihydroxy-heptanoate Sodium Salt

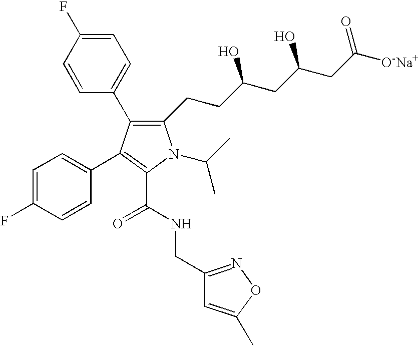 Step A

### (3R,5R)-7-{3-(4-fluoro-phenyl)-1-isopropyl-5-[(5-methyl-isoxazole-3-ylmethyl)-carbamoyl]-4-phenyl-1H-pyrrol-2-yl}-3,5-dihydroxy-heptanoic Acid Methyl Ester and Z-(3R,5R)-7-[5-(2-amino-4-oxo-pent-2-enylcarbamoyl)-3-(4-fluoro-phenyl)-1-isopropyl-4-phenyl-1H-pyrrol-2-yl]-3,5-dihydroxy-heptanoic Acid Methyl Ester

To a solution of cis/trans-(3R,5S)-7-{3-(4-fluoro-phenyl)-1-isopropyl-5-[(5-methyl-isoxazole-3-ylmethyl)-carbamoyl]-4-phenyl-1H-pyrrol-2-yl}-3,5-dihydroxy-hept-6-enoic acid methyl ester prepared from 4-(4-fluoro-phenyl)-5-formyl-1-isopropyl-3-phenyl-1H-pyrrole-2-carboxylic acid by the procedure described in Steps A, B, C (Method B), and D and E of Example 67, substituting C-(5-methyl-isoxazol-3-yl)-methylamine for 3-amino-N,N-dimethylbenzamide in Step A and methanol for ethanol:water in Step B (0.64 g, 1.1 mmoles) in methanol (50 mL) was added 10% Palladium on carbon (0.125 g, 0.12 mmoles Pd) and then the reaction mixture was violently shaken under hydrogen (5 psi) for 1.5 hrs. The reaction mixture was filtered to remove the catalyst and then the filtrate was evaporated to give a colorless oil, which was purified by flash chromatography (silica gel, 95% ethyl acetate in methanol) to afford 219 mg (34% chr) of (3R,5R)-7-{3-(4-fluoro-phenyl)-1-isopropyl-5-[(5-methyl-isoxazole-3-ylmethyl)-carbamoyl]-4-phenyl-1H-pyrrol-2-yl}-3,5-dihydroxy-heptanoic acid methyl ester and 177 mg (27% chr) of, Z-(3R,5R)-7-[5-(2-amino-4-oxo-pent-2-enylcarbamoyl)-3-(4-fluoro-phenyl)-1-isopropyl-4-phenyl-1H-pyrrol-2-yl]-3,5-dihydroxy-heptanoic acid methyl ester as as white solids

Step B

(3R,5R)-7-{3-(4-fluoro-phenyl)-1-isopropyl-5-[(5-methyl-isoxazole-3-ylmethyl)-carbamoyl]-4-phenyl-1H-pyrrol-2-yl)-3,5-dihydroxy-heptanoate Sodium Salt

The title compound was prepared from of (3R,5R)-7-{3-(4-fluoro-phenyl)-1-isopropyl-5-[(5-methyl-isoxazole-3-ylmethyl)-carbamoyl]-4-phenyl-1H-pyrrol-2-yl}-3,5-dihydroxy-heptanoic acid methyl ester (prepared in Step A of Example 67 by the method described in Step C of Example 2, substituting methanol for ethanol:water in Step F: m.p: 193-195° C.; H¹ NMR (400 MHz DMSO-d₆) δ 8.39, 7.62, 7.07-6.85, 5.42, 4.76, 4.50, 4.11, 3.65, 3.53, 2.70-2.34, 2.24, 1.93, 1.77-1.67, 1.59-1.27, 1.24-1.14; MS(APCI⁻) m/z 578.

### Example 79

### trans-(3R,5 S)-7-{3-(4-fluoro-phenyl)-1-isopropyl-5-[(5-methyl-isoxazole-3-yl Methyl)-carbamoyl]-4-phenyl-1H-pyrrol-2-yl}-3,5-dihydroxy-hept-6-enoate Sodium Salt

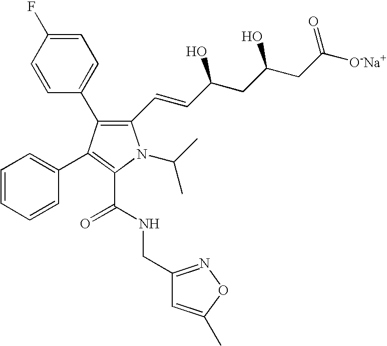

The title compound was prepared from trans-(3R,5S)-7-{3-(4-fluoro-phenyl)-1-isopropyl-5-[(5-methyl-isoxazole-3-ylmethyl)-carbamoyl]-4-phenyl-1H-pyrrol-2-yl}-3,5-dihydroxy-hept-6-enoic acid methyl ester by the procedure described in Step C of Example 2, substituting methanol for ethanol:water: m.p: 200-203° C.; H¹ NMR (400 MHz DMSO-d₆) δ 8.47, 7.39, 7.10-6.87, 6.40, 5.42, 5.30, 4.95, 4.58, 4.15, 4.05, 3.49, 2.24, 1.91, 1.75-1.65, 1.44, 1.39-1.28, 1.10-0.99; MS(APCI⁻) m/z 576.

### Example 80

### Z-(3R,5R)-7-[5-(2-amino-4-oxo-pent-2-enylcarbamoyl)-3-(4-fluoro-phenyl)-1-isopropyl-4-phenyl-1H-pyrrol-2-yl]-3,5-dihydroxy-heptanoate Sodium Salt

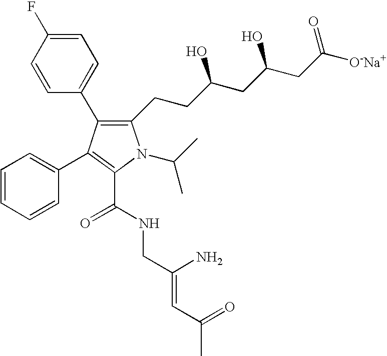

The title compound was prepared from Z-(3R,5R)-7-[5-(2-amino-4-oxo-pent-2-enylcarbamoyl)-3-(4-fluoro-phenyl)-1-isopropyl-4-phenyl-1H-pyrrol-2-yl]-3,5-dihydroxy-heptanoic acid methyl ester (prepared in Step A of Example 78 by the procedure described in Step C of Example 2: H¹ NMR (400 MHz DMSO-d₆) δ 9.14, 8.28, 7.52, 7.15-6.86, 4.75, 4.66, 4.51, 3.71-3.57, 3.52, 2.70-2.57, 2.52-2.36, 1.92, 1.79-1.67, 1.59-1.12; MS(APCI⁻) m/z 580.

### Example 81

### (3R,5R)-7-[ ]-ethyl-3-(4-fluoro-phenyl)-5-(4-methoxy-benzylcarbamoyl)-4-methyl-1H-pyrrol-2-yl}-3,5-dihydroxy-heptanoate Sodium Salt

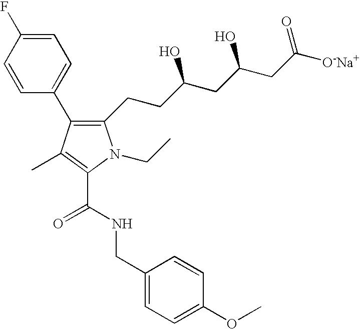

The title compound was prepared from 1-ethyl-4-(4-fluoro-phenyl)-5-formyl-3-methyl-1H-pyrrole-2-carboxylic acid (prepared by the method described in Step F of Example 22) by the methods described in Steps G-L of Example 21 substituting 4-methoxy-benzylamine for aniline, 70% HF:pyridine for 48% aqueous HF in Step I, and methanol for ethanol:water in Step L: H¹ NMR (400 MHz DMSO-d₆) δ 8.22, 7.57, 7.24-7.10, 6.83, 4.70, 4.31, 4.06, 3.70-3.58, 3.53-3.42, 2.64-2.32, 1.98-1.84, 1.75-1.64, 1.51-0.99; MS(APCI⁻) m/z 527.

### Example 82

### trans-(3R,5S)-7-{3-(4-fluoro-phenyl)-1-isopropyl-5-[(5-methyl-isoxazole-3-yl Methyl)-carbamoyl]-4-phenyl-1H-pyrrol-2-yl)-3,5-dihydroxy-hept-6-enoate Sodium Salt

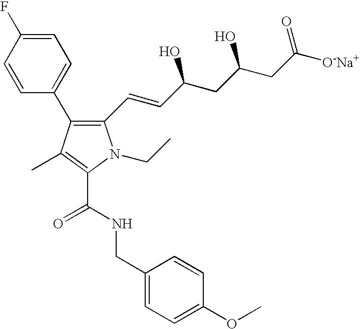

The title compound was prepared from trans-(3R,5S)-7-{3-(4-fluoro-phenyl)-1-isopropyl-5-[(5-methyl-isoxazole-3-ylmethyl)-carbamoyl]-4-phenyl-1H-pyrrol-2-yl}-3,5-dihydroxy-heptanoic acid methyl ester (prepared by the method described in Step J of Example 21) by the method described in Step L of Example 21 substituting methanol for ethanol:water: H¹ NMR (400 MHz DMSO-d₆) δ 8.35, 7.37, 7.25-7.08, 6.83, 6.27, 5.39, 4.90, 4.32, 4.12-4.00, 3.67, 3.52, 1.97-1.83, 1.77-1.64, 1.41-1.29, 1.19-1.03; MS(APCI⁻) m/z 525.

### Example 83

### (3R,5R)-7-{1-ethyl-3-(4-fluoro-phenyl)-4-methyl-5-[(5-methyl-pyrazin-2-ylmethyl)-carbamoyl]-1H-pyrrol-2-yl 1-3,5-dihydroxy-heptanoate Sodium Salt

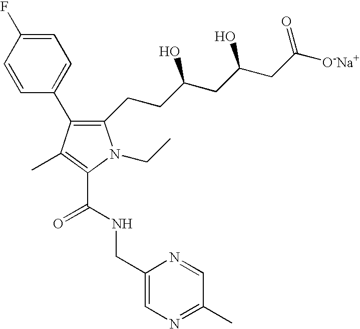

The title compound was prepared from 1-ethyl-4-(4-fluoro-phenyl)-5-formyl-3-methyl-1H-pyrrole-2-carboxylic acid (prepared by the method described in Step F of Example 22) by the methods described in Steps G-L of Example 21 substituting 2-(aminomethyl)-5-methylpyrazine for aniline, 70% HF:pyridine for 48% aqueous HF in Step I, and methanol for ethanol:water in Step L: H¹ NMR (400 MHz DMSO-d₆) δ 8.43, 8.34, 7.60, 7.15, 4.71, 4.48, 4.06, 3.63, 3.48, 2.65-2.31, 2.05-1.84, 1.76-1.64, 1.53-1.04; MS(APCI⁻) m/z 513.

### Example 84

### cis/trans-(3R,5 S)-7-{1-ethyl-3-(4-fluoro-phenyl)-4-methyl-5-[(5-methyl-pyrazin-2-ylmethyl)-carbamoyl]-1H-pyrrol-2-yl}-3,5-dihydroxy-hept-6-enoate Sodium Salt

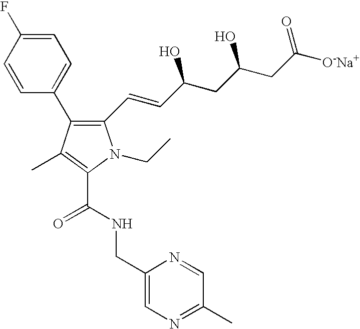

The title compound was prepared from cis/trans-(3R,5S)-7-{1-ethyl-3-(4-fluoro-phenyl) -4-methyl-5-[(5-methyl-pyrazin-2-ylmethyl)-carbamoyl]-1H-pyrrol-2-yl-3,5-dihydroxy-heptanoic acid methyl ester (prepared by the method described in Step J of Example 21) by the method described in Step L of Example 21 substituting methanol for ethanol:water: H¹ NMR (400 MHz DMSO-d₆) δ 8.50-8.37, 7.31, 7.20-7.04, 6.28, 6.16, 5.52, 5.40, 4.91, 4.49, 4.07, 3.53, 2.00-1.87, 1.78-1.66, 1.54-1.30, 1.20-0.98; MS(APCI⁻) m/z 511.

### Example 85

### (3R,5R)-7-[5-(4-dimethylcarbamoyl-phenylcarbamoyl)-3,4-bis-(4-fluoro-phenyl)-1-isopropyl-1H-pyrrol-yl]-3,5-dihydroxy-heptanoate Sodium Salt

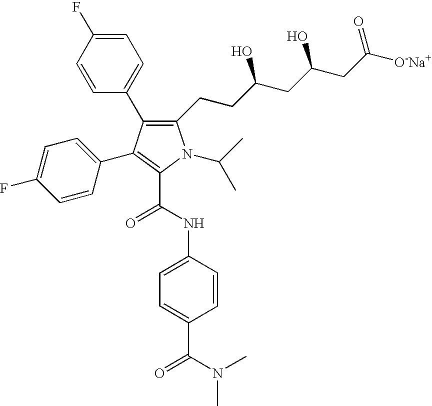

The title compound was prepared from 3,4-bis-(4-fluoro-phenyl)-5-formyl-1-isopropyl-1H-pyrrole-2-carboxylic acid (prepared in Step G of Example 1) by the methods described in Steps A, B, C (Method A), and D-F of Example 67, substituting 4-aminomethyl-N,N-dimethyl-benzamide (H. Wenker, JACS, 60: 1080 1938) for 3-amino-N,N-dimethyl-benzamide in Step A: m.p: 220-223° C.; H¹ NMR (400 MHz DMSO-d₆) δ 10.18, 7.63, 7.44, 7.24, 7.09-6.82, 4.78, 4.58, 3.66, 3.54, 2.87, 2.72-2.37, 1.99-1.68, 1.64-1.12; MS(APCI⁻) m/z 648.

### Example 86

### trans-(3R,5S)-7-[5-(4-dimethylcarbamoyl-phenylcarbamoyl)-3,4-bis-(4-fluoro-phenyl)-1-isopropyl-1H-pyrrol-yl]-3,5-dihydroxy-hept-6-enoate Sodium Salt

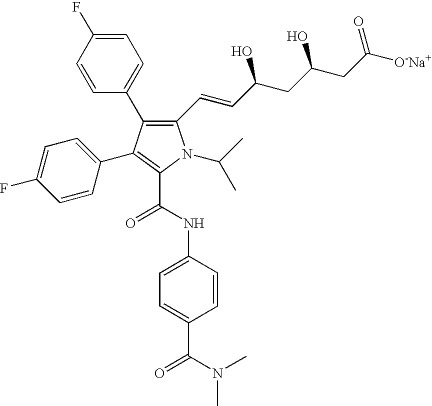

The title compound was prepared from trans-(3R,5S)-7-[5-(4-dimethylcarbamoyl-phenylcarbamoyl)-3,4-bis-(4-fluoro-phenyl)-1-isopropyl-1H-pyrrol-yl]-3,5-dihydroxy-hept-6-enoic acid methyl ester (prepared in an analogous fashion to Step D of Example 67) by the procedure described in Step C of Example 2: m.p: 222-2250C; H¹ NMR (400 MHz DMSO-d₆) δ 10.21, 7.26, 7.05-6.86, 6.43, 5.37, 4.65, 4.08, 3.49, 2.87, 1.91, 1.70, 1.49, 1.35, 1.06; MS(APCI⁺) m/z 628.

### Example 87

### (3R,5R)-7-[5-(4-dimethylsulfamoyl-benzylcarbamoyl)-3,4-bis-(4-fluoro-phenyl)-1-isopropyl-1H-pyrrol-2-yl]-3,5-dihydroxy-heptanoate Sodium Salt

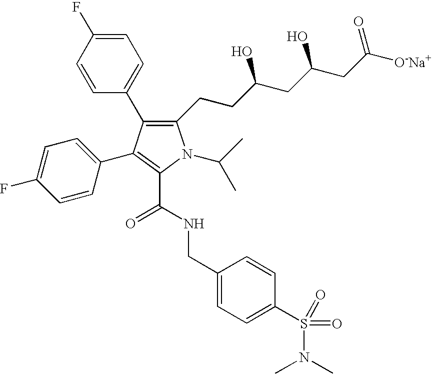

The title compound was prepared from 3,4-bis-(4-fluoro-phenyl)-5-formyl-1-isopropyl-1H-pyrrole-2-carboxylic acid by the methods described in Step A of Example 69 and Steps B, C (Method A), and D-F of Example 67, substituting 4-aminomethyl-N,N-dimethyl-benzenesulfonamide hydrochloride (L. F. McBurney et. al, JACS, 62: 2099 1940) for 3-amino-N,N-dimethyl-benzamide in Step A: H¹ NMR (400 MHz DMSO-d₆) δ 8.55, 7.65, 7.47,7.10, 7.05-6.84,4.76,4.50, 4.26, 3.66, 3.53, 2.62, 2.56-2.36, 1.92, 1.72, 1.63-1.10; MS(APCI⁻) m/z 698.

### Example 88

### trans-(3R,5 S)-7-[5-(4-dimethylsulfamoyl-benzylcarbamoyl)-3,4-bis-(4-fluoro-phenyl)-1-isopropyl-1H-pyrrol-2-yl]-3,5-dihydroxy-hept-6-enoate Sodium Salt

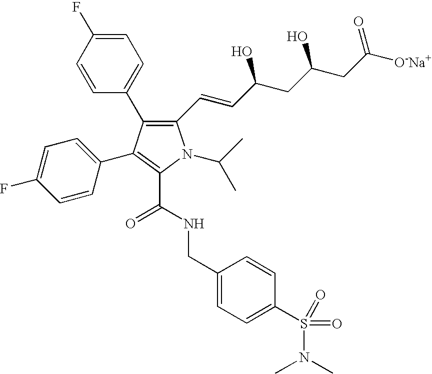

The title compound was prepared from trans-(3R,5S)-7-[5-(4-dimethylsulfamoyl-benzylcarbamoyl)-3,4-bis-(4-fluoro-phenyl)-1-isopropyl-1H-pyrrol-2-yl]-3,5-dihydroxy-hept-6-enoic acid methyl ester by the procedure described in Step C of Example 2: H¹ NMR (400 MHz DMSO-d₆) δ 8.55, 7.65, 7.47, 7.09, 7.05-6.83, 4.76, 4.49,4.26, 3.66, 3.53, 2.63, 2.56-2.33, 1.92, 1.72, 1.61-1.12; MS(APCI⁻) m/z 678.

### Example 89

### (3R,5R)-7-[3-(4-Fluorophenyl)-1-isopropyl-5-(2-pyridin-3-ylethylcarbamoyl)-4-phenyl-1H-pyrrol-2-yl]-3,5-dihydroxyheptanoic Acid Sodium Salt

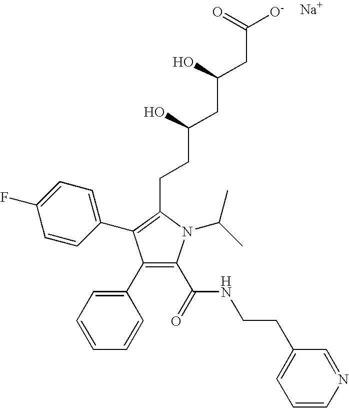 Step A

### 4-(4-Fluorophenyl)-5-formyl-1-isopropyl-3-phenyl-1H-pyrrole-2-carboxylic Acid (2-pyridin-3-ylethyl)amide

To a stirred mixture of 4-(4-fluorophenyl)-5-formyl-1-isopropyl-3-phenyl-1H-pyrrole-2-carboxylic acid (Example 1, Step G, 2.80 g, 7.97 mmol) in dry dichloromethane (80 mL) under a nitrogen atmosphere was added dry DMF (15 μL, 0.199 mmol) followed by oxalyl chloride (0.834 mL, 9.56 mmol) dropwise. Gas evolution occurred soon after the addition was complete. The mixture was stirred at room temperature overnight and was then concentrated in vacuo to give a quantitative yield of the acid chloride intermediate which was used without further purification. A solution of this acid chloride in ethyl acetate (20 mL) was added portionwise to a vigorously stirred mixture of 3-(2-aminoethyl)pyridine (0.974 g, 7.97 mmol) and sodium carbonate (1.27 g, 12.0 mmol) in ethyl acetate (32 mL) and water (8 mL) at 0-5° C. The resulting mixture was stirred at 0-5° C. for 1 hr and at room temperature overnight and was then partitioned between ethyl acetate (100 mL) and water (100 mL). The organic phase was separated, washed with water (50 mL) and brine (50 mL), dried over anhydrous Na₂SO₄ and concentrated in vacuo, and the residue was purified by silica gel chromatography (1-2% methanol in dichloromethane or 75-85% ethyl acetate in hexanes) to give 1.53 g (42%) of the title compound as a yellow solid: mp 192-194° C.; MS(APCI⁺) m/z 456.

Step B

### 4-(4-Fluorophenyl)-5-hydroxymethyl-1-isopropyl-3-phenyl-1H-pyrrole-2-carboxylic Acid (2-pyridin-3-ylethyl)amide

A stirred solution of 4-(4-fluorophenyl)-5-formyl-1-isopropyl-3-phenyl-1H-pyrrole-2-carboxylic acid (2-pyridin-3-ylethyl)amide from Step A (1.45 g, 3.18 mmol) in dry THF (32 mL) under a nitrogen atmosphere was cooled in an ice-salt bath and treated with lithium tri-tert-butoxyaluminohydride (1M in THF, 3.98 mL) dropwise over 2 mins. The resulting mixture was stirred at −5-0° C. for 1.5 hrs and was then quenched slowly with saturated aq. NH₄Cl (12 mL). The resulting heterogeneous mixture was diluted with 1M HCl (12 mL), water (30 mL) and ethyl acetate (30 mL) and stirred for ˜10 mins to allow the solids to dissolve, and then the layers were separated. The aqueous phase was extracted with ethyl acetate (60 mL), and the combined organic phase was washed with saturated aq. NaHCO₃ (20 mL) and brine (20 mL), dried over anh. MgSO₄ and concentrated in vacuo. The residue was purified by silica gel chromatography (1-5% methanol in dichloromethane) to give 1.47 g (99%) of the title compound as a yellow solid: mp 167-169° C.; MS(APCI⁻) m/z 456.

Step C

### [3-(4-Fluorophenyl)-1-isopropyl-4-phenyl-5-(2-pyridin-3-ylethylcarbamoyl)-1H-pyrrol-2-ylmethyl]triphenylphosphonium Bromide

To a stirred slurry of 4-(4-fluorophenyl)-5-hydroxymethyl-1-isopropyl-3-phenyl-1H-pyrrole-2-carboxylic acid (2-pyridin-3-ylethyl) amide from Step B (1.40 g, 3.06 mmol) in dry acetonitrile (62 mL) under nitrogen was added triphenylphosphine hydrobromide (1.10 g, 3.21 mmol). The resulting homogeneous mixture was placed in a 65° C. heating bath and stirred at this temperature for 4 hrs. The heating bath was removed, and the mixture was stirred at room temperature overnight and then concentrated in vacuo to give 2.32 g (94%) of the title compound as a light yellow amorphous solid: MS(APCI⁺) m/z 702.

Step D

### ((4R,6S)-6-{2-[3-(4-Fluorophenyl)-1-isopropyl-4-phenyl-5-(2-pyridin-3-ylethylcarbamoyl)-1H-pyrrol-2-yl]vinyl}-2,2-dimethyl-[1,3]dioxan-4-yl)acetic Acid Tert-Butyl Ester

A solution of [3-(4-fluorophenyl)-1-isopropyl-4-phenyl-5-(2-pyridin-3-ylethylcarbamoyl)-1H-pyrrol-2-ylmethyl]triphenylphosphonium bromide from step C (2.25 g, 2.87 mmol) in dry DMSO (20 mL) and THF (100 mL) under a nitrogen atmosphere was cooled to −78° C., affording a pale yellow slurry, and treated with NaHMDS (1M in THF, 3.45 mL) dropwise over ˜2 min with vigorous stirring. The resulting orange slurry was stirred at −78° C. for 5-6 mins and was then treated with a solution of ((4R,6S)-6-formyl-2,2-dimethyl-[1,3]dioxan-4-yl)acetic acid tert-butyl ester (1.75 g, 6.77 mmol, Syn. Comm. 2003, 33(13), 2275-83) in dry THF (15 mL) dropwise over 3 mins. The mixture was stirred at −78° C. for 40 mins, the cooling bath was removed and the mixture was allowed to warm to room temperature and stir for 1 hr. The mixture was then quenched slowly with saturated aq. NH₄Cl (20 mL) and partitioned between water (100 mL) and ethyl acetate (100 mL). The organic phase was separated, washed with water (50 mL) and brine (50 mL), dried over anhydrous MgSO₄ and concentrated in vacuo, and the residue was purified by silica gel chromatography (40-80% ethyl acetate in hexanes) to give 2.30 g of a pale yellow foam which consisted of ˜70% product by weight (˜1.60 g, ˜80% yield; ˜1: I mixture of cis/trans alkene isomers) and residual Ph₃PO. The product mixture was used as is in the next step. MS(APCI⁺) m/z 682.

Step E

### ((4R,6R)-6-{2-[3-(4-Fluorophenyl)-1-isopropyl-4-phenyl-5-(2-pyridin-3-ylethylcarbamoyl)-1H-pyrrol-2-yl]ethyl}-2,2-dimethyl-[1,3]dioxan-4-yl)acetic Acid Tert-Butyl Ester

A solution of ((4R,6S)-6-{2-[3-(4-fluorophenyl)-1-isopropyl-4-phenyl-5-(2-pyridin-3-ylethylcarbamoyl)-1H-pyrrol-2-yl]vinyl}-2,2-dimethyl-[1,3]dioxan-4-yl)acetic acid tert-butyl ester from Step D (11.14 g, 70% pure, 1.17 mmol) in methanol (50 mL) was treated with 10% palladium-on-carbon (0.25 g), and the mixture was shaken on a Parr apparatus under a hydrogen atmosphere (50 psi) for 7 hrs. The mixture was then filtered to remove the catalyst, the filtrate was concentrated in vacuo, and the residue was purified by silica gel chromatography (50-95% ethyl acetate/hexanes) to give 980 mg of a white foam which consisted of ˜65% product by weight (˜0.64 g, ˜80% yield) and residual Ph₃PO from the starting mixture. The product mixture was used as is in the next step. MS(APCI⁺) m/z 684.

Step F

### (3R,5R)-7-[3-(4-Fluorophenyl)-1-isopropyl-4-phenyl-5-(2-pyridin-3-ylethylcarbamoyl)-1H-pyrrol-2-yl]-3,5-dihydroxyheptanoic Acid Tert-Butyl Ester

A solution of ((4R,6R)-6-{2-[3-(4-fluorophenyl)-1-isopropyl-4-phenyl-5-(2-pyridin-3-ylethylcarbamoyl)-1H-pyrrol-2-yl]ethyl}-2,2-dimethyl-[1,3]dioxan-4-yl)acetic acid tert-butyl ester from Step E (0.98 g, ˜65% pure, 0.93 mmol) in methanol (28 mL) was treated with 1N aq. HCl (2.33 mL), and the mixture was stirred at room temperature for 4 hrs. The solvent was then removed in vacuo, and the residue was diluted carefully with saturated aq. NaHCO₃ (10 mL) and water (20 mL) and extracted with ethyl acetate (30 mL). The organic phase was washed with brine (10 mL), dried over anhydrous Na₂SO₄ and concentrated in vacuo, and the residue was purified by silica gel chromatography (1-4% methanol in dichloromethane) to give 505 mg (84%) of the title compound as a white solid: mp 155-1560C; MS(APCI⁺) m/z 644.

Step G

### (3R,5R)-7-[3-(4-Fluorophenyl)-1-isopropyl-5-(2-pyridin-3-ylethylcarbamoyl)-4-phenyl-1H-pyrrol-2-yl]-3,5-dihydroxyheptanoic Acid Sodium Salt

A solution of (3R,5R)-7-[3-(4-fluorophenyl)-1-isopropyl-4-phenyl-5-(2-pyridin-3-ylethylcarbamoyl)-1H-pyrrol-2-yl]-3,5-dihydroxyheptanoic acid tert-butyl ester from Step F (0.325 g, 0.505 mmol) in methanol (11 mL) was treated with 1N aq. NaOH (0.516 mL), and the reaction mixture was stirred at room temperature for 3 days. The solvent was then removed in vacuo, and the residue was taken up in a minimum of 10% methanol in dichloromethane and filtered to remove any residual NaOH. The filtrate was concentrated in vacuo, the residue was triturated with diethyl ether (˜25 mL) and the solid was collected by filtration and dried in vacuo to give 234 mg (76%) of the title compound as a white solid: NMR (400 MHz, DMSO-d₆) δ 8.31, 8.23, 7.99, 7.52, 7.37, 7.20-6.90, 4.74, 4.42, 3.66, 3.52, 3.20, 2.60, 2.50, 2.40,1.92, 1.75, 1.50,1.40, 1.34, 1.20; MS(APCI⁻) m/z 586.

### Example 90

### (3R,5R)-7-[3-(4-Fluorophenyl)-1-isopropyl-5-(2-pyridin-2-ylethylcarbamoyl)-4-phenyl-1H-pyrrol-2-yl]-3,5-dihydroxyheptanoic Acid Sodium Salt

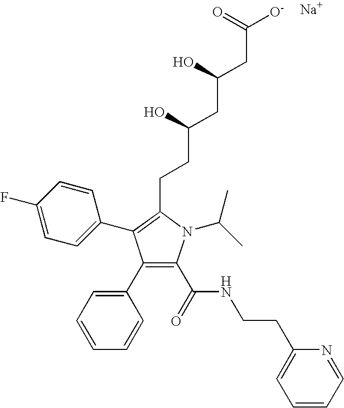

The title compound was prepared by a method analogous to that described for the preparation of Example 89, substituting 2:(2-aminoethyl)pyridine for 3-(2-aminoethyl)pyridine in Step A. NMR (400 MHz, DMSO-d₆) δ 8.37,7.91, 7.66, 7.56, 7.13-6.90, 4.75, 4.45, 3.66, 3.52, 3.30, 2.60, 2.43, 1.92, 1.71, 1.52, 1.42, 1.33, 1.19; HRMS (ESI⁺) found 588.2855.

### Example 91

### (3R,5R)-7-[3-(4-Fluorophenyl)-1-isopropyl-5-(phenethylcarbamoyl)-4-phenyl-1H-pyrrol-2-yl]-3,5-dihydroxyheptanoic Acid Sodium Salt

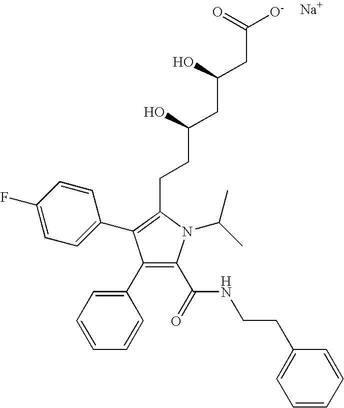

The title compound was prepared by a method analogous to that described for the preparation of Example 89, substituting phenethylamine for 3-(2-aminoethyl)pyridine in Step A. MS(APCI⁻) m/z 585; mp 197-200° C.

### Example 92

### 3R,5R)-7-[3-(4-Fluorophenyl)-1-isopropyl-5-[(1H-benzimidazol-2-ylmethyl)carbamoyl]-4-phenyl-1H-pyrrol-2-yl]-3,5-dihydroxyheptanoic Acid Sodium Salt

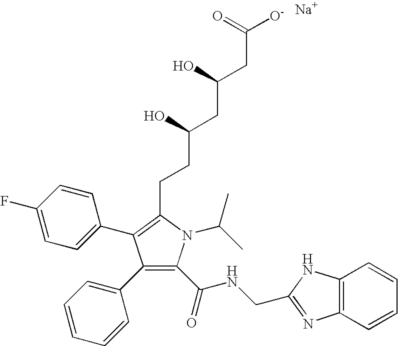

The title compound was prepared by a method analogous to that described for the preparation of Example 89, substituting 2-(aminomethyl)benzimidazole dihydrochloride hydrate for 3-(2-aminoethyl)pyridine in Step A. MS(APCI⁻) mz 611; mp 234-236° C. (dec.).

### Example 93

### (3R,5R)-7-[3-(4-Fluorophenyl)-1-isopropyl-5-[(5,6,7,8-tetrahydro-imidazo[1,2-a]pyridin-2-yl Methyl)carbamoyl]-4-phenyl-1H-pyrrol-2-yl]-3,5-dihydroxyheptanoic Acid Sodium Salt

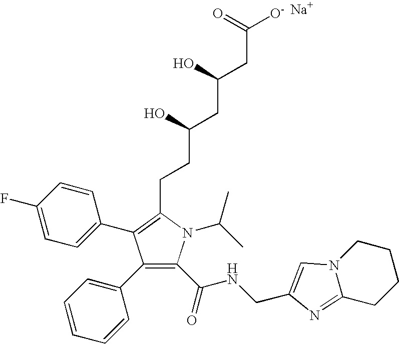

The title compound was prepared by a method analogous to that described for the preparation of Example 89, substituting 2-(aminomethyl)imidazo[1,2-a]pyridine hydrochloride (free amine commercially available) for 3-(2-aminoethyl)pyridine in Step A. MS(APCI⁺) m/z 617; mp 239-241° C. (dec.).

### Example 94

### (3R,5R)-7-[3-(4-Fluorophenyl)-1-isopropyl-5-[(1-methyl-1H-imidazol-2-yl Methyl)carbamoyl]-4-phenyl-1H-pyrrol-2-yl]-3,5-dihydroxyheptanoic Acid Sodium Salt

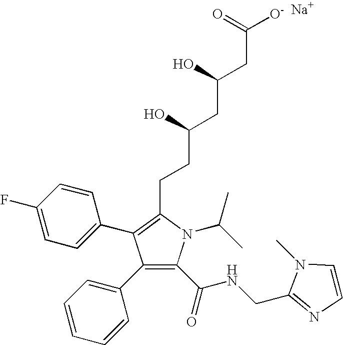

The title compound was prepared by a method analogous to that described for the preparation of Example 89, substituting 2-(aminomethyl)-1-methylimidazole dihydrochloride (WO 2004/056806) for 3-(2-aminoethyl)pyridine in Step A. NMR (400 MHz, DMSO-d₆) δ 8.24, 7.49, 7.00-6.83, 6.63, 4.74, 4.50, 4.19, 3.66, 3.52, 3.15, 2.60, 2.40, 1.92, 1.73, 1.52, 1.45, 1.38, 1.20; MS(APCI⁺) m/z 577.

### Example 95

### 3R,5R)-7-[3-(4-Fluorophenyl)-1-isopropyl-5-[(4-methyl-1H-imidazol-2-ylmethyl)carbamoyl]-4-phenyl-1H-pyrrol-2-yl]-3,5-dihydroxyheptanoic Acid Sodium Salt

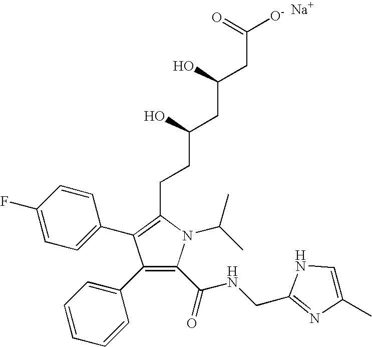

The title compound was prepared by a method analogous to that described for the preparation of Example 89, substituting 2-(aminomethyl)-4-methylimidazole dihydrochloride (free amine commercially available) for 3-(2-aminoethyl)pyridine in Step A. MS(APCI⁻) m/z 575; mp 213-215° C. (dec.).

### Example 96

### (3R,5R)-7-[3-(4-Fluorophenyl)-1-isopropyl-5-[(5-methyl-1H-pyrazol-3-yl Methyl)carbamoyl]-4-phenyl-1H-pyrrol-2-yl]-3,5-dihydroxyheptanoic Acid Sodium Salt

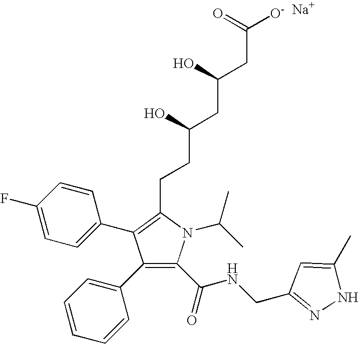

The title compound was prepared by a method analogous to that described for the preparation of Example 89, substituting 3-(aminomethyl)-5-methylpyrazole hydrochloride (free amine commercially available) for 3-(2-aminoethyl)pyridine in Step A. NMR (400 MHz, DMSO-d₆) δ 8.15, 7.55, 7.05-6.89, 5.32, 4.74, 4.50, 4.06, 3.66, 3.52, 2.63, 2.42, 2.03, 1.93, 1.73, 1.52, 1.45, 1.32, 1.20; mp 170-173° C.

### Example 97

### (3R,5R)-7-[3-(4-Fluorophenyl)-1-isopropyl-5-[(5-methylpyrazin-2-ylmethyl)carbamoyl]-4-phenyl-1H-pyrrol-2-yl]-3,5-dihydroxyheptanoic Acid Sodium Salt

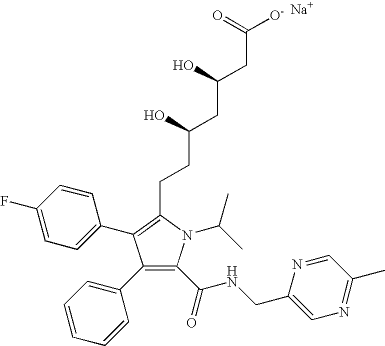 Step A

### ((4R,6S)-6-{2-[3-(4-Fluorophenyl)-1-isopropyl-4-phenyl-5-[(5-methylpyrazin-2-ylmethyl)carbamoyl]-1H-pyrrol-2-yl]vinyl)-2,2-dimethyl-[1,3]dioxan-4-yl)acetic Acid Tert-Butyl Ester

The title compound was prepared by a method analogous to that described in Steps A to D of Example 89, substituting 2-(aminomethyl)-5-methylpyrazine for 3-(2-aminoethyl)pyridine in Step A. MS(APCI⁻) m/z 681.

Step B

### cis-(3R,5S)-7-{3-(4-Fluorophenyl)-1-isopropyl-5-[(5-methylpyrazin-2-ylmethyl)carbamoyl]-4-phenyl-1H-pyrrol-2-yl}-3,5-dihydroxyhept-6-enoic Acid Tert-Butyl Ester

The title compound was prepared by a method analogous to that described in Step F of Example 89, substituting ((4R,6S)-6-{2-[3-(4-fluorophenyl)-1-isopropyl-4-phenyl-5-[(5-methylpyrazin-2-ylmethyl)carbamoyl]-1H-pyrrol-2-yl]vinyl)-2,2-dimethyl-[1,3]dioxan-4-yl)acetic acid tert-butyl ester from Step A for ((4R,6R)-6-{2-[3-(4-fluorophenyl)-1-isopropyl-4-phenyl-5-(2-pyridin-3-ylethylcarbamoyl)-1H-pyrrol-2-yl]ethyl)-2,2-dimethyl-[1,3]dioxan-4-yl)acetic acid tert-butyl ester. MS(APCI⁺) m/z 643; mp 168-170° C.

Step C

### (3R,5R)-7-{3-(4-Fluorophenyl)-1-isopropyl-5-[(5-methyl-pyrazin-2-ylmethyl)carbamoyl]-4-phenyl-1H-pyrrol-2-yl 1-3,5-dihydroxyheptanoic Acid Tert-Butyl Ester

A solution of cis-(3R,5S)-7-{3-(4-fluorophenyl)-1-isopropyl-5-[(5-methylpyrazin-2-ylmethyl)carbamoyl]-4-phenyl-1H-pyrrol-2-yl}-3,5-dihydroxyhept-6-enoic acid tert-butyl ester from Step B (0.400 g, 0.622 mmol) in methanol (20 mL) was treated with 10% palladium-on-carbon (65 mg, 0.062 mmol Pd), and the mixture was stirred under a hydrogen atmosphere (balloon) for 3 days, during which an additional 10% palladium-on-carbon (195 mg, 0.187 mmol Pd) was added in two portions. The mixture was filtered through Celite to remove the catalyst, the filtrate was concentrated in vacuo, and the residue was purified by silica gel chromatography (1-3% methanol in dichloromethane) to give 160 mg (40%) of the title compound as a white solid: mp 139-1400C; MS(APCI⁺) m/z 645.

Step D

### (3R,5R)-7-[3-(4-Fluorophenyl)-1-isopropyl-5-[(5-methylpyrazin-2-ylmethyl)carbamoyl]-4-phenyl-1H-pyrrol-2-yl]-3,5-dihydroxyheptanoic Acid Sodium Salt

The title compound was prepared by a method analogous to that described in Step G of Example 89, substituting (3R,5R)-7-{3-(4-fluorophenyl)-1-isopropyl-5-[(5-methyl-pyrazin-2-ylmethyl)carbamoyl]-4-phenyl-1H-pyrrol-2-yl}-3,5-dihydroxyheptanoic acid tert-butyl ester from Step C for (3R,5R)-7-[3-(4-fluorophenyl)-1-isopropyl-4-phenyl-5-(2-pyridin-3-ylethylcarbamoyl)-1H-pyrrol-2-yl]-3,5-dihydroxyheptanoic acid tert-butyl ester. MS(APCI⁺) m/z 589; mp 184-188° C.

### Example 98

### (3R,5R)-7-[3-(4-Fluorophenyl)-1-isopropyl-5-[(1,5-dimethyl-1H-pyrazol-3-yl Methyl)carbamoyl]-4-phenyl-1H-pyrrol-2-yl]-3,5-dihydroxyheptanoic Acid Sodium Salt

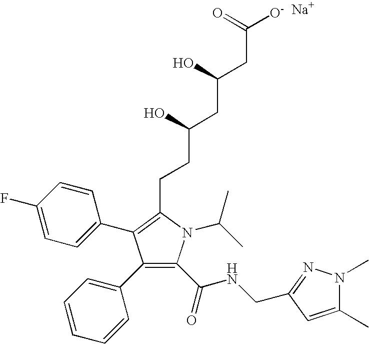 Step A

### ((4R,6S)-6-{2-[5-[(1,5-Dimethyl-1H-pyrazol-3-ylmethyl)carbamoyl]-3-(4-fluorophenyl)-1-isopropyl-4-phenyl-1H-pyrrol-2-yl]vinyl}-2,2-dimethyl-[1,3]dioxan-4-yl)acetic Acid Tert-Butyl Ester

The title compound was prepared by a method analogous to that described in Steps A to D of Example 86, substituting 3-(aminomethyl)-1,5-dimethyl-1H-pyrazole for 3-(2-aminoethyl)pyridine in Step A. MS(APCI⁺) m/z 685.

Step B

### cis-(3R,5S)-7-[5-[(1,5-Dimethyl-1H-pyrazol-3-ylmethyl)carbamoyl]-3-(4-fluorophenyl)-1-isopropyl-4-phenyl-1H-pyrrol-2-yl]-3,5-dihydroxyhept-6-enoic Acid Tert-Butyl Ester

The title compound was prepared by a method analogous to that described in Step F of Example 89, substituting ((4R,6S)-6-{2-[5-[(1,5-dimethyl-1H-pyrazol-3-ylmethyl)carbamoyl]-3-(4-fluorophenyl)-1-isopropyl-4-phenyl-1H-pyrrol-2-yl]vinyl}-2,2-dimethyl-[1,3]dioxan-4-yl)acetic acid tert-butyl ester from Step A for ((4R,6R)-6-{2-[3-(4-fluorophenyl)-1-isopropyl-4-phenyl-5-(2-pyridin-3-ylethylcarbamoyl)-1H-pyrrol-2-yl]ethyl}-2,2-dimethyl-[1,3]dioxan-4-yl)acetic acid tert-butyl ester. MS(APCI⁺) m/z 645.

Step C

### (3R,5R)-7-[5-[(1,5-Dimethyl-1H-pyrazol-3-ylmethyl)carbamoyl]-3-(4-fluorophenyl)-1-isopropyl-4-phenyl-1H-pyrrol-2-yl]-3,5-dihydroxyheptanoic Acid Tert-Butyl Ester

A solution of cis-(3R,5S)-7-[5-[(1,5-dimethyl-1H-pyrazol-3-ylmethyl)carbamoyl]-3-(4-fluorophenyl)-1-isopropyl-4-phenyl-1H-pyrrol-2-yl]-3,5-dihydroxyhept-6-enoic acid tert-butyl ester from Step B (0.375 g, 0.582 mmol) in methanol (50 mL) was treated with 10% palladium-on-carbon (0.125 g), and the mixture was shaken on a Parr apparatus under a hydrogen atmosphere (50 psi) for 2.5 hrs. The mixture was then filtered to remove the catalyst, and the filtrate was concentrated in vacuo to give 375 mg (99%) of the title compound as a glassy solid.

MS(APCI⁺) m/z 647.

Step D

### (3R,5R)-7-[3-(4-Fluorophenyl)-1-isopropyl-5-[(1,5-dimethyl-1H-pyrazol-3-ylmethyl)carbamoyl]-4-phenyl-1H-pyrrol-2-yl]-3,5-dihydroxyheptanoic Acid Sodium Salt

The title compound was prepared by a method analogous to that described in Step G of Example 89, substituting (3R,5R)-7-[5-[(1,5-dimethyl-1H-pyrazol-3-ylmethyl)carbamoyl]-3-(4-fluorophenyl)-1-isopropyl-4-phenyl-1H-pyrrol-2-yl]-3,5-dihydroxyheptanoic acid tert-butyl ester from Step C for (3R,5R)-7-[3-(4-fluorophenyl)-1-isopropyl-4-phenyl-5-(2-pyridin-3-ylethylcarbamoyl)-1H-pyrrol-2-yl]-3,5-dihydroxyheptanoic acid tert-butyl ester. NMR (400 MHz, DMSO-d₆) δ 8.06, 7.53, 7.02-6.91, 5.29, 4.74, 4.50, 4.00, 3.66, 3.52, 2.63, 2.43, 2.06, 1.93, 1.73, 1.52, 1.46, 1.32, 1.21; MS(APCI⁻) m/z 589.

### Example 99

### (3R,5R)-7-[3,4-Bis-(4-fluoro-phenyl)-1-isopropyl-5-(4-methanesulfonyl-benzylcarbamoyl)-1H-pyrrol-2-yl]-3,5-dihydroxy-heptanoic Acid, Sodium Salt

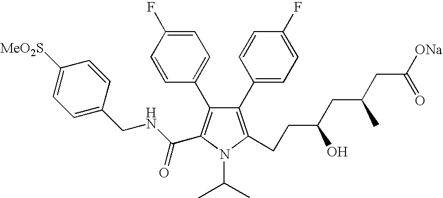 Step A

### 3,4-Bis-(4-fluoro-phenyl)-5-formyl-1-isopropyl-1H-pyrrole-2-carboxylic Acid 4-methane-sulfonyl-benzylamide

Oxalyl chloride (0.82 g, 6.5 mmol) was added dropwise to a stirred solution of 3,4-bis-(4-fluoro-phenyl)-5-formyl-1-isopropyl-1H-pyrrole-2-carboxylic acid (2.0 g, 5.4 mmol) prepared according to Example 11 Step E, in a mixture of tetrahydrofuran (20 mL) and 3-4 drops of N,N-dimethylformamide under N₂ at 0-5° C. The mixture was allowed to warm gradually to room temperature. After 2½ hours diisopropylethylamine (2.1 g, 16.2 mmol) was added, followed by 4-methanesulfonyl-benzylamine hydrochloride (1.2 g, 5.4 mmol). After 18 hours the mixture was poured into icewater (200 mL), stirred, and acidified with 4N HCl, then extracted with dichloromethane (2×75 mL). The combined organic extracts were washed with saturated aqueous sodium bicarbonate solution then brine, and dried over MgSO₄. The solvent was removed in vacuo, leaving the title compound as a cream-colored solid (3.1 g). Recrystallization from acetonitrile followed by chromatography on silica gel in 12-100% ethyl acetate in chloroform afforded a sample of analytically pure product, mp 203-204° C.; MS(APCI⁺): m/z 537 (M+H).

Step B

### (3R,5R)-7-[3,4-Bis-(4-fluoro-phenyl)-1-isopropyl-5-(4-methanesulfonyl-benzylcarbamoyl)-1H-pyrrol-2-yl]-3,5-dihydroxy-heptanoic Acid, Sodium Salt

The title compound was prepared by a method analogous to that described for the preparation of Example 4 Steps B-F, substituting 3,4-bis-(4-fluoro-phenyl)-5-formyl-1-isopropyl-1H-pyrrole-2-carboxylic acid 4-methanesulfonyl-benzylamide from Step A above for 4-(4-fluoro-phenyl)-5-formyl-1-isopropyl-3-phenyl-1H-pyrrole-2-carboxylic acid (4-sulfamoyl-phenyl)-amide. MS(APCI⁺) m/z 669 (M+2H); ¹H NMR (400 MHz, DMSO-d₆) δ 8.51 (t, 1H), 7.66 (d, 2H), 7.03-6.85 (m, 8H), 4.50 (hept, 1H), 4.25 (d, 2H), 3.73 (m, 1H), 3.54 (m, 1H), 3.12 (t, 3H), 2.68-2.00 (m, 1H), 2.03 (dd, 1H), 1.86 (dd, 1H), 1.55 (m, 1H), 1.45 (d, 6H), 1.4-1.2 (m, 3H).

### Example 100

### (3R,5R)-7-[5-(4-Dimethylcarbamoylmethyl-phenylcarbamoyl)-3,4-bis-(4-fluoro-phenyl)-1-isopropyl-1H-pyrrol-2-yl]-3,5-dihydroxy-heptanoic Acid, Sodium Salt

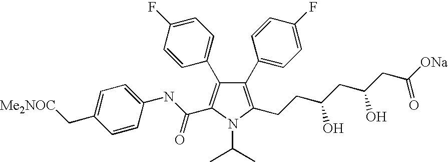 Step A

### 3,4-Bis-(4-fluoro-phenyl)-5-formyl-1-isopropyl-1H-pyrrole-2-carboxylic Acid (4-dimethylcarbamoylmethyl-phenyl)-amide

Oxalyl chloride (0.38 g, 2.98 mmol) was added to a stirred solution of 3,4-bis-(4-fluoro-phenyl)-5-formyl-1-isopropyl-1H-pyrrole-2-carboxylic acid (1.0 g, 2.7 mmol) prepared according to Example 11 Step E in a mixture of tetrahydrofuran (50 mL) and 5 drops of N,N-dimethylormamide under N₂ at 0-5° C. The mixture was allowed to warm to room temperature, and after 75 minutes was stripped of solvent under reduced pressure. The residue was dissolved in dichloromethane (25 mL) and added dropwise to a stirred solution of 2-(4-amino-phenyl)-N,N-dimethyl-acetamide (0.48 g, 2.71 mmol) and diisopropylethylamine (0.42 g, 3.25 mmol) in dichloromethane (25 mL) under N₂ at 0-5° C. The mixture was allowed to warm gradually to room temp. After 16 hours the mixture was stirred into water (60 mL), shaken thoroughly, and allowed to stratify. The layers were separated and the organic layer washed with saturated aqueous sodium bicarbonate, water, 2N HCl, and saturated brine, then dried over Mg SO₄. The solvent was removed under reduced pressure, leaving a yellow syrup which crystallized from a few drops of ethanol. The residue was recrystallized from ethanol and dried to afford the product as a snow-white powder; mp 226-227° C. Calc for C₃₁H₂₉F₂N₃O₃: C 70.31; H 5.52; N 7.93, found: C 70.14; H 5.54; N 7.86.

2-(4-Amino-phenyl)-N,N-dimethyl-acetamide is prepared according to the procedure described by McMillan, Freeman H.; Kun, Kenneth A.; McMillan, Carol B.; King, John A. Journal of the American Chemical Society (1956), 78, 4077-81.

Step B

### (3R,5R)-7-[5-(4-Dimethylcarbamoylmethyl-phenylcarbamoyl)-3,4-bis-(4-fluoro-phenyl)-1-isopropyl-1H-pyrrol-2-yl]-3,5-dihydroxy-heptanoic Acid, Sodium Salt

The title compound was prepared by a method analogous to that described for the preparation of Example 4 Steps B-F, substituting 3,4-bis-(4-fluoro-phenyl)-5-formyl-1-isopropyl-1H-pyrrole-2-carboxylic acid (4-dimethylcarbamoylmethyl-phenyl)-amide from Step A above for 4-(4-fluoro-phenyl)-5-formyl-1-isopropyl-3-phenyl-1H-pyrrole-2-carboxylic acid (4-sulfamoyl-phenyl)-amide in Step B. Mp 170-188° C.; MS(APCI⁺) m/z 660.

### Example 101

### (3R,5R)-7-[3,4-Bis-(4-fluoro-phenyl)-1-isopropyl-5-(4-sulfamoylmethyl-phenylcarbamoyl)-1H-pyrrol-2-yl]-3,5-dihydroxy-heptanoic Acid, Sodium Salt

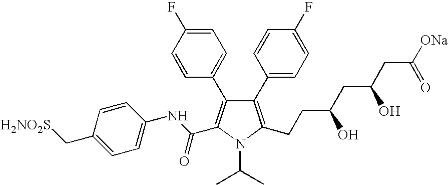

The title compound was prepared by a method analogous to that described for the preparation of Example Example 100 Steps A-B substituting (4-amino-phenyl)-methanesulfonamide for 2-(4-amino-phenyl)-N,N-dimethyl-acetamide in Step A. MS(APCI⁺) m/z 668 (M−H); ¹H NMR (400 MHz, DMSO-d₆) δ 10.07 (s, 1H), 7.39 (d, 2H), 7.17 (d, 2H), 7.0-6.9 (m, 8H), 6.72 (s, 2H), 4.55 (hept, 1H), 4.11 (s, 2H), 3.7 (m, 1H), 3.56 (m, 1H), 2.7-2.6 (m, 1H), 1.96 (dd, 1H), 1.77 (dd, 1H), 1.51 (d, 6H), 1-1.4 (m, 2H), 1.4-1.3 (m, 1H), 1.24-1.18 (m, 1H).

(4-Amino-phenyl)-methanesulfonamide is prepared according to the procedure described by Wyrick et al; Journal of Pharmaceutical Sciences, (1984), 73, 374.

### Example 102

### (3R,5R)-7-[3,4-Bis-(4-fluoro-phenyl)-1-isopropyl-5-(4-sulfamoylmethyl-phenylcarbamoyl)-1H-pyrrol-2-yl]-3,5-dihydroxy-hept-6-enoic Acid, Sodium Salt

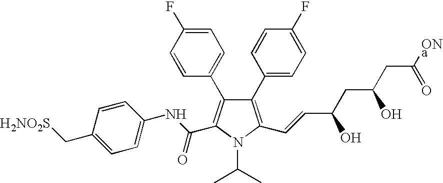

The title compound was prepared by a method analogous to Example 1 Step M, substituting (3R,5R)-7-[3,4-bis-(4-fluoro-phenyl)-1-isopropyl-5-(4-sulfamoylmethyl-phenylcarbamoyl)-1H-pyrrol-2-yl]-3,5-dihydroxy-hept-6-enoic acid methyl ester from Example 101 Step B for (3R,5R)-7-[3-(4-fluoro-phenyl)-1-isopropyl-4-phenyl-5-phenylcarbamoyl-1H-pyrrol-2-yl]-3,5-dihydroxy-heptanoic acid methyl ester. MS(APCI⁻) 666 (M−H); ¹H NMR (400 MHz, DMSO-d₆) δ 7.51 (s, 1H), 7.38 (m, 2H), 7.17 (d, 2H), 7.0-6.9 (m, 8H), 6.42 (d, 1H), 5.36 (dd, 1H), 5.0 (m, 1H), 4.64 (hept, 1H), 4.07 (m, 2H), 4.02 (s, 2H), 3.5-3.4 (m, 3H), 1.9 (dd, 1H), 1.69 (dd, 1H), 1.49 (d, 6H), 1.4-1.3 (m, 1H), 1.1-1.0 (m, 1H).

### Example 103

### (3R,5R)-7-[3,4-Bis-(4-fluoro-phenyl)-1-isopropyl-5-(4-sulfamoyl-benzylcarbamoyl)-1H-pyrrol-2-yl]-3,5-dihydroxy-heptanoic Acid, Sodium Salt

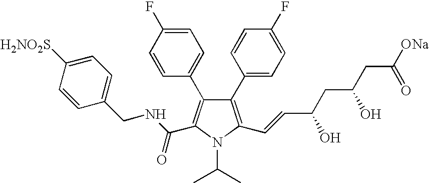

The title compound was prepared by a method analogous to that described for the preparation of Example 100 Steps A-B substituting 4-aminomethyl-benzenesulfonamide for 2-(4-amino-phenyl)-N,N-dimethyl-acetamide in Step A. MS(APCI⁺) m/z 670 (M+H); ¹H NMR (400 MHz, DMSO-d₆) δ 8.50 (t, 1H), 7.56 (d, 2H), 7.23 (bs, 2H), 7.0-6.9 (m, 10H), 4.77 (M, 1H), 4.50 (hept, 1H), 4.21 (d, 2H), 3.65 (m, 1H), 3.54 (m, 1H), 2.6 (m, 1H), 1.91 (dd, 1H), 1.71 (dd, 1H), 1.45 (d, 6H), 1.5-1.0 (m, 3H).

### Example 104

### (3R,5R)-7-[3,4-Bis-(4-fluoro-phenyl)-1-isopropyl-5-(4-sulfamoyl-benzylcarbamoyl)-1H-pyrrol-2-yl]-3,5-dihydroxy-hept-6-enoic Acid, Sodium Salt

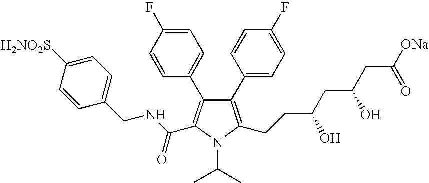

The title compound was prepared by a method analogous to Example 1 Step M, substituting (3R,5R)-7-[3,4-bis-(4-fluoro-phenyl)-1-isopropyl-5-(4-sulfamoyl-benzylcarbamoyl)-1H-pyrrol-2-yl]-3,5-dihydroxy-hept-6-enoic acid methyl ester from Example 103 Step B for (3R,5R)-7-[3-(4-fluoro-phenyl)-1-isopropyl-4-phenyl-5-phenylcarbamoyl-1H-pyrrol-2-yl]-3,5-dihydroxy-heptanoic acid methyl ester. MS(APCI⁻) 666 (M−H); ¹H NMR (400 MHz, DMSO-d₆) δ 8.55 (t, 1H), 7.57 (d, 2H), 7.46 (bs, 1H), 7.2-7.0 (bs, 2H), 7.0-6.9 (m, 10H), 6.42 (d, 1H), 6.38 (dd, 1H), 5.32 (bs, 1H), 4.57 (hept, 1H), 4.26 (d, 2H), 4.08-4.04 (m, 1H), 1.89 (dd, 1H), 1.69 (dd, 1H), 1.44 (d, 6H), 1.39-1.30 (m, 1H), 1.08-1.02 (m, 1H).

### Example 105

### (3R,5R)-7-[3,4-Bis-(4-fluoro-phenyl)-1-isopropyl-5-(4-methanesulfonylmethyl-phenylcarbamoyl)-1H-pyrrol-2-yl]-3,5-dihydroxy-heptanoic Acid, Sodium Salt

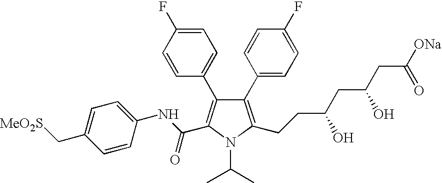 Step A

### 3,4-Bis-(4-fluoro-phenyl)-5-formyl-1-isopropyl-1H-pyrrole-2-carboxylic Acid (4-methane-sulfonylmethyl-phenyl)-amide

The title compound was prepared according to a method analogous to Example 99 Step A substituting 4-methanesulfonylmethyl-phenylamine for 4-methanesulfonyl-benzylamine hydrochloride. Mp 232-2330C; Calc for C₂₉H₂₆F₂N₂O₄S: C 64.91; H 4.88; N 5.22, found: C 64.99; H 4.61; N 5.21.

4-Methanesulfonylmethyl-phenylamine is prepared according to the procedure described in German Patent DE623883 (1936).

Step B

### (3R,5R)-7-[3,4-Bis-(4-fluoro-phenyl)-1-isopropyl-5-(4-methanesulfonylmethyl-phenylcarbamoyl)-1H-pyrrol-2-yl]-3-hydroxy-5-oxo-hept-6-enoic Acid Methyl Ester

A mixture of 3,4-bis-(4-fluoro-phenyl)-5-formyl-1-isopropyl-1H-pyrrole-2-carboxylic acid (4-methanesulfonylmethyl-phenyl)-amide (2.5 g, 4.7 mmol) from Step A and (3R)-3-(tert-butyl-dimethyl-silanyloxy)-5-oxo-6-(triphenyl-15-phosphanylidene)-hexanoic acid methyl ester (4.2 g, 7.9 mmol) was stirred in toluene (100 mL) under N₂ and heated to reflux. After 55 hours the mixture was stripped of solvent under reduced pressure, and the residue chromatographed on a column of silica gel, eluting with chloroform/ethyl acetate 4:1. The resulting crude (3R)-7-[3,4-bis-(4-fluoro-phenyl)-1-isopropyl-5-(4-methanesulfonyl methyl-phenylcarbamoyl)-1H-pyrrol-2-yl]-3-(tert-butyl-dimethyl-silanyloxy)-5-oxo-hept-6-enoic acid methyl ester was dissolved in tetrahydrofuran (25 mL), stirred under an inert atmosphere at room temperature, and a solution of 70% HF in pyridine (5.2 g, 182 mmol) was added. After one hour ice (approx 50 cc) was carefully added, followed by 1 M aqueous potassium carbonate, until the mixture was distinctly basic. The mixture was extracted with dichloromethane (2×50 mL) and the combined extracts were washed with saturated aqueous sodium bicarbonate then saturated brine, and dried over MgSO₄. The solvent was removed under reduced pressure, and the resulting residue was chromatographed on a column of silica gel, eluting with chloroform/ethyl acetate 1:1, to afford the product as a yellow powder (0.95 g) of sufficient purity for the next step.

Step C

### (3R,5R)-7-[3,4-Bis-(4-fluoro-phenyl)-1-isopropyl-5-(4-methanesulfonylmethyl-phenylcarbamoyl)-1H-pyrrol-2-yl]-3,5-dihydroxy-heptanoic Acid, Sodium Salt

The title compound was prepared by a method analogous to that described for the preparation of Example 4 Steps D-F, substituting (3R)-7-[3,4-bis-(4-fluoro-phenyl)-1-isopropyl-5-(4-methane-sulfonylmethyl-phenylcarbamoyl)-1H-pyrrol-2-yl]-3-hydroxy-5-oxo-hept-6-enoic acid methyl ester from Step B above for (3R)-7-[3-(4-fluoro-phenyl)-1-isopropyl-4-phenyl-5-(4-sulfamoyl-phenylcarbamoyl)-1H-pyrrol-2-yl]-3-hydroxy-5-oxo-hept-6-enoic acid methyl ester in Step D. MP 249° C. (dec); MS(APCI⁺) m/z 669 (M+2H).

### Example 106

### (3R,5R)-7-[3,4-Bis-(4-fluoro-phenyl)-1-isopropyl-5-(4-methanesulfonylmethyl-phenyl-carbamoyl)-1H-pyrrol-2-yl]-3,5-dihydroxy-hept-6-enoic Acid, Sodium Salt

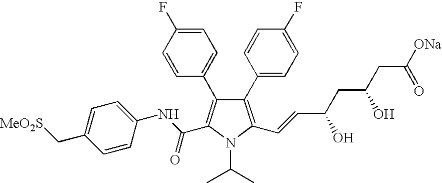

The title compound was prepared by a method analogous to Example 1 Step M, substituting (3R,5R)-7-[3,4-bis-(4-fluoro-phenyl)-1-isopropyl-5-(4-methanesulfonylmethyl-phenyl-carbamoyl)-1H-pyrrol-2-yl]-3,5-dihydroxy-hept-6-enoic acid methyl ester from Example 105 Step C for (3R,5R)-7-[3-(4-fluoro-phenyl)-1-isopropyl-4-phenyl-5-phenyl-carbamoyl-1H-pyrrol-2-yl]-3,5-dihydroxy-heptanoic acid methyl ester. MP 256° C. (dec); MS(APCI⁻) m/z 665 (M−H).

### Example 107

### (3R,5R)-7-{3-(4-Fluoro-phenyl)-1-isopropyl-4-phenyl-5-[(pyridin-2-ylmethyl)-carbamoyl]-1H-pyrrol-2-yl}-3,5-dihydroxy-heptanoic Acid, Sodium Salt

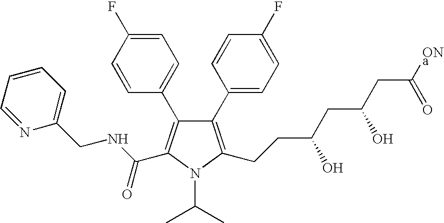

The title compound was prepared by a method analogous to that described for the preparation of Example 105 Steps A-C substituting 2-(aminomethyl)pyridine for 4-methane-sulfonylmethyl-phenylamine in Step A. MP 201-203° C.; MS(APCI⁻) m/z 572.

### Example 108

### (3R,5R)-7-[5-(3-Dimethylcarbamoyl-phenylcarbamoyl)-3-(4-fluoro-phenyl)-1-isopropyl-4-phenyl-1H-pyrrol-2-yl]-3,5-dihydroxy-heptanoic Acid, Sodium Salt

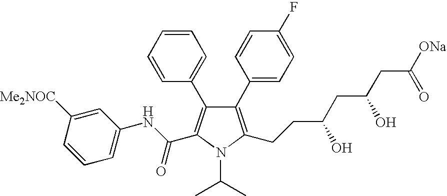 Step A

### 4-(4-Fluoro-phenyl)-5-formyl-1-isopropyl-3-phenyl-1H-pyrrole-2-carboxylic Acid (3-dimethylcarbamoyl-phenyl)-amide

Oxalyl chloride (1.4 g, 10.96 mmol) was added dropwise to a stirred solution of 4-(4-fluoro-phenyl)-5-formyl-1-isopropyl-3-phenyl-1H-pyrrole-2-carboxylic acid (3.5 g, 10 mmol) prepared according to Example 1 Step G in a mixture of tetrahydrofuran (125 mL) and ˜0.2 mL of N,N-dimethylformamide at 0-5° C. under N₂. The mixture was allowed to warm gradually to room temperature. After 3 hours the mixture was taken to dryness under reduced pressure. The residue was dissolved in of ethyl acetate (10 mL) and added dropwise to a vigorously stirred mixture of sodium carbonate (1.6 g, 15 mmol), 3-amino-N,N-dimethyl-benzamide (1.6 g, 10 mmol), H. Wenker, (JACS,60: 1080 1938),ethyl acetate (45 mL), and water (10 mL) at 0-5° C. The mixture was allowed to warm to room temperature. After 4 hours water (100 mL) was added, and the mixture filtered. The residue in the filter was rinsed with ethyl acetate then water, and air-dried, then recrystallized from acetonitrile to afford the product (3.3 g) as a yellow solid; MP 202-203° C.; sufficiently pure for the next step.

Step B

### (3R,5R)-7-[5-(3-Dimethylcarbamoyl-phenylcarbamoyl)-3-(4-fluoro-phenyl)-1-isopropyl-4-phenyl-1H-pyrrol-2-yl]-3,5-dihydroxy-heptanoic Acid, Sodium Salt

The title compound was prepared by a method analogous to that described for the preparation of Example 105 Steps B-C substituting 4-(4-fluoro-phenyl)-5-formyl-1-isopropyl-3-phenyl-1H-pyrrole-2-carboxylic acid (3-dimethylcarbamoyl-phenyl)-amide from Step A above for 3,4-bis-(4-fluoro-phenyl)-5-formyl-1-isopropyl-1H-pyrrole-2-carboxylic acid (4-methanesulfonylmethyl-phenyl)-amide in Step B. MP 156-205° C. with gas evolution; MS(APCI⁺) m/z 630 (M+H).

### Example 109

### (3R,5R)-7-[5-Benzylcarbamoyl-3,4-bis-(4-fluoro-phenyl)-1-isopropyl-1H-pyrrol-2-yl]-3,5-dihydroxy-heptanoic Acid, Sodium Salt

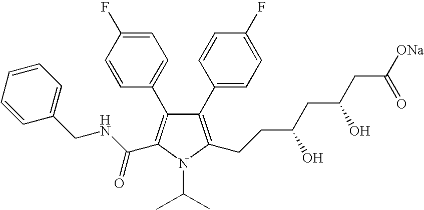

The title compound was prepared by a method analogous to that described for the preparation of Example 108 Steps A-B substituting 3,4-bis-(4-fluoro-phenyl)-5-formyl-1-isopropyl-1H-pyrrole-2-carboxylic acid prepared according to Example 11 Step E for 4-(4-fluoro-phenyl)-5-formyl-1-isopropyl-3-phenyl-1H-pyrrole-2-carboxylic acid, and substituting benzylamine for 3-amino-N,N-dimethyl-benzamide in Step A. MP 226-227° C.; MS(APCI⁻) m/z 589.

### Example 110

### (3R,5R)-7-[3,4-Bis-(4-fluoro-phenyl)-1-isopropyl-5-(3-methoxy-benzylcarbamoyl)-1H-pyrrol-2-yl]-3,5-dihydroxy-heptanoic Acid, Sodium Salt

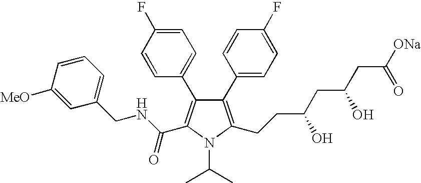 Step A

### 3,4-Bis-(4-fluoro-phenyl)-5-formyl-1-isopropyl-1H-pyrrole-2-carboxylic Acid 3-methoxy-benzylamide

The title compound was prepared by a method analogous to that described for the preparation of Example 108 Step A substituting 3,4-bis-(4-fluoro-phenyl)-5-formyl-1-isopropyl-1H-pyrrole-2-carboxylic acid prepared according to Example 11 Step E for 4-(4-fluoro-phenyl)-5-formyl-1-isopropyl-3-phenyl-1H-pyrrole-2-carboxylic acid, and substituting 3-methoxy-benzylamine for 3-amino-N,N-dimethyl-benzamide. MP 167-168° C.; Calc for C₂₉H₂₆F₂N₂O₃: C 71.30; H 5.36; N 5.73, found: C 71.05; H 5.36; N 5.65.

Step B

### (3R)-7-[3,4-Bis-(4-fluoro-phenyl)-1-isopropyl-5-(3-methoxy-benzylcarbamoyl)-1H-pyrrol-2-yl]-3-hydroxy-5-oxo-hept-6-enoic Acid Methyl Ester

The title compound was prepared by a method analogous to that described for the preparation of Example 105 Step B substituting 3,4-bis-(4-fluoro-phenyl)-5-formyl-1-isopropyl-1H-pyrrole-2-carboxylic acid 3-methoxy-benzylamide from Step A above for 3,4-bis-(4-fluoro-phenyl)-5-formyl-1-isopropyl-1H-pyrrole-2-carboxylic acid (4-methanesulfonylmethyl-phenyl)-amide. MP 104-116° C.; Calc for C₃₆H₃₆F₂N₂O₆: C 68.55; H 5.75; N 4.46, found: C 68.77; H 5.79; N 4.47.

Step C

### (3R,5R)-(6-{2-[3,4-Bis-(4-fluoro-phenyl)-1-isopropyl-5-(3-methoxy-benzylcarbamoyl)-]H-pyrrol-2-yl]-vinyl)-2-ethyl-[1,3,2]dioxaborinan-4-yl)-acetic Acid Methyl Ester

Diethyl methoxyborane (0.27 g, 2.7 mmol) was added to a stirred solution of (3R)-7-[3,4-bis-(4-fluoro-phenyl)-1-isopropyl-5-(3-methoxy-benzylcarbamoyl)-1H-pyrrol-2-yl]-3-hydroxy-5-oxo-hept-6-enoic acid methyl ester from Step B above in a mixture of tetrahydrofuran (58 mL) and methanol (14 ml) under argon at −78° C. After 15 minutes sodium borohydride (0.10 g, 2.7 mmol) was added. After 3 hours the mixture was allowed to warm gradually to room temperature. After 18 hours the mixture was recooled to <0° C., ˜2 mL of acetic acid was added, and the mixture stirred at ambient temperature. After 2 hours the mixture was poured into water (100 mL), stirred, and extracted with dichloromethane. The extract was washed with water, 0.5N sodium bicarbonate, and saturated brine, then dried over MgSO₄. The solvent was removed under reduced pressure, and the residue crystallized then recrystallized from ethanol to afford the product (0.9 g) as a brick-red powder; MP 148-149° C.; MS(APCI⁺) m/z 671.

Step D

### (3R,5R)-7-[3,4-Bis-(4-fluoro-phenyl)-1-isopropyl-5-(3-methoxy-benzylcarbamoyl)-1H-pyrrol-2-yl]-3,5-dihydroxy-heptanoic Acid Methyl Ester

Palladium on activated carbon (10%, 0.15 g) was added to a solution of (3R,5R)-(6-{2-[3,4-bis-(4-fluoro-phenyl)-1-isopropyl-5-(3-methoxy-benzylcarbamoyl)-1H-pyrrol-2-yl]-vinyl}-2-ethyl-[1,3,2]dioxaborinan-4-yl)-acetic acid methyl ester from Step C above (0.8 g, 1.2 mmol) in methanol (16 mL) and shaken at room temperature under an atmosphere of hydrogen at 40-45 psig overnight. The mixture was then filtered through Celite, the residue was rinsed with methanol, and the filtrate was stripped of solvent under reduced pressure. The residue was chromatographed on silica gel, eluting with 30-100% ethyl acetate in hexanes, to afford the product (0.32 g), of sufficient purity for the next step. MS(APCI⁺) m/z 635.

Step E

### (3R,5R)-7-[3,4-Bis-(4-fluoro-phenyl)-1-isopropyl-5-(3-methoxy-benzylcarbamoyl)-1H-pyrrol-2-yl]-3,5-dihydroxy-heptanoic Acid, Sodium Salt

The title compound was prepared by a method analogous to Example 1 Step M, substituting (3R,5R)-7-[3,4-bis-(4-fluoro-phenyl)-1-isopropyl-5-(3-methoxy-benzylcarbamoyl)-1H-pyrrol-2-yl]-3,5-dihydroxy-heptanoic acid methyl ester from Step D above for (3R,5R)-7-[3-(4-fluoro-phenyl)-1-isopropyl-4-phenyl-5-phenylcarbamoyl-1H-pyrrol-2-yl]-3,5-dihydroxy-heptanoic Acid Methyl Ester. MP 207-209° C.; MS(APCI⁺) m/z 621 (M+H).

### Example 111

### (3R,5R)-7-[3,4-Bis-(4-fluoro-phenyl)-1-isopropyl-5-(3-methoxy-benzylcarbamoyl)-1H-pyrrol-2-yl]-3,5-dihydroxy-hept-6-enoic Acid, Sodium Salt

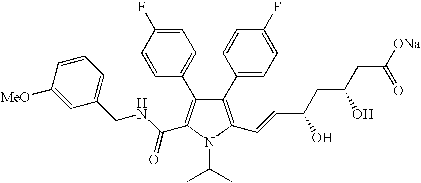 Step A

### (3R,5R)-7-[3,4-Bis-(4-fluoro-phenyl)-1-isopropyl-5-(3-methoxy-benzylcarbamoyl)-1H-pyrrol-2-yl]-3,5-dihydroxy-hept-6-enoic Acid Methyl Ester

A solution of 30% hydrogen peroxide in water (0.11 g, 0.98 mmol) was added to a stirred mixture of (3R,5R)-(6-{2-[3,4-bis-(4-fluoro-phenyl)-1-isopropyl-5-(3-methoxy-benzyl-carbamoyl)-1H-pyrrol-2-yl]-vinyl}-2-ethyl-[1,3,2]dioxaborinan-4-yl)-acetic acid methyl ester prepared according to Example 110 Steps A-C (0.66 g, 0.98 mmol) and sodium acetate (0.08 g, 0.98 mmol) in tetrahydrofuran-water 3:1 (10 mL) at room temperature. After one hour the mixture was diluted with water (100 mL) and extracted with dichloromethane (2×40 mL). The combined extracts were washed with water then saturated brine, and dried over MgSO₄. The solvent was removed under reduced pressure and the residue was chromatographed on a column of silica gel, eluting with 50-100% ethyl acetate in hexanes, to afford the product (0.6 g) as a cream-colored solid of sufficient purity for the next step. MS(APCI⁺) m/z 633.

Step B

The title compound was prepared by a method analogous to Example 1 Step M, substituting (3R,5R)-7-[3,4-bis-(4-fluoro-phenyl)-1-isopropyl-5-(3-methoxy-benzylcarbamoyl)-1H-pyrrol-2-yl]-3,5-dihydroxy-hept-6-enoic acid methyl ester from Step A above for (3R,5R)-7-[3-(4-fluoro-phenyl)-1-isopropyl-4-phenyl-5-phenylcarbamoyl-1H-pyrrol-2-yl]-3,5-dihydroxy-heptanoic acid methyl ester. MS(APCI⁺) m/z 619 (M+H); ¹H NMR (400 MHz, DMSO-d₆) δ 1.04 (m, 1H) 1.32 (m, 1H) 1.43 (d, 6H) 1.68 (dd, 1H) 1.88 (dd, 1H) 3.48 (m, 1H) 3.62 (s, 3H) 4.05 (dd, 1H) 4.15 (d, 2H) 4.56 (hept, 1H) 4.95 (m, 1H) 5.29 (dd, 1H) 6.39 (d, 1H) 6.47 (d, 1H) 6.57 (s, 1H) 6.69 (d, 1H) 6.84 (m, 2H) 6.9-7.1 (m, 7H) 8.47 (t, 1H).

### Example 112

### (3R,5R)-7-[3,4-Bis-(4-fluoro-phenyl)-1-isopropyl-5-(4-methyl-benzylcarbamoyl)-1H-pyrrol-2-yl]-3,5-dihydroxy-heptanoic Acid, Sodium Salt

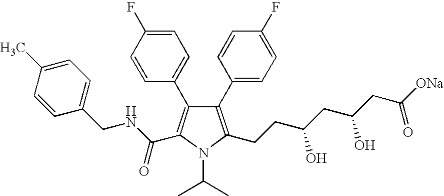

The title compound was prepared by a method analogous to Example 110 Steps A-E substituting 4-methyl-benzylamine for 3-amino-N,N-dimethyl-benzamide in Step A. MP 221-2230C; MS(APCI⁺) m/z 605 (M+H).

### Example 113

### (3R,5R)-7-[5-(4-Amino-2-oxo-pent-3-enylcarbamoyl)-3-(4-fluoro-phenyl)-1-isopropyl-4-phenyl-1H-pyrrol-2-yl]-3,5-dihydroxy-heptanoic Acid, Sodium Salt

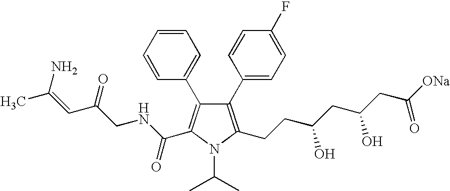 Step A

### (3R,5R)-[2-Ethyl-6-(2-{3-(4-fluoro-phenyl)-1-isopropyl-5-[(3-methyl-isoxazol-5-ylmethyl)-carbamoyl]-4-phenyl-1H-pyrrol-2-yl}-vinyl)-[1,3,2]dioxaborinan-4-yl]-acetic Acid Methyl Ester

The title compound was prepared by a method analogous to Example 110 Steps A-C substituting C-(3-methyl-isoxazol-5-yl)-methylamine for 3-amino-N,N-dimethyl-benzamide in Step A. MP 170-174° C.; MS(APCI⁺) m/z 628.

Step B

### (3R,5R)-7-[5-(4-Amino-2-oxo-pent-3-enylcarbamoyl)-3-(4-fluoro-phenyl)-1-isopropyl-4-phenyl-1H-pyrrol-2-yl]-3,5-dihydroxy-heptanoic Acid Methyl Ester

Palladium on activated carbon (10%, 0.15 g) was added to a solution of (3R,5R)-[2-ethyl-6-(2-{3-(4-fluoro-phenyl)-1-isopropyl-5-[(3-methyl-isoxazol-5-ylmethyl)-carbamoyl]-4-phenyl-1H-pyrrol-2-yl}-vinyl)-[1,3,2]dioxaborinan-4-yl]-acetic acid methyl ester from Step A above (0.85 g, 1.35 mmol) in methanol (50 mL) and shaken at room temperature under an atmosphere of hydrogen at 5-7 psig for one hour. The mixture was then filtered through Celite, the residue was rinsed with methanol, and the filtrate was stripped of solvent under reduced pressure.

The residue was recrystallized from acetonitrile to afford the product (0.33 g); MP 122-124° C.; of sufficient purity for the next step.

Step C

The title compound was prepared by a method analogous to Example 1 Step M, substituting (3R,5R)-7-[5-(4-amino-2-oxo-pent-3-enylcarbamoyl)-3-(4-fluoro-phenyl)-1-isopropyl-4-phenyl-1H-pyrrol-2-yl]-3,5-dihydroxy-heptanoic acid methyl ester from Step B above for (3R,5R)-7-[3-(4-fluoro-phenyl)-1-isopropyl-4-phenyl-5-phenylcarbamoyl-1H-pyrrol-2-yl]-3,5-dihydroxy-heptanoic acid methyl ester. MP 189-193° C.; MS(APCI⁺) m/z 580 (M+H).

### Example 114

### (3R,5R)-7-[5-Benzylcarbamoyl-3-(4-fluoro-phenyl)-1-isopropyl-4-phenyl-1H-pyrrol-2-yl]-3,5-dihydroxy-heptanoic Acid Sodium Salt

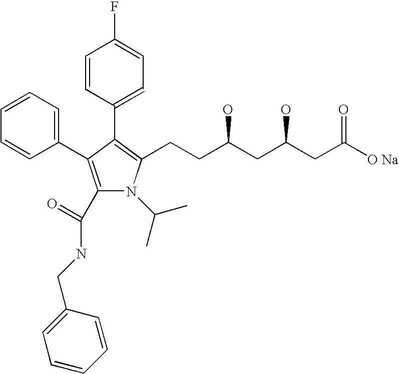 Step A

### (4R,6R)-(6-{2-[5-Benzylcarbamoyl-3-(4-fluoro-phenyl)-1-isopropyl-4-phenyl-1H-pyrrol-2-yl]-ethyl}-2,2-dimethyl-[1,3]dioxan-4-yl)-acetic Acid Methyl Ester

To a solution of (4R,6R)-(6-{2-[3-(4-fluoro-phenyl)-5-iodo-1-isopropyl-4-phenyl-1H-pyrrol-2-yl]-ethyl}-2,2-dimethyl-[1,3]dioxan-4-yl)-acetic acid methyl ester (Example 26, Step A; 0.55 g, 0.89 mmol) in dry THF (5 mL) was added benzylamine (0.39 mL, 3.6 mmol) and dichlorobis(triphenylphosphine)palladium (0.16 g, 0.22 mmol). Carbon monoxide gas was slowly bubbled in the solution while the reaction mixture was heated to reflux. The reaction mixture was stirred at reflux for 2.5 hours and the CO was bubbled in slowly for the entire reaction time. After cooling done to rt, the reaction mixture was partitioned between 1N HCl aqueous solution and EtOAc, the organic phase was washed with 1N HCl aqueous solution (2×50 mL) and brine (1×50 mL). After drying over Na₂SO₄, the organic solvent was concentrated in cacuo to give a brown solid. The solid was further purified by chromatography (1-50% EtOAc in hexanes) to give the desired product as a brown solid (0.3461 g): MS (APCI⁺) m/z 627.0 (M+H); MP 65-67° C.

Step B

### (3R,5R)-7-[5-Benzylcarbamoyl-3-(4-fluoro-phenyl)-1-isopropyl-4-phenyl-1H-pyrrol-2-yl]-3,5-dihydroxy-heptanoic Acid Methyl Ester

To a suspension of (4R,6R)-(6-{2-[5-benzylcarbamoyl-3-(4-fluoro-phenyl)-1-isopropyl-4-phenyl-1H-pyrrol-2-yl]-ethyl}-2,2-dimethyl-[1,3]dioxan-4-yl)-acetic acid methyl ester (0.31 g, 0.50 mmol) in MeOH (2 mL) was added 1 N HCl aqueous solution (0.50 mL), the resulting mixture was stirred for 18 hours. The reaction mixture was diluted with 30 mL of EtOAc, and then washed with 1 N HCl aqueous solution (2×20 mL) and brine (2×20 mL), dried over Na₂SO₄. The mixture was filtered, the filtrated was concentrated in vacuo. The residue was purified by chromatography (1-70% EtOAc in hexanes) to give the desired product as a brown foam (0.1378 g): MS (APCI⁺) m/z 587.0 (M+H); MP 52-55° C.

Step C

To a solution of 7-[5-benzylcarbamoyl-3-(4-fluoro-phenyl)-1-isopropyl-4-phenyl-1H-pyrrol-2-yl]-3,5-dihydroxy-heptanoic acid methyl ester (0.12 g, 0.21 mmol) in MeOH (3 mL) was added 1 N NaOH aqueous solution (0.23 mL, 0.23 mmol), the resulting mixture was stirred for 1 hour. The reaction mixture was concentrated in vacuo, small amount of MeOH was added followed by toluene and concentrated to dryness to azeotropically remove water, this process was repeated for three times. After further drying under vacuum, a yellow solid was obtained. 1 mL of MeOH was added, then mixed with 9 mL of CH₂Cl₂. The solution was filtered. The filtrate was concentrated affording a yellow residue, which was triturated with Et₂O to give the desired product as a yellow solid (92.2 mg): MS (APCI⁺) m/z 573.2 (M+H for the parent), MP 186-189° C.

### Example 115

### (3R,5R)-7-[3-(4-Fluoro-phenyl)-1-isopropyl-4-phenyl-5-((S)-1-phenyl-ethylcarbamoyl)-1H-pyrrol-2-yl]-3,5-dihydroxy-heptanoic Acid Sodium Salt

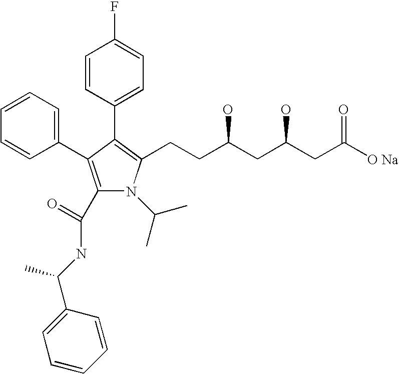 This compound was prepared in a similar manner as described for Example 114.

MS (APCI⁺) m/z 587.2 (M+H for the parent); MP 227-229° C. (decomposed).

### Example 116

### (3R,5R)-4-{[5-(6-Carboxy-3,5-dihydroxy-hexyl)-4-(4-fluoro-phenyl)-1-isopropyl-3-phenyl-1H-pyrrole-2-carbonyl]-amino}-benzoic Acid Methyl Ester Sodium Salt

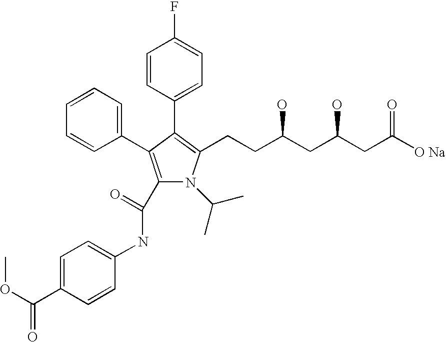 Step A

### {[4-(4-Fluoro-phenyl)-5-formyl-1-isopropyl-3-phenyl-1H-pyrrole-2-carbonyl]-amino)-benzoic Acid Methyl Ester

A mixture of 4-(4-fluoro-phenyl)-5-formyl-1-isopropyl-3-phenyl-1H-pyrrole-2-carboxylic acid (5.1 g, 14.5 mmol) and SOCl₂ (30 mL) was stirred at reflux for 50 minutes. A homogeneous solution was obtained. The reaction mixture was concentrated in vacuo, a greenish semi-solid was obtained which was dissolved in THF solution. To the THF solution was added KR (5.6 g, 42 mmol). The resulting solution was stirred at ambient temperature for 5 minutes, then a solution of 4-amino-benzoic acid methyl ester (2.2 g, 14.5 mmol) in THF was added. The resulting reaction mixture was stirred at ambient temperature for 24 hours. The reaction was quenched with 1N HCl aqueous solution, and the reaction mixture was partitioned between water and EtOAc. The organic phase was washed with 1N HCl aqueous solution and brine, dried over Mg₂SO₄. The mixture was filtered and concentrated. The crude product was purified with chromatography (5-30% EtOAc in hexanes), and then recrystallized from EtOAc/hexanes. The solid was mixed with EtOH and 0.5 mL of 1N HCl aqueous solution was added, the mixture was heated with a heatgun for 1 minute, small amount of water was added, and the mixture was cooled to ambient temperature. The mixture was filtered to give the desired product as a yellow solid (1.5671 g): MS (APCI⁺) m/z 485.0 (M+H), MP 222-223° C.

Step B

4-{[4-(4-Fluoro-phenyl)-5-formyl-1-isopropyl-3-phenyl-1H-pyrrole-2-carbonyl]-amino}-benzoic acid methyl ester was converted to (3R,5R)-4-{[5-(6-carboxy-3,5-dihydroxy-hexyl)-4-(4-fluoro-phenyl)-1-isopropyl-3-phenyl-1H-pyrrole-2-carbonyl]-amino}-benzoic acid methyl ester sodium salt in a similar manner as described for Example 1, Step I to Step M. MS (APCI⁺) m/z 617.1 (M+H for the parent); mp 188-191° C. (decomposed).

### Example 117

### (3S,5R)-4-{([5-(6-Carboxy-3,5-dihydroxy-hex-1-enyl)-4-(4-fluoro-phenyl)-1-isopropyl-3-phenyl-1H-pyrrole-2-carbonyl]-amino}-benzoic Acid Methyl Ester Sodium Salt

Starting from 4-{[4-(4-fluoro-phenyl)-5-formyl-1-isopropyl-3-phenyl-1H-pyrrole-2-carbonyl]-amino)-benzoic acid methyl ester (Example 116, Step A), this compound was prepared in a similar manner as described for Example 1 (Step I) and Example 2.

MS (APCI⁺) m/z 614.1 (APCI⁻, acid-H); mp 161-165° C. (decomposed).

### Example 118

### (3R,5R)-6-{[5-(6-Carboxy-3,5-dihydroxy-hexyl)-4-(4-fluoro-phenyl)-1-isopropyl-3-phenyl-1H-pyrrole-2-carbonyl]-amino}-nicotinic Acid Methyl Ester Sodium Salt

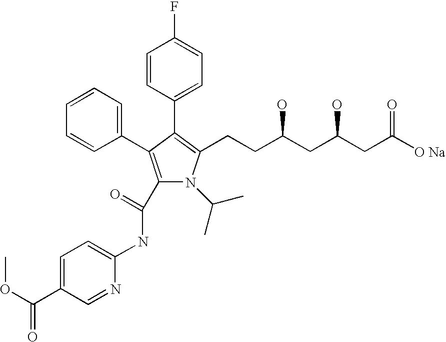

To a solution of (3R,5R)-6-{[5-(3,5-dihydroxy-6-methoxycarbonyl-hexyl)-4-(4-fluoro-phenyl)-1-isopropyl-3-phenyl-1H-pyrrole-2-carbonyl]-amino}-nicotinic acid methyl ester (Example 25, Step F, 0.92 g, 1.5 mmol) in MeOH (7 mL) was added 1 N NaOH (1.5 mL), the resulting mixture was stirred for 1.5 hours. The reaction mixture was concentrated in vacuo, small amount of MeOH was added followed by toluene and concentrated to dryness to azeotropically remove water, this process was repeated for three times. After further drying under vacuum, a white solid was obtained. 10 mL of MeOH was added, then mixed with 90 mL of CH₂Cl₂. The solution was filtered. The filtrate was concentrated affording a white residue, which was triturated with Et₂O to give the desired product as a white solid (0.9008 g): MS (APCI⁺) m/z 618.2 (M+H for the parent); MP 188-190° C. (decomposed).

### Example 119

### (3R,5R)-7-[3-(4-Fluoro-phenyl)-1-isopropyl-4-phenyl-5-(pyridin-2-ylcarbamoyl)-1 H-pyrrol-2-yl]-3,5-dihydroxy-heptanoic Acid Sodium Salt

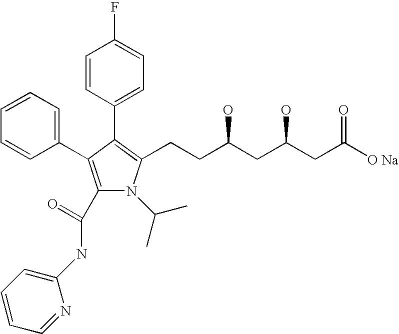 Step A

### (3R,5R)-7-[3-(4-Fluoro-phenyl)-1-isopropyl-4-phenyl-5-(pyridin-2-ylcarbamoyl)-1H-pyrrol-2-yl]-3,5-dihydroxy-heptanoic Acid Methyl Ester

This compound was prepared in a similar manner as described for Example 25 (Step C-F)

Step B

(3R,5R)-7-[3-(4-Fluoro-phenyl)-1-isopropyl-4-phenyl-5-(pyridin-2-ylcarbamoyl)-1H-pyrrol-2-yl]-3,5-dihydroxy-heptanoic acid methyl ester was converted to the desired product in a similar manner as described for Example 118. MS (APCI+) m/z 560.2 (M+H for the parent); MP 226-228° C. (decomposed).

### Example 120

### (3R,5R)-7-[3,4-Bis-(4-fluoro-phenyl)-1-isopropyl-5-(pyridin-2-ylcarbamoyl)-1H-pyrrol-2-yl]-3,5-dihydroxy-heptanoic Acid Sodium Salt

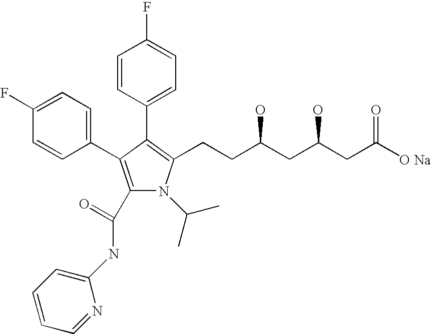 Step A

### (3R,5R)-7-[3,4-Bis-(4-fluoro-phenyl)-1-isopropyl-5-(pyridin-2-ylcarbamoyl)-1H-pyrrol-2-yl]-3,5-dihydroxy-heptanoic Acid Methyl Ester

Starting from (4R,6R)-(6-{2-[3,4-bis-(4-fluoro-phenyl)-1-isopropyl-1H-pyrrol-2-yl]-ethyl}-2,2-dimethyl-[1,3]dioxan-4-yl)-acetic acid methyl ester (Example 24), this compound was prepared in a similar manner as described for Example 25 (Step D-F).

MS (APCI+) m/z 592.2 (M+H), MP 72-75° C.

Step B

Starting from (3R,5R)-7-[3,4-bis-(4-fluoro-phenyl)-1-isopropyl-5-(pyridin-2-ylcarbamoyl)-1H-pyrrol-2-yl]-3,5-dihydroxy-heptanoic acid methyl ester, the compound was prepared in a similar manner as described for Example 118. MS (APCI+) m/z 578.2 (M+H for the parent); MP 217-219° C. (decomposed).

### Example 121

### (3R,5R)-6-{[5-(6-Carboxy-3,5-dihydroxy-hexyl)-3,4-bis-(4-fluoro-phenyl)-1-isopropyl-1H-pyrrole-2-carbonyl]-amino}-nicotinic Acid Methyl Ester Sodium Salt

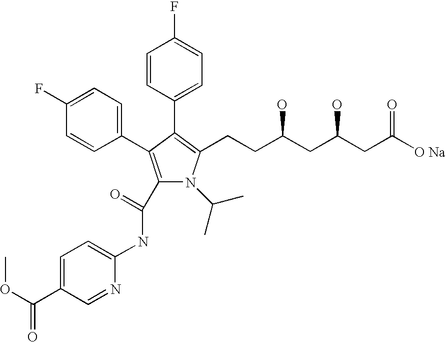 Step A

### (3R,5R)-6-{[5-(3,5-Dihydroxy-6-methoxycarbonyl-hexyl)-3,4-bis-(4-fluoro-phenyl)-1-isopropyl-1H-pyrrole-2-carbonyl]-amino-nicotinic Acid Methyl Ester

Starting from (4R,6R)-(6-{2-[3,4-Bis-(4-fluoro-phenyl)-1-isopropyl-1H-pyrrol-2-yl]-ethyl)-2,2-dimethyl-[1,3]dioxan-4-yl)-acetic acid methyl ester (Example 24), this compound was prepared in a similar manner as described for Example 25 (Step D-F).

MS (APCI⁺) m/z 650.2 (M+H), MP 158-160° C.

Step B

### (3R,5R)-6-{[5-(6-Carboxy-3,5-dihydroxy-hexyl)-3,4-bis-(4-fluoro-phenyl)-1-isopropyl-1H-pyrrole-2-carbonyl]-amino}-nicotinic Acid Methyl Ester Sodium Salt

This compound was prepared in a similar manner as described for Example 118 MS (APCI+) m/z 636.2 (M+H for the parent); MP 178-181° C. (decomposed).

### Example 122

### (3R,5R)-6-{[5-(6-Carboxy-3,5-dihydroxy-hexyl)-3,4-bis-(4-fluoro-phenyl)-1-isopropyl-1H-pyrrole-2-carbonyl]-amino}-nicotinic Acid Di-Sodium Salt

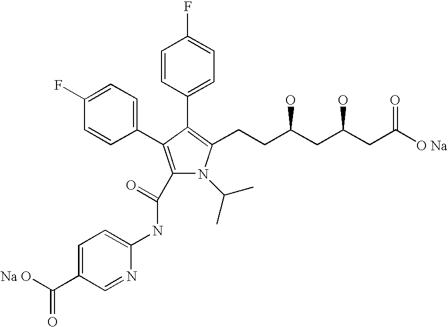

Starting from (4R,6R)-(6-{2-[3,4-bis-(4-fluoro-phenyl)-1-isopropyl-1H-pyrrol-2-yl]-ethyl}-2,2-dimethyl-[1,3]dioxan-4-yl)-acetic acid methyl ester (Example 24), this compound was prepared in a similar manner as described for Example 25.

MS (APCI+) m/z 622.2 (M+H for the parent); MP >2500C.

### Example 123

### (3R,5R)-7-[5-(Di-pyridin-2-yl-carbamoyl)-3-(4-fluoro-phenyl)-1-isopropyl-4-phenyl-1H-pyrrol-2-yl]-3,5-dihydroxy-heptanoic Acid Sodium Salt

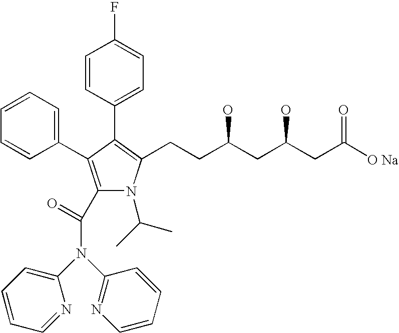 Step A

### (4R,6R)-(6-{2-[5-(Di-pyridin-2-yl-carbamoyl)-3-(4-fluoro-phenyl)-1-isopropyl-4-phenyl-1H-pyrrol-2-yl]-ethyl)-2,2-dimethyl-[1,3]dioxan-4-yl)-acetic Acid Methyl Ester

(4R,6R)-(6-{2-[5-Carbamoyl-3-(4-fluoro-phenyl)-1-isopropyl-4-phenyl-1H-pyrrol-2-yl]-ethyl}-2,2-dimethyl-[1,3]dioxan-4-yl)-acetic acid methyl ester (Example 25, Step D, 0.74 g, 1.4 mmol), 2-iodopyridine (0.34 g, 1.7 mmol), N,N′-dimethylethylenediamine (0.024 mL, 0.28 mmol), copper (I) iodide (0.026 g, 0.14 mmol), and potassium phosphate tribasic (0.58 g, 2.8 mmol) were mixed in an oven-dried flask and 0.7 mL of dry DMF was added. The resulting mixture was stirred under nitrogen at 75° C. for 15 hours. The reaction mixture was then cooled to ambient temperature and diluted with EtOAc. The mixture was then washed with water (2×50 mL), dried over Na₂SO₄, and concentrated in vacuo. The residue was purified by chromatography (1-70% EtOAc in hexanes) to give the desired product (0.14 g) as a yellow foam: MP 80-830C, MS (APCI⁺): m/z 691.2 (M+H).

Step B

### (2R,4R)-4-(4-Fluoro-phenyl)-5-[2-(4-hydroxy-6-oxo-tetrahydro-pyran-2-yl)-ethyl]-1-isopropyl-3-phenyl-1H-pyrrole-2-carboxylic Acid di-pyridin-2-yl-amide

To a solution of (4R,6R)-(6-{2-[5-(Di-pyridin-2-yl-carbamoyl)-3-(4-fluoro-phenyl)-1-isopropyl-4-phenyl-1H-pyrrol-2-yl]-ethyl}-2,2-dimethyl-[1,3]dioxan-4-yl)-acetic acid methyl ester (0.13 g, 0.19 mmol) in acetonitrile (0.5 mL) was added a solution of HF in acetonitrile (2 mL, 1:19 48% HF-acetonitrile) at rt. The mixture was stirred at ambient temperature for 4.0 h. The reaction mixture was diluted with EtOAc, the organic layer was washed with water and brine, and dried over Na₂SO₄. The mixture was filtered, the filtrate was concentrated in vacuo to give a white solid, which was purified by chromatography (1-100% EtOAc/Hexanes) to give the desired product as a white solid (0.045 g): MP 99-105° C., MS (APCI⁺) m/z 619.2 (M+H).

Step C

### To a Solution of (2R,4R)-4-(4-fluoro-phenyl)-5-[2-(4-hydroxy-6-oxo-tetrahydro-pyran-2-yl)-ethyl]-1-isopropyl-3-phenyl-1H-pyrrole-2-carboxylic Acid di-pyridin-2-yl-amide

(0.037 g, 0.060 mmol) in MeOH (1 mL) was added 1 N NaOH aqueous solution (0.06 mL), the resulting mixture was stirred for 1.5 hrs. The reaction mixture was concentrated in vacuo, small amount of MeOH was added followed by toluene and concentrated to dryness to azeotropically remove water, this process was repeated for three times. After further drying under vacuum, a yellow solid was obtained. 1 mL of MeOH was added, then mixed with 9 mL of CH₂Cl₂. The solution was filtered. The filtrate was concentrated affording a white residue, which was triturated with Et₂O to give the desired product as a white solid (35.5 mg): MS (APCI⁺) m/z 637.2 (M+H for the parent); MP 223-225° C. (decomposed).

### Example 124

### (2R,4R)-6-({4-(4-Fluoro-phenyl)-5-[2-(4-hydroxy-6-oxo-tetrahydro-pyran-2-yl)-ethyl]-1-isopropyl-3-phenyl-1H-pyrrole-2-carbonyl}-amino)-nicotinic Acid

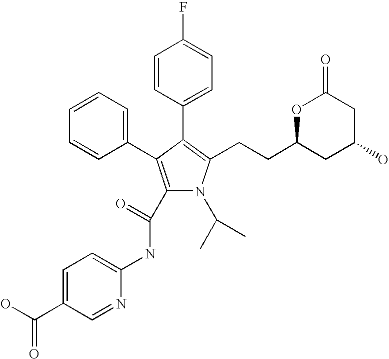

(3R,5R)-6-{[5-(6-Carboxy-3,5-dihydroxy-hexyl)-4-(4-fluoro-phenyl)-1-isopropyl-3-phenyl-1H-pyrrole-2-carbonyl]-amino}-nicotinic acid di-sodium salt (0.34 g, 0.54 mmol) was disolved in 30 mL of MeOH, and 0.37 mL of 1N HCl aqueous solution was added, the resulting reaction solution was stirred for 20 minutes and concentrated in vacuo. The residue was mixed with EtOH (10 mL), stirred for 20 minutes and filtered. The filtrated was concentrated to give the desired product as a yellow solid (0.2905 g), MS (APCI⁺) m/z 586.2 (M+H); MP 172-174° C. (decomposed).

### Example 125

### (3R,5R)-7-[3-(4-Fluoro-phenyl)-1-isopropyl-4-phenyl-5-(3-sulfamoyl-phenylcarbamoyl)-1H-pyrrol-2-yl]-3,5-dihydroxy-heptanoic Acid Sodium Salt

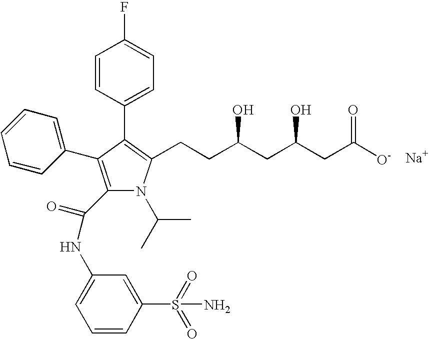 Step A

### 4-(4-Fluoro-phenyl)-5-formyl-1-isopropyl-3-phenyl-1H-pyrrole-2-carboxylic Acid (3-sulfamoyl-phenyl)-amide

A mixture of 4-(4-fluoro-phenyl)-5-formyl-1-isopropyl-3-phenyl-1H-pyrrole-2-carboxylic acid (Example 1 Step G, 1.0 g, 2.84 mmol) in thionyl chloride (5 mL) was heated at reflux for 1 h. The resulting mixture was concentrated in vacuo to give a residue, which was dried in vacuo for 1 h. The crude acid chloride was dissolved in THF (10 mL) under a nitrogen atmosphere. The mixture was cooled in an ice bath and 3-sulfamoyl-aniline (0.98 g, 5.68 mmol) was added followed by triethylamine (0.79 mL, 5.7 mmol). The mixture was stirred at room temperature overnight and partitioned between ethyl acetate and water. The organic phase was separated and washed with 1N HCl, NaHCO₃ and brine, dried over Na₂SO₄ and filtered. The filtrate was concentrated in vacuo to give a residue, which was purified by chromatography (10%-50% ethyl acetate in hexanes) to give 1.2 g (84%) of the desired product as a white solid: mp 224-225° C.; MS(APCI⁻): m/z 504.1 (M−H); Anal. Calcd for C₂₇H₂₄F₁N₃O₄S₁1.0EtOAc: C, 62.72; H, 5.43; N, 7.08. Found: C, 62.45; H, 0.33; N, 7.21.

Step B

### (3R)-7-[3-(4-Fluoro-phenyl)-1-isopropyl-4-phenyl-5-(3-sulfamoyl-phenylcarbamoyl)-1H-pyrrol-2-yl]-3-(tert-butyl-dimethyl-silanyloxy)-5-oxo-hept-6-enoic Acid Methyl Ester

To a mixture of 4-(4-fluoro-phenyl)-5-formyl-1-isopropyl-3-phenyl-1H-pyrrole-2-carboxylic acid (3-sulfamoyl-phenyl)-amide (0.9 g, 1.8 mmol) in toluene (20 mL) at room temperature under a nitrogen atmosphere was added wittig reagent [3-(tert-butyl-di methyl-silanyloxy)-5-oxo-6-(triphenyl-phosphanyl idene)-hexanoic acid methyl ester] (1.4 g, 2.7 mmol). The mixture was heated at reflux for 24 h and then concentrated in vacuo to give a residue, which was purified by chromatography (10%-50% ethyl acetate in hexanes) to give 0.53 g (39%) of the desired product as a light yellow foam: mp 90-91° C.; MS(APCI⁻): m/z 760.3 (M−H); Anal. Calcd for C₄₀H₄₈F₁N₃O₇S₁S₁.0.25EtOAc: C, 62.81; H, 6.43; N, 5.36. Found: C, 62.51; H, 6.45; N, 5.16.

Step C

### (3R)-7-[3-(4-Fluoro-phenyl)-1-isopropyl-4-phenyl-5-(3-sulfamoyl-phenylcarbamoyl)-1H-pyrrol-2-yl]-3-hydroxy-5-oxo-hept-6-enoic Acid Methyl Ester

To a solution of (3R)-7-[3-(4-fluoro-phenyl)-1-isopropyl-4-phenyl-5-(3-sulfamoyl-phenylcarbamoyl)-1H-pyrrol-2-yl]-3-(tert-butyl-dimethyl-silanyloxy)-5-oxo-hept-6-enoic acid methyl ester (560 mg, 0.74 mmol) in acetonitrile (1 mL) was added dropwise a hydrogen fluoride solution (1:19 48% HF:acetonitrile, 4 mL) in an ice bath under a nitrogen atmosphere. The mixture was stirred at room temperature for 1 h. TLC showed that the reaction was complete. The mixture was partitioned between ethyl acetate and water. The organic phase was separated and washed with NaHCO₃ and brine, dried over Na₂SO₄ and filtered. The filtrate was concentrated in vacuo to give 470 mg (99%) of the desired product as a light yellow foam: mp 89-91° C.; MS(APCI⁺): m/z 648.2 (MH⁺); Anal. Calcd for C₃₄H₃₄F₁N₃O₇S₁.0.4EtOAc: C, 62.61; H, 5.49; N, 6.15. Found: C, 62.31; H, 5.37; N, 5.87.

Step D

### (3R,5S)-7-[3-(4-Fluoro-phenyl)-1-isopropyl-4-phenyl-5-(3-sulfamoyl-phenylcarbamoyl)-1H-pyrrol-2-yl]-3,5-dihydroxy-hept-6-enoic Acid Methyl Ester

To a mixture of (3R)-7-[3-(4-fluoro-phenyl)-1-isopropyl-4-phenyl-5-(3-sulfamoyl-phenylcarbamoyl)-1H-pyrrol-2-yl]-3-hydroxy-5-oxo-hept-6-enoic acid methyl ester (448 mg, 0.70 mmol) in THF (8 mL) and methanol (2 mL) was added dropwise a solution of 1M diethyl-methoxy-borane in THF (0.76 mL) at −78° C. under a nitrogen atmosphere. The mixture was stirred for 0.5 h and then sodium borohydride (34 mg, 0.90 mmol) was added in portions. After stirring for 2 h, 2 drops of acetic acid were added. The mixture was partitioned between ethyl acetate and water. The organic phase was separated and washed with NaHCO₃ and brine, dried over Na₂SO₄ and filtered. The filtrate was concentrated in vacuo to give a residue, which was dissolved in warm methanol and concentrated in vacuo again to give a residue, which was purified by chromatography (20%-80% ethyl acetate in hexanes) to give 320 mg (71%) of the desired product as an off-white solid: mp 85-87° C.; MS(APCI⁻): m/z 649.2 (M−H); Anal. Calcd for C₃₄H₃₆F₁N₃O₇S₁.0.9H₂O: C, 61.23; H, 5.77; N, 6.23. Found: C, 61.52; H, 5.76; N, 5.84.

Step E

### (3R,5R)-7-[3-(4-Fluoro-phenyl)-1-isopropyl-4-phenyl-5-(3-sulfamoyl-phenylcarbamoyl)-1H-pyrrol-2-yl]-3,5-dihydroxy-heptanoic Acid Methyl Ester

To a solution of (3R,5S)-7-[3-(4-fluoro-phenyl)-1-isopropyl-4-phenyl-5-(3-sulfamoyl-phenylcarbamoyl)-1H-pyrrol-2-yl]-3,5-dihydroxy-hept-6-enoic acid methyl ester (230 mg, 0.35 mmol) in ethanol (10 mL) was added 10% palladium on activated carbon (60 mg). The mixture was stirred at room temperature under a hydrogen atmosphere for 3 h. TLC showed that the reaction was complete. The mixture was filtered through celite. The filtrate was concentrated in vacuo to give 225 mg (98%) white solid: mp 80-81° C.; MS(APCI⁺): m/z 652.1 (MH⁺); Anal. Calcd for C₃₄H₃₈F₁N₃O₇S₁.0.7EtOAc: C, 61.95; H, 6.16; N, 5.89. Found: C, 61.61; H, 6.00; N. 5.85.

Step F

### (3R,5R)-7-[3-(4-Fluoro-phenyl)-1-isopropyl-4-phenyl-5-(3-sulfamoyl-phenylcarbamoyl)-1H-pyrrol-2-yl]-3,5-dihydroxy-heptanoic Acid Sodium Salt

To a mixture of (3R,5R)-7-[3-(4-fluoro-phenyl)-1-isopropyl-4-phenyl-5-(3-sulfamoyl-phenylcarbamoyl)-1H-pyrrol-2-yl]-3,5-dihydroxy-heptanoic acid methyl ester (213 mg, 0.33 mmol) in a solution of absolute ethanol (2 mL) and water (0.5 mL) was added 1N sodium hydroxide solution (0.33 mL) at room temperature. The mixture was stirred for 1 h and then concentrated in vacuo to give a residue, which was dissolved in a solution of 30% methanol in methylene chloride and filtered. The filtrate was concentrated in vacuo to give a solid. The solid was triturated with diethyl ether and filtered and dried in vacuo to give 210 mg (97%) of the desired product as a white solid: mp 227-229° C.; MS(APCI⁻): m/z 638.1 (M−H); Anal. Calcd for C₃₃H₃₅F₁N₃O₇S₁Na₁.1.2H₂O: C, 58.17; H, 5.53; N, 6.17. Found: C, 58.37; H, 5.93; N, 5.81.

### Example 126

### (3R,5S)-7-[3-(4-Fluoro-phenyl)-1-isopropyl-4-phenyl-5-(3-sulfamoyl-phenylcarbamoyl)-1H-pyrrol-2-yl]-3,5-dihydroxy-hept-6-enoic Acid Sodium Salt

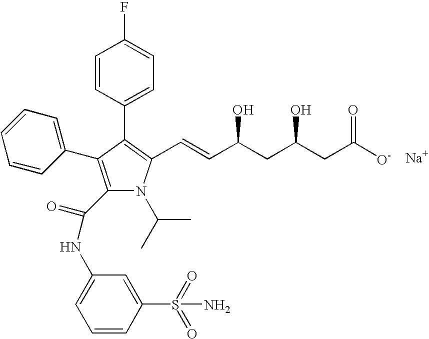

To a mixture of (3R,5S)-7-[3-(4-fluoro-phenyl)-1-isopropyl-4-phenyl-5-(3-sulfamoyl-phenylcarbamoyl)-1H-pyrrol-2-yl]-3,5-dihydroxy-hept-6-enoic acid methyl ester (Example 125, Step D; 62 mg, 0.0954 mmol) in a solution of absolute ethanol (1 mL) and water (0.5 mL) was added 1N sodium hydroxide solution (0.0954 mL) at room temperature. The mixture was stirred for 1 h and then concentrated in vacuo to give a residue, which was dissolved in a solution of 30% methanol in methylene chloride and filtered. The filtrate was concentrated in vacuo to give a solid. The solid was triturated with diethyl ether and filtered and dried in vacuo to give 62 mg (99%) of the desired product as a light yellow solid: mp 225-227° C.; MS(APCI⁻): m/z 635.1 (M−H); Anal. Calcd for C₃₃H₃₃F₁N₃O₇S₁Na₁.2.0H₂O: C, 57.13; H, 5.38; N, 6.06. Found: C, 57.02; H, 5.43; N, 5.75.

### Example 126

### (3R,5 S)-7-[3-(4-Fluoro-phenyl)-1-isopropyl-4-phenyl-5-(3-sulfamoyl-phenylcarbamoyl)-1H-pyrrol-2-yl]-3,5-dihydroxy-hept-6-enoic Acid Sodium Salt

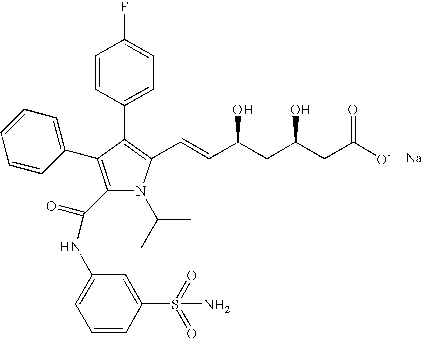

To a mixture of (3R,5S)-7-[3-(4-fluoro-phenyl)-1-isopropyl-4-phenyl-5-(3-sulfamoyl-phenylcarbamoyl)-1H-pyrrol-2-yl]-3,5-dihydroxy-hept-6-enoic acid methyl ester (Example 125, Step D; 62 mg, 0.0954 mmol) in a solution of absolute ethanol (1 mL) and water (0.5 mL) was added 1N sodium hydroxide solution (0.0954 mL) at room temperature. The mixture was stirred for 1 h and then concentrated in vacuo to give a residue, which was dissolved in a solution of 30% methanol in methylene chloride and filtered. The filtrate was concentrated in vacuo to give a solid. The solid was triturated with diethyl ether and filtered and dried in vacuo to give 62 mg (99%) of the desired product as a light yellow solid: mp 225-227° C.; MS(APCI⁻): m/z 635.1 (M−H); Anal. Calcd for C₃₃H₃₃F₁N₃O₇S₁Na₁.2.0H₂O: C, 57.13; H, 5.38; N, 6.06. Found: C, 57.02; H, 5.43; N, 5.75.

### Example 127

### 3R,5R)-7-[5-(4-Benzyloxy-phenylcarbamoyl)-3,4-bis-(4-fluoro-phenyl)-1-isopropyl-1H-pyrrol-2-yl]-3,5-dihydroxy-heptanoic Acid Sodium Salt

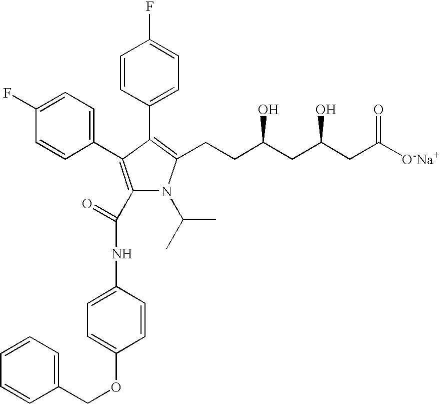

To a mixture of (3R,5R)-7-[5-(4-benzyloxy-phenylcarbamoyl)-3,4-bis-(4-fluoro-phenyl)-1-isopropyl-1H-pyrrol-2-yl]-3,5-dihydroxy-heptanoic acid methyl ester prepared in a similar manner to Example 125 step A-E (55 mg, 0.079 mmol) in a solution of absolute ethanol (1 mL) and water (0.5 mL) was added 1N sodium hydroxide solution (0.079 mL) at room temperature. The mixture was stirred for 1 h and then concentrated in vacuo to give a residue, which was dissolved in a solution of 20% methanol in methylene chloride and filtered. The filtrate was concentrated in vacuo to give a solid. The solid was triturated with diethyl ether and filtered and dried in vacuo to give 55 mg (99%) of the desired product as an off-white solid: mp 215-217° C.; MS(APCI⁺): m/z 683.3 (MH⁺); Anal. Calcd for C₄₀H₃₉F₂N₂O₆Na₁.2.5H₂O: C, 64.08; H, 5.92; N, 3.74. Found: C, 63.86; H, 5.81; N, 3.71.

### Example 128

### (3R,5S)-7-[5-(4-Benzyloxy-phenylcarbamoyl)-3,4-bis-(4-fluoro-phenyl)-1-isopropyl-1H-pyrrol-2-yl]-3,5-dihydroxy-heptanoic Acid Sodium Salt

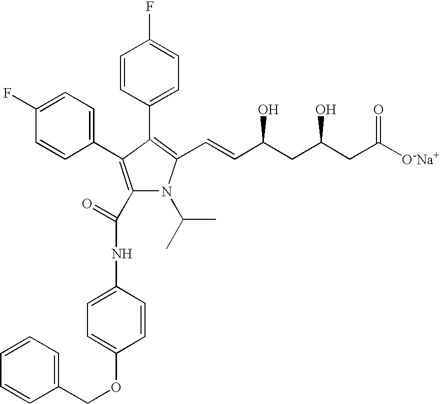

To a mixture of (3R,5S)-7-[5-(4-benzyloxy-phenylcarbamoyl)-3,4-bis-(4-fluoro-phenyl)-1-isopropyl-1H-pyrrol-2-yl]-3,5-dihydroxy-hept-6-enoic acid methyl ester prepared in a similar manner to Example 125 step A-D (113 mg, 0.163 mmol) in a solution of absolute ethanol (1 mL) and water (0.5 mL) was added 1N sodium hydroxide solution (0.163 mL) at room temperature. The mixture was stirred for 1 h and then concentrated in vacuo to give a residue, which was dissolved in a solution of 20% methanol in methylene chloride and filtered. The filtrate was concentrated in vacuo to give a solid. The solid was triturated with diethyl ether and filtered and dried in vacuo to give 114 mg (100%) of the desired product as an off-white solid: mp 218-220° C.; MS(APCI⁻): m/z 680.3 (M−H); Anal. Calcd for C₄₀H₃₇F₂N₂O₆Na₁.2.0H₂O: C, 65.03; H, 5.59; N, 3.79. Found: C, 65.27; H, 5.49; N, 3.62.

### Example 129

### (3R,5R)-7-[3,4-Bis-(4-fluoro-phenyl)-1-isopropyl-5-(4-methoxycarbonyl-benzylcarbamoyl)-1H-pyrrol-2-yl]-3,5-dihydroxy-heptanoic Sodium Salt

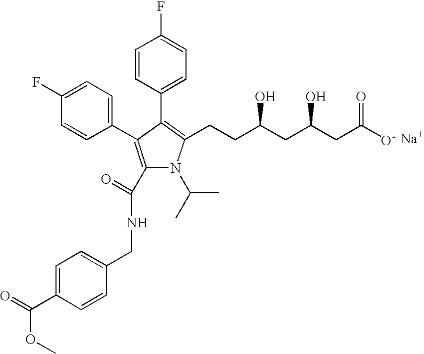

To a mixture of (3R,5R)-7-[3,4-bis-(4-fluoro-phenyl)-1-isopropyl-5-(4-methoxycarbonyl-benzylcarbamoyl)-1H-pyrrol-2-yl]-3,5-dihydroxy-heptanoic acid methyl ester prepared in a similar manner to Example 125 step A-E (137 mg, 0.207 mmol) in a solution of absolute ethanol (1 mL) and water (0.5 mL) was added 1N sodium hydroxide solution (0.207 mL) at room temperature. The mixture was stirred for 1 h and then concentrated in vacuo to give a residue, which was dissolved in a solution of 20% methanol in methylene chloride and filtered. The filtrate was concentrated in vacuo to give a solid. The solid was triturated with diethyl ether and filtered and dried in vacuo to give 137 mg (99%) of the desired product as an off-white solid: mp 188-190° C.; MS(APCI⁻): m/z 648.3 (M−H); Anal. Calcd for C₃₆H₃₇F₂N₂O₇Na₁.3.0H_{200.5}EtOH: C, 59.43; H, 6.20; N. 3.75. Found: C, 59.16; H, 6.60; N, 3.67.

### Example 130

### (3R,5S)-7-[3,4-Bis-(4-fluoro-phenyl)-1-isopropyl-5-(4-methoxycarbonyl-benzylcarbamoyl)-1H-pyrrol-2-yl]-3,5-dihydroxy-hept-6-enoic Sodium Salt

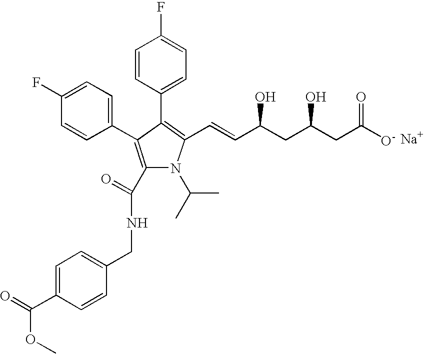

To a mixture of (3R,5S)-7-[3,4-bis-(4-fluoro-phenyl)-1-isopropyl-5-(4-methoxycarbonyl-benzylcarbamoyl)-1H-pyrrol-2-yl]-3,5-dihydroxy-hept-6-enoic acid methyl ester prepared in a similar manner to Example 125 step A-D (120 mg, 0.182 mmol) in a solution of absolute ethanol (1 mL) and water (0.5 mL) was added 1N sodium hydroxide solution (0.182 mL) at room temperature. The mixture was stirred for 1 h and then concentrated in vacuo to give a residue, which was dissolved in a solution of 20% methanol in methylene chloride and filtered. The filtrate was concentrated in vacuo to give a solid. The solid was triturated with diethyl ether and filtered and dried in vacuo to give 120 mg (99%) of the desired product as an off-white solid: mp 205-207° C.; MS(APCI⁻): m/z 646.2 (M-H); Anal. Calcd for C₃₆H₃₅F₂N₂O₇Na₁.2.0H₂O: C, 61.36; H, 5.58; N, 3.98. Found: C, 61.56; H, 5.41; N, 3.86.

### Example 131

### (3R,5S)-7-[5-(2-Benzyloxy-phenylcarbamoyl)-3,4-bis-(4-fluoro-phenyl)-1-isopropyl-1H-pyrrol-2-yl]-3,5-dihydroxy-hept-6-enoic Acid Sodium Salt

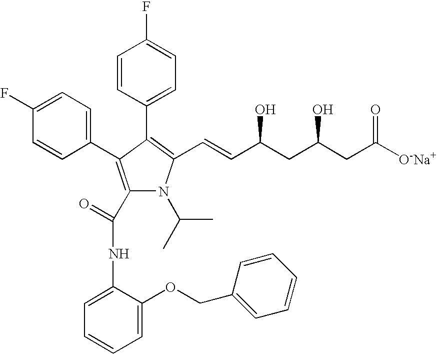

To a mixture of (3R,5S)-7-[5-(2-benzyloxy-phenylcarbamoyl)-3,4-bis-(4-fluoro-phenyl)-1-isopropyl-1H-pyrrol-2-yl]-3,5-dihydroxy-hept-6-enoic acid methyl ester prepared in a similar manner to Example 1 (KS) step A-D (180 mg, 0.259 mmol) in a solution of absolute ethanol (1 mL) and water (0.5 mL) was added 1N sodium hydroxide solution (0.259 mL) at room temperature. The mixture was stirred for 1 h and then concentrated in vacuo to give a residue, which was dissolved in a solution of 20% methanol in methylene chloride and filtered. The filtrate was concentrated in vacuo to give a solid. The solid was triturated with diethyl ether and filtered and dried in vacuo to give 180 mg (99%) of the desired product as an off-white solid: mp 228-229° C.; MS(APCI⁻): m/z 680.2 (M−H);

Anal. Calcd for C₄₀H₃₇F₂N₂O₆Na₁.1.5H₂O: C, 65.84; H, 5.52; N, 3.84. Found: C, 65.87; H, 5.52; N, 3.82.

### Example 132

### (3R,5 S)-7-[5-(3-Benzyloxy-phenylcarbamoyl)-3,4-bis-(4-fluoro-phenyl)-1-isopropyl-1H-pyrrol-2-yl]-3,5-dihydroxy-hept-6-enoic Acid Sodium Salt

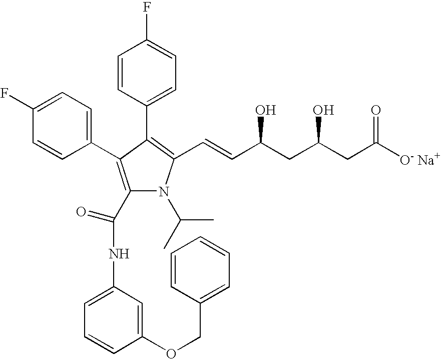

To a mixture of (3R,5S)-7-[5-(3-benzyloxy-phenylcarbamoyl)-3,4-bis-(4-fluoro-phenyl)-1-isopropyl-1H-pyrrol-2-yl]-3,5-dihydroxy-hept-6-enoic acid methyl ester prepared in a similar manner to Example 125 step A-D (147 mg, 0.212 mmol) in a solution of absolute ethanol (1 mL) and water (0.5 mL) was added 1N sodium hydroxide solution (0.212 mL) at room temperature. The mixture was stirred for 1 h and then concentrated in vacuo to give a residue, which was dissolved in a solution of 20% methanol in methylene chloride and filtered. The filtrate was concentrated in vacuo to give a solid. The solid was triturated with diethyl ether and filtered and dried in vacuo to give 147 mg (99%) of the desired product as an off-white solid: mp 225-227° C.; MS(APCI⁻): m/z 680.3 (M−H); Anal. Calcd for C₄₀H₃₇F₂N₂O₆Na₁.2.0H₂O: C, 65.03; H, 5.59; N, 3.79. Found: C, 64.91; H, 5.63; N, 3.67.

### Example 133

### (3R,5R)-7-[3,4-Bis-(4-fluoro-phenyl)-5-(4-hydroxy-phenylcarbamoyl)-1-isopropyl-1H-pyrrol-2-yl]-3,5-dihydroxy-heptanoic Acid Sodium Salt

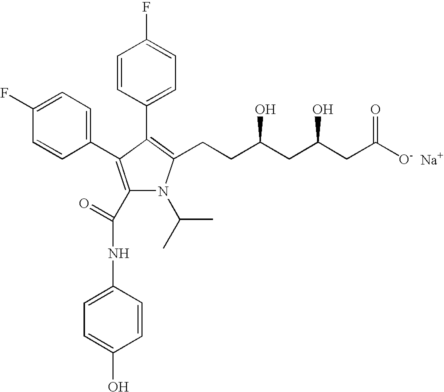

To a solution of (3R,5S)-7-[5-(4-benzyloxy-phenylcarbamoyl)-3,4-bis-(4-fluoro-phenyl)-1-isopropyl-1H-pyrrol-2-yl]-3,5-dihydroxy-hept-6-enoic acid sodium salt (Example 127); 120 mg, 0.171 mmol) in ethanol (10 mL) was added 10% palladium on activated carbon (40 mg). The mixture was stirred at room temperature under a hydrogen atmosphere for 5 h. TLC showed that the reaction was complete. The mixture was filtered through celite. The filtrate was concentrated in vacuo to give a residue, which was dissolved in a solution of 20% methanol in methylene chloride and filtered. The filtrate was concentrated in vacuo to give a solid. The solid was triturated with diethyl ether and filtered and dried in vacuo to give 80 mg (76%) white solid: mp 228-230° C.; MS(APCI⁻): m/z 591.2 (M−H); Anal. Calcd for C₃₃H₃₃F₂N₂O₆Na₁.2.5H₂O: C, 60.09; H, 5.81; N, 4.25. Found: C, 60.34; H, 5.61; N, 4.06.

### Example 134

### (3R,5R)-7-[3,4-Bis-(4-fluoro-phenyl)-5-(2-hydroxy-phenylcarbamoyl)-1-isopropyl-1H-pyrrol-2-yl]-3,5-dihydroxy-heptanoic Acid Sodium Salt

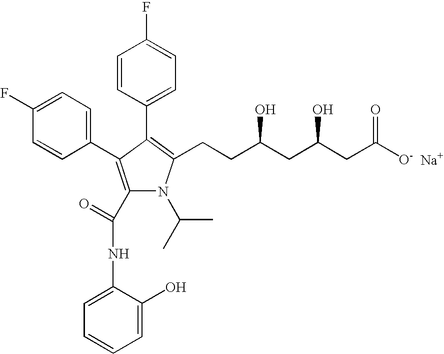

To a solution of (3R,5S)-7-[5-(2-benzyloxy-phenylcarbamoyl)-3,4-bis-(4-fluoro-phenyl)-1-isopropyl-1H-pyrrol-2-yl]-3,5-dihydroxy-hept-6-enoic acid sodium salt (Example 131); 175 mg, 0.249 mmol) in ethanol (15 mL) was added 10% palladium on activated carbon (60 mg). The mixture was stirred at room temperature under a hydrogen atmosphere for 5 h. TLC showed that the reaction was complete. The mixture was filtered through celite. The filtrate was concentrated in vacuo to give a residue, which was dissolved in a solution of 20% methanol in methylene chloride and filtered. The filtrate was concentrated in vacuo to give a solid. The solid was triturated with diethyl ether and filtered and dried in vacuo to give 120 mg (78%) white solid: mp 222-223° C.; MS(APCI⁻): m/z 592.2 (M−H); Anal. Calcd for C₃₃H₃₃F₂N₂O₆Na₁.2.0H₂O: C, 60.92; H, 5.73; N, 4.31 Found: C, 61.26; H, 5.47; N, 3.93.

### Example 135

### (3R,5R)-7-[3,4-Bis-(4-fluoro-phenyl)-5-(3-hydroxy-phenylcarbamoyl)-1-isopropyl-1H-pyrrol-2-yl]-3,5-dihydroxy-heptanoic Acid Sodium Salt

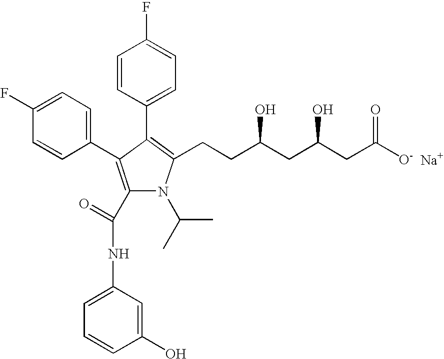

To a solution of (3R,5S)-7-[5-(3-benzyloxy-phenylcarbamoyl)-3,4-bis-(4-fluoro-phenyl)-1-isopropyl-1H-pyrrol-2-yl]-3,5-dihydroxy-hept-6-enoic acid sodium salt (Example 132); 125 mg, 0.178 mmol) in ethanol (15 mL) was added 10% palladium on activated carbon (40 mg). The mixture was stirred at room temperature under a hydrogen atmosphere for 5 h. TLC showed that the reaction was complete. The mixture was filtered through celite. The filtrate was concentrated in vacuo to give a residue, which was dissolved in a solution of 20% methanol in methylene chloride and filtered. The filtrate was concentrated in vacuo to give a solid. The solid was triturated with diethyl ether and filtered and dried in vacuo to give 100 mg (91%) white solid: mp 220-222° C.; MS(APCI⁺): m/z 593.1 (MH⁺); Anal. Calcd for C₃₃H₃₃F₂N₂O₆Na₁.1.9H₂O: C, 60.93; H, 5.85; N, 4.24. Found: C, 61.33; H, 5.98; N, 3.86.

### Example 136

### (3R,5R)-7-[3,4-Bis-(4-fluoro-phenyl)-1-isopropyl-5-(3-methoxy-phenylcarbamoyl)-1H-pyrrol-2-yl]-3,5-dihydroxy-heptanoic Acid Sodium Salt

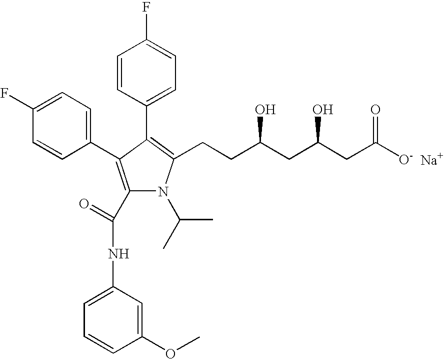

To a mixture of (3R,5R)-7-[3,4-bis-(4-fluoro-phenyl)-1-isopropyl-5-(3-methoxy-phenylcarbamoyl)-1H-pyrrol-2-yl]-3,5-dihydroxy-heptanoic acid methyl ester prepared in a similar manner to Example 125 step A-E (391 mg, 0.63 mmol) in a solution of absolute ethanol (2 mL) and water (0.5 mL) was added 1N sodium hydroxide solution (0.63 mL) at room temperature. The mixture was stirred for 1 h and then concentrated in vacuo to give a residue, which was dissolved in a solution of 20% methanol in methylene chloride and filtered. The filtrate was concentrated in vacuo to give a solid. The solid was triturated with diethyl ether and filtered and dried in vacuo to give 396 mg (100%) of the desired product as a white solid: mp 215-217° C.; MS(APCI⁻): m/z 605.2 (M−H); Anal. Calcd for C₃₄H₃₅F₂N₂O₆Na₁.2.0H₂O: C, 61.44; H, 5.91; N, 4.21. Found: C, 61.53; H, 5.98; N, 4.05.

### Example 137

### (3R,5 S)-7-[3,4-Bis-(4-fluoro-phenyl)-1-isopropyl-5-(3-methoxy-phenylcarbamoyl)-1H-pyrrol-2-yl]-3,5-dihydroxy-hept-6-enoic Acid Sodium Salt

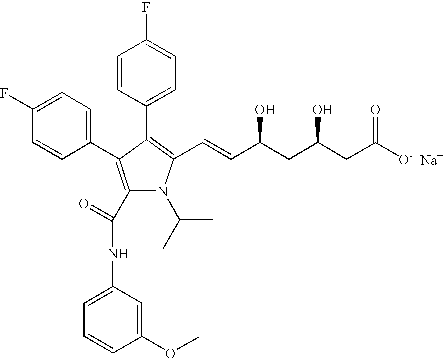

To a mixture of (3R,5S)-7-[3,4-bis-(4-fluoro-phenyl)-1-isopropyl-5-(3-methoxy-phenylcarbamoyl)-1H-pyrrol-2-yl]-3,5-dihydroxy-hept-6-enoic acid methyl ester prepared in a similar manner to Example 125 step A-D (83.3 mg, 0.135 mmol) in a solution of absolute ethanol (2 mL) and water (0.5 mL) was added 1N sodium hydroxide solution (0.135 mL) at room temperature. The mixture was stirred for 1 h and then concentrated in vacuo to give a residue, which was dissolved in a solution of 20% methanol in methylene chloride and filtered. The filtrate was concentrated in vacuo to give a solid. The solid was triturated with diethyl ether and filtered and dried in vacuo to give 83 mg (98%) of the desired product as an off-white solid: mp 225-227° C.; MS(APCI⁻): m/z 604.2 (M−H); Anal. Calcd for C₃₄H₃₃F₂N₂O₆Na₁.2.0H₂O: C, 61.63; H, 5.63; N, 4.23. Found: C, 61.65; H, 5.46; N, 4.11.

### Example 138

### (3R,5R)-7-[5-(3-Chloro-phenylcarbamoyl)-3,4-bis-(4-fluoro-phenyl)-1-isopropyl-1H-pyrrol-2-yl]-3,5-dihydroxy-heptanoic Acid Sodium Salt

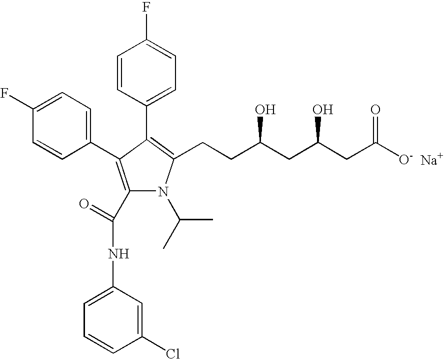

To a mixture of (3R,5R)-7-[5-(3-chloro-phenylcarbamoyl)-3,4-bis-(4-fluoro-phenyl)-1-isopropyl-1H-pyrrol-2-yl]-3,5-dihydroxy-heptanoic acid methyl ester prepared in a similar manner to Example 125 step A-E (68.7 mg, 0.107 mmol) in a solution of absolute ethanol (1 mL) and water (0.5 mL) was added 1N sodium hydroxide solution (0.107 mL) at room temperature. The mixture was stirred for 1 h and then concentrated in vacuo to give a residue, which was dissolved in a solution of 20% methanol in methylene chloride and filtered. The filtrate was concentrated in vacuo to give a solid. The solid was triturated with diethyl ether and filtered and dried in vacuo to give 68 mg (100%) of the desired product as a white solid: mp 191-194° C.; MS(APCI⁻): m/z 611.1 (M−H); Anal. Calcd for C₃₃H₃₂C₁F₂N₂O₅Na₁.0.5H₂O: C, 61.73; H, 5.18; N, 4.36. Found: C, 61.61; H, 5.34; N, 4.14.

### Example 139

### (3R,5S)-7-[5-(3-Chloro-phenylcarbamoyl)-3,4-bis-(4-fluoro-phenyl)-1-isopropyl-1H-pyrrol-2-yl]-3,5-dihydroxy-hept-6-enoic Acid Sodium Salt

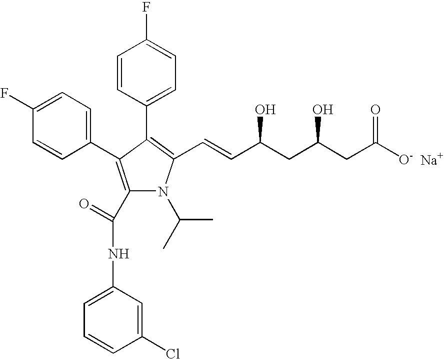

To a mixture of (3R,5S)-7-[5-(3-chloro-phenylcarbamoyl)-3,4-bis-(4-fluoro-phenyl)-1-isopropyl-1H-pyrrol-2-yl]-3,5-dihydroxy-hept-6-enoic acid methyl ester prepared in the same manner from example 125 step A-D (85.3 mg, 0.137 mmol) in a solution of absolute ethanol (1 mL) and water (0.5 mL) was added 1N sodium hydroxide solution (0.137 mL) at room temperature. The mixture was stirred for 1 h and then concentrated in vacuo to give a residue, which was dissolved in a solution of 20% methanol in methylene chloride and filtered. The filtrate was concentrated in vacuo to give a solid. The solid was triturated with diethyl ether and filtered and dried in vacuo to give 85 mg (98%) of the desired product as an off-white solid: mp 229-230° C.; MS(APCI⁻): m/z 608.1 (M−H); Anal. Calcd for C₃₃H₃₀Cl₁F₂N₂O₅Na₁.2.5H₂O: C, 58.63; H, 5.22; N, 4.14. Found: C, 58.92; H, 4.92; N, 4.05.

### Example 140

### (3R,5R)-7-[5-(3-Ethyl-phenylcarbamoyl)-3,4-bis-(4-fluoro-phenyl)-1-isopropyl-1H-pyrrol-2-yl]-3,5-dihydroxy-heptanoic Acid Sodium Salt

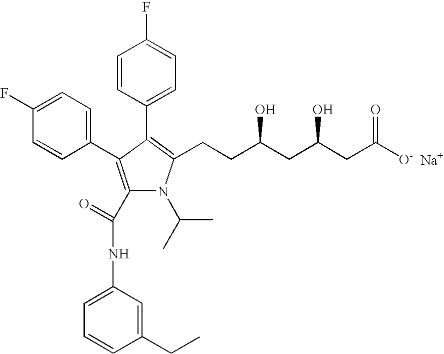

To a mixture of (3R,5R)-7-[5-(3-ethyl-phenylcarbamoyl)-3,4-bis-(4-fluoro-phenyl)-1-isopropyl-1H-pyrrol-2-yl]-3,5-dihydroxy-heptanoic acid methyl ester prepared in a similar manner to Example 125 step A-E (294 mg, 0.475 mmol) in a solution of absolute ethanol (3 mL) and water (0.5 mL) was added 1N sodium hydroxide solution (0.475 mL) at room temperature. The mixture was stirred for 1 h and then concentrated in vacuo to give a residue, which was dissolved in a solution of 20% methanol in methylene chloride and filtered. The filtrate was concentrated in vacuo to give a solid. The solid was triturated with diethyl ether and filtered and dried in vacuo to give 294 mg (99%) of the desired product as a white solid: mp 175-177° C.; MS(APCI⁻): m/z 603.2 (M−H); Anal. Calcd for C₃₅H₃₇F₂N₂O₅Na₁.2.0H₂O: C, 63.43; H, 6.24; N, 4.23. Found: C, 63.67; H, 6.08; N, 3.87.

### Example 141

### (3R,5S)-7-[5-(3-Ethyl-phenylcarbamoyl)-3,4-bis-(4-fluoro-phenyl)-1-isopropyl-1H-pyrrol-2-yl]-3,5-dihydroxy-hept-6-enoic Acid Sodium Salt

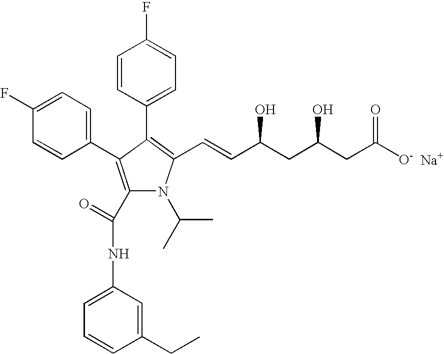

To a mixture of (3R,5S)-7-[5-(3-ethyl-phenylcarbamoyl)-3,4-bis-(4-fluoro-phenyl)-1-isopropyl-1H-pyrrol-2-yl]-3,5-dihydroxy-hept-6-enoic acid methyl ester prepared in a similar manner to Example 125 step A-D (76.9 mg, 0.125 mmol) in a solution of absolute ethanol (1 mL) and water (0.5 mL) was added 1N sodium hydroxide solution (0.125 mL) at room temperature. The mixture was stirred for 1 h and then concentrated in vacuo to give a residue, which was dissolved in a solution of 20% methanol in methylene chloride and filtered. The filtrate was concentrated in vacuo to give a solid. The solid was triturated with diethyl ether and filtered and dried in vacuo to give 77 mg (99%) of the desired product as an off-white solid: mp 229-230° C.; MS(APCI⁻): m/z 602.1 (M−H); Anal. Calcd for C₃₅H₃₅F₂N₂O₅Na₁.1.25H₂O: C, 64.96; H, 5.84; N, 4.33. Found: C, 65.24; H, 5.72; N, 4.18.

### Example 142

### (3R,5R)-7-[5-(3-Cyano-phenylcarbamoyl)-3-(4-fluoro-phenyl)-1-isopropyl-4-phenyl-1H-pyrrol-2-yl]-3,5-dihydroxy-heptanoic Acid Sodium Salt

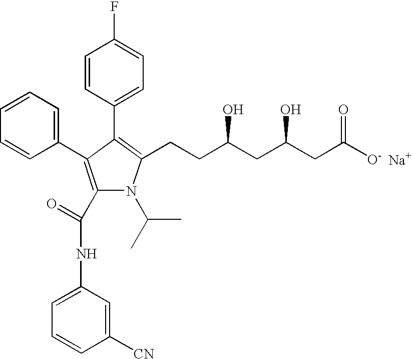

To a mixture of (3R,5R)-7-[5-(3-cyano-phenylcarbamoyl)-3-(4-fluoro-phenyl)-1-isopropyl-4-phenyl-1H-pyrrol-2-yl]-3,5-dihydroxy-heptanoic acid methyl ester prepared in a similar manner to Example 125 step A-E (192 mg, 0.321 mmol) in a solution of absolute ethanol (2 mL) and water (0.5 mL) was added 1N sodium hydroxide solution (0.321 mL) at room temperature. The mixture was stirred for 1 h and then concentrated in vacuo to give a residue, which was dissolved in a solution of 20% methanol in methylene chloride and filtered. The filtrate was concentrated in vacuo to give a solid. The solid was triturated with diethyl ether and filtered and dried in vacuo to give 194 mg (100%) of the desired product as a white solid: mp 197-199° C.; MS(APCI⁻): m/z 583.1 (M−H); Anal. Calcd for C₃₄H₃₃F₁N₃O₅Na₁.1.5H₂O: C, 64.21; H, 5.85; N, 6.42. Found: C, 64.60; H, 5.95; N, 6.02.

### Example 143

### (3R,5S)-7-[5-(3-Cyano-phenylcarbamoyl)-3-(4-fluoro-phenyl)-1-isopropyl-4-phenyl-1H-pyrrol-2-yl]-3,5-dihydroxy-hept-6-enoic Acid Sodium Salt

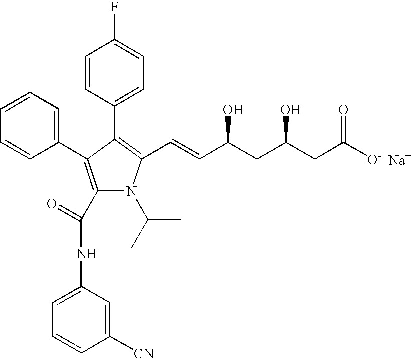

To a mixture of (3R,5S)-7-[5-(3-cyano-phenylcarbamoyl)-3-(4-fluoro-phenyl)-1-isopropyl-4-phenyl-1H-pyrrol-2-yl]-3,5-dihydroxy-hept-6-enoic acid methyl ester prepared in a similar manner to Example 125; step A-D (70 mg, 0.118 mmol) in a solution of absolute ethanol (1 mL) and water (0.5 mL) was added 1N sodium hydroxide solution (0.118 mL) at room temperature. The mixture was stirred for 1 h and then concentrated in vacuo to give a residue, which was dissolved in a solution of 20% methanol in methylene chloride and filtered. The filtrate was concentrated in vacuo to give a solid. The solid was triturated with diethyl ether and filtered and dried in vacuo to give 70 mg (99%) of the desired product as a white solid: mp 210-212° C.; MS(APCI⁻): m/z 581.1 (M−H); Anal. Calcd for C₃₄H₃₁F₁N₃O₅Na₁1.0H₂O: C, 65.69; H, 5.35; N, 6.76. Found: C, 65.93; H, 5.17; N, 6.59.

### Example 144

### (3R,5R)-7-[5-(4-Cyano-benzylcarbamoyl)-3-(4-fluoro-phenyl)-1-isopropyl-4-phenyl-1H-pyrrol-2-yl]-3,5-dihydroxy-heptanoic Acid Sodium Salt

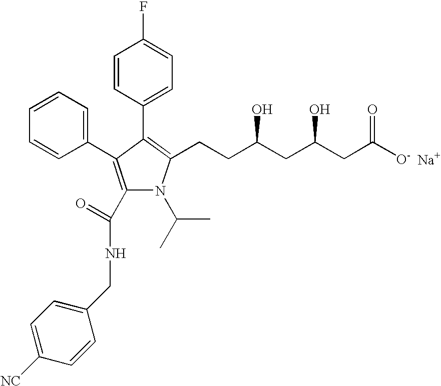

To a mixture of (3R,5R)-7-[5-(4-cyano-benzylcarbamoyl)-3-(4-fluoro-phenyl)-1-isopropyl-4-phenyl-1H-pyrrol-2-yl]-3,5-dihydroxy-heptanoic acid methyl ester prepared in a similar manner to Example 125 step A-E (317 mg, 0.518 mmol) in a solution of absolute ethanol (2 mL) and water (0.5 mL) was added 1N sodium hydroxide solution (0.518 mL) at room temperature. The mixture was stirred for 1 h and then concentrated in vacuo to give a residue, which was dissolved in a solution of 20% methanol in methylene chloride and filtered. The filtrate was concentrated in vacuo to give a solid. The solid was triturated with diethyl ether and filtered and dried in vacuo to give 320 mg (100%) of the desired product as a white solid: mp 178-180° C.; MS(APCI⁻): m/z 596.2 (M−H); Anal. Calcd for C₃₅H₃₅F₁N₃O₅Na₁.0.75H₂O: C, 66.39; H, 5.81; N, 6.64. Found: C, 66.41; H, 5.94; N, 6.34.

### Example 145

### (3R,5S)-7-[5-(4-Cyano-benzylcarbamoyl)-3-(4-fluoro-phenyl)-1-isopropyl-4-phenyl-1H-pyrrol-2-yl]-3,5-dihydroxy-hept-6-enoic Acid Sodium Salt

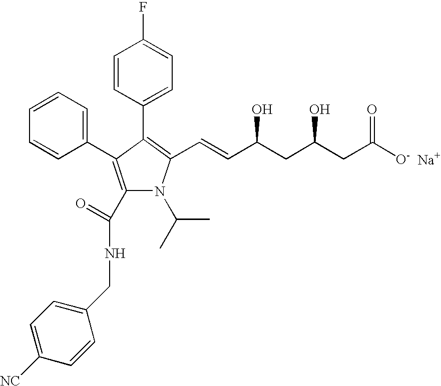

To a mixture of (3R,5S)-7-[5-(4-cyano-benzylcarbamoyl)-3-(4-fluoro-phenyl)-1-isopropyl-4-phenyl-1H-pyrrol-2-yl]-3,5-dihydroxy-hept-6-enoic acid methyl ester prepared in a similar manner to Example 125 step A-D (79 mg, 0.130 mmol) in a solution of absolute ethanol (1 mL) and water (0.5 mL) was added 1N sodium hydroxide solution (0.130 mL) at room temperature. The mixture was stirred for 1 h and then concentrated in vacuo to give a residue, which was dissolved in a solution of 20% methanol in methylene chloride and filtered. The filtrate was concentrated in vacuo to give a solid. The solid was triturated with diethyl ether and filtered and dried in vacuo to give 80 mg (100%) of the desired product as a white solid: mp 193-195° C.; MS(APCI⁻): m/z 595.2 (M−H); Anal. Calcd for C₃₅H₃₃F₁N₃O₅Na₁.1.0H₂O: C, 66.13; H, 5.55; N, 6.61. Found: C, 65.82; H, 5.51; N, 6.44.

### Example 146

### (3R,5R)-7-[5-(3-Cyano-benzylcarbamoyl)-3-(4-fluoro-phenyl)-1-isopropyl-4-phenyl-1H-pyrrol-2-yl]-3,5-dihydroxy-heptanoic Acid Sodium Salt

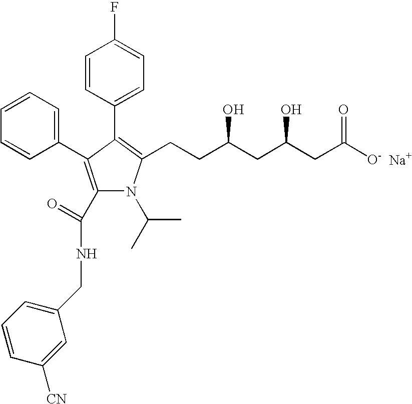

To a mixture of (3R,5R)-7-[5-(3-cyano-benzylcarbamoyl)-3-(4-fluoro-phenyl)-1-isopropyl-4-phenyl-1H-pyrrol-2-yl]-3,5-dihydroxy-heptanoic acid methyl ester prepared in a similar manner to Example 125 step A-E (328 mg, 0.536 mmol) in a solution of absolute ethanol (3 mL) and water (0.5 mL) was added 1N sodium hydroxide solution (0.536 mL) at room temperature. The mixture was stirred for 1 h and then concentrated in vacuo to give a residue, which was dissolved in a solution of 20% methanol in methylene chloride and filtered. The-filtrate was concentrated in vacuo to give a solid. The solid was triturated with diethyl ether and filtered and dried in vacuo to give 330 mg (99%) of the desired product as a white solid: mp 180-182° C.; MS(APCI⁻): m/z 597.2 (M−H); Anal. Calcd for C₃₅H₃₅F₁N₃O₅Na₁.0.85H₂O: C, 66.20; H, 5.83; N, 6.62. Found: C, 66.45; H, 5.88; N, 6.22.

### Example 147

### (3R,5S)-7-[5-(3-Cyano-benzylcarbamoyl)-3-(4-fluoro-phenyl)-1-isopropyl-4-phenyl-1H-pyrrol-2-yl]-3,5-dihydroxy-hept-6-enoic Acid Sodium Salt

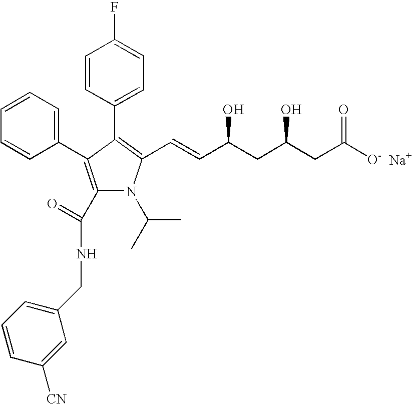

To a mixture of (3R,5S)-7-[5-(3-cyano-benzylcarbamoyl)-3-(4-fluoro-phenyl)-1-isopropyl-4-phenyl-1H-pyrrol-2-yl]-3,5-dihydroxy-hept-6-enoic acid methyl ester prepared in a similar manner to Example 125 step A-D (76 mg, 0.125 mmol) in a solution of absolute ethanol (1 mL) and water (0.5 mL) was added 1N sodium hydroxide solution (0.125 mL) at room temperature. The mixture was stirred for 1 h and then concentrated in vacuo to give a residue, which was dissolved in a solution of 20% methanol in methylene chloride and filtered. The filtrate was concentrated in vacuo to give a solid. The solid was triturated with diethyl ether and filtered and dried in vacuo to give 77 mg (100%) of the desired product as a white solid: mp 208-210° C.; MS(APCI⁻): m/z 595.2 (M−H); Anal. Calcd for C₃₅H₃₃F₁N₃O₅Na₁1.2H₂O: C, 65.76; H, 5.58; N, 6.57. Found: C, 65.79;H, 5.56; N, 6.44.

### Example 148

### (3R,5R)-7-[3-(4-Fluoro-phenyl)-5-(4-isopropoxycarbonyl-benzylcarbamoyl)-1-isopropyl-4-phenyl-1H-pyrrol-2-yl]-3,5-dihydroxy-heptanoic Acid Sodium Salt

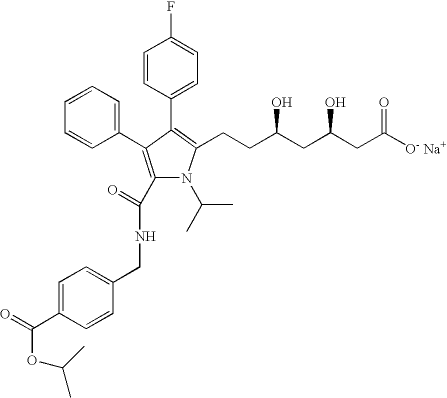

To a mixture of (3R,5R)-7-[3-(4-fluoro-phenyl)-5-(4-isopropoxycarbonyl-benzylcarbamoyl)-isopropyl-4-phenyl-1H-pyrrol-2-yl]-3,5-dihydroxy-heptanoic acid methyl ester prepared in a similar manner to Example 1 (KS) step A-E (447 mg, 0.664 mmol) in a solution of absolute ethanol (3 mL) and water (0.5 mL) was added 1N sodium hydroxide solution (0.664 mL) at room temperature. The mixture was stirred for 1 h and then concentrated in vacuo to give a residue, which was dissolved in a solution of 20% methanol in methylene chloride and filtered. The filtrate was concentrated in vacuo to give a solid. The solid was triturated with diethyl ether and filtered and dried in vacuo to give 450 mg (99%) of the desired product as a white solid: mp 199-201° C.; MS(APCI⁻): m/z 657.3 (M−H); Anal. Calcd for C₃₈H₄₂F₁N₂O₇Na₁.1.0H₂O: C, 65.32; H, 6.35; N, 4.01. Found: C, 65.66; H, 6.50; N, 3.93.

### Example 149

### (3R,5S)-7-[3-(4-Fluoro-phenyl)-5-(4-isopropoxycarbonyl-benzylcarbamoyl)-1-isopropyl-4-phenyl-1H-pyrrol-2-yl]-3,5-dihydroxy-hept-6-enoic Acid Sodium Salt

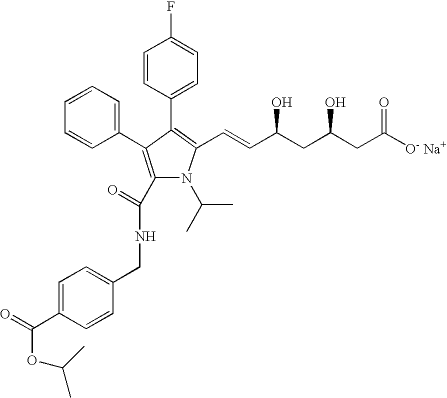

To a mixture of (3R,5S)-7-[3-(4-fluoro-phenyl)-5-(4-isopropoxycarbonyl-benzylcarbamoyl)-1-isopropyl-4-phenyl-1H-pyrrol-2-yl]-3,5-dihydroxy-hept-6-enoic acid methyl ester prepared in a similar manner to Example 125 step A-D (81.3 mg, 0.121 mmol) in a solution of absolute ethanol (1 mL) and water (0.5 mL) was added 1N sodium hydroxide solution (0.121 mL) at room temperature. The mixture was stirred for 1 h and then concentrated in vacuo to give a residue, which was dissolved in a solution of 20% methanol in methylene chloride and filtered. The filtrate was concentrated in vacuo to give a solid. The solid was triturated with diethyl ether and filtered and dried in vacuo to give 82 mg (100%) of the desired product as an off-white solid: mp 222-224° C.; MS(APCI⁻): m/z 656.3 (M−H); Anal. Calcd for C₃₈H₄₀F₁N₂O₇Na₁.1.0H₂O: C, 65.51; H, 6.08; N, 4.02. Found: C, 65.64; H, 6.04; N, 3.87.

### Example 150

### (3R,5R)-7-[3-(4-Fluoro-phenyl)-1-isopropyl-5-(4-methoxycarbonyl-benzylcarbamoyl)-4-phenyl-1H-pyrrol-2-yl]-3,5-dihydroxy-heptanoic Acid Sodium Salt

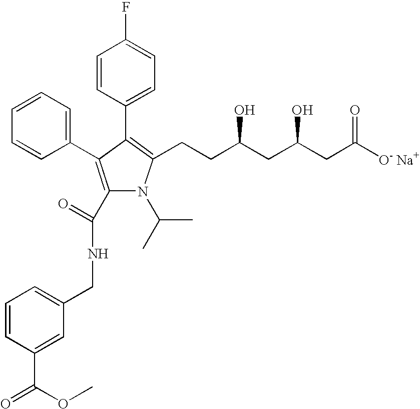

To a mixture of (3R,5R)-7-[3-(4-fluoro-phenyl)-1-isopropyl-5-(3-methoxycarbonyl-benzylcarbamoyl)-4-phenyl-1H-pyrrol-2-yl]-3,5-dihydroxy-heptanoic acid methyl ester prepared in a similar manner to Example 125 step A-E (464 mg, 0.719 mmol) in a solution of absolute ethanol (3 mL) and water (0.5 mL) was added 1N sodium hydroxide solution (0.719 mL) at room temperature. The mixture was stirred for 1 h and then concentrated in vacuo to give a residue, which was dissolved in a solution of 20% methanol in methylene chloride and filtered. The filtrate was concentrated in vacuo to give a solid. The solid was triturated with diethyl ether and filtered and dried in vacuo to give 466 mg (99%) of the desired product as a white solid: mp 183-185° C.; MS(APCI⁻): m/z 629.2 (M−H); Anal. Calcd for C₃₆H₃₈F₁N₂O₇Na₁.1.0H₂O: C, 64.47; H, 6.01; N, 4.18. Found: C, 64.76; H, 5.93; N, 4.05.

### Example 151

### (3R,5S)-7-[3-(4-Fluoro-phenyl)-1-isopropyl-5-(3-methoxycarbonyl-benzylcarbamoyl)-4-phenyl-1H-pyrrol-2-yl]-3,5-dihydroxy-hept-6-enoic Acid Sodium Salt

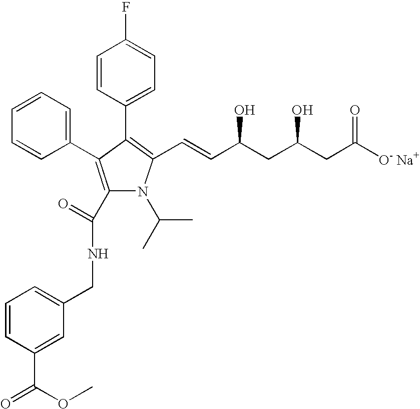

To a mixture of (3R,5S)-7-[3-(4-fluoro-phenyl)-1-isopropyl-5-(3-methoxycarbonyl-benzylcarbamoyl)-4-phenyl-1H-pyrrol-2-yl]-3,5-dihydroxy-hept-6-enoic acid methyl ester prepared in a similar manner to Example 125 step A-D (79.1 mg, 0.123 mmol) in a solution of absolute ethanol (1 mL) and water (0.5 mL) was added 1N sodium hydroxide solution (0.123 mL) at room temperature. The mixture was stirred for 1 h and then concentrated in vacuo to give a residue, which was dissolved in a solution of 20% methanol in methylene chloride and filtered. The filtrate was concentrated in vacuo to give a solid. The solid was triturated with diethyl ether and filtered and dried in vacuo to give 80 mg (100%) of the desired product as a white solid: mp 171-174° C.; MS(APCI⁻): m/z 628.2 (M−H); Anal. Calcd for C₃₆H₃₆F₁N₂O₇Na₁.0.5H₂O: C, 65.55; H, 5.65; N, 4.25. Found: C, 65.70; H, 5.77; N, 4.07.

### Example 152

### (3R,5R)-7-{3,4-Bis-(4-fluoro-phenyl)-1-isopropyl-5-[(S)—I-(4-methoxy-phenyl)-ethylcarbamoyl]-1H-pyrrol-2-yl]-3,5-dihydroxy-heptanoic Acid Sodium Salt

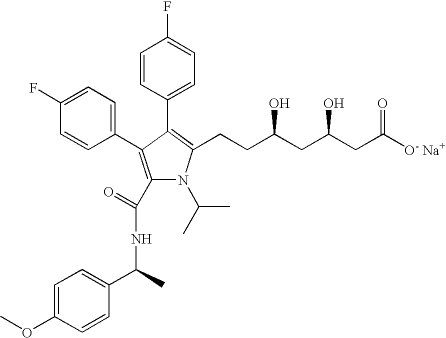

To a mixture of (3R,5R)-7-{3,4-Bis-(4-fluoro-phenyl)-1-isopropyl-5-[(S)-1-(4-methoxy-phenyl)-ethylcarbamoyl]-1H-pyrrol-2-yl)-3,5-dihydroxy-heptanoic acid methyl ester prepared in a similar manner to Example 125 step A-E (291 mg, 0.449 mmol) in a solution of absolute ethanol (3 mL) and water (0.5 mL) was added 1N sodium hydroxide solution (0.449 mL) at room temperature. The mixture was stirred for 1 h and then concentrated in vacuo to give a residue, which was dissolved in a solution of 20% methanol in methylene chloride and filtered. The filtrate was concentrated in vacuo to give a solid. The solid was triturated with diethyl ether and filtered and dried in vacuo to give 294 mg (100%) of the desired product as a white solid: mp 227-229° C.; MS(APCI⁻): m/z 633.2 (M−H); Anal. Calcd for C₃₆H₃₉F₂N₂O₆Na₁.1.7H₂O: C, 62.91; H, 6.22; N, 4.08. Found: C, 62.86; H, 6.12; N, 4.03.

### Example 153

### (3R,5S,1′S)-7-{3,4-Bis-(4-fluoro-phenyl)-1-isopropyl-5-[1-(4-methoxy-phenyl)-ethylcarbamoyl]-1H-pyrrol-2-yl}-3,5-dihydroxy-hept-6-enoic Acid Sodium Salt

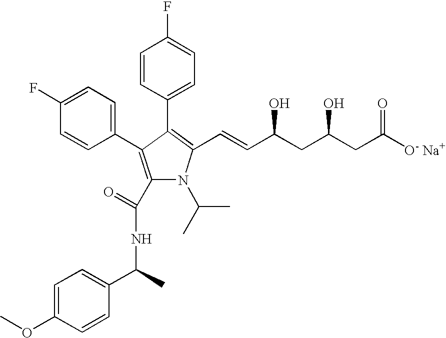

To a mixture of (3R,5S,1′S)-7-{3,4-Bis-(4-fluoro-phenyl)-1-isopropyl-5-[1-(4-methoxy-phenyl)-ethylcarbamoyl]-1H-pyrrol-2-yl}-3,5-dihydroxy-hept-6-enoic acid methyl ester prepared in a similar manner to Example 125 step A-D (225 mg, 0.348 mmol) in a solution of absolute ethanol (3 mL) and water (0.5 mL) was added 1N sodium hydroxide solution (0.348 mL) at room temperature. The mixture was stirred for 1 h and then concentrated in vacuo to give a residue, which was dissolved in a solution of 20% methanol in methylene chloride and filtered. The filtrate was concentrated in vacuo to give a solid. The solid was triturated with diethyl ether and filtered and dried in vacuo to give 228 mg (100%) of the desired product as a white solid: mp 238-240° C.; MS(APCI⁻): m/z 632.2 (M−H); Anal. Calcd for C₃₆H₃₇F₂N₂O₆Na₁.1.75H₂O: C, 63.01; H, 5.95; N, 4.08. Found: C, 63.04; H, 5.73; N, 4.02.

### Example 154

### (3R,5R)-7-{3,4-Bis-(4-fluoro-phenyl)-1-isopropyl-5-[(R)-1-(4-methoxy-phenyl)-ethylcarbamoyl]-1H-pyrrol-2-yl}-3,5-dihydroxy-heptanoic Acid Sodium Salt

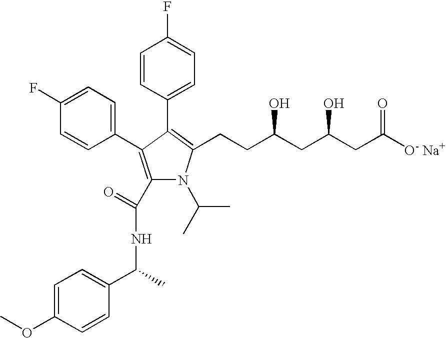

To a mixture of (3R,5R)-7-{3,4-Bis-(4-fluoro-phenyl)-1-isopropyl-5-[(R)-1-(4-methoxy-phenyl)-ethylcarbamoyl]-1H-pyrrol-2-yl)-3,5-dihydroxy-heptanoic acid methyl ester prepared in a similar manner to Example 125 step A-E (511 mg, 0.788 mmol) in a solution of absolute ethanol (4 mL) and water (0.5 mL) was added 1N sodium hydroxide solution (0.788 mL) at room temperature. The mixture was stirred for 1 h and then concentrated in vacuo to give a residue, which was dissolved in a solution of 20% methanol in methylene chloride and filtered. The filtrate was concentrated in vacuo to give a solid. The solid was triturated with diethyl ether and filtered and dried in vacuo to give 517 mg (100%) of the desired product as a white solid: mp 225-227° C.; MS(APCI⁻): m/z 633.2 (M−H); Anal. Calcd for C₃₆H₃₉F₂N₂O₆Na₁.1.7H₂O: C, 62.91; H, 6.22; N, 4.08. Found: C, 62.81; H, 6.24; N, 4.04.

### Example 155

### (3R,5S)-7-{3,4-Bis-(4-fluoro-phenyl)-1-isopropyl-5-[(R)-1-(4-methoxy-phenyl)-ethylcarbamoyl]-1H-pyrrol-2-yl)-3,5-dihydroxy-hept-6-enoic Acid Sodium Salt

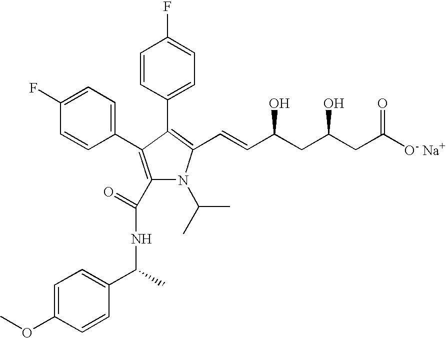

To a mixture of (3R,5S)-7-{3,4-Bis-(4-fluoro-phenyl)-1-isopropyl-5-[(R)-1-(4-methoxy-phenyl)-ethylcarbamoyl]-1H-pyrrol-2-yl)-3,5-dihydroxy-hept-6-enoic acid methyl ester prepared in a similar manner to Example 125 step A-D (306 mg, 0.473 mmol) in a solution of absolute ethanol (3 mL) and water (0.5 mL) was added 1N sodium hydroxide solution (0.473 mL) at room temperature. The mixture was stirred for 1 h and then concentrated in vacuo to give a residue, which was dissolved in a solution of 20% methanol in methylene chloride and filtered. The filtrate was concentrated in vacuo to give a solid. The solid was triturated with diethyl ether and filtered and dried in vacuo to give 309 mg (100%) of the desired product as a white solid: mp 255-257° C.; MS(APCI⁻): m/z 632.2 (M−H); Anal. Calcd for C₃₆H₃₇F₂N₂O₆Na₁.1.2H₂O: C, 63.93; H, 5.87; N, 4.14. Found: C, 64.00; H, 6.02; N, 3.82.

### Example 156

### (3R,5R)-7-[3-(4-Fluoro-phenyl)-1-isopropyl-5-methylcarbamoyl-4-phenyl-1H-pyrrol-2-yl]-3,5-dihydroxy-heptanoic Acid Sodium Salt

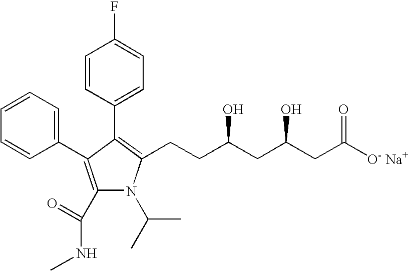

To a mixture of (3R,5R)-7-[3-(4-fluoro-phenyl)-1-isopropyl-5-methylcarbamoyl-4-phenyl-1H-pyrrol-2-yl]-3,5-dihydroxy-heptanoic acid methyl ester prepared in a similar manner to Example 125 step A-E (314 mg, 0.615 mmol) in a solution of absolute ethanol (2 mL) and water (0.5 mL) was added 1N sodium hydroxide solution (0.615 mL) at room temperature. The mixture was stirred for 1 h and then concentrated in vacuo to give a residue, which was dissolved in a solution of 20% methanol in methylene chloride and filtered. The filtrate was concentrated in vacuo to give a solid. The solid was triturated with diethyl ether and filtered and dried in vacuo to give 310 mg (97%) of the desired product as a white solid: mp 238-240° C.; MS(APCI⁻): m/z 495.2 (M−H); Anal. Calcd for C₂₈H₃₂F₁N₂O₅Na₁.1.7H₂O: C, 61.24; H, 6.50; N, 5.10. Found: C, 61.40; H, 6.37; N, 5.07.

### Example 157

### (3R,5S)-7-[3-(4-Fluoro-phenyl)-1-isopropyl-5-methylcarbamoyl-4-phenyl-1H-pyrrol-2-yl]-3,5-dihydroxy-hept-6-enoic Acid Sodium Salt

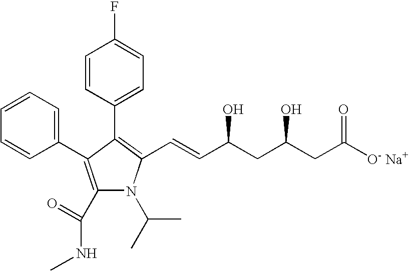

To a mixture of (3R,5S)-7-[3-(4-fluoro-phenyl)-1-isopropyl-5-methylcarbamoyl-4-phenyl-1H-pyrrol-2-yl]-3,5-dihydroxy-hept-6-enoic acid methyl ester prepared in a similar manner to Example 125 step A-D (200 mg, 0.393 mmol) in a solution of absolute ethanol (2 mL) and water (0.5 mL) was added 1N sodium hydroxide solution (0.393 mL) at room temperature. The mixture was stirred for 1 h and then concentrated in vacuo to give a residue, which was dissolved in a solution of 20% methanol in methylene chloride and filtered. The filtrate was concentrated in vacuo to give a solid. The solid was triturated with diethyl ether, filtered, and dried in vacuo to give 200 mg (98%) of the desired product as a white solid: mp 224-226° C.; MS(APCI⁻): m/z 494.1 (M−H); Anal. Calcd for C₂₈H₃₀F₁N₂O₅Na₁.1.7H₂O: C, 61.46; H, 6.15; N, 5.12. Found: C, 61.35; H, 5.89; N, 4.98.

### Example 158

### (3R,5R)-7-[5-Ethylcarbamoyl-3-(4-fluoro-phenyl)-1-isopropyl-4-phenyl-1H-pyrrol-2-yl]-3,5-dihydroxy-heptanoic Acid Sodium Salt

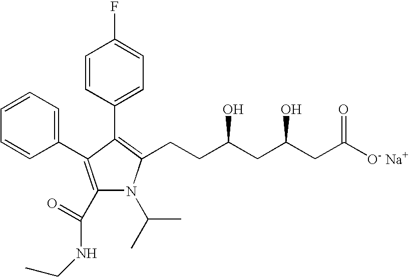

To a mixture of (3R,5R)-7-[5-ethylcarbamoyl-3-(4-fluoro-phenyl)-1-isopropyl-4-phenyl-1H-pyrrol-2-yl]-3,5-dihydroxy-heptanoic acid methyl ester prepared in a similar manner to Example 125 step A-E (251 mg, 0.478 mmol) in a solution of absolute ethanol (2 mL) and water (0.5 mL) was added 1N sodium hydroxide solution (0.478 mL) at room temperature. The mixture was stirred for 1 h and then concentrated in vacuo to give a residue, which was dissolved in a solution of 20% methanol in methylene chloride and filtered. The filtrate was concentrated in vacuo to give a solid. The solid was triturated with diethyl ether and filtered and dried in vacuo to give 250 mg (98%) of the desired product as a white solid: mp 203-205° C.; MS(APCI⁻): m/z 509.3 (M−H); Anal. Calcd for C₂₉H₃₄F₁N₂O₅Na₁.1.5H₂O: C, 62.24; H, 6.66; N, 5.01. Found: C, 62.28; H, 6.53; N, 4.85.

### Example 159

### (3R,5S)-7-[5-Ethylcarbamoyl-3-(4-fluoro-phenyl)-1-isopropyl-4-phenyl-1H-pyrrol-2-yl]-3,5-dihydroxy-hept-6-enoic Acid Sodium Salt

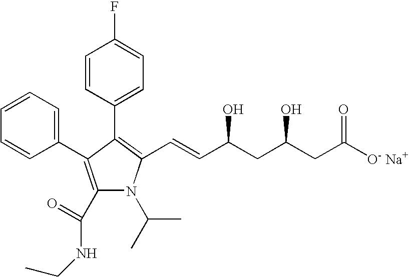

To a mixture of (3R,5S)-7-[5-ethylcarbamoyl-3-(4-fluoro-phenyl)-1-isopropyl-4-phenyl-1H-pyrrol-2-yl]-3,5-dihydroxy-hept-6-enoic acid methyl ester prepared in a similar manner to Example 125 step A-D (77.7 mg, 0.149 mmol) in a solution of absolute ethanol (1 mL) and water (0.5 mL) was added 1N sodium hydroxide solution (0.149 mL) at room temperature. The mixture was stirred for 1 h and then concentrated in vacuo to give a residue, which was dissolved in a solution of 20% methanol in methylene chloride and filtered. The filtrate was concentrated in vacuo to give a solid. The solid was triturated with diethyl ether and filtered and dried in vacuo to give 78.8 mg (100%) of the desired product as a white solid: mp 226-228° C.; MS(APCI⁻): m/z 508.2 (M−H); Anal. Calcd for C₂₉H₃₂F₁N₂O₅Na₁.1.3H₂O: C, 62.87; H, 6.30; N, 5.06. Found: C, 62.93; H, 6.36; N, 4.92.

### Example 160

### (3R,5S)-7-[5-Carbamoyl-3-(4-fluoro-phenyl)-1-isopropyl-4-phenyl-1H-pyrrol-2-yl]-3,5-dihydroxy-hept-6-enoic Acid Sodium Salt

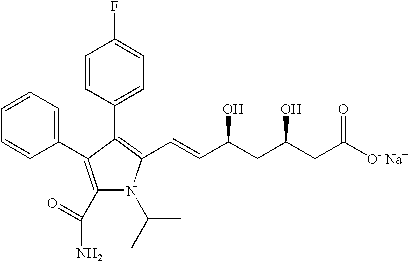

To a mixture of (3R,5S)-7-[5-carbamoyl-3-(4-fluoro-phenyl)-1-isopropyl-4-phenyl-1H-pyrrol-2-yl]-3,5-dihydroxy-hept-6-enoic acid methyl ester prepared in a similar manner to Example 125 step A-D (224 mg, 0.452 mmol) in a solution of absolute ethanol (1 mL) and water (0.5 mL) was added 1N sodium hydroxide solution (0.452 mL) at room temperature. The mixture was stirred for 1 h and then concentrated in vacuo to give a residue, which was dissolved in a solution of 20% methanol in methylene chloride and filtered. The filtrate was concentrated in vacuo to give a solid. The solid was triturated with diethyl ether and filtered and dried in vacuo to give 220 mg (97%) of the desired product as a white solid: mp 238-240° C.; MS(APCI⁻): m/z 479.2 (M−H); Anal. Calcd for C₂₇H₂₈F₁N₂O₅Na₁.1.3H₂O: C, 61.66; H, 5.86; N, 5.33. Found: C, 61.65; H, 5.94; N, 5.15.

### Example 161

### (3R,5R)-7-[5-Carbamoyl-3-(4-fluoro-phenyl)-1-isopropyl-4-phenyl-1H-pyrrol-2-yl]-3,5-dihydroxy-heptanoic Acid Sodium Salt

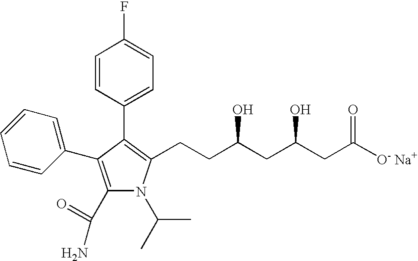 Step A

### 4-(4-Fluoro-phenyl)-5-formyl-1-isopropyl-3-phenyl-1H-pyrrole-2-carboxylic Acid Amide

A mixture of 4-(4-fluoro-phenyl)-5-formyl-1-isopropyl-3-phenyl-1H-pyrrole-2-carboxylic acid (Example 1, step G, 2.1 g, 5.69 mmol) in thionyl chloride (5 mL) was heated at reflux for 1 h. The resulting mixture was concentrated in vacuo to give a residue, which was dried in vacuo for 1 h. The crude acid chloride was dissolved in THF (10 mL) under a nitrogen atmosphere. The mixture was cooled in an ice bath and ammonium hydroxide (29.6% in water, 2.7 g, 22.7 mmol) was added dropwise. The mixture was stirred at room temperature overnight and partitioned between ethyl acetate and water. The organic phase was separated and washed with 1N HCl, NaHCO₃ and brine, dried over Na₂SO₄ and filtered. The filtrate was concentrated in vacuo to give a residue, which was purified by chromatography (10%-36% ethyl acetate in hexanes) to give 1.40 g (70%) of the desired product as a white solid: mp 187-188° C.; MS(APCI⁻): m/z 349.1 (M−H).

Step B

### 4-(4-Fluoro-phenyl)-5-hydroxymethyl-1-isopropyl-3-phenyl-1H-pyrrole-2-carboxylic Acid Amide

To a solution of 4-(4-fluoro-phenyl)-5-formyl-1-isopropyl-3-phenyl-1H-pyrrole-2-carboxylic acid amide (1.0 g, 2.85 mmol) in THF (10 mL) was added reducing reagent 1.0 M lithium tri-tert-butoxyaluminohydride in THF solution (3.42 mL, 3.42 mmol) dropwise in an ice bath under a nitrogen atmosphere. The mixture was stirred in an ice bath for 0.5 h at which point TLC was showed that the reaction was complete. The mixture was then partitioned between ethyl acetate and water. The organic phase was separated and washed with 1N HCl, NaHCO₃ and brine, dried over Na₂SO₄ and filtered. The filtrate was concentrated in vacuo to give a residue, which was purified by chromatography (10%-40% ethyl acetate in hexanes) to give 0.95 g (94%) of the desired product as a white solid: mp 189-190° C.; MS(APCI⁻): m/z 351.1 (M−H); Anal. Calcd for C₂₁H₂₁F₁N₂O₂: C, 71.57; H, 6.01; N, 7.95. Found: C, 71.24; H, 6.04; N, 7.75.

Step C

### [5-Carbamoyl-3-(4-fluoro-phenyl)-1-isopropyl-4-phenyl-1H-pyrrol-2-ylmethyl]-triphenyl-phosphonium Bromide

To a solution of 4-(4-fluoro-phenyl)-5-hydroxymethyl-1-isopropyl-3-phenyl-1H-pyrrole-2-carboxylic acid amide (909 mg, 2.58 mmol) in methylene chloride (40 mL) was added triphenylphosphine hydrobromide (885 mg, 2.58 mmol). The reaction was heated to 50° C. for 2.5 h after which time all starting material was consumed as determined by TLC. The mixture was then concentrated in vacuo to give 1.75 g (100%) white solid: mp 160-162° C.; MS(APCI⁺): m/z 597.0 (MH⁺); Anal. Calcd for C₃₉H₃₅Br₁F₁N₂O₁P₁.1.0H₂O: C, 67.04; H, 5.48; N, 3.78. Found: C, 67.34; H, 5.36; N, 4.03.

Step D

### Cis, trans-(4R,6R)-(6-{2-[5-Carbamoyl-3-(4-fluoro-phenyl)-1-isopropyl-4-phenyl-1H-pyrrol-2-yl]-vinyl}-2,2-dimethyl-[1,3]dioxan-4-yl)-acetic Acid Tert-Butyl Ester

To a solution of [5-carbamoyl-3-(4-fluoro-phenyl)-1-isopropyl-4-phenyl-1H-pyrrol-2-ylmethyl]-triphenyl-phosphonium bromide (435 mg, 0.642 mmol) in THF (15 mL) was added 1.0 M sodium bis(trimethylsilyl)amide in THF solution (0.83 mL, 0.83 mmol) dropwise at −78° C. under a nitrogen atmosphere. The reaction was stirred at −78° C. for 5 min after which time a solution of (6-formyl-2,2-dimethyl-[1,3]dioxan-4-yl)-acetic acid tert-butyl ester in 2 mL of THF (200 mg, 0.77 mmol) was added dropwise. The reaction was stirred at −78° C. for 30 min then allowed to warm to room temperature over 1.5 h. The mixture was then quenched with dropwise addition of saturated NH₄Cl. The organic phase was separated and washed with water and brine, dried over Na₂SO₄ and filtered. The filtrate was concentrated in vacuo to give a residue, which was purified by chromatography (10%-40% ethyl acetate in hexanes) to give 290 mg (78%) of the desired product as a white foam: mp 73-75° C.; MS(APCI⁻): m/z 575.3 (M−H); Anal. Calcd for C₃₄H₄₁F₁N₂O₅: C, 70.81; H, 7.17; N, 4.86. Found: C, 70.89; H, 7.25; N, 5.24.

Step E

### (4R,6R)-(6-{2-[5-Carbamoyl-3-(4-fluoro-phenyl)-1-isopropyl-4-phenyl-1H-pyrrol-2-yl]-ethyl}-2,2-dimethyl-[1,3]dioxan-4-yl)-acetic Acid Tert-Butyl Ester

To a solution of (4R,6R)-(6-{2-[5-carbamoyl-3-(4-fluoro-phenyl)-1-isopropyl-4-phenyl-1H-pyrrol-2-yl]-vinyl}-2,2-dimethyl-[1,3]dioxan-4-yl)-acetic acid tert-butyl ester(279 mg, 0.48 mmol) in THF (5 mL) and ethanol (10 mL) was added 10% palladium on activated carbon (50 mg). The mixture was stirred at room temperature under a hydrogen atmosphere for 3 h. TLC showed that the reaction was complete. The mixture was filtered through celite. The filtrate was concentrated in vacuo to give a residue, which was purified by chromatography (20-40% ethyl acetate in hexanes) to give 147 mg (53%) white solid: mp 201-202° C.; MS(APCI⁺): m/z 337.1 (M⁺); Anal. Calcd for C₂₁H₂₁F₁N₂O₅.0.5EtOAc: C, 72.61; H, 6.62; N. 7.36. Found: C, 72.47; H, 6.76; N, 6.99.

Step F

### (3R,5R)-7-[5-Carbamoyl-3-(4-fluoro-phenyl)-1-isopropyl-4-phenyl-1H-pyrrol-2-yl]-3,5-dihydroxy-heptanoic Acid Tert-Butyl Ester

To a mixture of (4R,6R)-(6-{2-[5-carbamoyl-3-(4-fluoro-phenyl)-1-isopropyl-4-phenyl-1H-pyrrol-2-yl]-ethyl)-2,2-dimethyl-[1,3]dioxan-4-yl)-acetic acid tert-butyl ester (128 mg, 0.22 mmol) in methanol (5 mL) was added 1N hyhrochloric acid (0.55 mL, 0.55 mmol) at room temperature. The mixture was stirred for 4 h at which time TLC was showed that the reaction was complete. The mixture was then partitioned between ethyl acetate and water. The organic phase was separated and washed with NaHCO₃ and brine, dried over Na₂SO₄ and filtered. The filtrate was concentrated in vacuo to give 119 mg (99%) of the desired product as a white solid: mp 94-96° C.; MS(APCI⁺): m/z 639.2 (MH⁺); Anal. Calcd for C₃₁H₃₉F₁N₂O₅.0.5H₂O: C, 67.99; H, 7.36; N, 5.12. Found: C, 67.79; H, 7.26; N, 5.04.

Step G

### (3R,5R)-7-[5-Carbamoyl-3-(4-fluoro-phenyl)-1-isopropyl-4-phenyl-1H-pyrrol-2-yl]-3,5-dihydroxy-heptanoic Acid Sodium Salt

To a mixture of (3R,5R)-7-[5-carbamoyl-3-(4-fluoro-phenyl)-1-isopropyl-4-phenyl-1H-pyrrol-2-yl]-3,5-dihydroxy-heptanoic acid tert-butyl ester (104 mg, 0.193 mmol) in a solution of absolute ethanol (1 mL) and water (0.5 mL) was added 1N sodium hydroxide solution (0.193 mL) at room temperature. The mixture was stirred for 1 h and then concentrated in vacuo to give a residue, which was triturated with methylene chloride and filtered and dried in vacuo to give 96 mg (98%) of the desired product as a white solid: mp 212-214° C.; MS(APCI⁻): m/z 481.2 (M−H); Anal. Calcd for C₂₇H₃₀F₁N₂O₅Na₁.1.35H₂O: C, 61.24; H, 6.29; N, 5.25. Found: C, 61.64; H, 6.37; N, 4.86.

### Example 162

### (3R,5R)-4-({[5-(6-Carboxy-3,5-dihydroxy-hexyl)-3,4-bis-(4-fluoro-phenyl)-1-isopropyl-1H-pyrrole-2-carbonyl]-amino}-methyl)-benzoic Acid Disodium Salt

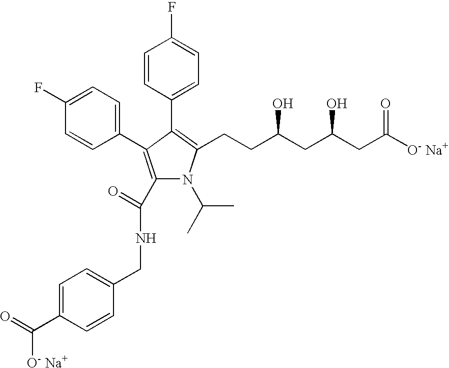 Step A

### (3R,5R)-4-({[5-(6-Carboxy-3,5-dihydroxy-hexyl)-3,4-bis-(4-fluoro-phenyl)-1-isopropyl-1H-pyrrole-2-carbonyl]-amino}-methyl)-benzoic Acid

To a mixture of (3R,5R)-4-({[5-(6-carboxy-3,5-dihydroxy-hexyl)-3,4-bis-(4-fluoro-phenyl)-1-isopropyl-1H-pyrrole-2-carbonyl]-amino)-methyl)-benzoic acid methyl ester (Example 129), 105 mg, 0.157 mmol) in a solution of methanol (10 mL) was added 1N sodium hydroxide solution (0.626 mL) at room temperature. The mixture was stirred at 60° C. for 2 h. The mixture was cooled down to room temperature and 1N hydrochloric acid (0.783 mL) was added. The mixture was concentrated in vacuo to give a residue, which was triturated with ethanol and filtered. The filtrate was concentrated in vacuo to give 98 mg (99%) of the desired product as a white solid: mp 140-142° C.; MS(APCI⁺): m/z 635.2 (MH⁺). The material was taken to the next step without further purification.

Step B

### (3R,5R)-4-({[5-(6-Carboxy-3,5-dihydroxy-hexyl)-3,4-bis-(4-fluoro-phenyl)-1-isopropyl-1H-pyrrole-2-carbonyl]-amino)-methyl)-benzoic Acid Disodium Salt

To a mixture of (3R,5R)-4-({[5-(6-carboxy-3,5-dihydroxy-hexyl)-3,4-bis-(4-fluoro-phenyl)-1-isopropyl-1H-pyrrole-2-carbonyl]-amino}-methyl)-benzoic acid (90.2 mg, 0.142 mmol) in a solution of absolute ethanol (1 mL) and water (0.5 mL) was added 1N sodium hydroxide solution (0.248 mL) at room temperature. The mixture was stirred for 1 h and then concentrated in vacuo to give a residue, which was. The solid was triturated with methylene chloride and filtered and dried in vacuo to give 95 mg (99%) of the desired product as a white solid: mp 298-300° C.; MS(APCI⁻): m/z 633.2 (M−H); Anal. Calcd for C₃₅H₃₄F₂N₂O₇Na₂.5.0H₂O.0.9CH₂Cl₂: C, 51.02; H, 5.46, N, 3.31. Found: C, 50.65; H, 5.20; N, 3.18.

### Example 163

### (3R,5R)-3-({[5-(6-Carboxy-3,5-dihydroxy-hexyl)-4-(4-fluoro-phenyl)-1-isopropyl-3-phenyl-1H-pyrrole-2-carbonyl]-amino}-methyl)-benzoic Acid Disodium Salt

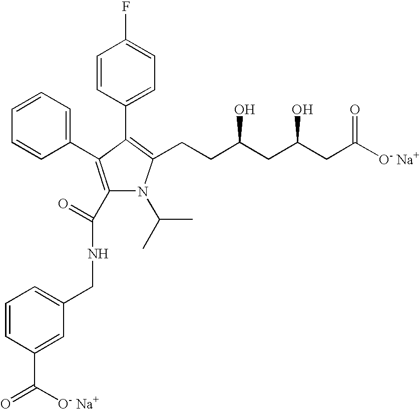 Step A

### (3R,5R)-3-({[5-(6-Carboxy-3,5-dihydroxy-hexyl)-4-(4-fluoro-phenyl)-1-isopropyl-3-phenyl-1H-pyrrole-2-carbonyl]-amino}-methyl)-benzoic Acid

To a mixture of (3R,5R)-3-({[5-(6-carboxy-3,5-dihydroxy-hexyl)-4-(4-fluoro-phenyl)-1-isopropyl-3-phenyl-1H-pyrrole-2-carbonyl]-amino}-methyl)-benzoic acid methyl ester (Example 150), 385 mg, 0.590 mmol) in a solution of methanol (10 mL) was added 1N sodium hydroxide solution (2.36 mL) at room temperature. The mixture was stirred at 60° C. for 2 h. The mixture was cooled down to room temperature and 1N hydrochloric acid (2.95 mL) was added. The mixture was concentrated in vacuo to give a residue, which was triturated with 1:1 ethanol-methylene chloride and filtered. The filtrate was concentrated in vacuo to give 360 mg (99%) of the desired product as a white solid: mp 140-141° C.; MS(APCI⁺): m/z 617.1 (MH⁺). The material was taken to the next step without further purification.

Step B

### (3R,5R)-3-({[5-(6-Carboxy-3,5-dihydroxy-hexyl)-4-(4-fluoro-phenyl)-1-isopropyl-3-phenyl-1H-pyrrole-2-carbonyl]-amino}-methyl)-benzoic Acid Disodium Salt

To a mixture of (3R,5R)-3-({[5-(6-carboxy-3,5-dihydroxy-hexyl)-4-(4-fluoro-phenyl)-1-isopropyl-3-phenyl-1H-pyrrole-2-carbonyl]-amino}-methyl)-benzoic acid (80.9 mg, 0.131 mmol) in a solution of absolute ethanol (1 mL) and water (0.5 mL) was added 1N sodium hydroxide solution (0.262 mL) at room temperature. The mixture was stirred for 1 h and then concentrated in vacuo to give a residue, which was. The solid was triturated with methylene chloride and filtered and dried in vacuo to give 86 mg (99%) of the desired product as a white solid: mp 240-245° C.; MS(APCI⁻): m/z 615.2 (M−H); Anal. Calcd for C₃₅H₃₅F₁N₂O₇Na₂.2.4H₂O: C, 57.37; H, 5.92; N, 3.82. Found: C, 57.35; H, 5.54; N, 3.53.

### Example 164

### (3R,5R)-4-({[5-(6-Carboxy-3,5-dihydroxy-hexyl)-4-(4-fluoro-phenyl)-1-isopropyl-3-phenyl-1H-pyrrole-2-carbonyl]-amino}-methyl)-benzoic Acid Disodium Salt

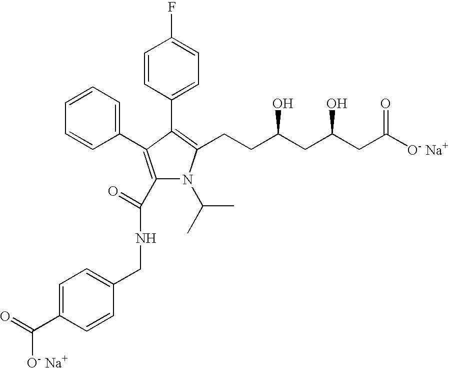 Step A

### (3R,5R)-4-({[5-(6-Carboxy-3,5-dihydroxy-hexyl)-4-(4-fluoro-phenyl)-1-isopropyl-3-phenyl-1H-pyrrole-2-carbonyl]-amino-methyl)-benzoic Acid

To a mixture of (3R,5R)-4-({[5-(6-carboxy-3,5-dihydroxy-hexyl)-4-(4-fluoro-phenyl)-1-isopropyl-3-phenyl-1H-pyrrole-2-carbonyl]-amino}-methyl)-benzoic acid isopropyl ester (Example 148), 317 mg, 0.466 mmol) in a solution of methanol (10 mL) was added 1N sodium hydroxide solution (1.86 mL) at room temperature. The mixture was stirred at 60° C. for 2 h. The mixture was cooled down to room temperature and 1N hydrochloric acid (2.33 mL) was added. The mixture was concentrated in vacuo to give a residue, which was triturated with ethanol and filtered. The filtrate was concentrated in vacuo to give 290 mg (99%) of the desired product as a white solid: mp 93-95° C.; MS(APCI⁺): m/z 631.2 (MH⁺). The material was taken to the next step without further purification.

Step B

### (3R,5R)-4-({[5-(6-Carboxy-3,5-dihydroxy-hexyl)-4-(4-fluoro-phenyl)-1-isopropyl-3-phenyl-1H-pyrrole-2-carbonyl]-amino}-methyl)-benzoic Acid Disodium Salt

To a mixture of (3R,5R)-4-({[5-(6-carboxy-3,5-dihydroxy-hexyl)-4-(4-fluoro-phenyl)-1-isopropyl-3-phenyl-1H-pyrrole-2-carbonyl]-amino}-methyl)-benzoic acid (79.5 mg, 0.126 mmol) in a solution of absolute ethanol (1 mL) and water (0.5 mL) was added 1N sodium hydroxide solution (0.252 mL) at room temperature. The mixture was stirred for 1 h and then concentrated in vacuo to give a residue, which was. The solid was triturated with methylene chloride and filtered and dried in vacuo to give 83 mg (100%) of the desired product as a white solid: mp 255-260° C.; MS(APCI⁺): m/z 617.1 (MH⁺); Anal. Calcd for C₃₅H₃₅F₁N₂O₇Na₂.3.30H₂O: C, 56.39; H, 5.67; N, 3.72. Found: C, 55.99; H, 5.31; N, 3.56.

### Example 165

### Sodium; (3R,5R)-7-[3,4-bis-(4-fluoro-phenyl)-5-(4-fluoro-phenylcarbamoyl)-1-isopropyl-1H-pyrrol-2-yl]-3,5-dihydroxy-heptanoate

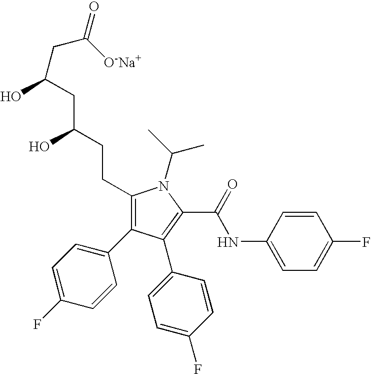 Step A

### 3,4-Bis(4-fluorophenyl)-5-formyl-1-isopropyl-1H-pyrrole-2-carboxylic Acid (4-fluorophenyl)-amide

To a solution of 3,4-bis(4-fluorophenyl)-5-formyl-1-isopropyl-1H-pyrrole-2-carboxylic Acid (0.50 g, 1.4 mmol) and 1-3 drops DMF in dry THF(10 mL) chilled in an ice-bath under a nitrogen atmosphere was added oxalyl chloride (0.11 mL, 1.4 mmol). The resulting mixture was stirred 1 h, warmed to room temperature, and stirred 3h. After stirring, 4-fluoroaniline(0.30 g, 2.7 mmol) was added followed by triethylamine (0.19 mL, 1.4 mmol). The reaction mixture was stirred at room temperature overnight and partitioned between ethyl acetate and water. The organic phase was separated and washed with 1N HCl, NaHCO₃ and brine, dried over Na₂SO₄ and filtered. The filtrate was concentrated in vacuo to give a residue, which was purified by recrystallization in methanol and water to give 0.41 g (66%) of the desired product as a white solid: MS(APCI⁺): m/z 463.2 (M+H); NMR (CDCl₃) δ 1.65 (6H, d, J=6.8 Hz), 4.80 (1H, septet, J=7.0 Hz), 6.90-7.00 (6H, m), 7.02-7.10 (6H, m), 9.50 (1H, s).

Step B

### (3R)-7-[3,4-Bis(4-fluorophenyl)-5-(4-fluorophenylcarbamoyl)-1-isopropyl-1H-pyrrol-2-yl]-3-(tert-butyl-dimethyl-silanyloxy)-5-oxo-hept-6-enoic Acid Methyl Ester

To a mixture of 3,4-Bis(4-fluorophenyl)-5-formyl-1-isopropyl-1H-pyrrole-2-carboxylic acid (2-fluorophenyl)amide (0.38 g, 0.82 mmol) from Step A in toluene (30 mL), at room temperature, under a nitrogen atmosphere, was added wittig reagent [(3R)-3-(tert-butyl-dimethyl-silanyloxy)-5-oxo-6-(triphenyl-phosphanylidene)-hexanoic acid methyl ester] (0.88 g, 1.6 mmol). The mixture was heated at reflux for 64 h and then concentrated in vacuo to give a residue, which was purified by chromatography (1%-50% EtOAc in Hexane) to give 0.38 g (65%) of the desired product as an yellow foam: MS(APCI+): m/z 719.2 (M+1); NMR (CDCl3) δ−0.37 (6H, d, J=20 Hz), 0.77 (9H, s), 1.65 (6H, d, J=7.3 Hz), 2.39-2.58 (4H, m), 3.62 (3H, s), 4.46 (1H, septet, J=7.0 Hz), 5.23-5.28 (1H, m), 5.90 (1H, d, J=16), 6.83-7.10 (12H, m), 7.69 (1H, d, J=16 Hz).

Step C

### (3R)-7-[3,4-Bis(4-fluorophenyl)-5-(4-fluorophenylcarbamoyl)-1-isopropyl-1H-pyrrol-2-yl]-3-hydroxy-5-oxo-hept-6-enoic Acid Methyl Ester

To a solution of (3R)-7-[3,4-Bis(4-fluorophenyl)-5-(4-fluorophenylcarbamoyl)-1-isopropyl-1H-pyrrol-2-yl]-3-(tert-butyl:dimethyl-silanyloxy)-5-oxo-hept-6-enoic acid methyl ester from Step B (0.13 g, 0.17 mmol) in acetonitrile (5 mL) was added dropwise a hydrogen fluoride solution (1:10 48% HF:acetonitrile, 1.0 mL), in an ice bath, under a nitrogen atmosphere. The mixture was stirred at room temperature for 3 h. TLC showed that the reaction was complete. The mixture was diluted with saturated aqueous NaHCO3, partitioned between ethyl acetate and water. The organic phase was separated and washed with brine, dried over Na₂SO₄ and filtered. The filtrate was concentrated in vacuo and used as is in the subsequent reaction.

Step D

### (3R,5S)-7-[3,4-Bis(4-fluorophenyl)-5-(4-fluorophenylcarbamoyl)-1-isopropyl-1H-pyrrol-2-yl]-3,5-dihydroxy-hept-6-enoic Acid Methyl Ester

To a mixture of (3R)-7-[3,4-Bis(4-fluorophenyl)-5-(4-flourophenylcarbamoyl)-1-isopropyl-1H-pyrrol-2-yl]-3-hydroxy-5-oxo-hept-6-enoic acid methyl ester from Step C (0.10 g, 0.17 mmol), in THF (10 mL), was added dropwise a solution of 0.5M diethyl-methoxy-silane in THF (0.85 mL) at −78° C. under a nitrogen atmosphere. The mixture was stirred for 0.5 h and then sodium borohydride (13 mg, 0.33 mmol) was added in portions. After stirring for 2 h, a few drops of acetic acid were added and the mixture was partitioned between ethyl acetate and water. The organic phase was separated and washed with NaHCO₃ and brine, dried over Na₂SO₄ and filtered. The filtrate was concentrated in vacuo to give a residue, which was dissolved in warm methanol and concentrated in vacuo again to give a residue, which was purified by preparative TLC chromatography (50% ethyl acetate in hexanes) to give 83 mg (84%) of the desired product as a white foam; MS(APCI⁺): m/z 607.2 (M+H); NMR (DMSO) δ 1.29-1.34 (1H, m), 1.39-1.43, (1H, m), 1.51 (6H, d, J=6.6 Hz), 2.38-2.41 (2H, m), 3.57 (1H, s), 3.66 (3H, s), 3.76 (1H, s), 4.05-4.14 (1H, m), 4.32-4.35 (1H, m), 5.19 (1H, septet, J=6.6), 5.30 (2H, d, J=14 Hz), 6.70 (1H, d, J+14 Hz) 6.81-7.05 (12H, m).

Step E

### (3R,5R)-7-[3,4-bis-(4-fluorophenyl)-5-(4-fluorophenylcarbamoyl)-1-isopropyl-1H-pyrrol-2-yl]-3,5-dihydroxy-heptanoic Acid Methyl Ester

To a solution of (3R,5S)-7-[3,4-Bis-(4-fluoro-phenyl)-1-isopropyl-5-(4-fluorophenylcarbonyl)-1H-pyrrol-2-yl]-3,5-dihydroxy-hept-6-enoic acid methyl ester (0.52 g, 0.86 mmol) in THF (10 mL) was added 10% Palladium on activated carbon (0.45 g). This mixture was stirred at room temperature under hydrogen atmosphere for 3 h then filtered through celite. The filtrate was concentrated in vacuo to give a residue which was purified by flash chromatography (10%-100% EtOAc/Hexane) to give 290 mg (56%) of a white solid: MS(APCI−): m/z 609.1 (M+H); NMR (CDCl3) δ 1.29-1.34 (1H, m), 1.35-1.49, (6H, m), 1.51 (6H, dd, J=7.1 Hz, J=1Hz), 2.36-2.39 (2H, m), 2.64-2.70 (1H, m), 2.80-2.85 (1H, m), 3.67 (3H, s), 3.76 (1H, s), 3.71-3.76 (1H, m), 4.08-4.14 (1H, m), 4.32-4.35 (1H, m), 5.19 (1H, sept, J=7.1), 6.82-7.05 (12H, m).

Step F

### Sodium; (3R,5R)-7-[3,4-bis-(4-fluoro-phenyl)-5-(4-fluoro-phenylcarbamoyl)-1-isopropyl-1H-pyrrol-2-yl]-3,5-dihydroxy-heptanoate

To a solution of (3R,5R)-7[3,4-bis(fluorophenyl)-5-(4-fluorophenylcarbamoyl)-1-isopropyl-1-H-pyrrol-2-yl]-3,5-dihydroxy-heptanoic acid methyl ester from Step E (0.26g, 0.43 mmol) in a solution of absolute ethanol (5.0 mL) was added 1.0N aqueous sodium hydroxide solution (0.50 mL) at room temperature. The mixture was stirred for 1 h and then concentrated in vacuo to give a residue, which was dissolved in a solution of 20% methanol in methylene chloride and filtered. The filtrate was concentrated in vacuo to give a solid. The solid was triturated with dichloromethane, filtered, and dried in vacuo to give 223 mg (100%) of the desired product as a white solid: MS(APCI⁺): m/z 595.1 (M+1); Anal. Calcd for C₃₂H₃₂F₃N₂O₅.1.01H₂O: C, 62.44; H, 5.40; N, 4.41. Found: C, 62.05; H, 5.13; N. 4.24.

### Example 166

### Sodium;(3R,5R)-7-[3,4-bis(4-fluoro-phenyl)-5-(3-fluoro-phenylcarbamoyl)-1-isopropyl-1-H-pyrrol-2-yl]-3,5-dihydroxy-heptanoate

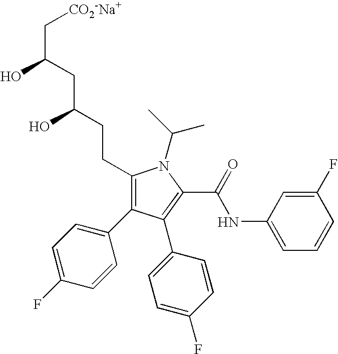

Synthesized in a similar manner to Example 165. MS(APCI⁺): m/z 595.1 (M+1); Anal. Calcd for C₃₃H₃₂F₃N₂O₅Na.0.90H₂O: C, 62.63; H, 5.38; N, 4.43. Found: C, 62.24; H, 5.23; N, 4.22.

### Example 167

### Sodium;(3R,5R)-7-[5-(3,5-difluoro-phenylcarbamoyl)-3-(4-fluoro-phenyl)-1-isopropyl-4-phenyl-1H-pyrrol-2-yl]-3,5-dihydroxy-heptanoate

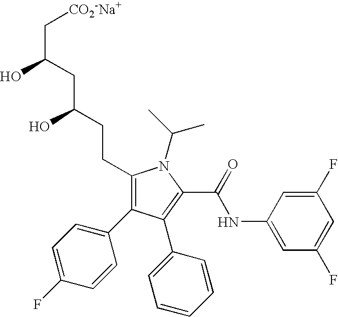

Synthesized in a similar manner to Example 165. MS(APCI⁺): m/z 595.0 (M+1); Anal. Calcd for C₃₃H₃₂F₃N₂O₅Na.1.45H2O: C, 61.67; H, 5.47; N, 4.36. Found: C, 61.28; H, 5.07; N, 4.20.

### Example 168

### Sodium;(3R,5R)-7-[5-(4-ethoxycarbonyl-benzylcarbamoyl)-3-(4-fluoro-phenyl)-1-isopropyl-4-phenyl-1H-pyrrol-2-yl]-3,5-dihydroxy-heptanoate

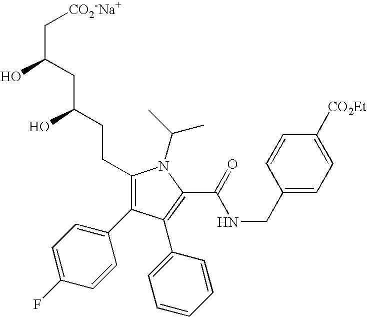

Synthesized in a similar manner to Example 165. MS(APCI⁺): m/z 645.2 (M+1); Anal. Calcd for C₃₇H₄₀FN₂O₇Na₁.1.35H2O: C, 64.31; H, 6.23; N, 4.05. Found: C, 63.92; H, 6.05; N, 4.92.

### Example 169

### Sodium; trans-(3R,5 S)-7-[5-(3-ethoxycarbonyl-benzylcarbamoyl)-3-(4-fluoro-phenyl)-1-isopropyl-4-phenyl-1H-pyrrol-2-yl]-3,5-dihydroxy-hept-6-enoate

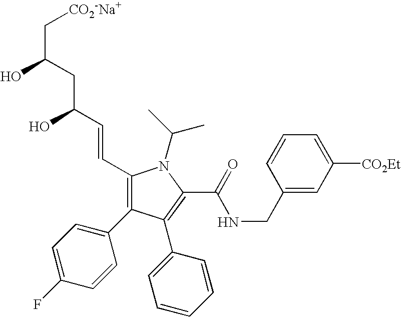 Step A
Intermediate 1

### Trans-(3S,5R)-3-({[5-(3,5-dihydroxy-6-methoxycarbonyl-hex-1-enyl)-4-(4-fluorophenyl)-1-isopropyl-3-phenyl-1H-pyrrole-carbamyl]-amino)-methyl)-benzoic Acid Methyl Ester

Synthesized in a similar manner to Example 165 Steps A through D. MS(APCI⁺): m/z 657.2 (M+1); Anal. Calcd for C₃₇H₃₈FN₂O₇.0.11C₄H₈O₂: C, 69.28; H, 6.33; N, 4.20. Found: C, 68.89; H, 6.33; N, 4.24.

Step B

### Sodium; trans-(3R,5 S)-7-[5-(3-ethoxycarbonyl-benzylcarbamoyl)-3-(4-fluoro-phenyl)-1-isopropyl-4-phenyl-1H-pyrrol-2-yl]-3,5-dihydroxy-hept-6-enoate

To a solution of(3S,5R)-3-({[5-(3,5-dihydroxy-6-methoxycarbonyl-hex-1-enyl)-4-(4-fluorophenyl)-1-isopropyl-3-phenyl-1H-pyrrole-carbamyl]-amino}-methyl)-benzoic acid methyl ester, Intermediate 1, (120 mg, 0.182 mmol) in a solution of absolute ethanol (5.0 mL) was added 1.0N aqueous sodium hydroxide solution (0.19 mL) at room temperature. The mixture was stirred for 1 h and then concentrated in vacuo to give a residue, which was dissolved in a solution of 20% methanol in methylene chloride and filtered. The filtrate was concentrated in vacuo to give a solid. The solid was triturated with dichloromethane, filtered, and dried in vacuo to give 113 mg (93%) of the desired product as a white solid: MS(APCI⁺): m/z 643.2 (M+1); Anal. Calcd for C₃₇H₃₈FN₂O₇Na.1.45H₂O: C, 64.33; H, 5.97; N, 4.06. Found: C, 63.94; H, 5.57; N, 4.06.

### Example 170

### Sodium;(3R,5R)-7-[5-(3-ethoxycarbonyl-benzylcarbamoyl)-3-(4-fluoro-phenyl)-1-isopropyl-4-phenyl-1H-pyrrol-2-yl]-3,5-dihydroxy-heptanoate

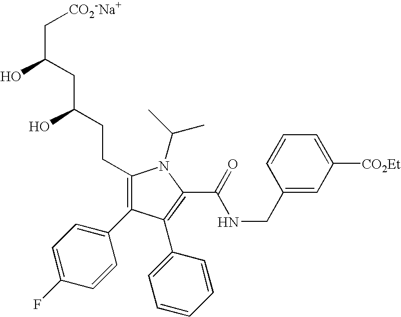

Synthesized in a similar manner to Example 165. MS(APCI⁺): m/z 645.2 (M+1); Anal. Calcd for C₃₇H₄₀FN₂O₇Na.1.05H₂O: C, 64.82; H, 6.19; N, 4.09. Found: C, 64.82; H, 5.79; N, 4.03.

### Example 171

### Sodium; (3R,5S)-7-[5-(2,3-dimethoxy-benzylcarbonyl)-3-(4-fluoro-phenyl)-1-isopropyl-4-phenyl-1H-pyrrol-2-yl]-3,5-dihydroxy-hept-6-enoate

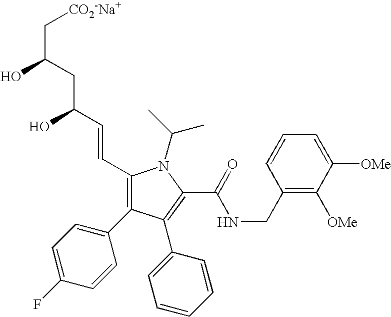 Step A
Intermediate 2

### (3R,5S)-7-{5-(2,3-dimethoxy-benzylcarbamoyl)-3-(4-fluoro-phenyl-1-isopropyl-4-phenyl-1H-pyrrol-2-yl]-3,5-dihydroxy-hept-6-enoic Acid Methyl Ester

Synthesized in a similar manner to Example 1SM, Steps A through D. MS(APCI⁺): m/z 647.3 (M+1); Anal. Calcd for C₃₇H₄₃FN₂O₇.0.25C₄H₈O₂: C, 68.25; H, 6.70; N, 4.23. Found: C, 67.86; H, 6.70; N, 4.23.

Step B

### Sodium;(3R,5S)-7-[5-(2,3-dimethoxy-benzylcarbonyl)-3-(4-fluoro-phenyl)-1-isopropyl-4-phenyl-1H-pyrrol-2-yl]-3,5-dihydroxy-hept-6-enoate

To a solution of (3R,5S)-7-{5-(2,3-dimethoxy-benzylcarbamoyl)-3-(4-fluoro-phenyl-1-isopropyl-4-phenyl-1H-pyrrol-2-yl]-3,5-dihydroxy-hept-6-enoic acid methyl ester, Intermediate 2, (101 mg, 0.155 mmol) in a solution of absolute ethanol (5.0 mL) was added 0.1N aqueous sodium hydroxide solution (1.6 mL) at room temperature. The mixture was stirred for 1 h and then concentrated in vacuo to give a residue, which was dissolved in a solution of 20% methanol in methylene chloride and filtered. The filtrate was concentrated in vacuo to give a solid. The solid was triturated with ether, filtered, and dried in vacuo to give 70 mg (69%) of the desired product as a white solid: MS(APCI⁺): m/z 631.2 (M+1); Anal. Calcd for C₃₆H₃₈FN₂O₇Na.2.34H₂O: C, 62.23; H, 6.19; N, 4.03. Found: C, 61.83; H, 5.71; N, 3.94.

### Example 172

### Sodium;(3R,5R)-7-[5-(2,3-dimethoxy-benzylcarbonyl)-3-(4-fluoro-phenyl)-1-isopropyl-4-phenyl-1H-pyrrol-2-yl]-3,5-dihydroxy-heptanoate

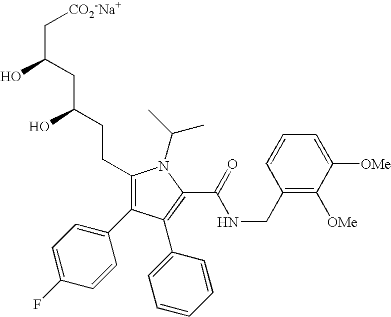

Synthesized in a similar manner to Example 165. MS(APCI⁺): m/z 633.2 (M+1); Anal. Calcd for C₃₆H₄₀FN₂O₇Na.2.37H2O: C, 62.00; H, 6.47; N, 4.02. Found: C, 61.60; H, 6.40; N, 3.85.

### Example 173

### Sodium;(3R,5R)-7-[3-(4-fluoro-phenyl)-1-isopropyl-5-[(5-methoxy-pyridin-2ylmethyl)-carbamoyl]-4-phenyl-1H-pyrrol-2-yl]-3,5-dihydroxy-heptanoate

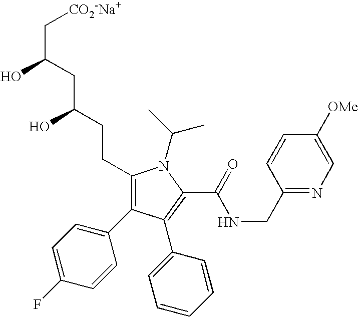

Synthesized in a similar manner to Example 165. MS(APCI⁺): m/z 604.2 (M+1); Anal. Calcd for C₃₄H₄₀FN₂O₇Na.4.35H₂O-0.55CH₂Cl₂: C, 55.27; H, 6.28; N, 5.60. Found: C, 55.57; H, 5.92; N, 5.20.

### Example 174

### (3R,5R)-7-[3-(4-Fluoro-phenyl)-5-[(4-hydroxy-phenylcarbamoyl)-1-isopropyl-4-phenyl-1H-pyrrol-2-yl]-3,5-dihydroxy-heptanoic Acid Disodium Salt

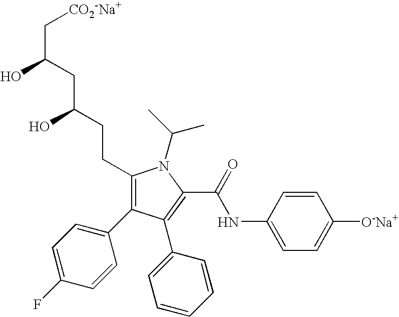 Step A
Intermediate 3

### (3R,5S)-7-[5-(4-Benzyloxy-phenylcarbamoyl)-3-(4-fluoro-phenyl)-1-isopropyl-4-phenyl-1H-pyrrol-2-yl]-3,5-dihydroxy-hept-6-enoic Acid Methyl Ester

Synthesized in a similar manner to Example 1, Steps A through D. MS(APCI⁺): m/z 677.0 (M+1); Anal. Calcd for C₄₁H₄₁FN₂O₆: C, 72.76; H, 6.11; N, 4.14. Found: C, 72.37; H, 6.01; N, 4.02.

Step B

Intermediate 4

### (3R,5R)-7-[3-(4-Fluoro-phenyl)-5-[(4-hydroxy-phenylcarbamoyl)-1-isopropyl-4-phenyl-1H-pyrrol-2-yl]-3,5-dihydroxy-heptanoic Methyl Ester

To a solution of (3R,5S)-7-[5-(4-Benzyloxy-phenylcarbamoyl)-3-(4-fluoro-phenyl)-1-isopropyl-4-phenyl-1H-pyrrol-2-yl]-3,5-dihydroxy-hept-6-enoic acid methyl ester, Intermediate 3, (0.70 g, 1.0 mmol) in THF (10 mL) was added 10% Palladium on activated carbon (0.45 g). This mixture was stirred at room temperature under hydrogen atmosphere for 3 h then filtered through celite. The filtrate was concentrated in vacuo to give a residue which was purified by flash chromatography (15%-95% EtOAc/Hexane) to give 287 mg (47%) of a white solid: MS(APCI⁺): m/z 589.0 (M+1); Anal. Calcd for C₃₄H₃₇FN₂O₆: C, 69.37; H, 6.34; N, 4.76. Found: C, 69.28; H, 6.24; N, 4.64.

Step C

### (3R,5R)-7-[3-(4-Fluoro-phenyl)-5-[(4-hydroxy-phenylcarbamoyl)-1-isopropyl-4-phenyl-1H-pyrrol-2-yl]-3,5-dihydroxy-heptanoic Acid Disodium Salt

To a solution of (3R,5R)-7-[3-(4-fluoro-phenyl)-5-[(4-hydroxy-phenylcarbamoyl)-1-isopropyl-4-phenyl-1H-pyrrol-2-yl]-3,5-dihydroxy-heptanoic methyl ester, Intermediate 4, (240 mg, 0.407 mmol) in a solution of absolute ethanol (5.0 mL) was added 0.1N aqueous sodium hydroxide solution (0.30 mL) at room temperature. The mixture was stirred for 1 h and then concentrated in vacuo to give a residue, which was dissolved in a solution of 20% methanol in methylene chloride and filtered. The filtrate was concentrated in vacuo to give a solid. The solid was triturated with ether, filtered, and dried in vacuo to give 122 mg (48%) of the desired product as a white solid: MS(APCI⁺): m/z 575.0 (M+1); Anal. Calcd for C₃₃H₃₃FN₂O₆Na₂.1.95H2O: C, 60.63; H, 5.69; N, 4.29. Found: C, 60.24; H, 5.51; N, 4.00.

### Example 175

### Trans-(3S,5R)-4-({[5-(6-Carboxy-3,5-dihydroxy-6-hex-1-enyl)-4-(4-fluorophenyl)-1-isopropyl-3-phenyl-1H-pyrrole-carbonyl]-amino}-methyl)-benzoic Acid Disodium Salt

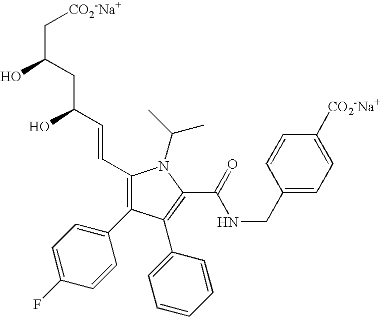 Step A
Intermediate 5

### Trans-(3S,5R)-4-({[5-(3,5-Dihydroxy-6-methoxycarbonyl-hex-1-enyl)-4-(4-fluorophenyl)-1-isopropyl-3-phenyl-1H-pyrrole-carbonyl]-amino}-methyl)-benzoic Acid Benzyl Ester

Synthesized in a similar manner to Example 1, Steps A through D. MS(APCI⁺): m/z 719.2 (M+1); Anal. Calcd for C₄₃H₄₃FN₂O₇: C, 71.85; H. 6.03; N, 3.90. Found: C, 71.68; H, 6.09; N, 3.83.

Step B

### Trans-(3S,5R)-4-({[5-(6-Carboxy-3,5-dihydroxy-6-hex-1-enyl)-4-(4-fluorophenyl)-1-isopropyl-3-phenyl-1H-pyrrole-carbonyl]-amino}-methyl)-benzoic Acid Disodium Salt

To a solution of Trans-(3S,5R)-4-({[5-(3,5-Dihydroxy-6-methoxycarbonyl-hex-1-enyl)-4-(4-fluorophenyl)-1-isopropyl-3-phenyl-1H-pyrrole-carbonyl]-amino}-methyl)-benzoic acid benzyl ester, Intermediate 5, (120 mg, 0.167 mmol) in a solution of absolute ethanol (5.0 mL) was added 0.1N aqueous sodium hydroxide solution (0.19 mL) at room temperature. The mixture was stirred for 1 h and then concentrated in vacuo to give a residue, which was dissolved in a solution of 20% methanol in methylene chloride and filtered. The filtrate was concentrated in vacuo to give a solid. The solid was triturated with ether, filtered, and dried in vacuo to give 105 mg (96%) of the desired product as a white solid: MS(APCI⁺): m/z 629.2 (M+1); Anal. Calcd for C₃₅H₃₃FN₂O₆Na₂.4.05H₂O: C, 57.46; H, 5.66; N, 3.58. Found: C, 57.07; H, 5.31; N. 3.58.

### Example 176

### Sodium;(3R,5R)-7-[5-dimethylcarbamoylcarboyl-3-(4-fluoro-phenyl)-1-isopropyl-4-phenyl-1H-pyrrol-2-yl]-3,5-dihydroxy-heptanoate

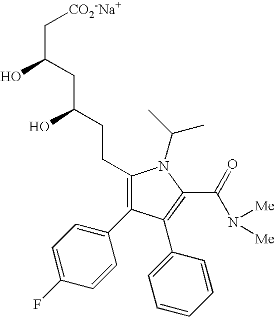 Step A
Intermediate 6

### 4-(4-Fluoro-phenyl)-5-formyl-1-isopropyl-3-phenyl-1H-pyrrole-2-carboxylic Acid Dimethylamide

Synthesized in a similar manner to Example 165 Step A. MS(APCI⁺): m/z.379.2 (M+1); Anal. Calcd for C₂₃H₂₃FN₂O₂: C, 73.00; H, 6.13; N, 7.40. Found: C, 72.79; H, 6.14; N, 7.26.

Step B

Intermediate 7

### 4-(4-Fluoro-phenyl)-5-hydroxymethyl-1-isopropyl-3-phenyl-1H-pyrrole-2-carboxylic Acid Dimethylamide

To a solution of 4-(4-Fluoro-phenyl)-5-formyl-1-isopropyl-3-phenyl-1H-pyrrole-2-carboxylic acid dimethylamide, Intermediate 6, (1.5 g, 4.0 mmol) in THF:MeOH (1:1, 30 mL) at −10° C. was added NaBH4 (0.18g, 4.89 mmol). The reaction mixture was stirred at 10° C. for 0.5 h, then the solvent was removed under vacuum. The residue was dissolved in DMC, washed with 5% NaHCO₃, dried over Na₂SO₄, and concentrated under vacuum. The crude product was purified by flash chromatography to give 1.14 g (76%) of white solid. MS(APCI⁺): m/z.381.1 (M+1); Anal. Calcd for C₂₃H₂₅FN₂O₂.0.05C₄H₈O₂: C, 72.40; H, 6.68; N, 7.28. Found: C, 72.02; H, 6.65; N, 7.09.

Step C

Intermediate 8

### 5-Demethyllcarbmoyl-3-(4-fluoro-phenyl)-1-isopropyl-3-phenyl-1H-pyrrol-2-yl-phosphonium; Bromide

To a solution of 4-(4-Fluoro-phenyl)-5-hydroxymethyl-1-isopropyl-3-phenyl-1H-pyrrole-2-carboxylic acid dimethylamide (1.1 g, 2.9 mmol) in DCM (10 mL) was added triphenylphosphine hydrobromide (1.0 g, 2.9 mmol) under nitrogen. The resulting mixture was stirred 2.5 h, concentrated, and used is after drying under vacuum at room temperature for 16 h.

Step D

Intermediate 9

### Cis, trans-(4R,6S)-(6-{2-[5-dimethylcarbamoyl-3-94-fluoro-phenyl)-4-phenyl-1H-pyrrol-2-yl]-vinyl}-2,2-dimethyl-[1,3]dioxin-4-yl-acetic Acid Tert-Butyl Ester

To a solution of 5-demethyllcarbmoyl-3-(4-fluoro-phenyl)-1-isopropyl-3-phenyl-1H-pyrrol-2-yl-phosphonium bromide, Intermediate 8, (2.0 g, 1.2 mmol) in THF(25 mL) at −78° C. under nitrogen was added 1.0 M NaHMDS in THF (3.7 mL). The resulting mixture was stirred 5 min at −78° C., during which time a orange color was noted, after which a solution of (6-fromyl-2,2-dimethyl-[1,3]dioxin-4-yl)-acetic acid tert-butyl ester (0.88 g) in THF (5 mL) was added dropwise. The reaction mixture was stirred at −78° C. for 30 min then allowed to warm to room temperature over 1.5h. The reaction mixture was concentrated under vacuum and the residue dissolved in EtOAc. The organic phase was washed with water and brine then dried over Na2SO4 and concentrated under vacuum.

The residue was purified by flash chromatography (0 to 100% EtOAc/Hexane) to give 1.41 g of a waxy yellow solid. NMR showed a 6:1 mixture of 4-(4-fluro-phenyl)-1-isopropyl-5-methyl-3-phenyl-1H-pyrrole-2-carboxylic acid dimethylamide and Cis, trans-(4R,6S)-(6-{2-[5-dimethylcarbamoyl-3-94-fluoro-phenyl)-4-phenyl-1H-pyrrol-2-yl]-vinyl}-2,2-dimethyl-[1,3]dioxin-4-yl-acetic acid tert-butyl ester. Used as is.

Step E

Intermediate 10

### (3R,5R)-7-[5-Dimethylcarbamoyl-3-(4-fluoro-phenyl)-1-isopropyl-4-phenyl-1H-pyrrol-2-yl]-3,5-dihydroxy-heptanoic Acid Tert-Butyl Ester

The mixture of 4-(4-Fluro-phenyl)-1-isopropyl-5-methyl-3-phenyl-1H-pyrrole-2-carboxylic acid dimethyl amide and Cis,trans-(6-{2-[5-dimethylcarbamoyl-3-94-fluoro-phenyl)-4-phenyl-1H-pyrrol-2-yl]-vinyl}-2,2-dimethyl-[1,3]dioxin-4-yl-acetic acid tert-butyl ester, Intermediate 9, dissolved in MeOH (50 mL) was placed in a shaker and 10% palladium on carbon (0.6 g) added. The reaction mixture was placed under hydrogen for 3 h at 50 psi, then filtered. The filtrate was concentrated placed in MeOH and 1.0 N aqueous HCl (7.0 mL) added. The reaction mixture was stirred overnight then concentrated in vacuo. The residue was purified by flash chromatography (0 to 100% EtOAc/Hexane) to give 134 mg (12%) of a white solid. MS(APCI+): m/z 567.3 (M+H); NMR (CDCl₃) δ 1.23-1.50 (7H, m), 1.41(9H, d, J=1.0), 1.61-1.70(5H, m), 2.23-2.34 (3H, m), 2.41(3H, s), 2.48-2.61 (1H, m), 2.83 (3H, s), 3.68-3.79 (1H, m),4.02-4.14 (1H, m),4.51 (1H, sept, J=7.0), 6.84-7.10 (9H, m).

Step F

### Sodium;(3R,5R)-7-[5-dimethylcarbamoylcarboyl-3-(4-fluoro-phenyl)-1-isopropyl-4-phenyl-1H-pyrrol-2-yl]-3,5-dihydroxy-heptanoate

To a solution of (3R,5R)-7-[5-Dimethylcarbamoyl-3-(4-fluoro-phenyl)-1-isopropyl-4-phenyl-1H-pyrrol-2-yl]-3,5-dihydroxy-heptanoic acid tert-butyl ester, Intermediate 10, (33 mg, 0.058 mmol) in a solution of absolute ethanol (5.0 mL) was added 0.1 N aqueous sodium hydroxide solution (0.6 mL) at room temperature. The mixture was stirred for 1 h and then concentrated in vacuo to give a residue, which was dissolved in a solution of 20% methanol in methylene chloride and filtered. The filtrate was concentrated in vacuo to give a solid. The solid was triturated with ether, filtered, and dried in vacuo to give 30 mg (97%) of the desired product as a white solid: MS(APCI⁺): m/z 511.2 (M+1); Anal. Calcd for C₂₉H₃₄FN₂O₅Na.4.25H₂O.0.45CH₂Cl₂: C, 54.64; H, 6.76; N, 4.33. Found: C, 54.26; H, 6.39; N, 3.95.

### Example 177

### Sodium;(3R,5R)-7-[5-carbamoyl-3,4-bis(4-fluoro-phenyl)-1-isopropyl-1H-pyrrol-2-yl]-3,5-dihydroxy-heptanoate

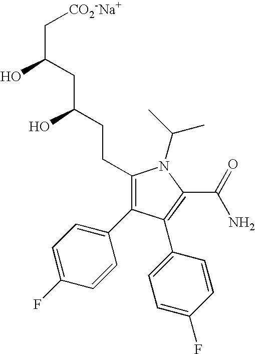

Synthesized in a similar manner to Example 137. MS(APCI⁺): m/z.501.1 (M+1); Anal. Calcd for C₂₇H₂₉F₂N₂O₅Na.1.90H₂O: C, 58.25; H, 5.94; N, 5.03. Found: C, 57.86; H, 5.65; N, 4.87.

### Example 178

### Sodium;(3R,5R)-7-[3,4-bis(4-fluoro-phenyl)-1-isopropyl-5-(6-methoxy-pyridin-2-ylcarbamoyl)-1H-pyrrol-2-yl]-3,5-dihydroxy-heptanoate

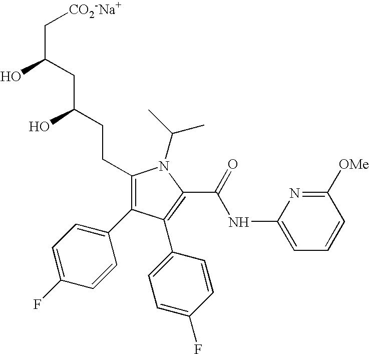 Step A
Intermediate 11

### (4R,6R)-(6-{2-[3,4-bis(4-fluoro-phenyl)-1-isopropyl-5-(6-methoxy-pyridin-2-ylcarbamoyl)-1H-pyrrol-2-yl]-ethyl}-2,2-dimethyl-[1,3]dioxin-yl)-acetic Acid Methyl Ester

Starting from (4R,6R)-(6-{2-[3,4-bis-(4-fluoro-phenyl)-1-isopropyl-1H-pyrrol-2-yl]-ethyl}-2,2-dimethyl-[1,3]dioxan-4-yl)-acetic acid methyl ester (Example 24), this compound was prepared in a similar manner as described for Example 25 (Step D-E).

MS(APCI⁻): m/z 662.3 (M+H); NMR (CDCl3) δ 1.01-1.11 (1H, m), 1.30 (3H, s), 1.34 (3H, s), 1.34-1.60 (3H, m), 1.66 (6H, d, J=7.0), 2.26-2.36 (1H, m), 2.43-2.56 (1H, m),2.60-2.70 (1H, m), 2.80-2.91 (1H, m), 3.56 (3H, s), 3.64 (3H, s), 3.64-3.83 (1H, m), 4.20-4.30 (1H, m), 4.90-5.00 (1H, m), 6.35 (1H, dd, J=8.2, J=0.5) 6.81-6.94 (4H, m), 6.94-6.98 (2H, m), 7.02-7.07 (2H, m), 7.48-7.53 (2H, m), 7.68 (1H, d, J=7.8).

Step B

Intermediate 12

### (3R,5R)-7-[3,4-Bis(4-fluoro-phenyl)-1-isopropyl-5-(6-methoxy-pyridin-2-ylcarbamoyl)-1H-pyrrol-2-yl]-3,5-dihydroxy-heptanoic Acid Methyl Ester

To a solution of (4R,6R)-(6-{2-[3,4-Bis(4-fluoro-phenyl)-1-isopropyl-5-(6-methoxy-pyridin-2-ylcarbamoyl)-1H-pyrrol-2-yl]-ethyl)-2,2-dimethyl-[1,3]dioxin-yl)-acetic acid methyl ester (0.41 g) in MeOH (10 mL) was added 1N aqueous HCl (1.5 mL). The resulting mixture was stirred 4h, then diluted with water and extracted with EtOAc (3×30 mL). The combined extracts were washed with saturated NaHCO3 and brine. The organic phase was allowed to stand overnight (approx 16h) then concentrated and purified by flash chromatography (0 to 100% EtOAc/Hexane) to give 0.186 g of white solid. HPLC purity 96.9%. MS(APCI−): m/z 622.2 (M+H); NMR (CDCl3) δ 1.29-1.60 (6H, m), 1.6z(6H, dd, J=7.0, J=2), 2.37-2.39 (2H, m),2.63-2.67 (1H, m), 2.80-2.91 (1H, m), 3.55 (3Hs), 3.67 (3H, s), 3.67-3.76 (1H, m), 4.10-4.20 (1H, m), 4.90-5.00 (1H, m), 6.35 (1H, dd, J=8.2, J=0.8) 6.82-6.95 (4H, m), 6.96-6.99 (2H, m), 7.02-7.07 (2H, m), 7.48-7.54 (2H, m), 7.67 (1H, d, J=7.4).

Step C

### Sodium;(3R,5R)-7-[3,4-bis(4-fluoro-phenyl)-1-isopropyl-5-(6-methoxy-pyridin-2-ylcarbamoyl)-1H-pyrrol-2-yl]-3,5-dihydroxy-heptanoate

To a solution of the ester (230 mg) in EtOH (10 mL) in an ice-bath was added 0.10N NaOH solution (2.7 mL) dropwise. The resulting mixture is warmed to room temperature and stirred 1 h then concentrated under vacuum. The residue was dissolved in toluene (5 mL) and MeOH (2 mL) then concentrated under vacuum. This procedure was repeated and the residue dissolved in CH₂Cl₂ (5 mL) and MeOH (0.5 mL). This mixture was let stand 1 h then titurated in ether to give 112 mg (84%) of white-powder. MS(APCI⁺): m/z.608.2 (M+1); Anal. Calcd for C₂₇H₂₉F₂N₂O₅Na.3.20H₂O.0.75CH₂Cl₂: C, 53.98; H, 5.62; N, 5.60. Found: C, 53.60; H, 5.26; N, 5.44.

### Example 179

### 3,4-bis-(4-fluoro-phenyl)-5-formyl-1-isopropyl-1H-pyrrole-2-carboxylic Acid (4-methyl-pyrimidin-2-yl)-amide

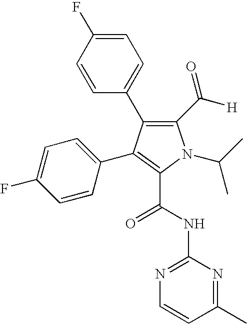 Step A

### 3,4-bis-(4-fluoro-phenyl)-1-isopropyl-1H-pyrrole-2-carboxylic Acid (4-methyl-pyrimidin-2-yl)-amide

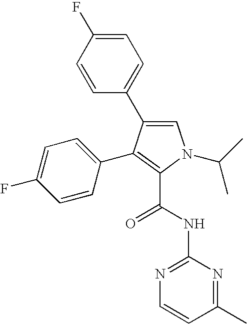

To a mixture of sodium hydride (0.22 g, 60 wt % in mineral oil, 5.4 mmoles) in anhydrous tetrahydrofuran (20 mL) was added 2-amino-4-methylpyrimidine (0.30 g, 2.7 mmoles). The reaction mixture was heated at 60° C. for 30 min and then 3,4-bis-(4-fluoro-phenyl)-1-isopropyl-1H-pyrrole-2-carboxylic acid ethyl ester (prepared in Step C of Example 11) (1.0 g, 2.7 mmoles) was added. The reaction mixture was stirred at reflux for 18 hrs and then poured into a mixture of ice and water (400 mL). The mixture was acidified with 1 N HCl to pH=6 to form a yellow precipitate, which was triturated at room temperature for 3 hrs. The mixture was filtered to collect a crude yellow solid, which was purified by flash chromatography (silica gel, 50-70% ethyl acetate in hexane) to afford 807 mg (69%) of the desired product as awhite solid: mp 165-166° C.; MS (APCI⁺) m/z 433.

Step B

### 3,4-bis-(4-fluoro-phenyl)-5-formyl-1-isopropyl-1H-pyrrole-2-carboxylic Acid (4-methyl-pyrimidin-2-yl)-amide

To a cold (0° C.) solution of 3,4-bis-(4-fluoro-phenyl)-1-isopropyl-1H-pyrrole-2-carboxylic acid (4-methyl-pyrimidin-2-yl)-amide (prepared in Step A of Example PF-00956902) (0.43 g, 1 mmole) and α,α-dichloromethyl methyl ether (0.36 g, 3.1 mmoles) in anhydrous dichloromethane (10 mL) was added 3.6 mL of titanium tetrachloride (0.68 g, 3.6 mmoles) over a 2-min period. The reaction mixture was stirred at room temperature for 6 hrs and then at reflux for 18 hrs. The reaction mixture poured over ice water and then the mixture was extracted with dichloromethane (2×100 mL). The combined organic extracts were washed with saturated sodium bicarbonate (2×100 mL), brine (100 mL), dried (sodium sulfate), filtered, and evaporated to afford a residue, which was purified by flash chromatography (silica gel, 50% ethyl acetate in hexane) to provide 248 mg (54%) of the desired product as an off-white: mp 173-175° C.; MS (APCI⁺) m/z 461.

### Formulations

The compounds of the present invention including those exemplified herein and all compounds of Formula I, hereafter referred to as “compound(s)” can be administered alone or in combination with one or more therapeutic agents. These include, for example, other agents for treating, preventing or controlling dyslipidemia, non-insulin dependent diabetes mellitus, obesity, hyperglycemia, hypercholesteremia, hyperlipidemia, atherosclerosis, hypertriglyceridemia, or hyperinsulinemia.

The compounds are thus well suited to formulation for convenient administration to mammals for the prevention and treatment of such disorders.

The following examples further illustrate typical formulations of the compounds provided by the invention.

Formulation 1

| Ingredient | Amount | |
|---|---|---|
| compound | 0.5 to 800 | mg |
| sodium benzoate | 5 | mg |
| isotonic saline | 1000 | mL |

The above ingredients are mixed and dissolved in the saline for IV administration to a patient.

Formulation 2

| Ingredient | Amount | |
|---|---|---|
| compound | 0.5 to 800 | mg |
| cellulose, microcrystalline | 400 | mg |
| stearic acid | 5 | mg |
| silicon dioxide | 10 | mg |
| sugar, confectionery | 50 | mg |

The ingredients are blended to uniformity and pressed into a tablet that is well suited for oral administration to a patient.

Formulation 3

| Ingredient | Amount | |
|---|---|---|
| compound | 0.5 to 800 | mg |
| starch, dried | 250 | mg |
| magnesium stearate | 10 | mg |

The ingredients are combined and milled to afford material suitable for filling hard gelatin capsules administered to a patient.

Formulation 4

| Ingredient | Amount % wt./(total wt.) |
|---|---|
| compound | 1 to 50 |
| Polyethylene glycol 1000 | 32 to 75 |
| Polyethylene glycol 4000 | 16 to 25 |

The ingredients are combined via melting and then poured into molds containing 2.5 g total weight.

While embodiments of the invention have been illustrated and described, it is not intended that these embodiments illustrate and describe all possible forms of the invention. Rather, the words used in the specification are words of description rather than limitation, and it is understood that various changes may be made without departing from the spirit and scope of the invention.

### Biological Assays

The compounds of the invention have demonstrated HMG Co-A reductase inhibition in standard assays commonly employed by those skilled in the art. (See, e.g., J. of Lipid Research 1998; 39: 75-84; Analytical Biochemistry, 1991; 196: 211-214; RR740-01077 Pharmacology 8, Nov., 1982) Accordingly, such compounds and formulations comprising such compounds are useful for treating, controlling or preventing inter alia hypercholesterolemia, hyperlipidemia, hypertriglyceridemia or atherosclerosis.

A.) In Vitro Assay

Rat Liver Microsomal Isolation Procedure:

Male Charles River Sprague-Dawley rats were fed with 2.5% cholestyramine in rat chow diets for 5 days before sacrificing. Livers were minced and homogenized in a sucrose homogenizing solution in an ice bath 10 times. Homogenates were diluted into a final volume of 200 mL, and centrifuged 15 min. with a Sorvall Centrifuge at 5° C., 10,000 rpm (12,000×G). The upper fat layer was removed and the supernatant decanted into fresh tubes. This step was repeated one more time before transferring the supernatant into ultracentrifuge tubes and centrifuged at 36,000 rpm (105,000×G) for an hour at 5° C. The resulting supernatant was discarded and the pellet was added to total of 15 mL 0.2 M KH₂PO₄. Pellets were homogenized gently by hand about 10 times. Samples were pooled and diluted into total of 60 mL buffer. The protein concentration of the homogenate was determined by the Lowry Method using a BCA kit from Pierce Chemical Company. 1 mL aliquots of microsomes were kept frozen in liquid nitrogen.

HMGCoA (3-Hydroxy-3-methylglutaryl CoA) Reductase Assay:

Materials and Methods:

[3-¹⁴C]-HMGCoA (57.0 mCi/mmol) was purchased from Amersham Biosciences, UK. HMGCoA, mevalonolactone, NADPH were purchased from Sigma Chemical Co. AG 1-8X resin was purchased from Bio-Rad Laboratory.

One μL of dimethyl sulfoxide (DMSO) or 1 μL of DMSO containing a test compound at a concentration sufficient to give a final assay concentration of between 0.1 nM to 1 mM was placed into each well of a Corning 96 well plate. A Volume of 34 μL of buffer (100 mM NaH₂PO₄, 10 mM Imidazole and 10 mM EDTA) containing with 50 μg/mL rat liver microsomes was added into each well. After incubation for 30 min. on ice, 15 μL of ¹⁴C-HMGCoA (0.024 μCi) with 15 mM NADPH, 25 mM DTT was added and incubated at 37° C. for an additional 45 min. The reaction was terminated by the addition of 10 μL of HCl followed by 5 μL of mevalonolactone. Plates were incubated at room temperature overnight to allow lactonization of mevalonate to mevalonolactone. The incubated samples were applied to columns containing 300 μL of AG1-X8 anion exchange resin in a Corning filter plate. The eluates were collected into Corning 96 well capture plates. Scintillation cocktail (Ultima-Flo-M) was added into each well and plates counted on a Trilux Microbeta Counter. The IC₅₀ values were calculated with GraphPad software (Prism).

Procedure:

1. Add 1 μL DMSO or compounds into the wells according to the protocol
2. Add 35 μL incubation buffer with the rat microsomes into each well. Incubate 30 min. at 4° C.
3. Add 15 μL ¹⁴C-HMGCoA. Incubate 45 min. at 37° C.
4. Add 10 μL HCl stop reagent
5. Add 5 μL mevelonolactone. Incubate overnight at room temperature
6. Apply the containing into the AG 1-X8 anion exchange resin in Corning filter plate
7. Collect the eluate into Corning capture plate
8. Add scintillation cocktail Ultima-Flo-M
9. Count on a Trilux Microbeta Counter
10. Calculate IC₅₀ values

Compounds of the invention exhibit a range of IC₅₀ values of less than about 500 nM. Preferred compounds of the invention exhibit a range of IC₅₀ values of less than about 100 nM. More preferred compounds of the invention exhibit a range of IC₅₀ values of less than about 20 nM. See, for example, Example 1 which has an IC₅₀ of 12 nM, Example 6 which has an IC₅₀ of 4.1 nM, and Example 25 which has an IC₅₀ of 0.61 nM.

B.) Cell Assay

Protocol for Sterol Biosynthesis in Rat Hepatocytes:

Cell Culture, Compounds Treatment and Cell Labeling:

Frozen rat hepatocytes purchased from XenoTech(cat# N400572) were seeded on 6-well collagen I coated plates at a density of 10 cells/per well. The cells were grown in DMEM medium (Gibco, #11054-020) containing 10% FBS and 10 mM HEPES(Gibco # 15630-080) for 24 hrs. The cells were pre-incubated with compounds for 4 hrs and then labeled by incubating in medium containing 1 uCi/per ml of ¹⁴C acetic acid for an additional 4 hrs. After labeling, the cells were washed twice with 5 mM MOPS solution containing 150 mM NaCl and 1 mM EDTA and collected in the lysis buffer containing 10% KOH and 80%(vol.) ethanol.

Cholesterol Extraction and Data Analysis:

In order to separate labeled cholesterol from labeled non-cholesterol lipids, the cells lysates were subject to saponification at 60° C. for 2 hrs. The lysates were then combined with 0.5 volume of H₂O and 2 volumes of hexane, followed by 30 minutes of vigorous shaking. After the separation of two phases, the upper-phase solution was collected and combined with 5 volumes of scintillation cocktail. The amount of ¹⁴C cholesterol was quantified by liquid scintillation counting. The IC₅₀ values were calculated with GraphPad software (Prism 3.03). Compounds of the invention exhibit a range of IC₅₀ values of less than about 1000 nM. Preferred compounds of the invention exhibit a range of IC₅₀ values of less than about 100 nM. See, for example, Example 1 which has an IC₅₀ of 0.74 nM, Example 6 which has an IC₅₀ of 0.23 nM, and Example 25 which has an IC₅₀ of 0.19 nM.

C.) Protocol for Sterol Biosynthesis in L6 Rat Myoblast:

Cell Culture, Compounds Treatment and Cell Labeling:

L6 rat myoblast purchased from ATCC(CRL-1458) were grown in T-150 vented culture flasks and seeded on 12-well culture plates at a density of 60,000 cells per well. The cells were grown in DMEM, (Dulbecco's Modified Eagle Medium) (Gibco, #10567-014) containing 10% heat inactivated FBS (Fetal Bovine Serum) (Gibco # 10082-139) for 72 hours until reaching confluence. The cells were pre-incubated in media with compound and 0.2% DMSO (dimethyl sulfoxide) for 3 hours and then labeled by incubating in medium containing compound, 0.2% DMSO and 1 μCi/per mL of ¹⁴C acetic acid for an additional 3 hours. After labeling, the cells were washed once with 1×PBS (Gibco #14190-144) then lysed overnight at 4° C. in buffer containing 10% KOH and 78%(vol.) ethanol.

Cholesterol Extraction and Data Analysis:

Lipid ester bonds were hydrolyzed by saponification of the lysates at 60° C. for 2 hours. Sterols (including cholesterol) were extracted from saponified lysates by combining with 3 volumes of hexane and mixing by pipette 6 times. The upper organic phase solution was collected and combined with an equal volume of 1N KOH in 50% methanol and mixed by pipette 6 times. The upper organic phase was collected in a scintilant-coated plate (Wallac #1450-501) and hexanes removed by evaporation at room temperature for 3 hours. The amount of ¹⁴C cholesterol was quantified by scintillation counting in a Trilux 1450 plate reader (Wallac). The IC₅₀ values were calculated from % inhibitions relative to negative controls vs. compound concentration on Microsoft excel 2000 data analysis wizard using a sigmoid inhibition curve model with formula:
y=Bmax(1−(xⁿ/Kⁿ+xⁿ))+y2
Where K is the IC₅₀ for the inhibition curve, X is inhibitor concentration, Y is the response being inhibited and Bmax+Y2 is the limiting response as X approaches zero. Compounds of the invention have a L6 IC₅₀ value greater than 0.5 nM. See, for example, the compound of Example 1, which as an L6 IC₅₀ of 157 nM, and the compound of Example 25, which has an L6 IC₅₀ of 2270 nM.

Preferred compounds of the invention exhibit a hepatocyte selectivity greater than 1000 ((L6 IC₅₀/Rat hepatocyte IC₅₀)>1000), and have a L6 IC₅₀ value greater than 1 nM.

1. A compound having a Formula I, 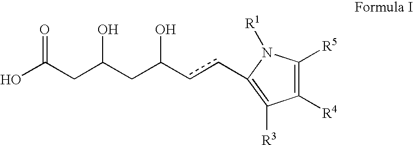
or a pharmaceutically acceptable salt, ester, amide, stereoisomer or prodrug thereof, or a pharmaceutically acceptable salt of the prodrug,
wherein R¹ is lower alkyl, optionally substituted with a halogen;
R³ is benzyl; naphthyl; C₃-C₈ cycloalkyl or C₅-C₈ cycloalkenyl, optionally one or more heteroatom(s); phenyl or phenyl substituted with one or more groups selected from fluorine, chlorine, bromine, hydroxyl or alkyl of from one to seven carbon atoms; pyridinyl or pyridinyl substituted with fluorine, chlorine, bromine, hydroxyl or alkyl of from one to seven carbon atoms;
R⁴ is H; aryl, aralkyl, heteroaryl or heteroaralkyl; optionally substituted with one or more groups selected from fluorine, chlorine, bromine, hydroxyl, (CH₂)ₙOR′, (CH₂)ₙCOOR′, (CH₂)ₙCONR′R″,
(CH₂)ₙS(O)₂NR′R″, (CH₂)ₙS(O)₂R⁸, alkyl or alkoxy of from one to seven carbon atoms; C₁-C₈ alkyl or C₃-C₈ cycloalkyl; optionally substituted; aralkenyl; carbamoyl or substituted carbamoyl; carboxyl or substituted carboxyl;
R⁵ is H, I, phenyl or substituted phenyl, COOR′, R⁶R⁷NC(O)—;
—(CH₂)ₙN R⁶R⁷ or SO₂NR⁶R⁷;
R⁶ and R⁷ are each independently H; aryl, aralkyl, heteroaryl or heteroaralkyl; optionally substituted with halo, alkyl of from one to seven carbon atoms, (CH₂)ₙOR′, (CH₂)ₙCOOR′, (CH₂)ₙCONR′R″,
(CH₂)ₙS(O)₂NR′R″, (CH₂)ₙS(O)₂R⁸, or heteroaryl;
C₁-C₁₀ alkyl, C₃-C₈ cycloalkyl or C₅-C₈ cycloalkenyl, said alkyl, cycloalkyl or cycloalkenyl optionally containing one or more heteroatoms(s); unsubstituted or substituted with OH, CO₂R′ or
CONR′R″;
COOR′; C(O)R′; SO₂NHR⁸ or SO₂R⁸;
or N, R⁶ and R⁷ taken together form a 4-7 member ring, optionally containing up to 2 heteroatoms selected from O, N and S, said heteroatom(s) being optionally substituted; said ring optionally substituted with lower alkyl, OH, benzyl, phenyl, CO₂R′ or CONR′R″;
R ⁸ is aryl, aralkyl, alkyl, heteroaryl or heteroaralkyl; optionally substituted;
R′ and R″ are each independently H, C₁-C₁₂ alkyl, aryl, or aralkyl, or taken together form a 4-7 member ring;
n is 0-2; and
wherein 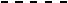
is a bond or is absent.

2. A stereoisomer of a compound of claim 1 comprising a (3R,5R)-isomer.

3. A stereoisomer of a compound of claim 1 comprising a (3R,5S)-isomer.

4. A stereoisomer of a compound of claim 1 comprising a (3S,5S)-isomer.

5. A stereoisomer of a compound of claim 1 comprising a (3S,5R)-isomer.

6. A compound of claim 1 or the pharmaceutically acceptable salt, ester, amide, stereoisomer or prodrug thereof, or the pharmaceutically acceptable salt of the prodrug, wherein R³ is phenyl or substituted phenyl, or pyridinyl or substituted pyridinyl.

7. A compound of claim 6 or the pharmaceutically acceptable salt, ester, amide, stereoisomer or prodrug thereof, or the pharmaceutically acceptable salt of the prodrug, wherein R³ is phenyl substituted with fluorine, chlorine or bromine.

8. A compound of claim 7 or the pharmaceutically acceptable salt, ester, amide, stereoisomer or prodrug thereof, or the pharmaceutically acceptable salt of the prodrug, wherein R³ is para-fluorophenyl.

9. A compound of claim 1 or the pharmaceutically acceptable salt, ester, amide, stereoisomer or prodrug thereof, or the pharmaceutically acceptable salt of the prodrug, wherein R⁴ is phenyl, biphenyl or substituted phenyl; pyridinyl or substituted pyridinyl; C₁-C₈ alkyl optionally substituted; or naphthyl.

10. A compound of claim 1 or the pharmaceutically acceptable salt, ester, amide, stereoisomer or prodrug thereof, or the pharmaceutically acceptable salt of the prodrug, wherein R⁴ is cyclohexyl-, clyclopentyl-, cyclobutyl-, cyclopropyl-, methyl-, ethyl-, isopropyl-, difluoromethyl, trifluoro-methyl or phenyl substituted with one or more halogen.

11. A compound of claim 9 or the pharmaceutically acceptable salt, ester, amide, stereoisomer or prodrug thereof, or the pharmaceutically acceptable salt of the prodrug, wherein R⁴ is phenyl or para-fluorophenyl.

12. A compound of claim 1 or the pharmaceutically acceptable salt, ester, amide, stereoisomer or prodrug thereof, or the pharmaceutically acceptable salt of the prodrug, wherein R¹ is C₁-C₄ alkyl.

13. A compound of claim 12 or the pharmaceutically acceptable salt, ester, amide, stereoisomer or prodrug thereof, or the pharmaceutically acceptable salt of the prodrug, wherein R¹ is ethyl or propyl.

14. A compound of claim 12 or the pharmaceutically acceptable salt, ester, amide, stereoisomer or prodrug thereof, or the pharmaceutically acceptable salt of the prodrug, wherein R⁵ is SO₂NR⁶R⁷; —(CH₂)ₙNR⁶R⁷; or
R⁶R⁷NC(O)—; R⁴ is phenyl, para-fluorophenyl, isopropyl, cyclopropyl, methyl, ethyl, CHF₂ or CF₃; and R³ is phenyl or para-fluorophenyl.

15. A compound of claim 14 or the pharmaceutically acceptable salt, ester, amide, stereoisomer or prodrug thereof, or the pharmaceutically acceptable salt of the prodrug, wherein R⁶ and R⁷ are each independently H; methyl;
phenyl or phenyl substituted with halo, alkyl of from one to seven carbon atoms, (CH₂)ₙOR′, (CH₂)ₙCOOR′, (CH₂)ₙCONR′R″, (CH₂)ₙS(O)₂NR′R″, (CH₂)ₙS(O)₂R⁸ or heteroaryl; or
benzyl or benzyl substituted with halo, alkyl of from one to seven carbon atoms, (CH₂)ₙOR′, (CH₂)ₙCOOR′, (CH₂),CONR′R″, (CH₂)ₙS(O)₂NR′R″, (CH₂)ₙS(O)₂R⁸, or heteroaryl.

16. A compound of claim 1 or the pharmaceutically acceptable salt, ester, amide, stereoisomer or prodrug thereof, or the pharmaceutically acceptable salt of the prodrug, wherein R¹ is isopropyl, ethyl, trifluoromethyl, difluoromethyl or cyclopropyl.

17. A compound of claim 16 or the pharmaceutically acceptable salt, ester, amide, stereoisomer or prodrug thereof, or the pharmaceutically acceptable salt of the prodrug, wherein R¹ is isopropyl and R³ is para-fluorophenyl.

18. A pharmaceutically acceptable salt of a compound of claim 1 wherein the salt is a sodium salt or a calcium salt.

19. A pharmaceutically acceptable ester of claim 1 wherein the ester is a methyl ester or ethyl ester.

20. A compound of claim 1, the pharmaceutically acceptable salt, ester, amide, stereoisomer or prodrug thereof, or the pharmaceutically acceptable salt of the prodrug wherein R⁶ and R⁷ are each independently H, phenyl or substituted phenyl, benzyl or substituted benzyl phenyl-ethyl, pyridinyl or substituted pyridinyl or C₁-C₄ alkyl.

21. A compound of claim 13 wherein R¹ is isopropyl.

22. A compound of claim 1 or the pharmaceutically acceptable salt, ester, amide, stereoisomer or prodrug thereof, or the pharmaceutically acceptable salt of the prodrug, wherein R⁴ and R³ are each independently phenyl or substituted phenyl and R¹ is C₁-C₄ alkyl.

23. A compound of claim 22 or the pharmaceutically acceptable salt, ester, amide, stereoisomer or prodrug thereof, or the pharmaceutically acceptable salt of the prodrug, wherein R⁵ is SO₂NR⁶R⁷; —(CH₂)ₙNR⁶R⁷ or R⁶R⁷NC(O)—.

24. A compound of claim 1 or the pharmaceutically acceptable salt, ester, amide, stereoisomer or prodrug thereof, or the pharmaceutically acceptable salt of the prodrug, wherein R⁶ and R⁷ are each independently H, Me, phenyl or phenyl substituted with halo alkyl of from one to seven carbon atoms, (CH₂)ₙOR′, (CH₂)ₙCOOR′, (CH₂)ₙCONR′R″, (CH₂)ₙS(O)₂NR′R″, (CH₂)ₙS(O)₂R⁸, or heteroaryl; or
benzyl or benzyl substituted with halo, alkyl of from one to seven carbon atoms, (CH₂)ₙOR′, (CH₂)ₙCOOR′, (CH₂)ₙCONR′R″, (CH₂)ₙS(O)₂NR′R″, (CH₂)ₙS(O)₂R⁸, or heteroaryl.

25. A compound of claim 1 or the pharmaceutically acceptable salt, ester, amide, stereoisomer or prodrug thereof, or the pharmaceutically acceptable salt of the prodrug, wherein at least one of R⁶ or R⁷ is SO₂NHR⁸ or SO₂R⁸ and R⁸ is phenyl or substituted phenyl.

26. A compound of claim 1 wherein N, R⁶ and R⁷ taken together form a 4-7 member ring, optionally containing up to 2 heteroatoms selected from O, N, and S, said up to 2 heteroatoms being optionally substituted; said ring optionally substituted with lower alkyl, OH, benzyl, phenyl, CO₂R′ or CONR′R″; and R′ and R″ are each independently H, C₁₋₁₂ allkyl, aryl, or taken together form a 4-7 member ring.

27. A compound of claim 26 wherein N, R⁶ and R⁷ taken together form a 4-7 member ring, said ring optionally substituted with lower alkyl, phenyl or benzyl.

28. A compound of claim 1 or the pharmaceutically acceptable salt, ester, amide, stereoisomer or prodrug thereof, or the pharmaceutically acceptable salt of the prodrug, wherein R⁴ is carbamoyl substituted with phenyl, said phenyl being optionally substituted with CONR′R″.

29. A compound of claim 1 or claim 14 wherein R⁵ is SO₂NR⁶R⁷.

30. A compound of claim 1 or claim 14 wherein R⁵ is R⁶R⁷NC(O)— or —(CH₂)ₙ NR⁶R⁷.

31. A compound of claim 1 wherein R¹ is C₂-C₃ alkyl; R³ and R⁴ are each independently phenyl or para-fluorophenyl; and R⁵ is H, I, phenyl, COOR′, R⁶R⁷NC(O); (CH₂)ₙNR⁶R⁷— or SO₂NR⁶R⁷.

32. A pharmaceutical composition comprising a compound of claim 1 or the pharmaceutically acceptable salt, ester, amide or prodrug thereof, or the pharmaceutically acceptable salt of the prodrug; or a mixture thereof; and a pharmaceutically acceptable carrier, diluent or vehicle.

33. A method of inhibiting cholesterol biosynthesis in a mammal requiring inhibition comprising administering to the mammal a therapeutically effective amount of a compound of claim 1 or the pharmaceutically acceptable salt, ester, amide or prodrug thereof, or the pharmaceutically acceptable salt of the prodrug.

34. A method of lowering LDL cholesterol in a mammal comprising administering to the mammal in need thereof a therapeutically effective amount of a compound of claim 1 or the pharmaceutically acceptable salt, ester, amide or prodrug thereof, or the pharmaceutically acceptable salt of the prodrug.

35. A method of raising HDL cholesterol in a mammal comprising administering to the mammal in need thereof a therapeutically effective amount of a compound of claim 1 or the pharmaceutically acceptable salt, ester, amide or prodrug thereof, or the pharmaceutically acceptable salt of the prodrug.

36. A method of treating, preventing or controlling hyperlipidemia in a mammal comprising administering to the mammal in need thereof a therapeutically effective amount of a compound of claim 1 or the pharmaceutically acceptable salt, ester, amide or prodrug thereof, or the pharmaceutically acceptable salt of the prodrug.

37. A method of treating, preventing or controlling hypercholesterolemia in a mammal comprising administering to the mammal in need thereof a therapeutically effective amount of a compound of claim 1 or the pharmaceutically acceptable salt, ester, amide or prodrug thereof, or the pharmaceutically acceptable salt of the prodrug.

38. A method of treating, preventing or controlling hypertriglyceridemia in a mammal comprising administering to the mammal in need thereof a therapeutically effective amount of a compound of claim 1 or the pharmaceutically acceptable salt, ester, amide or prodrug thereof, or the pharmaceutically acceptable salt of the prodrug.

39. A method of treating, preventing or controlling atherosclerosis in a mammal comprising administering to the mammal in need thereof a therapeutically effective amount of a compound of claim 1 or the pharmaceutically acceptable salt, ester, amide or prodrug thereof, or the pharmaceutically acceptable salt of the prodrug.

40. A method of treating, preventing or controlling Alzheimer's disease, BPH, diabetes or osteoporosis in a mammal comprising administering to the mammal in need thereof a therapeutically effective amount of a compound of claim 1 or the pharmaceutically acceptable salt, ester, amide or prodrug thereof, or the pharmaceutically acceptable salt of the prodrug.

41. A compound selected from the group consisting of: (3R,5R)-7-[3-(4-fluoro-phenyl)-1-isopropyl-4-phenyl-5-phenylcarbamoyl-1H-pyrrol-2-yl]-3,5-dihydroxy-heptanoic acid;
(3R,5 S)-7-[3-(4-Fluoro-phenyl)-1-isopropyl-4-phenyl-5-phenylcarbamoyl-1H-pyrrol-2-yl]-3,5-dihydroxy-hept-6-enoic acid;
(3R)-7-[3-(4-Fluoro-phenyl)-1-isopropyl-4-phenyl-5-phenylcarbamoyl-1H-pyrrol-2-yl]-3,5-dihydroxy-heptanoic acid;
(3R,5R)-7-[3-(4-Fluoro-phenyl)-1-isopropyl-4-phenyl-5-(4-sulfamoyl-phenylcarbamoyl)-1H-pyrrol-2-yl]-3,5-dihydroxy-heptanoic acid;
(3R,5 S)-7-[3-(4-Fluoro-phenyl)-1-isopropyl-4-phenyl-5-(4-sulfamoyl-phenylcarbamoyl)-1H-pyrrol-2-yl]-3,5-dihydroxy-hept-6-enoic acid;
(3R,5R)-7-[3-(4-Fluoro-phenyl)-5-(4-fluoro-phenylcarbamoyl)-1-isopropyl-4-phenyl-1H-pyrrol-2-yl]-3,5-dihydroxy-heptanoic acid;
(3R,5 S)-7-[3-(4-Fluoro-phenyl)-5-(4-fluoro-phenylcarbamoyl)-1-isopropyl-4-phenyl-5-phenylcarbamoyl-1H-pyrrol-2-yl]-3,5-dihydroxy-hept-6-enoic acid; (3R)-7-[3-(4-Fluoro-phenyl)-5-(4-fluoro-phenylcarbamoyl)-1-isopropyl-4-phenyl-1H-pyrrol-2-yl]-3,5-dihydroxy-heptanoic acid;
(3R,5R)-7-[5-(4-Fluoro-benzylcarbamoyl)-3-(4-fluoro-phenyl)-1-isopropyl-4-phenyl-1H-pyrrol-2-yl]-3,5-dihydroxy-heptanoic acid;
(3R,5S)-7-[5-(4-Fluoro-benzylcarbamoyl)-3-(4-fluoro-phenyl)-1-isopropyl-4-phenyl-1H-pyrrol-2-yl]-3,5-dihydroxy-hept-6-enoic acid;
(3R,5R)-7-[3,4-Bis-(4-fluoro-phenyl)-1-isopropyl-5-(4-sulfamoyl-phenylcarbamoyl)-1H-pyrrol-2-yl]-3,5-dihydroxy-heptanoic acid;
(3R,5S)-7-[3,4-Bis-(4-fluoro-phenyl)-1-isopropyl-5-(4-sulfamoyl-phenycarbamoyl-1H-pyrrol-2-yl]-3,5-dihydroxy-hept-6-enoic acid;
(3R,5R)-7-[5-(4-Carbamoylmethyl-phenylcarbamoyl)-3,4-bis-(4-fluoro-phenyl)-1-isopropyl-)-1H-pyrrol-2-yl]-3,5-dihydroxy-heptanoic acid;
(3R,5S)-7-[5-(4-Carbamoylmethyl-phenylcarbamoyl)-3,4-bis-(4-fluoro-phenyl)-1-isopropyl-1H-pyrrol-2-yl]-3,5-dihydroxy-hept-6-enoic acid;
(3R,5R)-7-[3,4-bis(4-fluorophenyl)-1-isopropyl-5-phenylcarbamoyl-1H-pyrrol-2-yl]-3,5-dihydroxy-heptanoic acid; and pharmaceutically acceptable salts, esters and amides thereof.

42. A racemic mixture comprising a compound of claim 1.

43. A compound having a formula, 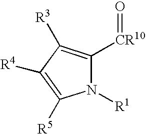
or a pharmaceutically acceptable salt, ester, amide, stereoisomer or prodrug thereof, or a pharmaceutically acceptable salt of the prodrug,
wherein R¹ is lower alkyl, optionally substituted with a halogen;
R³ is benzyl; naphthyl; C₃-C₈ cycloalkyl or C₅-C₈ cycloalkenyl, optionally substituted with one or more heteroatom(s); phenyl or phenyl substituted with one or more groups selected from fluorine, chlorine, bromine, hydroxyl or alkyl of from one to seven carbon atoms; pyridinyl or pyridinyl substituted with fluorine, chlorine, bromine, hydroxyl or alkyl of from one to seven carbon atoms;
R⁴ is H; aryl, aralkyl, heteroaryl or heteroaralkyl; optionally substituted with one or more groups selected from fluorine, chlorine, bromine, hydroxyl or alkyl of from one to seven carbon atoms;
C₁-C₈ alkyl or C₃-C₈ cycloalkyl; optionally substituted; aralkenyl; carbamoyl or substituted carbamoyl; carboxyl or substituted carboxyl;
R⁵ is H, I, phenyl, COOR′, R⁶R⁷NC(O)— or SO₂NR⁶R⁷;
R⁶ and R⁷ are each independently H; aryl, aralkyl, heteroaryl or heteroaralkyl; optionally substituted with halo, alkyl of from one to seven carbon atoms, (CH₂)ₙOR′, (CH₂)ₙCOOR′, (CH₂)ₙCONR′R″,
(CH₂)ₙS(O)₂NR′R″, (CH₂),S(O)₂R⁸, or heteroaryl;
C₁-C₁₀ alkyl, C₃-C₈ cycloalkyl or C₅-C₈ cycloalkenyl, said alkyl, cycloalkyl or cycloalkenyl optionally containing one or more heteroatoms(s); unsubstituted or substituted with OH, CO₂R′ or CONR′R″;
COOR′; C(O)R′; SO₂NHR⁸ or SO₂R⁸;
or N, R⁶ and R⁷ taken together form a 4-7 member ring, optionally containing up to 2 heteroatoms selected from O, N and S, said heteroatom(s) being optionally substituted; said ring optionally substituted with lower alkyl, OH, benzyl, phenyl, CO₂R′ or CONR′R″;
R⁸ is aryl, aralkyl, alkyl, heteroaryl or heteroaralkyl; optionally substituted;
R¹⁰ is H, OH, OC₁-C₆ alkyl; R′ and R″ are each independently H, C₁-C₁₂ alkyl, aryl, or alkyl or taken together form a 4-7 member ring;
n is 0-2; and
wherein 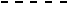
is a bond or is absent.

44. A compound of claim 19 having a Formula 19, 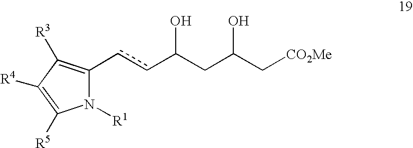
or a pharmaceutically acceptable salt, ester, amide, stereoisomer, racemic mixture or prodrug thereof, or a pharmaceutically acceptable salt of the prodrug, wherein R¹, R³, R⁴ and R⁵ are as defined in claim 19 and Me is methyl.

45. A compound having a formula 21, 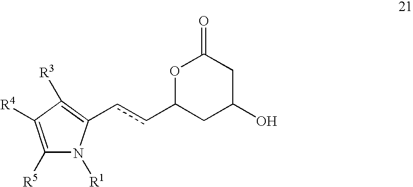
or a pharmaceutically acceptable salt, ester, amide, stereoisomer, racemic mixture or prodrug thereof, or a pharmaceutically acceptable salt of the prodrug, wherein R¹ is lower alkyl, optionally substituted with a halogen;
R³ is benzyl; naphthyl; C₃-C₈ cycloalkyl or C₅-C₈ cycloalkenyl, optionally substituted with one or more heteroatom(s); phenyl or phenyl substituted with one or more groups selected from fluorine, chlorine, bromine, hydroxyl or alkyl of from one to seven carbon atoms; pyridinyl or pyridinyl substituted with fluorine, chlorine, bromine, hydroxyl or alkyl of from one to seven carbon atoms;
R⁴ is H; aryl, aralkyl, heteroaryl or heteroaralkyl; optionally substituted with one or more groups selected from fluorine, chlorine, bromine, hydroxyl or alkyl of from one to seven carbon atoms;
C₁-C₈ alkyl or C₃-C₈ cycloalkyl; optionally substituted; aralkenyl; carbamoyl or substituted carbamoyl; carboxyl or substituted carboxyl;
R⁵ is H, I, phenyl, COOR′, R⁶R⁷NC(O)— or SO₂NR⁶R⁷;
R⁶ and R⁷ are each independently H; aryl, aralkyl, heteroaryl or heteroaralkyl; optionally substituted with halo, alkyl of from one to seven carbon atoms, (CH₂)ₙOR′, (CH₂)ₙCOOR′, (CH₂)ₙCONR′R″, (CH₂)ₙS(O)₂NR′R″, (CH₂)ₙS(O)₂R⁸, or heteroaryl;
C₁-C₁₀ alkyl, C₃-C₈ cycloalkyl or C₅-C₈ cycloalkenyl, said alkyl, cycloalkyl or cycloalkenyl optionally containing one or more heteroatoms(s); unsubstituted or substituted with OH, CO₂R′ or CONR′R″;
COOR′; C(O)R′; SO₂NHR⁸ or SO₂R⁸;
or N, R⁶ and R⁷ taken together form a 4-7 member ring, optionally containing up to 2 heteroatoms selected from O, N and S, said heteroatom(s) being optionally substituted; said ring optionally substituted with lower alkyl OH, benzyl, phenyl, CO₂R′ or CONR′R″;
R⁸ is aryl, aralkyl, alkyl, heteroaryl or heteroaralkyl; optionally substituted;
R′ and R″ are each independently H, C₁-C₁₂ alkyl, aryl, or alkyl or taken together form a 4-7 member ring;
n is 0-2; and
wherein 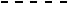
is a bond or is absent.

46. A process for making a compound of claim 43 having a formula, 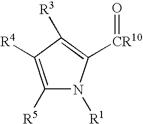
wherein R¹, R³, R⁴, R⁵ and R⁹ are as defined in claim 43 comprising the following steps:
1). Reacting a compound having a formula 5, 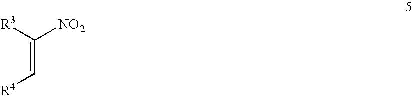
wherein R³ and R⁴ are as defined in claim 39, in a solvent, with ethyl isocyanoacetate to form a compound 6, 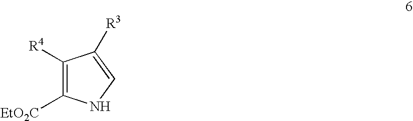
wherein R³ and R⁴ are as defined above and Et is ethyl;
2.) Alkylating the compound 6 to form a compound 7, 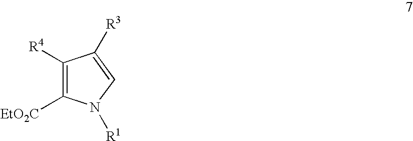
wherein R¹, R³, R⁴ and Et are as defined above; and
3.) Formylating the compound 7 to form the compound.

47. A process for making a stereoisomer of a compound of claim 44 having a formula 19, 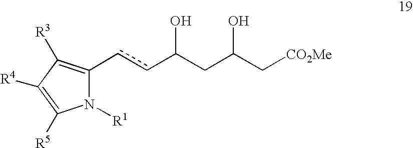
wherein R¹, R³, R⁴ and R⁵ are as defined in claim 42 and Me is methyl, comprising the following steps:
1.) Reacting a compound 10, wherein R¹, R³, R⁴ and R⁵ are as defined above, 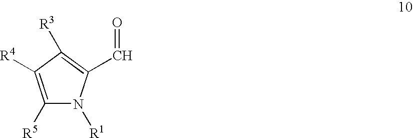
with a compound 11, 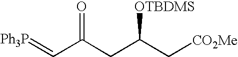
wherein Me is methyl, TBDMS is tert-butyldimethyl-silyl, Ph is phenyl and P is phosphorus, to form a compound 12, 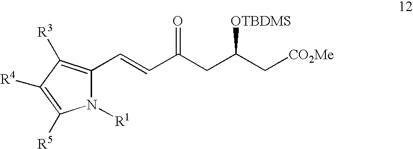
wherein R, R³, R⁴, R⁵, TBDMS and Me are as defined above, 2.) Optionally hydrogenating the compound 12; 3.) Deprotecting the compound 12 to form a steroisomer of a compound 18, 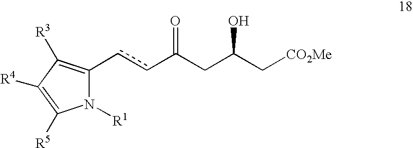
wherein R¹ R³, R⁴, R⁵ and Me are as defined above; and
4.) Stereoselectively reducing the stereoisomer of compound 18 to form the stereoisomer of the compound 19.

48. A compound selected from the group consisting of: (3R,5R)-7-[3,4-bis(4-fluorophenyl)-5-(2-fluoro-phenylcarbamoyl)-1-isopropyl-1H-pyrrol-2-yl]-3,5-dihydroxy-heptanoic acid;
(3R,5S)-7-[3,4-bis(4-fluorophenyl)-5-(4-fluorophenylcarbamoyl)-1-isopropyl-1H-pyrrol-2-yl]-3,5-dihydroxy-hept-6-enoic acid;
(3R,5R)-7-[5-(2,4-difluoro-phenylcarbamoyl)-3,4-bis(4-fluoro-phenyl)-1-isopropyl-1H-pyrrol-2-yl]-3,5-dihydroxy-heptanoic acid;
(3R,5R)-7-[3,4-bis-(4-fluorophenyl)-1-isopropyl-5-p-tolylcarbamoyl-1H-pyrrol-2-yl]-3,5-dihydroxy-heptanoic acid;
(3R,5R)-7-[3,4-bis(4-fluorophenyl)-1-isopropyl-5-m-tolylcarbamoyl-1H-pyrrol-2-yl]-3,5-dihydroxy-heptanoic acid;
(3R,5R)-7-[1-Ethyl-3-(4-fluoro-phenyl)-4-isopropyl-5-phenylcarbamoyl-1H-pyrrol-2-yl]-3,5-dihydroxy-heptanoic acid;
(3R,5R)-7-[1-ethyl-3-(4-fluoro-phenyl)-4-methyl-5-phenylcarbamoyl-1-Hpyrrol-2-yl]-3,5-dihydroxy-heptanoic acid;
(3R,5R)-7-[3,4-bis(4-fluorophenyl)-1-isopropyl-5-(piperidine-1-carbonyl)-1H-pyrrol-2-yl]-3,5-dihydroxy-heptanoic acid; and pharmaceutically acceptable salts, esters and amides thereof.

49. A compound selected from the group consisting of: (3R,5R)-7-[3,4-Bis-(4-fluoro-phenyl)-1-isopropyl-5-(4-methanesulfonyl-benzylcarbamoyl)-1H-pyrrol-2-yl]-3,5-dihydroxy-heptanoic acid;
(3R,5R)-7-[3,4-Bis-(4-fluoro-phenyl)-1-isopropyl-5-(4-sulfamoyl-benzylcarbamoyl)-1H-pyrrol-2-yl]-3,5-dihydroxy-heptanoic acid;
trans-(3R,5S)-7-[3,4-Bis-(4-fluoro-phenyl)-1-isopropyl-5-(4-sulfamoyl-benzylcarbamoyl)-1H-pyrrol-2-yl]-3,5-dihydroxy-hept-6-enoic acid;
(3R,5R)-7-[5-(4-Dimethylcarbamoyl-benzylcarbamoyl)-3,4-bis-(4-fluoro-phenyl)-1-isopropyl-1H-pyrrol-2-yl]-3,5-dihydroxy-heptanoic acid;
(3R,5R)-7-[3,4-Bis-(4-fluoro-phenyl)-1-isopropyl-5-(4-sulfamoylmethyl-phenylcarbamoyl)-1H-pyrrol-2-yl]-3,5-dihydroxy-heptanoic acid;
trans-(3R,5S)-7-[3,4-Bis-(4-fluoro-phenyl)-1-isopropyl-5-(4-sulfamoylmethyl-phenylcarbamoyl)-1H-pyrrol-2-yl]-3,5-dihydroxy-hept-6-enoic acid;
(3R,5R)-7-[3,4-Bis-(4-fluoro-phenyl)-1-isopropyl-5-(4-methanesulfonylmethyl-phenylcarbamoyl)-1H-pyrrol-2-yl]-3,5-dihydroxy-heptanoic acid;
trans-(3R,5S)-7-[3,4-Bis-(4-fluoro-phenyl)-1-isopropyl-5-(4-methanesulfonylmethyl-phenylcarbamoyl)-1H-pyrrol-2-yl]-3,5-dihydroxy-hept-6-enoic acid;
(3R,5R)-7-{3-(4-Fluoro-phenyl)-1-isopropyl-4-phenyl-5-[(pyridin-2-ylmethyl)-carbamoyl]-1H-pyrrol-2-yl}-3,5-dihydroxy-heptanoic acid;
trans-(3R,5S)-7-{3-(4-Fluoro-phenyl)-1-isopropyl-4-phenyl-5-[(pyridin-2-ylmethyl)-carbamoyl]-1H-pyrrol-2-yl)-3,5-dihydroxy-hept-6-enoic acid;
(3R,5R)-7-[5-(3-Dimethylcarbamoyl-phenylcarbamoyl)-3-(4-fluoro-phenyl)-1-isopropyl-4-phenyl-1H-pyrrol-2-yl]-3,5-dihydroxy-heptanoic acid;
trans-(3R,5S)-7-[5-(3-Dimethylcarbamoyl-phenylcarbamoyl)-3-(4-fluoro-phenyl)-1-isopropyl-4-phenyl-1H-pyrrol-2-yl]-3,5-dihydroxy-hept-6-enoic acid;
(3R,5R)-3-{[5-(6-Carboxy-3,5-dihydroxy-hexyl)-4-(4-fluoro-phenyl)-1-isopropyl-3-phenyl-1H-pyrrole-2-carbonyl]-amino}-benzoic acid methyl ester;
(3R,5R)-3-{[5-(6-Carboxy-3,5-dihydroxy-hexyl)₄-(4-fluoro-phenyl)-1-isopropyl-3-phenyl-1H-pyrrole-2-carbonyl]-amino}-benzoic acid;
trans-(3R,5S)-3-{[5-(6-Carboxy-3,5-dihydroxy-hex-1-enyl)-4-(4-fluoro-phenyl)-1-isopropyl-3-phenyl-1H-pyrrole-2-carbonyl]-amino}-benzoic acid methyl ester;
trans-(3R,5S)-3-{[5-(6-Carboxy-3,5-dihydroxy-hex-1-enyl)-4-(4-fluoro-phenyl)-1-isopropyl-3-phenyl-1H-pyrrole-2-carbonyl]-amino}-benzoic acid;
(3R,5R)-7-[3-(4-Fluoro-phenyl)-1-isopropyl-5-(5-methyl-isoxazol-3-ylcarbamoyl)-4-phenyl-1H-pyrrol-2-yl]-3,5-dihydroxy-heptanoic acid;
trans-(3R, 5 S)-7-[3-(4-Fluoro-phenyl)-1-isopropyl-5-(5-methyl-isoxazol-3-ylcarbamoyl)-4-phenyl-1H-pyrrol-2-yl]-3,5-dihydroxy-hept-6-enoic acid;
(3R,5R)-7-[3-(4-Fluoro-phenyl)-1-isopropyl-5-(4-methyl-pyrimidin-2-ylcarbamoyl)-4-phenyl-1H-pyrrol-2-yl]-3,5-dihydroxy-heptanoic acid;
trans-(3R,5S)-7-[3-(4-Fluoro-phenyl)-1-isopropyl-5-(4-methyl-pyrimidin-2-ylcarbamoyl)-4-phenyl-1H-pyrrol-2-yl]-3,5-dihydroxy-hept-6-enoic acid;
(3R,5R)-7-[3-(4-Fluoro-phenyl)-1-isopropyl-5-(3-oxazol-2-yl-phenylcarbamoyl)-4-phenyl-1H-pyrrol-2-yl]-3,5-dihydroxy-heptanoic acid;
trans-(3R,5S)-7-[3-(4-Fluoro-phenyl)-1-isopropyl-5-(3-oxazol-2-yl-phenylcarbamoyl)-4-phenyl-1H-pyrrol-2-yl]-3,5-dihydroxy-hept-6-enoic acid;
(3R,5R)-7-[3-(4-Fluoro-phenyl)-1-isopropyl-5-(4-oxazol-2-yl-phenylcarbamoyl)-4-phenyl-1H-pyrrol-2-yl]-3,5-dihydroxy-heptanoic acid;
trans-(3R,5S)-7-[3-(4-Fluoro-phenyl)-1-isopropyl-5-(4-oxazol-2-yl-phenylcarbamoyl)-4-phenyl-1H-pyrrol-2-yl]-3,5-dihydroxy-hept-6-enoic acid;
(3R,5R)-7-[3-(4-Fluoro-phenyl)-1-isopropyl-4-phenyl-5-(pyrimidin-2-ylcarbamoyl)-1H-pyrrol-2-yl]-3,5-dihydroxy-heptanoic acid;
trans-(3R, 5 S)-7-[3-(4-Fluoro-phenyl)-1-isopropyl-4-phenyl-5-(pyrimidin-2-ylcarbamoyl)-1H-pyrrol-2-yl]-3,5-dihydroxy-hept-6-enoic acid;
and pharmaceutically acceptable salts, esters and amides thereof.

50. A compound selected from the group consisting of: trans-(3R,5S)-7-[5-(4-Dimethylcarbamoyl-phenylcarbamoyl)-3,4-bis-(4-fluoro-phenyl)-1-isopropyl-1H-pyrrol-2-yl]-3,5-dihydroxy-hept-6-enoic acid;
(3R,5R)-7-[5-(4-Dimethylcarbamoyl-phenylcarbamoyl)-3,4-bis-(4-fluoro-phenyl)-1-isopropyl-1H-pyrrol-2-yl]-3,5-dihydroxy-heptanoic acid;
(3R)-7-[5-(4-Dimethylcarbamoyl-phenylcarbamoyl)-3,4-bis-(4-fluoro-phenyl)-1-isopropyl-1H-pyrrol-2-yl]-3-hydroxy-heptanoic acid;
trans-(3R,5S)-7-[5-(4-Dimethylsulfamoyl-benzylcarbamoyl)-3,4-bis-(4-fluoro-phenyl)-1-isopropyl-1H-pyrrol-2-yl]-3,5-dihydroxy-hept-6-enoic acid;
(3R,5R)-7-[5-(4-Dimethylsulfamoyl-benzylcarbamoyl)-3,4-bis-(4-fluoro-phenyl)-1-isopropyl-1H-pyrrol-2-yl]-3,5-dihydroxy-heptanoic acid;
trans-(3R,5S)-7-{3,4-Bis-(4-fluoro-phenyl)-1-isopropyl-5-[(pyridin-2-ylmethyl)-carbamoyl]-1H-pyrrol-2-yl}-3,5-dihydroxy-hept-6-enoic acid;
(3R,5R)-7-{3,4-Bis-(4-fluoro-phenyl)-1-isopropyl-5-[(pyridin-2-ylmethyl)-carbamoyl]-1H-pyrrol-2-yl}-3,5-dihydroxy-heptanoic acid;
trans-(3R,5S)-7-[5-(3-Diemethylsulfamoyl-benzylcarbamoyl)-3,4-bis-(4-fluoro-phenyl)-1-isopropyl-1H-pyrrol-2-yl]-3,5-dihydroxy-hept-6-enoic acid;
(3R,5R)-7-[5-(3-Diemethylsulfamoyl-benzylcarbamoyl)-3,4-bis-(4-fluoro-phenyl)-1-isopropyl-1H-pyrrol-2-yl]-3,5-dihydroxy-heptanoic acid;
trans-(3R,5S)-7-[5-(3-Dimethylcarbamoyl-phenylcarbamoyl)-3,4-bis-(4-fluoro-phenyl)-1-isopropyl-1H-pyrrol-2-yl]-3,5-dihydroxy-hept-6-enoic acid;
(3R,5R)-7-[5-(3-Dimethylcarbamoyl-phenylcarbamoyl)-3,4-bis-(4-fluoro-phenyl)-1-isopropyl-1H-pyrrol-2-yl]-3,5-dihydroxy-heptanoic acid;
trans-(3R,5S)-{[5-(6-Carboxy-3,5-dihydroxy-hex-1-enyl)-3,4-bis-(4-fluoro-phenyl)-1-isopropyl-1H-pyrrole-2-carbonyl]-amino}-benzoic acid methyl ester;
(3R,5R)-{[5-(6-Carboxy-3,5-dihydroxy-hexyl)-3,4-bis-(4-fluoro-phenyl)-1-isopropyl-1H-pyrrole-2-carbonyl]-amino)}-benzoic acid methyl ester;
trans-(3R,5 S)-7-[3,4-Bis-(4-fluoro-phenyl)-1-isopropyl-5-(4-methyl-pyrimidin-2-ylcarbamoyl)-1H-pyrrol-2-yl]-3,5-dihydroxy-hept-6-enoic acid;
(3R,5R)-7-[3,4-Bis-(4-fluoro-phenyl)-1-isopropyl-5-(4-methyl-pyrimidin-2-ylcarbamoyl)-1H-pyrrol-2-yl]-3,5-dihydroxy-heptanoic acid;
trans-(3R,5S)-7-[3,4-Bis-(4-fluoro-phenyl)-1-isopropyl-5-(4-oxazol-2-yl-phenylcarbamoyl)-1H-pyrrol-2-yl]-3,5-dihydroxy-hept-6-enoic acid;
(3R,5R)-7-[3,4-Bis-(4-fluoro-phenyl)-1-isopropyl-5-(4-oxazol-2-yl-phenylcarbamoyl)-1H-pyrrol-2-yl]-3,5-dihydroxy-heptanoic acid;
trans-(3R,5S)-7-[3,4-Bis-(4-fluoro-phenyl)-1-isopropyl-5-(3-oxazol-2-yl-phenylcarbamoyl)-1H-pyrrol-2-yl]-3,5-dihydroxy-hept-6-enoic acid;
(3R,5R)-7-[3,4-Bis-(4-fluoro-phenyl)-1-isopropyl-5-(3-oxazol-2-yl-phenylcarbamoyl)-1H-pyrrol-2-yl]-3,5-dihydroxy-heptanoic acid;
trans-(3R,5 S)-7-[3,4-Bis-(4-fluoro-phenyl)-1-isopropyl-5-(5-methyl-isoxazol-3-ylcarbamoyl)-1H-pyrrole-2-yl]-3,5-dihydroxy-hept-6-enoic acid;
(3R,5R)-7-[3,4-Bis-(4-fluoro-phenyl)-1-isopropyl-5-(5-7methyl-isoxazol-3-ylcarbamoyl)-1H-pyrrole-2-yl]-3,5-dihydroxy-heptanoic acid;
and pharmaceutically acceptable salts, esters and amides thereof.

51. A compound selected from the group consisting of: (3R,5S)-7-[5-(4-Benzyloxy-phenylcarbamoyl)-3,4-bis-(4-fluoro-phenyl)-1-isopropyl-1H-pyrrol-2-yl]-3,5-dihydroxy-hept-6-enoic acid;
(3R,5S)-7-[3,4-Bis-(4-fluoro-phenyl)-1-isopropyl-5-(4-methoxycarbonyl-benzylcarbamoyl)-1H-pyrrol-2-yl]-3,5-dihydroxy-hept-6-enoic acid;
(3R,5R)-7-[3,4-Bis-(4-fluoro-phenyl)-5-(4-hydroxy-phenylcarbamoyl)-1-isopropyl-1H-pyrrol-2-yl]-3,5-dihydroxy-heptanoic acid;
(3R,5R)-7-[5-(4-Benzyloxy-phenylcarbamoyl)-3,4-bis-(4-fluoro-phenyl)-1-isopropyl-1H-pyrrol-2-yl]-3,5-dihydroxy-heptanoic acid;
(3R,5R)-7-[3,4-Bis-(4-fluoro-phenyl)-1-isopropyl-5-(4-methoxycarbonyl-benzylcarbamoyl)-1H-pyrrol-2-yl]-3,5-dihydroxy-heptanoic acid;
(3R,5 S)-7-[5-(2-Benzyloxy-phenylcarbamoyl)-3,4-bis-(4-fluoro-phenyl)-1-isopropyl-1H-pyrrol-2-yl]-3,5-dihydroxy-hept-6-enoic acid;
(3R,5R)-7-[5-(4-Carboxy-benzylcarbamoyl)-3,4-bis-(4-fluoro-phenyl)-1-isopropyl-1H-pyrrol-2-yl]-3,5-dihydroxy-heptanoic acid;
(3R,5S)-7-[5-(3-Benzyloxy-phenylcarbamoyl)-3,4-bis-(4-fluoro-phenyl)-1-isopropyl-1H-pyrrol-2-yl]-3,5 dihydroxy-hept-6-enoic acid;
(3R,5S)-7-[3,4-Bis-(4-fluoro-phenyl)-1-isopropyl-5-(3-sulfamoyl-phenylcarbamoyl)-1H-pyrrol-2-yl]-3,5-dihydroxy-hept-6-enoic acid;
(3R,5R)-7-[3,4-Bis-(4-fluoro-phenyl)-5-(3-hydroxy-phenylcarbamoyl)-1-isopropyl-1H-pyrrol-2-yl]-3,5-dihydroxy-heptanoic acid;
(3R,5R)-7-[3,4-Bis-(4-fluoro-phenyl)-5-(2-hydroxy-phenylcarbamoyl)-1-isopropyl-1H-pyrrol-2-yl]-3,5-dihydroxy-heptanoic acid;
(3R,5S)-7-[3,4-Bis-(4-fluoro-phenyl)-1-isopropyl-5-(3-methoxy-phenylcarbamoyl)-1H-pyrrol-2-yl]-3,5-dihydroxy-hept-6-enoic acid;
(3R,5R)-7-[3,4-Bis-(4-fluoro-phenyl)-1-isopropyl-5-(3-sulfamoyl-phenylcarbamoyl)-1H-pyrrol-2-yl]-3,5-dihydroxy-heptanoic acid;
(3R,5R)-7-[3,4-Bis-(4-fluoro-phenyl)-0.1-isopropyl-5-(3-methoxy-phenylcarbamoyl)-1H-pyrrol-2-yl]-3,5-dihydroxy-heptanoic acid;
(3R,5S)-7-[5-(3-Chloro-phenylcarbamoyl)-3,4-bis-(4-fluoro-phenyl)-1-isopropyl-1H-pyrrol-2-yl]-3,5-dihydroxy-hept-6-enoic acid;
(3R,5S)-7-[5-(3-Ethyl-phenylcarbamoyl)-3,4-bis-(4-fluoro-phenyl)-1-isopropyl-1H-pyrrol-2-yl]-3,5-dihydroxy-hept-6-enoic acid;
(3R,5R)-7-[5-(3-Ethyl-phenylcarbamoyl)-3,4-bis-(4-fluoro-phenyl)-1-isopropyl-1H-pyrrol-2-yl]-3,5-dihydroxy-heptanoic acid;
(3R,5R)-7-[5-(3-Chloro-phenylcarbamoyl)-3,4-bis-(4-fluoro-phenyl)-1-isopropyl-1H-pyrrol-2-yl]-3,5-dihydroxy-heptanoic acid;
and pharmaceutically acceptable salts, esters and amides thereof.

52. A compound selected from the group consisting of: (3R,5R)-7-[3,4-bis(4-fluorophenyl)-5-(4-fluoro-phenylcarbamoyl)-1-isopropyl-1H-pyrrol-2-yl]-3,5-dihydroxy-heptanoic acid;
(3R,5R)-7-[3,4-bis(4-fluoro-phenyl)-5-(3-fluorophenylcarbamoyl)-1-isopropyl-1H-pyrrol-2-yl]-3,5-dihydroxy-heptanoic acid;
(3R,5R)-7-[5-(4-Carboxymethyl-phenylcarbamoyl)-3,4-bis(4-fluorophenyl)-1-isopropyl-1H-pyrrol-2-yl]-3,5-dihydroxy-heptanoic acid;
(3R,5R)-7-[5-(4-ethylpiperazine-1-carbonyl)-3,4-bis(4-fluorophenyl)-1-isopropyl-1H-pyrrol-2-yl]-3,5-dihydroxy-heptanoic acid;
(3R,5R)-7-[5-(4-carbamoyl-phenylcarbamoyl)-3,4-bis(4-fluorophenyl)-1-isopropyl-1H-pyrrol-2-yl]-3,5-dihydroxy-heptanoic acid;
(3R,5R)-7-[3,4-bis(4-fluorophenyl)-1-isopropyl-5-(4-methoxycarbonyl-phenylcarbamoyl)-1H-pyrrol-2-yl]-3,5-dihydroxy-heptanoic acid;
(3R,5R)-7-[3,4-bis-(4-fluorophenyl)-1-isopropyl-5-(4-sulfamoyl-phenylcarbamoyl)-1H-pyrrol-2-yl]-3,5-dihydroxy-heptanoic acid;
(3R,5R)-7-[5-(3,5-difluorophenylcarbamoyl)-3-(4-fluorophenyl)-1-isopropyl-4-phenyl-1H-pyrrol-2-yl]-3,5-dihydroxy-heptanoic acid;
(3R,5R)-7-[3-(4-fluoro-phenyl)-1-isopropyl-4-phenyl-5-(pyridin-2-ylcarbamoyl)-1H-pyrrol-2-yl]-3,5-dihydroxy-heptanoic acid; and
pharmaceutically acceptable salts, esters and amides thereof.

53. A compound selected from the group consisting of:
(4R,6R)-(6-[2-[3,4-Bis-(4-fluoro-phenyl)-1-isopropyl-1H-pyrrol-2-yl]-ethyl)-2,2-dimethyl-[1,3]dioxan-4-yl)-acetic acid;
6-{[5-(6-Carboxy-3,5-dihydroxy-hexyl)-4-(4-fluoro-phenyl)-1-isopropyl-3-phenyl-1H-pyrrole-2-carbonyl]-amino}-nicotinic acid;
7-[5-(Acetylamino-methyl)-3-(4-fluoro-phenyl)-1-isopropyl-4-phenyl-1H-pyrrol-2-yl]-3,5-dihydroxy-heptanoic acid; and pharmaceutically acceptable salts, esters and amides thereof.

54. A pharmaceutical composition comprising: a therapeutically effective amount of a first compound, said first compound having a Formula I, 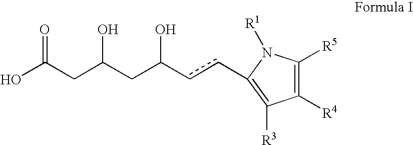
or a pharmaceutically acceptable salt, ester, amide, stereoisomer or prodrug thereof, or a pharmaceutically acceptable salt of the prodrug,
wherein R¹ is lower alkyl, optionally substituted with a halogen;
R³ is benzyl; naphthyl; C₃-C₈ cycloalkyl or C₅-C₈ cycloalkenyl, optionally one or more heteroatom(s); phenyl or phenyl substituted with one or more groups selected from fluorine, chlorine, bromine, hydroxyl or alkyl of from one to seven carbon atoms; pyridinyl or pyridinyl substituted with fluorine, chlorine, bromine, hydroxyl or alkyl of from one to seven carbon atoms;
R⁴ is H; aryl, aralkyl, heteroaryl or heteroaralkyl; optionally substituted with one or more groups selected from fluorine, chlorine, bromine, hydroxyl, (CH₂)ₙOR′, (CH₂)ₙCOOR′, (CH₂)ₙCONR′R″, (CH₂)ₙS(O)₂NR′R″, (CH₂)ₙS(O)₂R⁸, alkyl or alkoxy of from one to seven carbon atoms; C₁-C₈ alkyl or C₃-C₈ cycloalkyl; optionally substituted; aralkenyl; carbamoyl or substituted carbamoyl; carboxyl or substituted carboxyl;
R⁵ is H, I, phenyl or substituted phenyl, COOR′, R⁶R⁷NC(O)—;
—(CH₂)ₙNR⁶R⁷ or SO₂NR⁶R⁷;
R⁶ and R⁷ are each independently H; aryl, aralkyl, heteroaryl or heteroaralkyl; optionally substituted with halo, alkyl of from one to seven carbon atoms, (CH₂)ₙOR′, (CH₂)ₙCOOR′, (CH₂).CONR′R″,
(CH₂)ₙS(O)₂NR′R″, (CH₂)ₙS(O)₂R⁸, or heteroaryl;
C₁-C₁₀ alkyl, C₃-C₈ cycloalkyl or C₅-C₈ cycloalkenyl, said alkyl, cycloalkyl or cycloalkenyl optionally containing one or more heteroatoms(s); unsubstituted or substituted with OH, CO₂R′ or CONR′R″;
COOR′; C(O)R′; SO₂NHR⁸ or SO₂R⁸;
or N, R⁶ and R⁷ taken together form a 4-7 member ring, optionally containing up to 2 heteroatoms selected from O, N and S, said heteroatom(s) being optionally substituted; said ring optionally substituted with lower alkyl, OH, benzyl, phenyl, CO₂R′ or CONR′R″;
R⁸ is aryl, aralkyl, alkyl, heteroaryl or heteroaralkyl; optionally substituted;
R′ and R″ are each independently H, C₁-C₁₂ alkyl, aryl, or aralkyl, or taken together form a 4-7 member ring;
n is 0-2; and
wherein 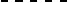
is a bond or is absent; and
a second compound, said second compound being a CETP inhibitor; a PPAR-activator, an MTP/Apo B secretion inhibitor, a cholesterol absorption inhibitor, a cholesterol synthesis inhibitor, a fibrate, niacin, an ion-exchange resin, an antioxidant, an ACAT inhibitor, or bile sequestrant; an anti-hypertensive agent; an acetylcholine esterase inhibitor; and a pharmaceutical carrier.
